(19) United States
(12) Patent Application Publication
(10) Pub. No.: US 2026/0042731 A1
(43) Pub. Date: Feb. 12, 2026

(54) (2,4-DICHLOROPHENOXY)ACETIC ACID ANALOGS

(71) Applicant: CORTEVA AGRISCIENCE LLC, INDIANAPOLIS, IN (US)

(72) Inventors: JEREMY KISTER, CARMEL, IN (US); MEI LI, WESTFIELD, IN (US); DAVID MICHAEL SIMPSON, ZIONSVILLE, IN (US); BIN WEI, CARMEL, IN (US)

(73) Assignee: CORTEVA AGRISCIENCE LLC, INDIANAPOLIS, IN (US)

(21) Appl. No.: 19/100,428

(22) PCT Filed: Aug. 3, 2023
(86) PCT No.: PCT/US2023/071586

§ 371 Date: Jan. 31, 2025

Related U.S. Application Data
(60) Provisional application No. 63/370,350, filed on Aug. 3, 2022.

(51) Int. Cl. C07C 251/64; C07C 233/91; C07C 255/61; C07C 323/58; C07D 209/48; C07D 213/78; C07D 295/28; C07D 307/68; C07D 333/22

(52) U.S. Cl. CPC C07C 251/64; C07C 233/91; C07C 255/61; C07C 323/58; C07D 209/48; C07D 213/78; C07D 295/28; C07D 307/68; C07D 333/22

(57) ABSTRACT

This disclosure includes pesticidal compounds, compositions, and related agricultural methods.

### FIELD

Aspects herein include pesticidal compositions and agricultural methods, including herbicidal compositions that are useful in controlling unwanted plants. The herbicides disclosed herein may be used alone, in various formulations, or together with other agriculturally actives including other herbicides to control weeds in various crops.

This disclosure includes (2,4-Dichlorophenoxy)acetic acid analogs. More specifically, this disclosure relates to (2,4-Dichlorophenoxy)acetic acid analogs, such as esters, amides, aminooxies, linkers, and carbon groups.

### BACKGROUND

Protecting crops from undesirable vegetation which adversely affect crop growth is a constantly recurring problem in agriculture. To help combat this problem, researchers in the field of synthetic chemistry have produced a variety of chemicals and chemical formulations effective in the control of such unwanted plant growth. Chemical herbicides of many types have been disclosed in the literature and a large number are in commercial use. However, there remains a need for compositions and methods that are effective in controlling undesirable vegetation, and which demonstrate other physiochemical characteristics that make them easy to formulate and to apply under varying field conditions.

(2,4-Dichlorophenoxy)acetic acid, also known as 2,4-D, is a known herbicide. Often 2,4-D is used as an herbicide and defoliant. Often 2,4-D is used as a weed killer in cereal crops, pastures, and orchards.

However, various forms of 2,4-D can often suffer from adverse properties, such as volatility, spray drift, vapor drift, pungent odors, limited stability, limited tank-mixability with other pesticidal actives, and/or limited formulation flexibility.

Efforts to solve the volatility problem, including preparation of water-soluble salts such as the dimethylamine salt of 2,4-D, have not been totally satisfactory because, upon volatilization of the amine, the herbicide reverts back to its initial acid form, which, in itself under certain unfavorable conditions, has sufficient volatility to cause damage to sensitive crops.

Conventional 2,4-D ester or 2,4-D dimethylamine formulations applied during the warm summer months can lead to vapor drift from the evaporation of the herbicide from sprayed surfaces and subsequent damage to highly susceptible crops such as tomatoes, cotton, soybeans, sunflowers and grapes. This may occur within hours after the herbicide application.

Furthermore, conventional 2,4-D ester or conventional 2,4-D dimethylamine formulations can suffer from formulation limitations, such as their ability to be mixed, including tank-mixed, with other actives, such as glyphosate.

A need therefore exists for alternate forms of 2,4-D that have reduced volatility, reduced spray drift, reduced volatility drift, reduced odor, improved tank-mixability with other pesticidal actives, or improved formulation flexibility.

### SUMMARY

Disclosed herein are various compounds according to various details or aspects. Such compounds may include compounds according to Formula I:

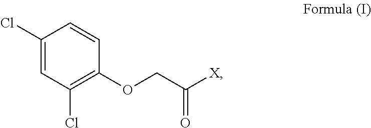

wherein X is:

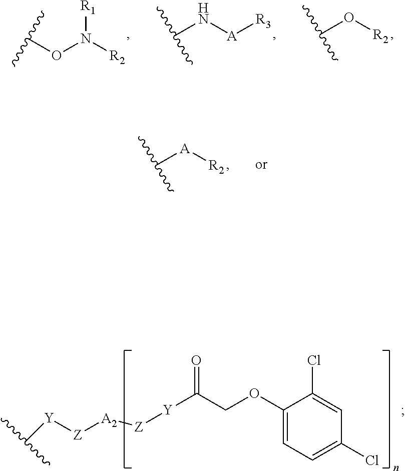

wherein
R₁ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, or heteroarylthiocarbonyl;
R₂ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl;
R₃ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl;
R₁ and R₂ together form a substituted double bond, a 3-membered ring, a 4-membered ring, a 5-membered ring, a 6-membered ring, or a 7-membered ring;
A is independently O, Si, NR, and S(O)ₘ;
   A₂ is independently O, N, S(O)ₘ, substituted alkyl, heteroalkyl, alkenyl, alkynyl, aryl, heteroaryl, alkylaryl, alkylheteroaryl;
Y is independently O, N, S;
Z is independently O, N, S(O)ₘ, substituted alkyl, alkenyl, alkynyl, aryl, heteroaryl, alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl, —N═R₄R₅,
R₄ and R₅ are independently substituted amino, alkoxy, alkyl, heteroalkyl, aryl, heteroaryl;
n is 1, 2, or 3; and
m is 0, 1, or 2; wherein
the compound exhibits pesticidal activity with the proviso that the compound is not 2,4-D isooctyl ester.

For example, various compounds may include the aforementioned compounds wherein X is:

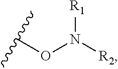

where
R₁ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, or heteroarylthiocarbonyl; and
R₂ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl.

As yet another example, various compounds may include compounds according to Formula I wherein X is:

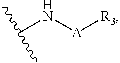

where
R₃ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl;
A is independently O, Si, NR, and S(O)ₘ, substituted alkyl, alkenyl, alkynyl, aryl, heteroaryl, alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl, —N═R₄R₅,
R₄ and R₅ are independently substituted amino, alkoxy, alkyl, heteroalkyl, aryl, heteroaryl; and
m is 0, 1, or 2.

Further examples include various compounds may include compounds according to Formula I wherein X is:

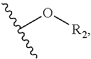

where
R₂ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl.

Also included are compounds according to Formula I wherein X is:

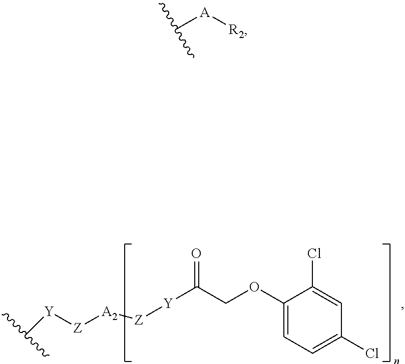

where A, R₂, Y, Z, and A₂ are as previously described.

Further examples include compounds of the following formulas:

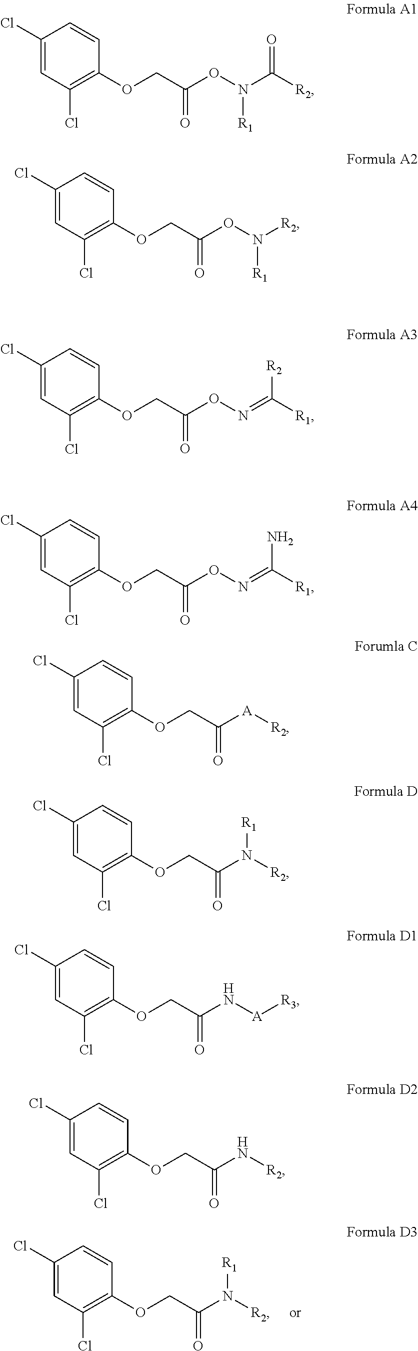

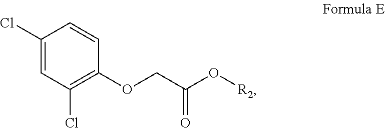

where
R₁ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, or heteroarylthiocarbonyl;
R₂ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl;
R₃ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl;
R₁ and R₂ together form a substituted double bond, a 3-membered ring, a 5-membered ring, or a 7-membered ring;
A is independently O, Si, NR, and S(O)ₘ;
   A₂ is independently O, N, S(O)ₘ, substituted alkyl, heteroalkyl, alkenyl, alkynyl, aryl, heteroaryl, alkylaryl, alkylheteroaryl;
Y is independently O, N, S;
Z is independently O, N, S(O)ₘ, substituted alkyl, alkenyl, alkynyl, aryl, heteroaryl, alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl, —N═R₄R₅,
R₄ and R₅ are independently substituted amino, alkoxy, alkyl, heteroalkyl, aryl, heteroaryl;
n is 1, 2, or 3; and
m is 0, 1, or 2

Various compositions may include any of the aforementioned compounds, where the concentration of active ingredients in the composition is from 0.001 to 98 percent by weight.

Such previously described compounds may exhibit a variety of pesticidal activity, such as herbicidal activity.

Also included herein are methods of controlling undesirable vegetation which comprises contacting the vegetation or the locus thereof with a herbicidally effective amount of the previously described compounds, or applying to the soil to prevent the emergence of vegetation a herbicidally effective amount of the previously described compounds.

Methods of controlling undesirable vegetation include methods where disclosed compounds are applied at a rate of between about 200 grams to about 2,000 grams of acid equivalent per hectare (g ae/ha), such as between about 800 g ae/ha to about 1,200 g ae/ha.

Also included are methods of controlling undesirable vegetation in a crop that has been made tolerant or resistant to 2,4-dichlorophenoxyacetic acid, which comprises contacting the vegetation or the locus thereof with or applying to the soil to prevent the emergence of vegetation, a herbicidally effective amount of a compound according to any one of the compounds disclosed herein.

### DETAILED DESCRIPTION

The aspect disclosed below is not intended to be exhaustive or limit the disclosure to the precise form disclosed in the following detailed description. Rather, the aspects are chosen and described so that others skilled in the art may utilize its teachings.

While various concepts of the present disclosure are illustrated and described in detail in the figures and the description herein, results in the figures and their description are to be considered as exemplary and not restrictive in character; it being understood that only the illustrative aspects are shown and described and that all changes and modifications that come within the spirit of the disclosure are desired to be protected.

### Definitions

Unless defined otherwise, the scientific and technology nomenclatures have the same meaning as commonly understood by a person of ordinary skill in the art pertaining to this disclosure.

As used herein, unless explicitly stated otherwise or clearly implied otherwise, the term “about” refers to a range of values plus or minus 10 percent, e.g., about 1.0 encompasses values from 0.9 to 1.1.

As used herein, unless explicitly stated otherwise or clearly implied otherwise, the term “agriculturally active” compound includes any compound that may be of benefit to agriculture, including compounds that either alone or in combination with other compounds inhibit the growth of plants deemed undesirable in a given agricultural setting.

The examples given in the definitions are generally non-exhaustive and must not be construed as limiting the invention disclosed in this document. It is understood that a substituent should comply with chemical bonding rules and steric compatibility constraints in relation to the molecule to which it is attached.

As used herein, the term “alkanes” may be understood to include straight chain, branched, or cyclic, substituted, or unsubstituted hydrocarbons, which comprise one or more carbon-carbon bonds. Elements and groups that can be used to substitute alkanes include, but are not limited to, groups which include oxygen, nitrogen, sulfur, halogen, or metals, the linkage between the hydrocarbon and the substituent may be via a carbon-carbon bond or through an element other than carbon including, but not limited to, oxygen, nitrogen, and sulfur.

As used herein, the term “alkenes” may be understood to refer to straight chain, branched, or cyclic substituted or unsubstituted hydrocarbons, which include at least one carbon-carbon double bond. Elements and groups that can be used to substitute alkenes include, but are not limited to, groups which include oxygen, nitrogen, sulfur, halogen, or metals, the linkage between the hydrocarbon and the substituent may be via a carbon-carbon bond or through an element other than carbon including, but not limited to, oxygen, nitrogen, and sulfur.

As used herein, the term “alkynes” may be understood to include reference to straight chain or branched or cyclic, substituted, or unsubstituted hydrocarbon, which include at least one carbon-carbon triple bond. Elements and groups that can be used to substitute alkynes include, but are not limited to, groups which include oxygen, nitrogen, sulfur, halogen, or metals, the linkage between the hydrocarbon and the substituent may be via a carbon-carbon bond or through an element other than carbon including, but not limited to, oxygen, nitrogen, and sulfur.

As used herein, the term “halo” may be understood to refer to one or more of the following elements: fluoro, chloro, bromo, and iodo.

As used herein, the term “aryl” may be understood to refer to any group comprising at least one aromatic ring. The aromatic ring itself may be substituted or unsubstituted. Elements and groups that can be used to substitute aryls include, but are not limited to, groups which include oxygen, nitrogen, sulfur, halogen, or metals, the linkage between the hydrocarbon and the substituent may be via a carbon-carbon bond or through an element other than carbon including, but not limited to, oxygen, nitrogen, and sulfur.

As used herein, the term “heteroaryl” may be understood to include two aromatic rings which include in the cyclic structure elements other than carbon, such other elements include, but are not limited to, oxygen, nitrogen, sulfur, and the like. Heteroaryl may include more than one ring structure. Heteroaryls may be substituted or unsubstituted, substituents include, but are not limited to, elements and/or groups which include oxygen, nitrogen, sulfur, halogen, or metals, the linkage between the hydrocarbon and the substituent may be via a carbon-carbon bond or through an element other than carbon including, but not limited to, oxygen, nitrogen, and sulfur.

Exemplary heteroaryls include, but are not limited to, benzofuranyl, benzoisothiazolyl, benzoisoxazolyl, benzoxazolyl, benzothienyl, benzothiazolyl, cinnolinyl, furanyl, imidazolyl, indazolyl, indolyl, isoindolyl, isoquinolinyl, isothiazolyl, isoxazolyl, oxadiazolyl, oxazolinyl, oxazolyl, phthalazinyl, pyrazinyl, pyrazolinyl, pyrazolylpyridazinyl, pyridyl, pyrimidinyl, pyrrolyl, quinazolinyl, quinolinyl, quinoxalinyl, tetrazolyl, thiazolinyl, thiazolyl, thienyl, triazinyl, and triazolyl.

Saturated heterocycles are ring structures which include one or more elements other than carbon, these elements include, but are not limited to, oxygen, nitrogen, sulfur, and the like. Exemplary saturated heterocycles include, but are not limited to, piperazinyl, piperidinyl, morpholinyl, pyrrolidinyl, oxetanyl, tetrahydrofuranyl, tetrahydrothienyl and tetrahydropyranyl.

Examples of partially unsaturated hetero cycles include, but are not limited to, 1,2,3,4-tetrahydro quinolinyl, 4.5-dihydro-oxazolyl. 4,5-dihydro-1H-pyrazolyl, 4,5-dihydro-isoxazolyl, and 2,3-dihydro-1,3,4-oxadiazolyl.

As used herein, unless explicitly stated otherwise or clearly implied otherwise, the term “alkyl” refers to any of the following: alkanes, alkenes, or alkynes, which are straight chained, branched, or cyclic, substituted, or unsubstituted; substituents may include, but are not limited to, halogens, aminos, iminos, sulfones, sulfoxides, aryls, heteroaryl, esters, ketones, aldehydes, alcohols, acids, and/or esters. Unless explicitly stated otherwise, any compound capable of existing as an isomer, a stereoisomer, or a tautomer may be used in any of its herbicidally active isoforms, configurations, or tautomers.

As used herein, unless explicitly stated otherwise or clearly implied otherwise, the term “AMATA” refers to Amaranthus rudis (common waterhemp)

As used herein, unless explicitly stated otherwise or clearly implied otherwise, the term “AMBEL” refers to Ambrosia artemisiifolia (common ragweed).

As used herein, unless explicitly stated otherwise or clearly implied otherwise, the term “ABUTH” refers to Abutilon theophrasti (velvetleaf).

As used herein, unless explicitly stated otherwise or clearly implied otherwise, the term “ERICA” refers to glyphosate resistant Conyza canadensis (L.) (horseweed).

As used herein, unless explicitly stated otherwise or clearly implied otherwise, the term “GLXMA” refers to Glycine max (L.) Merr., (soybean).

As used herein, unless explicitly stated otherwise or clearly implied otherwise, the term “DCM” refers to dichloromethane.

As used herein, unless explicitly stated otherwise or clearly implied otherwise, the term “EtOAC” refers to ethyl acetate.

As used herein, unless explicitly stated otherwise or clearly implied otherwise, the term “DMF” refers to dimethyl formamide.

As used herein, unless explicitly stated otherwise or clearly implied otherwise, the term “K₂CO₃” refers to potassium carbonate.

As used herein, unless explicitly stated otherwise or clearly implied otherwise, the term “EDC” refers to 1-ethyl-3-(3-dimethylaminopropyl) carbodiimide.

As used herein, unless explicitly stated otherwise or clearly implied otherwise, the term “DMAP” refers to 4-dimethylaminopyridine.

As used herein, unless explicitly stated otherwise or clearly implied otherwise, the term “DIPEA” refers to N,N-diisopropylethylamine.

As used herein, unless explicitly stated otherwise or clearly implied otherwise, the term “Et₃N” refers to triethylamine.

As used herein, unless explicitly stated otherwise or clearly implied otherwise, the term “HPLC” refers to high-performance liquid chromatography.

As used herein, unless explicitly stated otherwise or clearly implied otherwise, the term “LC-MS” refers to liquid chromatography coupled with mass spectrometry.

As used herein, the modifier “about” used in connection with a quantity is inclusive of the stated value and has the meaning dictated by the context (for example, it includes at least the degree of error associated with the measurement of the particular quantity). When used in the context of a range, the modifier “about” should also be considered as disclosing the range defined by the absolute values of the two endpoints. For example, the range “from about 2 to about 4” also discloses the range “from 2 to 4.”

As previously stated, this disclosure includes (2,4-Dichlorophenoxy)acetic acid analogs. More specifically, this disclosure relates to (2,4-Dichlorophenoxy)acetic acid analogs (“2,4-D analogs”), such as esters, amides, aminooxies, linkers, and carbon groups.

Without being limited to any theory, it is believed that various 2,4-D analogs can have improved properties prior to or during application. Then, in the soil or in-planta, the 2,4-D analog can be broken into the active form of 2,4-D acid. Thus, various 2,4-D analogs disclosed herein can benefit from the aforementioned improved properties and yet have an acceptable pesticidal profile.

Disclosed herein are 2,4-D analogs that can be converted into 2,4-D during or after application. For example, disclosed herein are pesticidal compositions of the following Formula (I):

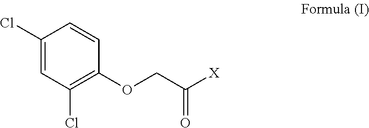

where X is any group that can be converted to a carboxylic acid during or after application, such as in the soil or in-planta. For example, some of the aspects, X may be aminooxy, amides, carbon chains, esters, or linker groups.

For example, in some aspects, X may be:

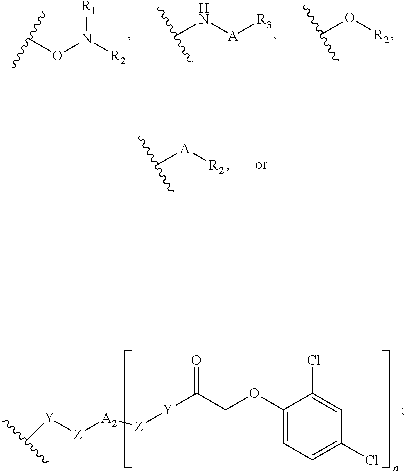

where
R₁, R₂, R₃, Y, Z, A, A₂, and n are as described below.

For example, in some aspects, when X is an aminooxy group, the aminooxy groups may include some of the following exemplary Formulas:

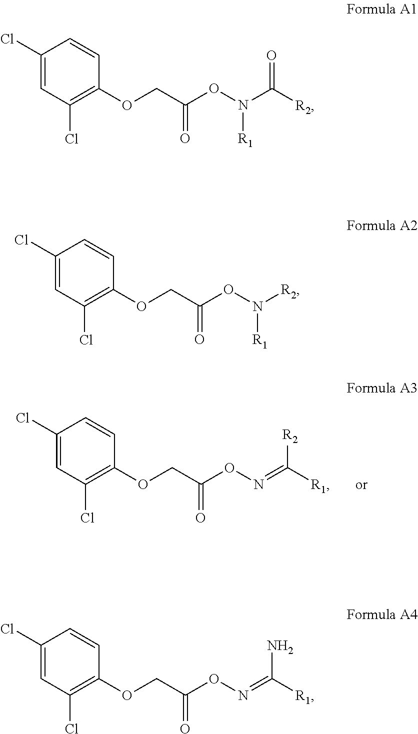

wherein
R₁ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, or heteroarylthiocarbonyl;
R₂ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl; and
R₁ and R₂ together form a substituted double bond, a 3-membered ring, a 5-membered ring, or a 7-membered ring.

For example, in some aspects, when X is a linker group, the linker groups may connect two or more 2,4-D molecules in one analog, for example as the following Formula B:

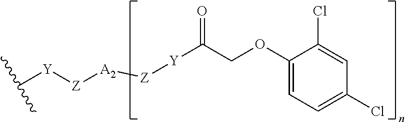

where A₂ is independently O, N, S(O)ₘ, substituted alkyl, heteroalkyl, alkenyl, alkynyl, aryl, heteroaryl, alkylaryl, alkylheteroaryl;
Y is independently O, N, S;
Z is independently O, N, S(O)ₘ with m=0 or 1 or 2, substituted alkyl, alkenyl, alkynyl, aryl, heteroaryl, alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl, —N═R₄R₅,
R₄ and R₅ are independently substituted amino, alkoxy, alkyl, heteroalkyl, aryl, heteroaryl;
n is 1, 2, or 3; and
m is 0, 1, or 2.

Exemplary linker groups may include some of the following exemplary Formula B:

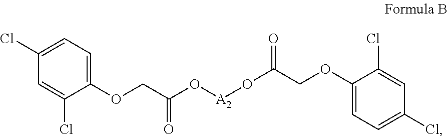

wherein A₂ is as previously described.

In some aspects, when X is a carbon group, the carbon groups may include some of the following exemplary Formula C:

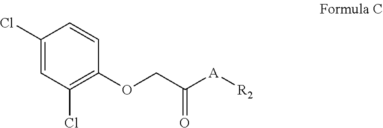

where R₂ is as previously described.

In various aspects when X is an amide group, the amide groups may include compounds within the following exemplary Formula D:

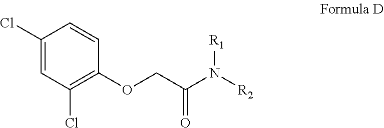

where R₁ and R₂ are as previously described.

Some exemplary compounds within the disclosure of Formula D, include the following exemplary compounds:

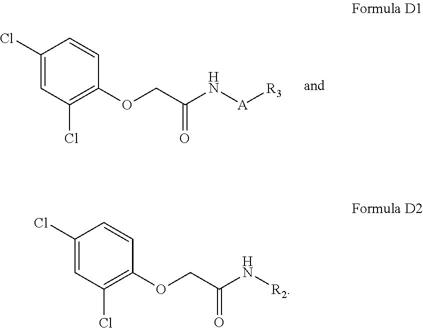

where A, R₁, R₂, and R₃ are as previously described.

In some aspects, when X forms an ester, the ester groups may include some of the following exemplary Formula E:

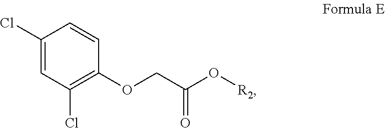

where R₂ is as previously described.

As used herein, herbicide means a compound, e.g., an active ingredient, that kills, controls, or otherwise adversely affects the survival, growth and/or reproduction of plants.

As used herein, a herbicidally effective or vegetation controlling amount is an amount of active ingredient which causes an adversely modifying effect to the vegetation e.g., causing deviations from natural development, killing, effecting regulation, causing desiccation, causing retardation, and the like.

As used herein, controlling undesirable vegetation means preventing, reducing, killing, or otherwise adversely modifying the development of plants and vegetation. Described herein are methods of controlling undesirable vegetation through the application of certain herbicide combinations or compositions. Methods of application include, but are not limited to, applications to the vegetation or locus thereof, e.g., application to the area adjacent to the vegetation, as well as preemergence, postemergence, foliar (broadcast, directed, banded, spot, mechanical, over-the-top, or rescue), and in-water applications (emerged and submerged vegetation, broadcast, spot, mechanical, water-injected, granular broadcast, granular spot, shaker bottle, or stream spray) via hand, backpack, machine, tractor, or aerial (airplane and helicopter) application methods.

As used herein, plants and vegetation include, but are not limited to, germinant seeds, emerging seedlings, plants emerging from vegetative propagules, immature vegetation, and established vegetation.

As used herein, agriculturally acceptable salts and esters refer to salts and esters that exhibit herbicidal activity, or that are or can be converted in plants, water, or soil to the referenced herbicide. Exemplary agriculturally acceptable esters are those that are or can be hydrolyzed, oxidized, metabolized, or otherwise converted, e.g., in plants, water, or soil, to the corresponding carboxylic acid which, depending on the pH, may be in the dissociated or undissociated form.

Exemplary salts include those derived from alkali or alkaline earth metals and those derived from ammonia and amines. Exemplary cations include sodium, potassium, magnesium, and ammonium cations of the formula:

Herbicidal activity is exhibited by the compounds when they are applied directly to the plant or to the locus of the plant at any stage of growth. The effect observed depends upon the plant species to be controlled, the stage of growth of the plant, the application parameters of dilution and spray drop size, the particle size of solid components, the environmental conditions at the time of use, the specific compound employed, the specific adjuvants and carriers employed, the soil type, and the like, as well as the amount of chemical applied. These and other factors can be adjusted to promote nonselective or selective herbicidal action. In some aspects, the compositions described herein are applied as a post-emergence application, pre-emergence application, or in-water application to flooded paddy rice or water bodies (e.g., ponds, lakes, and streams), to relatively immature undesirable vegetation to achieve the maximum control of weeds.

In some aspects, the compositions and methods provided herein are utilized to control weeds in crops, including but not limited to direct-seeded, water-seeded and transplanted rice, cereals, wheat, barley, oats, rye, sorghum, corn/maize, sugarcane, sunflower, oilseed rape, canola, sugar beet, soybean, cotton, pineapple, pastures, grasslands, rangelands, fallow-land, turf, tree and vine orchards, aquatics, plantation crops, vegetables, industrial vegetation management (IVM) and rights of way (ROW).

With respect to the methods, in certain aspects, the methods comprise contacting the undesirable vegetation or locus thereof or applying to the soil or water to prevent the emergence or growth of vegetation or a composition described herein. In some aspects, the composition is applied at an application rate sufficient to control, inhibit, or promote the growth of at least one plant. In some aspects the compound is applied, based on the total amount of active ingredients in the composition, in at least one of the following ranges: of from about 200 grams to about 2,000 grams of acid equivalent per hectare (g ae/ha); in some aspects the composition is applied at an application rate of between about 400 grams to about 1,500 grams g ae/ha; in some aspects composition is applied at an application rate of between about 560 grams to about 1,200 grams g ae/ha; composition is applied at an application rate of between about 800 gram to about 1,120 grams g ae/h. In some aspects, the methods comprise contacting the undesirable vegetation or locus thereof or applying to the soil or water to prevent the emergence or growth of vegetation.

The compositions and methods provided herein are utilized to control undesirable vegetation. Undesirable vegetation includes, but is not limited to, undesirable vegetation that occurs in rice, cereals, wheat, barley, oats, rye, pastures, grasslands, rangelands, fallow-land, row crops (e.g., corn/maize, sugarcane, sunflower, oilseed rape, canola, sugar beet, soybean, cotton), turf, trees and vine orchards, plantation crops, vegetables, ornamental species, aquatic or non-crop settings (e.g., rights-of-way, industrial vegetation management).

In some aspects, the methods provided herein are utilized to control undesirable vegetation in rice. In certain aspects, the undesirable vegetation is Brachiaria platyphylla (Groseb.) Nash or Urochloa platyphylla (Nash) R. D. Webster (broadleaf signalgrass, BRAPP), Digitaria sanguinalis (L.) Scop. (large crabgrass, DIGSA), Echinochloa species (ECHSS), Echinochloa crus-galli (L.) P. Beauv. (barnyardgrass, ECHCG), Echinochloa crus-pavonis (Kunth) Schult. (gulf cockspur, ECHCV), Echinochloa colonum (L.) LINK (junglerice, ECHCO), Echinochloa oryzoides (Ard.) Fritsch (early watergrass, ECHOR), Echinochloa oryzicola (Vasinger) Vasinger (late watergrass, ECHPH), Echinochloa phyllopogon (Stapf) Koso-Pol. (rice barnyardgrass, ECHPH), Echinochloa polystachya (Kunth) Hitchc. (creeping river grass, ECHPO), Ischaemum rugosum Salisb. (saramollagrass, ISCRU), Leptochloa chinensis (L.) Nees (Chinese sprangletop, LEFCH), Leptochloa fascicularis (Lam.) Gray (bearded sprangletop, LEFFA), Leptochloa panicoides (Presl.) Hitchc. (Amazon sprangletop, LEFPA), Oryza species (red and weedy rice, ORYSS), Panicum dichotomiflorum (L.) Michx. (fall panicum, PANDI), Paspalum dilatatum Poir. (dallisgrass, PASDI), Rottboellia cochinchinensis (Lour.) W. D. Clayton (itchgrass, ROOEX), Cyperus species (CYPSS), Cyperus difformis L. (smallflower flatsedge, CYPDI), Cyperus dubius Rottb. (MAPDU), Cyperus esculentus L. (yellow nutsedge, CYPES), Cyperus iria L. (rice flatsedge, CYPIR), Cyperus rotundus L. (purple nutsedge, CYPRO), Cyperus serotinus Rottb./C. B. Clarke (tidalmarsh flatsedge, CYPSE), Eleocharis species (ELOSS), Fimbristylis miliacea (L.) Vahl (globe fringerush, FIMMI), Schoenoplectus species (SCPSS), Schoenoplectus juncoides Roxb. (Japanese bulrush, SCPJU), Bolboschoenus maritimus (L.) Palla or Schoenoplectus maritimus L. Lye (sea clubrush, SCPMA), Schoenoplectus mucronatus L. (ricefield bulrush, SCPMU), Aeschynomene species, (jointvetch, AESSS), Alternanthera philoxeroides (Mart.) Griseb. (alligatorweed, ALRPH), Alisma plantago-aquatica L. (common waterplantain, ALSPA), Amaranthus species, (pigweeds and amaranths, AMASS), Ammannia coccinea Rottb. (redstem, AMMCO), Commelina benghalensis L. (Benghal dayflower, COMBE), Eclipta alba (L.) Hassk. (American false daisy, ECLAL), Heteranthera limosa (SW.) Willd./Vahl (ducksalad, HETLI), Heteranthera reniformis R. & P. (roundleaf mudplantain, HETRE), Ipomoea species (morningglories, IPOSS), Ipomoea hederacea (L.) Jacq. (ivy leaf morningglory, IPOHE), Lindernia dubia (L.) Pennell (low false pimpernel, LIDDU), Ludwigia species (LUDSS), Ludwigia linifolia Poir. (southeastern primrose-willow, LUDLI), Ludwigia octovalvis (Jacq.) Raven (longfruited primrose-willow, LUDOC), Monochoria korsakowii Regel & Maack (monochoria, MOOKA), Monochoria vaginalis (Burm. F.) C. Presl ex Kuhth, (monochoria, MOOVA), Murdannia nudiflora (L.) Brenan (doveweed, MUDNU), Polygonum pensylvanicum L., (Pennsylvania smartweed, POLPY), Polygonum persicaria L. (ladysthumb, POLPE), Polygonum hydropiperoides Michx. (POLHP, mild smartweed), Rotala indica (Willd.) Koehne (Indian toothcup, ROTIN), Sagittaria species, (arrowhead, SAGSS), Sesbania exaltata (Raf.) Cory/Rydb. Ex Hill (hemp sesbania, SEBEX), or Sphenoclea zeylanica Gaertn. (gooseweed, SPDZE).

In some aspects, the methods provided herein are utilized to control undesirable vegetation in cereals. In certain aspects, the undesirable vegetation is Alopecurus myosuroides Huds. (blackgrass, ALOMY), Apera spica-venti (L.) Beauv. (windgrass, APESV), Avena fatua L. (wild oat, AVEFA), Bromus tectorum L. (downy brome, BROTE), Lolium multiflorum Lam. (Italian ryegrass, LOLMU), Phalaris minor Retz. (littleseed canarygrass, PHAMI), Poa annua L. (annual bluegrass, POANN), Setaria pumila (Poir.) Roemer & J. A. Schultes (yellow foxtail, SETLU), Setaria viridis (L.) Beauv. (green foxtail, SETVI), Amaranthus retroflexus L. (redroot pigweed, AMARE), Brassica species (BRSSS), Chenopodium album L. (common lambsquarters, CHEAL), Cirsium arvense (L.) Scop. (Canada thistle, CIRAR), Galium aparine L. (catchweed bedstraw, GALAP), Kochia scoparia (L.) Schrad. (kochia, KCHSC), Lamium purpureum L. (purple deadnettle, LAMPU), Matricaria recutita L. (wild chamomile, MATCH), Matricaria matricarioides (Less.) Porter (pineappleweed, MATMT), Papaver rhoeas L. (common poppy, PAPRH), Polygonum convolvulus L. (wild buckwheat, POLCO), Salsola tragus L. (Russian thistle, SASKR), Sinapis species (SINSS), Sinapis arvensis L. (wild mustard, SINAR), Stellaria media (L.) Vill. (common chickweed, STEME), Veronica persica Poir. (Persian speedwell, VERPE), Viola arvensis Murr. (field violet, VIOAR), or Viola tricolor L. (wild violet, VIOTR).

In some aspects, the methods provided herein are utilized to control undesirable vegetation in range and pasture, fallow land, IVM, and ROW. In certain aspects, the undesirable vegetation is Ambrosia artemisiifolia L. (common ragweed, AMBEL), Cassia obtusifolia (sickle pod, CASOB), Centaurea maculosa auct. non Lam. (spotted knapweed, CENMA), Cirsium arvense (L.) Scop. (Canada thistle, CIRAR), Convolvulus arvensis L. (field bindweed, CONAR), Daucus carota L. (wild carrot, DAUCA), Euphorbia esula L. (leafy spurge, EPHES), Lactuca serriola L./Torn. (prickly lettuce, LACSE), Plantago lanceolata L. (buckhorn plantain, PLALA), Rumex obtusifolius L. (broadleaf dock, RUMOB), Sida spinosa L. (prickly sida, SIDSP), Sinapis arvensis L. (wild mustard, SINAR), Sonchus arvensis L. (perennial sowthistle, SONAR), Solidago species (goldenrod, SOOSS), Taraxacum officinale G. H. Weber ex Wiggers (dandelion, TAROF), Trifolium repens L. (white clover, TRFRE), or Urtica dioica L. (common nettle, URTDI).

In some aspects, the methods provided herein are utilized to control undesirable vegetation found in row crops, tree and vine crops, and perennial crops. In certain aspects, the undesirable vegetation is Alopecurus myosuroides Huds. (blackgrass, ALOMY), Avena fatua L. (wild oat, AVEFA), Brachiaria decumbens Stapf. or Urochloa decumbens (Stapf) R. D. Webster (Surinam grass, BRADC), Brachiaria brizantha (Hochst. ex A. Rich.) Stapf. or Urochloa brizantha (Hochst. ex A. Rich.) R. D. (beard grass, BRABR), Brachiaria platyphylla (Groseb.) Nash or Urochloa platyphylla (Nash) R. D. Webster (broadleaf signalgrass, BRAPP), Brachiaria plantaginea (Link) Hitchc. or Urochloa plantaginea (Link) R. D. Webster (alexandergrass, BRAPL), Cenchrus echinatus L. (southern sandbur, CENEC), Digitaria horizontalis Willd. (Jamaican crabgrass, DIGHO), Digitaria insularis (L.) Mez ex Ekman (sourgrass, TRCIN), Digitaria sanguinalis (L.) Scop. (large crabgrass, DIGSA), Echinochloa crus-galli (L.) P. Beauv. (barnyardgrass, ECHCG), Echinochloa colonum (L.) Link (junglerice, ECHCO), Eleusine indica (L.) Gaertn. (goosegrass, ELEIN), Lolium multiflorum Lam. (Italian ryegrass, LOLMU), Panicum dichotomiflorum Michx. (fall panicum, PANDI), Panicum miliaceum L. (wild-proso millet, PANMI), Setaria faberi Herrm. (giant foxtail, SETFA), Setaria viridis (L.) Beauv. (green foxtail, SETVI), Sorghum halepense (L.) Pers. (Johnsongrass, SORHA), Sorghum bicolor (L.) Moench ssp. Arundinaceum (shattercane, SORVU), Cyperus esculentus L. (yellow nutsedge, CYPES), Cyperus rotundus L. (purple nutsedge, CYPRO), Abutilon theophrasti Medik. (velvetleaf, ABUTH), Amaranthus species (pigweeds and amaranths, AMASS), Ambrosia artemisiifolia L. (common ragweed, AMBEL), Ambrosia psilostachya DC. (western ragweed, AMBPS), Ambrosia trifida L. (giant ragweed, AMBTR), Anoda cristata (L.) Schlecht. (spurred anoda, ANVCR), Asclepias syriaca L. (common milkweed, ASCSY), Bidens pilosa L. (hairy beggarticks, BIDPI), Borreria species (BOISS), Borreria alata (Aubl.) DC. or Spermacoce alata Aubl. (broadleaf buttonweed, BOILF), Spermacose latifolia (broadleaved button weed, BOILF), Chenopodium album L. (common lambsquarters, CHEAL), Cirsium arvense (L.) Scop. (Canada thistle, CIRAR), Commelina benghalensis L. (tropical spiderwort, COMBE), Datura stramonium L. (jimsonweed, DATST), Daucus carota L. (wild carrot, DAUCA), Euphorbia heterophylla L. (wild poinsettia, EPHHL), Euphorbia hirta L. or Chamaesyce hirta (L.) Millsp. (garden spurge, EPHHI), Euphorbia dentata Michx. (toothed spurge, EPHDE), Erigeron bonariensis L. or Conyza bonariensis (L.) Cronq. (hairy fleabane, ERIBO), Erigeron canadensis L. (Canadian fleabane, ERICA), Conyza sumatrensis (Retz.) E. H. Walker (tall fleabane, ERIFL), Helianthus annuus L. (common sunflower, HELAN), Jacquemontia tamnifolia (L.) Griseb. (smallflower morningglory, IAQTA), Ipomoea hederacea (L.) Jacq. (ivyleaf morningglory, IPOHE), Ipomoea lacunosa L. (white morningglory, IPOLA), Lactuca serriola L./Torn. (prickly lettuce, LACSE), Portulaca oleracea L. (common purslane, POROL), Richardia species (pusley, RCHSS), Sida species (sida, SIDSS), Sida spinosa L. (prickly sida, SIDSP), Sinapis arvensis L. (wild mustard, SINAR), Solanum ptychanthum Dunal (eastern black nightshade, SOLPT), Tridax procumbens L. (coat buttons, TRQPR) or Xanthium strumarium L. (common cocklebur, XANST).

In some aspects, the methods provided herein are utilized to control undesirable vegetation in turf. In certain aspects, the undesirable vegetation is Bellis perennis L. (English daisy, BELPE), Cyperus esculentus L. (yellow nutsedge, CYPES), Cyperus species (CYPSS), Digitaria sanguinalis (L.) Scop. (large crabgrass, DIGSA), Diodia virginiana L. (Virginia buttonweed, DIQVI), Euphorbia species (spurge, EPHSS), Glechoma hederacea L. (ground ivy, GLEHE), Hydrocotyle umbellata L. (dollarweed, HYDUM), Kyllinga species (kyllinga, KYLSS), Lamium amplexicaule L. (henbit, LAMAM), Murdannia nudiflora (L.) Brenan (doveweed, MUDNU), Oxalis species (woodsorrel, OXASS), Plantago major L. (broadleaf plantain, PLAMA), Plantago lanceolata L. (buckhorn/narrowleaf plantain, PLALA), Phyllanthus urinaria L. (chamberbitter, PYLTE), Rumex obtusifolius L. (broadleaf dock, RUMOB), Stachys floridana Shuttlew. (Florida betony, STAFL), Stellaria media (L.) Vill. (common chickweed, STEME), Taraxacum officinale G. H. Weber ex Wiggers (dandelion, TAROF), Trifolium repens L. (white clover, TRFRE), or Viola species (wild violet, VIOSS).

In some aspects, the compositions and methods provided herein are utilized to control undesirable vegetation, including grass, broadleaf and sedge weeds. In certain aspects, the compositions and methods provided herein are utilized to control undesirable vegetation including but not limited to Alopecurus, Avena, Centaurea, Cyperus, Digitaria, Echinochloa, Ipomoea, Leptochloa and Sonchus.

In some aspects, the combination of a compound of Formula (I) or formulation hereof may be used to control Abutilon theophrasti Medik. (velvetleaf, ABUTH), Amaranthus rudis Sauer, (common waterhemp, AMATA), Chenopodium album L. (common lambsquarters, CHEAL), Polygonum convolvulus L. (wild buckwheat, POLCO) and Sinapis arvensis L. (wild mustard, SINAR).

The compounds of Formula (I) or agriculturally acceptable formulation of the same may be used to control herbicide resistant or tolerant weeds. Exemplary resistant or tolerant weeds include, but are not limited to, biotypes resistant or tolerant to acetolactate synthase (ALS) or acetohydroxy acid synthase (AHAS) inhibitors, (e.g., imidazolinones, sulfonylureas, pyrimidinylthiobenzoates, triazolopyrimidines, and sulfonylaminocarbonyltriazolinones), photosystem II inhibitors (e.g., phenylcarbamates, pyridazinones, triazines, triazinones, uracils, amides, ureas, benzothiadiazinones, nitriles, phenylpyridazines), acetyl COA carboxylase (ACCase) inhibitors, (e.g., aryloxyphenoxypropionates, cyclohexanediones, phenylpyrazolines), synthetic auxins (e.g., benzoic acids, phenoxycarboxylic acids, pyridine carboxylic acids, quinoline carboxylic acids), auxin transport inhibitors (e.g., phthalamates, semicarbazones), photosystem I inhibitors (e.g., bipyridyliums), 5-enolpyruvylshikimate-3-phosphate (EPSP) synthase inhibitors (e.g., glyphosate), glutamine synthetase inhibitors (e.g., glufosinate, bialafos), microtubule assembly inhibitors (e.g., benzamides, benzoic acids, dinitroanilines, phosphoramidates, pyridines), mitosis inhibitors (e.g., carbamates), very long chain fatty acid (VLCFA) inhibitors (e.g., acetamides, chloroacetamides, oxyacetamides, tetrazolinones), fatty acid and lipid synthesis inhibitors (e.g., phosphorodithioates, thiocarbamates, benzofuranes, chlorocarbonic acids), protoporphyrinogen oxidase (PPO) inhibitors (e.g., diphenylethers, N-phenylphthalimides, oxadiazoles, oxazolidinediones, phenylpyrazoles, pyrimidindiones, thiadiazoles, triazolinones), carotenoid biosynthesis inhibitors (e.g., clomazone, amitrole, aclonifen), phytoene desaturase (PDS) inhibitors (e.g., amides, anilidex, furanones, phenoxybutan-amides, pyridiazinones, pyridines), 4-hydroxyphenyl-pyruvate-dioxygenase (HPPD) inhibitors (e.g., callistemones, isoxazoles, pyrazoles, triketones), cellulose biosynthesis inhibitors (e.g., nitriles, benzamides, quinclorac, triazolocarboxamides), herbicides with multiple modes-of-action such as quinclorac, and unclassified herbicides such as arylaminopropionic acids, difenzoquat, endothall, and organoarsenicals. Exemplary resistant or tolerant weeds include, but are not limited to, biotypes with resistance or tolerance to multiple herbicides, biotypes with resistance or tolerance to multiple chemical classes, biotypes with resistance or tolerance to multiple herbicide modes-of-action, and biotypes with multiple resistance or tolerance mechanisms (e.g., target site resistance or metabolic resistance).

With respect to the methods for treating crop fields to reduce or eliminate the growth of unwanted plants, these methods may comprise contacting the undesirable vegetation or locus thereof or applying to the soil or water to prevent the emergence or growth of vegetation or a composition described herein. In some aspects, the composition is applied at an application rate of from about 1 gram of acid equivalent per hectare (g ae/ha) to about 400 grams active ingredient per hectare (gai/ha) based on the total amount of active ingredients in the composition. In certain aspects, the composition is applied at an application rate of from about 4 grams acid equivalent per hectare (g ae/ha) to about 400 g ae/ha based on the total amount of active ingredients in the composition.

With respect to the methods, in certain aspects, the methods comprise contacting the undesirable vegetation or locus thereof or applying to the soil or water to prevent the emergence or growth of vegetation or a composition described herein. In some aspects, the composition is applied at an application rate sufficient to control, inhibit, or promote the growth of at least one plant. In some aspects the compound is applied, based on the total amount of active ingredients in the composition, in at least one of the following ranges: of from: about 1 gram to about 1,000 grams of acid equivalent per hectare (g ae/ha); in some aspects the composition is applied at an application rate of between about 1 gram to about 500 grams g ae/ha; in some aspects composition is applied at an application rate of between about 1 gram to about 250 grams g ae/ha; composition is applied at an application rate of between about 1 gram to about 140 grams g ae/h; in some aspects the composition is applied at an application rate of between about 1 gram to about 70 grams g ae/ha. In some aspects, the methods comprise contacting the undesirable vegetation or locus thereof or applying to the soil or water to prevent the emergence or growth of vegetation.

In some aspects, the concentration of the active ingredients in the compositions described herein is from about 0.0005 to 98 percent by weight. In some aspects, the concentration is from about 0.0006 to 90 percent by weight. In compositions designed to be employed as concentrates, the active ingredients, in certain aspects, are present in a concentration from about 0.1 to 98 weight percent, and in certain aspects about 0.5 to 90 weight percent. Such compositions are, in certain aspects, diluted with an inert carrier, such as water, before application. The diluted compositions usually applied to weeds or the locus of weeds contain, in certain aspects, about 0.0006 to 3.0 weight percent active ingredient and in certain aspects contain about 0.01 to 1.0 weight percent.

The present compositions can be applied to weeds or their locus by almost any conventional means of application, including but not limited to the use of aerial dusters, sprayers, and granule applicators. Under some circumstances these compounds and formulations thereof may be added to irrigation or paddy water, which is then used to treat plants, soil, and other surfaces.

The described aspects and following examples are for illustrative purposes and are not intended to limit the scope of the claims. Other modifications, uses, or combinations with respect to the compositions described herein will be apparent to a person of ordinary skill in the art without departing from the spirit and scope of the claimed subject matter.

Exemplary molecules within the aforementioned formulas according to various aspects may include any of the compounds exemplified below in Table 1.

TABLE 1
Pesticidal Compounds

| No. | Grp | Compound |
|---|---|---|
| A1 | A | 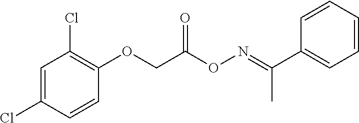 [SMILES: C/C(=N\OC(=O)COc1ccc(Cl)cc1Cl)c1ccccc1] |
| A2 | A | 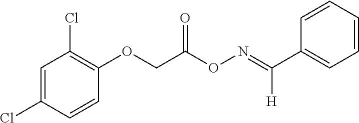 [SMILES: O=C(COc1ccc(Cl)cc1Cl)O/N=C/c1ccccc1] |
| A3 | A | 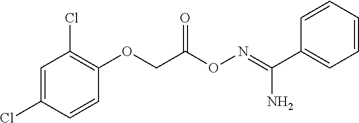 [SMILES: N/C(=N\OC(=O)COc1ccc(Cl)cc1Cl)c1ccccc1] |
| A4 | A | 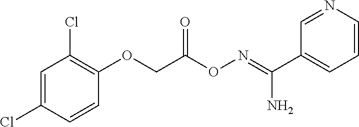 [SMILES: N/C(=N\OC(=O)COc1ccc(Cl)cc1Cl)c1cccnc1] |
| A5 | A | 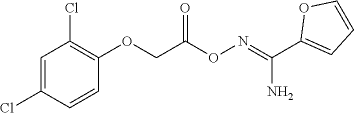 [SMILES: N/C(=N\OC(=O)COc1ccc(Cl)cc1Cl)c1ccco1] |
| A6 | A | 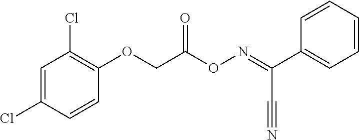 [SMILES: N#C/C(=N\OC(=O)COc1ccc(Cl)cc1Cl)c1ccccc1] |
| A7 | A | 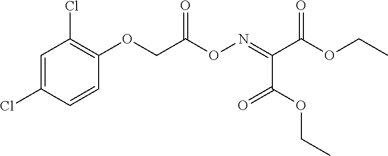 [SMILES: CCOC(=O)C(=NOC(=O)COc1ccc(Cl)cc1Cl)C(=O)OCC] |
| A8 | A | 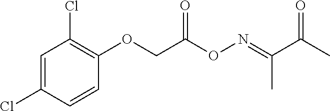 [SMILES: CC(=O)/C(C)=N/OC(=O)COc1ccc(Cl)cc1Cl] |
| A9 | A | 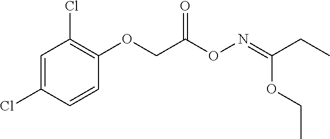 [SMILES: CCO/C(CC)=N\OC(=O)COc1ccc(Cl)cc1Cl] |
| A10 | A | 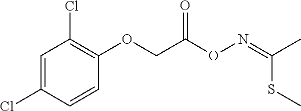 [SMILES: CS/C(C)=N\OC(=O)COc1ccc(Cl)cc1Cl] |
| A11 | A | 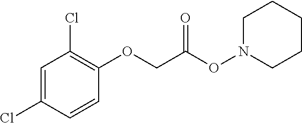 [SMILES: O=C(COc1ccc(Cl)cc1Cl)ON1CCCCC1] |
| A12 | A | 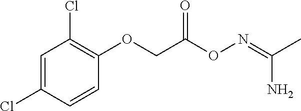 [SMILES: C/C(N)=N/OC(=O)COc1ccc(Cl)cc1Cl] |
| A13 | A | 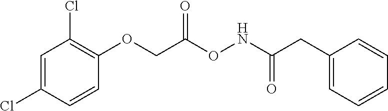 [SMILES: O=C(Cc1ccccc1)NOC(=O)COc1ccc(Cl)cc1Cl] |
| A14 | A | 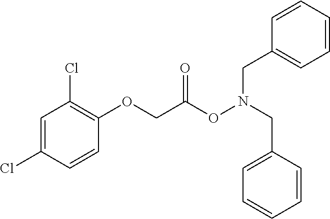 [SMILES: O=C(COc1ccc(Cl)cc1Cl)ON(Cc1ccccc1)Cc1ccccc1] |
| A15 | A | 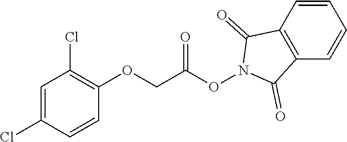 [SMILES: O=C(COc1ccc(Cl)cc1Cl)ON1C(=O)c2ccccc2C1=O] |
| A16 | A | 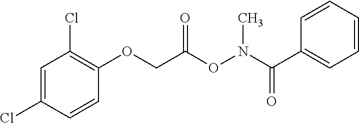 [SMILES: CN(OC(=O)COc1ccc(Cl)cc1Cl)C(=O)c1ccccc1] |
| A17 | A | 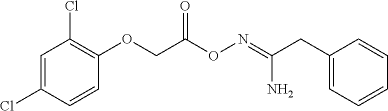 [SMILES: N/C(Cc1ccccc1)=N\OC(=O)COc1ccc(Cl)cc1Cl] |
| A18 | A | 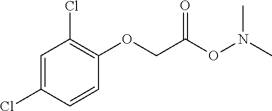 [SMILES: CN(C)OC(=O)COc1ccc(Cl)cc1Cl] |
| A19 | A | 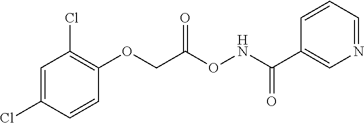 [SMILES: O=C(COc1ccc(Cl)cc1Cl)ONC(=O)c1cccnc1] |
| A20 | A | 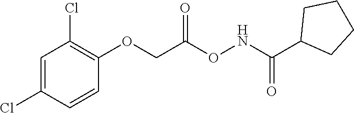 [SMILES: O=C(COc1ccc(Cl)cc1Cl)ONC(=O)C1CCCC1] |
| A21 | A | 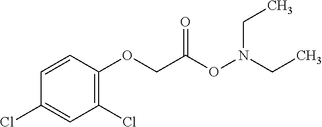 [SMILES: CCN(CC)OC(=O)COc1ccc(Cl)cc1Cl] |
| A22 | A | 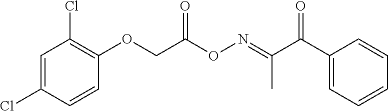 [SMILES: C/C(=N\OC(=O)COc1ccc(Cl)cc1Cl)C(=O)c1ccccc1] |
| A23 | A | 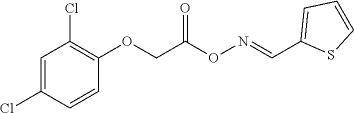 [SMILES: O=C(COc1ccc(Cl)cc1Cl)O/N=C/c1cccs1] |
| A24 | A | 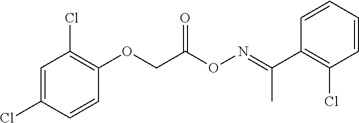 [SMILES: C/C(=N\OC(=O)COc1ccc(Cl)cc1Cl)c1ccccc1Cl] |
| A25 | A | 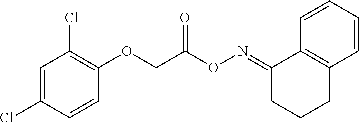 [SMILES: O=C(COc1ccc(Cl)cc1Cl)O/N=C1\CCCc2ccccc21] |
| A26 | A | 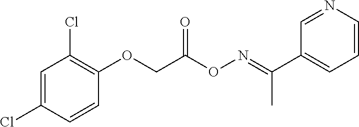 [SMILES: C/C(=N\OC(=O)COc1ccc(Cl)cc1Cl)c1cccnc1] |
| A27 | A | 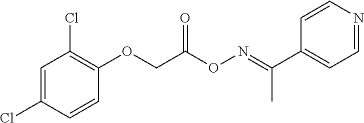 [SMILES: C/C(=N\OC(=O)COc1ccc(Cl)cc1Cl)c1ccncc1] |
| A28 | A | 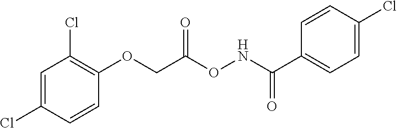 [SMILES: O=C(COc1ccc(Cl)cc1Cl)ONC(=O)c1ccc(Cl)cc1] |
| A29 | A | 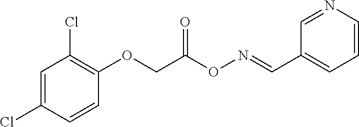 [SMILES: O=C(COc1ccc(Cl)cc1Cl)O/N=C/c1cccnc1] |
| A30 | A | 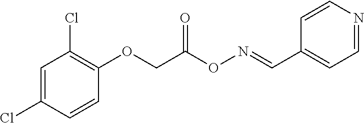 [SMILES: O=C(COc1ccc(Cl)cc1Cl)O/N=C/c1ccncc1] |
| A31 | A | 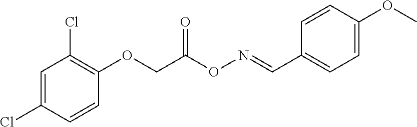 [SMILES: COc1ccc(/C=N/OC(=O)COc2ccc(Cl)cc2Cl)cc1] |
| A32 | A | 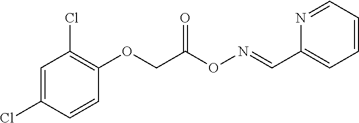 [SMILES: O=C(COc1ccc(Cl)cc1Cl)O/N=C/c1ccccn1] |
| A33 | A | 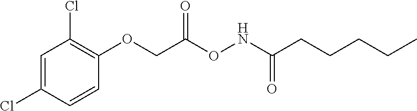 [SMILES: CCCCCC(=O)NOC(=O)COc1ccc(Cl)cc1Cl] |
| A34 | A | 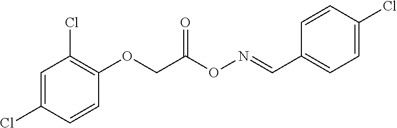 [SMILES: O=C(COc1ccc(Cl)cc1Cl)O/N=C/c1ccc(Cl)cc1] |
| A35 | A | 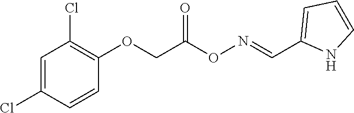 [SMILES: O=C(COc1ccc(Cl)cc1Cl)O/N=C/c1ccc[nH]1] |
| A36 | A | 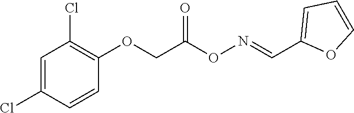 [SMILES: O=C(COc1ccc(Cl)cc1Cl)O/N=C/c1ccco1] |
| A37 | A | 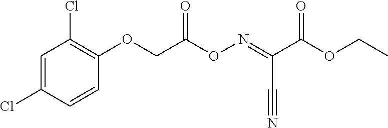 [SMILES: CCOC(=O)/C(C#N)=N/OC(=O)COc1ccc(Cl)cc1Cl] |
| A38 | A | 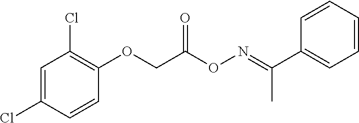 [SMILES: C/C(=N\OC(=O)COc1ccc(Cl)cc1Cl)c1ccccc1] |
| A39 | A | 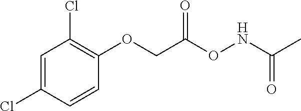 [SMILES: CC(=O)NOC(=O)COc1ccc(Cl)cc1Cl] |
| A40 | A | 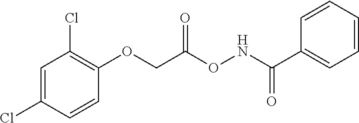 [SMILES: O=C(COc1ccc(Cl)cc1Cl)ONC(=O)c1ccccc1] |
| A41 | A | 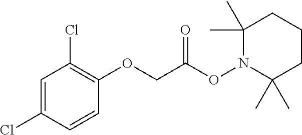 [SMILES: CC1(C)CCCC(C)(C)N1OC(=O)COc1ccc(Cl)cc1Cl] |
| B1 | B | 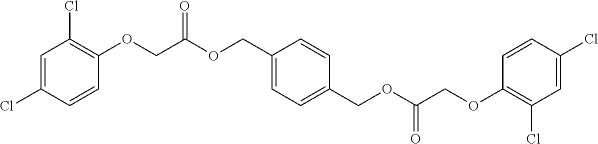 [SMILES: O=C(COc1ccc(Cl)cc1Cl)OCc1ccc(COC(=O)COc2ccc(Cl)cc2Cl)cc1] |
| B2 | B | 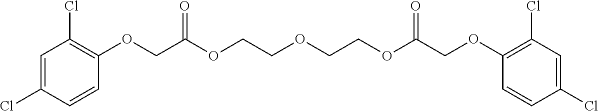 [SMILES: O=C(COc1ccc(Cl)cc1Cl)OCCOCCOC(=O)COc1ccc(Cl)cc1Cl] |
| B3 | B | 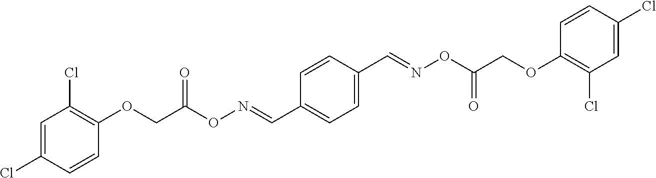 [SMILES: O=C(COc1ccc(Cl)cc1Cl)O/N=C/c1ccc(/C=N/OC(=O)COc2ccc(Cl)cc2Cl)cc1] |
| B4 | B | 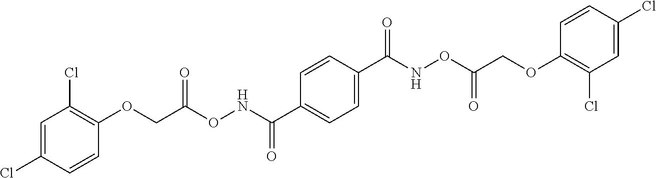 [SMILES: O=C(COc1ccc(Cl)cc1Cl)ONC(=O)c1ccc(C(=O)NOC(=O)COc2ccc(Cl)cc2Cl)cc1] |
| B5 | B | 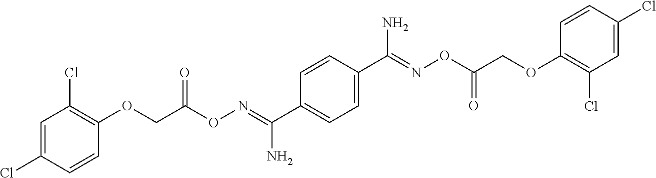 [SMILES: N/C(=N\OC(=O)COc1ccc(Cl)cc1Cl)c1ccc(/C(N)=N/OC(=O)COc2ccc(Cl)cc2Cl)cc1] |
| B6 | B | 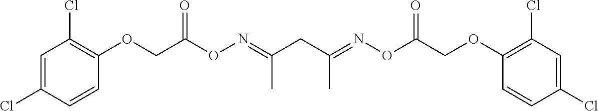 [SMILES: C/C(C/C(C)=N/OC(=O)COc1ccc(Cl)cc1Cl)=N\OC(=O)COc1ccc(Cl)cc1Cl] |
| B7 | B | 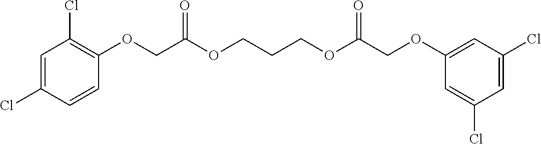 [SMILES: O=C(COc1cc(Cl)cc(Cl)c1)OCCCOC(=O)COc1ccc(Cl)cc1Cl] |
| B8 | B | 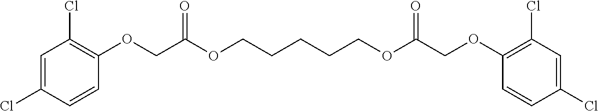 [SMILES: O=C(COc1ccc(Cl)cc1Cl)OCCCCCOC(=O)COc1ccc(Cl)cc1Cl] |
| B9 | B | 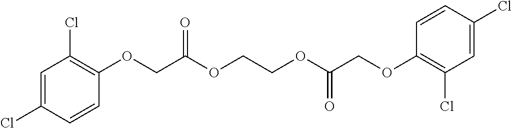 [SMILES: O=C(COc1ccc(Cl)cc1Cl)OCCOC(=O)COc1ccc(Cl)cc1Cl] |
| B10 | B | 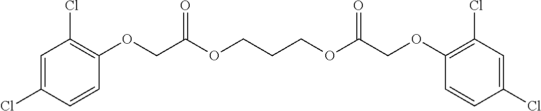 [SMILES: O=C(COc1ccc(Cl)cc1Cl)OCCCOC(=O)COc1ccc(Cl)cc1Cl] |
| B11 | B | 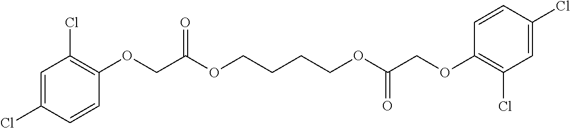 [SMILES: O=C(COc1ccc(Cl)cc1Cl)OCCCCOC(=O)COc1ccc(Cl)cc1Cl] |
| B12 | B | 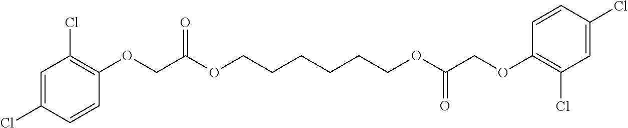 [SMILES: O=C(COc1ccc(Cl)cc1Cl)OCCCCCCOC(=O)COc1ccc(Cl)cc1Cl] |
| B13 | B | 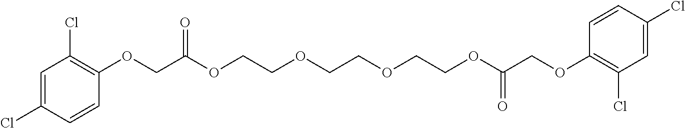 [SMILES: O=C(COc1ccc(Cl)cc1Cl)OCCOCCOCCOC(=O)COc1ccc(Cl)cc1Cl] |
| B14 | B | 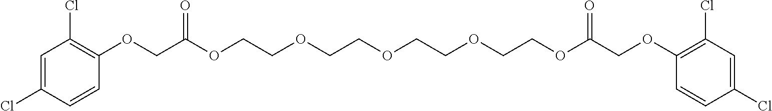 [SMILES: O=C(COc1ccc(Cl)cc1Cl)OCCOCCOCCOCCOC(=O)COc1ccc(Cl)cc1Cl] |
| B15 | B | 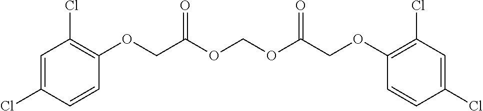 [SMILES: O=C(COc1ccc(Cl)cc1Cl)OCOC(=O)COc1ccc(Cl)cc1Cl] |
| C1 | C | 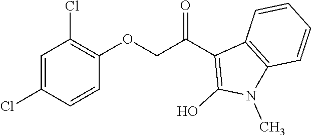 [SMILES: Cn1c(O)c(C(=O)COc2ccc(Cl)cc2Cl)c2ccccc21] |
| D1 | D | 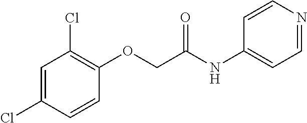 [SMILES: O=C(COc1ccc(Cl)cc1Cl)Nc1ccncc1] |
| D2 | D | 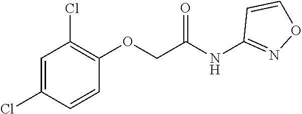 [SMILES: O=C(COc1ccc(Cl)cc1Cl)Nc1ccon1] |
| D3 | D | 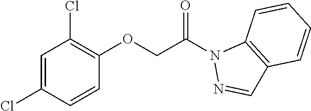 [SMILES: O=C(COc1ccc(Cl)cc1Cl)n1ncc2ccccc21] |
| D4 | D | 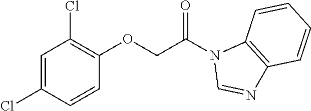 [SMILES: O=C(COc1ccc(Cl)cc1Cl)n1cnc2ccccc21] |
| D5 | D | 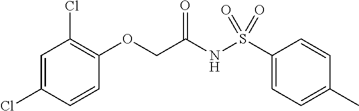 [SMILES: Cc1ccc(S(=O)(=O)NC(=O)COc2ccc(Cl)cc2Cl)cc1] |
| D6 | D | 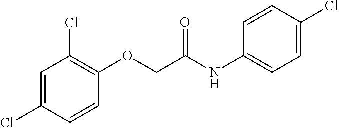 [SMILES: O=C(COc1ccc(Cl)cc1Cl)Nc1ccc(Cl)cc1] |
| D7 | D | 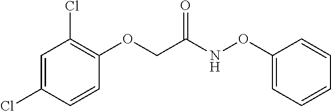 [SMILES: O=C(COc1ccc(Cl)cc1Cl)NOc1ccccc1] |
| D8 | D | 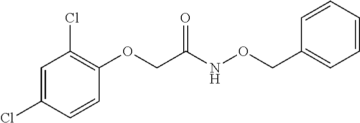 [SMILES: O=C(COc1ccc(Cl)cc1Cl)NOCc1ccccc1] |
| D9 | D | 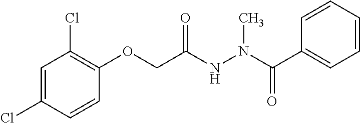 [SMILES: CN(NC(=O)COc1ccc(Cl)cc1Cl)C(=O)c1ccccc1] |
| D10 | D | 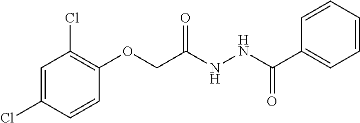 [SMILES: O=C(COc1ccc(Cl)cc1Cl)NNC(=O)c1ccccc1] |
| D11 | D | 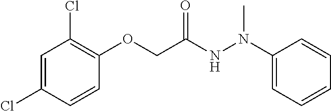 [SMILES: CN(NC(=O)COc1ccc(Cl)cc1Cl)c1ccccc1] |
| D12 | D | 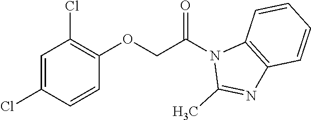 [SMILES: Cc1nc2ccccc2n1C(=O)COc1ccc(Cl)cc1Cl] |
| D13 | D | 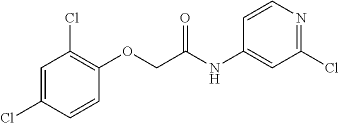 [SMILES: O=C(COc1ccc(Cl)cc1Cl)Nc1ccnc(Cl)c1] |
| D14 | D | 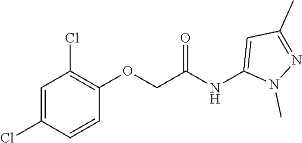 [SMILES: Cc1cc(NC(=O)COc2ccc(Cl)cc2Cl)n(C)n1] |
| D15 | D | 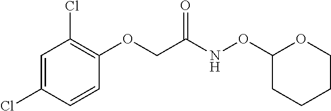 [SMILES: O=C(COc1ccc(Cl)cc1Cl)NOC1CCCCO1] |
| D16 | D | 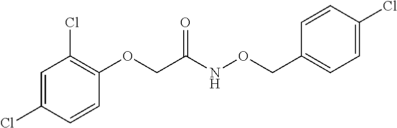 [SMILES: O=C(COc1ccc(Cl)cc1Cl)NOCc1ccc(Cl)cc1] |
| D17 | D | 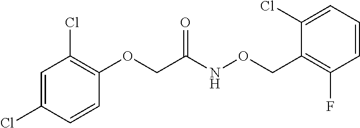 [SMILES: O=C(COc1ccc(Cl)cc1Cl)NOCc1c(F)cccc1Cl] |
| D18 | D | 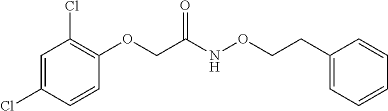 [SMILES: O=C(COc1ccc(Cl)cc1Cl)NOCCc1ccccc1] |
| D19 | D | 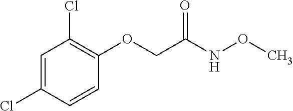 |
| D20 | D | 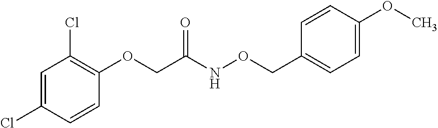 [SMILES: COc1ccc(CONC(=O)COc2ccc(Cl)cc2Cl)cc1] |
| D21 | D | 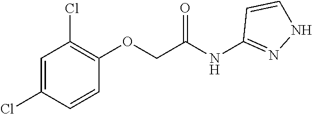 [SMILES: O=C(COc1ccc(Cl)cc1Cl)Nc1cc[nH]n1] |
| D22 | D | 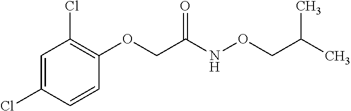 [SMILES: CC(C)CONC(=O)COc1ccc(Cl)cc1Cl] |
| D23 | D | 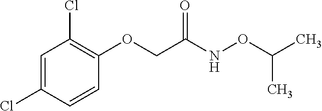 [SMILES: CC(C)ONC(=O)COc1ccc(Cl)cc1Cl] |
| D24 | D | 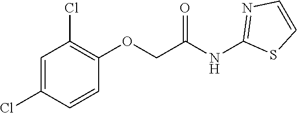 [SMILES: O=C(COc1ccc(Cl)cc1Cl)Nc1nccs1] |
| D25 | D | 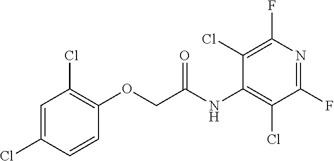 [SMILES: O=C(COc1ccc(Cl)cc1Cl)Nc1c(Cl)c(F)nc(F)c1Cl] |
| D26 | D | 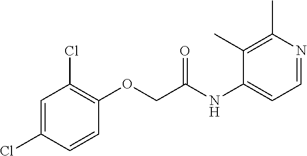 [SMILES: Cc1nccc(NC(=O)COc2ccc(Cl)cc2Cl)c1C] |
| D27 | D | 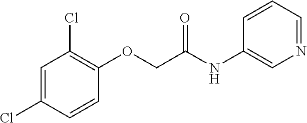 [SMILES: O=C(COc1ccc(Cl)cc1Cl)Nc1cccnc1] |
| D28 | D | 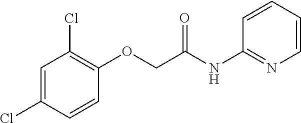 [SMILES: O=C(COc1ccc(Cl)cc1Cl)Nc1ccccn1] |
| D29 | D | 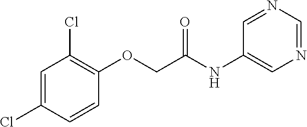 [SMILES: O=C(COc1ccc(Cl)cc1Cl)Nc1cncnc1] |
| D30 | D | 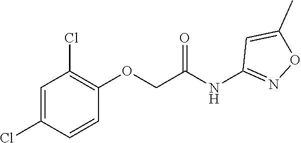 [SMILES: Cc1cc(NC(=O)COc2ccc(Cl)cc2Cl)no1] |
| D31 | D | 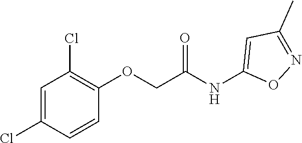 [SMILES: Cc1cc(NC(=O)COc2ccc(Cl)cc2Cl)on1] |
| D32 | D | 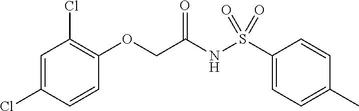 [SMILES: Cc1ccc(S(=O)(=O)NC(=O)COc2ccc(Cl)cc2Cl)cc1] |
| D33 | D | 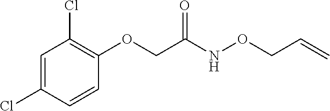 [SMILES: C=CCONC(=O)COc1ccc(Cl)cc1Cl] |
| E1 | E | 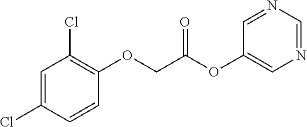 [SMILES: O=C(COc1ccc(Cl)cc1Cl)Oc1cncnc1] |
| E2 | E | 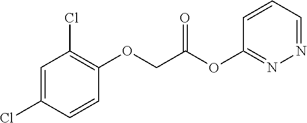 [SMILES: O=C(COc1ccc(Cl)cc1Cl)Oc1cccnn1] |
| E3 | E | 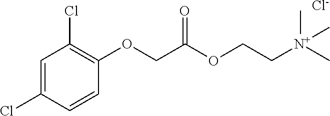 [SMILES: [Cl-] ; C[N+](C)(C)CCOC(=O)COc1ccc(Cl)cc1Cl] |
| E4 | E | 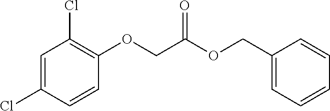 [SMILES: O=C(COc1ccc(Cl)cc1Cl)OCc1ccccc1] |
| E5 | E | 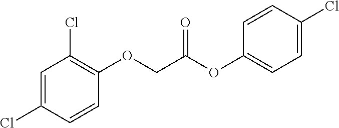 [SMILES: O=C(COc1ccc(Cl)cc1Cl)Oc1ccc(Cl)cc1] |
| E6 | E | 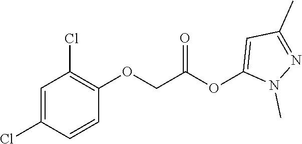 [SMILES: Cc1cc(OC(=O)COc2ccc(Cl)cc2Cl)n(C)n1] |
| E7 | E | 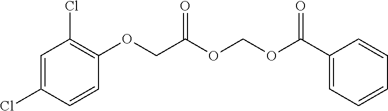 [SMILES: O=C(COc1ccc(Cl)cc1Cl)OCOC(=O)c1ccccc1] |
| E8 | E | 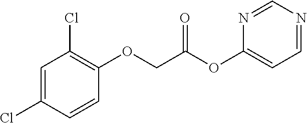 [SMILES: O=C(COc1ccc(Cl)cc1Cl)Oc1ccncn1] |
| E9 | E | 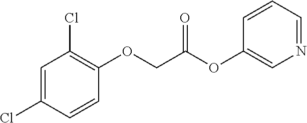 [SMILES: O=C(COc1ccc(Cl)cc1Cl)Oc1cccnc1] |
| E10 | E | 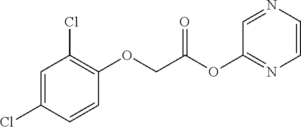 [SMILES: O=C(COc1ccc(Cl)cc1Cl)Oc1cnccn1] |
| E11 | E | 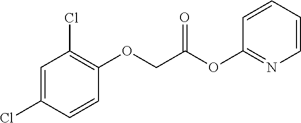 [SMILES: O=C(COc1ccc(Cl)cc1Cl)Oc1ccccn1] |
| E12 | E | 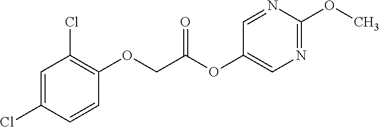 [SMILES: COc1ncc(OC(=O)COc2ccc(Cl)cc2Cl)cn1] |
| E13 | E | 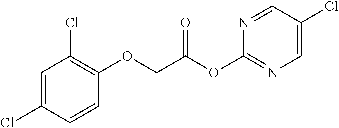 [SMILES: O=C(COc1ccc(Cl)cc1Cl)Oc1ncc(Cl)cn1] |
| E14 | E | 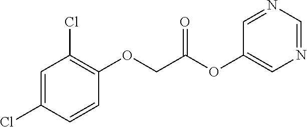 [SMILES: O=C(COc1ccc(Cl)cc1Cl)Oc1cncnc1] |
| E15 | E | 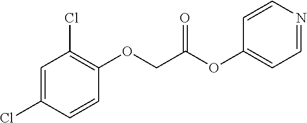 [SMILES: O=C(COc1ccc(Cl)cc1Cl)Oc1ccncc1] |
| E16 | E | 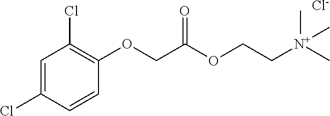 [SMILES: [Cl-] ; C[N+](C)(C)CCOC(=O)COc1ccc(Cl)cc1Cl] |
| E17 | E | 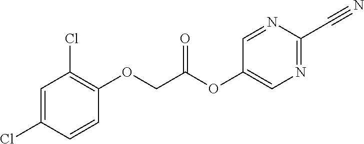 [SMILES: N#Cc1ncc(OC(=O)COc2ccc(Cl)cc2Cl)cn1] |
| E18 | E | 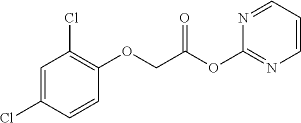 [SMILES: O=C(COc1ccc(Cl)cc1Cl)Oc1ncccn1] |
| E19 | E | 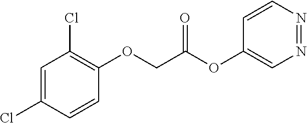 [SMILES: O=C(COc1ccc(Cl)cc1Cl)Oc1ccnnc1] |

### EXAMPLES

The following exemplary organic synthesis routes may be used to create some of the various compounds disclosed herein. While a person of ordinary skill will appreciate these exemplary organic synthesis routes, it is within the knowledge of one skilled in the art—with the benefit of the present disclosure—to utilize these routes as well as affect other organic synthesis routes whether or not explicitly described herein. Thus, the present disclosure would not be seen as limiting to one of ordinary skill and, after reading the present description, it may be apparent to one skilled in the relevant art(s) how synthesize various compounds described herein with alternate organic synthesis routes.

Furthermore, many of the inputs for the synthesis routes are commercially available. Thus, in some examples, the CAS Number is included for select inputs above the intermediate, for example, sometimes when commercially available.

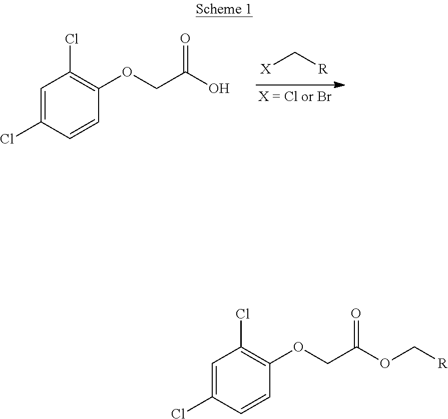

### Example S1-1 Benzyl 2-(2,4-dichlorophenoxy)acetate

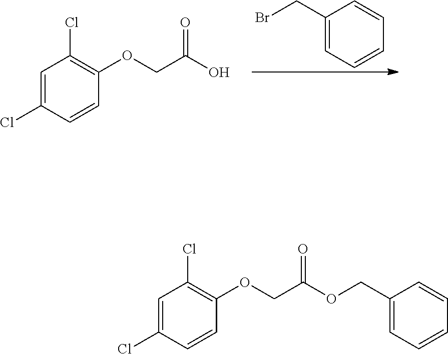 [SMILES: O=C(O)COc1ccc(Cl)cc1Cl ; BrCc1ccccc1 ; O=C(COc1ccc(Cl)cc1Cl)OCc1ccccc1]

(Bromomethyl)benzene (0.46 g, 2.71 mmol) in DMF (5 mL) was added to a mixture of K₂CO₃ in DMF (10 mL) at RT and stirred for 10 min. The mixture was cooled to 0° C., then 2,4-dichlorophenoxyacetic acid (0.5 g, 2.26 mmol) was added under an argon atmosphere and stirred at room temperature for 16 h. Upon completion of the reaction, water (50 mL) was added and the mixture extracted with EtOAc (2×100 mL). The combined organic extracts were washed with brine, dried over Na₂SO₄, filtered and concentrated in vacuo which gave the crude product. Purification by flash column chromatography (silica gel; gradient elution 0-30% EtOAc:hexane) gave the title compound E4 as an off-white solid (0.33 g, 47%). ¹H NMR (400 MHz, CDCl₃): δ 7.38 (d, J=2.5 Hz, 1H), 7.29 (m, 5H), 7.11 (dd, J=8.8, 2.5 Hz, 1H), 6.73 (d, J=8.8 Hz, 1H), 5.23 (s, 2H), 4.72 (s, 2H). LCMS: m/z=333.0.1 [M+Na]⁺.

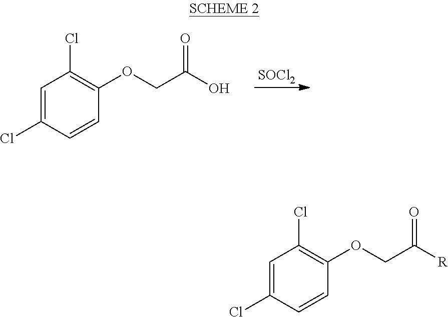

### Example S2-1-Compound 3:2-(2,4-Dichlorophenoxy)acetyl chloride

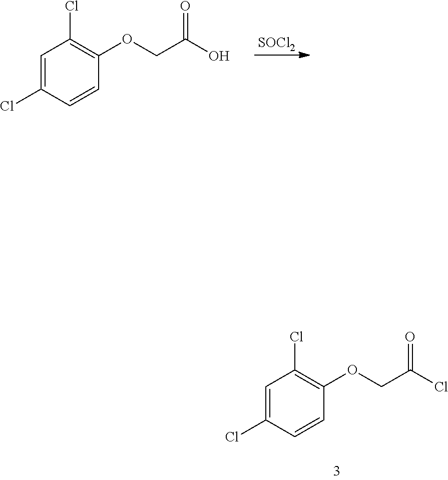

2,4-Dichlorophenoxyacetic acid (100 g, 0.452 mol) was dissolved in SOCl₂ (340 mL), and the reaction mixture was heated at 80° C. for 4 h. Completion of reaction was monitored by HPLC. Upon completion of the reaction, excess SOCl₂ was removed in vacuo which gave the title compound 3 as a pale-yellow oil (98.5 g, 90%). ¹H NMR (400 MHz, CDCl₃): δ 7.42 (d, J=2.5 Hz, 1H), 7.20 (dd, J=8.8, 2.5 Hz, 1H), 6.82 (d, J=8.8 Hz, 1H), 4.99 (s, 2H). LCMS: No molecular ion detected.

### Example S2-2 (2-(2,4-Dichlorophenoxy)acetoxy)methyl benzoate

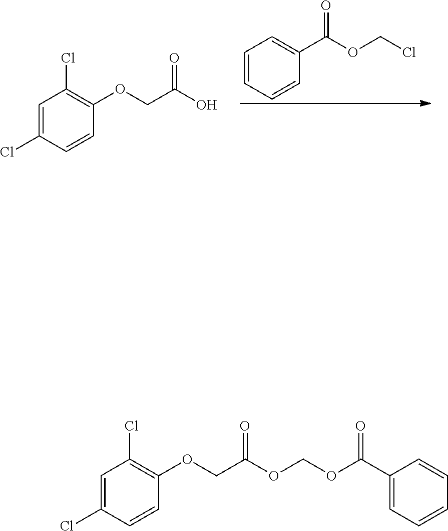 [SMILES: O=C(O)COc1ccc(Cl)cc1Cl ; O=C(OCCl)c1ccccc1 ; O=C(COc1ccc(Cl)cc1Cl)OCOC(=O)c1ccccc1]

To a solution of 2,4-dichlorophenoxyacetic acid (1 g, 4.52 mmol) in toluene (25 mL) was added Ag₂CO₃ (3.74 g, 13.56 mmol) and chloromethyl benzoate (2.31 g, 13.56 mmol) at RT. The mixture was stirred for 10 min at RT and was then heated to 80° C. for 5 h. Upon completion of the reaction, the mixture was concentrated in vacuo which gave the crude product. Purification by flash column chromatography (silica gel; gradient elution 0-30% EtOAc:hexane) gave the title compound E7 as a white solid (1 g, 63%). ¹H NMR (400 MHz, DMSO-d6): δ 7.95 (m, 2H), 7.68 (m, 1H), 7.53 (m, 3H), 7.29 (dd, J=8.9, 2.6 Hz, 1H), 7.14 (d, J=8.9 Hz, 1H), 6.05 (s, 2H), 5.08 (s, 2H). LCMS: m/z=376.97 [M+Na]⁺.

### Example S2-3 Methylene bis(2-(2,4-dichlorophenoxy)acetate)

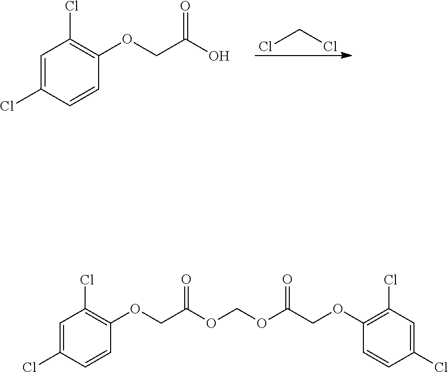

To a solution of 2,4-dichlorophenoxyacetic acid (0.44 g, 2 mmol) in 1:1 DMSO:DCM (14 mL) was added K₂CO₃ (0.28 g, 4 mmol) and the mixture stirred for 15 min at RT. The reaction mixture was then sealed and heated to 130° C. for 5 h. Upon completion of the reaction, water (50 mL) was added; the solid obtained was collected by filtration under suction and pulled dry which gave the title compound B15 as a white solid (1 g, 33%). ¹H NMR (400 MHz, DMSO-d6): δ 7.62 (dd, J=2.6, 1.2 Hz, 1H), 7.35 (dd, J=8.8, 2.6 Hz, 1H), 7.14 (d, J=9.0 Hz, 1H), 5.86 (s, 1H), 5.04 (s, 2H). LCMS: m/z=476.89 [M+Na]⁺.

### Example S2-4 1,4-Phenylenebis(methylene) bis(2-(2,4-dichlorophenoxy)acetate)

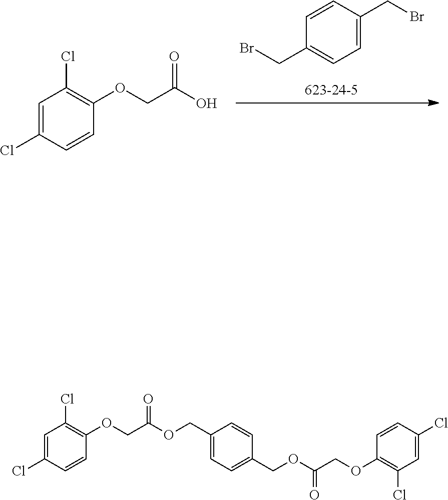 [SMILES: O=C(O)COc1ccc(Cl)cc1Cl ; BrCc1ccc(CBr)cc1 ; O=C(COc1ccc(Cl)cc1Cl)OCc1ccc(COC(=O)COc2ccc(Cl)cc2Cl)cc1]

To a solution of 2,4-dichlorophenoxyacetic acid (1 g, 4.52 mmol) in DMF (7 mL) was added K₂CO₃ (0.9 g, 6.78 mmol) and the mixture stirred for 10 min at RT. The mixture was cooled to 0° C., then a,a′-dibromo-p-xylene (0.6 g, 2.26 mmol) was added under an argon atmosphere and stirred at RT for 16 h. Upon completion of the reaction, water (100 mL) was added, and the mixture extracted with EtOAc (2×100 mL). The combined organic extracts were washed with brine, dried over Na₂SO₄, filtered and concentrated in vacuo which gave the crude product. Purification by flash column chromatography (silica gel; gradient elution 0-50% EtOAc:hexane) gave the title compound B1 as a white solid (0.5 g, 42%). ¹H NMR (400 MHz, DMSO-d6): δ 7.60 (d, J=2.6 Hz, 2H), 7.35 (d, J=15.0 Hz, 6H), 7.13 (d, J=8.9 Hz, 2H), 5.21 (s, 4H), 5.03 (s, 4H). LCMS: No molecular ion detected.

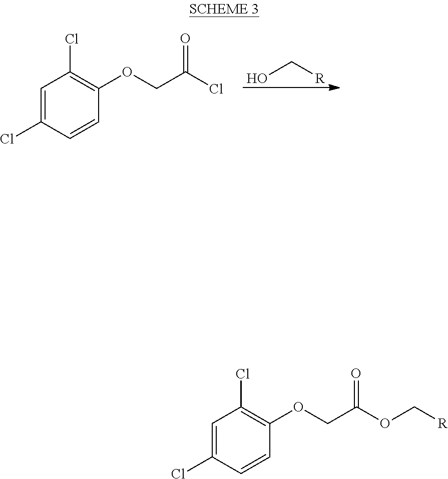

### Example S3-1 2-(2-(2,4-Dichlorophenoxy)acetoxy)-N, N, N-trimethylethanaminium chloride

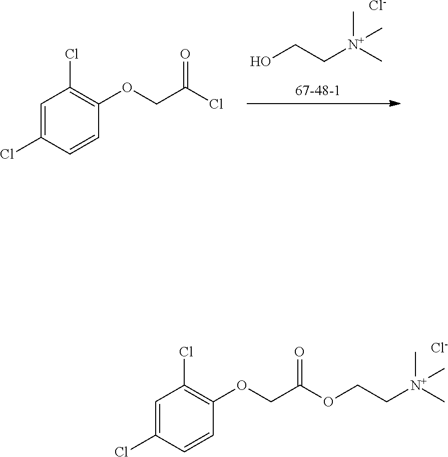

To a solution of 2-hydroxy-N, N, N-trimethylethanaminium chloride (1.46 g, 10.44 mmol) in CHCl₃ (20 mL) was added a solution of 2,4-dichlorophenoxyacetic 2,4-dichlorophenoxyacetyl chloride 3 (2.5 g, 10.44 mmol) in CHCl₃ (5 mL). The reaction mixture was heated at 45° C. for 16 h. Upon completion of the reaction, the RM was concentrated in vacuo which gave the crude product. Purification by flash column chromatography (silica gel; gradient elution 0-30% EtOAc:hexane) gave the title compound E3 as a white solid (0.7 g, 37%). ¹H NMR (400 MHz, DMSO-d6): δ 7.62 (d, J=2.6 Hz, 1H), 7.34 (m, 1H), 7.23 (d, J=9.0 Hz, 1H), 5.01 (s, 2H), 4.58 (s, 2H), 3.69 (m, 2H), 3.16 (s, 9H). LCMS: No molecular ion detected.

### Example S3-2 Oxybis(ethane-2,1-diyl) bis(2-(2,4-dichlorophenoxy)acetate)

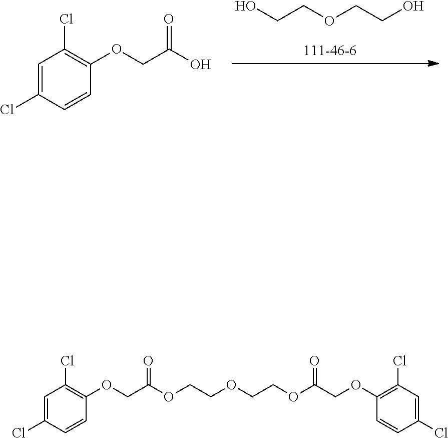 [SMILES: O=C(O)COc1ccc(Cl)cc1Cl ; OCCOCCO ; O=C(COc1ccc(Cl)cc1Cl)OCCOCCOC(=O)COc1ccc(Cl)cc1Cl]

To a solution of 2,2′-oxydiethanol (0.24 g, 2.26 mmol), 2,4-dichlorophenoxyacetic acid (1 g, 4.52 mmol) and TEA (2.2 mL g, 15.84 mmol) in DCM (15 mL) was added HATU (6.02 g, 15.84 mmol) and the resultant mixture stirred at RT for 16 h. Upon completion of the reaction, the RM was concentrated in vacuo which gave the crude product. Purification by flash column chromatography (silica gel; gradient elution 0-15% EtOAc:hexane) gave the title compound B2 as an orange oil (0.4 g, 35%). ¹H NMR (400 MHz, DMSO-d6): δ 7.59 (d, J=2.6 Hz, 1H), 7.34 (dd, J=8.9, 2.6 Hz, 1H), 7.10 (d, J=8.9 Hz, 1H), 4.97 (s, 2H), 4.12 (m, 2H), 3.48 (m, 2H). LCMS: No molecular ion detected.

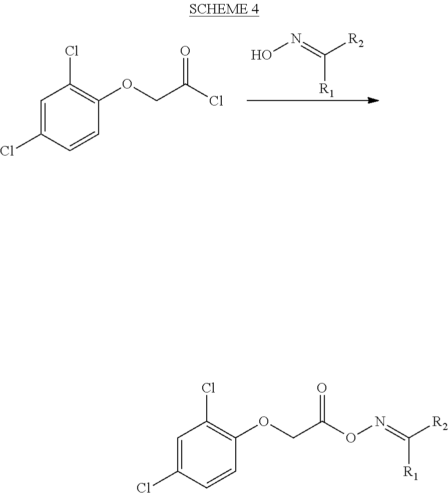

### Example S4-1 Acetophenone O-(2-(2,4-dichlorophenoxy)acetyl) oxime

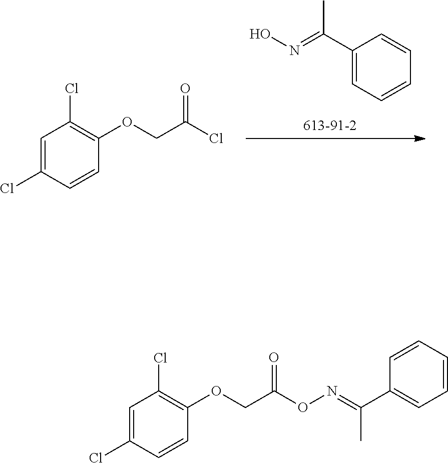

To a solution of acetophenone oxime (1 g, 7.40 mmol) in CHCl₃ (20 mL) was added NMM (2.25 g, 22.2 mmol) and the resultant mixture stirred at RT for 10 min. The mixture was cooled to 0° C., then 2,4-dichlorophenoxyacetyl chloride 3 (2.66 g, 11.1 mmol) was added under an argon atmosphere and stirred for 16 h at RT. Upon completion of the reaction, water (50 mL) was added, and the mixture extracted with EtOAc (2×100 mL). The combined organic extracts were washed with brine, dried over Na₂SO₄, filtered and concentrated in vacuo which gave the crude product. Purification by flash column chromatography (silica gel; gradient elution 0-20% EtOAc:hexane) gave the title compound A1 as a white solid (0.3 g, 11%). ¹H NMR (400 MHz, CDCl₃): δ 7.66 (m, 2H), 7.33 (m, 4H), 7.17 (dd, J=8.8, 2.6 Hz, 1H), 6.88 (d, J=8.8 Hz, 1H), 4.97 (s, 2H), 2.35 (s, 3H). LCMS: m/z=359.67 [M+Na]⁺.

### Example S4-2 Benzaldehyde O-(2-(2,4-dichlorophenoxy)acetyl) oxime

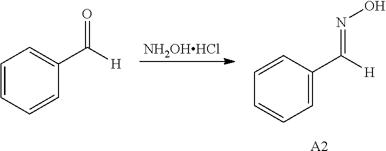

### Synthesis of Intermediate A2

To a solution of benzaldehyde (2 g, 18.8 mmol) in EtOH:H₂O (57 mL: 19 mL) was added KOAc (9.23 g, 94 mmol) and NH₂OH·HCl (4.71 g, 56.4 mmol) and the mixture stirred for 10 min at RT. The reaction mixture was heated to reflux for 5 h. Upon completion of the reaction, The RM was cooled to 0° C. and the solid obtained was filtered and dried in vacuo which gave Intermediate A2 as a white crystalline solid (1.1 g, 11%).

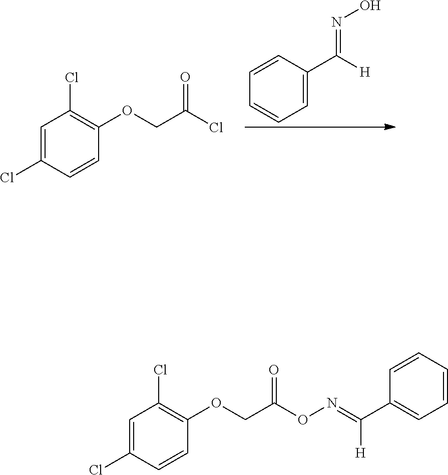

### Example A2 (E)-benzaldehyde O-(2-(2,4-dichlorophenoxy)acetyl) oxime

To a solution of Intermediate A2 (1 g, 8.26 mmol) in CHCl₃ (20 mL) was added NMM (2.51 g, 24.78 mmol) and the resultant mixture stirred for 10 min at RT. The mixture was cooled to 0° C., then 2,4-dichlorophenoxyacetyl chloride 3 (2.97 g, 12.39 mmol) was added under an argon atmosphere and the mixture was stirred for 16 h at RT. Upon completion of the reaction, water (100 mL) was added and the mixture was extracted with EtOAc (2×100 mL). The combined organic extracts were washed with brine, dried over Na₂SO₄, filtered and concentrated in vacuo which gave the crude product. Purification by flash column chromatography (silica gel; gradient elution 0-50% EtOAc:hexane) gave the title compound A2 as a white solid (0.3 g, 12%). ¹H NMR (400 MHz, DMSO-d6): δ 8.76 (s, 1H), 7.70 (m, 2H), 7.46 (m, 4H), 7.39 (dd, J=8.9, 2.6 Hz, 1H), 7.23 (d, J=8.9 Hz, 1H), 5.24 (s, 2H). LCMS: m/z=345.88 [M+Na]⁺.

### Example S4-3 N′-(2-(2,4-Dichlorophenoxy)acetoxy)benzimidamide

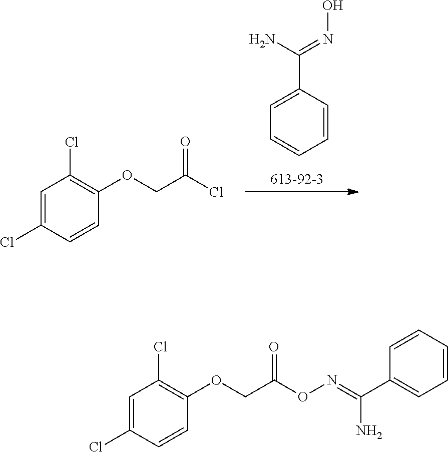 [SMILES: O=C(Cl)COc1ccc(Cl)cc1Cl ; N/C(=N\O)c1ccccc1 ; N/C(=N\OC(=O)COc1ccc(Cl)cc1Cl)c1ccccc1]

To a solution of N′-hydroxybenzimidamide (1 g, 0.73 mmol) in THF (20 mL) was added DIPEA (3.8 mL, 2.20 mmol) at 0° C. The mixture was stirred for 30 min and solution of 2,4-dichlorophenoxyacetyl chloride 3 (1.93 g, 0.80 mmol) in THF (5 mL) was added. Further the reaction was brought to RT and stirred for 15 h. Upon completion of the reaction, water (40 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-60% EtOAc:hexane) gave the title compound A3 as a pale brown solid (0.44 g, 18%). ¹H NMR (400 MHz, DMSO-d6): δ 7.77-7.68 (m, 2H), 7.62 (d, J=2.6 Hz, 1H), 7.57-7.33 (m, 4H), 7.17 (d, J=8.9 Hz, 1H), 6.99 (s, 2H), 5.16 (s, 2H). LCMS: m/z=338.88 [M+H]⁺.

### Example S4-4 N′-(2-(2,4-Dichlorophenoxy)acetoxy)nicotinimidamide

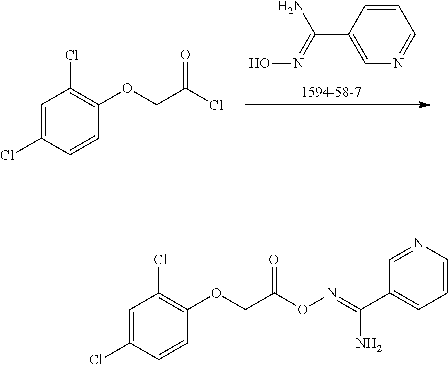 [SMILES: O=C(Cl)COc1ccc(Cl)cc1Cl ; N/C(=N\O)c1cccnc1 ; N/C(=N\OC(=O)COc1ccc(Cl)cc1Cl)c1cccnc1]

To a solution of N′-hydroxynicotinimidamide (1 g, 0.73 mmol) in THF (20 mL) was added DIPEA (3.8 mL, 2.20 mmol) at 0° C. The mixture was stirred for 30 min and solution of 2,4-dichlorophenoxyacetyl chloride 3 (1.92 g, 0.80 mmol) in THF (5 mL) was added. Further the reaction was brought to RT and stirred for 15 h. Upon completion of the reaction, water (40 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-60% EtOAc:hexane) gave the title compound A4 as a pale brown solid (1.18 g, 46%). ¹H NMR (400 MHz, DMSO-d6): δ 8.94-8.87 (m, 1H), 8.74-8.66 (m, 1H), 8.14-8.05 (m, 1H), 7.62 (d, J=2.6 Hz, 1H), 7.55-7.47 (m, 1H), 7.40 (dd, J=8.9, 2.6 Hz, 1H), 7.25-7.08 (m, 3H), 5.18 (s, 2H). LCMS: m/z=339.96 [M+1]⁺.

### Example S4-5 N′-(2-(2,4-Dichlorophenoxy)acetoxy)acetimidamide

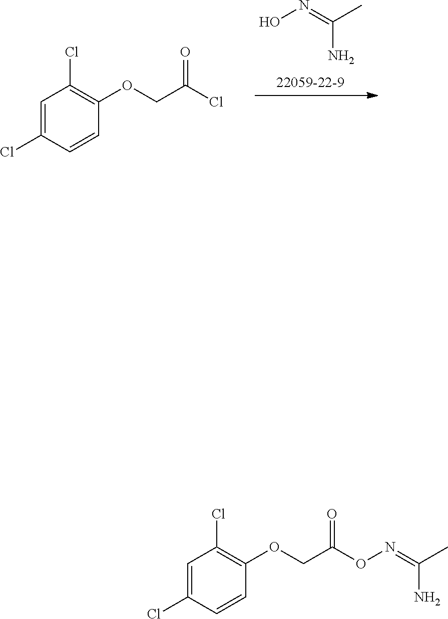

To a solution of N′-hydroxyacetimidamide (1 g, 1.34 mmol) in THF (20 mL) was added DIPEA (7.04 mL, 4.04 mmol) at 0° C. The mixture was stirred for 30 min and solution of 2,4-dichlorophenoxyacetyl chloride 3 (3.56 g, 1.48 mmol) in THF (5 mL) was added. Further the reaction was brought to RT and stirred for 15 h. Upon completion of the reaction, water (40 mL) was added, and the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-25% EtOAc:hexane) gave the title compound A12 as a pale-yellow solid (0.77 g, 21%). ¹H NMR (400 MHz, DMSO-d6): δ 7.60 (d, J=2.6 Hz, 1H), 7.37 (dd, J=8.9, 2.6 Hz, 1H), 7.10 (d, J=8.9 Hz, 1H), 6.51 (s, 2H), 5.03 (s, 2H), 1.77 (s, 3H). LCMS: m/z=276.93 [M+H]⁺.

### Example S4-6 N′-(2-(2,4-Dichlorophenoxy)acetoxy)-2-phenylacetimidamide

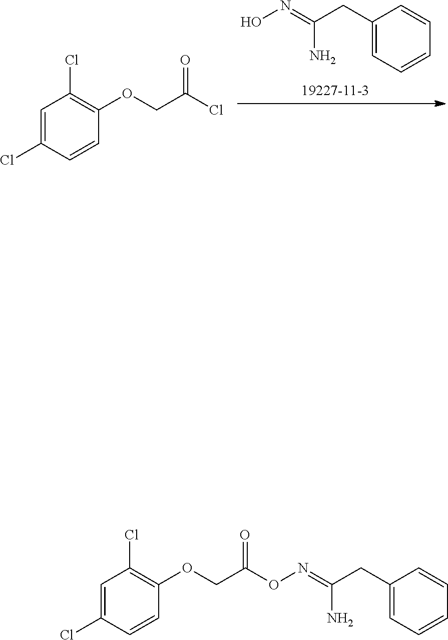 [SMILES: O=C(Cl)COc1ccc(Cl)cc1Cl ; N/C(Cc1ccccc1)=N\O ; N/C(Cc1ccccc1)=N\OC(=O)COc1ccc(Cl)cc1Cl]

To a solution of N′-hydroxy-2-phenylethanimidamide (0.94 g, 6.26 mmol) in anhydrous THF (20 mL) was added NMM (1.27 g, 12.53 mmol) and the mixture stirred for 10 min at RT. The mixture was cooled to 0° C., then 2,4-dichlorophenoxyacetyl chloride 3 (1 g, 4.17 mmol) was added under an argon atmosphere and stirred for 16 h at RT. Upon completion of the reaction, water (50 mL) was added; the mixture extracted with EtOAc (2×100 mL). The combined organic extracts were washed with brine, dried over Na₂SO₄, filtered and concentrated in vacuo which gave the crude product. Purification by flash column chromatography (silica gel; gradient elution 0-50% EtOAc:hexane) gave the title compound A17 as a white solid (0.33 g, 22%). ¹H NMR (400 MHz, DMSO-d6): δ 7.61 (d, J=2.6 Hz, 1H), 7.28 (m, 5H), 7.11 (d, J=9.0 Hz, 1H), 6.60 (s, 2H), 5.04 (s, 2H), 3.39 (s, 2H). LCMS: m/z=374.66 [M+Na]⁺.

### Example S4-7 N′-(2-(2,4-Dichlorophenoxy)acetoxy)benzimidamide

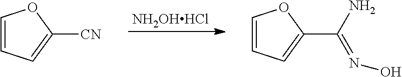

### Synthesis of Intermediate A5

To a solution of furan-2-carbonitrile (1 g, 1.07 mmol) and NH₂OH·HCl (1.5 g, 2.14 mmol) in water (10 mL) was carefully added solution of Na₂CO₃ (2.3 g, 2.14 mmol) in water (5 mL) at RT. The mixture was heated to reflux for 16 h. Upon completion of the reaction, brine (50 mL) was added; the mixture extracted with EtOAc (4×50 mL). The combined organic extracts were dried over Na₂SO₄, filtered and concentrated in vacuo which gave Intermediate A5 as a colorless liquid (1.2 g).

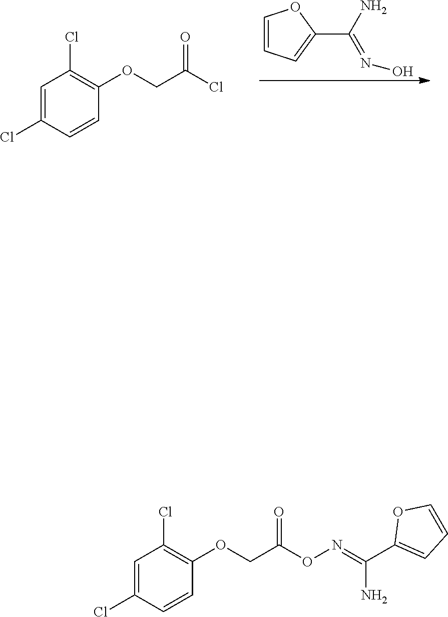 [SMILES: O=C(Cl)COc1ccc(Cl)cc1Cl ; N/C(=N\O)c1ccco1 ; N/C(=N\OC(=O)COc1ccc(Cl)cc1Cl)c1ccco1]

### Synthesis of A5

To a solution of Intermediate A5 (0.6 g, 0.47 mmol) in THF (10 mL) was added DIPEA (2.5 mL, 1.42 mmol) at 0° C. The mixture was stirred for 30 min and solution of 2,4-dichlorophenoxyacetyl chloride 3 (1.26 g, 0.52 mmol) in THF (5 mL) was added. Further the reaction was brought to RT and stirred for 15 h. Upon completion of the reaction, water (40 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-10% EtOAc:hexane) gave the title compound A5 as a brown solid (0.46 g, 30%). ¹H NMR (400 MHz, DMSO-d6): δ 7.85 (d, J=1.0 Hz, 1H), 7.62 (d, J=2.6 Hz, 1H), 7.42-7.35 (m, 1H), 7.15 (d, J=9.0 Hz, 1H), 7.09-7.02 (m, 1H), 6.98 (s, 2H), 6.66-6.60 (m, 1H), 5.13 (s, 2H). LCMS: m/z=350.72 [M+Na]⁺.

### Example S4-8 3-((2-(2,4-Dichlorophenoxy)acetoxy)imino)butan-2-one

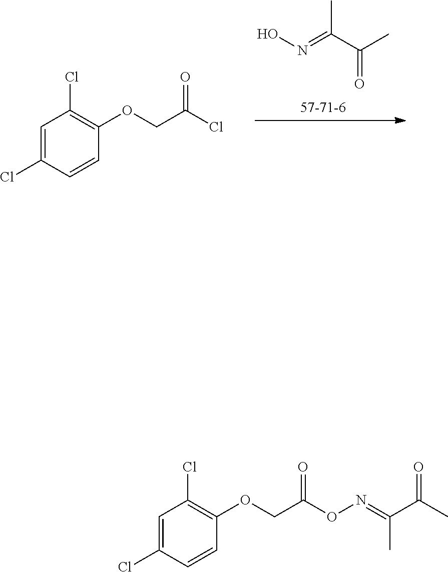

To a solution of 2,4-butanedione oxime (0.9 g, 8.39 mmol) in acetone (20 mL), potassium carbonate (3.68 g, 26.67 mmol) was added and stirred for 10 min at RT. The mixture was cooled to 0° C., then 2,4-dichlorophenoxyacetyl chloride 3 (2.13 g, 8.39 mmol) was added under an argon atmosphere and stirred for 16 h at RT. Upon completion of the reaction, water (100 mL) was added; the mixture extracted with EtOAc (2×100 mL). The combined organic extracts were washed with brine, dried over Na₂SO₄, filtered and concentrated in vacuo which gave the crude product. Purification by flash column chromatography (silica gel; gradient elution 0-50% EtOAc:hexane) gave the title compound A8 as a white solid (0.35 g, 13%). ¹H NMR (400 MHz, DMSO-d6): δ 7.64 (d, J=2.6 Hz, 1H), 7.39 (dd, J=8.9, 2.6 Hz, 1H), 7.23 (d, J=8.9 Hz, 1H), 5.31 (s, 2H), 2.41 (s, 3H), 2.00 (s, 3H). LCMS: m/z=277.86 [M+Na]⁺.

### Example S4-9 Ethyl N-2-(2,4-dichlorophenoxy)acetoxypropionimidate

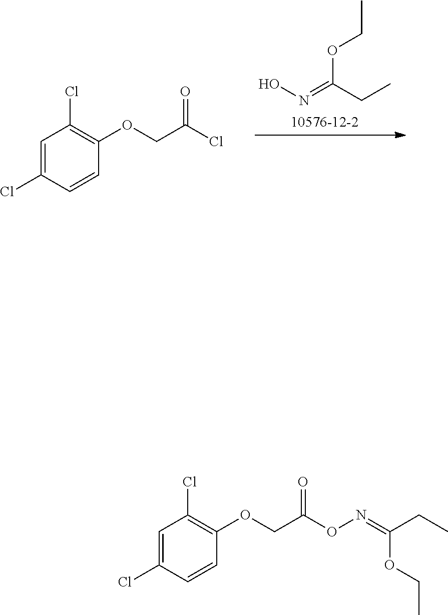

To a solution of ethyl N-hydroxyacetimidate (0.6 g, 6.26 mmol) in dry THF (20 mL), NMM (1.26 g, 12.53 mmol) was added and stirred for 10 min at RT. The mixture was cooled to 0° C., then 2,4-dichlorophenoxyacetyl chloride 3 (1 g, 4.17 mmol) was added under an argon atmosphere and stirred for 16 h at RT. Upon completion of the reaction, water (100 mL) was added; the mixture extracted with EtOAc (2×100 mL). The combined organic extracts were washed with brine, dried over Na₂SO₄, filtered and concentrated in vacuo which gave the crude product. Purification by flash column chromatography (silica gel; gradient elution 0-50% EtOAc:hexane) gave the title compound A9 as a white solid (0.8 g, 64%). ¹H NMR (400 MHz, DMSO-d6): δ 7.62 (d, J=2.6 Hz, 1H), 7.38 (dd, J=8.9, 2.6 Hz, 1H), 7.17 (d, J=8.9 Hz, 1H), 5.13 (s, 2H), 4.11 (q, J=7.0 Hz, 2H), 2.00 (s, 3H), 1.27 (t, J=7.1 Hz, 3H). LCMS: m/z=305.94 [M+H]⁺.

### Example S4-10 Methyl N-2-(2,4-dichlorophenoxy)acetoxyethanimidothioate

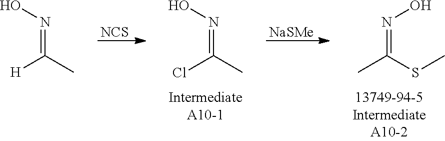

### Synthesis of Intermediate A10-1

A solution of acetaldehyde oxime (10 g, 169 mmol) in DMF (250 mL) was stirred for 10 min at RT. The mixture was cooled to 0° C., then N-chlorosuccinimide (22.6 g, 169 mmol) was added in four portions over 30 min; the resultant mixture was stirred at RT for 16 h. Upon completion of the reaction, ice water:brine mixture (200 mL: 200 mL) was added and stirred for 10 min. The mixture was extracted with EtOAc (2×150 mL); the combined organic extracts were washed with brine, dried over Na₂SO₄, filtered and concentrated in vacuo which gave Intermediate A10-1 as a white solid (0.35 g, 13%).

### Synthesis of Intermediate A10-2

To a solution of acetyl chloride oxime Intermediate A10-1 (9.5 g, 101.6 mmol) in MeOH (100 mL), a solution of sodium thiomethoxide (16.84 g, 240.5 mmol) in MeOH (150 mL) was added at 0° C. The RM was stirred for 16 h at RT. Upon completion of the reaction, EtOAc (300 mL) was added and the RM was filtered to remove solids. The filtrate was dried over Na₂SO₄, filtered and concentrated in vacuo which gave Intermediate A10-2 as a yellow solid (5.2 g).

### Synthesis of A10

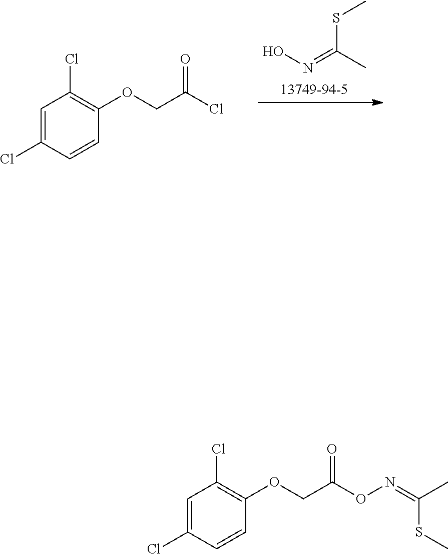

To a solution of Intermediate A10-2 (500 mg, 3.907 mmol) in CHCl₃ (20 mL) was added NMM (1.29 mL, 11.7 mmol) and the mixture stirred at RT for 10 min. The mixture was cooled to 0° C., then 2,4-dichlorophenoxyacetyl chloride 3 (1.4 g, 5.86 mmol) was added under an argon atmosphere and the mixture stirred for 16 h at RT. Upon completion of the reaction, water (100 mL) was added and the mixture extracted with EtOAc (2×100 mL). The combined organic extracts were washed with brine, dried over Na₂SO₄, filtered and concentrated in vacuo which gave the crude product. Purification by flash column chromatography (silica gel; gradient elution 0-50% EtOAc:hexane) gave the title compound A10 as a white solid (0.39 g, 12%). 1H NMR (400 MHz, CDCl₃): δ 7.39 (d, J=2.6 Hz, 1H), 7.17 (dd, J=8.8, 2.6 Hz, 1H), 6.86 (d, J=8.8 Hz, 1H), 4.84 (s, 2H), 2.39 (s, 3H), 2.29 (s, 3H). LCMS: m/z=329.73 [M+Na]⁺.

### Example S4-11 N-(2-(2,4-Dichlorophenoxy)acetoxy)benzimidoyl cyanide

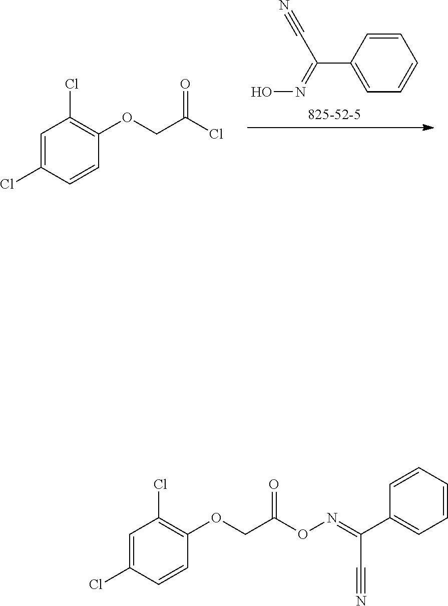 [SMILES: O=C(Cl)COc1ccc(Cl)cc1Cl ; N#C/C(=N\O)c1ccccc1 ; N#C/C(=N\OC(=O)COc1ccc(Cl)cc1Cl)c1ccccc1]

To a solution of N-hydroxybenzimidoyl cyanide (1 g, 0.68 mmol) in THF (20 mL) was added DIPEA (3.6 mL, 2.05 mmol) at 0° C. The mixture was stirred for 30 min and solution of 2,4-dichlorophenoxyacetyl chloride 3 (1.8 g, 0.75 mmol) in THF (5 mL) was added. Further the reaction was brought to RT and stirred for 15 h. Upon completion of the reaction, water (40 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-20% EtOAc:hexane) gave the title compound A6 as a pale brown solid (0.52 g, 22%). 1H NMR (400 MHz, DMSO-d6): δ 7.95-7.89 (m, 2H), 7.76-7.69 (m, 1H), 7.68-7.60 (m, 3H), 7.41-7.36 (m, 1H), 7.34-7.29 (m, 1H), 5.41 (s, 2H) LCMS: No molecular ion detected.

### Example S4-12 Diethyl 2-((2-(2,4-dichlorophenoxy)acetoxy)imino)malonate

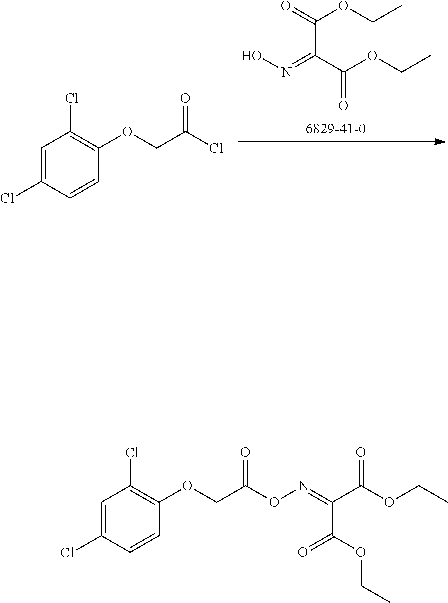 [SMILES: O=C(Cl)COc1ccc(Cl)cc1Cl ; CCOC(=O)C(=NO)C(=O)OCC ; CCOC(=O)C(=NOC(=O)COc1ccc(Cl)cc1Cl)C(=O)OCC]

To a solution of diethyl 2-(hydroxyimino)malonate (1 g, 0.52 mmol) in DCM (20 mL) was added TEA (1.2 mL, 0.89 mmol) at 0° C. The mixture was stirred for 30 min and solution of 2,4-dichlorophenoxyacetyl chloride 3 (2.2 g, 0.89 mmol) in THF (5 mL) was added. Further the reaction was brought to RT and stirred for 3 h. Upon completion of the reaction, water (40 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-20% EtOAc:hexane), followed by recrystallization from hexane:MTBE which gave the title compound A7 as a white solid (0.32 g, 16%). ¹H NMR (400 MHz, DMSO-d6): δ 7.63 (d, J=2.5 Hz, 1H), 7.38 (dd, J=8.9, 2.5 Hz, 1H), 7.23 (d, J=9.0 Hz, 1H), 5.26 (s, 2H), 4.50-4.18 (m, 4H), 1.41-1.13 (m, 6H). LCMS: No molecular ion detected.

### Example S4-13 (1,1′)-4-(-((2-(2,4-Dichlorophenoxy)acetoxy)imino) methyl)benzaldehyde-O-(2-(2,4-dichlorophenoxy)acetyl) oxime

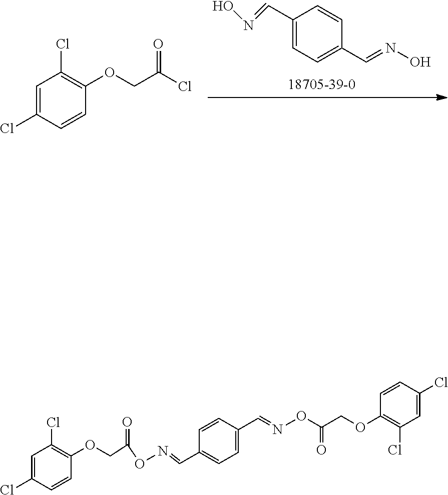 [SMILES: O=C(Cl)COc1ccc(Cl)cc1Cl ; O/N=C/c1ccc(/C=N/O)cc1 ; O=C(COc1ccc(Cl)cc1Cl)O/N=C/c1ccc(/C=N/OC(=O)COc2ccc(Cl)cc2Cl)cc1]

To a solution of 4-((hydroxyimino) methyl)benzaldehyde oxime (1 g, 0.60 mmol) in THF (40 mL) was added NMM (4.5 mL, 4.08 mmol) at 0° C. The mixture was stirred for 30 min and solution of 2,4-dichlorophenoxyacetyl chloride 3 (8.7 g, 3.65 mmol) in THF (5 mL) was added. Further the reaction was brought to RT and stirred for 15 h. Upon completion of the reaction, water (40 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-60% EtOAc:hexane), followed by trituration with MeOH which gave the title compound B3 as an off-white solid (0.75 g, 22%). ¹H NMR (400 MHz, DMSO-d6): δ 8.83 (s, 2H), 7.94 (s, 4H), 7.63 (d, J=2.4 Hz, 2H), 7.43-7.34 (m, 2H), 7.24 (d, J=8.9 Hz, 2H), 5.26 (s, 4H). LCMS: No molecular ion detected.

### Example S4-14 (2,4)-Pentane-2,4-dione O,O-di(2-(2,4-dichlorophenoxy)acetyl) dioxime

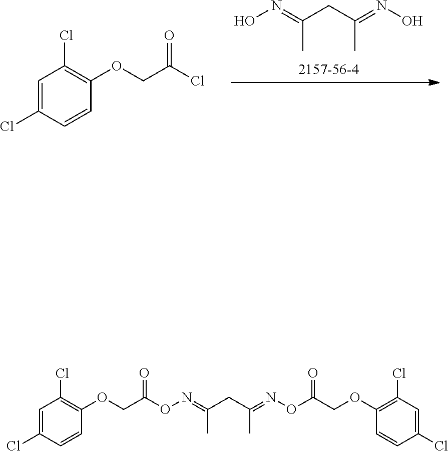

To a solution of 2,4-pentanedione dioxime (0.82 g, 6.27 mmol) in dry THF (20 mL), NMM (1.27 g, 12.53 mmol) was added and stirred for 10 min at RT. The mixture was cooled to 0° C., then 2,4-dichlorophenoxyacetyl chloride (1 g, 4.17 mmol) was added under an argon atmosphere and stirred for 16 h at RT. Upon completion of the reaction, water (100 mL) was added; the mixture extracted with EtOAc (2×100 mL). The combined organic extracts were washed with brine, dried over Na₂SO₄, filtered and concentrated in vacuo which gave the crude product. Purification by flash column chromatography (silica gel; gradient elution 0-50% EtOAc:hexane) gave the title compound B6 as a white solid (0.35 g, 11%). ¹H NMR (400 MHz, DMSO-d6): δ 7.62 (d, J=2.5 Hz, 2H), 7.37 (dd, J=8.9, 2.6 Hz, 2H), 7.15 (m, 2H), 5.15 (s, 4H), 3.46 (s, 2H), 1.98 (s, 6H). LCMS: m/z=558.32 [M+Na]⁺.

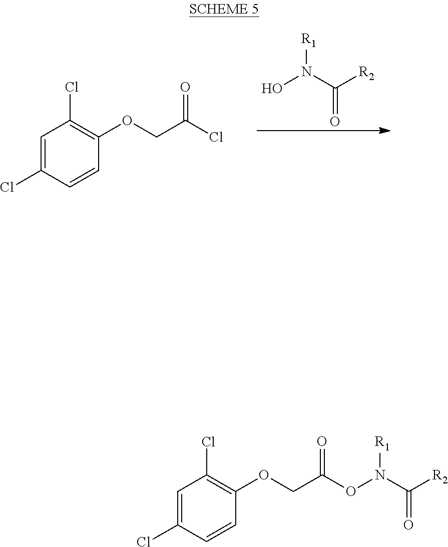

### Example S5-1 (1,4)—N°1, N°4-Bis(2-(2,4-dichlorophenoxy)acetoxy)terephthalimidamide

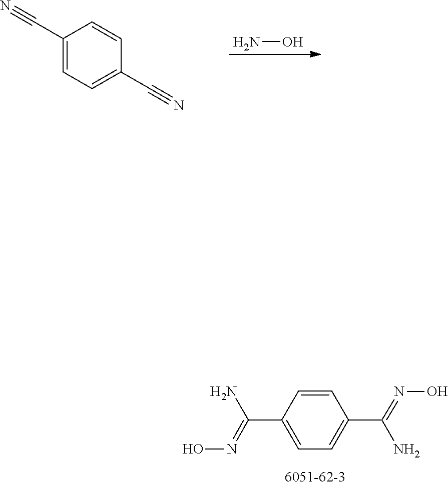

### Synthesis of Intermediate B5

To a solution of 1,4-cyanobenzene (2.5 g, 19.51 mmol) in ethanol (45 mL), Hydroxylamine in water (50% by wt.) (3.75 mL) was added and heated to reflux for 16 h at RT. Upon completion of the reaction, solid was collected by filtration which gave the Intermediate B5 as a white solid (1 g).

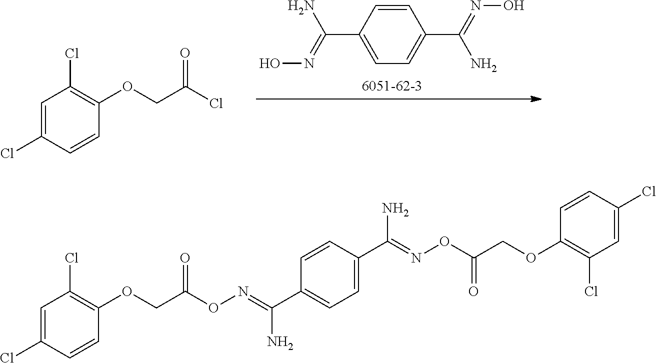 [SMILES: O=C(Cl)COc1ccc(Cl)cc1Cl ; N/C(=N\O)c1ccc(/C(N)=N/O)cc1 ; N/C(=N\OC(=O)COc1ccc(Cl)cc1Cl)c1ccc(/C(N)=N/OC(=O)COc2ccc(Cl)cc2Cl)cc1]

### Synthesis of B5

To a solution of Intermediate B5 (0.95 g, 4.89 mmol) in anhydrous THF (200 mL) was added DIPEA (25.7 mL, 48.9 mmol) and the resultant mixture stirred at RT for 10 min. The mixture was cooled to 0° C., then 2,4-dichlorophenoxyacetyl chloride 3 (6.8 g, 36.67 mmol) was added under an argon atmosphere and the reaction mixture stirred for 16 h. Upon completion of the reaction, the solid obtained was collected by filtration, which gave the title compound B5 as an off-white solid (0.5 g, 17%). ¹H NMR (400 MHz, DMSO-d6): δ 7.82 (d, J=7.1 Hz, 4H), 7.62 (d, J=2.6 Hz, 2H), 7.40 (dd, J=8.9, 2.6 Hz, 2H), 7.17 (d, J=9.0 Hz, 2H), 7.09 (s, 4H), 5.17 (s, 4H). LCMS: No molecular ion detected.

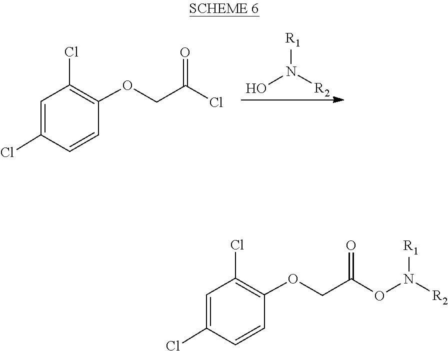

### Example S6-1 N-(2-(2,4-Dichlorophenoxy)acetoxy)benzamide

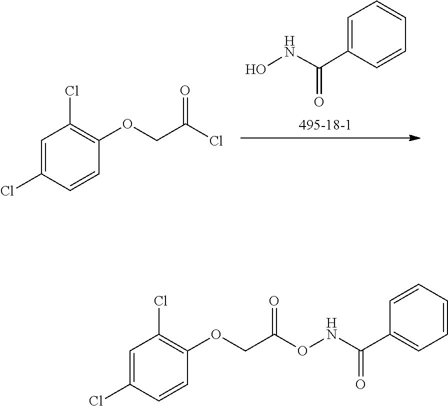 [SMILES: O=C(Cl)COc1ccc(Cl)cc1Cl ; O=C(NO)c1ccccc1 ; O=C(COc1ccc(Cl)cc1Cl)ONC(=O)c1ccccc1]

To a solution of N-hydroxybenzamide (0.62 g, 4.52 mmol), 2,4-dichlorophenoxyacetic acid (1 g, 4.52 mmol), TEA (2.2 mL g, 15.84 mmol) in DCM (15 mL) was added HATU (2.58 g, 6.78 mmol) and the reaction mixture was stirred at RT for 16 h. Upon completion of the reaction, water (40 mL) was added; the mixture extracted with DCM (2×50 mL). The combined organic extracts were washed with brine, dried over Na₂SO₄, filtered and concentrated in vacuo which gave the crude product. Purification by flash column chromatography (silica gel; gradient elution 0-20% EtOAc:hexane) gave the title compound A40 as a white solid (0.35 g, 23%). ¹H NMR (400 MHz, DMSO-d6): δ 10.16 (s, 1H), 7.57 (m, 3H), 7.28 (m, 3H), 7.05 (m, 2H), 4.86 (s, 2H). LCMS: No molecular ion detected.

### Example S6-2 N-(2-(2,4-Dichlorophenoxy)acetoxy)-N-methylbenzamide

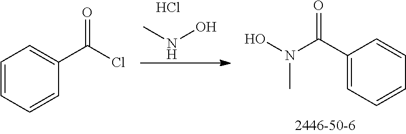

### Synthesis of Intermediate A16

To a solution of N-methylhydroxylamine hydrochloride (1.62 g, 19.4 mmol), DMAP (0.16 g, 1.29 mmol) in DCM (10 mL) triethylamine (3.92 g, 38.7 mmol) was added at RT and stirred for 10 min. The reaction mixture was cooled to 0° C., then benzoyl chloride (1.81 g, 12.9 mmol) was added dropwise and RM was stirred at RT for 16 h. Upon completion of the reaction, water (50 mL) was added; the mixture extracted with DCM (2×50 mL). The combined organic extracts were washed with brine, dried over Na₂SO₄, filtered and concentrated in vacuo which gave the crude product. Purification by flash column chromatography (silica gel; gradient elution 0-50% EtOAc:hexane) gave Intermediate A16 as a white solid (1 g).

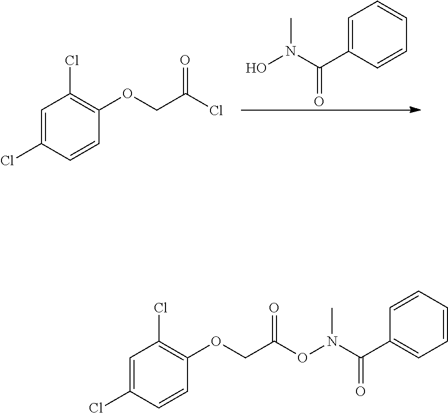 [SMILES: O=C(Cl)COc1ccc(Cl)cc1Cl ; CN(O)C(=O)c1ccccc1 ; CN(OC(=O)COc1ccc(Cl)cc1Cl)C(=O)c1ccccc1]

### Synthesis of A16

To a solution of Intermediate A16 (0.63 g, 4.17 mmol) in THF (20 mL) was added DIPEA (2.2 mL, 12.52 mmol) at 0° C. The mixture was stirred for 30 min and solution of 2,4-dichlorophenoxyacetyl chloride 3 (1.00 g, 4.17 mmol) in THF (5 mL) was added. Further the reaction was brought to RT and stirred for 3 h. Upon completion of the reaction, water (40 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-15% EtOAc:hexane) gave the title compound A16 as an off-white solid (0.45 g, 31%). ¹H NMR (400 MHz, DMSO-d6): δ 7.41 (m, 6H), 7.20 (dd, J=8.9, 2.6 Hz, 1H), 6.63 (d, J=8.9 Hz, 1H), 5.07 (s, 2H), 3.42 (s, 3H). LCMS: m/z=375.66 [M+Na]⁺.

### Example S6-3 N-(2-(2,4-dichlorophenoxy)acetoxy)-2-phenylacetamide

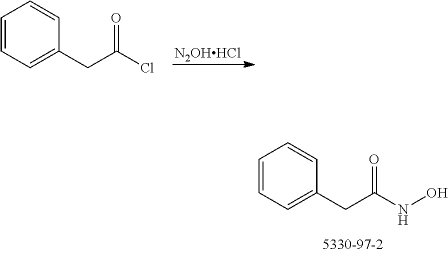

### Synthesis of Intermediate A13-1

Hydroxylamine hydrochloride (9 g, 129.37 mmol) was added to a biphasic mixture of K₂CO₃ (18 g, 129.37 mmol) in a 2:1 mixture of EtOAc (200 mL) and H₂O (100 mL). The resulting solution was cooled to 0° C., followed by dropwise addition of the phenylacetyl chloride (10 g, 64.68 mmol) dissolved in a minimum amount of EtOAc. The reaction was allowed to stir for 2 h while warming to RT. The phases were separated and the aqueous phase extracted with EtOAc (2×100 mL). The combined organic extracts were dried over Na₂SO₄, filtered, and concentrated in vacuo which gave Intermediate A13-1 as a white solid (8 g).

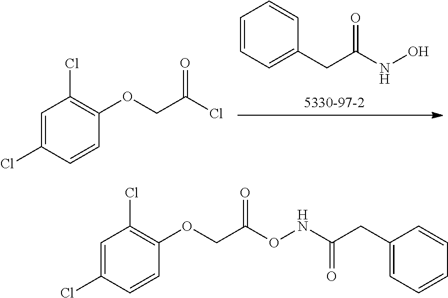 [SMILES: O=C(Cl)COc1ccc(Cl)cc1Cl ; O=C(Cc1ccccc1)NO ; O=C(Cc1ccccc1)NOC(=O)COc1ccc(Cl)cc1Cl]

### Synthesis of A13

To a solution of 2,4-dichlorophenoxyacetic acid (0.28 g, 1.26 mmol) in DCM (7 mL), DMAP (0.31 g, 2.52 mmol), Intermediate A13-1 (0.2 g, 1.32 mmol), and EDC·HCl (0.48 g, 2.52 mmol) were added and stirred for 16 h at RT. Upon completion of the reaction, water (100 mL) was added; the mixture extracted with EtOAc (2×100 mL). The combined organic extracts were washed with brine, dried over Na₂SO₄, filtered and concentrated in vacuo which gave the crude product. Purification by flash column chromatography (silica gel; gradient elution 0-25% EtOAc:hexane) gave the title compound A13 as a white solid (1 g, 43%). ¹H NMR (400 MHz, DMSO-d6): δ 12.21 (s, 1H), 7.62 (d, J=2.6 Hz, 1H), 7.38 (dd, J=8.9, 2.6 Hz, 1H), 7.21 (m, 5H), 7.16 (d, J=8.9 Hz, 1H), 5.21 (s, 2H), 3.51 (s, 2H). LCMS: m/z=375.88 [M+Na]⁺.

### Example S6-4 N¹,N⁴-bis(2-(2,6-dichlorophenoxy)acetoxy)terephthalamide

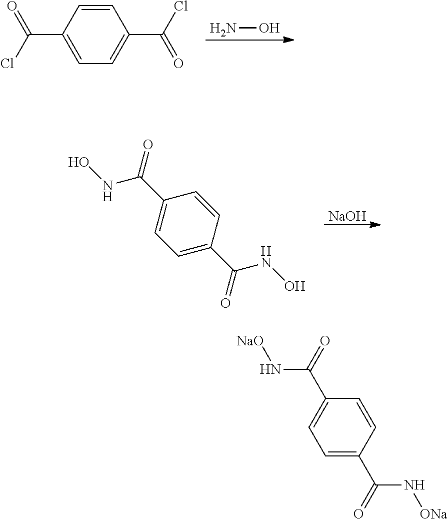

### Synthesis of Intermediate B4-1

To a solution of terephthaloyl dichloride (2.00 g, 10 mmol) in THF:H₂O (15 mL: 1.5 mL) was added NaHCO₃ (3.7 g, 44 mmol) followed by NH₂OH·HCl (1.53 g, 22 mmol) and stirred in an ice bath for 1 hr. Afterwards, the phases were separated and the aqueous phase was extracted with EtOAc (2×100 mL). The combined organic extracts were dried over Na₂SO₄, filtered, and concentrated in vacuo which gave Intermediate B4-1 as a white solid (2 g).

### Synthesis of Intermediate B4-2

To a solution of Intermediate B4-1 (2 g, 10.2 mmol) in MeOH (50 mL) was added NaOH (0.8 g, 20 mmol) and the mixture stirred at RT for 2 h. The obtained solid was filtered and dried in vacuo which gave Intermediate B4-2 as a white solid (2 g).

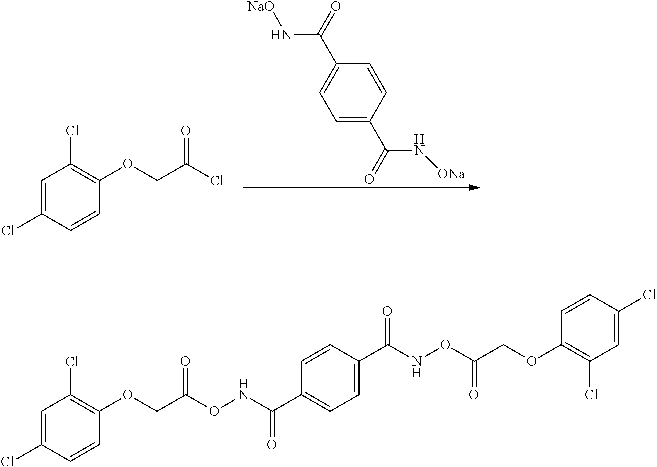

### Synthesis of B4

To a solution of Intermediate B4-2 (0.5 g, 2.08 mmol) in 1,4-dioxane (6 mL), 2,4-dichlorophenoxyacetyl chloride 3 (1.7 g, 4.58 mmol) was added and stirred for 16 h at RT. Upon completion of the reaction, water (100 mL) was added and the mixture extracted with EtOAc (2×100 mL). The combined organic extracts were washed with brine, dried over Na₂SO₄, filtered and concentrated in vacuo which gave the crude product. Purification by flash column chromatography (silica gel; gradient elution 0-50% EtOAc:hexane) gave the title compound B4 as a white solid (0.4 g, 26%). ¹H NMR (400 MHz, DMSO-d6): δ 12.85 (s, 2H), 7.96 (s, 4H), 7.64 (d, J=2.6 Hz, 2H), 7.43 (dd, J=8.9, 2.5 Hz, 2H), 7.23 (d, J=8.9 Hz, 2H), 5.33 (s, 4H). LCMS: m/z=601.80 [M+H]⁺.

### Example S6-5 Piperidin-1-yl 2-(2,4-dichlorophenoxy)acetate

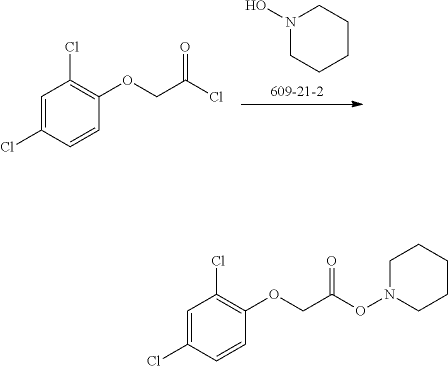 [SMILES: O=C(Cl)COc1ccc(Cl)cc1Cl ; ON1CCCCC1 ; O=C(COc1ccc(Cl)cc1Cl)ON1CCCCC1]

To a solution of N-hydroxypiperidine (1 g, 9.89 mmol) in DMF (10 mL) was added K₂CO₃ (4.1 g, 29.67 mmol) at 0° C. The mixture was stirred for 10 min and solution of 2,4-dichlorophenoxyacetyl chloride 3 (2.84 g, 11.86 mmol) in DMF (3 mL) was added. Further the reaction was brought to RT and stirred for 4 h. Upon completion of the reaction, water (40 mL) was added and the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-30% EtOAc:hexane) gave the title compound A11 as a white solid (0.3 g, 5%). ¹H NMR (400 MHz, CDCl₃): δ 7.39 (d, J=2.5 Hz, 1H), 7.16 (dd, J=8.8, 2.5 Hz, 1H), 6.82 (d, J=8.8 Hz, 1H), 4.70 (s, 2H), 3.36 (s, 2H), 2.65 (s, 2H), 1.70 (m, 4H), 1.61 (s, 1H), 1.23 (s, 1H). LCMS: m/z=303.86 [M+H]⁺.

### Example S6-6 N, N-Dibenzyl-O-(2-(2,4-dichlorophenoxy)acetyl)hydroxylamine

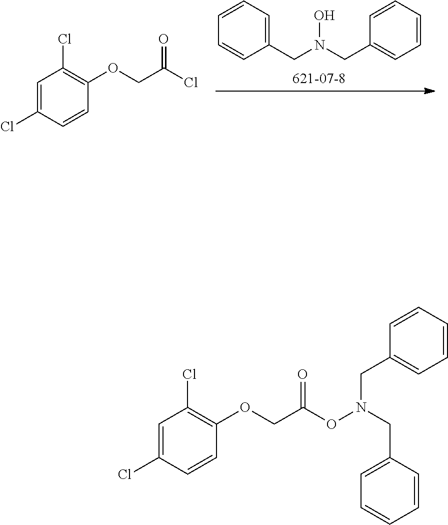 [SMILES: O=C(Cl)COc1ccc(Cl)cc1Cl ; ON(Cc1ccccc1)Cc1ccccc1 ; O=C(COc1ccc(Cl)cc1Cl)ON(Cc1ccccc1)Cc1ccccc1]

To a solution of N, N-dibenzylhydroxylamine (1 g, 4.68 mmol) in THF (20 mL) was added DIPEA (2.45 mL, 14.04 mmol) at 0° C. The mixture was stirred for 30 min and solution of 2,4-dichlorophenoxyacetyl chloride 3 (1.00 g, 5.15 mmol) in THF (5 mL) was added. Further the reaction was brought to RT and stirred for 1 h. Upon completion of the reaction, water (40 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-07% EtOAc:hexane) gave the title compound A14 as a white solid (0.7 g, 37%). ¹H NMR (400 MHz, CDCl₃): δ 7.30 (m, 10H), 7.28 (d, J=2.3 Hz, 1H), 6.83 (dd, J=8.8, 2.3 Hz, 1H), 5.68 (d, J=8.8 Hz, 1H), 4.32 (s, 2H), 4.10 (s, 4H) LCMS: No molecular ion detected.

### Example S6-7 1,3-Dioxoisoindolin-2-yl 2-(2,4-dichlorophenoxy)acetate

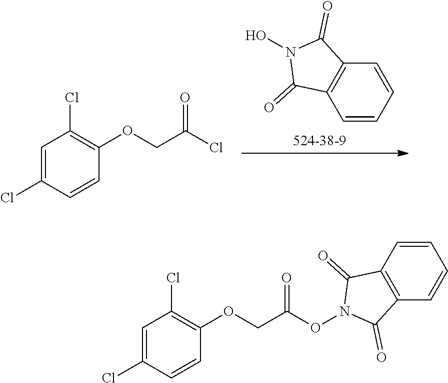 [SMILES: O=C(Cl)COc1ccc(Cl)cc1Cl ; O=C1c2ccccc2C(=O)N1O ; O=C(COc1ccc(Cl)cc1Cl)ON1C(=O)c2ccccc2C1=O]

To a solution of 2-hydroxyisoindoline-1,3-dione (1 g, 6.13 mmol) in THF (20 mL) was added DIPEA (3.2 mL, 18.39 mmol) at 0° C. The mixture was stirred for 30 min and solution of 2,4-dichlorophenoxyacetyl chloride 3 (1.62 g, 6.74 mmol) in THF (5 mL) was added. Further the reaction was brought to RT and stirred for 1 h. Upon completion of the reaction, water (40 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-07% EtOAc:hexane) gave the title compound A15 as an off-white solid (1 g, 45%). ¹H NMR (400 MHz, CDCl₃): δ 7.86 (m, 2H), 7.74 (m, 2H), 7.41 (d, J=2.5 Hz, 1H), 7.21 (m, 1H), 6.96 (d, J=8.8 Hz, 1H), 5.10 (s, 2H) LCMS: No molecular ion detected.

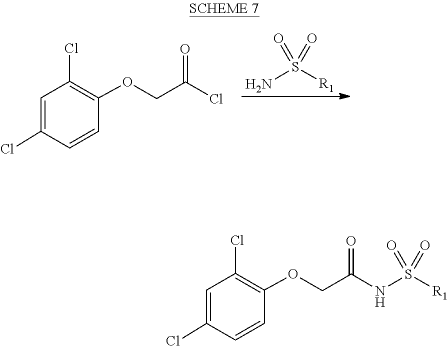

### Example 7-1 2-(2,4-Dichlorophenoxy)-N-tosylacetamide

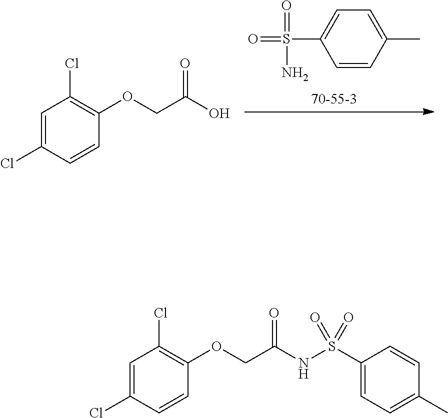 [SMILES: O=C(O)COc1ccc(Cl)cc1Cl ; Cc1ccc(S(N)(=O)=O)cc1 ; Cc1ccc(S(=O)(=O)NC(=O)COc2ccc(Cl)cc2Cl)cc1]

To a solution of 2,4-dichlorophenoxyacetic acid (5 g, 22.62 mmol) in DCM (130 mL) was added DMAP (5.54 g, 45.24 mmol), p-toluoylsulfonamide (4.07 g, 23.75 mmol) and EDC. HCl (8.67 g, 45.24 mmol) and the resultant mixture stirred at RT for 4 h. Upon completion of the reaction, water (100 mL) was added; the mixture extracted with EtOAc (2×100 mL). The combined organic extracts were washed with brine, dried over Na₂SO₄, filtered and concentrated in vacuo which gave the crude product. Purification by flash column chromatography (silica gel; gradient elution 0-25% EtOAc:hexane) gave the title compound D32 as a white solid (0.80 g, 9%). ¹H NMR (400 MHz, DMSO-d6): δ 12.44 (s, 1H), 7.81 (d, J=8.2 Hz, 2H), 7.56 (d, J=2.5 Hz, 1H), 7.43 (d, J=8.1 Hz, 2H), 7.28 (dd, J=8.9, 2.6 Hz, 1H), 6.87 (d, J=8.9 Hz, 1H), 4.79 (s, 2H), 2.40 (s, 3H). LCMS: m/z=374.09 [M+H]⁺.

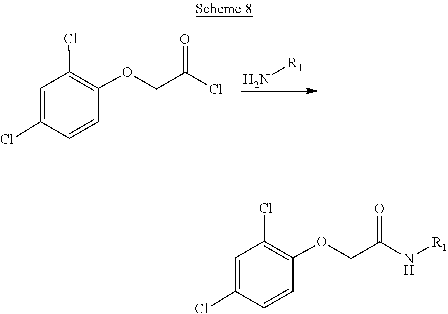

### Example S8-1 N-(4-Chlorophenyl)-2-(2,4-dichlorophenoxy)acetamide

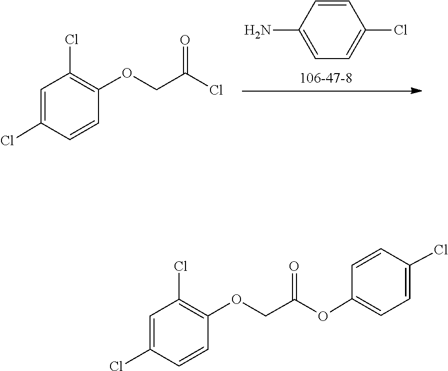 [SMILES: O=C(Cl)COc1ccc(Cl)cc1Cl ; Nc1ccc(Cl)cc1 ; O=C(COc1ccc(Cl)cc1Cl)Oc1ccc(Cl)cc1]

To a solution of 4-chloroaniline (0.5 g, 3.92 mmol) in dried DCM (20 mL) was added TEA (1.1 g, 11.75 mmol) at 0° C. The mixture was stirred for 10 min and solution of 2,4-dichlorophenoxyacetyl chloride 3 (0.93 g, 3.90 mmol) in DCM (3 mL) was added. Further the reaction was brought to RT and stirred for 16 h. Upon completion of the reaction, water (50 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-50% EtOAc:hexane) gave the title compound D6 as an off-white solid (0.37 g, 29%). ¹H NMR (400 MHz, DMSO-d6): δ 10.31 (s, 1H), 7.49 (m, 3H), 7.24 (m, 3H), 7.11 (d, J=8.9 Hz, 1H), 4.87 (s, 2H). LCMS: m/z=331.72 [M+H]⁺.

### Example S8-2 2-(2,4-Dichlorophenoxy)-N-(pyridin-4-yl)acetamide

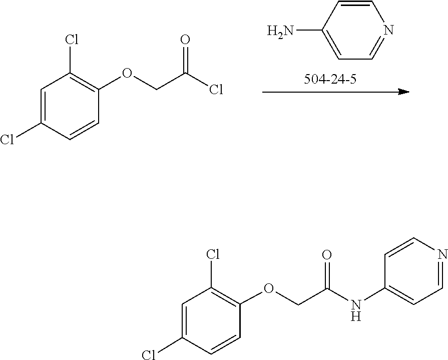 [SMILES: O=C(Cl)COc1ccc(Cl)cc1Cl ; Nc1ccncc1 ; O=C(COc1ccc(Cl)cc1Cl)Nc1ccncc1]

To a solution of 4-aminopyridine (1.0 g, 10.63 mol) in dried DCM (30 mL) was added TEA (3.23 g, 31.89 mmol) at 0° C. The mixture was stirred for 10 min and solution of 2,4-dichlorophenoxyacetyl chloride 3 (2.54 g, 10.63 mmol) in DCM (3 mL) was added. Further the reaction was brought to RT and stirred for 16 h. Upon completion of the reaction, water (50 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-50% EtOAc:hexane) gave the title compound D1 as an off-white solid (0.95 g, 30%). ¹H NMR (DMSO-d6): δ 10.58 (s, 1H), 8.45 (d, J=5.9 Hz, 2H), 7.73-7.49 (m, 3H), 7.47-7.21 (m, 1H), 7.12 (d, J=8.9 Hz, 1H), 4.92 (s, 2H). LCMS: m/z=296.84 [M+H]⁺.

### Example S8-3 2-(2,4-Dichlorophenoxy)-N-(isoxazol-3-yl)acetamide

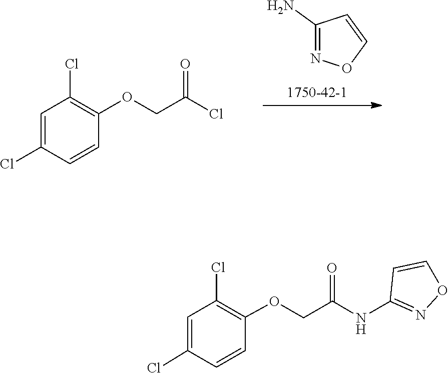 [SMILES: O=C(Cl)COc1ccc(Cl)cc1Cl ; Nc1ccon1 ; O=C(COc1ccc(Cl)cc1Cl)Nc1ccon1]

To a solution of 3-aminoisoxazole (1.0 g, 11.89 mol) in dried DCM (30 mL) was added TEA (3.6 g, 35.67 mmol) at 0° C. The mixture was stirred for 10 min and solution of 2,4-dichlorophenoxyacetyl chloride 3 (2.8 g, 11.89 mmol) in DCM (3 mL) was added. Further the reaction was brought to RT and stirred for 16 h. Upon completion of the reaction, water (50 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-50% EtOAc:hexane) gave the title compound D2 as an off-white solid (1 g, 30%). ¹H NMR (400 MHz, DMSO-d6): δ 11.33 (s, 1H), 8.83 (d, J=1.7 Hz, 1H), 7.61 (d, J=2.5 Hz, 1H), 7.37 (dd, J=8.9, 2.6 Hz, 1H), 7.09 (d, J=8.9 Hz, 1H), 6.91 (d, J=1.7 Hz, 1H), 4.95 (s, 2H). LCMS: m/z=286.78 [M+H]⁺.

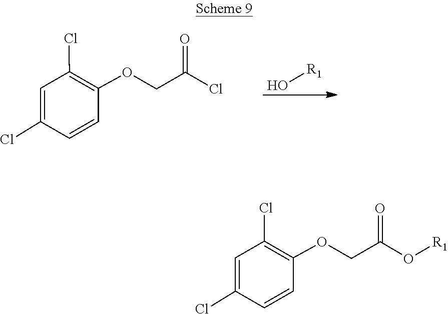

### Example S9-1 4-Chlorophenyl 2-(2,4-dichlorophenoxy)acetate

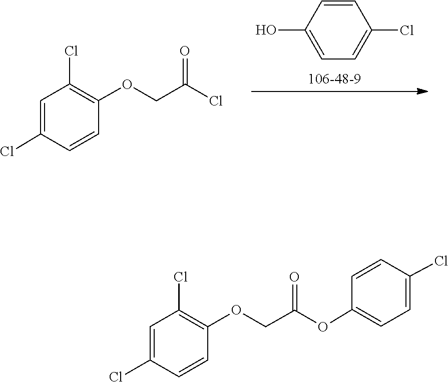 [SMILES: O=C(Cl)COc1ccc(Cl)cc1Cl ; Oc1ccc(Cl)cc1 ; O=C(COc1ccc(Cl)cc1Cl)Oc1ccc(Cl)cc1]

To a solution of 4-chlorophenol (1 g, 7.77 mmol) in THF (20 mL) was added DIPEA (4.1 mL, 23.31 mmol) at 0° C. The mixture was stirred for 30 min and solution of 2,4-dichlorophenoxyacetyl chloride 3 (2.04 g, 8.55 mmol) in THF (5 mL) was added. Further the reaction was brought to RT and stirred for 15 h. Upon completion of the reaction, water (40 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-10% EtOAc:hexane) gave the title compound E5 as a brown solid (0.36 g, 14%). ¹H NMR (400 MHz, DMSO-d6): δ 7.63 (d, J=2.5 Hz, 1H), 7.55-7.49 (m, 2H), 7.42-7.37 (m, 1H), 7.33-7.28 (m, 1H), 7.28-7.22 (m, 2H), 5.25 (s, 2H). LCMS: No molecular ion detected.

### Example S9-2 Pyrimidin-5-yl 2-(2,4-dichlorophenoxy)acetate

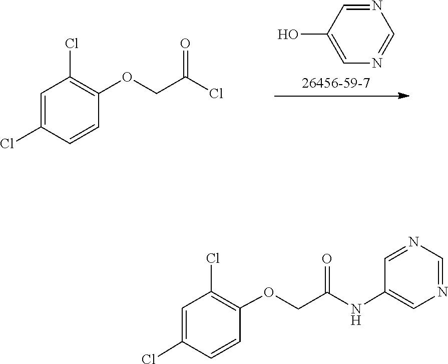 [SMILES: O=C(Cl)COc1ccc(Cl)cc1Cl ; Oc1cncnc1 ; O=C(COc1ccc(Cl)cc1Cl)Nc1cncnc1]

To a solution of pyrimidin-5-ol (0.5 g, 5.20 mmol) in THF (20 mL) was added solution of 2,4-dichlorophenoxyacetyl chloride 3 (1.25 g, 5.20 mmol) in THF (5 mL) was added. Further the reaction was stirred at RT for 3 h. Upon completion of the reaction, water (40 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-45% EtOAc:hexane) gave the title compound E14 as a white solid (0.36 g, 24%). ¹H NMR (400 MHz, DMSO-d6): δ 9.15 (s, 1H), 8.83 (s, 2H), 7.64 (d, J=2.4 Hz, 1H), 7.31 (m, 2H), 5.32 (s, 2H). LCMS: m/z=298.73 [M+H]⁺.

### Example S9-3 1,3-Dimethyl-1H-pyrazol-5-yl 2-(2,4-dichlorophenoxy)acetate

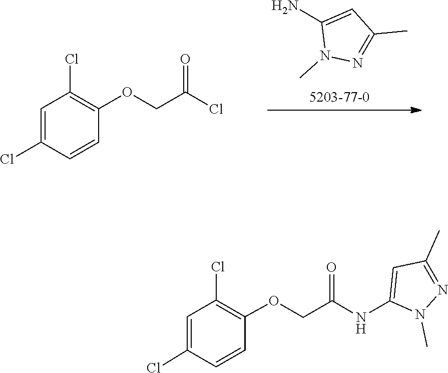

To a solution of 1,3-dimethyl-1H-pyrazol-5-ol (2 g, 17.8 mmol), 2,4-dichlorophenoxyacetyl chloride 3 (3.94 g, 17.84 mmol) and TEA (8.5 mL g, 62.44 mmol) in DMF (50 mL) was added HATU (17 g, 35.68 mmol) and the reaction mixture was stirred at RT for 3 h. Upon completion of the reaction, ice cold water (100 mL) was added; the obtained solid was filtered and dried in vacuo which gave the title compound E6 as a white solid (0.36 g, 24%). ¹H NMR (400 MHz, DMSO-d6): δ 7.64 (d, J=2.5 Hz, 1H), 7.40 (dd, J=8.9, 2.5 Hz, 1H), 7.34-7.19 (m, 1H), 5.91 (s, 1H), 5.34 (s, 2H), 3.56 (s, 3H), 2.11 (s, 3H). LCMS: No molecular ion detected.

### Example S9-4 2-(2,4-Dichlorophenoxy)-1-(2-hydroxy-1-methyl-1H-indol-3-yl)ethanone

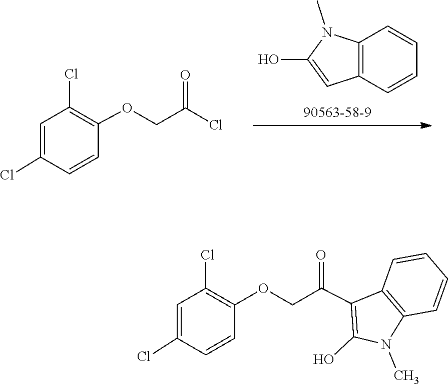

To a solution of 1-methyl-1H-indol-2-ol (0.5 g, 2.03 mmol), 2,4-dichlorophenoxyacetyl chloride 3 (0.54 g, 2.24 mmol) in dioxane (10 mL) was added KOtBu (0.7 g, 6.11 mmol). The reaction mixture was then heated at 80° C. for 6 h. Upon completion of the reaction, water (40 mL) was added and the mixture was extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-30% EtOAc:hexane) which gave the title compound C₁ as a brown solid (0.14 g, 20%). ¹H NMR (400 MHz, DMSO-d6): δ 12.31 (s, 1H), 7.74 (d, J=6.4 Hz, 1H), 7.62 (d, J=2.6 Hz, 1H), 7.36 (m, 1H), 7.30 (d, J=8.9 Hz, 1H), 7.16 (m, 1H), 6.92 (m, 2H), 5.52 (s, 2H), 3.20 (s, 3H). LCMS: No molecular ion detected.

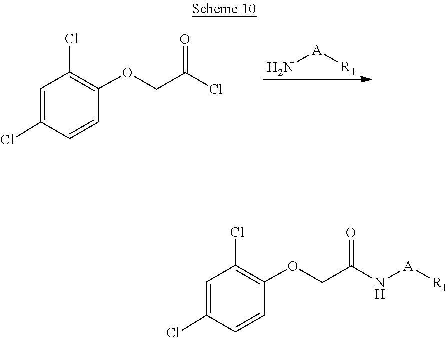

### Example S10-1 2-(2,4-Dichlorophenoxy)-N-phenoxyacetamide

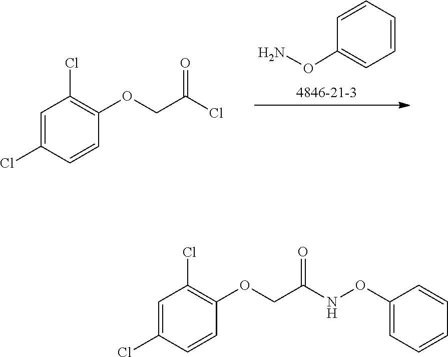 [SMILES: O=C(Cl)COc1ccc(Cl)cc1Cl ; NOc1ccccc1 ; O=C(COc1ccc(Cl)cc1Cl)NOc1ccccc1]

To a solution of O-phenylhydroxylamine (0.5 g, 4.17 mmol) in DCM (10 mL) was added TEA (0.9 mL, 6.25 mmol) at 0° C. The mixture was stirred for 30 min and solution of 2,4-dichlorophenoxyacetyl chloride 3 (1 g, 4.17 mmol) in THF (5 mL) was added. Further the reaction was brought to RT and stirred for 15 h. Upon completion of the reaction, water (40 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-30% EtOAc:hexane) gave the title compound D7 as a pale yellow solid (0.75 g, 52%). ¹H NMR (400 MHz, DMSO-d6): δ 12.21 (s, 1H), 7.62 (d, J=2.0 Hz, 1H), 7.43 (d, J=7.7 Hz, 1H), 7.34 (t, J=7.6 Hz, 2H), 7.18 (d, J=8.6 Hz, 1H), 7.04 (d, J=6.5 Hz, 3H), 4.84 (s, 2H) LCMS: m/z=333.98 [M+Na]⁺.

### Example S10-2 N-(Benzyloxy)-2-(2,4-dichlorophenoxy)acetamide

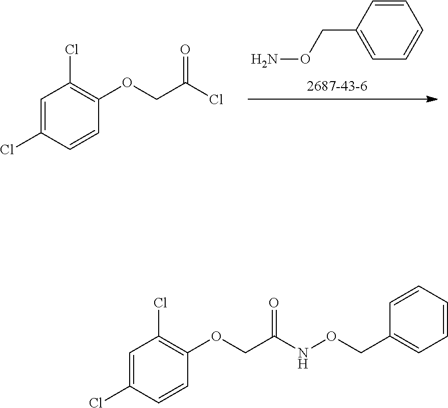 [SMILES: O=C(Cl)COc1ccc(Cl)cc1Cl ; NOCc1ccccc1 ; O=C(COc1ccc(Cl)cc1Cl)NOCc1ccccc1]

To a solution of O-benzylhydroxylamine (0.5 g, 4.17 mmol) in DCM (10 mL) was added TEA (0.9 mL, 6.25 mmol) at 0° C. The mixture was stirred for 30 min and solution of 2,4-dichlorophenoxyacetyl chloride 3 (1 g, 4.17 mmol) in DCM (5 mL) was added. The reaction mixture was allowed to warm to RT and stirred for 15 h. Upon completion of the reaction, water (40 mL) was added and the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-30% EtOAc:hexane) gave the title compound D8 as a white solid (0.5 g, 37%). ¹H NMR (400 MHz, DMSO-d6): δ 11.41 (s, 1H), 7.60 (s, 1H), 7.39 (d, J=6.3 Hz, 6H), 7.04 (d, J=8.9 Hz, 1H), 4.83 (s, 2H), 4.60 (s, 2H). LCMS: m/z=347.83 [M+Na]⁺.

### Example S10-3 2-(2,4-Dichlorophenoxy)-N′-methyl-N′-phenylacetohydrazide

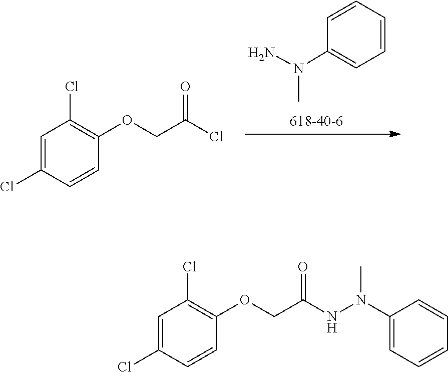

To a solution of 1-methyl-1-phenylhydrazine (0.5 g, 4.17 mmol) in DCM (10 mL) was added TEA (1.75 mL, 12.5 mmol) at 0° C. The mixture was stirred for 30 min and solution of 2,4-dichlorophenoxyacetyl chloride 3 (1 g, 4.17 mmol) in DCM (5 mL) was then added. The reaction mixture was allowed to warm to RT and stirred for 15 h. Upon completion of the reaction, water (40 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-25% EtOAc:hexane) gave the title compound D11 as a brown solid (0.3 g, 37%). ¹H NMR (400 MHz, DMSO-d6): δ 10.21 (s, 1H), 7.62 (d, J=2.6 Hz, 1H), 7.43 (dd, J=8.9, 2.6 Hz, 1H), 7.24-7.11 (m, 3H), 6.83-6.73 (m, 3H), 4.79 (s, 2H), 3.09 (s, 3H). LCMS: m/z=324.76 [M+H]⁺.

### Example S10-4 N′-(2-(2,4-Dichlorophenoxy)acetyl)-N-methylbenzohydrazide

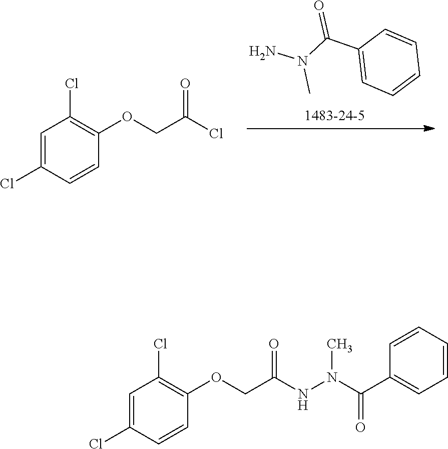 [SMILES: O=C(Cl)COc1ccc(Cl)cc1Cl ; CN(N)C(=O)c1ccccc1 ; CN(NC(=O)COc1ccc(Cl)cc1Cl)C(=O)c1ccccc1]

To a solution of N-methylbenzohydrazide (0.69 g, 4.17 mmol) in DCM (10 mL) was added TEA (0.9 mL, 6.25 mmol) at 0° C. The mixture was stirred for 30 min and solution of 2,4-dichlorophenoxyacetyl chloride 3 (1 g, 4.17 mmol) in DCM (5 mL) was added. The reaction mixture was allowed to warm to RT and stirred for 15 h. Upon completion of the reaction, water (40 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-30% EtOAc:hexane) gave the title compound D9 as a white solid (0.3 g, 20%). ¹H NMR (400 MHz, DMSO-d6): δ 10.79 (s, 1H), 7.55 (s, 1H), 7.52-7.41 (m, 3H), 7.38 (d, J=7.1 Hz, 2H), 7.10 (d, J=7.4 Hz, 1H), 6.38 (d, J=7.9 Hz, 1H), 4.54 (s, 2H), 3.13 (s, 3H). LCMS: m/z=352.84 [M+H]⁺.

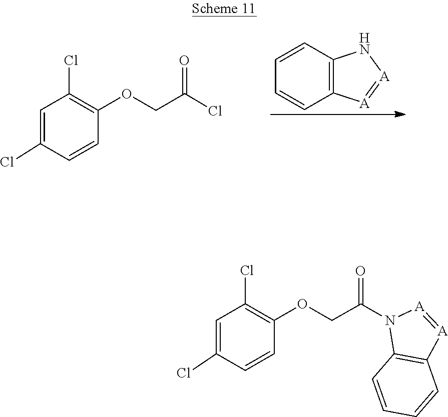

### Example S11-1 2-(2,4-Dichlorophenoxy)-1-(1H-indazol-1-yl)ethanone

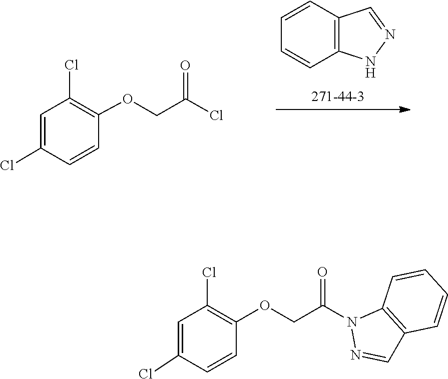 [SMILES: O=C(Cl)COc1ccc(Cl)cc1Cl ; c1ccc2[nH]ncc2c1 ; O=C(COc1ccc(Cl)cc1Cl)n1ncc2ccccc21]

To a solution of 1H-indazole (0.5 g, 4.23 mmol) in dried DCM (10 mL) was added TEA (0.85 g, 8.46 mmol) at 0° C. The mixture was stirred for 10 min and solution of 2,4-dichlorophenoxyacetyl chloride 3 (1.5 g, 6.34 mmol) in DCM (3 mL) was added. The reaction was allowed to warm to RT and stirred for 16 h. Upon completion of the reaction, water (50 mL) was added; the mixture extracted with EtOAc (2×50 mL). The combined organic extracts were washed with brine, dried over Na₂SO₄, filtered and concentrated in vacuo which gave the crude product. Purification by flash column chromatography (silica gel; gradient elution 0-50% EtOAc:hexane) gave the title compound D3 as a pale brown solid (0.31 g, 23%). ¹H NMR (400 MHz, DMSO-d6): δ 8.60 (d, J=0.8 Hz, 1H), 8.28 (dq, J=8.4, 0.9 Hz, 1H), 7.97 (dt, J=8.0, 1.0 Hz, 1H), 7.60 (m, 2H), 7.48 (ddd, J=8.0, 7.1, 0.9 Hz, 1H), 7.23 (m, 2H), 5.81 (s, 2H). LCMS: m/z=320.73 [M+H]⁺.

### Example S11-2 1-(1H-Benzo[d]imidazol-1-yl)-2-(2,4-dichlorophenoxy)ethanone

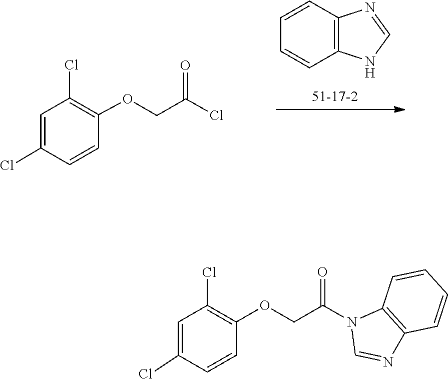 [SMILES: O=C(Cl)COc1ccc(Cl)cc1Cl ; c1ccc2[nH]cnc2c1 ; O=C(COc1ccc(Cl)cc1Cl)n1cnc2ccccc21]

To a solution of 1H-benzo[d]imidazole (2.67 g, 22.62 mmol), 2,4-dichlorophenoxy)acetic acid (5 g, 22.62 mmol) and TEA (7.7 mL g, 56.56 mmol) in DMF (50 mL) was added HATU (12.9 g, 33.93 mmol). The reaction mixture was stirred at RT for 16 h. Upon completion of the reaction, water (40 mL) was added; the precipitated solid was collected by filtration under suction and pulled dry which gave the crude product. Purification by trituration with hexane:MTBE gave the title compound D4 as an off-white solid (2.5 g, 34%). ¹H NMR (400 MHz, DMSO-d6): δ 8.94 (s, 1H), 8.08 (m, 1H), 7.75 (m, 1H), 7.65 (d, J=2.3 Hz, 1H), 7.28 (m, 4H), 5.80 (s, 2H). LCMS: No molecular ion detected.

### Evaluation of Pesticidal Activity

As previously discussed, the compounds disclosed herein exhibit varying pesticidal activity. For example, the pesticidal activity may include herbicidal activity. Exemplified below in Table 3 is select herbicidal activity for some compounds on various weeds and crops.

The following Table 2 lists the growth stages of the plants at the time of application for the results exemplified in Table 3:

TABLE 2
Plant Growth Stages at Time of Application

| | | | Plant Height |
|---|---|---|---|
| Species | Bayer Code | Leaf Stage | (cm) |
| Soybeans | GLXMA | 1^{st} Trifoliate | 8 to 12 |
| Common Ragweed | AMBEL | 4 to 6 | 5 to 7 |
| Common Lambsquarters | CHEAL | 4 to 8 | 7 to 17 |
| Velvetleaf | ABUTH | 4 to 6 | 4 to 8 |
| Tall Waterhemp | AMATA | 6 to 7 | 7 to 11 |
| Horseweed | ERICA | 4 to 6 rosette | 3 to 5 (diameter) |

The following examples applied were technical grades, including control 2,4-D Isooctyl ester (CAS 25168-26-7), except for the 2,4-D choline, which was applied as the commercially available 2,4-D choline from Corteva Agriscience LLC under the tradename ENLIST ONER. The technical grade materials were dissolved first in a 3 ml solvent mixture of 97.3% acetone:dimethyl sulfoxide (DMSO) and then diluted with a 17 ml solvent mixture of acetone, water, isopropyl alcohol, DMSO, Agri-Dex® crop oil concentrate (a heavy range, paraffin-based petroleum oil and nonionic emulsifiers commercially available from Rosecare) and nonionic Tergitol 1557 in a 40.3:45.3:11.9:1.3:1.0:0.2 v/v ratio. All mix sizes were 20 ml.

### Postemergence Application Methods for Herbicide Evaluations

Plants were grown in a soil media composed of 90% by volume Promix BX (Premier Tech Horticulture, Quakertown, PA) and 10% Profile Greens Grade (Profile Products LLC, Buffalo Grove, IL). Promix BX contains approximately 83% Sphagnum Peat Moss, 13% Perlite, 5% vermiculite, and proprietary amounts of limestone, starter fertilizer, and wetting agent. Profile Greens Grade is a sand-sized granule formed from calcining illite and montmorillonite clay. Several seeds of each species were planted in 10 cm square pots and top watered twice daily. Plant material was propagated in a warm greenhouse with temperature ranging from 25 to 28° C. and 50 to 60% relative humidity at the Corteva Global Headquarters (Indianapolis, Indiana, USA). Natural light was supplemented with 1000-watt metal halide overhead lamps with an average illumination of 500 μE m⁻² s⁻¹ photosynthetic active radiation for 16 consecutive hours each day. Plants were top-watered prior to herbicide application and sub-watered after herbicide application.

Appropriate amounts of test materials were weighed out into glass vials in their technical form to deliver either 280 or 560 g acid equivalent (ae)/ha. The samples were dissolved in 3 ml of Solvent 1. Solvent 1 is 97% acetone and 3% dimethyl sulfoxide and serves as a general-purpose solvent (GPS) for dissolving technical materials. If samples did not readily dissolve, the sample bottles were placed in hot water and sonicated for 20 minutes. This typically resulted in the test material being thoroughly dissolved. The samples were then diluted to their final concentration using 17 ml of solvent 2 for a total of 20 ml of spray solution. Solvent 2 contains acetone, water, isopropyl alcohol, DMSO, Agri-Dex® crop oil concentrate (a heavy range, paraffin-based petroleum oil and nonionic emulsifiers commercially available from Rosecare) and nonionic Tergitol 1557 in a 40.3:45.3:11.9:1.3:1.0:0.2 v/v ratio. All percentages are listed on a volume basis. 2,4-D choline as applied from the commercially available 2,4-D choline from Corteva Agriscience LLC under the tradename ENLIST ONER and diluted with solvent 2 to compare all materials in the same spray solution.

Herbicide applications were made to 2 replicates of each plant species at the 4 to 8 leaf stage, depending on the species. Treatments were applied with a research track-sprayer (Generation III Research Sprayer manufactured by DeVries Manufacturing in Hollandale, MN, USA) calibrated to deliver a spray volume of 187 L/ha. The track-sprayer was fitted with an 8003E nozzle from Spray Systems Company (North Avenue and Schmale Road, P.O. Box 7900, Wheaton, IL 60187), and used a spray pressure of 276 kPa pressure and a speed of 3.1 km/h. The nozzle height was 46 cm above the plant canopy. The experimental design used for the trials was a randomized complete block with 2 replications per treatment. After application, plants were placed on carts according to the randomization and returned to the greenhouse. Plants were sub-watered after treatment with Indianapolis city water or fertilized three times a week with a commercial fertilizer solution (Jack's Professional 15-5-15 4 Ca 2 Mg fertilizer (manufactured by JR PETERS INC., 6656 Grant Way, Allentown, PA 18106; www.jrpeters.com). Percent visual control assessments were made on a scale of 0 to 100% (where 0 was no control and 100 was complete plant death) at 7, 14, and 21 days after treatment.

Measuring the Effect of Representative Experimental Compounds on the Growth of Amaranthus rudis.

In conformity with the methods outlined above in Postemergence Application Methods for Herbicide Evaluations, the effect of representative experimental compounds on the growth of Amaranthus rudis (AMATA), common name tall water hemp, was determined at 2 different levels (respectively, 280 and 560 g acid equivalent (ae)/ha) of the experimental actives tested. Plants were treated with one of the compounds, a control plant was not treated. Twenty-one days after treatment, the plants were measured. The data is present as a percent reduction in plant size relative to the untreated control. Referring now to Table 3, some of the compounds tested demonstrated a significant effect on the growth of this common species of weed.

Measuring the Effect of Representative Experimental Compounds on the Growth of Ambrosia artemisiifolia

In conformity with the methods outlined above in Postemergence Application Methods for Herbicide Evaluations, the effect of representative experimental compounds on the growth of Ambrosia artemisiifolia (AMBEL), (common ragweed), was determined at 2 different levels (respectively, 280 and 560 g acid equivalent (ae)/ha) of the experimental actives tested. Plants were treated with one of the compounds, a control plant was not treated. Twenty-one days after treatment, the plants were measured. The data is present as a percent reduction in plant size relative to the untreated control. Referring now to Table 4, some of the compounds tested demonstrated a significant effect on the growth of this common species of weed.

Measuring the Effect of Representative Experimental Compounds on the Growth of Abutilon theophrasti

In conformity with the methods outlined above in Postemergence Application Methods for Herbicide Evaluations, the effect of representative experimental compounds on the growth of Abutilon theophrasti (ABUTH), common name velvetleaf, was determined at 2 different levels (respectively, 280 and 560 g acid equivalent (ae)/ha) of the experimental actives tested. Plants were treated with one of the compounds, a control plant was not treated. Twenty-one days after treatment, the plants were measured. The data is present as a percent reduction in plant size relative to the untreated control. Referring now to Table 3, some of the compounds tested demonstrated a significant effect on the growth of this common species of weed.

Measuring the Effect of Representative Experimental Compounds on the Growth of Chenopodium album

In conformity with the methods outlined above in Postemergence Application Methods for Herbicide Evaluations, the effect of representative experimental compounds on the growth of Chenopodium album (CHEAL), common name, common lambsquarters, was determined at 2 different levels (respectively, 280 and 560 g acid equivalent (ae)/ha) of the experimental actives tested. Plants were treated with one of the compounds, a control plant was not treated. Twenty-one days after treatment, the plants were measured. The data is present as a percent reduction in plant size relative to the untreated control. Referring now to Table 3, some of the compounds tested demonstrated a significant effect on the growth of this common species of weed.

Measuring the Effect of Representative Experimental Compounds on the Growth of Conyza canadensis (L.)

In conformity with the methods outlined above in Postemergence Application Methods for Herbicide Evaluations, the effect of representative experimental compounds on the growth of Conyza canadensis (L.), (ERICA), common name, Horseweed, was determined at 2 different levels (respectively, 280 and 560 g acid equivalent (ae)/ha) of the experimental actives tested. Plants were treated with one of the compounds, a control plant was not treated. Twenty-one days after treatment, the plants were measured. The data is present as a percent reduction in plant size relative to the untreated control. Referring now to Table 3, some of the compounds tested demonstrated a significant effect on the growth of this common species of weed.

### Measuring the Effect of Representative Experimental Compounds on the Growth of Glycine max

In conformity with the methods outlined above in Postemergence Application Methods for Herbicide Evaluations, the effect of representative experimental compounds on the growth of Glycine max (Enlist E3® traited soybeans commercially available from Corteva Agriscience®) was determined at 2 different levels (respectively, 280 and 560 g acid equivalent (ae)/ha) of the experimental actives tested. Plants were treated with one of the compounds, a control plant was not treated. Twenty-one days after treatment, the plants were measured. The data is present as a percent reduction in plant size relative to the untreated control.

TABLE 3
Average Effect of Representative Compounds on the Growth of Exemplary Plants

| | | | | | | | | Overall | |
|---|---|---|---|---|---|---|---|---|---|
| | Rate | | | | | | | Ave % | |
| Compd. | g | | AVE. | Ave. | AVE. | Ave. | Ave. | Weed | AVE. |
| No. | ae/ha | DAA | ABUTH | AMATA | AMBEL | CHEAL | ERICA | Control | GLXMA |
| B10 | 280 | 21 | 25 | 67.5 | 90 | 88.5 | 90 | 72.2 | 0 |
| B10 | 560 | 21 | 27.5 | 55 | 75 | 85 | 97.5 | 68 | 0 |
| B10 | 560 | 14 | 32.5 | 80 | 67.5 | 90 | 79.5 | 69.9 | 0 |
| B10 | 280 | 14 | 25 | 77.5 | 67.5 | 75 | 77.5 | 64.5 | 0 |
| B10 | 280 | 7 | 27.5 | 70 | 62.5 | 77.5 | 72.5 | 62 | 1 |
| B10 | 560 | 7 | 22.5 | 62.5 | 57.5 | 72.5 | 75 | 58 | 0 |
| B9 | 280 | 21 | 98.5 | 70 | 100 | 97.5 | 96.5 | 92.5 | 1 |
| B9 | 560 | 21 | 75 | 82.5 | 100 | 92.5 | 99.5 | 89.9 | 0 |
| B9 | 560 | 14 | 70 | 90 | 85 | 95 | 93 | 86.6 | 0 |
| B9 | 280 | 14 | 77.5 | 91 | 70 | 94 | 100 | 86.5 | 1.5 |
| B9 | 280 | 7 | 57.5 | 72.5 | 77.5 | 82.5 | 85 | 75 | 1.5 |
| B9 | 560 | 7 | 57.5 | 77.5 | 74 | 77.5 | 75 | 72.3 | 1 |
| B13 | 280 | 21 | 67.5 | 65 | 100 | 98.5 | 99 | 86 | 5 |
| B13 | 560 | 21 | 62.5 | 67.5 | 99 | 95 | 98.5 | 84.5 | 5 |
| B13 | 280 | 14 | 62.5 | 95 | 72.5 | 96.5 | 91.5 | 83.6 | 2.5 |
| B13 | 560 | 14 | 57.5 | 87.5 | 75 | 98 | 95 | 82.6 | 3.5 |
| B13 | 560 | 7 | 45 | 75 | 88.5 | 82.5 | 87.5 | 75.7 | 7.5 |
| B13 | 280 | 7 | 55 | 67.5 | 72.5 | 80 | 85 | 72 | 2.5 |
| B14 | 560 | 21 | 97.5 | 92.5 | 100 | 99 | 99 | 97.6 | 2.5 |
| B14 | 280 | 21 | 92 | 70 | 100 | 90 | 99 | 90.2 | 2.5 |
| B14 | 560 | 14 | 87.5 | 96 | 89.5 | 98 | 100 | 94.2 | 2.5 |
| B14 | 280 | 14 | 77.5 | 90 | 75 | 94 | 100 | 87.3 | 2.5 |
| B14 | 560 | 7 | 70 | 90 | 87.5 | 77.5 | 87.5 | 82.5 | 4 |
| B14 | 280 | 7 | 60 | 72.5 | 82.5 | 75 | 85 | 75 | 5 |
| Isooctyl | 560 | 21 | 100 | 85 | 100 | 93.5 | 99.5 | 95.6 | 0 |
| Ester | | | | | | | | | |
| Isooctyl | 280 | 21 | 97.5 | 65 | 100 | 96 | 98 | 91.3 | 0 |
| Ester | | | | | | | | | |
| Isooctyl | 560 | 14 | 98.5 | 87.5 | 90 | 96.5 | 98.5 | 94.2 | 0 |
| Ester | | | | | | | | | |
| Isooctyl | 280 | 14 | 80 | 92.5 | 77.5 | 95 | 100 | 89 | 0 |
| Ester | | | | | | | | | |
| Isooctyl | 280 | 7 | 72.5 | 80 | 82.5 | 82.5 | 85 | 80.5 | 0 |
| Ester | | | | | | | | | |
| Isooctyl | 560 | 7 | 77.5 | 85 | 65 | 77.5 | 85 | 78 | 0 |
| Ester | | | | | | | | | |
| D28 | 560 | 21 | 100 | 100 | 100 | 96.5 | 98.5 | 99 | 0 |
| D28 | 280 | 21 | 100 | 81.5 | 100 | 100 | 99.5 | 96.2 | 0 |
| D28 | 280 | 14 | 96.5 | 96 | 90 | 97 | 99.5 | 95.8 | 1 |
| D28 | 560 | 14 | 97.5 | 87.5 | 100 | 96.5 | 97.5 | 95.8 | 0 |
| D28 | 560 | 7 | 72.5 | 98 | 85 | 77.5 | 82.5 | 83.1 | 0 |
| D28 | 280 | 7 | 77.5 | 80 | 75 | 80 | 82.5 | 79 | 1 |
| D27 | 560 | 21 | 100 | 100 | 100 | 97.5 | 99 | 99.3 | 0 |
| D27 | 280 | 21 | 97 | 85 | 100 | 94 | 98.5 | 94.9 | 0 |
| D27 | 560 | 14 | 98.5 | 90 | 99 | 97.5 | 100 | 97 | 0 |
| D27 | 280 | 14 | 81.5 | 92.5 | 77.5 | 85 | 100 | 87.3 | 2.5 |
| D27 | 560 | 7 | 80 | 92 | 85 | 80 | 85 | 84.4 | 0.5 |
| D27 | 280 | 7 | 75 | 80 | 85 | 82.5 | 67.5 | 78 | 0 |
| B12 | 560 | 21 | 65 | 62.5 | 100 | 90 | 97 | 82.9 | 1 |
| B12 | 280 | 21 | 55 | 62.5 | 100 | 96.5 | 96 | 82 | 0 |
| B8 | 560 | 14 | 55 | 85 | 72.5 | 95 | 98 | 81.1 | 0 |
| B8 | 280 | 14 | 45 | 92.5 | 70 | 94 | 92.5 | 78.8 | 0 |
| B8 | 560 | 7 | 40 | 67.5 | 67.5 | 77.5 | 75 | 65.5 | 0 |
| B8 | 280 | 7 | 37.5 | 67.5 | 36 | 80 | 80 | 60.2 | 0 |
| D33 | 280 | 21 | 100 | 95 | 100 | 96.5 | 99.5 | 98.2 | 1 |
| D33 | 560 | 21 | 100 | 92.5 | 100 | 99 | 97.5 | 97.8 | 0 |
| D33 | 280 | 14 | 98.5 | 92.5 | 96 | 98 | 99.5 | 96.9 | 0 |
| D33 | 560 | 14 | 99.5 | 92.5 | 90 | 92.5 | 100 | 94.9 | 0 |
| D33 | 280 | 7 | 85 | 92.5 | 86 | 82.5 | 82.5 | 85.7 | 0 |
| D33 | 560 | 7 | 92.5 | 92 | 80 | 46.5 | 77.5 | 77.7 | 0 |
| E9 | 560 | 21 | 99.5 | 94 | 100 | 93.5 | 100 | 97.4 | 1 |
| E9 | 280 | 21 | 88.5 | 92.5 | 100 | 90 | 99 | 94 | 0.5 |
| E9 | 560 | 14 | 98 | 87.5 | 97.5 | 99 | 100 | 96.4 | 0 |
| E9 | 280 | 14 | 75 | 85 | 90 | 96.5 | 99.5 | 89.2 | 0 |
| E9 | 660 | 7 | 82.5 | 92.5 | 87.5 | 80 | 77.5 | 84 | 0 |
| E9 | 280 | 7 | 57.5 | 77.5 | 80 | 75 | 82.5 | 74.5 | 1 |
| B7 | 560 | 21 | 42.5 | 52.5 | 100 | 87.5 | 98.5 | 76.2 | 0 |
| B7 | 280 | 21 | 15 | 70 | 77.5 | 90 | 99 | 70.3 | 0 |
| B7 | 560 | 14 | 30 | 85 | 67.5 | 96.5 | 91.5 | 74.1 | 0 |
| B7 | 560 | 7 | 37.5 | 67.5 | 72.5 | 77.5 | 85 | 68 | 0 |
| B7 | 280 | 14 | 17.5 | 85 | 57.5 | 96.5 | 70 | 65.3 | 5 |
| B7 | 280 | 7 | 20 | 57.5 | 62.5 | 82.5 | 82.5 | 61 | 0 |
| D12 | 560 | 21 | 96.5 | 77.5 | 100 | 96 | 100 | 94 | 0 |
| D12 | 280 | 21 | 67.5 | 65 | 100 | 90 | 100 | 84.5 | 0 |
| D12 | 560 | 14 | 85 | 92.5 | 77.5 | 98.5 | 99.5 | 90.6 | 0 |
| D12 | 280 | 14 | 67.5 | 87.5 | 67.5 | 98.5 | 98.5 | 83.9 | 0 |
| D12 | 560 | 7 | 67.5 | 77.5 | 72.5 | 75 | 85 | 75.5 | 0 |
| D12 | 280 | 7 | 47.5 | 62.5 | 75 | 80 | 85 | 70 | 0 |
| D13 | 280 | 21 | 57.5 | 80 | 94 | 90 | 97.5 | 83.8 | 0 |
| D13 | 560 | 21 | 37.5 | 92 | 50 | 92.5 | 99 | 74.2 | 0 |
| D13 | 560 | 14 | 35 | 84 | 91 | 98 | 91.5 | 79.9 | 2.5 |
| D13 | 280 | 14 | 40 | 91 | 75 | 95 | 92.5 | 78.7 | 0 |
| D13 | 560 | 7 | 32.5 | 75 | 75 | 75 | 82.5 | 68 | 0 |
| D13 | 280 | 7 | 35 | 72.5 | 70 | 77.5 | 80 | 67 | 0 |
| D14 | 560 | 21 | 65 | 92.5 | 100 | 100 | 100 | 91.5 | 0 |
| D14 | 280 | 21 | 52.5 | 72.5 | 100 | 95 | 98.5 | 83.7 | 0 |
| D14 | 560 | 14 | 52.5 | 91.5 | 97 | 90 | 94 | 85 | 0 |
| D14 | 280 | 14 | 45 | 90 | 72.5 | 96.5 | 94 | 79.6 | 0 |
| D14 | 280 | 7 | 37.5 | 75 | 80 | 72.5 | 82.5 | 69.5 | 0 |
| D14 | 560 | 7 | 50 | 75 | 75 | 75 | 72.5 | 69.5 | 0.5 |
| A20 | 280 | 21 | 92.5 | 100 | 100 | 95 | 100 | 97.5 | 0 |
| A20 | 560 | 21 | 100 | 82.5 | 100 | 96 | 100 | 95.7 | 0 |
| A20 | 280 | 14 | 79 | 90 | 100 | 98 | 98.5 | 93.1 | 0 |
| A20 | 560 | 14 | 100 | 90 | 85 | 92.5 | 94.5 | 92.4 | 0 |
| A20 | 560 | 7 | 87.5 | 92.5 | 85 | 77.5 | 80 | 84.5 | 1.5 |
| A20 | 280 | 7 | 60 | 91.5 | 72.5 | 77.5 | 75 | 75.3 | 0 |
| A21 | 560 | 21 | 99.5 | 100 | 100 | 99 | 100 | 99.7 | 0 |
| A21 | 280 | 21 | 90 | 80 | 100 | 97.5 | 97.5 | 93 | 0 |
| A21 | 560 | 14 | 94.5 | 87.5 | 98 | 96.5 | 94 | 94.1 | 0 |
| A21 | 280 | 14 | 67.5 | 90 | 80 | 92.5 | 92 | 84.4 | 0 |
| A21 | 560 | 7 | 87.5 | 85 | 70 | 82.5 | 82.5 | 81.5 | 1 |
| A21 | 280 | 7 | 60 | 80 | 70 | 77.5 | 85 | 74.5 | 1 |
| E11 | 560 | 14 | 97.5 | 93.5 | 90 | 98 | 90 | 95.6 | 0.5 |
| E11 | 560 | 21 | 100 | 90 | 100 | 100 | 100 | 98 | 0.5 |
| E11 | 280 | 21 | 98.5 | 67.5 | 100 | 92.5 | 100 | 91.7 | 0 |
| E11 | 280 | 14 | 85 | 90 | 70 | 97.5 | 97.5 | 88 | 0 |
| E11 | 560 | 7 | 82.5 | 85 | 80 | 80 | 85 | 82.5 | 0.5 |
| E11 | 280 | 7 | 65 | 70 | 77.5 | 77.5 | 82.5 | 74.5 | 0 |
| B8 | 560 | 14 | 90 | 96.5 | 98.5 | 97 | 97.5 | 95.9 | 0 |
| B8 | 560 | 21 | 99 | 100 | 100 | 99.5 | 100 | 99.7 | 0 |
| B8 | 280 | 21 | 97 | 92.5 | 100 | 96 | 98.5 | 96.8 | 0 |
| B8 | 280 | 14 | 80 | 92.5 | 90 | 92.5 | 97 | 90.4 | 0 |
| B8 | 560 | 7 | 70 | 91.5 | 72.5 | 82.5 | 82.5 | 79.8 | 0.5 |
| B8 | 280 | 7 | 62.5 | 84 | 72.5 | 80 | 72.5 | 74.3 | 0 |
| D15 | 560 | 14 | 94.5 | 95 | 92.5 | 97.5 | 95 | 94.9 | 2.5 |
| D15 | 560 | 21 | 99.5 | 97.5 | 100 | 97.5 | 100 | 98.9 | 0 |
| D15 | 286 | 21 | 99.5 | 72.5 | 100 | 85 | 99.5 | 91.3 | 0 |
| D15 | 280 | 14 | 87.5 | 85 | 82.5 | 94 | 97 | 89.2 | 0 |
| D15 | 560 | 7 | 80 | 85 | 90 | 82.5 | 80 | 83.5 | 0 |
| D15 | 280 | 7 | 70 | 75 | 85 | 77.5 | 72.5 | 76 | 0.5 |
| D16 | 280 | 21 | 80 | 85 | 100 | 97.5 | 100 | 92.5 | 1 |
| D16 | 560 | 21 | 45 | 92.5 | 100 | 100 | 99 | 87.3 | 0 |
| D16 | 280 | 14 | 72.5 | 92.5 | 75 | 94 | 92.5 | 85.3 | 2.5 |
| D16 | 560 | 14 | 37.5 | 92.5 | 87.5 | 92.5 | 97.5 | 81.5 | 0 |
| D16 | 280 | 7 | 65 | 75 | 77.5 | 80 | 72.5 | 74 | 0 |
| D16 | 560 | 7 | 27.5 | 75 | 72.5 | 82.5 | 80 | 67.5 | 0 |
| A22 | 560 | 14 | 98.5 | 92.5 | 92.5 | 99 | 100 | 96.5 | 0 |
| A22 | 560 | 21 | 99.5 | 87.5 | 100 | 97.5 | 99.5 | 96.8 | 0 |
| A22 | 280 | 21 | 97 | 65 | 100 | 87.5 | 99 | 89.7 | 0 |
| A22 | 280 | 14 | 82.5 | 87.5 | 67.5 | 97.5 | 100 | 87 | 0 |
| A22 | 560 | 7 | 70 | 87.5 | 82.5 | 80 | 77.5 | 79.5 | 0 |
| A22 | 280 | 7 | 62.5 | 70 | 77.5 | 75 | 87.5 | 74.5 | 0 |
| D17 | 560 | 21 | 80 | 92.5 | 100 | 100 | 100 | 94.5 | 0 |
| D17 | 560 | 14 | 62.5 | 96 | 92.5 | 98 | 100 | 89.8 | 7.5 |
| D17 | 560 | 7 | 47.5 | 86.5 | 77.5 | 77.5 | 80 | 73.8 | 0 |
| D18 | 560 | 14 | 96.5 | 96 | 95 | 92.5 | 98 | 95.6 | 0 |
| D18 | 560 | 14 | 97.5 | 93.5 | 92.5 | 94 | 100 | 95.5 | 0 |
| D18 | 560 | 21 | 99.5 | 95 | 100 | 100 | 98 | 98.5 | 0 |
| D18 | 560 | 21 | 100 | 87.5 | 100 | 100 | 100 | 97.5 | 0 |
| D18 | 280 | 21 | 100 | 75 | 100 | 92.5 | 100 | 93.5 | 0 |
| D18 | 280 | 14 | 85 | 87.5 | 80 | 97.5 | 99 | 89.8 | 0 |
| D18 | 560 | 7 | 72.5 | 96 | 91 | 75 | 85 | 83.9 | 0 |
| D18 | 560 | 7 | 67.5 | 89.5 | 82.5 | 77.5 | 82.5 | 79.9 | 0 |
| D18 | 280 | 7 | 67.5 | 72.5 | 75 | 75 | 85 | 75 | 0 |
| D19 | 560 | 14 | 98 | 93.5 | 85 | 95 | 100 | 94.3 | 0 |
| D19 | 560 | 21 | 100 | 75 | 100 | 98 | 99.5 | 94.5 | 0.5 |
| D19 | 280 | 21 | 98.5 | 72.5 | 100 | 96.5 | 98.5 | 93.2 | 0 |
| D19 | 280 | 14 | 89 | 90 | 82.5 | 94 | 98 | 90.7 | 0 |
| D19 | 560 | 7 | 87.5 | 90 | 93.5 | 85 | 80 | 87.2 | 0 |
| D19 | 280 | 7 | 75 | 77.5 | 82.5 | 77.5 | 82.5 | 79 | 0 |
| D20 | 560 | 21 | 80 | 85 | 100 | 95 | 96 | 91.2 | 1 |
| D20 | 280 | 21 | 98.5 | 57.5 | 100 | 99 | 96 | 90.2 | 0.5 |
| D20 | 560 | 21 | 67.5 | 38.5 | 100 | 92.5 | 99.5 | 79.6 | 1.5 |
| D20 | 560 | 14 | 67.5 | 96 | 95 | 94.5 | 98.5 | 90.3 | 3.5 |
| D20 | 280 | 14 | 90 | 97.5 | 70 | 87.5 | 99.5 | 88.9 | 0 |
| D20 | 560 | 14 | 62.5 | 89 | 72.5 | 95 | 97.5 | 83.3 | 0 |
| D20 | 280 | 7 | 77.5 | 72.5 | 80 | 85 | 72.5 | 77.5 | 0 |
| D20 | 560 | 7 | 55 | 77.5 | 77.5 | 82.5 | 82.5 | 75 | 1 |
| D20 | 560 | 7 | 37.5 | 80 | 75 | 82.5 | 82.5 | 71.5 | 0 |
| E12 | 560 | 21 | 62.5 | 87.5 | 100 | 87.5 | 100 | 87.5 | 0.5 |
| E12 | 280 | 21 | 65 | 70 | 100 | 87.5 | 96.5 | 83.8 | 0 |
| E12 | 560 | 14 | 52.5 | 87.5 | 90 | 95 | 100 | 85 | 0 |
| E12 | 280 | 14 | 60 | 80 | 70 | 90 | 97.5 | 79.5 | 0 |
| E12 | 560 | 7 | 50 | 75 | 77.5 | 80 | 85 | 73.5 | 0 |
| E12 | 280 | 7 | 50 | 70 | 75 | 77.5 | 75 | 69.5 | 0 |
| E13 | 560 | 14 | 93.5 | 96 | 97.5 | 90 | 99 | 95.2 | 0 |
| E13 | 280 | 14 | 90 | 82.5 | 89.5 | 95 | 100 | 91.4 | 0 |
| E13 | 560 | 21 | 100 | 99 | 100 | 100 | 97.5 | 99.3 | 1 |
| E13 | 280 | 21 | 100 | 86.5 | 100 | 96.5 | 99.5 | 96.5 | 1 |
| E13 | 560 | 7 | 72.5 | 87.5 | 82.5 | 85 | 80 | 81.5 | 0 |
| E13 | 280 | 7 | 76.5 | 85 | 70 | 77.5 | 82.5 | 78.3 | 0 |
| E14 | 560 | 14 | 77.5 | 97 | 98.5 | 92.5 | 98 | 92.7 | 1.5 |
| E14 | 560 | 21 | 97.5 | 100 | 99.5 | 100 | 99 | 99.2 | 1 |
| E14 | 280 | 21 | 97.5 | 92.5 | 100 | 95 | 96 | 96.2 | 0 |
| E14 | 280 | 14 | 65 | 90 | 92.5 | 90 | 92.5 | 86 | 0 |
| E14 | 560 | 7 | 67.5 | 98.5 | 77.5 | 77.5 | 85 | 81.2 | 0 |
| E14 | 280 | 7 | 65 | 89 | 82.5 | 80 | 75 | 78.3 | 0 |
| D21 | 560 | 21 | 75 | 100 | 100 | 96 | 98 | 93.8 | 1.5 |
| D21 | 280 | 21 | 47.5 | 67.5 | 99.5 | 92.5 | 95 | 80.4 | 2.5 |
| D21 | 560 | 14 | 57.5 | 90 | 92.5 | 92.5 | 100 | 86.5 | 1 |
| D21 | 280 | 14 | 37.5 | 88 | 70 | 95 | 100 | 78.1 | 0 |
| D21 | 560 | 7 | 47.5 | 90 | 85 | 80 | 80 | 76.5 | 2.5 |
| D21 | 280 | 7 | 32.5 | 72.5 | 75 | 82.5 | 82.5 | 69 | 1 |
| D22 | 560 | 14 | 98.5 | 92.5 | 97.5 | 99 | 99 | 97.3 | 0 |
| D22 | 280 | 14 | 93.5 | 92.5 | 90 | 91.5 | 100 | 93.5 | 0 |
| D22 | 560 | 21 | 99 | 100 | 100 | 97.5 | 100 | 99.3 | 1.5 |
| D22 | 280 | 21 | 100 | 87.5 | 100 | 97.5 | 100 | 97 | 0 |
| D22 | 560 | 7 | 82.5 | 92.5 | 85 | 82.5 | 85 | 85.5 | 0 |
| D22 | 280 | 7 | 72.5 | 80 | 90 | 80 | 85 | 81.5 | 0 |
| D23 | 560 | 14 | 99 | 90 | 85 | 99 | 97.5 | 94.1 | 0 |
| D23 | 280 | 14 | 93.5 | 90 | 80 | 96.5 | 100 | 92 | 0 |
| D23 | 560 | 21 | 100 | 87.5 | 100 | 95 | 100 | 96.5 | 0 |
| D23 | 280 | 21 | 99 | 72.5 | 100 | 94 | 99 | 92.9 | 1 |
| D23 | 560 | 7 | 90 | 82.5 | 75 | 85 | 77.5 | 82 | 0 |
| D23 | 280 | 7 | 70 | 82.5 | 77.5 | 80 | 80 | 78 | 0 |
| D24 | 560 | 21 | 70 | 92.5 | 100 | 95 | 99.5 | 91.4 | 1.5 |
| D24 | 280 | 21 | 70 | 77.5 | 100 | 90 | 97.5 | 87 | 3.5 |
| D24 | 560 | 14 | 65 | 85 | 92.5 | 92.5 | 87.5 | 84.5 | 12.5 |
| D24 | 280 | 14 | 55 | 87.5 | 80 | 90 | 95 | 81.5 | 5 |
| D24 | 280 | 7 | 40 | 80 | 77.5 | 82.5 | 70 | 70 | 6.5 |
| D24 | 560 | 7 | 47.5 | 85 | 67.5 | 75 | 75 | 70 | 14 |
| A24 | 280 | 14 | 77.5 | 92.5 | 75 | 91.5 | 98.5 | 87 | 0 |
| A24 | 560 | 21 | 85 | 72.5 | 100 | 94.5 | 97 | 89.8 | 0 |
| A24 | 280 | 21 | 80 | 77.5 | 97.5 | 98.5 | 94.5 | 89.6 | 0 |
| A24 | 560 | 14 | 67.5 | 90 | 75 | 95 | 91.5 | 83.8 | 0 |
| A24 | 280 | 7 | 62.5 | 82.5 | 70 | 75 | 80 | 74 | 0 |
| A24 | 560 | 7 | 57.5 | 72.5 | 75 | 77.5 | 75 | 71.5 | 2.5 |
| A25 | 560 | 21 | 30 | 64 | 82.5 | 75 | 99 | 70.1 | 1 |
| A25 | 280 | 21 | 17.5 | 50 | 87.5 | 75 | 67.5 | 59.5 | 1 |
| A25 | 560 | 14 | 27.5 | 82.5 | 67.5 | 92.5 | 77.5 | 69.5 | 0 |
| A25 | 280 | 14 | 22.5 | 80 | 62.5 | 65 | 83.5 | 62.7 | 2.5 |
| A25 | 560 | 7 | 30 | 57.5 | 80 | 72.5 | 72.5 | 62.5 | 0 |
| A25 | 280 | 7 | 27.5 | 65 | 70 | 72.5 | 60 | 59 | 0 |
| A26 | 560 | 14 | 92.5 | 90 | 90 | 95 | 99.5 | 93.4 | 0 |
| A26 | 280 | 14 | 72.5 | 85 | 72.5 | 97.5 | 99 | 85.3 | 0 |
| A26 | 560 | 21 | 100 | 92.5 | 100 | 95 | 100 | 97.5 | 1 |
| A26 | 280 | 21 | 96 | 67.5 | 100 | 87.5 | 100 | 90.2 | 0.5 |
| A26 | 560 | 7 | 67.5 | 94 | 77.5 | 80 | 82.5 | 80.3 | 0 |
| A26 | 280 | 7 | 62.5 | 65 | 80 | 72.5 | 80 | 72 | 0 |
| A27 | 560 | 14 | 98.5 | 96 | 82.5 | 94.5 | 100 | 94.3 | 0 |
| A27 | 280 | 14 | 75 | 90 | 75 | 98 | 100 | 87.6 | 0 |
| A27 | 560 | 7 | 82.5 | 85 | 90 | 85 | 82.5 | 85 | 0 |
| A27 | 560 | 21 | 100 | 77.5 | 100 | 100 | 97.5 | 95 | 1.5 |
| A27 | 280 | 21 | 92.5 | 62.5 | 100 | 98.5 | 98.5 | 90.4 | 1.5 |
| A27 | 280 | 7 | 60 | 75 | 80 | 77.5 | 85 | 75.5 | 0 |
| D25 | 560 | 21 | 2.5 | 2.5 | 7.5 | 15 | 12.5 | 8 | 2.5 |
| D25 | 280 | 21 | 0 | 5 | 10 | 12.5 | 10 | 7.5 | 0 |
| D25 | 560 | 7 | 3.5 | 7.5 | 40 | 45 | 10 | 21.2 | 3 |
| D25 | 280 | 7 | 0 | 5 | 35 | 35 | 7.5 | 16.5 | 2.5 |
| D25 | 560 | 14 | 0 | 25 | 7.5 | 7.5 | 12.5 | 10.5 | 2.5 |
| D25 | 280 | 14 | 0 | 15 | 15 | 0 | 20 | 10 | 2.5 |
| D26 | 560 | 14 | 65 | 87.5 | 85 | 77.5 | 100 | 83 | 0 |
| D26 | 560 | 21 | 70 | 87.5 | 100 | 95 | 72.5 | 85 | 0 |
| D26 | 280 | 21 | 62.5 | 75 | 100 | 92.5 | 62.5 | 78.5 | 0 |
| D26 | 280 | 14 | 52.5 | 88.5 | 70 | 85 | 91.5 | 77.5 | 1.5 |
| D26 | 560 | 7 | 55 | 85 | 90 | 77.5 | 60 | 73.5 | 0 |
| D26 | 280 | 7 | 42.5 | 72.5 | 75 | 75 | 57.5 | 64.5 | 0 |
| A29 | 560 | 14 | 82.5 | 97 | 100 | 95 | 99 | 94.7 | 5 |
| A29 | 280 | 14 | 67.5 | 90 | 75 | 96.5 | 100 | 85.8 | 2.5 |
| A29 | 560 | 7 | 70 | 91 | 77.5 | 77.5 | 82.5 | 79.7 | 1.5 |
| A29 | 560 | 21 | 97.5 | 100 | 100 | 97.5 | 96.5 | 98.3 | 4 |
| A29 | 280 | 21 | 77.5 | 72.5 | 100 | 91.5 | 99 | 88.1 | 0 |
| A29 | 280 | 7 | 50 | 70 | 77.5 | 82.5 | 77.5 | 71.5 | 0.5 |
| A30 | 560 | 14 | 96.5 | 93.5 | 97.5 | 90 | 100 | 95.5 | 0 |
| A30 | 280 | 14 | 77.5 | 96 | 90 | 94 | 100 | 91.5 | 0 |
| A30 | 560 | 7 | 80 | 94 | 96.5 | 80 | 80 | 86.1 | 1 |
| A30 | 280 | 7 | 62.5 | 75 | 85 | 80 | 87.5 | 78 | 0 |
| A30 | 560 | 21 | 99.5 | 99 | 100 | 100 | 94.5 | 98.6 | 1 |
| A30 | 280 | 21 | 87.5 | 92.5 | 100 | 98 | 99 | 95.4 | 1.5 |
| A31 | 560 | 14 | 57.5 | 96 | 75 | 97 | 89.5 | 83 | 6.5 |
| A31 | 560 | 21 | 70 | 67.5 | 100 | 96 | 98.5 | 86.4 | 3.5 |
| A31 | 560 | 7 | 40 | 75 | 62.5 | 82.5 | 82.5 | 68.5 | 5 |
| A32 | 560 | 14 | 88.5 | 87.5 | 92.5 | 95 | 99.5 | 92.6 | 1 |
| A32 | 280 | 14 | 74 | 96 | 72.5 | 97.5 | 97 | 87.4 | 0 |
| A32 | 560 | 7 | 67.5 | 90 | 75 | 75 | 75 | 76.5 | 0 |
| A32 | 280 | 7 | 55 | 70 | 75 | 82.5 | 87.5 | 74 | 0 |
| A32 | 560 | 21 | 98.5 | 94 | 100 | 97.5 | 98 | 97.6 | 0 |
| A32 | 280 | 21 | 87.5 | 60 | 100 | 97.5 | 99 | 88.8 | 1 |
| D1 | 280 | 7 | 78 | 68 | 60 | 68 | 70 | 69 | 0 |
| D1 | 560 | 7 | 78 | 78 | 65 | 75 | 65 | 72 | 1 |
| D1 | 280 | 14 | 88 | 75 | 89 | 78 | 88 | 83 | 0 |
| D1 | 560 | 14 | 80 | 85 | 94 | 85 | 90 | 87 | 0 |
| D1 | 280 | 21 | 93 | 78 | 78 | 97 | 99 | 89 | 0 |
| D1 | 560 | 21 | 87 | 90 | 99 | 93 | 97 | 93 | 0 |
| D2 | 280 | 7 | 78 | 70 | 68 | 70 | 68 | 71 | 0 |
| D2 | 560 | 7 | 88 | 78 | 73 | 73 | 73 | 77 | 3 |
| D2 | 280 | 14 | 83 | 85 | 89 | 73 | 88 | 83 | 0 |
| D2 | 560 | 14 | 93 | 92 | 97 | 88 | 94 | 92 | 3 |
| D2 | 280 | 21 | 80 | 94 | 100 | 78 | 91 | 88 | 0 |
| D2 | 560 | 21 | 99 | 97 | 97 | 97 | 99 | 98 | 3 |
| E1 | 280 | 7 | 93 | 78 | 65 | 70 | 68 | 75 | 0 |
| E1 | 560 | 7 | 85 | 73 | 73 | 75 | 75 | 76 | 1 |
| E1 | 280 | 14 | 96 | 75 | 96 | 73 | 85 | 85 | 0 |
| E1 | 560 | 14 | 97 | 85 | 90 | 90 | 85 | 89 | 1 |
| E1 | 280 | 21 | 100 | 70 | 99 | 78 | 95 | 88 | 1 |
| E1 | 560 | 21 | 100 | 85 | 97 | 87 | 98 | 93 | 1 |
| E2 | 280 | 7 | 85 | 78 | 63 | 65 | 70 | 72 | 0 |
| E2 | 560 | 7 | 85 | 73 | 63 | 75 | 73 | 74 | 0 |
| E2 | 280 | 14 | 93 | 80 | 90 | 63 | 88 | 83 | 0 |
| E2 | 560 | 14 | 93 | 83 | 95 | 88 | 85 | 89 | 0 |
| E2 | 280 | 21 | 98 | 83 | 94 | 63 | 94 | 86 | 1 |
| E2 | 560 | 21 | 98 | 84 | 99 | 94 | 90 | 93 | 0 |
| B1 | 280 | 7 | 5 | 40 | 23 | 15 | 13 | 19 | 0 |
| B1 | 560 | 7 | 13 | 40 | 20 | 55 | 28 | 31 | 0 |
| B1 | 280 | 14 | 8 | 25 | 18 | 25 | 30 | 21 | 0 |
| B1 | 560 | 14 | 5 | 35 | 18 | 53 | 35 | 29 | 0 |
| B1 | 280 | 21 | 8 | 30 | 0 | 20 | 8 | 13 | 0 |
| B1 | 560 | 21 | 3 | 48 | 5 | 33 | 15 | 21 | 0 |
| E3 | 280 | 7 | 75 | 78 | 63 | 68 | 73 | 71 | 0 |
| E3 | 560 | 7 | 83 | 78 | 75 | 70 | 78 | 77 | 0 |
| E3 | 280 | 14 | 78 | 78 | 89 | 78 | 83 | 81 | 0 |
| E3 | 560 | 14 | 93 | 85 | 93 | 73 | 88 | 86 | 0 |
| E3 | 280 | 21 | 86 | 75 | 95 | 80 | 95 | 86 | 3 |
| E3 | 560 | 21 | 97 | 83 | 98 | 77 | 93 | 89 | 0 |
| B2 | 280 | 7 | 80 | 68 | 75 | 65 | 75 | 73 | 0 |
| B2 | 560 | 7 | 85 | 70 | 75 | 65 | 78 | 75 | 0 |
| B2 | 280 | 14 | 93 | 70 | 88 | 78 | 88 | 83 | 0 |
| B2 | 560 | 14 | 93 | 80 | 97 | 85 | 90 | 89 | 0 |
| B2 | 280 | 21 | 98 | 75 | 88 | 84 | 94 | 88 | 0 |
| B2 | 560 | 21 | 99 | 80 | 100 | 97 | 97 | 94 | 0 |
| A1 | 280 | 7 | 83 | 73 | 65 | 70 | 73 | 73 | 0 |
| A1 | 560 | 7 | 85 | 78 | 80 | 70 | 73 | 77 | 0 |
| A1 | 280 | 14 | 96 | 85 | 88 | 78 | 90 | 87 | 0 |
| A1 | 560 | 14 | 97 | 87 | 97 | 89 | 90 | 92 | 0 |
| A1 | 280 | 21 | 97 | 80 | 95 | 83 | 93 | 89 | 0 |
| A1 | 560 | 21 | 100 | 82 | 100 | 98 | 98 | 95 | 0 |
| A2 | 280 | 7 | 85 | 68 | 65 | 70 | 68 | 71 | 0 |
| A2 | 560 | 7 | 85 | 75 | 73 | 73 | 73 | 76 | 0 |
| A2 | 280 | 14 | 94 | 70 | 92 | 70 | 93 | 84 | 0 |
| A2 | 560 | 14 | 96 | 88 | 96 | 85 | 85 | 90 | 0 |
| A2 | 280 | 21 | 98 | 73 | 91 | 73 | 90 | 87 | 1 |
| A2 | 560 | 21 | 96 | 87 | 100 | 87 | 98 | 93 | 0 |
| A3 | 280 | 7 | 53 | 75 | 63 | 75 | 68 | 67 | 10 |
| A3 | 560 | 7 | 68 | 78 | 75 | 68 | 75 | 73 | 2 |
| A3 | 280 | 14 | 55 | 75 | 78 | 83 | 85 | 75 | 8 |
| A3 | 560 | 14 | 68 | 90 | 95 | 83 | 90 | 85 | 0 |
| A3 | 280 | 21 | 58 | 83 | 90 | 85 | 90 | 81 | 4 |
| A3 | 560 | 21 | 72 | 70 | 90 | 87 | 95 | 83 | 0 |
| D3 | 280 | 7 | 30 | 70 | 63 | 68 | 63 | 59 | 0 |
| D3 | 560 | 7 | 48 | 73 | 68 | 68 | 68 | 65 | 0 |
| D3 | 280 | 14 | 25 | 73 | 75 | 63 | 83 | 64 | 0 |
| D3 | 560 | 14 | 43 | 80 | 90 | 70 | 85 | 74 | 0 |
| D3 | 280 | 21 | 23 | 75 | 73 | 63 | 84 | 63 | 0 |
| D3 | 560 | 21 | 35 | 78 | 95 | 70 | 88 | 73 | 0 |
| A4 | 280 | 7 | 35 | 75 | 60 | 60 | 60 | 58 | 0 |
| A4 | 560 | 7 | 50 | 60 | 68 | 68 | 65 | 62 | 0 |
| A4 | 280 | 14 | 40 | 75 | 78 | 63 | 83 | 68 | 0 |
| A4 | 560 | 14 | 58 | 73 | 88 | 75 | 85 | 76 | 0 |
| A4 | 280 | 21 | 38 | 70 | 88 | 63 | 88 | 69 | 0 |
| A4 | 560 | 21 | 53 | 75 | 85 | 83 | 88 | 77 | 3 |
| A5 | 280 | 7 | 38 | 70 | 60 | 60 | 70 | 60 | 0 |
| A5 | 560 | 7 | 43 | 65 | 60 | 63 | 73 | 61 | 0 |
| A5 | 280 | 14 | 40 | 73 | 78 | 68 | 83 | 68 | 0 |
| A5 | 560 | 14 | 53 | 68 | 85 | 63 | 85 | 71 | 0 |
| A5 | 280 | 21 | 30 | 73 | 78 | 68 | 88 | 67 | 0 |
| A5 | 560 | 21 | 50 | 70 | 85 | 65 | 88 | 72 | 0 |
| A6 | 280 | 7 | 78 | 80 | 68 | 68 | 65 | 72 | 3 |
| A6 | 560 | 7 | 90 | 75 | 70 | 68 | 73 | 75 | 10 |
| A6 | 280 | 14 | 75 | 83 | 93 | 83 | 88 | 84 | 2 |
| A6 | 560 | 14 | 85 | 78 | 95 | 78 | 88 | 84 | 3 |
| A6 | 280 | 21 | 80 | 73 | 98 | 94 | 98 | 88 | 4 |
| A6 | 560 | 21 | 94 | 81 | 100 | 73 | 98 | 89 | 4 |
| A7 | 280 | 7 | 75 | 73 | 63 | 68 | 68 | 69 | 0 |
| A7 | 560 | 7 | 88 | 78 | 70 | 70 | 73 | 76 | 2 |
| A7 | 280 | 14 | 83 | 83 | 83 | 75 | 89 | 82 | 0 |
| A7 | 560 | 14 | 89 | 88 | 95 | 73 | 83 | 85 | 1 |
| A7 | 280 | 21 | 92 | 90 | 92 | 73 | 93 | 88 | 1 |
| A7 | 560 | 21 | 94 | 88 | 98 | 78 | 90 | 89 | 1 |
| B3 | 280 | 7 | 33 | 60 | 23 | 65 | 55 | 47 | 0 |
| B3 | 560 | 7 | 60 | 70 | 48 | 68 | 65 | 62 | 0 |
| B3 | 280 | 14 | 13 | 58 | 45 | 68 | 78 | 52 | 0 |
| B3 | 560 | 14 | 58 | 68 | 50 | 70 | 80 | 65 | 0 |
| B3 | 280 | 21 | 12 | 58 | 45 | 68 | 85 | 53 | 0 |
| B3 | 560 | 21 | 58 | 68 | 53 | 70 | 80 | 66 | 0 |
| D4 | 280 | 7 | 85 | 75 | 60 | 68 | 70 | 72 | 0 |
| D4 | 560 | 7 | 93 | 83 | 68 | 65 | 70 | 76 | 0 |
| D4 | 280 | 14 | 95 | 75 | 85 | 75 | 83 | 83 | 0 |
| D4 | 560 | 14 | 96 | 97 | 95 | 85 | 94 | 93 | 0 |
| D4 | 280 | 21 | 98 | 75 | 85 | 83 | 88 | 86 | 0 |
| D4 | 560 | 21 | 98 | 95 | 98 | 93 | 99 | 97 | 1 |
| A8 | 280 | 7 | 74 | 70 | 70 | 68 | 70 | 70 | 0 |
| A8 | 560 | 7 | 95 | 75 | 80 | 70 | 73 | 79 | 0 |
| A8 | 280 | 14 | 65 | 83 | 90 | 73 | 85 | 79 | 0 |
| A8 | 560 | 14 | 96 | 85 | 98 | 80 | 90 | 90 | 1 |
| A8 | 280 | 21 | 70 | 78 | 96 | 83 | 92 | 84 | 0 |
| A8 | 560 | 21 | 99 | 87 | 100 | 80 | 97 | 93 | 0 |
| A9 | 280 | 7 | 80 | 65 | 73 | 70 | 70 | 72 | 0 |
| A9 | 560 | 7 | 80 | 78 | 83 | 65 | 70 | 75 | 0 |
| A9 | 280 | 14 | 80 | 63 | 93 | 83 | 91 | 82 | 0 |
| A9 | 560 | 14 | 96 | 85 | 93 | 68 | 84 | 85 | 0 |
| A9 | 280 | 21 | 89 | 60 | 92 | 88 | 99 | 85 | 0 |
| A9 | 560 | 21 | 95 | 84 | 96 | 75 | 97 | 89 | 0 |
| E4 | 280 | 7 | 90 | 68 | 68 | 73 | 70 | 74 | 0 |
| E4 | 560 | 7 | 90 | 78 | 73 | 70 | 75 | 77 | 0 |
| E4 | 280 | 14 | 97 | 65 | 89 | 83 | 88 | 84 | 0 |
| E4 | 560 | 14 | 97 | 93 | 97 | 73 | 93 | 90 | 0 |
| E4 | 280 | 21 | 99 | 70 | 99 | 88 | 95 | 90 | 0 |
| E4 | 560 | 21 | 98 | 91 | 99 | 75 | 99 | 92 | 1 |
| A10 | 280 | 7 | 83 | 80 | 70 | 65 | 63 | 72 | 0 |
| A10 | 560 | 7 | 88 | 78 | 75 | 70 | 68 | 76 | 1 |
| A10 | 280 | 14 | 90 | 80 | 85 | 75 | 85 | 83 | 0 |
| A10 | 560 | 14 | 92 | 73 | 92 | 83 | 83 | 84 | 0 |
| A10 | 280 | 21 | 98 | 78 | 100 | 83 | 93 | 90 | 1 |
| A10 | 560 | 21 | 94 | 83 | 96 | 53 | 94 | 84 | 0 |
| A11 | 280 | 7 | 83 | 73 | 65 | 68 | 65 | 71 | 0 |
| A11 | 560 | 7 | 85 | 75 | 78 | 70 | 68 | 75 | 0 |
| A11 | 280 | 14 | 93 | 89 | 90 | 75 | 90 | 87 | 0 |
| A11 | 560 | 14 | 89 | 85 | 97 | 80 | 85 | 87 | 0 |
| A11 | 280 | 21 | 99 | 85 | 95 | 80 | 95 | 91 | 0 |
| A11 | 560 | 21 | 95 | 88 | 100 | 97 | 95 | 95 | 5 |
| A12 | 280 | 7 | 85 | 75 | 65 | 68 | 65 | 72 | 0 |
| A12 | 560 | 7 | 93 | 78 | 75 | 68 | 68 | 76 | 0 |
| A12 | 280 | 14 | 90 | 97 | 83 | 80 | 90 | 88 | 0 |
| A12 | 560 | 14 | 97 | 83 | 96 | 78 | 91 | 89 | 0 |
| A12 | 280 | 21 | 96 | 86 | 97 | 86 | 99 | 93 | 0 |
| A12 | 560 | 21 | 99 | 88 | 99 | 80 | 97 | 92 | 0 |
| A13 | 280 | 7 | 68 | 75 | 65 | 68 | 60 | 67 | 0 |
| A13 | 560 | 7 | 80 | 70 | 70 | 65 | 65 | 70 | 3 |
| A13 | 280 | 14 | 70 | 80 | 85 | 68 | 78 | 76 | 0 |
| A13 | 560 | 14 | 85 | 74 | 94 | 78 | 97 | 85 | 0 |
| A13 | 280 | 21 | 75 | 91 | 90 | 73 | 83 | 82 | 1 |
| A13 | 560 | 21 | 93 | 70 | 97 | 91 | 99 | 90 | 1 |
| B4 | 280 | 7 | 70 | 68 | 58 | 63 | 68 | 65 | 0 |
| B4 | 560 | 7 | 80 | 70 | 65 | 63 | 68 | 69 | 0 |
| B4 | 280 | 14 | 78 | 68 | 88 | 70 | 85 | 78 | 0 |
| B4 | 560 | 14 | 73 | 78 | 84 | 68 | 95 | 79 | 0 |
| B4 | 280 | 21 | 83 | 65 | 95 | 75 | 99 | 83 | 0 |
| B4 | 560 | 21 | 84 | 68 | 87 | 65 | 100 | 81 | 3 |
| A14 | 280 | 7 | 38 | 63 | 50 | 60 | 43 | 51 | 0 |
| A14 | 560 | 7 | 28 | 55 | 40 | 58 | 40 | 44 | 0 |
| A14 | 280 | 14 | 23 | 50 | 63 | 68 | 50 | 51 | 0 |
| A14 | 560 | 14 | 20 | 48 | 50 | 63 | 50 | 46 | 0 |
| A14 | 280 | 21 | 13 | 53 | 58 | 70 | 43 | 47 | 2 |
| A14 | 560 | 21 | 15 | 43 | 33 | 63 | 40 | 39 | 1 |
| A15 | 280 | 7 | 78 | 73 | 73 | 68 | 68 | 72 | 0 |
| A15 | 560 | 7 | 85 | 73 | 75 | 70 | 65 | 74 | 0 |
| A15 | 280 | 14 | 83 | 82 | 95 | 73 | 78 | 82 | 0 |
| A15 | 560 | 14 | 95 | 78 | 88 | 70 | 88 | 84 | 0 |
| A15 | 280 | 21 | 88 | 84 | 98 | 80 | 88 | 87 | 0 |
| A15 | 560 | 21 | 99 | 83 | 95 | 75 | 95 | 89 | 0 |
| D5 | 280 | 7 | 30 | 73 | 48 | 65 | 63 | 56 | 4 |
| D5 | 560 | 7 | 40 | 73 | 65 | 73 | 53 | 61 | 9 |
| D5 | 280 | 14 | 23 | 78 | 68 | 70 | 78 | 63 | 2 |
| D5 | 560 | 14 | 35 | 75 | 90 | 73 | 75 | 70 | 5 |
| D5 | 280 | 21 | 25 | 75 | 58 | 65 | 73 | 59 | 3 |
| D5 | 560 | 21 | 25 | 73 | 85 | 68 | 68 | 63 | 5 |
| D6 | 280 | 7 | 50 | 68 | 58 | 60 | 65 | 60 | 0 |
| D6 | 560 | 7 | 60 | 63 | 58 | 73 | 60 | 63 | 0 |
| D6 | 280 | 14 | 33 | 84 | 78 | 65 | 83 | 68 | 0 |
| D6 | 560 | 14 | 58 | 70 | 68 | 75 | 78 | 70 | 0 |
| D6 | 280 | 21 | 35 | 80 | 88 | 68 | 83 | 71 | 0 |
| D6 | 560 | 21 | 53 | 65 | 63 | 68 | 75 | 65 | 0 |
| E5 | 280 | 7 | 78 | 73 | 68 | 70 | 68 | 71 | 0 |
| E5 | 560 | 7 | 85 | 78 | 75 | 68 | 70 | 75 | 0 |
| E5 | 280 | 14 | 93 | 75 | 89 | 73 | 90 | 84 | 0 |
| E5 | 560 | 14 | 93 | 89 | 93 | 83 | 83 | 88 | 0 |
| E5 | 280 | 21 | 98 | 78 | 90 | 80 | 95 | 88 | 0 |
| E5 | 560 | 21 | 97 | 86 | 98 | 94 | 90 | 93 | 0 |
| E6 | 280 | 7 | 83 | 70 | 65 | 63 | 63 | 69 | 13 |
| E6 | 560 | 7 | 83 | 75 | 73 | 70 | 65 | 73 | 15 |
| E6 | 280 | 14 | 88 | 82 | 80 | 65 | 83 | 79 | 30 |
| E6 | 560 | 14 | 93 | 80 | 88 | 85 | 83 | 86 | 13 |
| E6 | 280 | 21 | 96 | 70 | 93 | 58 | 96 | 82 | 20 |
| E6 | 560 | 21 | 94 | 77 | 96 | 90 | 95 | 90 | 8 |
| C1 | 280 | 7 | 68 | 63 | 48 | 68 | 60 | 61 | 13 |
| C1 | 560 | 7 | 0 | 0 | 0 | 0 | 0 | 0 | 8 |
| C1 | 280 | 14 | 63 | 70 | 65 | 78 | 85 | 72 | 28 |
| C1 | 560 | 14 | 0 | 0 | 0 | 0 | 0 | 0 | 3 |
| C1 | 280 | 21 | 63 | 68 | 65 | 77 | 90 | 72 | 25 |
| C1 | 560 | 21 | 0 | 0 | 0 | 0 | 0 | 0 | 2 |
| D7 | 280 | 7 | 68 | 73 | 63 | 65 | 63 | 66 | 0 |
| D7 | 560 | 7 | 80 | 80 | 73 | 68 | 63 | 73 | 10 |
| D7 | 280 | 14 | 68 | 83 | 83 | 70 | 83 | 77 | 0 |
| D7 | 560 | 14 | 85 | 90 | 89 | 75 | 85 | 85 | 23 |
| D7 | 280 | 21 | 70 | 83 | 95 | 70 | 95 | 82 | 0 |
| D7 | 560 | 21 | 92 | 83 | 97 | 78 | 95 | 89 | 13 |
| D8 | 280 | 7 | 83 | 68 | 68 | 63 | 68 | 70 | 0 |
| D8 | 560 | 7 | 83 | 75 | 75 | 63 | 70 | 73 | 0 |
| D8 | 280 | 14 | 93 | 85 | 96 | 65 | 88 | 85 | 0 |
| D8 | 560 | 14 | 91 | 85 | 95 | 83 | 85 | 88 | 0 |
| D8 | 280 | 21 | 99 | 73 | 99 | 75 | 95 | 88 | 3 |
| D8 | 560 | 21 | 96 | 83 | 99 | 97 | 92 | 93 | 0 |
| D9 | 280 | 7 | 43 | 65 | 60 | 65 | 65 | 60 | 1 |
| D9 | 560 | 7 | 43 | 68 | 73 | 65 | 68 | 63 | 3 |
| D9 | 280 | 14 | 45 | 75 | 83 | 73 | 83 | 72 | 0 |
| D9 | 560 | 14 | 48 | 70 | 94 | 78 | 88 | 75 | 2 |
| D9 | 280 | 21 | 45 | 83 | 85 | 95 | 94 | 80 | 0 |
| D9 | 560 | 21 | 50 | 73 | 99 | 90 | 65 | 75 | 2 |
| A16 | 280 | 7 | 85 | 65 | 70 | 63 | 68 | 70 | 0 |
| A16 | 560 | 7 | 88 | 83 | 78 | 68 | 75 | 78 | 2 |
| A16 | 280 | 14 | 93 | 73 | 83 | 70 | 85 | 81 | 0 |
| A16 | 560 | 14 | 96 | 83 | 97 | 80 | 92 | 89 | 1 |
| A16 | 280 | 21 | 94 | 65 | 88 | 68 | 93 | 81 | 0 |
| A16 | 560 | 21 | 99 | 87 | 100 | 95 | 100 | 96 | 1 |
| D10 | 280 | 7 | 88 | 68 | 73 | 63 | 63 | 71 | 0 |
| D10 | 560 | 7 | 83 | 80 | 83 | 65 | 65 | 75 | 4 |
| D10 | 280 | 14 | 85 | 94 | 91 | 68 | 83 | 84 | 0 |
| D10 | 560 | 14 | 96 | 96 | 97 | 83 | 88 | 92 | 1 |
| D10 | 280 | 21 | 91 | 85 | 93 | 73 | 93 | 87 | 0 |
| D10 | 560 | 21 | 100 | 85 | 100 | 85 | 99 | 94 | 2 |
| B5 | 280 | 7 | 0 | 25 | 18 | 15 | 5 | 13 | 3 |
| B5 | 560 | 7 | 3 | 25 | 15 | 13 | 25 | 16 | 0 |
| B5 | 280 | 14 | 10 | 0 | 8 | 20 | 30 | 14 | 0 |
| B5 | 560 | 14 | 3 | 18 | 15 | 15 | 35 | 17 | 0 |
| B5 | 280 | 21 | 10 | 15 | 5 | 40 | 15 | 17 | 0 |
| B5 | 560 | 21 | 0 | 33 | 18 | 13 | 28 | 18 | 1 |
| B6 | 280 | 7 | 78 | 75 | 68 | 70 | 68 | 72 | 0 |
| B6 | 560 | 7 | 83 | 73 | 70 | 70 | 75 | 74 | 0 |
| B6 | 280 | 14 | 90 | 75 | 93 | 88 | 93 | 88 | 0 |
| B6 | 560 | 14 | 86 | 80 | 93 | 78 | 88 | 85 | 0 |
| B6 | 280 | 21 | 92 | 75 | 96 | 93 | 98 | 91 | 2 |
| B6 | 560 | 21 | 89 | 83 | 100 | 75 | 99 | 89 | 0 |
| E7 | 280 | 7 | 80 | 78 | 63 | 68 | 65 | 71 | 0 |
| E7 | 560 | 7 | 85 | 80 | 78 | 75 | 70 | 78 | 1 |
| E7 | 280 | 14 | 84 | 95 | 93 | 73 | 83 | 85 | 0 |
| E7 | 560 | 14 | 89 | 85 | 97 | 93 | 88 | 90 | 1 |
| E7 | 280 | 21 | 90 | 93 | 96 | 70 | 93 | 88 | 0 |
| E7 | 560 | 21 | 92 | 89 | 100 | 70 | 95 | 89 | 0 |
| A17 | 280 | 7 | 48 | 73 | 65 | 70 | 65 | 64 | 8 |
| A17 | 560 | 7 | 68 | 70 | 80 | 68 | 65 | 70 | 18 |
| A17 | 280 | 14 | 65 | 78 | 86 | 78 | 85 | 78 | 8 |
| A17 | 560 | 14 | 80 | 80 | 96 | 85 | 83 | 85 | 30 |
| A17 | 280 | 21 | 65 | 83 | 92 | 85 | 97 | 84 | 5 |
| A17 | 560 | 21 | 83 | 89 | 99 | 90 | 94 | 91 | 25 |
| D11 | 280 | 7 | 60 | 80 | 55 | 70 | 50 | 63 | 5 |
| D11 | 560 | 7 | 63 | 78 | 63 | 70 | 70 | 69 | 18 |
| D11 | 280 | 14 | 65 | 80 | 75 | 83 | 75 | 76 | 3 |
| D11 | 560 | 14 | 75 | 83 | 78 | 85 | 88 | 82 | 22 |
| D11 | 280 | 21 | 68 | 79 | 94 | 85 | 73 | 80 | 0 |
| D11 | 560 | 21 | 82 | 92 | 90 | 93 | 90 | 89 | 23 |
| 2,4-D Ch | 280 | 7 | 68 | 73 | 68 | 68 | 68 | 69 | 0 |
| 2,4-D Ch | 560 | 7 | 83 | 75 | 75 | 70 | 73 | 75 | 0 |
| 2,4-D Ch | 280 | 14 | 70 | 70 | 88 | 68 | 85 | 76 | 0 |
| 2,4-D Ch | 560 | 14 | 95 | 90 | 89 | 83 | 88 | 89 | 0 |
| 2,4-D Ch | 280 | 21 | 73 | 73 | 85 | 73 | 96 | 80 | 0 |
| 2,4-D Ch | 560 | 21 | 99 | 88 | 99 | 85 | 97 | 93 | 0 |
| Untreated | | 7 | 0 | 0 | 0 | 0 | 0 | 0 | 0 |
| Untreated | | 14 | 0 | 0 | 0 | 0 | 0 | 0 | 0 |
| Untreated | | 21 | 0 | 0 | 0 | 0 | 0 | 0 | 0 |
| 2,4-D Ch | 560 | 14 | 91 | 96 | 87.5 | 95 | 100 | 93.9 | 0 |
| 2,4-D Ch | 280 | 14 | 77.5 | 89 | 77.5 | 95 | 99.5 | 87.7 | 1 |
| 2,4-D Ch | 560 | 7 | 72.5 | 86.5 | 85 | 82.5 | 87.5 | 82.8 | 0 |
| 2,4-D Ch | 280 | 7 | 62.5 | 72.5 | 77.5 | 80 | 82.5 | 75 | 0 |
| 2,4-D Ch | 560 | 21 | 99.5 | 85 | 90 | 100 | 99 | 94.7 | 0 |
| 2,4-D Ch | 280 | 21 | 82.5 | 92.5 | 100 | 97.5 | 98.5 | 94.2 | 0.5 |
| Untreated | | 7 | 0 | 0 | 0 | 0 | 0 | 0 | 1.5 |
| Untreated | | 14 | 0 | 0 | 0 | 0 | 0 | 0 | 1 |
| Untreated | | 21 | 0 | 0 | 0 | 0 | 0 | 0 | 1.5 |

### Volatility of Compounds Measured by Thermogravimetic Analysis (TGA)

Briefly, the rate of weight loss measured in units of μg/min can be used to determine vapor pressure as shown by the simplified equation:

$$\frac{- dm}{dt} = k \times A$$

wherein, k is the intrinsic volatility and A is the surface area,

Referring now to FIG. 2. A graph of weight loss as function of time for solid samples produces a linear curve; the slope of the curve is the rate of weight loss. When this method is used to measure the volatility of aqueous salt concentrates or liquid concentrates, the water or acetone present in the sample volatilizes before the sample of interest. The first linear loss region of the curve is the loss of water or acetone, and the second linear region is characteristic of the sample of interest.

In order to illustrate the difference in volatility between some of the compounds disclosed herein and the widely used 2,4-D, data from both 2,4-D choline and exemplary compounds of the present disclosure presented below in Table 4.

TABLE 4
Volatility Data Measured by TGA

| Compd. | TGA Data |
|---|---|
| No. | (μg/min) |
| 2,4-D Ch | 1.4 |
| A1 | 0.5 |
| A10 | 0.6 |
| A11 | 4 |
| A12 | 5.8 |
| A13 | 1.2 |
| A14 | 0.4 (70° C.) |
| A15 | 0.1 |
| A16 | 4.3 |
| A17 | 4.1 |
| A19 | 0.348 |
| A2 | 0.8 |
| A20 | 2.946* |
| A20 | 1.83 |
| A20 | 1.83* |
| A21 | 1.352* |
| A22 | 0.42 |
| A23 | 0.3728 |
| A24 | 0.03549 |
| A25 | 0.8976 |
| A26 | 1.599* |
| A27 | 1.27* |
| A28 | 0.3455 |
| A29 | 0.38 |
| A3 | 1 |
| A30 | 0.9679 |
| A32 | 0.809 |
| A32 | 0.87 |
| A4 | 1 |
| A5 | 1.2 |
| A6 | 7.2 |
| A7 | 4.7 |
| A8 | 1 |
| A9 | 0.5 |
| B1 | 0.03 |
| B10 | 0.02488 |
| B12 | 0.2274 |
| B13 | 0.2318 |
| B15 | 0.3 |
| B2 | 0.04 |
| B3 | 0.8 |
| B4 | 0.3 |
| B5 | 0.1 |
| B6 | 3.7 |
| B7 | 0.03572 |
| B8 | 0.3521 |
| B8 | 0.38 |
| B8 | 0.38 |
| B9 | 0.1482 |
| C1 | 0.7 |
| D1 | 0.05 |
| D10 | 0.3 |
| D11 | 0.06 |
| D13 | 0.06009 |
| D14 | 0.01234 |
| D15 | 6.9* |
| D16 | 0.03658 |
| D17 | 0.0287 |
| D18 | 0.1777 |
| D19 | 0.61 |
| D2 | 0.2 |
| D20 | 0.0112 |
| D21 | 0.07078 |
| D22 | 0.2649 |
| D23 | 0.6973 |
| D24 | 0.08298 |
| D26 | 0.09713 |
| D27 | 0.01 |
| D28 | 0.06 |
| D3 | 0.03 |
| D33 | 0.34 |
| D4 | 0.2 |
| D5 | 0.02 |
| D6 | 0.02 |
| D7 | 0.2 |
| D8 | 0.04 |
| D9 | 0.2 |
| E1 | 0.8 |
| E11 | 0.4827 |
| E12 | 0.04464 |
| E13 | 0.16 |
| E14 | 0.0575 |
| E15 | 0.03683 |
| E3 | 2.4 |
| E4 | 0.4 |
| E5 | 0.6 |
| E6 | 1 |
| E7 | 1.5 |
| E8 | 0.7322 |
| E9 | 0.6748 |

*Indicates decomposition of the material was observed

### Safened Compositions

A safened composition comprising compounds according to Formula I may be mixed with or applied in combination with (b) an azole carboxylate safener or an agriculturally acceptable salt or ester thereof.

In some aspects, (a) and (b) are used in an amount sufficient to induce an unexpectedly enhanced herbicidal effect (e.g., increased damage or injury to undesirable vegetation) while still showing good crop compatibility (e.g., no increased damage to crops or minimal increased damage or injury to crops) when compared to the individual application of the herbicidal compounds (a) or (b). In some aspects, the damage or injury to undesirable vegetation caused by the safened compositions and methods disclosed herein is evaluated using a scale from 0% to 100%, when compared with the untreated control vegetation, wherein 0% indicates no damage to the undesirable vegetation and 100% indicates complete destruction of the undesirable vegetation. Similarly, in some aspects, the damage or injury to the crop caused by the safened compositions and methods disclosed herein is evaluated using a scale from 0% to 100%, when compared with control crops treated with only the herbicide or the safener, wherein 0% indicates no damage to the crop and 100% indicates complete destruction of the crop.

In some aspects, the joint action of (a) the compounds according to Formula I and (b) the azole carboxylate safener or an agriculturally acceptable salt or ester thereof results in unexpectedly enhanced herbicidal effect against undesirable vegetation, even at application rates below those typically used for the herbicide to have a herbicidal effect on its own. In some aspects, the compositions and methods disclosed herein can, based on the individual components, be used at lower application rates to achieve a herbicidal effect comparable to the effect produced by the individual components at normal application rates. In some aspects, the joint action of (a) the compounds according to Formula I and (b) the azole carboxylate safener or an agriculturally acceptable salt or ester thereof results in an unexpected protective effect for desired crops against damage to the crops that might be caused by the herbicide alone.

In some aspects, the weight ratio of (a) the compounds according to Formula I (in g ae/ha) to (b) the azole carboxylate safener or an agriculturally acceptable salt or ester thereof (in g ai/ha) can be 1:5 or more, such as 1:4.75 or more, 1:4.5 or more, 1:4.25 or more, 1:4 or more, 1:3.75 or more, 1:3.5 or more, 1:3.25 or more, 1:3 or more, 1:2.75 or more, 1:2.5 or more, 1:2.25 or more, 1:2 or more, 1:1.9 or more, 1:1.8 or more, 1:1.7 or more, 1:1.6 or more, 1:1.5 or more, 1:1.4 or more, 1:1.3 or more, 1:1.2 or more, 1:1.1 or more, 1:1 or more, 1.1:1 or more, 1.2:1 or more, 1.3:1 or more, 1.4:1 or more, 1.5:1 or more, 1.6:1 or more, 1.7:1 or more, 1.8:1 or more, 1.9:1 or more, 2:1 or more, 2.25:1 or more, 2.5:1 or more, 2.75:1 or more, 3:1 or more, 3.25:1 or more, 3.5:1 or more, 3.75:1 or more, 4:1 or more, 4.25:1 or more, 4.5:1 or more, 4.75:1 or more, or 5:1 or more; the weight ratio of (a) to (b) can be 5:1 or less, such as 4.75:1 or less, 4.5:1 or less, 4.25:1 or less, 4:1 or less, 3.75:1 or less, 3.5:1 or less, 3.25:1 or less, 3:1 or less, 2.75:1 or less, 2.5:1 or less, 2.25:1 or less, 2:1 or less, 1.9:1 or less, 1.8:1 or less, 1.7:1 or less, 1.6:1 or less, 1.5:1 or less, 1.4:1 or less, 1.3:1 or less, 1.2:1 or less, 1.1:1 or less, 1:1 or less, 1:1.1 or less, 1:1.2 or less, 1:1.3 or less, 1:1.4 or less, 1:1.5 or less, 1:1.6 or less, 1:1.7 or less, 1:1.8 or less, 1:1.9 or less, 1:2 or less, 1:2.25 or less, 1:2.5 or less, 1:2.75 or less, 1:3 or less, 1:3.25 or less, 1:3.5 or less, 1:3.75 or less, 1:4 or less, 1:4.25 or less, 1:4.5 or less, or 1:4.75 or less; or the weight ratio of (a) to (b) can range from any of the minimum ratios to any of the maximum ratios provided above, such as from 1:5 to 5:1, from 1:2 to 5:1, from 1:1 to 3:1, from 1:3 to 4:1, or from 1:1.5 to 3.5:1.

In some aspects, (a) and (b), independently, can be employed in a purity of from 90% to 100% (e.g., from 95% to 100%) according to nuclear magnetic resonance (NMR) spectroscopy.

### Azole Carboxylate Safeners

In addition to the compounds according to Formula I, the compositions and methods of the present disclosure can include an azole carboxylate safener or an agriculturally acceptable salt or ester thereof. Herbicide safeners are molecules used in combination with herbicides to make them “safer”—that is, to reduce the herbicidal effect of the herbicide on crop plants and to improve selectivity between crops and the undesirable vegetation being targeted by the herbicide. Herbicide safeners can be used to pre-treat crop seeds prior to planting. Herbicide safeners may also be sprayed on plants as a mixture with the herbicide, or separately and sequentially with the herbicide.

Azoles are a class of five-membered nitrogen heterocyclic ring compounds containing at least one additional heteroatom (e.g., nitrogen, sulfur, or oxygen) within the heterocyclic ring. Examples of azoles include, for example, pyrazoles, imidazoles, thiazoles, oxazoles, isoxazoles and triazoles.

Azole carboxylate safeners are a class of safeners based on carboxylate-substituted azole moieties. Examples of azole carboxylate safeners include pyrazole carboxylate safeners, imidazole carboxylate safeners, thiazole carboxylate safeners, oxazole carboxylate safeners, isoxazole carboxylate safeners, and triazole carboxylate safeners. In some aspects, the composition can include an azole carboxylate safener selected from the group consisting of fenchlorazole, flurazole, furilazole, isoxadifen, mefenpyr, agriculturally acceptable salts or esters thereof, or combinations thereof. In some aspects, the azole carboxylate safener can include fenchlorazole-ethyl, isoxadifen-ethyl, mefenpyr-diethyl, or combinations thereof.

In some aspects, the azole carboxylate safener can comprise fenchlorazole, shown below, or an agriculturally acceptable salt or ester thereof. Fenchlorazole's safening activity is described in The Pesticide Manual, Eighteenth Edition, 2016.

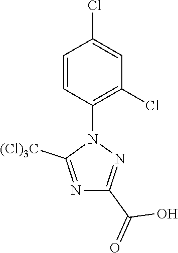

In some aspects, the fenchlorazole is provided as an agriculturally acceptable salt or ester. An exemplary agriculturally acceptable ester of fenchlorazole is fenchlorazole-ethyl, shown below.

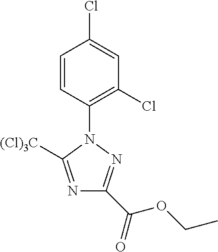

In some aspects, the azole carboxylate safener can comprise flurazole, shown below, or an agriculturally acceptable salt or ester thereof. Flurazole's safening activity is described in The Pesticide Manual, Eighteenth Edition, 2016.

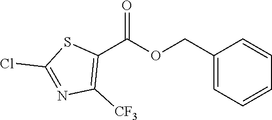 [SMILES: O=C(OCc1ccccc1)c1sc(Cl)nc1C(F)(F)F]

In some aspects, the azole carboxylate safener can comprise furilazole, shown below, or an agriculturally acceptable salt or ester thereof. Furilazole's safening activity is described in The Pesticide Manual, Eighteenth Edition, 2016.

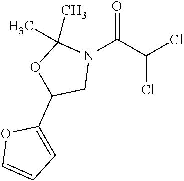 [SMILES: CC1(C)OC(c2ccco2)CN1C(=O)C(Cl)Cl]

In some aspects, the azole carboxylate safener can comprise isoxadifen, shown below, or an agriculturally acceptable salt or ester thereof. Isoxadifen's safening activity is described in The Pesticide Manual, Eighteenth Edition, 2016.

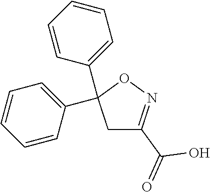 [SMILES: O=C(O)C1=NOC(c2ccccc2)(c2ccccc2)C1]

In some aspects, the isoxadifen is provided as an agriculturally acceptable salt or ester. An exemplary agriculturally acceptable ester of isoxadifen is isoxadifen-ethyl, shown below.

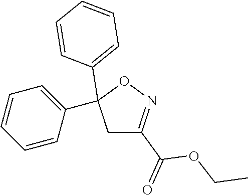 [SMILES: CCOC(=O)C1=NOC(c2ccccc2)(c2ccccc2)C1]

In some aspects, the azole carboxylate safener can comprise mefenpyr, shown below, or an agriculturally acceptable salt or ester thereof. Mefenpyr's safening activity is described in The Pesticide Manual, Eighteenth Edition, 2016.

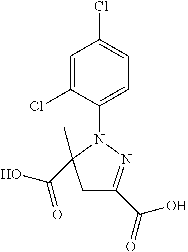 [SMILES: CC1(C(=O)O)CC(C(=O)O)=NN1c1ccc(Cl)cc1Cl]

In some aspects, the mefenpyr is provided as an agriculturally acceptable salt or ester. An exemplary agriculturally acceptable ester of mefenpyr is mefenpyr-diethyl, shown below.

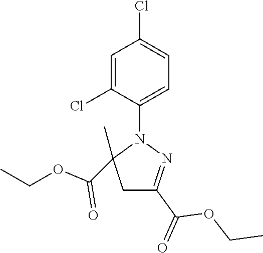 [SMILES: CCOC(=O)C1=NN(c2ccc(Cl)cc2Cl)C(C)(C(=O)OCC)C1]

Azole carboxylate safeners include, for example, isoxadifen and agriculturally acceptable salts and esters thereof. Isoxadifen has the following chemical structure.

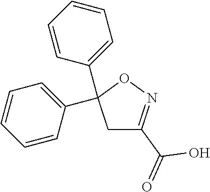 [SMILES: O=C(O)C1=NOC(c2ccccc2)(c2ccccc2)C1]

This form of isoxadifen may also be referred to as “isoxadifen-acid.”

In some aspects, the safener may comprise isoxadifen-acid, an agriculturally acceptable salt of isoxadifen, an agriculturally acceptable ester of isoxadifen, or mixtures thereof. An exemplary agriculturally acceptable ester of isoxadifen is isoxadifen-ethyl, shown below.

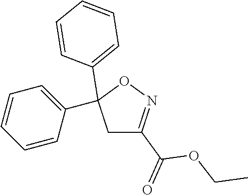 [SMILES: CCOC(=O)C1=NOC(c2ccccc2)(c2ccccc2)C1]

In some aspects, the azole carboxylate safener can be provided as an agriculturally acceptable ester. Suitable esters include, but are not limited to, methyl, ethyl, isopropyl, butyl, hexyl, heptyl, isoheptyl, mexyl, isooctyl, 2-ethylhexyl and butoxyethyl esters, and aryl esters such as benzyl. Exemplary agriculturally acceptable esters of the azole carboxylate safeners described herein include methyl, ethyl, and diethyl esters.

In some aspects, the azole carboxylate safener can comprise an agriculturally acceptable salt of the azole carboxylate safener. Agriculturally acceptable salts of the azole carboxylate safener include, for example sodium, potassium, ammonium, monoethanolammonium, diethanolammonium, triethanolammonium, monoisopropanolammonium, diisopropanolammonium, triisopropanolammonium, choline, N,N-dimethylethanolammonium, diethylammonium, dimethylammonium, trimethylammonium, triethylammonium, and isopropylammonium salts of the azole carboxylate safener.

An azole carboxylate safener, when applied in combination with herbicides, can be used to reduce phytotoxicity to crops such as wheat, barley, triticale, rye, teff, oats, maize, sorghum, millet, rice, millet, canola/oilseed rape, flax, soy, sunflower, sugar beet, cotton, sugarcane, and pasture grasses.

The azole carboxylate safener or an agriculturally acceptable salt or ester thereof can be used in an amount sufficient to induce a safening effect. In some aspects, the azole carboxylate safener is selected from the group consisting of fenchlorazole, fenclorim, flurazole, furilazole, isoxadifen, mefenpyr, an agriculturally acceptable salt or ester thereof, or combinations thereof, and is applied to vegetation or an area adjacent the vegetation or applied to soil or water in an amount of 1 g active ingredient per hectare (ai/ha) or greater, such as 2 g ai/ha or greater, 3 g ai/ha or greater, 4 g ai/ha or greater, 5 g ai/ha or greater, 6 g ai/ha or greater, 7 g ai/ha or greater, 8 g ai/ha or greater, 9 g ai/ha or greater, 10 g ai/ha or greater, 11 g ai/ha or greater, 12 g ai/ha or greater, 13 g ai/ha or greater, 14 g ai/ha or greater, 15 g ai/ha or greater, 16 g ai/ha or greater, 17 g ai/ha or greater, 18 g ai/ha or greater, 19 g ai/ha or greater, 20 g ai/ha or greater, 22 g ai/ha or greater, 24 g ai/ha or greater, 25 g ai/ha or greater, 26 g ai/ha or greater, 28 g ai/ha or greater, 30 g ai/ha or greater, 32 g ai/ha or greater, 34 g ai/ha or greater, 35 g ai/ha or greater, 36 g ai/ha or greater, 38 g ai/ha or greater, 40 g ai/ha or greater, 42.5 g ai/ha or greater, 45 g ai/ha or greater, 47.5 g ai/ha or greater, 50 g ai/ha or greater, 52.5 g ai/ha or greater, 55 g ai/ha or greater, 57.5 g ai/ha or greater, 60 g ai/ha or greater, 65 g ai/ha or greater, 70 g ai/ha or greater, 75 g ai/ha or greater, 80 g ai/ha or greater, 85 g ai/ha or greater, 90 g ai/ha or greater, 95 g ai/ha or greater, 100 g ai/ha or greater, 110 g ai/ha or greater, 120 g ai/ha or greater, 130 g ai/ha or greater, 140 g ai/ha or greater, 150 g ai/ha or greater, 160 g ai/ha or greater, 170 g ai/ha or greater, 180 g ai/ha or greater, 190 g ai/ha or greater, 200 g ai/ha or greater, 210 g ai/ha or greater, 220 g ai/ha or greater, 230 g ai/ha or greater, 240 g ai/ha or greater, 250 g ai/ha or greater, 260 g ai/ha or greater, 270 g ai/ha or greater, 280 g ai/ha or greater, or 290 g ai/ha or greater; in an amount of 300 g ai/ha or less, such as 290 g ai/ha or less, 280 g ai/ha or less, 270 g ai/ha or less, 260 g a/ha or less, 250 g ai/ha or less, 240 g ai/ha or less, 230 g ai/ha or less, 220 g ai/ha or less, 210 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 57.5 g ai/ha or less, 55 g ai/ha or less, 52.5 g ai/ha or less, 50 g ai/ha or less, 47.5 g ai/ha or less, 45 g ai/ha or less, 42.5 g ai/ha or less, 40 g ai/ha or less, 38 g ai/ha or less, 36 g ai/ha or less, 35 g ai/ha or less, 34 g ai/ha or less, 32 g ai/ha or less, 30 g ai/ha or less, 28 g ai/ha or less, 26 g ai/ha or less, 25 g ai/ha or less, 24 g ai/ha or less, 22 g ai/ha or less, 20 g ai/ha or less, 19 g ai/ha or less, 18 g ai/ha or less, 17 g ai/ha or less, 16 g ai/ha or less, 15 g ai/ha or less, 14 g ai/ha or less, 13 g ai/ha or less, 12 g ai/ha or less, 11 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, or 2 g ai/ha or less; or in an amount within any range defined between any pair of the preceding values, such as from 1 g ai/ha to 300 g ai/ha, from 5 g ai/ha to 150 g ai/ha, from 10 g ai/ha to 200 g ai/ha, from 20 g ai/ha to 75 g ai/ha, or from 40 g ai/ha to 100 g ai/ha.

### Formulations

The present disclosure also includes formulations of the compositions and methods disclosed herein.

### Additives

The compositions and methods disclosed herein can also be mixed with or applied with an additive. In some aspects, the additive is added sequentially. In some aspects, the additive is added simultaneously. In some aspects, the additive is premixed with the compounds according to Formula I. In some aspects, the additive is premixed with the azole carboxylate safener or agriculturally acceptable salt or ester thereof.

### Other Pesticides

Some aspects of the disclosed safened compositions disclosed include adding one or more additional pesticide active ingredients to the safened compositions. These pesticide active ingredients may include one or more of an herbicide, an insecticide, a fungicide, a nematocide, a miticide, a arthropodicide, a bactericide, a plant growth regulator, or combinations thereof that are compatible with the compositions of the present disclosure.

In some aspects, the additive is an additional herbicide. For example, the compositions described herein can be applied in conjunction with one or more additional herbicides to control undesirable vegetation. The composition can be formulated with the one or more additional herbicides, tank mixed with the one or more additional herbicides, or applied sequentially with the one or more additional herbicides, such as the exemplary herbicides described below.

### ALS Inhibitors

In addition to the compounds according to Formula I, the compositions can include an acetolactate synthase (ALS) inhibitor. ALS inhibitors disrupt the production of amino acids in the plant, which eventually leads to inhibition of DNA synthesis. Examples of ALS inhibitors include sulfonylureas, imidazolinones, triazolopyrimidine sulfonamides, pyrimidinyl oxybenzoates and sulfonylaminocarbonyl triazolinones. In some aspects, the ALS inhibitor can contain a triazolopyrimidine sulfonamide herbicide. In some aspects, the ALS inhibitor can contain an imidazolinone herbicide. In some aspects, the ALS inhibitor can contain a pyrimidinyl oxybenzoate herbicide. In some aspects, the ALS inhibitor can contain a sulfonylaminocarbonyl triazolinone herbicide. In some aspects, the ALS inhibitor can contain a sulfonylurea herbicide.

In some aspects, the composition can include an ALS inhibitor selected from the group of imidazolinones, triazolopyrimidine sulfonamides, pyrimidinyl oxybenzoates, sulfonylaminocarbonyl triazolinones, sulfonylureas, and combinations thereof. In some cases, the composition can include amidosulfuron, azimsulfuron, bispyribac, bensulfuron, chlorimuron, chlorsulfuron, cinosulfuron, cloransulam, cyclosulfamuron, diclosulam, ethametsulfuron, ethoxysulfuron, flazasulfuron, florasulam, flucarbazone, flucetosulfuron, flumetsulam, flupyrsulfuron, foramsulfuron, halosulfuron, imazamethabenz, imazamox, imazapic, imazapyr, imazaquin, imazethapyr, imazosulfuron, iodosulfuron, iofensulfuron, mesosulfuron, metazosulfuron, metosulam, metsulfuron, nicosulfuron, orthosulfamuron, oxasulfuron, penoxsulam, primisulfuron, propoxycarbazone, propyrisulfuron, prosulfuron, pyrazosulfuron, pyribenzoxim, pyriftalid, pyriminobac, pyrimisulfan, pyrithiobac, pyroxsulam, rimsulfuron, sulfometuron, sulfosulfuron, thiencarbazone, thifensulfuron, triasulfuron, tribenuron, trifloxysulfuron, triflusulfuron, tritosulfuron, agriculturally acceptable salts and esters thereof, and combinations thereof.

The ALS inhibitor or agriculturally acceptable salt or ester thereof can be applied to vegetation or an area adjacent the vegetation or applied to soil or water to prevent the emergence or growth of vegetation in an amount sufficient to induce a herbicidal effect. In some aspects, the ALS inhibitor or agriculturally acceptable salt or ester thereof is applied to vegetation or an area adjacent the vegetation or applied to soil or water to prevent the emergence or growth of vegetation in an amount of 0.25 grams active ingredient per hectare (g ai/ha) or more, such as 0.3 g ai/ha or more, 0.4 g ai/ha or more, 0.5 g ai/ha or more, 0.6 g ai/ha or more, 0.7 g ai/ha or more, 0.8 g ai/ha or more, 0.9 g ai/ha or more, 1 g ai/ha or more, 1.5 g ai/ha or more, 2 g ai/ha or more, 2.5 g ai/ha or more, 3 g ai/ha or more, 3.5 g ai/ha or more, 4 g ai/ha or more, 4.5 g ai/ha or more, 5 g ai/ha or more, 6 g ai/ha or more, 7 g ai/ha or more, 8 g ai/ha or more, 9 g ai/ha or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 250 g ai/ha or more, 260 g ai/ha or more, 280 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1550 g ai/ha or more, 1600 g ai/ha or more, 1650 g ai/ha or more, 1675 g ai/ha or more, 1680 g ai/ha or more, or 1690 g ai/ha or more; in an amount of 1700 g ai/ha or less, such as 1690 g ai/ha or less, 1680 g ai/ha or less, 1675 g ai/ha or less, 1650 g ai/ha or less, 1600 g ai/ha or less, 1550 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 350 g ai/ha or less, 340 g ai/ha or less, 320 g ai/ha or less, 300 g ai/ha or less, 280 g ai/ha or less, 260 g ai/ha or less, 250 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9 g ai/ha or less, 8 g ai/ha or less, 7 g ai/ha or less, 6 g ai/ha or less, 5 g ai/ha or less, 4.5 g ai/ha or less, 4 g ai/ha or less, 3.5 g ai/ha or less, 3 g ai/ha or less, 2.5 g ai/ha or less, 2 g ai/ha or less, 1.5 g ai/ha or less, 1 g ai/ha or less, 0.9 g ai/ha or less, 0.8 g ai/ha or less, 0.7 g ai/ha or less, 0.6 g ai/ha or less, 0.5 g ai/ha or less, 0.4 g ai/ha or less, or 0.3 g ai/ha or less; or in an amount ranging from any of the minimum values described above to any of the maximum values described above, such as 0.25-1700 g ai/ha, 0.25-1250 g ai/ha, 0.5-150 g ai/ha, 2-90 g ai/ha, 2.5-200 g ai/ha, 3-900 g ai/ha, 5-260 g ai/ha, 5-750 g ai/ha, 5-1000 g ai/ha, 6-280 g ai/ha, 7-100 g ai/ha, 10-560 g ai/ha, 10-1600 g ai/ha, 20-500 g ai/ha, 25-140 g ai/ha, 30-480 g ai/ha, 40-400 g ai/ha, 50-320 g ai/ha, 60-300 g ai/ha, 70-1250 g ai/ha, 100-140 g ai/ha, 140-520 g ai/ha, or 250-1700 g ai/ha.

### Triazolopyrimidine Sulfonamide Herbicides

In some aspects, the composition contains a herbicidally effective amount of compounds according to Formula I, and (b) florasulam, cloransulam, diclosulam, flumetsulam, metosulam, penoxsulam, or pyroxsulam, or agriculturally acceptable salts or esters thereof.

### Florasulam

Compositions and methods of the present disclosure can include florasulam or an agriculturally acceptable salt or ester thereof. Florasulam, shown below, is a triazolopyrimidine sulfonamide that provides broad-spectrum control of many broad-leaved weeds in cereals and maize. Florasulam, as well as methods of preparing florasulam, are known in the art. Its herbicidal activity is described, for example, in The Pesticide Manual, Sixteenth Edition, 2012.

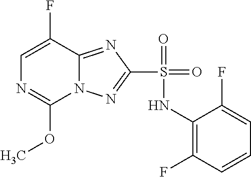 [SMILES: COc1ncc(F)c2nc(S(=O)(=O)Nc3c(F)cccc3F)nn12]

Florasulam can be applied to vegetation or an area adjacent the vegetation or applied to soil or water to prevent the emergence or growth of vegetation in an amount sufficient to induce a herbicidal effect. In some aspects, florasulam is applied to vegetation or an area adjacent the vegetation or applied to soil or water to prevent the emergence or growth of vegetation in an amount of 0.25 g ai/ha or more, such as 0.3 g ai/ha or more, 0.4 g ai/ha or more, 0.5 g ai/ha or more, 0.6 g ai/ha or more, 0.7 g ai/ha or more, 0.8 g ai/ha or more, 0.9 g ai/ha or more, 1 g ai/ha or more, 1.1 g ai/ha or more, 1.25 g ai/ha or more, 1.5 g ai/ha or more, 1.75 g ai/ha or more, 2 g ai/ha or more, 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.25 g ai/ha or more, 4.5 g ai/ha or more, 4.75 g ai/ha or more, 5 g ai/ha or more, 5.25 g ai/ha or more, 5.5 g ai/ha or more, 5.75 g ai/ha or more, 6 g ai/ha or more, 6.25 g ai/ha or more, 6.5 g ai/ha or more, 6.75 g ai/ha or more, 7 g ai/ha or more, 7.25 g ai/ha or more, 7.5 g ai/ha or more, 8 g ai/ha or more, 8.5 g ai/ha or more, 9 g ai/ha or more, or 9.5 g ai/ha, or more; in an amount of 10 g ai/ha or less, such as 9.5 g ai/ha or less, 9 g ai/ha or less, 8.5 g ai/ha or less, 8 g ai/ha or less 7.5 g ai/ha or less, 7.25 g ai/ha or less, 7 g ai/ha or less, 6.75 g ai/ha or less, 6.5 g ai/ha or less, 6.25 g ai/ha or less, 6 g ai/ha or less, 5.75 g ai/ha or less, 5.5 g ai/ha or less, 5.25 g ai/ha or less, 5 g ai/ha or less, 4.75 g ai/ha or less, 4.5 g ai/ha or less, 4.25 g ai/ha or less, 4 g ai/ha or less, 3.75 g ai/ha or less, 3.5 g ai/ha or less, 3.25 g ai/ha or less, 3 g ai/ha or less, 2.75 g ai/ha or less, 2.5 g ai/ha or less, 2.25 g ai/ha or less, 2 g ai/ha or less, 1.75 g ai/ha or less, 1.5 g ai/ha or less, 1.25 g ai/ha or less, 1 g ai/ha or less, 0.9 g ai/ha or less, 0.8 g ai/ha or less, 0.7 g ai/ha or less, 0.6 g ai/ha or less, 0.5 g ai/ha or less, 0.4 g ai/ha or less, or 0.3 g ai/ha or less; or in an amount ranging from any of the minimum values described above to any of the maximum values described above, such as 0.25-10 g ai/ha, 0.25-7 g ai/ha, 0.4-6.5 g ai/ha, 0.5-6 g ai/ha, 0.6-9 g ai/ha, 0.7-8.5 g ai/ha, 0.8-4.5 g ai/ha, 1-4 g ai/ha, 1.1-7.5 g ai/ha, 1.5-5.75 g ai/ha, 2-7.25 g ai/ha, 2.25-10 g ai/ha, 3-8.5 g ai/ha, or 3.5-9.5 g ai/ha.

### Cloransulam

Compositions and methods of the present disclosure can include cloransulam or an agriculturally acceptable salt or ester thereof. Cloransulam, as well as methods of preparing cloransulam, are known in the art. In some aspects, cloransulam can be provided as an agriculturally acceptable ester of cloransulam. Cloransulam-methyl, shown below, is a

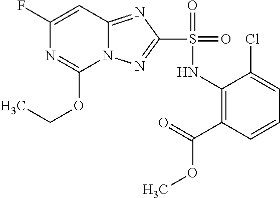 [SMILES: CCOc1nc(F)cc2nc(S(=O)(=O)Nc3c(Cl)cccc3C(=O)OC)nn12]

triazolopyrimidine sulfonamide that provides broad-spectrum control of many broadleaf weeds in soybeans and other broadleaf crops. Its herbicidal activity is described in The Pesticide Manual, Seventeenth Edition, 2016.

Cloransulam can be applied to vegetation or an area adjacent the vegetation or applied to soil or water to prevent the emergence or growth of vegetation in an amount sufficient to induce a herbicidal effect. In some aspects, cloransulam is applied to vegetation or an area adjacent the vegetation or applied to soil or water to prevent the emergence or growth of vegetation in an amount of 2.18 g ai/ha or more, such as 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.5 g ai/ha or more, 5 g ai/ha or more, 6 g ai/ha or more, 7 g ai/ha or more, 8 g ai/ha or more, 9 g ai/ha or more, 10 g ai/ha or more, 12 g ai/ha or more, 14 g ai/ha or more, 16 g ai/ha or more, 18 g ai/ha or more, 20 g ai/ha or more, 25 g ai/ha or more, 30 g ai/ha or more, 35 g ai/ha or more, 36 g ai/ha or more, 38 g ai/ha or more, 40 g ai/ha or more, 42 g ai/ha or more, 44 g ai/ha or more, 46 g ai/ha or more, 48 g ai/ha or more, 50 g ai/ha or more, 51 g ai/ha or more, 52 g ai/ha or more, 53 g ai/ha or more, 54 g ai/ha or more, 55 g ai/ha or more, 56 g ai/ha or more, 57 g ai/ha or more, 58 g ai/ha or more, or 59 g ai/ha or more; in an amount of 61 g ai/ha or less, such as 60 g ai/ha or less, 59 g ai/ha or less, 58 g ai/ha or less, 57 g ai/ha or less, 56 g ai/ha or less, 55 g ai/ha or less, 54 g ai/ha or less, 53 g ai/ha or less, 52 g ai/ha or less, 51 g ai/ha or less, 50 g ai/ha or less, 48 g ai/ha or less, 46 g ai/ha or less, 44 g ai/ha or less, 42 g ai/ha or less, 40 g ai/ha or less, 38 g ai/ha or less, 36 g ai/ha or less, 35 g ai/ha or less, 30 g ai/ha or less, 25 g ai/ha or less, 20 g ai/ha or less, 18 g ai/ha or less, 16 g ai/ha or less, 14 g ai/ha or less, 12 g ai/ha or less, 10 g ai/ha or less, 9 g ai/ha or less, 8 g ai/ha or less, 7 g ai/ha or less, 6 g ai/ha or less, 5 g ai/ha or less, 4.5 g ai/ha or less, 4 g ai/ha or less, 3.75 g ai/ha or less, 3.5 g ai/ha or less, 3.25 g ai/ha or less, 3 g ai/ha or less, 2.75 g ai/ha or less, 2.5 g ai/ha or less, or 2.25 g ai/ha or less; or

in an amount ranging from any of the minimum values described above to any of the maximum values described above, such as 2.18-61 g ai/ha, 2.5-60 g ai/ha, 2.75-44 g ai/ha, 3-59 g ai/ha, 3.25-60 g ai/ha, 3.5-40 g ai/ha, 3.75-18 g ai/ha, 3.75-50 g ai/ha, 4-60 g ai/ha, 4-52 g ai/ha, 4.25-30 g ai/ha, 4.25-55 g ai/ha, 4.5-51 g ai/ha, or 5-38 g ai/ha.

### Diclosulam

Compositions and methods of the present disclosure can include diclosulam or an agriculturally acceptable salt or ester thereof. Diclosulam, shown below, is a triazolopyrimidine sulfonamide that provides broad-spectrum control of many broadleaf weeds in soybeans and peanuts. Diclosulam, as well as methods of preparing diclosulam, are known in the art. Its herbicidal activity is described in The Pesticide Manual, Seventeenth Edition, 2016.

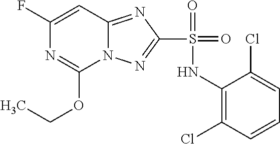 [SMILES: CCOc1nc(F)cc2nc(S(=O)(=O)Nc3c(Cl)cccc3Cl)nn12]

Diclosulam can be applied to vegetation or an area adjacent the vegetation or applied to soil or water to prevent the emergence or growth of vegetation in an amount sufficient to induce a herbicidal effect. In some aspects, diclosulam is applied to vegetation or an area adjacent the vegetation or applied to soil or water to prevent the emergence or growth of vegetation in an amount of 2.18 g ai/ha or more, such as 2.25 g ai/ha or more, 2.5 g ai/ha or more, 2.75 g ai/ha or more, 3 g ai/ha or more, 3.25 g ai/ha or more, 3.5 g ai/ha or more, 4 g ai/ha or more, 4.5 g ai/ha or more, 5 g ai/ha or more, 6 g ai/ha or more, 7 g ai/ha or more, 8 g ai/ha or more, 9 g ai/ha or more, 10 g ai/ha or more, 12 g ai/ha or more, 14 g ai/ha or more, 16 g ai/ha or more, 18 g ai/ha or more, 20 g ai/ha or more, 22 g ai/ha or more, 24 g ai/ha or more, 26 g ai/ha or more, 27 g ai/ha or more, 28 g ai/ha or more, 29 g ai/ha or more, 30 g ai/ha or more, 31 g ai/ha or more, 31.5 g ai/ha or more, 32 g ai/ha or more, 32.5 g ai/ha or more, 33 g ai/ha or more, 33.5 g ai/ha or more, 34 g ai/ha or more, 34.5 g ai/ha or more, or 34.75 g ai/ha or more; in an amount of 35 g ai/ha or less, such as 34.5 g ai/ha or less, 34 g ai/ha or less, 33.5 g ai/ha or less, 33 g ai/ha or less, 32.5 g ai/ha or less, 32 g ai/ha or less, 31.5 g ai/ha or less, 31 g ai/ha or less, 30 g ai/ha or less, 29 g ai/ha or less, 28 g ai/ha or less, 27 g ai/ha or less, 26 g ai/ha or less, 24 g ai/ha or less, 22 g ai/ha or less, 20 g ai/ha or less, 18 g ai/ha or less, 16 g ai/ha or less, 14 g ai/ha or less, 12 g ai/ha or less, 10 g ai/ha or less, 9 g ai/ha or less, 8 g ai/ha or less, 7 g ai/ha or less, 6 g ai/ha or less, 5 g ai/ha or less, 4.5 g ai/ha or less, 4 g ai/ha or less, 3.5 g ai/ha or less, 2.75 g ai/ha or less, 2.5 g ai/ha or less, 2.25 g ai/ha or less; or in an amount ranging from any of the minimum values described above to any of the maximum values described above, such as 2.18-35 g ai/ha, 2.5-34 g ai/ha, 2.75-22 g ai/ha, 3-29 g ai/ha, 3.25-34 g ai/ha, 3.25-32 g ai/ha, 3.5-30 g ai/ha, 3.75-27 g ai/ha, 4-35 g ai/ha, 4-30 g ai/ha, 4.5-18 g ai/ha, 4.5-33.5 g ai/ha, 5-32 g ai/ha, or 6-33 g ai/ha.

### Flumetsulam

Compositions and methods of the present disclosure can include flumetsulam or an agriculturally acceptable salt or ester thereof. Flumetsulam, shown below, is a triazolopyrimidine sulfonamide that provides control of broadleaf weeds and greens in soybeans, field peas, and maize. Flumetsulam, as well as methods of preparing flumetsulam, are known in the art. Its herbicidal activity is described in The Pesticide Manual, Seventeenth Edition, 2016.

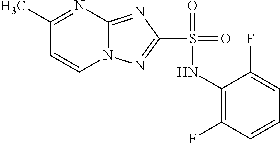 [SMILES: Cc1ccn2nc(S(=O)(=O)Nc3c(F)cccc3F)nc2n1]

Flumetsulam can be applied to vegetation or an area adjacent the vegetation or applied to soil or water to prevent the emergence or growth of vegetation in an amount sufficient to induce a herbicidal effect. In some aspects, flumetsulam is applied to vegetation or an area adjacent the vegetation or applied to soil or water to prevent the emergence or growth of vegetation in an amount of 2.18 g ai/ha or more, such as 2.5 g ai/ha or more, 2.75 g ai/ha or more, 3 g ai/ha or more, 3.5 g ai/ha or more, 4 g ai/ha or more, 4.5 g ai/ha or more, 5 g ai/ha or more, 7 g ai/ha or more, 9 g ai/ha or more, 10 g ai/ha or more, 15 g ai/ha or more, 20 g ai/ha or more, 25 g ai/ha or more, 30 g ai/ha or more, 35 g ai/ha or more, 36 g ai/ha or more, 37 g ai/ha or more, 38 g ai/ha or more, 39 g ai/ha or more, 40 g ai/ha or more, 41 g ai/ha or more, 42 g ai/ha or more, 44 g ai/ha or more, 46 g ai/ha or more, 48 g ai/ha or more, 50 g ai/ha or more, 52 g ai/ha or more, 54 g ai/ha or more, 56 g ai/ha or more, 58 g ai/ha or more, 60 g ai/ha or more, 62 g ai/ha or more, 64 g ai/ha or more, 66 g ai/ha or more, 68 g ai/ha or more, 70 g ai/ha or more, 72 g ai/ha or more, 74 g ai/ha or more, or 77 g ai/ha or more; in an amount of 78 g ai/ha or less, such as 77 g ai/ha or less, 75 g ai/ha or less, 74 g ai/ha or less, 72 g ai/ha or less, 70 g ai/ha or less, 68 g ai/ha or less, 66 g ai/ha or less, 64 g ai/ha or less, 62 g ai/ha or less, 60 g ai/ha or less, 58 g ai/ha or less, 56 g ai/ha or less, 54 g ai/ha or less, 52 g ai/ha or less, 50 g ai/ha or less, 48 g ai/ha or less, 46 g ai/ha or less, 44 g ai/ha or less, 42 g ai/ha or less, 41 g ai/ha or less, 40 g ai/ha or less, 39 g ai/ha or less, 38 g ai/ha or less, 37 g ai/ha or less, 36 g ai/ha or less, 35 g ai/ha or less, 30 g ai/ha or less, 25 g ai/ha or less, 20 g ai/ha or less, 15 g ai/ha or less, 10 g ai/ha or less, 9 g ai/ha or less, 7 g ai/ha or less, 5 g ai/ha or less, 4.5 g ai/ha or less, 4 g ai/ha or less, 3.5 g ai/ha or less, 3 g ai/ha or less, 2.75 g ai/ha or less, 2.5 g ai/ha or less; or in an amount ranging from any of the minimum values described above to any of the maximum values described above, such as 2.18-78 g ai/ha, 2.5-75 g ai/ha, 2.75-54 g ai/ha, 3-46 g ai/ha, 3-78 g ai/ha, 3.5-39 g ai/ha, 3.5-72 g ai/ha, 4-60 g ai/ha, 4-75 g ai/ha, 4.5-25 g ai/ha, 4.5-66 g ai/ha, 5-58 g ai/ha, or 9-62 g ai/ha.

### Penoxsulam

Compositions and methods of the present disclosure can include penoxsulam or an agriculturally acceptable salt or ester thereof. Penoxsulam, shown below, is a triazolopyrimidine sulfonamide that provides control of Echinochloa spp., as well as many broadleaf, sedge, and aquatic weeds in rice. Penoxsulam, as well as methods of preparing penoxsulam, are known in the art. Its herbicidal activity is described in The Pesticide Manual, Seventeenth Edition, 2016.

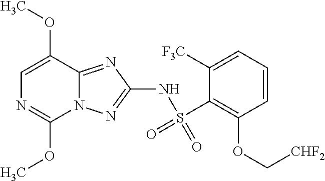 [SMILES: COc1cnc(OC)n2nc(NS(=O)(=O)c3c(OCC(F)F)cccc3C(F)(F)F)nc12]

Penoxsulam can be applied to vegetation or an area adjacent the vegetation or applied to soil or water to prevent the emergence or growth of vegetation in an amount sufficient to induce a herbicidal effect. In some aspects, penoxsulam is applied to vegetation or an area adjacent the vegetation or applied to soil or water to prevent the emergence or growth of vegetation in an amount of 2.5 g ai/ha or more, such as 2.75 g ai/ha or more, 3 g ai/ha or more, 3.5 g ai/ha or more, 4 g ai/ha or more, 4.5 g ai/ha or more, 5 g ai/ha or more, 6 g ai/ha or more, 7 g ai/ha or more, 8 g ai/ha or more, 9 g ai/ha or more, 10 g ai/ha or more, 12 g ai/ha or more, 14 g ai/ha or more, 16 g ai/ha or more, 18 g ai/ha or more, 20 g ai/ha or more, 22 g ai/ha or more, 24 g ai/ha or more, 26 g ai/ha or more, 28 g ai/ha or more, 30 g ai/ha or more, 32 g ai/ha or more, 34 g ai/ha or more, 36 g ai/ha or more, 38 g ai/ha or more, 40 g ai/ha or more, 41 g ai/ha or more, 42 g ai/ha or more, 43 g ai/ha or more, 44 g ai/ha or more, 45 g ai/ha or more, 46 g ai/ha or more, 47 g ai/ha or more, 48 g ai/ha or more, or 49 g ai/ha or more; in an amount of 50 g ai/ha or less, such as 49 g ai/ha or less, 48 g ai/ha or less, 47 g ai/ha or less, 46 g ai/ha or less, 45 g ai/ha or less, 44 g ai/ha or less, 43 g ai/ha or less, 42 g ai/ha or less, 41 g ai/ha or less, 40 g ai/ha or less, 38 g ai/ha or less, 36 g ai/ha or less, 34 g ai/ha or less, 32 g ai/ha or less, 30 g ai/ha or less, 28 g ai/ha or less, 26 g ai/ha or less, 24 g ai/ha or less, 22 g ai/ha or less, 20 g ai/ha or less, 18 g ai/ha or less, 16 g ai/ha or less, 14 g ai/ha or less, 12 g ai/ha or less, 10 g ai/ha or less, 9 g ai/ha or less, 8 g ai/ha or less, 7 g ai/ha or less, 6 g ai/ha or less, 5 g ai/ha or less, 4.75 g ai/ha or less, 4.5 g ai/ha or less, 4.25 g ai/ha or less, 4 g ai/ha or less, 3.5 g ai/ha or less, 3 g ai/ha or less, or 2.75 g ai/ha or less; or in an amount ranging from any of the minimum values described above to any of the maximum values described above, such as 2.5-50 g ai/ha, 3-28 g ai/ha, 3.5-40 g ai/ha, 4-34 g ai/ha, 4.25-50 g ai/ha, 4.5-47 g ai/ha, 5-45 g ai/ha, 6-36 g ai/ha, 8-22 g ai/ha, or 10-49 g ai/ha.

### Pyroxsulam

Compositions and methods of the present disclosure can include pyroxsulam or an agriculturally acceptable salt or ester thereof. Pyroxsulam, shown below, is a triazolopyrimidine sulfonamide that provides broad spectrum post-emergence annual grass and broadleaf weeds control in cereals. Pyroxsulam, as well as methods of preparing pyroxsulam, are known in the art. Its herbicidal activity is described in The Pesticide Manual, Seventeenth Edition, 2016.

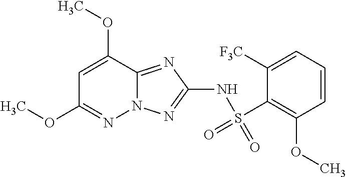

Pyroxsulam can be applied to vegetation or an area adjacent the vegetation or applied to soil or water to prevent the emergence or growth of vegetation in an amount sufficient to induce a herbicidal effect. In some aspects, pyroxsulam is applied to vegetation or an area adjacent the vegetation or applied to soil or water to prevent the emergence or growth of vegetation in an amount of 1.9 g ai/ha or more, such as 2 g ai/ha or more, 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.5 g ai/ha or more, 5 g ai/ha or more, 6 g ai/ha or more, 7 g ai/ha or more, 8 g ai/ha or more, 9 g ai/ha or more, 9.5 g ai/ha or more, 10 g ai/ha or more, 10.5 g ai/ha or more, 11 g ai/ha or more, 11.5 g ai/ha or more, 12 g ai/ha or more, 12.5 g ai/ha or more, 13 g ai/ha or more, 13.5 g ai/ha or more, 14 g ai/ha or more, 14.5 g ai/ha or more, 15 g ai/ha or more, 15.25 g ai/ha or more, 15.5 g ai/ha or more, 15.75 g ai/ha or more, 16 g ai/ha or more, 16.25 g ai/ha or more, 16.5 g ai/ha or more, 16.75 g ai/ha or more, 17 g ai/ha or more, 17.25 g ai/ha or more, 17.5 g ai/ha or more, 17.75 g ai/ha or more, 18 g ai/ha or more, 18.25 g ai/ha or more, or 18.5 g ai/ha or more; in an amount of 18.75 g ai/ha or less, such as 18.5 g ai/ha or less, 18.25 g ai/ha or less, 18 g ai/ha or less, 17.75 g ai/ha or less, 17.5 g ai/ha or less, 17.25 g ai/ha or less, 17 g ai/ha or less, 16.75 g ai/ha or less, 16.5 g ai/ha or less, 16.25 g ai/ha or less, 16 g ai/ha or less, 15.75 g ai/ha or less, 15.5 g ai/ha or less, 15.25 g ai/ha or less, 15 g ai/ha or less, 14.5 g ai/ha or less, 14 g ai/ha or less, 13.5 g ai/ha or less, 13 g ai/ha or less, 12.5 g ai/ha or less, 12 g ai/ha or less, 11.5 g ai/ha or less, 11 g ai/ha or less, 10.5 g ai/ha or less, 10 g ai/ha or less, 9.5 g ai/ha or less, 9 g ai/ha or less, 8 g ai/ha or less, 7 g ai/ha or less, 6 g ai/ha or less, 5 g ai/ha or less, 4.5 g ai/ha or less, 4 g ai/ha or less, 3.5 g ai/ha or less, 3.25 g ai/ha or less, 3 g ai/ha or less, 2.75 g ai/ha or less, 2.5 g ai/ha or less, or 2.25 g ai/ha or less; or in an amount ranging from any of the minimum values described above to any of the maximum values described above, such as 1.9-18.75 g ai/ha, 2.25-18 g ai/ha, 2.5-14.5 g ai/ha, 2.75-12.5 g ai/ha, 2.75-18.5 g ai/ha, 3-13 g ai/ha, 3.25-10.5 g ai/ha, 3.5-16 g ai/ha, 4-15.25 g ai/ha, 5-18 g ai/ha, 6-17.75 g ai/ha, 8-16 g ai/ha, 9-18 g ai/ha, or 10-14.5 g ai/ha.

### Imidazolinone Herbicides

In some aspects, the composition contains a herbicidally effective amount of compounds according to Formula I, and (b)imazamethabenz, imazamox, imazapic, imazapyr, imazaquin, imazethapyr, or agriculturally acceptable salts or esters thereof, or combinations thereof.

### Imazamethabenz

Compositions and methods of the present disclosure can include imazamethabenz or an agriculturally acceptable salt or ester thereof. Imazamethabenz, shown below, is a mixture of isomers and an imidazolinone that provides post-emergence control of Avena species, Alopecurus myosuroides, Apera spica-venti and dicotyledonous weeds in wheat, barley, rye and sunflowers. Imazamethabenz, as well as methods of preparing imazamethabenz, are known in the art. Its herbicidal activity is described in The Pesticide Manual, Seventeenth Edition, 2016.

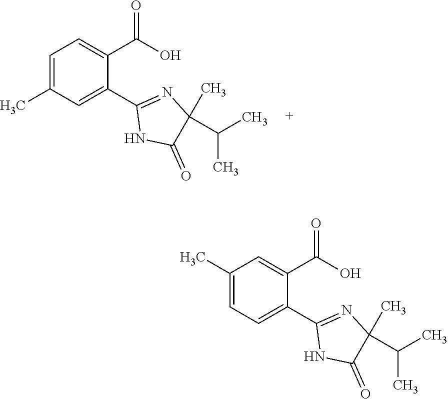

Imazamethabenz can be applied to vegetation or an area adjacent the vegetation or applied to soil or water to prevent the emergence or growth of vegetation in an amount sufficient to induce a herbicidal effect. In some aspects, imazamethabenz is applied to vegetation or an area adjacent the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 620 g ai/ha or more, 640 g ai/ha or more, 650 g ai/ha or more, 660 g ai/ha or more, 670 g ai/ha or more, 680 g ai/ha or more, or 690 g ai/ha or more; in an amount of 700 g ai/ha or less, such as 690 g ai/ha or less, 680 g ai/ha or less, 670 g ai/ha or less, 660 g ai/ha or less, 650 g ai/ha or less, 640 g ai/ha or less, 620 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 280 g ai/ha or less, 260 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-700 g ai/ha, 55-400 g ai/ha, 60-650 g ai/ha, 75-520 g ai/ha, 80-400 g ai/ha, 85-500 g ai/ha, 90-220 g ai/ha, 100-700 g ai/ha, 100-500 g ai/ha, 110-640 g ai/ha, 120-420 g ai/ha, 120-500 g ai/ha, 140-670 g ai/ha, or 150-580 g ai/ha.

### Imazamox

Compositions and methods of the present disclosure can include imazamox or an agriculturally acceptable salt or ester thereof. Imazamox, shown below, is an imidazolinone that provides pre- and post-emergence control of broadleaf and grass weeds, e.g., in rice, maize, rape, alfalfa, peas and beans. Imazamox, as well as methods of preparing imazamox, are known in the art. Its herbicidal activity is described in The Pesticide Manual, Seventeenth Edition, 2016.

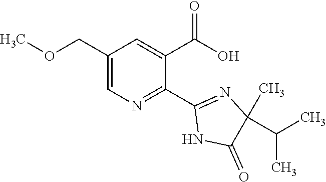 [SMILES: COCc1cnc(C2=NC(C)(C(C)C)C(=O)N2)c(C(=O)O)c1]

Imazamox can be applied to vegetation or an area adjacent the vegetation or applied to soil or water to prevent the emergence or growth of vegetation in an amount sufficient to induce a herbicidal effect. In some aspects, imazamox is applied to vegetation or an area adjacent the vegetation or applied to soil or water to prevent the emergence or growth of vegetation in an amount of 5 g ai/ha or more, such as 6 g ai/ha or more, 7 g ai/ha or more, 8 g ai/ha or more, 9 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 17 g ai/ha or more, 18 g ai/ha or more, 19 g ai/ha or more, 20 g ai/ha or more, 21 g ai/ha or more, 22 g ai/ha or more, 23 g ai/ha or more, 24 g ai/ha or more, 25 g ai/ha or more, 26 g ai/ha or more, 27 g ai/ha or more, 28 g ai/ha or more, 29 g ai/ha or more, 30 g ai/ha or more, 32 g ai/ha or more, 34 g ai/ha or more, 36 g ai/ha or more, 38 g ai/ha or more, 40 g ai/ha or more, 44 g ai/ha or more, 46 g ai/ha or more, 48 g ai/ha or more, 50 g ai/ha or more, 52 g ai/ha or more, 54 g ai/ha or more, 56 g ai/ha or more, or 58 g ai/ha or more; in an amount of 60 g ai/ha or less, such as 58 g ai/ha or less, 56 g ai/ha or less, 54 g ai/ha or less, 52 g ai/ha or less, 50 g ai/ha or less, 48 g ai/ha or less, 46 g ai/ha or less, 44 g ai/ha or less, 42 g ai/ha or less, 40 g ai/ha or less, 38 g ai/ha or less, 36 g ai/ha or less, 34 g ai/ha or less, 32 g ai/ha or less, 30 g ai/ha or less, 29 g ai/ha or less, 28 g ai/ha or less, 27 g ai/ha or less, 26 g ai/ha or less, 25 g ai/ha or less, 24 g ai/ha or less, 23 g ai/ha or less, 22 g ai/ha or less, 21 g ai/ha or less, 20 g ai/ha or less, 19 g ai/ha or less, 18 g ai/ha or less, 17 g ai/ha or less, 16 g ai/ha or less, 15 g ai/ha or less, 14 g ai/ha or less, 13 g ai/ha or less, 12 g ai/ha or less, 11 g ai/ha or less, 10 g ai/ha or less, 9 g ai/ha or less, 8 g ai/ha or less, 7 g ai/ha or less, or 6 g ai/ha or less; or in an amount ranging from any of the minimum values described above to any of the maximum values described above, such as 5-60 g ai/ha, 5-52 g ai/ha, 6-46 g ai/ha, 6-54 g ai/ha, 7-38 g ai/ha, 7-50 g ai/ha, 8-48 g ai/ha, 9-45 g ai/ha, 10-40 g ai/ha, 10-58 g ai/ha, 11-36 g ai/ha, 12-54 g ai/ha, 13-60 g ai/ha, or 15-48 g ai/ha.

### Imazapic

Compositions and methods of the present disclosure can include imazapic or an agriculturally acceptable salt or ester thereof. Imazapic, shown below, is an imidazolinone that provides pre- and post-emergence control of a wide range of annual and perennial weeds in pasture, rangeland and non-cropland areas. Imazapic, as well as methods of preparing imazapic, are known in the art. Its herbicidal activity is described in The Pesticide Manual, Seventeenth Edition, 2016.

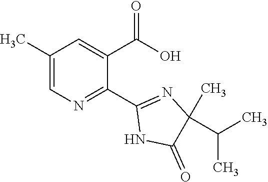 [SMILES: Cc1cnc(C2=NC(C)(C(C)C)C(=O)N2)c(C(=O)O)c1]

Imazapic can be applied to vegetation or an area adjacent the vegetation or applied to soil or water to prevent the emergence or growth of vegetation in an amount sufficient to induce a herbicidal effect. In some aspects, imazapic is applied to vegetation or an area adjacent the vegetation or applied to soil or water to prevent the emergence or growth of vegetation in an amount of 17.5 g ai/ha or more, such as 18 g ai/ha or more, 19 g ai/ha or more, 20 g ai/ha or more, 22 g ai/ha or more, 24 g ai/ha or more, 26 g ai/ha or more, 28 g ai/ha or more, 30 g ai/ha or more, 35 g ai/ha or more, 40 g ai/ha or more, 45 g ai/ha or more, 50 g ai/ha or more, 45 g ai/ha or more, 50 g ai/ha or more, 60 g ai/ha or more, 70 g ai/ha or more, 80 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 155 g ai/ha or more, 160 g ai/ha or more, 165 g ai/ha or more, 170 g ai/ha or more, 175 g ai/ha or more, 180 g ai/ha or more, 185 g ai/ha or more, 190 g ai/ha or more, 195 g ai/ha or more, 200 g ai/ha or more, 205 g ai/ha or more, 210 g ai/ha or more, or 215 g ai/ha or more; in an amount of 220 g ai/ha or less, such as 215 g ai/ha or less, 210 g ai/ha or less, 205 g ai/ha or less, 200 g ai/ha or less, 195 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 80 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 28 g ai/ha or less, 26 g ai/ha or less, 24 g ai/ha or less, 22 g ai/ha or less, 20 g ai/ha or less, 19 g ai/ha or less, or 18 g ai/ha or less; or in an amount ranging from any of the minimum values described above to any of the maximum values described above, such as 17.5-220 g ai/ha, 17.5-210 g ai/ha, 18-200 g ai/ha, 19-120 g ai/ha, 20-210 g ai/ha, 20-170 g ai/ha, 22-190 g ai/ha, 24-175 g ai/ha, 26-210 g ai/ha, 26-160 g ai/ha, 30-190 g ai/ha, 30-140 g ai/ha, 35-220 g ai/ha, or 45-215 g ai/ha.

### Imazapyr

Compositions and methods of the present disclosure can include imazapyr or an agriculturally acceptable salt or ester thereof. Imazapyr, shown below, is an imidazolinone that provides pre- and post-emergence control of annual and perennial grasses, broadleaf weeds, brush and trees. Imazapyr, as well as methods of preparing imazapyr, are known in the art. Its herbicidal activity is described in The Pesticide Manual, Seventeenth Edition, 2016.

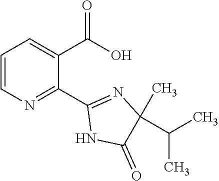 [SMILES: CC(C)C1(C)N=C(c2ncccc2C(=O)O)NC1=O]

Imazapyr can be applied to vegetation or an area adjacent the vegetation or applied to soil or water to prevent the emergence or growth of vegetation in an amount sufficient to induce a herbicidal effect. In some aspects, imazapyr is applied to vegetation or an area adjacent the vegetation or applied to soil or water to prevent the emergence or growth of vegetation in an amount of 100 g ai/ha or more, such as 120 g ai/ha or more, 125 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 600 g ai/ha or more, 700 g ai/ha or more, 800 g ai/ha or more, 900 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1550 g ai/ha or more, 1600 g ai/ha or more, 1620 g ai/ha or more, 1640 g ai/ha or more, 1660 g ai/ha or more, or 1680 g ai/ha or more; in an amount of 1700 g ai/ha or less (e.g., 1680 g ai/ha or less, 1660 g ai/ha or less, 1640 g ai/ha or less, 1620 g ai/ha or less, 1600 g ai/ha or less, 1550 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 900 g ai/ha or less, 800 g ai/ha or less, 700 g ai/ha or less, 600 g ai/ha or less, 500 g ai/ha or less, 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, or 110 g ai/ha or less; or in an amount ranging from any of the minimum values described above to any of the maximum values described above, such as 100-1700 g ai/ha, 125-850 g ai/ha, 130-700 g ai/ha, 150-1000 g ai/ha, 160-475 g ai/ha, 170-1660 g ai/ha, 180-1500 g ai/ha, 190-1050 g ai/ha, 200-700 g ai/ha, 250-1500 g ai/ha, 325-1200 g ai/ha, 450-1500 g ai/ha, 500-900 g ai/ha, or 110-1000 g ai/ha.

### Imazethapyr

Compositions and methods of the present disclosure can include imazethapyr or an agriculturally acceptable salt or ester thereof. Imazethapyr, shown below, is an imidazolinone that controls many major annual and perennial grass and broadleaf weeds in most major crops. Imazethapyr, as well as methods of preparing imazethapyr, are known in the art. Its herbicidal activity is described in The Pesticide Manual, Seventeenth Edition, 2016.

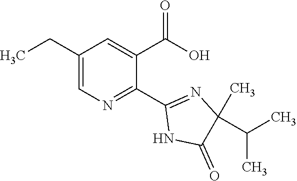 [SMILES: CCc1cnc(C2=NC(C)(C(C)C)C(=O)N2)c(C(=O)O)c1]

Imazethapyr can be applied to vegetation or an area adjacent the vegetation or applied to soil or water to prevent the emergence or growth of vegetation in an amount sufficient to induce a herbicidal effect. In some aspects, imazethapyr is applied to vegetation or an area adjacent the vegetation or applied to soil or water to prevent the emergence or growth of vegetation in an amount of 8.75 g ai/ha or more, such as 9 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 18 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 80 g ai/ha or more, 90 g ai/ha or more, 100 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 210 g ai/ha or more, 220 g ai/ha or more, 230 g ai/ha or more, 240 g ai/ha or more, 250 g ai/ha or more, 260 g ai/ha or more, 270 g ai/ha or more, 272 g ai/ha or more, 274 g ai/ha or more, 276 g ai/ha or more, or 278 g ai/ha or more; in an amount of 280 g ai/ha or less, such as 278 g ai/ha or less, 276 g ai/ha or less, 274 g ai/ha or less, 272 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 80 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 18 g ai/ha or less, 16 g ai/ha or less, 15 g ai/ha or less, 14 g ai/ha or less, 13 g ai/ha or less, 12 g ai/ha or less, 11 g ai/ha or less, 10 g ai/ha or less, or 9 g ai/ha or less; or in an amount ranging from any of the minimum values described above to any of the maximum values described above, such as 8.75-280 g ai/ha, 8.75-170 g ai/ha, 9-220 g ai/ha, 10-260 g ai/ha, 10-150 g ai/ha, 11-220 g ai/ha, 12-200 g ai/ha, 13-274 g ai/ha, 15-230 g ai/ha, 16-160 g ai/ha, 18-280 g ai/ha, 20-210 g ai/ha, 20-190 g ai/ha, or 25-272 g ai/ha.

### Pyrimidinyl Oxybenzoate Herbicides

In some aspects, the composition contains a herbicidally effective amount of compounds according to Formula I, and (b) bispyribac, pyribenzoxim, pyriftalid, pyriminobac, pyrimisulfan, pyrithiobac, or agriculturally acceptable salts or esters thereof, or combinations thereof.

### Bispyribac

Compositions and methods of the present disclosure can include bispyribac or an agriculturally acceptable salt or ester thereof. Bispyribac-sodium, shown below, is a pyrimidinyl oxybenzoate that provides control of grasses, sedges and broadleaf weeds, e.g., in direct-seeded and water-seeded rice and in turf. Bispyribac-sodium, as well as methods of preparing bispyribac-sodium, are known in the art. Its herbicidal activity is described in The Pesticide Manual, Seventeenth Edition, 2016.

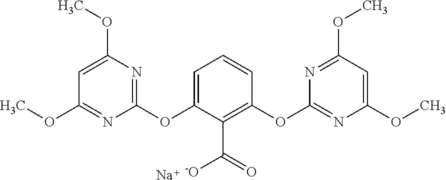

Bispyribac can be applied to vegetation or an area adjacent the vegetation or applied to soil or water to prevent the emergence or growth of vegetation in an amount sufficient to induce a herbicidal effect. In some aspects, bispyribac is applied to vegetation or an area adjacent the vegetation or applied to soil or water to prevent the emergence or growth of vegetation in an amount of 5 g ai/ha or more, such as 7.5 g ai/ha or more, 10 g ai/ha or more, 12.5 g ai/ha or more, 15 g ai/ha or more, 17.5 g ai/ha or more, 20 g ai/ha or more, 22.5 g ai/ha or more, 25 g ai/ha or more, 27.5 g ai/ha or more, 30 g ai/ha or more, 32.5 g ai/ha or more, 35 g ai/ha or more, 37.5 g ai/ha or more, 40 g ai/ha or more, 42.5 g ai/ha or more, 45 g ai/ha or more, 47.5 g ai/ha or more, 50 g ai/ha or more, 52.5 g ai/ha or more, 55 g ai/ha or more, 57.5 g ai/ha or more, 60 g ai/ha or more, 62.5 g ai/ha or more, 65 g ai/ha or more, 67.5 g ai/ha or more, 70 g ai/ha or more, 72.5 g ai/ha or more, 75 g ai/ha or more, 77.5 g ai/ha or more, 80 g ai/ha or more, 82.5 g ai/ha or more, 85 g ai/ha or more, 87.5 g ai/ha or more, 90 g ai/ha or more, 92.5 g ai/ha or more, 95 g ai/ha or more, 97.5 g ai/ha or more; in an amount of 100 g ai/ha or less, such as 97.5 g ai/ha or less, 95 g ai/ha or less, 92.5 g ai/ha or less, 90 g ai/ha or less, 87.5 g ai/ha or less, 85 g ai/ha or less, 82.5 g ai/ha or less, 80 g ai/ha or less, 77.5 g ai/ha or less, 75 g ai/ha or less, 72.5 g ai/ha or less, 70 g ai/ha or less, 67.5 g ai/ha or less, 65 g ai/ha or less, 62.5 g ai/ha or less, 60 g ai/ha or less, 57.5 g ai/ha or less, 55 g ai/ha or less, 52.5 g ai/ha or less, 50 g ai/ha or less, 47.5 g ai/ha or less, 45 g ai/ha or less, 42.5 g ai/ha or less, 40 g ai/ha or less, 37.5 g ai/ha or less, 35 g ai/ha or less, 32.5 g ai/ha or less, 30 g ai/ha or less, 27.5 g ai/ha or less, 25 g ai/ha or less, 22.5 g ai/ha or less, 20 g ai/ha or less, 17.5 g ai/ha or less, 15 g ai/ha or less, 12.5 g ai/ha or less, 10 g ai/ha or less, or 7.5 g ai/ha or less; or in an amount ranging from any of the minimum values described above to any of the maximum values described above, such as 5-100 g ai/ha, 5-90 g ai/ha, 7.5-45 g ai/ha, 10-55 g ai/ha, 15-90 g ai/ha, 20-50 g ai/ha, 22.5-90 g ai/ha, 25-52.5 g ai/ha, 25-80 g ai/ha, 25-65 g ai/ha, 27.5-60 g ai/ha, 27.5-47.5 g ai/ha, 30-85 g ai/ha, or 40-95 g ai/ha.

### Pyribenzoxim

Compositions and methods of the present disclosure can include pyribenzoxim or an agriculturally acceptable salt or ester thereof. Pyribenzoxim, shown below, is a pyrimidinyl oxybenzoate that provides post-emergence control of barnyard grass, blackgrass and Polygonum species in rice, wheat and zoysiagrass. Pyribenzoxim, as well as methods of preparing pyribenzoxim, are known in the art. Its herbicidal activity is described, for example, in The Pesticide Manual, Seventeenth Edition, 2016.

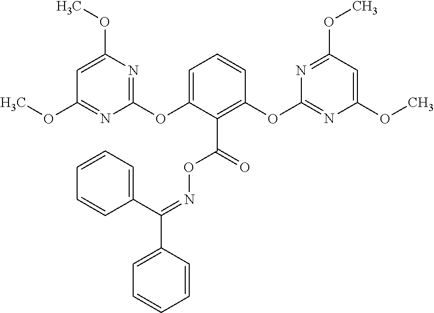

Pyribenzoxim can be applied to vegetation or an area adjacent the vegetation or applied to soil or water to prevent the emergence or growth of vegetation in an amount sufficient to induce a herbicidal effect. In some aspects, pyribenzoxim is applied to vegetation or an area adjacent the vegetation or applied to soil or water to prevent the emergence or growth of vegetation in an amount of 7.5 g ai/ha or more, such as 8 g ai/ha or more, 9 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 17 g ai/ha or more, 18 g ai/ha or more, 19 g ai/ha or more, 20 g ai/ha or more, 22 g ai/ha or more, 24 g ai/ha or more, 26 g ai/ha or more, 28 g ai/ha or more, 30 g ai/ha or more, 32 g ai/ha or more, 34 g ai/ha or more, 36 g ai/ha or more, 38 g ai/ha or more, 40 g ai/ha or more, 42 g ai/ha or more, 44 g ai/ha or more, 46 g ai/ha or more, 48 g ai/ha or more, 50 g ai/ha or more, 52 g ai/ha or more, 54 g ai/ha or more, 56 g ai/ha or more, 58 g ai/ha or more, 60 g ai/ha or more, 62 g ai/ha or more, 64 g ai/ha or more, 66 g ai/ha or more, 68 g ai/ha or more, or 69 g ai/ha or more; in an amount of 70 g ai/ha or less, such as 69 g ai/ha or less, 68 g ai/ha or less, 66 g ai/ha or less, 64 g ai/ha or less, 62 g ai/ha or less, 60 g ai/ha or less, 58 g ai/ha or less, 56 g ai/ha or less, 54 g ai/ha or less, 52 g ai/ha or less, 50 g ai/ha or less, 48 g ai/ha or less, 46 g ai/ha or less, 44 g ai/ha or less, 42 g ai/ha or less, 40 g ai/ha or less, 38 g ai/ha or less, 36 g ai/ha or less, 34 g ai/ha or less, 32 g ai/ha or less, 30 g ai/ha or less, 28 g ai/ha or less, 26 g ai/ha or less, 24 g ai/ha or less, 22 g ai/ha or less, 20 g ai/ha or less, 19 g ai/ha or less, 18 g ai/ha or less, 17 g ai/ha or less, 16 g ai/ha or less, 15 g ai/ha or less, 14 g ai/ha or less, 13 g ai/ha or less, 12 g ai/ha or less, 11 g ai/ha or less, 10 g ai/ha or less, 9 g ai/ha or less, or 8 g ai/ha or less; or in an amount ranging from any of the minimum values described above to any of the maximum values described above, such as 7.5-70 g ai/ha, 7.5-68 g ai/ha, 7.5-52 g ai/ha, 8-60 g ai/ha, 9-38 g ai/ha, 9-64 g ai/ha, 10-66 g ai/ha, 10-44 g ai/ha, 10-40 g ai/ha, 11-62 g ai/ha, 12-46 g ai/ha, 13-70 g ai/ha, 15-54 g ai/ha, or 20-64 g ai/ha.

### Pyriftalid

Compositions and methods of the present disclosure can include pyriftalid or an agriculturally acceptable salt or ester thereof. Pyriftalid, shown below, is a pyrimidinyl oxybenzoate that provides control of grass weeds in rice. Pyriftalid, as well as methods of

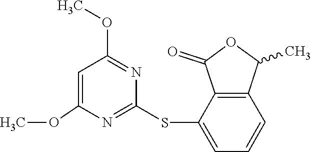

preparing pyriftalid, are known in the art. Its herbicidal activity is described, for example, in The Pesticide Manual, Seventeenth Edition, 2016.

Pyriftalid can be applied to vegetation or an area adjacent the vegetation or applied to soil or water to prevent the emergence or growth of vegetation in an amount sufficient to induce a herbicidal effect. In some aspects, pyriftalid is applied to vegetation or an area adjacent the vegetation or applied to soil or water to prevent the emergence or growth of vegetation in an amount of 20 g ai/ha or more, such as 22 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 70 g ai/ha or more, 80 g ai/ha or more, 90 g ai/ha or more, 100 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 210 g ai/ha or more, 220 g ai/ha or more, 230 g ai/ha or more, 240 g ai/ha or more, 250 g ai/ha or more, 260 g ai/ha or more, 270 g ai/ha or more, 280 g ai/ha or more, 290 g ai/ha or more, 300 g ai/ha or more, 310 g ai/ha or more, 320 g ai/ha or more, 330 g ai/ha or more, 340 g ai/ha or more, or 345 g ai/ha or more; in an amount of 350 g ai/ha or less, such as 345 g ai/ha or less, 340 g ai/ha or less, 330 g ai/ha or less, 320 g ai/ha or less, 310 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 80 g ai/ha or less, 70 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, or 22 g ai/ha or less; or in an amount ranging from any of the minimum values described above to any of the maximum values described above, such as 20-350 g ai/ha, 55-170 g ai/ha, 22-220 g ai/ha, 70-260 g ai/ha, 30-250 g ai/ha, 45-220 g ai/ha, 80-200 g ai/ha, 130-270 g ai/ha, 150-330 g ai/ha, 60-160 g ai/ha, 180-345 g ai/ha, 210-310 g ai/ha, 90-190 g ai/ha, or 22-340 g ai/ha.

### Pyriminobac

Compositions and methods of the present disclosure can include pyriminobac or an agriculturally acceptable salt or ester thereof. Pyriminobac, shown below, is a pyrimidinyl oxybenzoate that provides post-emergence control of Echinochloa spp., in paddy rice. Pyriminobac, as well as methods of preparing pyriminobac, are known in the art. Its herbicidal activity is described, for example, in The Pesticide Manual, Seventeenth Edition, 2016.

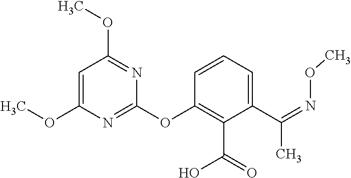

Pyriminobac can be applied to vegetation or an area adjacent the vegetation or applied to soil or water to prevent the emergence or growth of vegetation in an amount sufficient to induce a herbicidal effect. In some aspects, pyriminobac is applied to vegetation or an area adjacent the vegetation or applied to soil or water to prevent the emergence or growth of vegetation in an amount of 20 g ai/ha or more, such as 22 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 80 g ai/ha or more, 90 g ai/ha or more, 100 g ai/ha or more, 120 g ai/ha or more, 130 g ai/ha or more, 140 g ai/ha or more, 150 g ai/ha or more, 160 g ai/ha or more, or 170 g ai/ha or more; in an amount of 175 g ai/ha or less, such as 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 80 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, or 22 g ai/ha or less; or in an amount ranging from any of the minimum values described above to any of the maximum values described above, such as 20-175 g ai/ha, 22-170 g ai/ha, 35-120 g ai/ha, 100-160 g ai/ha, 40-150 g ai/ha, 80-120 g ai/ha, 25-110 g ai/ha, 90-150 g ai/ha, 45-130 g ai/ha, 60-160 g ai/ha, 30-120 g ai/ha, 20-160 g ai/ha, or 22-100 g ai/ha.

### Sulfonylaminocarbonyl Triazolinone Herbicides

In some aspects, the composition contains a herbicidally effective amount of compounds according to Formula I, and (b) flucarbazone, propoxycarbazone, thiencarbazone, or agriculturally acceptable salts or esters thereof, or combinations thereof.

### Flucarbazone

Compositions and methods of the present disclosure can include flucarbazone or an agriculturally acceptable salt or ester thereof. Flucarbazone, shown below, is a sulfonylamino-carbonyl triazolinone that provides post-emergence control of annual grass weeds and some perennial grass weeds and some broad-leaved weeds. Flucarbazone, as well as methods of preparing flucarbazone, are known in the art. Its herbicidal activity is described in The Pesticide Manual, Seventeenth Edition, 2016.

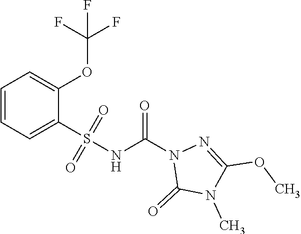 [SMILES: COc1nn(C(=O)NS(=O)(=O)c2ccccc2OC(F)(F)F)c(=O)n1C]

Flucarbazone can be applied to vegetation or an area adjacent the vegetation or applied to soil or water to prevent the emergence or growth of vegetation in an amount sufficient to induce a herbicidal effect. In some aspects, flucarbazone is applied to vegetation or an area adjacent the vegetation or applied to soil or water to prevent the emergence or growth of vegetation in an amount of 3.75 g ai/ha or more, such as 4 g ai/ha or more, 4.25 g ai/ha or more, 4.5 g ai/ha or more, 4.75 g ai/ha or more, 5 g ai/ha or more, 5.25 g ai/ha or more, 5.5 g ai/ha or more, 5.75 g ai/ha or more, 6 g ai/ha or more, 7 g ai/ha or more, 7.5 g ai/ha or more, 8 g ai/ha or more, 9 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 17 g ai/ha or more, 18 g ai/ha or more, 19 g ai/ha or more, 20 g ai/ha or more, 21 g ai/ha or more, 22 g ai/ha or more, 23 g ai/ha or more, 24 g ai/ha or more, 25 g ai/ha or more, 25.5 g ai/ha or more, 26 g ai/ha or more, 26.5 g ai/ha or more, 27 g ai/ha or more, 27.5 g ai/ha or more, 28 g ai/ha or more, 28.5 g ai/ha or more, 29 g ai/ha or more, 29.25 g ai/ha or more, 29.5 g ai/ha or more, or 29.75 g ai/ha or more; in an amount of 30 g ai/ha or less, such as 29.75 g ai/ha or less, 29.5 g ai/ha or less, 29.25 g ai/ha or less, 29 g ai/ha or less, 28.5 g ai/ha or less, 28 g ai/ha or less, 27.5 g ai/ha or less, 27 g ai/ha or less, 26.5 g ai/ha or less, 26 g ai/ha or less, 25 g ai/ha or less, 24 g ai/ha or less, 23 g ai/ha or less, 22 g ai/ha or less, 21 g ai/ha or less, 20 g ai/ha or less, 19 g ai/ha or less, 18 g ai/ha or less, 17 g ai/ha or less, 16 g ai/ha or less, 15 g ai/ha or less, 14 g ai/ha or less, 13 g ai/ha or less, 12 g ai/ha or less, 11 g ai/ha or less, 10 g ai/ha or less, 9 g ai/ha or less, 8 g ai/ha or less, 7.5 g ai/ha or less, 7 g ai/ha or less, 6 g ai/ha or less, 5.75 g ai/ha or less, 5.5 g ai/ha or less, 5.25 g ai/ha or less, 5 g ai/ha or less, 4.75 g ai/ha or less, 4.5 g ai/ha or less, 4.25 g ai/ha or less, or 4 g ai/ha or less; or in an amount ranging from any of the minimum values described above to any of the maximum values described above, such as 3.75-30 g ai/ha, 3.75-27 g ai/ha, 4-28 g ai/ha, 4-20 g ai/ha, 5-29.5 g ai/ha, 5-21 g ai/ha, 5.5-28 g ai/ha, 6-26 g ai/ha, 7-25 g ai/ha, 7.5-30 g ai/ha, 7.5-21 g ai/ha, 8-25 g ai/ha, 9-19 g ai/ha, or 10-28.5 g ai/ha.

### Propoxycarbazone

Compositions and methods of the present disclosure can include propoxycarbazone or an agriculturally acceptable salt or ester thereof. Propoxycarbazone, shown below, is a sulfonylaminocarbonyl triazolinone that provides post-emergence control of grass weeds and some broad-leaved weeds. Propoxycarbazone, as well as methods of preparing propoxycarbazone, are known in the art. Its herbicidal activity is described in The Pesticide Manual, Seventeenth Edition, 2016.

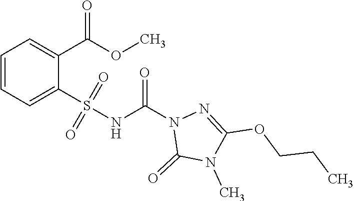 [SMILES: CCCOc1nn(C(=O)NS(=O)(=O)c2ccccc2C(=O)OC)c(=O)n1C]

Propoxycarbazone can be applied to vegetation or an area adjacent the vegetation or applied to soil or water to prevent the emergence or growth of vegetation in an amount sufficient to induce a herbicidal effect. In some aspects, propoxycarbazone is applied to vegetation or an area adjacent the vegetation or applied to soil or water to prevent the emergence or growth of vegetation in an amount of 3.75 g ai/ha or more, such as 4 g ai/ha or more, 4.25 g ai/ha or more, 4.5 g ai/ha or more, 4.75 g ai/ha or more, 5 g ai/ha or more, 6 g ai/ha or more, 7 g ai/ha or more, 8 g ai/ha or more, 9 g ai/ha or more, 10 g ai/ha or more, 12 g ai/ha or more, 14 g ai/ha or more, 16 g ai/ha or more, 18 g ai/ha or more, 20 g ai/ha or more, 22 g ai/ha or more, 24 g ai/ha or more, 26 g ai/ha or more, 28 g ai/ha or more, 30 g ai/ha or more, 32 g ai/ha or more, 34 g ai/ha or more, 36 g ai/ha or more, 38 g ai/ha or more, 40 g ai/ha or more, 42 g ai/ha or more, 44 g ai/ha or more, 46 g ai/ha or more, 48 g ai/ha or more, 50 g ai/ha or more, 52 g ai/ha or more, 54 g ai/ha or more, 56 g ai/ha or more, 58 g ai/ha or more, 60 g ai/ha or more, 61 g ai/ha or more, 62 g ai/ha or more, 63 g ai/ha or more, 64 g ai/ha or more, 65 g ai/ha or more, 66 g ai/ha or more, 67 g ai/ha or more, 68 g ai/ha or more, or 69 g ai/ha or more; in an amount of 70 g ai/ha or less, such as 69 g ai/ha or less, 68 g ai/ha or less, 67 g ai/ha or less, 66 g ai/ha or less, 65 g ai/ha or less, 64 g ai/ha or less, 63 g ai/ha or less, 62 g ai/ha or less, 61 g ai/ha or less, 60 g ai/ha or less, 58 g ai/ha or less, 56 g ai/ha or less, 54 g ai/ha or less, 52 g ai/ha or less, 50 g ai/ha or less, 48 g ai/ha or less, 46 g ai/ha or less, 44 g ai/ha or less, 42 g ai/ha or less, 40 g ai/ha or less, 38 g ai/ha or less, 36 g ai/ha or less, 34 g ai/ha or less, 32 g ai/ha or less, 30 g ai/ha or less, 28 g ai/ha or less, 26 g ai/ha or less, 24 g ai/ha or less, 22 g ai/ha or less, 20 g ai/ha or less, 28 g ai/ha or less, 16 g ai/ha or less, 14 g ai/ha or less, 12 g ai/ha or less, 10 g ai/ha or less, 9 g ai/ha or less, 8 g ai/ha or less, 7 g ai/ha or less, 6 g ai/ha or less, 5 g ai/ha or less, 4.75 g ai/ha or less, 4.5 g ai/ha or less, 4.25 g ai/ha or less, or 4 g ai/ha or less; or in an amount ranging from any of the minimum values described above to any of the maximum values described above, such as 3.75-70 g ai/ha, 4-68 g ai/ha, 4.25-56 g ai/ha, 4.5-28 g ai/ha, 5-62 g ai/ha, 5-38 g ai/ha, 7-70 g ai/ha, 8-52 g ai/ha, 9-40 g ai/ha, 10-70 g ai/ha, 10-24 g ai/ha, 12-69 g ai/ha, 14-65 g ai/ha, or 20-58 g ai/ha.

### Thiencarbazone

Compositions and methods of the present disclosure can include thiencarbazone or an agriculturally acceptable salt or ester thereof. Thiencarbazone-methyl, shown below, is a sulfonylaminocarbonyl triazolinone that provides post-emergence control of grass weeds and some broad-leaved weeds. Thiencarbazone, as well as methods of preparing thiencarbazone, are known in the art. Its herbicidal activity is described in The Pesticide Manual, Seventeenth Edition, 2016.

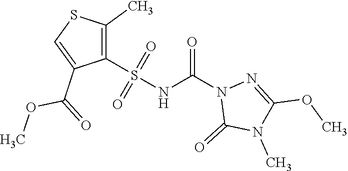

Thiencarbazone can be applied to vegetation or an area adjacent the vegetation or applied to soil or water to prevent the emergence or growth of vegetation in an amount sufficient to induce a herbicidal effect. In some aspects, thiencarbazone is applied to vegetation or an area adjacent the vegetation or applied to soil or water to prevent the emergence or growth of vegetation in an amount of 2.5 g ai/ha or more, such as 2.75 g ai/ha or more, 3 g ai/ha or more, 3.25 g ai/ha or more, 3.5 g ai/ha or more, 3.75 g ai/ha or more, 4 g ai/ha or more, 4.25 g ai/ha or more, 4.5 g ai/ha or more, 4.75 g ai/ha or more, 5 g ai/ha or more, 6 g ai/ha or more, 7 g ai/ha or more, 8 g ai/ha or more, 9 g ai/ha or more, 10 g ai/ha or more, 12 g ai/ha or more, 14 g ai/ha or more, 16 g ai/ha or more, 18 g ai/ha or more, 20 g ai/ha or more, 22 g ai/ha or more, 24 g ai/ha or more, 26 g ai/ha or more, 28 g ai/ha or more, 30 g ai/ha or more, 31 g ai/ha or more, 32 g ai/ha or more, 33 g ai/ha or more, 34 g ai/ha or more, 35 g ai/ha or more, 36 g ai/ha or more, 37 g ai/ha or more, 38 g ai/ha or more, 39 g ai/ha or more, 40 g ai/ha or more, 41 g ai/ha or more, 42 g ai/ha or more, 43 g ai/ha or more, or 44 g ai/ha or more; in an amount of 45 g ai/ha or less, such as 44 g ai/ha or less, 43 g ai/ha or less, 42 g ai/ha or less, 41 g ai/ha or less, 40 g ai/ha or less, 39 g ai/ha or less, 38 g ai/ha or less, 37 g ai/ha or less, 36 g ai/ha or less, 35 g ai/ha or less, 34 g ai/ha or less, 33 g ai/ha or less, 32 g ai/ha or less, 31 g ai/ha or less, 30 g ai/ha or less, 28 g ai/ha or less, 26 g ai/ha or less, 24 g ai/ha or less, 22 g ai/ha or less, 20 g ai/ha or less, 18 g ai/ha or less, 16 g ai/ha or less, 14 g ai/ha or less, 12 g ai/ha or less, 10 g ai/ha or less, 9 g ai/ha or less, 8 g ai/ha or less, 7 g ai/ha or less, 6 g ai/ha or less, 5 g ai/ha or less, 4.75 g ai/ha or less, 4.5 g ai/ha or less, 4.25 g ai/ha or less, 4 g ai/ha or less, 3.75 g ai/ha or less, 3.5 g ai/ha or less, 3.25 g ai/ha or less, 3 g ai/ha or less, or 2.75 g ai/ha or less; or in an amount ranging from any of the minimum values described above to any of the maximum values described above, such as 2.5-45 g ai/ha, 2.5-41 g ai/ha, 2.75-39 g ai/ha, 2.75-37 g ai/ha, 3-40 g ai/ha, 3-33 g ai/ha, 3.25-28 g ai/ha, 3.5-45 g ai/ha, 3.75-45 g ai/ha, 4-39 g ai/ha, 4.5-31 g ai/ha, 5-45 g ai/ha, 5-37 g ai/ha, or 8-36 g ai/ha.

### Sulfonylurea Herbicides

In some aspects, the composition contains a herbicidally effective amount of compounds according to Formula I, and (b) amidosulfuron, azimsulfuron, bensulfuron, chlorimuron, chlorsulfuron, cinosulfuron, cyclosulfamuron, ethametsulfuron, ethoxysulfuron, flazasulfuron, flucetosulfuron, flupyrsulfuron, foramsulfuron, halosulfuron, imazosulfuron, iodosulfuron, iofensulfuron, mesosulfuron, metazosulfuron, metsulfuron, nicosulfuron, orthosulfamuron, oxasulfuron, primisulfuron, propyrisulfuron, prosulfuron, pyrazosulfuron, rimsulfuron, sulfometuron, sulfosulfuron, triasulfuron, tribenuron, thifensulfuron, trifloxysulfuron, triflusulfuron, tritosulfuron, or agriculturally acceptable salts or esters thereof, or combinations thereof.

### Amidosulfuron

Compositions and methods of the present disclosure can include amidosulfuron or an agriculturally acceptable salt or ester thereof. Amidosulfuron, shown below, is a sulfonylurea that provides post-emergence control of a wide range of broadleaf weeds, e.g., cleavers, in winter wheat, durum wheat, barley, rye, triticale and oats. Amidosulfuron, as well as methods of

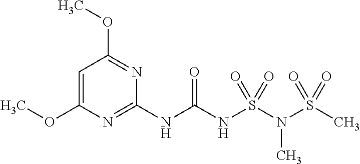

preparing amidosulfuron, are known in the art. Its herbicidal activity is described in The Pesticide Manual, Seventeenth Edition, 2016.

Amidosulfuron can be applied to vegetation or an area adjacent the vegetation or applied to soil or water to prevent the emergence or growth of vegetation in an amount sufficient to induce a herbicidal effect. In some aspects, amidosulfuron is applied to vegetation or an area adjacent the vegetation or applied to soil or water to prevent the emergence or growth of vegetation in an amount of 3.75 g ai/ha or more, such as 4 g ai/ha or more, 4.25 g ai/ha or more, 4.5 g ai/ha or more, 4.75 g ai/ha or more, 5 g ai/ha or more, 6 g ai/ha or more, 7 g ai/ha or more, 8 g ai/ha or more, 9 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 17 g ai/ha or more, 18 g ai/ha or more, 19 g ai/ha or more, 20 g ai/ha or more, 22 g ai/ha or more, 24 g ai/ha or more, 26 g ai/ha or more, 28 g ai/ha or more, 30 g ai/ha or more, 32 g ai/ha or more, 34 g ai/ha or more, 36 g ai/ha or more, 38 g ai/ha or more, 40 g ai/ha or more, 42 g ai/ha or more, 44 g ai/ha or more, 46 g ai/ha or more, 48 g ai/ha or more, 50 g ai/ha or more, 51 g ai/ha or more, 52 g ai/ha or more, 53 g ai/ha or more, 54 g ai/ha or more, 55 g ai/ha or more, 56 g ai/ha or more, 57 g ai/ha or more, 58 g ai/ha or more, or 59 g ai/ha or more; in an amount of 60 g ai/ha or less, such as 59 g ai/ha or less, 58 g ai/ha or less, 57 g ai/ha or less, 56 g ai/ha or less, 55 g ai/ha or less, 54 g ai/ha or less, 53 g ai/ha or less, 52 g ai/ha or less, 51 g ai/ha or less, 50 g ai/ha or less, 48 g ai/ha or less, 46 g ai/ha or less, 44 g ai/ha or less, 42 g ai/ha or less, 40 g ai/ha or less, 38 g ai/ha or less, 36 g ai/ha or less, 34 g ai/ha or less, 32 g ai/ha or less, 30 g ai/ha or less, 28 g ai/ha or less, 26 g ai/ha or less, 24 g ai/ha or less, 22 g ai/ha or less, 20 g ai/ha or less, 19 g ai/ha or less, 18 g ai/ha or less, 17 g ai/ha or less, 16 g ai/ha or less, 15 g ai/ha or less, 14 g ai/ha or less, 13 g ai/ha or less, 12 g ai/ha or less, 11 g ai/ha or less, 10 g ai/ha or less, 9 g ai/ha or less, 8 g ai/ha or less, 7 g ai/ha or less, 6 g ai/ha or less, 5 g ai/ha or less, 4.75 g ai/ha or less, 4.5 g ai/ha or less, or 4.25 g ai/ha or less, or 4 g ai/ha or less; or in an amount ranging from any of the minimum values described above to any of the maximum values described above, such as 3.75-60 g ai/ha, 4-58 g ai/ha, 4.5-51 g ai/ha, 5-57 g ai/ha, 5-30 g ai/ha, 6-42 g ai/ha, 8-50 g ai/ha, 9-32 g ai/ha, 10-53 g ai/ha, 12-30 g ai/ha, or 13-40 g ai/ha.

### Azimsulfuron

Compositions and methods of the present disclosure can include azimsulfuron or an agriculturally acceptable salt or ester thereof. Azimsulfuron, shown below, is a sulfonylurea that provides e.g., post-emergence control of annual and perennial broadleaf and sedge weeds in rice. Azimsulfuron, as well as methods of preparing azimsulfuron, are known in the art. Its herbicidal activity is described in The Pesticide Manual, Seventeenth Edition, 2016.

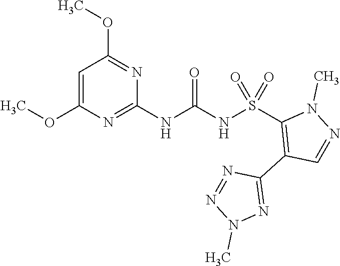 [SMILES: COc1cc(OC)nc(NC(=O)NS(=O)(=O)c2c(-c3nnn(C)n3)cnn2C)n1]

Azimsulfuron can be applied to vegetation or an area adjacent the vegetation or applied to soil or water to prevent the emergence or growth of vegetation in an amount sufficient to induce a herbicidal effect. In some aspects, azimsulfuron is applied to vegetation or an area adjacent the vegetation or applied to soil or water to prevent the emergence or growth of vegetation in an amount of 3.125 g ai/ha or more, such as 3.25 g ai/ha or more, 3.5 g ai/ha or more, 3.75 g ai/ha or more, 4 g ai/ha or more, 4.25 g ai/ha or more, 4.5 g ai/ha or more, 4.75 g ai/ha or more, 5 g ai/ha or more, 5.5 g ai/ha or more, 6 g ai/ha or more, 6.5 g ai/ha or more, 7 g ai/ha or more, 7.5 g ai/ha or more, 8 g ai/ha or more, 8.5 g ai/ha or more, 9 g ai/ha or more, 9.5 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 17 g ai/ha or more, 18 g ai/ha or more, 19 g ai/ha or more, 20 g ai/ha or more, 20.5 g ai/ha or more, 21 g ai/ha or more, 21.5 g ai/ha or more, 22 g ai/ha or more, 22.5 g ai/ha or more, 22.75 g ai/ha or more, 23 g ai/ha or more, 23.25 g ai/ha or more, 23.5 g ai/ha or more, 23.75 g ai/ha or more, 24 g ai/ha or more, 24.25 g ai/ha or more, 24.5 g ai/ha or more, or 24.75 g ai/ha or more; in an amount of 25 g ai/ha or less, such as 24.75 g ai/ha or less, 24.5 g ai/ha or less, 24.25 g ai/ha or less, 24 g ai/ha or less, 23.75 g ai/ha or less, 23.5 g ai/ha or less, 23.25 g ai/ha or less, 23 g ai/ha or less, 22.75 g ai/ha or less, 22.5 g ai/ha or less, 22 g ai/ha or less, 21.5 g ai/ha or less, 21 g ai/ha or less, 20.5 g ai/ha or less, 20 g ai/ha or less, 19 g ai/ha or less, 18 g ai/ha or less, 17 g ai/ha or less, 16 g ai/ha or less, 15 g ai/ha or less, 14 g ai/ha or less, 13 g ai/ha or less, 12 g ai/ha or less, 11 g ai/ha or less, 10 g ai/ha or less, 9.5 g ai/ha or less, 9 g ai/ha or less, 8.5 g ai/ha or less, 8 g ai/ha or less, 7.5 g ai/ha or less, 7 g ai/ha or less, 6.5 g ai/ha or less, 6 g ai/ha or less, 5.5 g ai/ha or less, 5 g ai/ha or less, 4.75 g ai/ha or less, 4.5 g ai/ha or less, 4.25 g ai/ha or less, 4 g ai/ha or less, 3.75 g ai/ha or less, 3.5 g ai/ha or less, or 3.25 g ai/ha or less; or in an amount ranging from any of the minimum values described above to any of the maximum values described above, such as 3.125-25 g ai/ha, 3.125-23.5 g ai/ha, 3.125-19 g ai/ha, 3.25-22 g ai/ha, 3.5-24.5 g ai/ha, 3.5-19 g ai/ha, 3.75-22 g ai/ha, 4-25 g ai/ha, 4-23.25 g ai/ha, 4.25-20 g ai/ha, 4.5-18 g ai/ha, 4.75-22.75 g ai/ha, or 5-24 g ai/ha.

### Bensulfuron

Compositions and methods of the present disclosure can include bensulfuron or an agriculturally acceptable salt or ester thereof. Bensulfuron-methyl, shown below, is a sulfonylurea that provides e.g., pre- and post-emergence control of annual and perennial broadleaf weeds and sedges in rice. Bensulfuron, as well as methods of preparing bensulfuron, are known in the art. Its herbicidal activity is described in The Pesticide Manual, Seventeenth Edition, 2016.

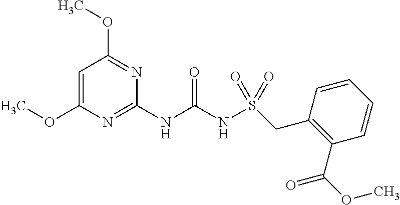

Bensulfuron can be applied to vegetation or an area adjacent the vegetation or applied to soil or water to prevent the emergence or growth of vegetation in an amount sufficient to induce a herbicidal effect. In some aspects, bensulfuron is applied to vegetation or an area adjacent the vegetation or applied to soil or water to prevent the emergence or growth of vegetation in an amount of 8.75 g ai/ha or more, such as 9 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 17 g ai/ha or more, 18 g ai/ha or more, 19 g ai/ha or more, 20 g ai/ha or more, 22 g ai/ha or more, 24 g ai/ha or more, 26 g ai/ha or more, 28 g ai/ha or more, 30 g ai/ha or more, 32 g ai/ha or more, 34 g ai/ha or more, 36 g ai/ha or more, 38 g ai/ha or more, 40 g ai/ha or more, 42 g ai/ha or more, 44 g ai/ha or more, 46 g ai/ha or more, 48 g ai/ha or more, 50 g ai/ha or more, 52 g ai/ha or more, 54 g ai/ha or more, 56 g ai/ha or more, 58 g ai/ha or more, 60 g ai/ha or more, 62 g ai/ha or more, 64 g ai/ha or more, 66 g ai/ha or more, 68 g ai/ha or more, or 69 g ai/ha or more; in an amount of 70 g ai/ha or less, such as 69 g ai/ha or less, 68 g ai/ha or less, 66 g ai/ha or less, 64 g ai/ha or less, 62 g ai/ha or less, 60 g ai/ha or less, 58 g ai/ha or less, 56 g ai/ha or less, 54 g ai/ha or less, 52 g ai/ha or less, 50 g ai/ha or less, 48 g ai/ha or less, 46 g ai/ha or less, 44 g ai/ha or less, 42 g ai/ha or less, 40 g ai/ha or less, 38 g ai/ha or less, 36 g ai/ha or less, 34 g ai/ha or less, 32 g ai/ha or less, 30 g ai/ha or less, 28 g ai/ha or less, 26 g ai/ha or less, 24 g ai/ha or less, 22 g ai/ha or less, 20 g ai/ha or less, 19 g ai/ha or less, 18 g ai/ha or less, 17 g ai/ha or less, 16 g ai/ha or less, 15 g ai/ha or less, 14 g ai/ha or less, 13 g ai/ha or less, 12 g ai/ha or less, 11 g ai/ha or less, 10 g ai/ha or less, or 9 g ai/ha or less; or in an amount ranging from any of the minimum values described above to any of the maximum values described above, such as 8.75-70 g ai/ha, 9-60 g ai/ha, 10-65 g ai/ha, 10-40 g ai/ha, 10-28 g ai/ha, 11-68 g ai/ha, 11-54 g ai/ha, 12-60 g ai/ha, 12-42 g ai/ha, 13-69 g ai/ha, 14-56 g ai/ha, 15-40 g ai/ha, 20-46 g ai/ha, or 20-69 g ai/ha.

### Chlorsulfuron

Compositions and methods of the present disclosure can include chlorsulfuron or an agriculturally acceptable salt or ester thereof. Chlorsulfuron, shown below, is a sulfonylurea that provides control of e.g., broadleaf weeds and annual grasses in wheat, barley, oats, rye, triticale, flax and on non-crop land. Chlorsulfuron, as well as methods of preparing chlorsulfuron, are known in the art. Its herbicidal activity is described in The Pesticide Manual, Seventeenth Edition, 2016.

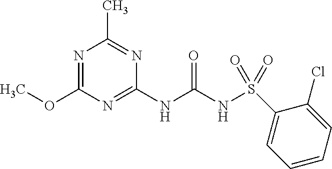 [SMILES: COc1nc(C)nc(NC(=O)NS(=O)(=O)c2ccccc2Cl)n1]

Chlorsulfuron can be applied to vegetation or an area adjacent the vegetation or applied to soil or water to prevent the emergence or growth of vegetation in an amount sufficient to induce a herbicidal effect. In some aspects, chlorsulfuron is applied to vegetation or an area adjacent the vegetation or applied to soil or water to prevent the emergence or growth of vegetation in an amount of 1.1 g ai/ha or more, such as 1.25 g ai/ha or more, 1.5 g ai/ha or more, 1.75 g ai/ha or more, 2 g ai/ha or more, 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.25 g ai/ha or more, 4.5 g ai/ha or more, 4.75 g ai/ha or more, 5 g ai/ha or more, 6 g ai/ha or more, 7 g ai/ha or more, 8 g ai/ha or more, 9 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 17 g ai/ha or more, 18 g ai/ha or more, 19 g ai/ha or more, 20 g ai/ha or more, 22 g ai/ha or more, 24 g ai/ha or more, 26 g ai/ha or more, 28 g ai/ha or more, 30 g ai/ha or more, 32 g ai/ha or more, 34 g ai/ha or more, 36 g ai/ha or more, 38 g ai/ha or more, 40 g ai/ha or more, 42 g ai/ha or more, 44 g ai/ha or more, 46 g ai/ha or more, 47 g ai/ha or more, 48 g ai/ha or more, 49 g ai/ha or more, 50 g ai/ha or more, 51 g ai/ha or more, or 51.5 g ai/ha or more; in an amount of 52 g ai/ha or less, such as 51.5 g ai/ha or less, 51 g ai/ha or less, 50 g ai/ha or less, 49 g ai/ha or less, 48 g ai/ha or less, 47 g ai/ha or less, 46 g ai/ha or less, 44 g ai/ha or less, 42 g ai/ha or less, 40 g ai/ha or less, 38 g ai/ha or less, 36 g ai/ha or less, 34 g ai/ha or less, 32 g ai/ha or less, 30 g ai/ha or less, 28 g ai/ha or less, 26 g ai/ha or less, 24 g ai/ha or less, 22 g ai/ha or less, 20 g ai/ha or less, 19 g ai/ha or less, 18 g ai/ha or less, 17 g ai/ha or less, 16 g ai/ha or less, 15 g ai/ha or less, 14 g ai/ha or less, 13 g ai/ha or less, 12 g ai/ha or less, 11 g ai/ha or less, 10 g ai/ha or less, 9 g ai/ha or less, 8 g ai/ha or less, 7 g ai/ha or less, 6 g ai/ha or less, 5 g ai/ha or less, 4.75 g ai/ha or less, 4.5 g ai/ha or less, or 4.25 g ai/ha or less, ai/ha or less, 4 g ai/ha or less, 3.75 g ai/ha or less, 3.5 g ai/ha or less, 3.25 g ai/ha or less, 3 g ai/ha or less, 2.75 g ai/ha or less, 2.5 g ai/ha or less, 2.25 g ai/ha or less, 2 g ai/ha or less, 1.75 g ai/ha or less, 1.5 g ai/ha or less, or 1.25 g ai/ha or less; or in an amount ranging from any of the minimum values described above to any of the maximum values described above, such as 1.1-52 g ai/ha, 1.25-51.5 g ai/ha, 1.5-30 g ai/ha, 1.75-47 g ai/ha, 2-26 g ai/ha, 3-50 g ai/ha, 4-38 g ai/ha, 4.5-42 g ai/ha, 5-51 g ai/ha, 7-49 g ai/ha, 9-32 g ai/ha, 10-45 g ai/ha, 12-52 g ai/ha, or 15-48 g ai/ha.

### Ethoxysulfuron

Compositions and methods of the present disclosure can include ethoxysulfuron or an agriculturally acceptable salt or ester thereof. Ethoxysulfuron, shown below, is a sulfonylurea that provides e.g., control of broadleaf and sedge weeds in cereals, rice and sugar cane. Ethoxysulfuron, as well as methods of preparing ethoxysulfuron, are known in the art. Its herbicidal activity is described in The Pesticide Manual, Seventeenth Edition, 2016.

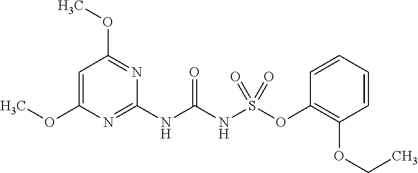 [SMILES: CCOc1ccccc1OS(=O)(=O)NC(=O)Nc1nc(OC)cc(OC)n1]

Ethoxysulfuron can be applied to vegetation or an area adjacent the vegetation or applied to soil or water to prevent the emergence or growth of vegetation in an amount sufficient to induce a herbicidal effect. In some aspects, ethoxysulfuron is applied to vegetation or an area adjacent the vegetation or applied to soil or water to prevent the emergence or growth of vegetation in an amount of 5 g ai/ha or more, such as 6 g ai/ha or more, 7 g ai/ha or more, 8 g ai/ha or more, 9 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 17 g ai/ha or more, 18 g ai/ha or more, 19 g ai/ha or more, 20 g ai/ha or more, 22 g ai/ha or more, 24 g ai/ha or more, 26 g ai/ha or more, 28 g ai/ha or more, 30 g ai/ha or more, 32 g ai/ha or more, 34 g ai/ha or more, 36 g ai/ha or more, 38 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 101 g ai/ha or more, 102 g ai/ha or more, 103 g ai/ha or more, 104 g ai/ha or more, 105 g ai/ha or more, 106 g ai/ha or more, 107 g ai/ha or more, 108 g ai/ha or more, 109 g ai/ha or more, 110 g ai/ha or more, 111 g ai/ha or more, 112 g ai/ha or more, 113 g ai/ha or more, 114 g ai/ha or more, 115 g ai/ha or more, 116 g ai/ha or more, 117 g ai/ha or more, 118 g ai/ha or more, or 119 g ai/ha or more; in an amount of 120 g ai/ha or less, such as 119 g ai/ha or less, 118 g ai/ha or less, 117 g ai/ha or less, 116 g ai/ha or less, 115 g ai/ha or less, 114 g ai/ha or less, 113 g ai/ha or less, 112 g ai/ha or less, 111 g ai/ha or less, 110 g ai/ha or less, 109 g ai/ha or less, 108 g ai/ha or less, 107 g ai/ha or less, 106 g ai/ha or less, 105 g ai/ha or less, 104 g ai/ha or less, 103 g ai/ha or less, 102 g ai/ha or less, 101 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 38 g ai/ha or less, 36 g ai/ha or less, 34 g ai/ha or less, 32 g ai/ha or less, 30 g ai/ha or less, 28 g ai/ha or less, 26 g ai/ha or less, 24 g ai/ha or less, 22 g ai/ha or less, 20 g ai/ha or less, 19 g ai/ha or less, 18 g ai/ha or less, 17 g ai/ha or less, 16 g ai/ha or less, 15 g ai/ha or less, 14 g ai/ha or less, 13 g ai/ha or less, 12 g ai/ha or less, 11 g ai/ha or less, 10 g ai/ha or less, 9 g ai/ha or less, 8 g ai/ha or less, 7 g ai/ha or less, or 6 g ai/ha or less; or in an amount ranging from any of the minimum values described above to any of the maximum values described above, such as 5-120 g ai/ha, 5-90 g ai/ha, 5-75 g ai/ha, 6-114 g ai/ha, 8-108 g ai/ha, 9-113 g ai/ha, 10-80 g ai/ha, 10-55 g ai/ha, 12-112 g ai/ha, 13-85 g ai/ha, 15-106 g ai/ha, 20-100 g ai/ha, 24-105 g ai/ha, or 28-115 g ai/ha.

### Flupyrsulfuron

Compositions and methods of the present disclosure can include flupyrsulfuron or an agriculturally acceptable salt or ester thereof. Flupyrsulfuron-methyl sodium, shown below, is a sulfonylurea that is used, e.g., for post-emergent control of grass and broadleaf weeds in cereals.

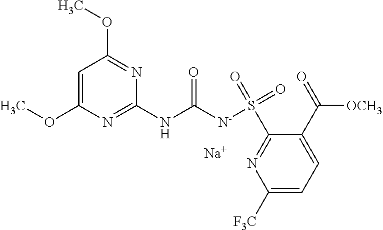 [SMILES: COC(=O)c1ccc(C(F)(F)F)nc1S(=O)(=O)[N-]C(=O)Nc1nc(OC)cc(OC)n1 ; [Na+]]

Flupyrsulfuron, as well as methods of preparing flupyrsulfuron, are known in the art. Its herbicidal activity is described in The Pesticide Manual, Seventeenth Edition, 2016.

Flupyrsulfuron can be applied to vegetation or an area adjacent the vegetation or applied to soil or water to prevent the emergence or growth of vegetation in an amount sufficient to induce a herbicidal effect. In some aspects, flupyrsulfuron thereof is applied to vegetation or an area adjacent the vegetation or applied to soil or water to prevent the emergence or growth of vegetation in an amount of 2.5 g ai/ha or more, such as 2.6 g ai/ha or more, 2.7 g ai/ha or more, 2.8 g ai/ha or more, 2.9 g ai/ha or more, 3.0 g ai/ha or more, 3.2 g ai/ha or more, 3.4 g ai/ha or more, 3.6 g ai/ha or more, 3.8 g ai/ha or more, 4 g ai/ha or more, 4.2 g ai/ha or more, 4.4 g ai/ha or more, 4.6 g ai/ha or more, 4.8 g ai/ha or more, 5 g ai/ha or more, 5.2 g ai/ha or more, 5.4 g ai/ha or more, 5.6 g ai/ha or more, 5.8 g ai/ha or more, 6 g ai/ha or more, 6.2 g ai/ha or more, 6.4 g ai/ha or more, 6.6 g ai/ha or more, 6.8 g ai/ha or more, 7 g ai/ha or more, 7.2 ai/ha or more, 7.4 g ai/ha or more, 7.6 g ai/ha or more, 8 g ai/ha or more, 8.2 g ai/ha or more, 8.4 ai/ha or more, 8.6 g ai/ha or more, 8.8 g ai/ha or more, 9 g ai/ha or more, 9.1 g ai/ha or more, 9.2 g ai/ha or more, 9.3 g ai/ha or more, 9.4 g ai/ha or more, 9.5 g ai/ha or more, 9.6 g ai/ha or more, 9.7 g ai/ha or more, 9.8 g ai/ha or more, or 9.9 g ai/ha or more; in an amount of 10 g ai/ha or less, such as 9.9 g ai/ha or less, 9.8 g ai/ha or less, 9.7 g ai/ha or less, 9.6 g ai/ha or less, 9.5 g ai/ha or less, 9.4 g ai/ha or less, 9.3 g ai/ha or less, 9.2 g ai/ha or less, 9.1 g ai/ha or less, 9 g ai/ha or less, 8.8 g ai/ha or less, 8.6 g ai/ha or less, 8.4 g ai/ha or less, 8.2 g ai/ha or less, 8 g ai/ha or less, 7.8 g ai/ha or less, 7.6 g ai/ha or less, 7.4 g ai/ha or less, 7.2 g ai/ha or less, 7 g ai/ha or less, 6.8 g ai/ha or less, 6.6 g ai/ha or less, 6.4 g ai/ha or less, 6.2 g ai/ha or less, 6 g ai/ha or less, 5.8 g ai/ha or less, 5.6 g ai/ha or less, 5.4 g ai/ha or less, 5.2 g ai/ha or less, 5 g ai/ha or less, 4.8 g ai/ha or less, 4.6 g ai/ha or less, 4.4 g ai/ha or less, 4.2 g ai/ha or less, 4 g ai/ha or less, 3.8 g ai/ha or less, 3.6 g ai/ha or less, 3.4 g ai/ha or less, 3.2 g ai/ha or less, 3 g ai/ha or less, 2.9 g ai/ha or less, 2.8 g ai/ha or less, 2.7 g ai/ha or less, or 2.6 g ai/ha or less; or in an amount ranging from any of the minimum values described above to any of the maximum values described above, such as 2.5-10 g ai/ha, 2.5-9.4 g ai/ha, 2.6-9 g ai/ha, 2.7-10 g ai/ha, 2.8-9.4 g ai/ha, 3-8.8 g ai/ha, 3.2-7.2 g ai/ha, 3.4-8.6 g ai/ha, 3.6-10 g ai/ha, 3.8-9.5 g ai/ha, 4-8.8 g ai/ha, 4.4-7 g ai/ha, 4.6-6.8 g ai/ha, or 5-9.5 g ai/ha.

### Foramsulfuron

Compositions and methods of the present disclosure can include foramsulfuron or an agriculturally acceptable salt or ester thereof. Foramsulfuron, shown below, is a sulfonylurea that provides e.g., post-emergence control of grass and broadleaf weeds in maize. Foramsulfuron, as well as methods of preparing foramsulfuron, are known in the art. Its herbicidal activity is described in The Pesticide Manual, Seventeenth Edition, 2016.

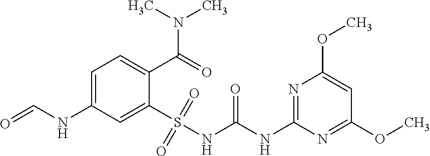 [SMILES: COc1cc(OC)nc(NC(=O)NS(=O)(=O)c2cc(NC=O)ccc2C(=O)N(C)C)n1]

Foramsulfuron can be applied to vegetation or an area adjacent the vegetation or applied to soil or water to prevent the emergence or growth of vegetation in an amount sufficient to induce a herbicidal effect. In some aspects, foramsulfuron is applied to vegetation or an area adjacent the vegetation or applied to soil or water to prevent the emergence or growth of vegetation in an amount of 22.5 g ai/ha or more, such as 23 g ai/ha or more, 24 g ai/ha or more, 25 g ai/ha or more, 26 g ai/ha or more, 28 g ai/ha or more, 30 g ai/ha or more, 32 g ai/ha or more, 34 g ai/ha or more, 35 g ai/ha or more, 36 g ai/ha or more, 38 g ai/ha or more, 40 g ai/ha or more, 42 g ai/ha or more, 44 g ai/ha or more, 46 g ai/ha or more, 48 g ai/ha or more, 50 g ai/ha or more, 51 g ai/ha or more, 52 g ai/ha or more, 53 g ai/ha or more, 54 g ai/ha or more, 55 g ai/ha or more, 56 g ai/ha or more, 57 g ai/ha or more, or 58 g ai/ha or more; in an amount of 59 g ai/ha or less, such as 58 g ai/ha or less, 57 g ai/ha or less, 56 g ai/ha or less, 55 g ai/ha or less, 54 g ai/ha or less, 53 g ai/ha or less, 52 g ai/ha or less, 51 g ai/ha or less, 50 g ai/ha or less, 48 g ai/ha or less, 46 g ai/ha or less, 44 g ai/ha or less, 42 g ai/ha or less, 40 g ai/ha or less, 38 g ai/ha or less, 36 g ai/ha or less, 35 g ai/ha or less 34 g ai/ha or less, 32 g ai/ha or less, 30 g ai/ha or less, 28 g ai/ha or less, 26 g ai/ha or less, 25 g ai/ha or less, 24 g ai/ha or less, or 23 g ai/ha or less; or in an amount ranging from any of the minimum values described above to any of the maximum values described above, such as 22.5-59 g ai/ha, 23-51 g ai/ha, 25-38 g ai/ha, 26-57 g ai/ha, 28-48 g ai/ha, 30-58 g ai/ha, 30-50 g ai/ha, 32-55 g ai/ha, 34-57 g ai/ha, or 35-58 g ai/ha.

### Halosulfuron

Compositions and methods of the present disclosure can include halosulfuron or an agriculturally acceptable salt or ester thereof. Halosulfuron-methyl, shown below, is a sulfonylurea that has demonstrated activity for the control of annual broadleaf weeds and nutsedge species, in maize, sugar cane, rice, sorghum, nuts, and turf. Halosulfuron, as well as methods of preparing halosulfuron, are known in the art. Its herbicidal activity is described in The Pesticide Manual, Seventeenth Edition, 2016.

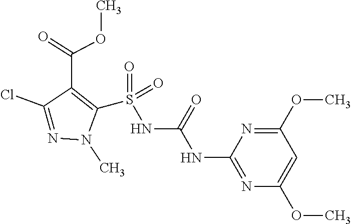 [SMILES: COC(=O)c1c(Cl)nn(C)c1S(=O)(=O)NC(=O)Nc1nc(OC)cc(OC)n1]

Halosulfuron can be applied to vegetation or an area adjacent the vegetation or applied to soil or water to prevent the emergence or growth of vegetation in an amount sufficient to induce a herbicidal effect. In some aspects, halosulfuron is applied to vegetation or an area adjacent the vegetation or applied to soil or water to prevent the emergence or growth of vegetation in an amount of 4.375 g ai/ha or more, such as 4.75 g ai/ha or more, 5 g ai/ha or more, 6 g ai/ha or more, 7 g ai/ha or more, 8 g ai/ha or more, 9 g ai/ha or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 105 g ai/ha or more, 110 g ai/ha or more, 115 g ai/ha or more, 120 g ai/ha or more, 122 g ai/ha or more, 124 g ai/ha or more, 126 g ai/ha or more, 128 g ai/ha or more, 130 g ai/ha or more, 131 g ai/ha or more, 132 g ai/ha or more, 133 g ai/ha or more, 134 g ai/ha or more, 135 g ai/ha or more, 136 g ai/ha or more, 137 g ai/ha or more, 138 g ai/ha or more, or 139 g ai/ha or more; in an amount of 139 g ai/ha or less, such as 138 g ai/ha or less, 137 g ai/ha or less, 136 g ai/ha or less, 135 g ai/ha or less, 134 g ai/ha or less, 133 g ai/ha or less, 132 g ai/ha or less, 131 g ai/ha or less, 130 g ai/ha or less, 129 g ai/ha or less, 128 g ai/ha or less, 126 g ai/ha or less, 124 g ai/ha or less, 122 g ai/ha or less, 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9 g ai/ha or less, 8 g ai/ha or less, 7 g ai/ha or less, 6 g ai/ha or less, 5 g ai/ha or less, 4.75 g ai/ha or less, or 4.5 g ai/ha or less; or in an amount ranging from any of the minimum values described above to any of the maximum values described above, such as 4.375-140 g ai/ha, 4.5-120 g ai/ha, 4.75-100 g ai/ha, 5-95 g ai/ha, 5-131 g ai/ha, 6-80 g ai/ha, 7-130 g ai/ha, 8-122 g ai/ha, 9-75 g ai/ha, 10-139 g ai/ha, 15-90 g ai/ha, 20-124 g ai/ha, 25-110 g ai/ha, or 25-75 g ai/ha.

### Iodosulfuron

Compositions and methods of the present disclosure can include iodosulfuron or an agriculturally acceptable salt or ester thereof. Iodosulfuron-methyl-sodium, shown below, is a sulfonylurea that provides, e.g., post-emergence control of grass and broadleaf weeds in winter, spring and durum wheat, triticale, rye and spring barley. Iodosulfuron, as well as methods of preparing iodosulfuron, are known in the art. Its herbicidal activity is described in The Pesticide Manual, Seventeenth Edition, 2016.

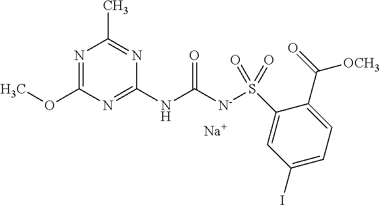 [SMILES: COC(=O)c1ccc(I)cc1S(=O)(=O)[N-]C(=O)Nc1nc(C)nc(OC)n1 ; [Na+]]

Iodosulfuron can be applied to vegetation or an area adjacent the vegetation or applied to soil or water to prevent the emergence or growth of vegetation in an amount sufficient to induce a herbicidal effect. In some aspects, iodosulfuron is applied to vegetation or an area adjacent the vegetation or applied to soil or water to prevent the emergence or growth of vegetation in an amount of 0.1 g ai/ha or more, such as 0.2 g ai/ha or greater, 0.3 g ai/ha or greater, 0.4 g ai/ha or greater, 0.5 g ai/ha or greater, 0.6 g ai/ha or greater, 0.7 g ai/ha or greater, 0.8 g ai/ha or greater, 0.9 g ai/ha or greater, 1 g ai/ha or greater, 1.1 g ai/ha or greater, 1.2 g ai/ha or greater, 1.3 g ai/ha or greater, 1.4 g ai/ha or more, 1.5 g ai/ha or more, 1.6 g ai/ha or more, 1.7 g ai/ha or more, 1.8 g ai/ha or more, 1.9 g ai/ha or more, 2.0 g ai/ha or more, 2.1 g ai/ha or more, 2.2 g ai/ha or more, 2.3 g ai/ha or more, 2.3 g ai/ha or more, 2.4 g ai/ha or more, 2.5 g ai/ha or more, 2.6 g ai/ha or more, 2.7 g ai/ha or more, 2.8 g ai/ha or more, 2.9 g ai/ha or more, 3.0 g ai/ha or more, 3.2 g ai/ha or more, 3.4 g ai/ha or more, 3.6 g ai/ha or more, 3.8 g ai/ha or more, 4 g ai/ha or more, 4.2 g ai/ha or more, 4.4 g ai/ha or more, 4.6 g ai/ha or more, 4.8 g ai/ha or more, 5 g ai/ha or more, 5.2 g ai/ha or more, 5.4 g ai/ha or more, 5.6 g ai/ha or more, 5.8 g ai/ha or more, 6 g ai/ha or more, 6.2 g ai/ha or more, 6.4 g ai/ha or more, 6.6 g ai/ha or more, 6.8 g ai/ha or more, 7 g ai/ha or more, 7.2 ai/ha or more, 7.4 g ai/ha or more, 7.6 g ai/ha or more, 8 g ai/ha or more, 8.2 g ai/ha or more, 8.4 ai/ha or more, 8.6 g ai/ha or more, 8.8 g ai/ha or more, 9 g ai/ha or more, 9.1 g ai/ha or more, 9.2 g ai/ha or more, 9.3 g ai/ha or more, 9.4 g ai/ha or more, 9.5 g ai/ha or more, 9.6 g ai/ha or more, 9.7 g ai/ha or more, 9.8 g ai/ha or more, or 9.9 g ai/ha or more; in an amount of 10 g ai/ha or less, such as 9.9 g ai/ha or less, 9.8 g ai/ha or less, 9.7 g ai/ha or less, 9.6 g ai/ha or less, 9.5 g ai/ha or less, 9.4 g ai/ha or less, 9.3 g ai/ha or less, 9.2 g ai/ha or less, 9.1 g ai/ha or less, 9 g ai/ha or less, 8.8 g ai/ha or less, 8.6 g ai/ha or less, 8.4 g ai/ha or less, 8.2 g ai/ha or less, 8 g ai/ha or less, 7.8 g ai/ha or less, 7.6 g ai/ha or less, 7.4 g ai/ha or less, 7.2 g ai/ha or less, 7 g ai/ha or less, 6.8 g ai/ha or less, 6.6 g ai/ha or less, 6.4 g ai/ha or less, 6.2 g ai/ha or less, 6 g ai/ha or less, 5.8 g ai/ha or less, 5.6 g ai/ha or less, 5.4 g ai/ha or less, 5.2 g ai/ha or less, 5 g ai/ha or less, 4.8 g ai/ha or less, 4.6 g ai/ha or less, 4.4 g ai/ha or less, 4.2 g ai/ha or less, 4 g ai/ha or less, 3.8 g ai/ha or less, 3.6 g ai/ha or less, 3.4 g ai/ha or less, 3.2 g ai/ha or less, 3 g ai/ha or less, 2.9 g ai/ha or less, 2.8 g ai/ha or less, 2.7 g ai/ha or less, 2.6 g ai/ha or less, 2.5 g ai/ha or less, 2.4 g ai/ha or less, 2.3 g ai/ha or less, 2.2 g ai/ha or less, 2.1 g ai/ha or less, 2.0 g ai/ha or less, 1.9 g ai/ha or less, 1.8 g ai/ha or less, 1.7 g ai/ha or less, 1.6 g ai/ha or less, 1.5 g ai/ha or less, 1.4 g ai/ha or less, 1.3 g ai/ha or less, 1.2 g ai/ha or less, 1.1 g ai/ha or less, 1 g ai/ha or less, 0.9 g ai/ha or less, 0.8 g ai/ha or less, 0.7 g ai/ha or less, 0.6 g ai/ha or less, 0.5 g ai/ha or less, 0.4 g ai/ha or less, 0.3 g ai/ha or less, or 0.2 g ai/ha or less; or in an amount ranging from any of the minimum values described above to any of the maximum values described above, such as 0.1-10 g ai/ha, 0.4-8.8 g ai/ha, 1.1-6.2 g ai/ha, 1.7-7.5 g ai/ha, 1.8-9.2 g ai/ha, 1.9-9 g ai/ha, 2-6.8 g ai/ha, 2.2-9.5 g ai/ha, 2.5-10 g ai/ha, 2.8-7.6 g ai/ha, 3-9.4 g ai/ha, 3.2-7.2 g ai/ha, 3.4-10 g ai/ha, or 3.6-8 g ai/ha.

### Mesosulfuron

Compositions and methods of the present disclosure can include mesosulfuron or an agriculturally acceptable salt or ester thereof. Mesosulfuron-methyl, shown below, is a sulfonylurea that provides, e.g., early- to mid-post-emergence control of grass and some broadleaf weeds in winter, spring and durum wheat, triticale and rye. Mesosulfuron, as well as methods of preparing mesosulfuron, are known in the art. Its herbicidal activity is described in The Pesticide Manual, Seventeenth Edition, 2016.

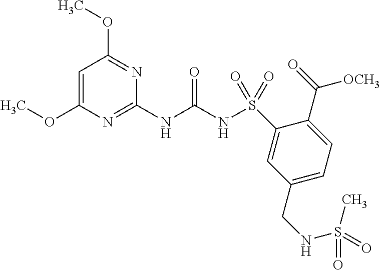 [SMILES: COC(=O)c1ccc(CNS(C)(=O)=O)cc1S(=O)(=O)NC(=O)Nc1nc(OC)cc(OC)n1]

Mesosulfuron can be applied to vegetation or an area adjacent the vegetation or applied to soil or water to prevent the emergence or growth of vegetation in an amount sufficient to induce a herbicidal effect. In some aspects, mesosulfuron thereof is applied to vegetation or an area adjacent the vegetation or applied to soil or water to prevent the emergence or growth of vegetation in an amount of 1.5 g ai/ha or more, such as 1.6 g ai/ha or more, 1.7 g ai/ha or more, 1.8 g ai/ha or more, 1.9 g ai/ha or more, 2 g ai/ha or more, 2.1 g ai/ha or more, 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.25 g ai/ha or more, 4.5 g ai/ha or more, 4.75 g ai/ha or more, 5 g ai/ha or more, 6 g ai/ha or more, 7 g ai/ha or more, 8 g ai/ha or more, 9 g ai/ha or more, 10 g ai/ha or more, 12 g ai/ha or more, 14 g ai/ha or more, 16 g ai/ha or more, 18 g ai/ha or more, 20 g ai/ha or more, 22 g ai/ha or more, 24 g ai/ha or more, 26 g ai/ha or more, 28 g ai/ha or more, 30 g ai/ha or more, 31 g ai/ha or more, 32 g ai/ha or more, 33 g ai/ha or more, 34 g ai/ha or more, 35 g ai/ha or more, 36 g ai/ha or more, 37 g ai/ha or more, 38 g ai/ha or more, 39 g ai/ha or more, 40 g ai/ha or more, 41 g ai/ha or more, 42 g ai/ha or more, 43 g ai/ha or more, or 44 g ai/ha or more; in an amount of 45 g ai/ha or less, such as 44 g ai/ha or less, 43 g ai/ha or less, 42 g ai/ha or less, 41 g ai/ha or less, 40 g ai/ha or less, 39 g ai/ha or less, 38 g ai/ha or less, 37 g ai/ha or less, 36 g ai/ha or less, 35 g ai/ha or less, 34 g ai/ha or less, 33 g ai/ha or less, 32 g ai/ha or less, 31 g ai/ha or less, 30 g ai/ha or less, 28 g ai/ha or less, 26 g ai/ha or less, 24 g ai/ha or less, 22 g ai/ha or less, 20 g ai/ha or less, 18 g ai/ha or less, 16 g ai/ha or less, 14 g ai/ha or less, 12 g ai/ha or less, 10 g ai/ha or less, 9 g ai/ha or less, 8 g ai/ha or less, 7 g ai/ha or less, 6 g ai/ha or less, 5 g ai/ha or less, 4.75 g ai/ha or less, 4.5 g ai/ha or less, 4.25 g ai/ha or less, 4 g ai/ha or less, 3.75 g ai/ha or less, 3.5 g ai/ha or less, 3.25 g ai/ha or less, 3 g ai/ha or less, 2.75 g ai/ha or less, 2.5 g ai/ha or less, 2.25 g ai/ha or less, 2.1 g ai/ha or less, 2 g ai/ha or less, 1.9 g ai/ha or less, 1.8 g ai/ha or less, 1.7 g ai/ha or less, 1.6 g ai/ha or less; or in an amount ranging from any of the minimum values described above to any of the maximum values described above, such as 1.5-45 g ai/ha, 1.6-42 g ai/ha, 1.5-40 g ai/ha, 1.7-43 g ai/ha, 1.8-35 g ai/ha, 2-37 g ai/ha, 2.25-41 g ai/ha, 2.5-32 g ai/ha, 2.75-45 g ai/ha, 3-40 g ai/ha, 3-33 g ai/ha, 3-24 g ai/ha, 3.5-45 g ai/ha, or 4-39 g ai/ha.

### Metsulfuron

Compositions and methods of the present disclosure can include metsulfuron or an agriculturally acceptable salt or ester thereof. Metsulfuron-methyl, shown below, is a sulfonylurea that controls, e.g., grass and broadleaf weeds in wheat, barley, rice, oats and triticale. Metsulfuron, as well as methods of preparing metsulfuron, are known in the art. Its herbicidal activity is described in The Pesticide Manual, Seventeenth Edition, 2016.

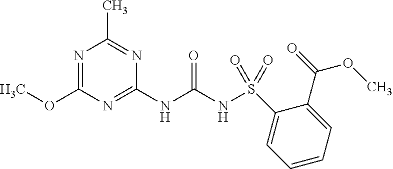

Metsulfuron can be applied to vegetation or an area adjacent the vegetation or applied to soil or water to prevent the emergence or growth of vegetation in an amount sufficient to induce a herbicidal effect. In some aspects, metsulfuron thereof is applied to vegetation or an area adjacent the vegetation or applied to soil or water to prevent the emergence or growth of vegetation in an amount of 1 g ai/ha or more, such as 1.1 g ai/ha or more, 1.2 g ai/ha or more, 1.25 g ai/ha or more, 1.3 g ai/ha or more, 1.4 g ai/ha or more, 1.5 g ai/ha or more, 1.6 g ai/ha or more, 1.7 g ai/ha or more, 1.8 g ai/ha or more, 1.9 g ai/ha or more, 2 g ai/ha or more, 2.1 g ai/ha or more, 2.2 g ai/ha or more, 2.3 g ai/ha or more, 2.3 g ai/ha or more, 2.4 g ai/ha or more, 2.5 g ai/ha or more, 2.6 g ai/ha or more, 2.7 g ai/ha or more, 2.8 g ai/ha or more, 2.9 g ai/ha or more, 3 g ai/ha or more, 3.2 g ai/ha or more, 3.4 g ai/ha or more, 3.6 g ai/ha or more, 3.8 g ai/ha or more, 4 g ai/ha or more, 4.2 g ai/ha or more, 4.4 g ai/ha or more, 4.6 g ai/ha or more, 4.8 g ai/ha or more, 5 g ai/ha or more, 5.2 g ai/ha or more, 5.4 g ai/ha or more, 5.6 g ai/ha or more, 5.8 g ai/ha or more, 6 g ai/ha or more, 6.2 g ai/ha or more, 6.4 g ai/ha or more, 6.6 g ai/ha or more, 6.8 g ai/ha or more, 7 g ai/ha or more, 7.2 ai/ha or more, 7.4 g ai/ha or more, 7.6 g ai/ha or more, 7.8 g ai/ha or more, 8 g ai/ha or more, 8.2 g ai/ha or more, 8.4 ai/ha or more, 8.6 g ai/ha or more, 8.8 g ai/ha or more, 9 g ai/ha or more, 9.2 g ai/ha or more, 9.4 g ai/ha or more, 9.6 g ai/ha or more, 9.8 g ai/ha or more, 10 g ai/ha or more, 10.2 g ai/ha or more, 10.4 g ai/ha or more, 10.6 g ai/ha or more, 10.8 g ai/ha or more, 11 g ai/ha or more, 11.1 g ai/ha or more, 11.2 g ai/ha or more, 11.3 g ai/ha or more, 11.4 g ai/ha or more, 11.5 g ai/ha or more, 11.6 g ai/ha or more, 11.7 g ai/ha or more, 11.8 g ai/ha or more, or 11.9 g ai/ha or more; in an amount of 12 g ai/ha or less, such as 11.9 g ai/ha or less, 11.8 g ai/ha or less, 11.7 g ai/ha or less, 11.6 g ai/ha or less, 11.5 g ai/ha or less, 11.4 g ai/ha or less, 11.3 g ai/ha or less, 11.2 g ai/ha or less, 11.1 g ai/ha or less, 11 g ai/ha or less, 10.8 g ai/ha or less, 10.6 g ai/ha or less, 10.4 g ai/ha or less, 10.2 g ai/ha or less, 10 g ai/ha or less, 9.8 g ai/ha or less, 9.6 g ai/ha or less, 9.4 g ai/ha or less, 9.2 g ai/ha or less, 9 g ai/ha or less, 8.8 g ai/ha or less, 8.6 g ai/ha or less, 8.4 g ai/ha or less, 8.2 g ai/ha or less, 8 g ai/ha or less, 7.8 g ai/ha or less, 7.6 g ai/ha or less, 7.4 g ai/ha or less, 7.2 g ai/ha or less, 7 g ai/ha or less, 6.8 g ai/ha or less, 6.6 g ai/ha or less, 6.4 g ai/ha or less, 6.2 g ai/ha or less, 6 g ai/ha or less, 5.8 g ai/ha or less, 5.6 g ai/ha or less, 5.4 g ai/ha or less, 5.2 g ai/ha or less, 5 g ai/ha or less, 4.8 g ai/ha or less, 4.6 g ai/ha or less, 4.4 g ai/ha or less, 4.2 g ai/ha or less, 4 g ai/ha or less, 3.8 g ai/ha or less, 3.6 g ai/ha or less, 3.4 g ai/ha or less, 3.2 g ai/ha or less, 3 g ai/ha or less, 2.9 g ai/ha or less, 2.8 g ai/ha or less, 2.7 g ai/ha or less, 2.6 g ai/ha or less, 2.5 g ai/ha or less, 2.4 g ai/ha or less, 2.3 g ai/ha or less, 2.2 g ai/ha or less, 2.1 g ai/ha or less, 2 g ai/ha or less, 1.9 g ai/ha or less, 1.8 g ai/ha, 1.7 g ai/ha or less, 1.6 g ai/ha or less, 1.5 g ai/ha or less, 1.4 g ai/ha or less, 1.3 g ai/ha or less, 1.25 g ai/ha or less, 1.2 g ai/ha or less, or 1.1 g ai/ha or less; or in an amount ranging from any of the minimum values described above to any of the maximum values described above, such as 1-12 g ai/ha, 1-10 g ai/ha, 1.1-9.4 g ai/ha, 1.25-8.2 g ai/ha, 1.25-11 g ai/ha, 1.3-7 g ai/ha, 1.4-5.8 g ai/ha, 1.5-12 g ai/ha, 1.5-10.4 g ai/ha, 1.5-8.8 g ai/ha, 2-11.7 g ai/ha, 2.2-6.4 g ai/ha, 2.5-7.6 g ai/ha, 2.8-12 g ai/ha, or 3-8 g ai/ha.

### Nicosulfuron

Compositions and methods of the present disclosure can include nicosulfuron or an agriculturally acceptable salt or ester thereof. Nicosulfuron, shown below, is a sulfonylurea that provides e.g., selective post-emergence control in maize of annual grass weeds, including Setaria, Echinochloa, Digitaria, Panicum, Lolium and Avena spp., broadleaf weeds, including Amaranthus spp. and Cruciferae, and perennials such as Sorghum halepense and Agropyron repens. Nicosulfuron, as well as methods of preparing nicosulfuron, are known in the art. Its herbicidal activity is described in The Pesticide Manual, Seventeenth Edition, 2016.

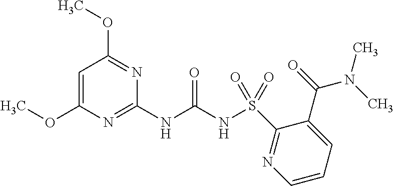

Nicosulfuron can be applied to vegetation or an area adjacent the vegetation or applied to soil or water to prevent the emergence or growth of vegetation in an amount sufficient to induce a herbicidal effect. In some aspects, nicosulfuron is applied to vegetation or an area adjacent the vegetation or applied to soil or water to prevent the emergence or growth of vegetation in an amount of 8.75 g ai/ha or more, such as 9 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 17 g ai/ha or more, 18 g ai/ha or more, 19 g ai/ha or more, 20 g ai/ha or more, 22 g ai/ha or more, 24 g ai/ha or more, 26 g ai/ha or more, 28 g ai/ha or more, 30 g ai/ha or more, 32 g ai/ha or more, 34 g ai/ha or more, 36 g ai/ha or more, 38 g ai/ha or more, 40 g ai/ha or more, 42 g ai/ha or more, 44 g ai/ha or more, 46 g ai/ha or more, 48 g ai/ha or more, 50 g ai/ha or more, 52 g ai/ha or more, 54 g ai/ha or more, 56 g ai/ha or more, 58 g ai/ha or more, 60 g ai/ha or more, 62 g ai/ha or more, 64 g ai/ha or more, 66 g ai/ha or more, 68 g ai/ha or more, or 69 g ai/ha or more; in an amount of 70 g ai/ha or less, such as 69 g ai/ha or less, 68 g ai/ha or less, 66 g ai/ha or less, 64 g ai/ha or less, 62 g ai/ha or less, 60 g ai/ha or less, 58 g ai/ha or less, 56 g ai/ha or less, 54 g ai/ha or less, 52 g ai/ha or less, 50 g ai/ha or less, 48 g ai/ha or less, 46 g ai/ha or less, 44 g ai/ha or less, 42 g ai/ha or less, 40 g ai/ha or less, 38 g ai/ha or less, 36 g ai/ha or less, 34 g ai/ha or less, 32 g ai/ha or less, 30 g ai/ha or less, 28 g ai/ha or less, 26 g ai/ha or less, 24 g ai/ha or less, 22 g ai/ha or less, 20 g ai/ha or less, 19 g ai/ha or less, 18 g ai/ha or less, 17 g ai/ha or less, 16 g ai/ha or less, 15 g ai/ha or less, 14 g ai/ha or less, 13 g ai/ha or less, 12 g ai/ha or less, 11 g ai/ha or less, 10 g ai/ha or less, or 9 g ai/ha or less; or in an amount ranging from any of the minimum values described above to any of the maximum values described above, such as 8.75-70 g ai/ha, 9-68 g ai/ha, 10-50 g ai/ha, 12-66 g ai/ha, 14-44 g ai/ha, 15-58 g ai/ha, 18-65 g ai/ha, 20-45 g ai/ha, 22-60 g ai/ha, 26-56 g ai/ha, or 30-70 g ai/ha.

### Orthosulfamuron

Compositions and methods of the present disclosure can include orthosulfamuron or an agriculturally acceptable salt or ester thereof. Orthosulfamuron, shown below, is a sulfonylurea that provides e.g., early post-emergence control of annual and perennial broadleaf weeds and sedges in rice, cereals, pastures and sugar cane. Orthosulfamuron, as well as methods

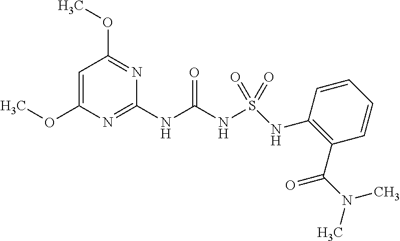

of preparing orthosulfamuron, are known in the art. Its herbicidal activity is described, for example, in The Pesticide Manual, Seventeenth Edition, 2016.

Orthosulfamuron can be applied to vegetation or an area adjacent the vegetation or applied to soil or water to prevent the emergence or growth of vegetation in an amount sufficient to induce a herbicidal effect. In some aspects, orthosulfamuron is applied to vegetation or an area adjacent the vegetation or applied to soil or water to prevent the emergence or growth of vegetation in an amount of 7.5 g ai/ha or more, such as 8 g ai/ha or more, 9 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 17 g ai/ha or more, 18 g ai/ha or more, 19 g ai/ha or more, 20 g ai/ha or more, 22 g ai/ha or more, 24 g ai/ha or more, 26 g ai/ha or more, 28 g ai/ha or more, 30 g ai/ha or more, 32 g ai/ha or more, 34 g ai/ha or more, 36 g ai/ha or more, 38 g ai/ha or more, 40 g ai/ha or more, 42 g ai/ha or more, 44 g ai/ha or more, 46 g ai/ha or more, 48 g ai/ha or more, 50 g ai/ha or more, 52 g ai/ha or more, 54 g ai/ha or more, 56 g ai/ha or more, 58 g ai/ha or more, 60 g ai/ha or more, 62 g ai/ha or more, 64 g ai/ha or more, 66 g ai/ha or more, 68 g ai/ha or more, 70 g ai/ha or more, 71 g ai/ha or more, 72 g ai/ha or more, 73 g ai/ha or more, or 74 g ai/ha or more; in an amount of 75 g ai/ha or less, such as 74 g ai/ha or less, 72 g ai/ha or less, 70 g ai/ha or less, 68 g ai/ha or less, 66 g ai/ha or less, 64 g ai/ha or less, 62 g ai/ha or less, 60 g ai/ha or less, 58 g ai/ha or less, 56 g ai/ha or less, 54 g ai/ha or less, 52 g ai/ha or less, 50 g ai/ha or less, 48 g ai/ha or less, 46 g ai/ha or less, 44 g ai/ha or less, 42 g ai/ha or less, 40 g ai/ha or less, 38 g ai/ha or less, 36 g ai/ha or less, 34 g ai/ha or less, 32 g ai/ha or less, 30 g ai/ha or less, 28 g ai/ha or less, 26 g ai/ha or less, 24 g ai/ha or less, 22 g ai/ha or less, 20 g ai/ha or less, 19 g ai/ha or less, 18 g ai/ha or less, 17 g ai/ha or less, 16 g ai/ha or less, 15 g ai/ha or less, 14 g ai/ha or less, 13 g ai/ha or less, 12 g ai/ha or less, 11 g ai/ha or less, 10 g ai/ha or less, 9 g ai/ha or less, or 8 g ai/ha or less; or in an amount ranging from any of the minimum values described above to any of the maximum values described above, such as 7.5-75 g ai/ha, 7.5-60 g ai/ha, 8-58 g ai/ha, 9-42 g ai/ha, 10-70 g ai/ha, 10-56 g ai/ha, 11-40 g ai/ha, 13-64 g ai/ha, 15-75 g ai/ha, 17-46 g ai/ha, 20-65 g ai/ha, 20-52 g ai/ha, 22-48 g ai/ha, or 24-74 g ai/ha.

### Rimsulfuron

Compositions and methods of the present disclosure can include rimsulfuron or an agriculturally acceptable salt or ester thereof. Rimsulfuron, shown below, is a sulfonylurea that provides post-emergence control of most annual and perennial grass weeds and several broad-leaved weeds in maize, tomatoes and potatoes, at 15 g/ha. Rimsulfuron, as well as methods of preparing rimsulfuron, are known in the art. Its herbicidal activity is described in The Pesticide Manual, Seventeenth Edition, 2016.

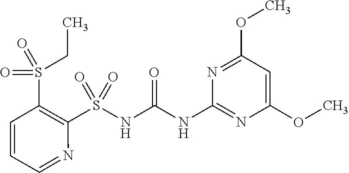 [SMILES: CCS(=O)(=O)c1cccnc1S(=O)(=O)NC(=O)Nc1nc(OC)cc(OC)n1]

Rimsulfuron can be applied to vegetation or an area adjacent the vegetation or applied to soil or water to prevent the emergence or growth of vegetation in an amount sufficient to induce a herbicidal effect. In some aspects, rimsulfuron is applied to vegetation or an area adjacent the vegetation or applied to soil or water to prevent the emergence or growth of vegetation in an amount of 5 g ai/ha or more, such as 5.5 g ai/ha or more, 6 g ai/ha or more, 7 g ai/ha or more, 7.5 g ai/ha or more, 8 g ai/ha or more, 9 g ai/ha or more, 10 g ai/ha or more, 12 g ai/ha or more, 14 g ai/ha or more, 16 g ai/ha or more, 18 g ai/ha or more, 20 g ai/ha or more, 22 g ai/ha or more, 24 g ai/ha or more, 26 g ai/ha or more, 27 g ai/ha or more, 28 g ai/ha or more, 30 g ai/ha or more, 32 g ai/ha or more, 34 g ai/ha or more, 36 g ai/ha or more, 38 g ai/ha or more, 40 g ai/ha or more, 42 g ai/ha or more, 44 g ai/ha or more, 46 g ai/ha or more, 48 g ai/ha or more, 50 g ai/ha or more, 52 g ai/ha or more, 54 g ai/ha or more, 56 g ai/ha or more, 58 g ai/ha or more, 60 g ai/ha or more, 62 g ai/ha or more, 64 g ai/ha or more, 66 g ai/ha or more, 68 g ai/ha or more, 69 g ai/ha or more, 70 g ai/ha or more, 71 g ai/ha or more, 72 g ai/ha or more, 73 g ai/ha or more, or 74 g ai/ha or more; in an amount of 75 g ai/ha or less, such as 74 g ai/ha or less, 73 g ai/ha or less, 72 g ai/ha or less, 71 g ai/ha or less, 70 g ai/ha or less, 69 g ai/ha or less, 68 g ai/ha or less, 66 g ai/ha or less, 64 g ai/ha or less, 62 g ai/ha or less, 60 g ai/ha or less, 58 g ai/ha or less, 56 g ai/ha or less, 54 g ai/ha or less, 52 g ai/ha or less, 50 g ai/ha or less, 48 g ai/ha or less, 46 g ai/ha or less, 44 g ai/ha or less, 42 g ai/ha or less, 40 g ai/ha or less, 38 g ai/ha or less, 36 g ai/ha or less, 34 g ai/ha or less, 32 g ai/ha or less, 30 g ai/ha or less, 28 g ai/ha or less, 27 g ai/ha or less, 26 g ai/ha or less, 24 g ai/ha or less, 22 g ai/ha or less, 20 g ai/ha or less, 18 g ai/ha or less, 16 g ai/ha or less, 14 g ai/ha or less, 12 g ai/ha or less, 10 g ai/ha or less, 9 g ai/ha or less, 8 g ai/ha or less, 7.5 g ai/ha or less, 7 g ai/ha or less, 6 g ai/ha or less, 5.5 g ai/ha or less, or 5 g ai/ha or less; or in an amount ranging from any of the minimum values described above to any of the maximum values described above, such as 5-75 g ai/ha, 7.5-70 g ai/ha, 9-64 g ai/ha, 18-69 g ai/ha, 27-36 g ai/ha, 5.5-66 g ai/ha, 14-18 g ai/ha, 27-69 g ai/ha, 7-38 g ai/ha, 12-52 g ai/ha, 9-44 g ai/ha, 10-38 g ai/ha, 16-75 g ai/ha, or 20-48 g ai/ha.

### Sulfosulfuron

Compositions and methods of the present disclosure can include sulfosulfuron or an agriculturally acceptable salt or ester thereof. Sulfosulfuron, shown below, is a sulfonylurea that provides e.g., control of annual broadleaf weeds and grass weeds in cereals. Sulfosulfuron, as well as methods of preparing sulfosulfuron, are known in the art. Its herbicidal activity is described in The Pesticide Manual, Seventeenth Edition, 2016.

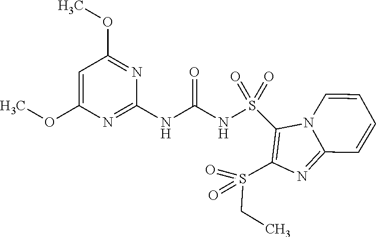 [SMILES: CCS(=O)(=O)c1nc2ccccn2c1S(=O)(=O)NC(=O)Nc1nc(OC)cc(OC)n1]

Sulfosulfuron can be applied to vegetation or an area adjacent the vegetation or applied to soil or water to prevent the emergence or growth of vegetation in an amount sufficient to induce a herbicidal effect. In some aspects, sulfosulfuron is applied to vegetation or an area adjacent the vegetation or applied to soil or water to prevent the emergence or growth of vegetation in an amount of 4.375 g ai/ha or more, such as 4.75 g ai/ha or more, 5 g ai/ha or more, 6 g ai/ha or more, 7 g ai/ha or more, 8 g ai/ha or more, 9 g ai/ha or more, 10 g ai/ha or more, 12 g ai/ha or more, 14 g ai/ha or more, 16 g ai/ha or more, 18 g ai/ha or more, 20 g ai/ha or more, 22 g ai/ha or more, 24 g ai/ha or more, 26 g ai/ha or more, 28 g ai/ha or more, 30 g ai/ha or more, 32 g ai/ha or more, 34 g ai/ha or more, 36 g ai/ha or more, 38 g ai/ha or more, 40 g ai/ha or more, 42 g ai/ha or more, 44 g ai/ha or more, 46 g ai/ha or more, 48 g ai/ha or more, 50 g ai/ha or more, 52 g ai/ha or more, 54 g ai/ha or more, 56 g ai/ha or more, 58 g ai/ha or more, 60 g ai/ha or more, 62 g ai/ha or more, 64 g ai/ha or more, 66 g ai/ha or more, 68 g ai/ha or more, 70 g ai/ha or more, 71 g ai/ha or more, 72 g ai/ha or more, 73 g ai/ha or more, or 74 g ai/ha or more; in an amount of 75 g ai/ha or less, such as 74 g ai/ha or less, 73 g ai/ha or less, 72 g ai/ha or less, 71 g ai/ha or less, 70 g ai/ha or less, 68 g ai/ha or less, 66 g ai/ha or less, 64 g ai/ha or less, 62 g ai/ha or less, 60 g ai/ha or less, 58 g ai/ha or less, 56 g ai/ha or less, 54 g ai/ha or less, 52 g ai/ha or less, 50 g ai/ha or less, 48 g ai/ha or less, 46 g ai/ha or less, 44 g ai/ha or less, 42 g ai/ha or less, 40 g ai/ha or less, 38 g ai/ha or less, 36 g ai/ha or less, 34 g ai/ha or less, 32 g ai/ha or less, 30 g ai/ha or less, 28 g ai/ha or less, 26 g ai/ha or less, 24 g ai/ha or less, 22 g ai/ha or less, 20 g ai/ha or less, 18 g ai/ha or less, 16 g ai/ha or less, 14 g ai/ha or less, 12 g ai/ha or less, 10 g ai/ha or less, 9 g ai/ha or less, 8 g ai/ha or less, 7 g ai/ha or less, 6 g ai/ha or less, 5 g ai/ha or less, or 4.75 g ai/ha or less, or 4.5 g ai/ha or less; or in an amount ranging from any of the minimum values described above to any of the maximum values described above such as 4.375-75 g ai/ha, 4.5-70 g ai/ha, 4.75-64 g ai/ha, 5-75 g ai/ha, 5-73 g ai/ha, 5-66 g ai/ha, 5-40 g ai/ha, 6-75 g ai/ha, 7-68 g ai/ha, 7-52 g ai/ha, 9-44 g ai/ha, 10-50 g ai/ha, 16-75 g ai/ha, or 20-60 g ai/ha.

### Thifensulfuron

Compositions and methods of the present disclosure can include thifensulfuron or an agriculturally acceptable salt or ester thereof. Thifensulfuron-methyl, shown below, is a sulfonylurea that provides e.g., control of annual weeds in cereals, maize and pasture. Thifensulfuron, as well as methods of preparing thifensulfuron, are known in the art. Its herbicidal activity is described in The Pesticide Manual, Seventeenth Edition, 2016.

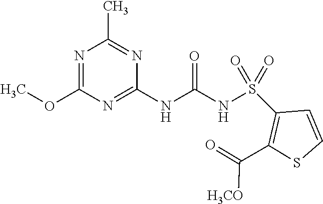 [SMILES: COC(=O)c1sccc1S(=O)(=O)NC(=O)Nc1nc(C)nc(OC)n1]

Thifensulfuron can be applied to vegetation or an area adjacent the vegetation or applied to soil or water to prevent the emergence or growth of vegetation in an amount sufficient to induce a herbicidal effect. In some aspects, thifensulfuron is applied to vegetation or an area adjacent the vegetation or applied to soil or water to prevent the emergence or growth of vegetation in an amount of 4.4 g ai/ha or more, such as 4.5 g ai/ha or more, 4.6 g ai/ha or more, 4.7 g ai/ha or more, 4.8 g ai/ha or more, 4.9 g ai/ha or more, 5 g ai/ha or more, 5.2 g ai/ha or more, 5.4 g ai/ha or more, 5.6 g ai/ha or more, 5.8 g ai/ha or more, 6 g ai/ha or more, 6.2 g ai/ha or more, 6.4 g ai/ha or more, 6.6 g ai/ha or more, 6.8 g ai/ha or more, 7 g ai/ha or more, 7.2 g ai/ha or more, 7.4 g ai/ha or more, 7.6 g ai/ha or more, 7.8 g ai/ha or more, 8 g ai/ha or more, 8.2 g ai/ha or more, 8.4 g ai/ha or more, 8.6 g ai/ha or more, 8.8 g ai/ha or more, 9 g ai/ha or more, 9.2 g ai/ha or more, 9.4 g ai/ha or more, 9.6 g ai/ha or more, 9.8 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 16.2 g ai/ha or more, 16.4 g ai/ha or more, 16.6 g ai/ha or more, 16.8 g ai/ha or more, 17 g ai/ha or more, 17.1 g ai/ha or more, 17.2 g ai/ha or more, 17.3 g ai/ha or more, or 17.4 g ai/ha or more; in an amount of 17.5 g ai/ha or less, such as 17.4 g ai/ha or less, 17.3 g ai/ha or less, 17.2 g ai/ha or less, 17.1 g ai/ha or less, 17 g ai/ha or less, 16.8 g ai/ha or less, 16.6 g ai/ha or less, 16.4 g ai/ha or less, 16.2 g ai/ha or less, 16 g ai/ha or less, 15 g ai/ha or less, 14 g ai/ha or less, 13 g ai/ha or less, 12 g ai/ha or less, 11 g ai/ha or less, 10 g ai/ha or less, 9.8 g ai/ha or less, 9.6 g ai/ha or less, 9.4 g ai/ha or less, 9.2 g ai/ha or less, 9 g ai/ha or less, 8.8 g ai/ha or less, 8.6 g ai/ha or less, 8.4 g ai/ha or less, 8.2 g ai/ha or less, 8 g ai/ha or less, 7.8 g ai/ha or less, 7.6 g ai/ha or less, 7.4 g ai/ha or less, 7.2 g ai/ha or less, 7 g ai/ha or less, 6.8 g ai/ha or less, 6.6 g ai/ha or less, 6.4 g ai/ha or less, 6.2 g ai/ha or less, 6 g ai/ha or less, 5.8 g ai/ha or less, 5.6 g ai/ha or less, 5.4 g ai/ha or less, 5.2 g ai/ha or less, 5 g ai/ha or less, 4.9 g ai/ha or less, 4.8 g ai/ha or less, 4.7 g ai/ha or less, 4.6 g ai/ha or less, 4.5 g ai/ha or less, or 4.4 g ai/ha or less; or in an amount ranging from any of the minimum values described above to any of the maximum values described above, such as 4.4-17.5 g ai/ha, 4.5-16 g ai/ha, 4.8-11 g ai/ha, 5-17.3 g ai/ha, 5-16.2 g ai/ha, 5.4-13 g ai/ha, 5.8-10 g ai/ha, 6-12 g ai/ha, 6-16 g ai/ha, 7.4-16.4 g ai/ha, 7.8-15 g ai/ha, 8-15 g ai/ha, or 8-17.1 g ai/ha.

### Tribenuron

Compositions and methods of the present disclosure can include tribenuron or an agriculturally acceptable salt or ester thereof. Tribenuron-methyl, shown below, is a sulfonylurea that provides e.g., post-emergence control of broadleaf weeds in cereal crops, including wheat, barley, oats, rye and triticale. Tribenuron, as well as methods of preparing tribenuron, are known in the art. Its herbicidal activity is described in The Pesticide Manual,

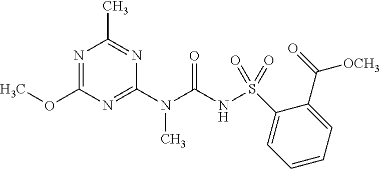 [SMILES: COC(=O)c1ccccc1S(=O)(=O)NC(=O)N(C)c1nc(C)nc(OC)n1]

### Seventeenth Edition, 2016.

Tribenuron can be applied to vegetation or an area adjacent the vegetation or applied to soil or water to prevent the emergence or growth of vegetation in an amount sufficient to induce a herbicidal effect. In some aspects, tribenuron is applied to vegetation or an area adjacent the vegetation or applied to soil or water to prevent the emergence or growth of vegetation in an amount of 3.75 g ai/ha or more, such as 4 g ai/ha or more, 4.25 g ai/ha or more, 4.5 g ai/ha or more, 4.75 g ai/ha or more, 5 g ai/ha or more, 5.25 g ai/ha or more, 5.5 g ai/ha or more, 5.75 g ai/ha or more, 6 g ai/ha or more, 7 g ai/ha or more, 7.5 g ai/ha or more, 8 g ai/ha or more, 9 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 17 g ai/ha or more, 18 g ai/ha or more, 19 g ai/ha or more, 20 g ai/ha or more, 21 g ai/ha or more, 22 g ai/ha or more, 23 g ai/ha or more, 24 g ai/ha or more, 25 g ai/ha or more, 25.5 g ai/ha or more, 26 g ai/ha or more, 26.5 g ai/ha or more, 27 g ai/ha or more, 27.5 g ai/ha or more, 28 g ai/ha or more, 28.5 g ai/ha or more, 29 g ai/ha or more, 29.25 g ai/ha or more, 29.5 g ai/ha or more, or 29.75 g ai/ha or more; in an amount of 30 g ai/ha or less, such as 29.75 g ai/ha or less, 29.5 g ai/ha or less, 29.25 g ai/ha or less, 29 g ai/ha or less, 28.5 g ai/ha or less, 28 g ai/ha or less, 27.5 g ai/ha or less, 27 g ai/ha or less, 26.5 g ai/ha or less, 26 g ai/ha or less, 25 g ai/ha or less, 24 g ai/ha or less, 23 g ai/ha or less, 22 g ai/ha or less, 21 g ai/ha or less, 20 g ai/ha or less, 19 g ai/ha or less, 18 g ai/ha or less, 17 g ai/ha or less, 16 g ai/ha or less, 15 g ai/ha or less, 14 g ai/ha or less, 13 g ai/ha or less, 12 g ai/ha or less, 11 g ai/ha or less, 10 g ai/ha or less, 9 g ai/ha or less, 8 g ai/ha or less, 7.5 g ai/ha or less, 7 g ai/ha or less, 6 g ai/ha or less, 5.75 g ai/ha or less, 5.5 g ai/ha or less, 5.25 g ai/ha or less, 5 g ai/ha or less, 4.75 g ai/ha or less, 4.5 g ai/ha or less, 4.25 g ai/ha or less, or 4 g ai/ha or less; or in an amount ranging from any of the minimum values described above to any of the maximum values described above such as 3.75-30 g ai/ha, 4-27.5 g ai/ha, 4.25-23 g ai/ha, 4.5-17 g ai/ha, 5-25 g ai/ha, 6-29.5 g ai/ha, 7-28 g ai/ha, 7.5-23 g ai/ha, 7.5-20 g ai/ha, 9-30 g ai/ha, 10-19 g ai/ha, or 10-27.5 g ai/ha.

### Synthetic Auxin Herbicides

In addition to the compounds according to Formula I, the compositions can include a synthetic auxin herbicide. Synthetic auxin herbicides mimic natural plant hormones and can inhibit cell division and growth. Synthetic auxin herbicides include phenoxy herbicides, benzoic acid herbicides, aryl picolinate herbicides, compounds according to Formula Is, quinoline carboxylic acid herbicides, pyrimidine carboxylic acid herbicides, and benzothiazole herbicides, as well as agriculturally acceptable salts and esters thereof.

In some aspects, the composition can include a synthetic auxin herbicide selected from the group consisting of 2,4-D; 2,4-DB; 2,3,6-TBA, aminocyclopyrachlor, aminopyralid, benazolin-ethyl, chloramben, clomeprop, clopyralid, dichlorprop, dichlorprop-P, dicamba, florpyrauxifen (such as florpyrauxifen-benzyl), fluchloraminopyr (e.g., fluchloraminopyr-tefuryl), fluroxypyr, fluroxypyr-MHE, halauxifen (such as halauxifen-methyl), indolauxipyr (such as indolauxipyr-cyanomethyl), mecoprop, mecoprop-P, MCPA, MCPA-thioethyl, MCPB, picloram, quinclorac, quinmerac, triclopyr, agriculturally acceptable salts and esters thereof, and combinations thereof. In some aspects, the synthetic auxin herbicide can comprise 2,4-D, MCPA, aminopyralid, clopyralid, dicamba, florpyrauxifen, fluroxypyr, halauxifen, quinclorac, agriculturally acceptable salts and esters thereof, and combinations thereof.

The synthetic auxin herbicide or agriculturally acceptable salt or ester thereof can be applied to vegetation or an area adjacent the vegetation or applied to soil or water to prevent the emergence or growth of vegetation in an amount sufficient to induce a herbicidal effect. In some aspects, the synthetic auxin herbicide or agriculturally acceptable salt or ester thereof is applied to vegetation or an area adjacent the vegetation or applied to soil or water to prevent the emergence or growth of vegetation in an amount of 1 gram acid equivalent per hectare (g ai) or more, such as 1.5 g ai or more, 2 g ai or more, 2.5 g ai or more, 3 g ai or more, 3.5 g ai or more, 4 g ai or more, 4.5 g ai or more, 5 g ai or more, 6 g ai or more, 7 g ai or more, 8 g ai or more, 9 g ai or more, 10 g ai or more, 15 g ai or more, 20 g ai or more, 25 g ai or more, 30 g ai or more, 35 g ai or more, 40 g ai or more, 45 g ai or more, 50 g ai or more, 55 g ai or more, 60 g ai or more, 65 g ai or more, 70 g ai or more, 75 g ai or more, 80 g ai or more, 85 g ai or more, 90 g ai or more, 95 g ai or more, 100 g ai or more, 110 g ai or more, 120 g ai or more, 130 g ai or more, 140 g ai or more, 150 g ai or more, 160 g ai or more, 170 g ai or more, 180 g ai or more, 190 g ai or more, 200 g ai or more, 220 g ai or more, 240 g ai or more, 250 g ai or more, 260 g ai or more, 280 g ai or more, 300 g ai or more, 320 g ai or more, 340 g ai or more, 350 g ai or more, 360 g ai or more, 380 g ai or more, 400 g ai or more, 450 g ai or more, 500 g ai or more, 550 g ai or more, 600 g ai or more, 650 g ai or more, 700 g ai or more, 750 g ai or more, 800 g ai or more, 850 g ai or more, 900 g ai or more, 950 g ai or more, 1000 g ai or more, 1050 g ai or more, 1100 g ai or more, 1150 g ai or more, 1200 g ai or more, 1250 g ai or more, 1300 g ai or more, 1350 g ai or more, 1400 g ai or more, 1450 g ai or more, 1500 g ai or more, 1600 g ai or more, 1700 g ai or more, 1800 g ai or more, 1900 g ai or more, 2000 g ai or more, 2050 g ai or more, 2100 g ai or more, 2150 g ai or more, 2200 g ai or more, 2240 g ai or more, 2250 g ai or more, 2300 g ai or more, 2350 g ai or more, 2400 g ai or more, 2450 g ai or more, 2500 g ai or more, 2600 g ai or more, 2700 g ai or more, 2750 g ai or more, 2800 g ai or more, 2900 g ai or more, 3000 g ai or more, 3050 g ai or more, 3100 g ai or more, 3150 g ai or more, 3200 g ai or more, 3250 g ai or more, 3300 g ai or more, 3350 g ai or more, 3400 g ai or more, 3450 g ai or more, 3500 g ai or more, 3600 g ai or more, 3700 g ai or more, 3750 g ai or more, 3800 g ai or more, 3900 g ai or more, or 3950 g ai or more; in an amount of 4000 g ai or less, such as 3950 g ai or less, 3900 g ai or less, 3800 g ai or less, 3750 g ai or less, 3700 g ai or less, 3600 g ai or less, 3500 g ai or less, 3450 g ai or less, 3400 g ai or less, 3350 g ai or less, 3300 g ai or less, 3250 g ai or less, 3200 g ai or less, 3150 g ai or less, 3100 g ai or less, 3050 g ai or less, 3000 g ai or less, 2900 g ai or less, 2800 g ai or less, 2750 g ai or less, 2700 g ai or less, 2600 g ai or less, 2500 g ai or less, 2450 g ai or less, 2400 g ai or less, 2350 g ai or less, 2300 g ai or less, 2250 g ai or less, 2240 g ai or less, 2200 g ai or less, 2150 g ai or less, 2100 g ai or less, 2050 g ai or less, 2000 g ai or less, 1900 g ai or less, 1800 g ai or less, 1750 g ai or less, 1700 g ai or less, 1600 g ai or less, 1500 g ai or less, 1450 g ai or less, 1400 g ai or less, 1350 g ai or less, 1300 g ai or less, 1250 g ai or less, 1240 g ai or less, 1200 g ai or less, 1150 g ai or less, 1100 g ai or less, 1050 g ai or less, 1000 g ai or less, 950 g ai or less, 900 g ai or less, 850 g ai or less, 800 g ai or less, 750 g ai or less, 700 g ai or less, 650 g ai or less, 600 g ai or less, 550 g ai or less, 500 g ai or less, 450 g ai or less, 400 g ai or less, 380 g ai or less, 360 g ai or less, 350 g ai or less, 340 g ai or less, 320 g ai or less, 300 g ai or less, 280 g ai or less, 260 g ai or less, 250 g ai or less, 240 g ai or less, 220 g ai or less, 200 g ai or less, 190 g ai or less, 180 g ai or less, 170 g ai or less, 160 g ai or less, 150 g ai or less, 140 g ai or less, 130 g ai or less, 120 g ai or less, 110 g ai or less, 100 g ai or less, 95 g ai or less, 90 g ai or less, 85 g ai or less, 80 g ai or less, 75 g ai or less, 70 g ai or less, 65 g ai or less, 60 g ai or less, 55 g ai or less, 50 g ai or less, 45 g ai or less, 40 g ai or less, 35 g ai or less, 30 g ai or less, 25 g ai or less, 20 g ai or less, 15 g ai or less, 10 g ai or less, 9 g ai or less, 8 g ai or less, 7 g ai or less, 6 g ai or less, 5 g ai or less, 4.5 g ai or less, 4 g ai or less, 3.5 g ai or less, 3 g ai or less, 2.5 g ai or less, 2 g ai or less, 1.5 g ai or less, or 1 g ai or less; or in an amount ranging from any of the minimum values described above to any of the maximum values described above, such as 1-4000 g ai, 1-2240 g ai, 1-150 g ai, 1.5-3150 g ai, 2-900 g ai, 2.5-3200 g ai, 3-1250 g ai, 5-260 g ai, 6-750 g ai, 7-2100 g ai, 10-2240 g ai, 20-3600 g ai, 40-3950 g ai, 50-400 g ai, 70-1250 g ai, 100-1400 g ai, or 250-1700 g ai.

### Phenoxycarboxylic Acids

In some aspects, the composition contains compounds according to Formula I, and (b) 2,4-D, 2,4-DB2, clomeprop, dichlorprop, mecoprop, MCPA, MCPB, or agriculturally acceptable salts or esters thereof.

### 2,4-D

In some aspects, the synthetic auxin herbicide can comprise 2,4-D or an agriculturally acceptable salt or ester thereof. 2,4-D, shown below, is a phenoxycarboxylic acid herbicide that provides broad spectrum control of many annual, biannual and perennial broad-leaved weeds and aquatic broad-leaved weeds in cereals, maize, sorghum, grasslands, established turf, grass seed crops, orchards (pome fruit and stone fruit), cranberries, asparagus, sugar cane, rice, forestry, and on non-crop land (including areas adjacent to water). 2,4-D, as well as methods of preparing 2,4-D, are known in the art. Its herbicidal activity is described, for example, in The Pesticide Manual, Seventeenth Edition, 2016.

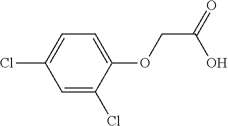 [SMILES: O=C(O)COc1ccc(Cl)cc1Cl]

In some aspects, 2,4-D can be provided as an agriculturally acceptable salt or ester of 2,4-D. Exemplary agriculturally acceptable salts and esters of 2,4-D include, but are not limited to, 2,4-D-ammonium 2,4-D-butotyl, 2,4-D-2-butoxypropyl, 2,4-D-3-butoxypropyl, 2,4-D-butyl, 2,4-D choline, 2,4-D-diethylammonium, 2,4-D-dimethylammonium (2,4-D DMA), 2,4-D-diolamine, 2,4-D-dodecylammonium, 2,4-D-ethyl, 2,4-D-2-ethylhexyl (2,4-D EHE), 2,4-D-heptylammonium, 2,4-D-isobutyl, 2,4-D-isoctyl, 2,4-D-isopropyl, 2,4-D-isopropylammonium, 2,4-D-lithium, 2,4-D-meptyl, 2,4-D-methyl, 2,4-D-octyl, 2,4-D-pentyl, 2,4-D-propyl, 2,4-D-sodium, 2,4-D-tefuryl, 2,4-D-tetradecylammonium, 2,4-D-triethylammonium, 2,4-D-tris(2-hydroxypropyl) ammonium, 2,4-D-trolamine, and clacyfos. In some aspects, the 2,4-D can be provided as 2,4-D-2-ethylhexyl (2,4-D EHE), shown below.

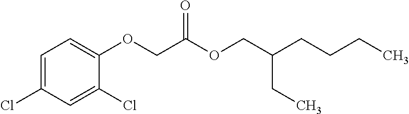 [SMILES: CCCCC(CC)COC(=O)COc1ccc(Cl)cc1Cl]

In some aspects, 2,4-D can be provided as 2,4-D DMA, shown below.

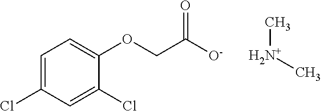

In some aspects, the 2,4-D can be provided as 2,4-D choline, shown below.

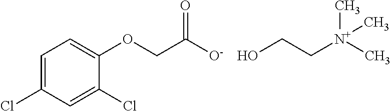

In some aspects, the 2,4-D can be provided as 2,4-D triethanolamine (2,4-D-trolamine), shown below.

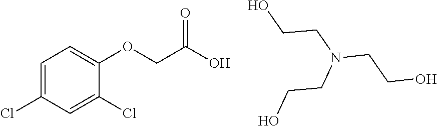 [SMILES: O=C(O)COc1ccc(Cl)cc1Cl ; OCCN(CCO)CCO]

Exemplary uses of 2,4-D-choline include, but are not limited to, controlling annual and perennial broadleaf weeds, including, but not limited to, glyphosate-resistant broadleaf weeds. 2,4-D-Choline can be used in crops that have been made tolerant to 2,4-D, including, but not limited to, 2,4-D-tolerant soybeans, maize, and cotton. 2,4-D-Choline is generally, but is not required to be, applied post-emergent. 2,4-D-Choline can also be used for weed control in non-crop and perennial cropping systems.

2,4-D can be applied to vegetation or an area adjacent the vegetation or applied to soil or water to prevent the emergence or growth of vegetation in an amount sufficient to induce a herbicidal effect. In some aspects, 2,4-D is applied to vegetation or an area adjacent the vegetation or applied to soil or water to prevent the emergence or growth of vegetation in an amount of 1 gram active ingredient per hectare (g ai/ha) or more, such as 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2240 g ai/ha or more, 2250 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3850 g ai/ha or more, 3900 g ai/ha or more, or 3950 g ai/ha or more; in an amount of 4000 g ai/ha or less, such as 3950 g ai/ha or less, 3900 g ai/ha or less, 3850 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2250 g ai/ha or less, 2240 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, 125 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, or 2 g ai/ha or less; or in an amount ranging from any of the minimum values described above to any of the maximum values described above, such as 1-4000 g ai/ha, 1-150 g ai/ha, 2-3100 g ai/ha, 2-900 g ai/ha, 3-2600 g ai/ha, 3-1200 g ai/ha, 5-275 g ai/ha, 6-750 g ai/ha, 7-2100 g ai/ha, 10-2240 g ai/ha, 20-3300 g ai/ha, 40-3950 g ai/ha, 50-400 g ai/ha, 70-2400 g ai/ha, 100-950 g ai/ha, or 250-1700 g ai/ha.

### 2,4-DB

In some aspects, the synthetic auxin herbicide can comprise 2,4-DB is a phenoxycarboxylic acid herbicide that provides post-emergence control of many annual and perennial broadleaf weeds in alfalfa, clovers, cereals, grassland, forage, legumes, soybeans, and peanuts. 2,4-DB, as well as methods of making 2,4-DB, are known in the art. Its herbicidal activity is described in The Pesticide Manual, Seventeenth Edition, 2016.

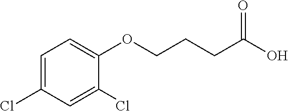 [SMILES: O=C(O)CCCOc1ccc(Cl)cc1Cl]

In some aspects, 2,4-DB can be provided as an agriculturally acceptable salt or ester of 2,4-DB. Exemplary agriculturally acceptable salts and esters of 2,4-DB include, but are not limited to, 2,4-DB-butyl, 2,4-DB-dimethylammonium (2,4-DB DMA), 2,4-DB-isoctyl, 2,4-DB-potassium, and 2,4-DB-sodium.

2,4-DB can be applied to vegetation or an area adjacent the vegetation or applied to soil or water to prevent the emergence or growth of vegetation in an amount sufficient to induce a herbicidal effect. In some aspects, 2,4-DB is applied to vegetation or an area adjacent the vegetation or applied to soil or water to prevent the emergence or growth of vegetation in an amount of 1 g ai/ha or more, such as 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2240 g ai/ha or more, 2250 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3850 g ai/ha or more, 3900 g ai/ha or more, or 3950 g ai/ha or more; in an amount of 4000 g ai/ha or less, such as 3950 g ai/ha or less, 3900 g ai/ha or less, 3850 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2250 g ai/ha or less, 2240 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, 125 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, or 2 g ai/ha or less; or in an amount ranging from any of the minimum values described above to any of the maximum values described above, such as 1-4000 g ai/ha, 1-150 g ai/ha, 2-3100 g ai/ha, 2-900 g ai/ha, 3-2600 g ai/ha, 3-1200 g ai/ha, 5-275 g ai/ha, 6-750 g ai/ha, 7-2100 g ai/ha, 10-2240 g ai/ha, 20-3300 g ai/ha, 40-3950 g ai/ha, 50-400 g ai/ha, 70-2400 g ai/ha, 100-950 g ai/ha, or 250-1700 g ai/ha.

### Clomeprop

In some aspects, the synthetic auxin herbicide can comprise clomeprop or an agriculturally acceptable salt or ester thereof. Clomeprop, shown below, is a phenoxycarboxylic acid herbicide that provides pre- to early post-emergence control of broad-leaf and sedge weeds in paddy rice. Clomeprop, as well as methods of preparing clomeprop, are known in the art. Its herbicidal activity is described, for example, in The Pesticide Manual, Seventeenth Edition, 2016.

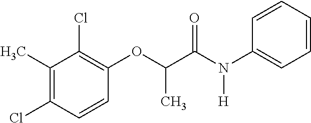 [SMILES: Cc1c(Cl)ccc(OC(C)C(=O)Nc2ccccc2)c1Cl]

### Dichlorprop

In some aspects, the synthetic auxin herbicide can comprise dichlorprop or an agriculturally acceptable salt or ester thereof. Dichlorprop, shown below, is a phenoxycarboxylic herbicide that provides broad-spectrum control of annual and perennial broad-leaved weeds in cereals and grasslands; brush control in non-crop land; control of broad-leaved aquatic weeds; and maintenance of embankments and roadside verges. Dichlorprop, as well as methods of making dichlorprop, are known in the art. Its herbicidal activity is described, for example, in The Pesticide Manual, Seventeenth Edition, 2016.

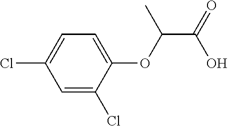

In some aspects, dichlorprop can be provided as an agriculturally acceptable salt or ester of dichlorprop. Exemplary agriculturally acceptable salts and esters of dichlorprop include, but are not limited to, dichlorprop-butotyl, dichlorprop-dimethylammonium, dichlorprop-ethylammonium, dichlorprop-2-ethylhexyl, dichlorprop-isoctyl, dichlorprop-methyl, dichlorprop-potassium, dichlorprop-sodium, dichlorprop-P, dichlorprop-P-dimethylammonium, dichlorprop-P-2-ethylhexyl, dichlorprop-P-potassium, and dichlorprop-P-sodium. In some aspects, the dichlorprop can be provided as the stereoisomer dichlorprop-P, shown below.

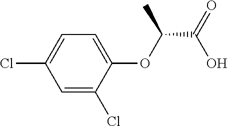

Dichlorprop can be applied to vegetation or an area adjacent the vegetation or applied to soil or water to prevent the emergence or growth of vegetation in an amount sufficient to induce a herbicidal effect. In some aspects, dichlorprop is applied to vegetation or an area adjacent the vegetation or applied to soil or water to prevent the emergence or growth of vegetation in an amount of 1 g ai/ha or more, such as 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2240 g ai/ha or more, 2250 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3850 g ai/ha or more, 3900 g ai/ha or more, or 3950 g ai/ha or more; in an amount of 4000 g ai/ha or less, such as 3950 g ai/ha or less, 3900 g ai/ha or less, 3850 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2250 g ai/ha or less, 2240 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, 125 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, or 2 g ai/ha or less; or in an amount ranging from any of the minimum values described above to any of the maximum values described above, such as 1-4000 g ai/ha, 1-150 g ai/ha, 2-3100 g ai/ha, 2-900 g ai/ha, 3-2600 g ai/ha, 3-1200 g ai/ha, 5-275 g ai/ha, 6-750 g ai/ha, 7-2100 g ai/ha, 10-2240 g ai/ha, 20-3300 g ai/ha, 40-3950 g ai/ha, 50-400 g ai/ha, 70-2400 g ai/ha, 100-950 g ai/ha, or 250-1700 g ai/ha.

### Mecoprop

In some aspects, the synthetic auxin herbicide can comprise mecoprop or an agriculturally acceptable salt or ester thereof. Mecoprop, shown below, is a phenoxy carboxylic acid herbicide that provides broad-spectrum control of broad-leaved weeds in wheat, barley, oats, herbage seed crops, grassland, and under fruit trees and vines. Mecoprop also provides control of docks (Rumex spp.) in meadows and pastures. Mecoprop, as well as methods of making mecoprop, are known in the art. Its herbicidal activity is described in The Pesticide Manual, Seventeenth Edition, 2016.

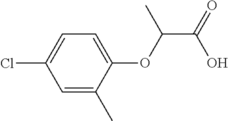

In some aspects, mecoprop can be provided as an agriculturally acceptable salt or ester of mecoprop. Exemplary agriculturally acceptable salts and esters of mecoprop include, but are not limited to, mecoprop-butotyl, mecoprop-potassium, mecoprop-sodium, mecoprop-dimethylammonium, mecoprop-diolamine, mecoprop-ethadyl, mecoprop-2-ethylhexyl, mecoprop-isoctyl, mecoprop-methyl, mecoprop-trolamine, mecoprop-P, mecoprop-P-butotyl, mecoprop-P-dimethylammonium, mecoprop-P-ethylhexyl, and mecoprop-P-potassium. In some aspects, the mecoprop can be provided as the stereoisomer mecoprop-P, shown below.

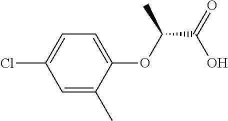

Mecoprop can be applied to vegetation or an area adjacent the vegetation or applied to soil or water to prevent the emergence or growth of vegetation in an amount sufficient to induce a herbicidal effect. In some aspects, mecoprop is applied to vegetation or an area adjacent the vegetation or applied to soil or water to prevent the emergence or growth of vegetation in an amount of 1 g ai/ha or more, such as 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2240 g ai/ha or more, 2250 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3850 g ai/ha or more, 3900 g ai/ha or more, or 3950 g ai/ha or more; in an amount of 4000 g ai/ha or less, such as 3950 g ai/ha or less, 3900 g ai/ha or less, 3850 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2250 g ai/ha or less, 2240 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, 125 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, or 2 g ai/ha or less; or in an amount ranging from any of the minimum values described above to any of the maximum values described above, such as 1-4000 g ai/ha, 1-150 g ai/ha, 2-3100 g ai/ha, 2-900 g ai/ha, 3-2600 g ai/ha, 3-1200 g ai/ha, 5-275 g ai/ha, 6-750 g ai/ha, 7-2100 g ai/ha, 10-2240 g ai/ha, 20-3300 g ai/ha, 40-3950 g ai/ha, 50-400 g ai/ha, 70-2400 g ai/ha, 100-950 g ai/ha, or 250-1700 g ai/ha.

### MCPA

In some aspects, the synthetic auxin herbicide can comprise MCPA or an agriculturally acceptable salt or ester thereof. MCPA, shown below, is a phenoxycarboxylic acid herbicide that provides broad-spectrum control of many annual, biannual, and perennial broad-leaved weeds, woody weeds, and aquatic broad-leaved weeds in cereals, herbage seed crops, flax, rice, vines, peas, potatoes, asparagus, grassland, turf, under fruit trees, forestry, and on roadside verges and embankments. MCPA, as well as methods of preparing MCPA, are known in the art. Its herbicidal activity is described, for example, in The Pesticide Manual, Seventeenth Edition, 2016.

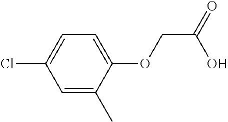

In some aspects, MCPA can be provided as an agriculturally acceptable salt or ester of MCPA. Exemplary agriculturally acceptable salts of MCPA include, but are not limited to, MCPA-butotyl, MCPA-butyl, MCPA-dimethylammonium (MCPA-DMA), MCPA-diolamine, MCPA-ethyl, MCPA-2-ethylhexyl (MCPA EHE), MCPA-isobutyl, MCPA-isoctyl, MCPA-isopropyl, MCPA-methyl, MCPA-olamine, MCPA-potassium, MCPA-sodium, MCPA-trolamine. In some aspects, the MCPA can be provided as MCPA-2-ethylhexyl (MCPA EHE), shown below.

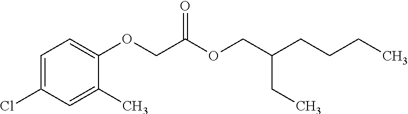 [SMILES: CCCCC(CC)COC(=O)COc1ccc(Cl)cc1C]

In some aspects, the MCPA can be provided as MCPA-dimethylammonium (MCPA-DMA), shown below.

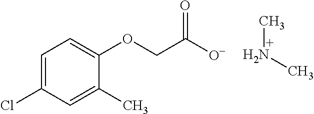

MCPA can be applied to vegetation or an area adjacent the vegetation or applied to soil or water to prevent the emergence or growth of vegetation in an amount sufficient to induce a herbicidal effect. In some aspects, MCPA is applied to vegetation or an area adjacent the vegetation or applied to soil or water to prevent the emergence or growth of vegetation in an amount of 1 g ai/ha or more such as 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2240 g ai/ha or more, 2250 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3850 g ai/ha or more, 3900 g ai/ha or more, or 3950 g ai/ha or more; in an amount of 4000 g ai/ha or less, such as 3950 g ai/ha or less, 3900 g ai/ha or less, 3850 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2250 g ai/ha or less, 2240 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, 125 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, or 2 g ai/ha or less; or in an amount ranging from any of the minimum values described above to any of the maximum values described above, such as 1-4000 g ai/ha, 1-150 g ai/ha, 2-3100 g ai/ha, 2-900 g ai/ha, 3-2600 g ai/ha, 3-1200 g ai/ha, 5-275 g ai/ha, 6-750 g ai/ha, 7-2100 g ai/ha, 10-2240 g ai/ha, 20-3300 g ai/ha, 40-3950 g ai/ha, 50-400 g ai/ha, 70-2400 g ai/ha, 100-950 g ai/ha, or 250-1700 g ai/ha.

### MCPB

In some aspects, the synthetic auxin herbicide can comprise MCPB or an agriculturally acceptable salt or ester thereof. MCPB, shown below, is a phenoxycarboxylic acid herbicide that provides broad-spectrum control of many broad-leaved weeds, woody weeds, and aquatic broad-leaved weeds in rice, cereals, clovers, sainfoin, peas, peanuts, grassland, turf, and forestry. MCPB, as well as methods of preparing MCPB, are known in the art. Its herbicidal activity is described, for example, in The Pesticide Manual, Seventeenth Edition, 2016.

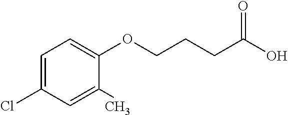

In some aspects, MCPB can be provided as an agriculturally acceptable salt or ester of MCPB. Exemplary agriculturally acceptable salts of MCPB include, but are not limited to, MCPB-ethyl, MCPB-potassium, and MCPB-sodium.

MCPB can be applied to vegetation or an area adjacent the vegetation or applied to soil or water to prevent the emergence or growth of vegetation in an amount sufficient to induce a herbicidal effect. In some aspects, MCPB is applied to vegetation or an area adjacent the vegetation or applied to soil or water to prevent the emergence or growth of vegetation in an amount of 1 g ai/ha or more, such as 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2240 g ai/ha or more, 2250 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3850 g ai/ha or more, 3900 g ai/ha or more, or 3950 g ai/ha or more; in an amount of 4000 g ai/ha or less, such as 3950 g ai/ha or less, 3900 g ai/ha or less, 3850 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2250 g ai/ha or less, 2240 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, 125 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, or 2 g ai/ha or less; or in an amount ranging from any of the minimum values described above to any of the maximum values described above, such as 1-4000 g ai/ha, 1-150 g ai/ha, 2-3100 g ai/ha, 2-900 g ai/ha, 3-2600 g ai/ha, 3-1200 g ai/ha, 5-275 g ai/ha, 6-750 g ai/ha, 7-2100 g ai/ha, 10-2240 g ai/ha, 20-3300 g ai/ha, 40-3950 g ai/ha, 50-400 g ai/ha, 70-2400 g ai/ha, 100-950 g ai/ha, or 250-1700 g ai/ha.

### Benzoic Acids

In some aspects, the composition contains compounds according to Formula I, and (b) chloramben, dicamba, TBA, or agriculturally acceptable salts or esters thereof.

### Chloramben

In some aspects, the synthetic auxin herbicide can comprise chloramben or an agriculturally acceptable salt or ester thereof. Chloramben, shown below, is a benzoic acid herbicide that provides pre-planting and pre-emergence control of grasses and broad-leaved weeds in asparagus, navy beans, peanuts, maize, sweet potatoes, pumpkins, soybeans, squash, sunflowers, and certain ornamentals. Chloramben, as well as methods of preparing chloramben, are known in the art. Its herbicidal activity is described, for example, in The Pesticide Manual, Seventeenth Edition, 2016.

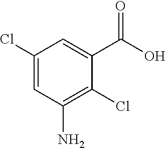 [SMILES: Nc1cc(Cl)cc(C(=O)O)c1Cl]

### Dicamba

In some aspects, the synthetic auxin herbicide can comprise dicamba or an agriculturally acceptable salt or ester thereof. Dicamba, shown below, is a benzoic acid herbicide that provides broad-spectrum control of annual and perennial broad-leaved weeds and brush species in cereals, maize, sorghum, sugar cane, asparagus, perennial seed grasses, turf, pastures, rangeland, and non-crop land. Dicamba, as well as methods of making dicamba, are known in the art. Its herbicidal activity is described, for example, in The Pesticide Manual, Seventeenth Edition, 2016.

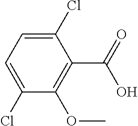

In some aspects, dicamba can be provided as an agriculturally acceptable salt or ester of dicamba. Exemplary agriculturally acceptable salts and esters of dicamba include, but are not limited to, dicamba-diglycolammonium, dicamba-dimethylammonium, dicamba-diolamine, dicamba-isopropylammonium, dicamba-methyl, dicamba-olamine, dicamba-potassium, dicamba-sodium, dicamba-trolamine, and cambendichlor.

In some aspects, dicamba can be provided as dicamba-diglycolammonium, shown below.

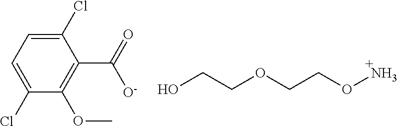

Dicamba can be applied to vegetation or an area adjacent the vegetation or applied to soil or water to prevent the emergence or growth of vegetation in an amount sufficient to induce a herbicidal effect. In some aspects, dicamba is applied to vegetation or an area adjacent the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 425 g ai/ha or more, 450 g ai/ha or more, 475 g ai/ha or more, 500 g ai/ha or more, 525 g ai/ha or more, 550 g ai/ha or more, 560 g ai/ha or more, 570 g ai/ha or more, 575 g ai/ha or more, 580 g ai/ha or more, 590 g ai/ha or more, or 600 g ai/ha or more; in an amount of 600 g ai/ha or less such as 590 g ai/ha or less, 580 g ai/ha or less, 575 g ai/ha or less, 570 g ai/ha or less, 560 g ai/ha or less, 550 g ai/ha or less, 525 g ai/ha or less, 500 g ai/ha or less, 475 g ai/ha or less, 450 g ai/ha or less, 425 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-600 g ai/ha, 55-150 g ai/ha, 60-310 g ai/ha, 65-190 g ai/ha, 80-560 g ai/ha, 85-300 g ai/ha, 100-275 g ai/ha, 120-475 g ai/ha, 140-390 g ai/ha, 150-250 g ai/ha, or 250-400 g ai/ha. 2,3,6-TBA

In some aspects, the synthetic auxin herbicide can comprise 2,3,6-TBA or an agriculturally acceptable salt or ester thereof. 2,3,6-TBA, shown below, is a benzoic acid herbicide that provides post-emergence control of certain broad-leaved annual and perennial weeds in cereals and grass seed crops. 2,3,6-TBA, as well as methods of preparing 2,3,6-TBA, are known in the art. Its herbicidal activity is described, for example, in The Pesticide Manual, Seventeenth Edition, 2016.

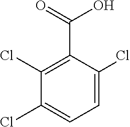 [SMILES: O=C(O)c1c(Cl)ccc(Cl)c1Cl]

### C. Aryl Picolinates

In some aspects, the composition contains compounds according to Formula I, and (b) florpyrauxifen, halauxifen, or agriculturally acceptable salts or esters thereof.

### Florpyrauxifen

In some aspects, the synthetic auxin herbicide can comprise florpyrauxifen or an agriculturally acceptable salt or ester thereof. Florpyrauxifen is aryl picolinic acid that has been described in U.S. Pat. No. 7,314,849 B2. Exemplary uses of florpyrauxifen include controlling undesirable vegetation, including grass, broadleaf and sedge weeds, in multiple non-crop and cropping situations.

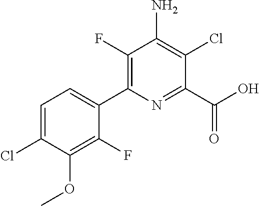 [SMILES: COc1c(Cl)ccc(-c2nc(C(=O)O)c(Cl)c(N)c2F)c1F]

In some aspects, florpyrauxifen can be provided as an agriculturally acceptable salt or ester of florpyrauxifen. Exemplary agriculturally acceptable salts and esters of florpyrauxifen include, but are not limited to, florpyrauxifen-benzyl, shown below.

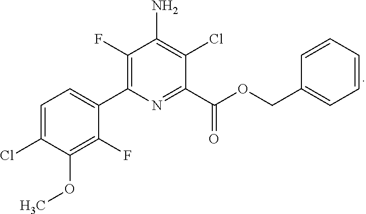 [SMILES: COc1c(Cl)ccc(-c2nc(C(=O)OCc3ccccc3)c(Cl)c(N)c2F)c1F]

Florpyrauxifen can be applied to vegetation or an area adjacent the vegetation or applied to soil or water to prevent the emergence or growth of vegetation in an amount sufficient to induce a herbicidal effect. In some aspects, florpyrauxifen is applied to vegetation or an area adjacent the vegetation or applied to soil or water to prevent the emergence or growth of vegetation in an amount of 1 g ai/ha or more, such as 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 110 g ai/ha or more, 120 g ai/ha or more, 125 g ai/ha or more, 130 g ai/ha or more, 140 g ai/ha or more, 142.5 g ai/ha or more, 145 g ai/ha or more, 147.5 g ai/ha or more, or 149 g ai/ha or more; in an amount of 150 g ai/ha or less, such as 149 g ai/ha or less, 147.5 g ai/ha or less, 145 g ai/ha or less, 142.5 g ai/ha or less, 140 g ai/ha or less, 130 g ai/ha or less, 125 g ai/ha or less, 120 g ai/ha or less, 110 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, or 2 g ai/ha or less; or in an amount ranging from any of the minimum values described above to any of the maximum values described above, such as 1-150 g ai/ha, 2-130 g ai/ha, 2-45 g ai/ha, 3-70 g ai/ha, 4-147.5 g ai/ha, 5-100 g ai/ha, 6-75 g ai/ha, 7-50 g ai/ha, 10-149 g ai/ha, 20-120 g ai/ha, 25-90 g ai/ha, 30-60 g ai/ha, 50-150 g ai/ha, 60-130 g ai/ha, or 100-145 g ai/ha.

### Halauxifen

In some aspects, the synthetic auxin herbicide can comprise halauxifen or an agriculturally acceptable salt or ester thereof. Halauxifen, shown below, is a aryl picolinic acid herbicide that provides broad-spectrum control of broad-leaved weeds in cereal crops.

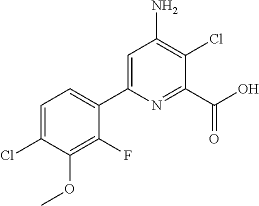 [SMILES: COc1c(Cl)ccc(-c2cc(N)c(Cl)c(C(=O)O)n2)c1F]

In some aspects, halauxifen can be provided as an agriculturally acceptable salt or ester of halauxifen. Exemplary agriculturally acceptable salts and esters of halauxifen include, but are not limited to, halauxifen-methyl. In some aspects, the halauxifen is provided as halauxifen-methyl, shown below.

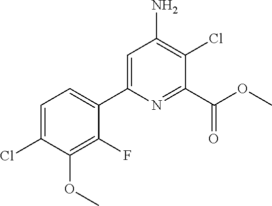 [SMILES: COC(=O)c1nc(-c2ccc(Cl)c(OC)c2F)cc(N)c1Cl]

Halauxifen can be applied to vegetation or an area adjacent the vegetation or applied to soil or water to prevent the emergence or growth of vegetation in an amount sufficient to induce a herbicidal effect. In some aspects, halauxifen is applied to vegetation or an area adjacent the vegetation or applied to soil or water to prevent the emergence or growth of vegetation in an amount of 1 g ai/ha or more, such as 2 g ai/ha or more, 2.5 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 110 g ai/ha or more, 120 g ai/ha or more, 125 g ai/ha or more, 130 g ai/ha or more, 140 g ai/ha or more, 142.5 g ai/ha or more, 145 g ai/ha or more, 147.5 g ai/ha or more, or 149 g ai/ha or more; in an amount of 150 g ai/ha or less, such as 149 g ai/ha or less, 147.5 g ai/ha or less, 145 g ai/ha or less, 142.5 g ai/ha or less, 140 g ai/ha or less, 130 g ai/ha or less, 125 g ai/ha or less, 120 g ai/ha or less, 110 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, 2.5 g ai/ha or less, or 2 g ai/ha or less; or in an amount ranging from any of the minimum values described above to any of the maximum values described above, such as 1-150 g ai/ha, 2-130 g ai/ha, 2.5-45 g ai/ha, 3-70 g ai/ha, 4-147.5 g ai/ha, 5-100 g ai/ha, 6-75 g ai/ha, 7-50 g ai/ha, 10-149 g ai/ha, 20-120 g ai/ha, 25-90 g ai/ha, 30-60 g ai/ha, 50-150 g ai/ha, 60-130 g ai/ha, or 100-145 g ai/ha.

### Pyridine Carboxylates

In some aspects, the composition contains compounds according to Formula I, and (b) aminopyralid, clopyralid, fluroxypyr, picloram, triclopyr, or agriculturally acceptable salts or esters thereof.

### Aminopyralid

In some aspects, the synthetic auxin herbicide can comprise aminopyralid or an agriculturally acceptable salt or ester thereof. Aminopyralid is a compounds according to Formula I that provides pre- and post-emergence control of certain annual and perennial broadleaf weeds in grasslands. Aminopyralid, as well as methods of making aminopyralid, are known in the art. Its herbicidal activity is described in The Pesticide Manual, Seventeenth Edition, 2016.

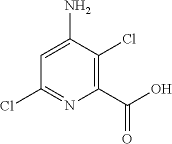 [SMILES: Nc1cc(Cl)nc(C(=O)O)c1Cl]

In some aspects, aminopyralid can be provided as an agriculturally acceptable salt or ester of aminopyralid. Exemplary agriculturally acceptable salts and esters of aminopyralid include, but are not limited to, for example, aminopyralid-triisopropanolammonium (TIPA), shown below.

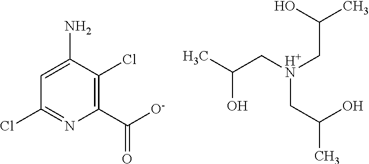

Aminopyralid can be applied to vegetation or an area adjacent the vegetation or applied to soil or water to prevent the emergence or growth of vegetation in an amount sufficient to induce a herbicidal effect. In some aspects, aminopyralid is applied to vegetation or an area adjacent the vegetation or applied to soil or water to prevent the emergence or growth of vegetation in an amount of 1 g ai/ha or more, such as 2 g ai/ha or more, 2.5 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 110 g ai/ha or more, 120 g ai/ha or more, 125 g ai/ha or more, 130 g ai/ha or more, 140 g ai/ha or more, 142.5 g ai/ha or more, 145 g ai/ha or more, 147.5 g ai/ha or more, or 149 g ai/ha or more; in an amount of 150 g ai/ha or less, such as 149 g ai/ha or less, 147.5 g ai/ha or less, 145 g ai/ha or less, 142.5 g ai/ha or less, 140 g ai/ha or less, 130 g ai/ha or less, 125 g ai/ha or less, 120 g ai/ha or less, 110 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, 2.5 g ai/ha or less, or 2 g ai/ha or less; or in an amount ranging from any of the minimum values described above to any of the maximum values described above, such as 1-150 g ai/ha, 2-130 g ai/ha, 2.5-45 g ai/ha, 3-70 g ai/ha, 4-147.5 g ai/ha, 5-100 g ai/ha, 6-75 g ai/ha, 7-50 g ai/ha, 10-149 g ai/ha, 20-120 g ai/ha, 25-90 g ai/ha, 30-60 g ai/ha, 50-150 g ai/ha, 60-130 g ai/ha, or 100-145 g ai/ha.

### Clopyralid

In some aspects, the synthetic auxin herbicide can comprise clopyralid or an agriculturally acceptable salt or ester thereof. Clopyralid, shown below, is a compounds according to Formula I that provides post-emergence control of many annual and perennial broad-leaved weeds in sugar beet, fodder beet, oilseed rape, maize, cereals, brassicas, onions, leeks, strawberries, flax, grassland, and non-crop lands. Clopyralid, as well as methods of making clopyralid, are known in the art. Its herbicidal activity is described, for example, in The Pesticide Manual, Seventeenth Edition, 2016.

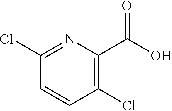 [SMILES: O=C(O)c1nc(Cl)ccc1Cl]

In some aspects, clopyralid can be provided as an agriculturally acceptable salt or ester of clopyralid. Exemplary agriculturally acceptable salts and esters of clopyralid include, but are not limited to, clopyralid-methyl, clopyralid-potassium, clopyralid-tris(2-hydroxypropyl) ammonium, and clopyralid-olamine, shown below.

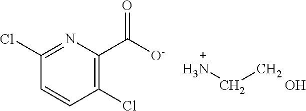

Clopyralid can be applied to vegetation or an area adjacent the vegetation or applied to soil or water to prevent the emergence or growth of vegetation in an amount sufficient to induce a herbicidal effect. In some aspects, clopyralid is applied to vegetation or an area adjacent the vegetation or applied to soil or water to prevent the emergence or growth of vegetation in an amount of 25 g ai/ha or more, such as 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, or 390 g ai/ha or more; in an amount of 400 g ai/ha or less, such as 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less, 50 g ai/ha or less, 45 g ai/ha or less, 40 g ai/ha or less, 35 g ai/ha or less, or 30 g ai/ha or less; or in an amount ranging from any of the minimum values described above to any of the maximum values described above, such as 25-400 g ai/ha, 55-150 g ai/ha, 60-310 g ai/ha, 65-190 g ai/ha, 80-260 g ai/ha, 85-300 g ai/ha, 100-275 g ai/ha, 120-330 g ai/ha, 140-390 g ai/ha, 150-250 g ai/ha, or 30-400 g ai/ha.

### Fluroxypyr

In some aspects, the synthetic auxin herbicide can comprise fluroxypyr or an agriculturally acceptable salt or ester thereof. Fluroxypyr, shown below, is a compounds according to Formula I that provides post-emergence control against certain broad-leaved weeds in small grain crops, pastures, grasslands, orchards, plantation crops, maize, and forestry. Fluroxypyr, as well as methods of making fluroxypyr, are known in the art. Its herbicidal activity is described, for example, in The Pesticide Manual, Seventeenth Edition, 2016.

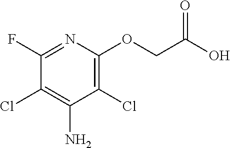 [SMILES: Nc1c(Cl)c(F)nc(OCC(=O)O)c1Cl]

In some aspects, fluroxypyr can be provided as an agriculturally acceptable salt or ester of fluroxypyr. Exemplary agriculturally acceptable salts and esters of fluroxypyr include, but are not limited to, fluroxypyr-2-butoxy-1-methylethyl (fluroxypyr-butometyl) and fluroxypyr-meptyl (fluroxypyr-MHE). In some aspects, the fluroxypyr can be provided as fluroxypyr-meptyl (fluroxypyr-MHE), shown below.

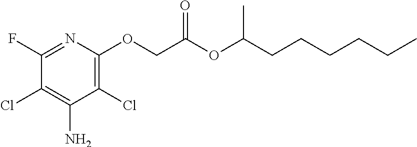 [SMILES: CCCCCCC(C)OC(=O)COc1nc(F)c(Cl)c(N)c1Cl]

Fluroxypyr can be applied to vegetation or an area adjacent the vegetation or applied to soil or water to prevent the emergence or growth of vegetation in an amount sufficient to induce a herbicidal effect. In some aspects, fluroxypyr is applied to vegetation or an area adjacent the vegetation or applied to soil or water to prevent the emergence or growth of vegetation in an amount of 35 g ai/ha or more, such as 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, or 390 g ai/ha or more; in an amount of 400 g ai/ha or less, such as 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, or 35 g ai/ha or less; or in an amount ranging from any of the minimum values described above to any of the maximum values described above, such as 35-400 g ai/ha, 55-150 g ai/ha, 60-310 g ai/ha, 45-190 g ai/ha, 80-260 g ai/ha, 85-300 g ai/ha, 100-275 g ai/ha, 120-330 g ai/ha, 140-390 g ai/ha, 150-250 g ai/ha, or 250-400 g ai/ha.

### Picloram

In some aspects, the synthetic auxin herbicide can comprise picloram or an agriculturally acceptable salt or ester thereof. Picloram is a compounds according to Formula I that provides management of unwanted vegetation in rangeland, grass pastures, forestry, as well as non-crop land and right-of-way sites. Picloram, as well as methods of making picloram, is known in the art. Its herbicidal activity is described in The Pesticide Manual, Seventeenth Edition, 2016.

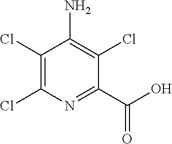 [SMILES: Nc1c(Cl)c(Cl)nc(C(=O)O)c1Cl]

In some aspects, picloram can be provided as an agriculturally acceptable salt or ester of picloram. Exemplary agriculturally acceptable salts and esters of picloram include, but are not limited to, picloram-dimethylammonium, picloram-isoctyl, picloram-potassium, picloram-triethylammonium, picloram-triisopropanolammonium (TIPA), picloram-triisopropylammonium, and picloram-trolamine (triethanolammonium). In some aspects, the picloram can be provided as picloram-potassium, shown below.

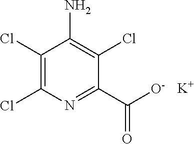 [SMILES: Nc1c(Cl)c(Cl)nc(C(=O)[O-])c1Cl ; [K+]]

In some aspects, the picloram can be provided as picloram-TIPA, shown below.

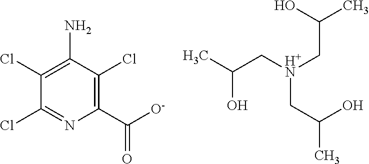

Picloram can be applied to vegetation or an area adjacent the vegetation or applied to soil or water to prevent the emergence or growth of vegetation in an amount sufficient to induce a herbicidal effect. In some aspects, picloram is applied to vegetation or an area adjacent the vegetation or applied to soil or water to prevent the emergence or growth of vegetation in an amount of 10 g ai/ha or more, such as 12 g ai/ha or more, 14 g ai/ha or more, 15 g ai/ha or more, 16 g ai/ha or more, 18 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2050 g ai/ha or more, 2100 g ai/ha or more, 2150 g ai/ha or more, 2200 g ai/ha or more, or 2220 g ai/ha or more; in an amount of 22400 g ai/ha or less, such as 2220 g ai/ha or less, 2200 g ai/ha or less, 2150 g ai/ha or less, 2100 g ai/ha or less, 2050 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, 125 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 18 g ai/ha or less, 16 g ai/ha or less, 15 g ai/ha or less, 14 g ai/ha or less, or 12 g ai/ha or less; or in an amount ranging from any of the minimum values described above to any of the maximum values described above, such as 10-2240 g ai/ha, 12-1500 g ai/ha, 14-2100 g ai/ha, 15-900 g ai/ha, 18-1600 g ai/ha, 30-1200 g ai/ha, 50-275 g ai/ha, 70-1000 g ai/ha, 100-2220 g ai/ha, 250-1300 g ai/ha, 450-2000 g ai/ha, or 500-1900 g ai/ha.

### Triclopyr

In some aspects, the synthetic auxin herbicide can comprise triclopyr or an agriculturally acceptable salt or ester thereof. Triclopyr is a compounds according to Formula I that provides control of woody plants and broadleaf weed species in grassland, rangeland, industrial areas, coniferous forests, rice, and plantation crops. Triclopyr, as well as methods of making triclopyr, are known in the art. Its herbicidal activity is described in The Pesticide Manual, Seventeenth Edition, 2016.

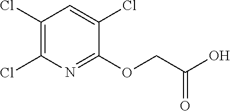 [SMILES: O=C(O)COc1nc(Cl)c(Cl)cc1Cl]

In some aspects, triclopyr can be provided as an agriculturally acceptable salt or ester of triclopyr. Exemplary agriculturally acceptable salts and esters of triclopyr include, but are not limited to, for example, triclopyr-triethylammonium (TEA), triclopyr choline, and triclopyr-butotyl (butoxyethyl or BEE). In some aspects, the triclopyr can be provided as triclopyr-triethylammonium (TEA), shown below.

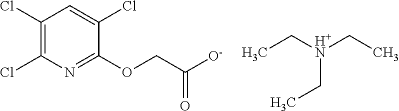

In some aspects, the triclopyr can be provided as triclopyr-choline, shown below.

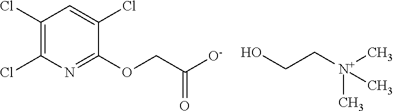

In some aspects, the triclopyr can be provided as triclopyr-BEE, shown below.

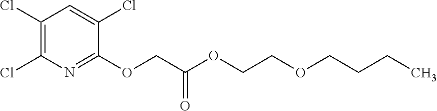 [SMILES: CCCCOCCOC(=O)COc1nc(Cl)c(Cl)cc1Cl]

Triclopyr can be applied to vegetation or an area adjacent the vegetation or applied to soil or water to prevent the emergence or growth of vegetation in an amount sufficient to induce a herbicidal effect. In some aspects, triclopyr is applied to vegetation or an area adjacent the vegetation or applied to soil or water to prevent the emergence or growth of vegetation in an amount of 10 g ai/ha or more, such as 12 g ai/ha or more, 14 g ai/ha or more, 15 g ai/ha or more, 16 g ai/ha or more, 18 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2050 g ai/ha or more, 2100 g ai/ha or more, 2150 g ai/ha or more, 2200 g ai/ha or more, or 2220 g ai/ha or more; in an amount of 22400 g ai/ha or less, such as 2220 g ai/ha or less, 2200 g ai/ha or less, 2150 g ai/ha or less, 2100 g ai/ha or less, 2050 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, 125 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 18 g ai/ha or less, 16 g ai/ha or less, 15 g ai/ha or less, 14 g ai/ha or less, or 12 g ai/ha or less; or in an amount ranging from any of the minimum values described above to any of the maximum values described above, such as 10-2240 g ai/ha, 12-1500 g ai/ha, 14-2100 g ai/ha, 15-900 g ai/ha, 18-1600 g ai/ha, 30-1200 g ai/ha, 50-275 g ai/ha, 70-1000 g ai/ha, 100-2220 g ai/ha, 250-1300 g ai/ha, 450-2000 g ai/ha, or 500-1900 g ai/ha.

### Quinoline Carboxylic Acids

In some aspects, the composition contains compounds according to Formula I, and (b) quinclorac, quinmerac, or agriculturally acceptable salts or esters thereof.

### Quinclorac

In some aspects, the synthetic auxin herbicide can comprise quinclorac or an agriculturally acceptable salt or ester thereof. Quinclorac, shown below, is a quinoline carboxylic acid herbicide that provides pre- and post-emergence control of certain grass weeds and other weeds in direct-seeded and transplanted rice. Quinclorac, as well as methods of making quinclorac, are known in the art. Its herbicidal activity is described in The Pesticide Manual, Seventeenth Edition, 2016.

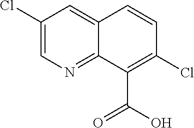 [SMILES: O=C(O)c1c(Cl)ccc2cc(Cl)cnc12]

In some aspects, quinclorac can be provided as an agriculturally acceptable salt or ester of quinclorac. Exemplary agriculturally acceptable salts and esters of quinclorac include, but are not limited to, quinclorac-dimethylammonium.

Quinclorac can be applied to vegetation or an area adjacent the vegetation or applied to soil or water to prevent the emergence or growth of vegetation in an amount sufficient to induce a herbicidal effect. In some aspects, quinclorac is applied to vegetation or an area adjacent the vegetation or applied to soil or water to prevent the emergence or growth of vegetation in an amount of 10 g ai/ha or more, such as 12 g ai/ha or more, 14 g ai/ha or more, 15 g ai/ha or more, 16 g ai/ha or more, 18 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2050 g ai/ha or more, 2100 g ai/ha or more, 2150 g ai/ha or more, 2200 g ai/ha or more, or 2220 g ai/ha or more; in an amount of 22400 g ai/ha or less, such as 2220 g ai/ha or less, 2200 g ai/ha or less, 2150 g ai/ha or less, 2100 g ai/ha or less, 2050 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, 125 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 18 g ai/ha or less, 16 g ai/ha or less, 15 g ai/ha or less, 14 g ai/ha or less, or 12 g ai/ha or less; or in an amount ranging from any of the minimum values described above to any of the maximum values described above, such as 10-2240 g ai/ha, 12-1500 g ai/ha, 14-2100 g ai/ha, 15-900 g ai/ha, 18-1600 g ai/ha, 30-1200 g ai/ha, 50-275 g ai/ha, 70-1000 g ai/ha, 100-2220 g ai/ha, 250-1300 g ai/ha, 450-2000 g ai/ha, or 500-1900 g ai/ha.

### Quinmerac

In some aspects, the synthetic auxin herbicide can comprise quinmerac or an agriculturally acceptable salt or ester thereof. Quinmerac, shown below, is a quinoline carboxylic acid herbicide that provides control of certain broad-leaved weeds in cereals, oilseed rape, and sugar beets. Quinmerac, as well as methods of making quinmerac, are known in the art. Its herbicidal activity is described in The Pesticide Manual, Seventeenth Edition, 2016.

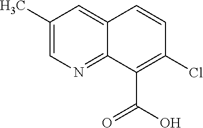

Quinmerac can be applied to vegetation or an area adjacent the vegetation or applied to soil or water to prevent the emergence or growth of vegetation in an amount sufficient to induce a herbicidal effect. In some aspects, quinmerac is applied to vegetation or an area adjacent the vegetation or applied to soil or water to prevent the emergence or growth of vegetation in an amount of 10 g ai/ha or more, such as 12 g ai/ha or more, 14 g ai/ha or more, 15 g ai/ha or more, 16 g ai/ha or more, 18 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2050 g ai/ha or more, 2100 g ai/ha or more, 2150 g ai/ha or more, 2200 g ai/ha or more, or 2220 g ai/ha or more; in an amount of 22400 g ai/ha or less, such as 2220 g ai/ha or less, 2200 g ai/ha or less, 2150 g ai/ha or less, 2100 g ai/ha or less, 2050 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, 125 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 18 g ai/ha or less, 16 g ai/ha or less, 15 g ai/ha or less, 14 g ai/ha or less, or 12 g ai/ha or less; or in an amount ranging from any of the minimum values described above to any of the maximum values described above, such as 10-2240 g ai/ha, 12-1500 g ai/ha, 14-2100 g ai/ha, 15-900 g ai/ha, 18-1600 g ai/ha, 30-1200 g ai/ha, 50-275 g ai/ha, 70-1000 g ai/ha, 100-2220 g ai/ha, 250-1300 g ai/ha, 450-2000 g ai/ha, or 500-1900 g ai/ha.

### Other Synthetic Auxin Herbicides

In some aspects, the composition contains compounds according to Formula I, and (b) aminocyclopyrachlor, benazolin, or agriculturally acceptable salts or esters thereof.

### Aminocyclopyrachlor

In some aspects, the synthetic auxin herbicide can comprise aminocyclopyrachlor or an agriculturally acceptable salt or ester thereof. Aminocyclopyrachlor, shown below, is a pyrimidine carboxylic acid herbicide that provides control of certain broadleaf weeds and woody species, e.g., in rights-of-way, industrial sites, rangeland, permanent grass pastures and natural areas. Aminocyclopyrachlor, as well as methods of making aminocyclopyrachlor, are known in the art. Its herbicidal activity is described in The Pesticide Manual, Seventeenth Edition, 2016.

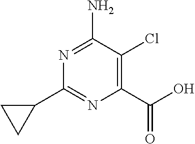 [SMILES: Nc1nc(C2CC2)nc(C(=O)O)c1Cl]

In some aspects, aminocyclopyrachlor can be provided as an agriculturally acceptable salt or ester of aminocyclopyrachlor. Exemplary agriculturally acceptable salts and esters of aminocyclopyrachlor include, but are not limited to, aminocyclopyrachlor-potassium and aminocyclopyrachlor-methyl.

Aminocyclopyrachlor can be applied to vegetation or an area adjacent the vegetation or applied to soil or water to prevent the emergence or growth of vegetation in an amount sufficient to induce a herbicidal effect. In some aspects, aminocyclopyrachlor is applied to vegetation or an area adjacent the vegetation or applied to soil or water to prevent the emergence or growth of vegetation in an amount of 20 g ai/ha or more, such as 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, or 390 g ai/ha or more; in an amount of 400 g ai/ha or less, such as 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 25 g ai/ha or less; or in an amount ranging from any of the minimum values described above to any of the maximum values described above, such as 20-400 g ai/ha, 55-150 g ai/ha, 40-310 g ai/ha, 65-190 g ai/ha, 80-260 g ai/ha, 85-300 g ai/ha, 100-275 g ai/ha, 120-330 g ai/ha, 140-390 g ai/ha, 150-250 g ai/ha, or 25-400 g ai/ha.

### Benazolin

In some aspects, the synthetic auxin herbicide can comprise benazolin or an agriculturally acceptable salt or ester thereof. Benazolin, shown below, is a benzothiazole herbicide that provides post-emergence control of certain annual broad-leaved weeds in cereals, oilseed rape, grassland, clover, alfalfa, and flax. Benazolin, as well as methods of making benazolin, are known in the art. Its herbicidal activity is described in The Pesticide Manual, Seventeenth Edition, 2016.

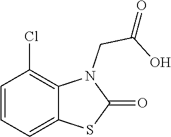 [SMILES: O=C(O)Cn1c(=O)sc2cccc(Cl)c21]

In some aspects, benazolin can be provided as an agriculturally acceptable salt or ester of benazolin. Exemplary agriculturally acceptable salts and esters of benazolin include, but are not limited to, benazolin-ethyl.

### Auxin Transport Inhibitors

In addition, the pyridine carboxylic acid herbicide or agriculturally acceptable N-oxide, salt or ester thereof, the compositions can include an auxin transport inhibitor. Auxin transport inhibitors inhibit polar transport of naturally occurring auxin, indole acetic acid (IAA), and synthetic auxin-mimicking herbicides in sensitive plants. Examples of auxin transport inhibitors include phthalamate herbicides, semicarbazone herbicides and others.

In some aspects, the composition can include an auxin transport inhibitor selected from the group consisting of: chlorflurenol; diflufenzopyr; naptalam; 2,3,5-triiodobenzoic acid (2,3,5-TIBA); agriculturally acceptable salts and esters thereof; and combinations thereof.

### Chlorflurenol

In some aspects, the auxin transport inhibitor can comprise chlorflurenol or an agriculturally acceptable salt or ester thereof. Chlorflurenol, shown below, is a herbicide that is a growth retardant and weed suppressant in amenity grass, roadsides, railways, ditch banks, etc.

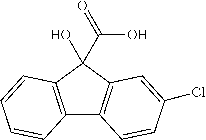 [SMILES: O=C(O)C1(O)c2ccccc2-c2ccc(Cl)cc21]

Chlorflurenol, as well as methods of preparing chlorflurenol, are known in the art. Its herbicidal activity is described, for example, in The Pesticide Manual, Seventeenth Edition, 2016.

Chlorflurenol can be applied to vegetation or an area adjacent the vegetation or applied to soil or water to prevent the emergence or growth of vegetation in an amount sufficient to induce a herbicidal effect. In some aspects, chlorflurenol is applied to vegetation or an area adjacent the vegetation or applied to soil or water to prevent the emergence or growth of vegetation in an amount of 100 g ai/ha or greater, such as 110 g ai/ha or greater, 120 g ai/ha or greater, 130 g ai/ha or greater, 140 g ai/ha or greater, 150 g ai/ha or greater, 160 g ai/ha or greater, 170 g ai/ha or greater, 180 g ai/ha or greater, 190 g ai/ha or greater, 200 g ai/ha or greater, 220 g ai/ha or greater, 240 g ai/ha or greater, 250 g ai/ha or greater, 260 g ai/ha or greater, 280 g ai/ha or greater, 300 g ai/ha or greater, 320 g ai/ha or greater, 340 g ai/ha or greater, 350 g ai/ha or greater, 360 g ai/ha or greater, 380 g ai/ha or greater, 400 g ai/ha or greater, 420 g ai/ha or greater, 440 g ai/ha or greater, 460 g ai/ha or greater, 480 g ai/ha or greater, 500 g ai/ha or greater, 525 g ai/ha or greater, 550 g ai/ha or greater, 575 g ai/ha or greater, 600 g ai/ha or greater, 625 g ai/ha or greater, 650 g ai/ha or greater, 675 g ai/ha or greater, 700 g ai/ha or greater, 725 g ai/ha or greater, 750 g ai/ha or greater, 775 g ai/ha or greater, 800 g ai/ha or greater, 825 g ai/ha or greater, 850 g ai/ha or greater, 875 g ai/ha or greater, 900 g ai/ha or greater, 925 g ai/ha or greater, 950 g ai/ha or greater, 975 g ai/ha or greater, 1000 g ai/ha or greater, 1100 g ai/ha or greater, 1200 g ai/ha or greater, 1300 g ai/ha or greater, 1400 g ai/ha or greater, 1500 g ai/ha or greater, 1600 g ai/ha or greater, 1700 g ai/ha or greater, 1800 g ai/ha or greater, 1900 g ai/ha or greater, 2000 g ai/ha or greater, 2200 g ai/ha or greater, 2400 g ai/ha or greater, 2600 g ai/ha or greater, 2800 g ai/ha or greater, 3000 g ai/ha or greater, 3200 g ai/ha or greater, 3400 g ai/ha or greater, 3600 g ai/ha or greater, 3800 g ai/ha or greater, 3850 g ai/ha or greater, 3900 g ai/ha or greater, or 3950 g ai/ha or greater; in an amount of 4000 g ai/ha or less, such as 3950 g ai/ha or less, 3900 g ai/ha or less, 3850 g ai/ha or less, 3800 g ai/ha or less, 3600 g ai/ha or less, 3400 g ai/ha or less, 3200 g ai/ha or less, 3000 g ai/ha or less, 2800 g ai/ha or less, 2600 g ai/ha or less, 2400 g ai/ha or less, 2200 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 350 g ai/ha or less, 340 g ai/ha or less, 320 g ai/ha or less, 300 g ai/ha or less, 280 g ai/ha or less, 260 g ai/ha or less, 250 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, or 110 g ai/ha or less; or in an amount ranging from any of the minimum values described above to any of the maximum values described above, such as from 100-4000 g ai/ha, from 130-750 g ai/ha, from 320-2000 g ai/ha, from 240-1500 g ai/ha, from 450-650 g ai/ha, from 120-600 g ai/ha, from 140-3300 g ai/ha, from 300-4500 g ai/ha, from 1400-3400 g ai/ha, from 750-2900 g ai/ha, or from 110-3950 g ai/ha.

### Diflufenzopyr

In some aspects, the auxin transport inhibitor can comprise diflufenzopyr or an agriculturally acceptable salt or ester thereof. Diflufenzopyr, shown below, is a semicarbazone herbicide that provides post-emergence control of annual and perennial broad-leaved weeds in maize, pastures, rangeland, and non-crop areas. Diflufenzopyr, as well as methods of preparing diflufenzopyr, are known in the art. Its herbicidal activity is described, for example, in The Pesticide Manual, Seventeenth Edition, 2016. Exemplary forms of diflufenzopyr include its sodium salt.

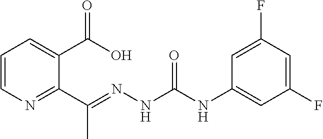 [SMILES: C/C(=N\NC(=O)Nc1cc(F)cc(F)c1)c1ncccc1C(=O)O]

Diflufenzopyr can be applied to vegetation or an area adjacent the vegetation or applied to soil or water to prevent the emergence or growth of vegetation in an amount sufficient to induce a herbicidal effect. In some aspects, diflufenzopyr is applied to vegetation or an area adjacent the vegetation or applied to soil or water to prevent the emergence or growth of vegetation in an amount of 1 g ai/ha or greater, such as 1.5 g ai/ha or greater, 2 g ai/ha or greater, 2.5 g ai/ha or greater, 3 g ai/ha or greater, 3.5 g ai/ha or greater, 4 g ai/ha or greater, 5 g ai/ha or greater, 6 g ai/ha or greater, 7 g ai/ha or greater, 8 g ai/ha or greater, 9 g ai/ha or greater, 10 g ai/ha or greater, 15 g ai/ha or greater, 20 g ai/ha or greater, 25 g ai/ha or greater, 30 g ai/ha or greater, 35 g ai/ha or greater, 40 g ai/ha or greater, 45 g ai/ha or greater, 50 g ai/ha or greater, 55 g ai/ha or greater, 60 g ai/ha or greater, 65 g ai/ha or greater, 70 g ai/ha or greater, 75 g ai/ha or greater, 80 g ai/ha or greater, 85 g ai/ha or greater, 90 g ai/ha or greater, 95 g ai/ha or greater, 100 g ai/ha or greater, 110 g ai/ha or greater, 120 g ai/ha or greater, 130 g ai/ha or greater, 140 g ai/ha or greater, 150 g ai/ha or greater, 160 g ai/ha or greater, 170 g ai/ha or greater, 180 g ai/ha or greater, 190 g ai/ha or greater, 200 g ai/ha or greater, 210 g ai/ha or greater, 220 g ai/ha or greater, 230 g ai/ha or greater, 240 g ai/ha or greater, 250 g ai/ha or greater, 260 g ai/ha or greater, 270 g ai/ha or greater, 280 g ai/ha or greater, 290 g ai/ha or greater, 300 g ai/ha or greater, 310 g ai/ha or greater, 320 g ai/ha or greater, 330 g ai/ha or greater, 340 g ai/ha or greater, 350 g ai/ha or greater, 360 g ai/ha or greater, 370 g ai/ha or greater, 380 g ai/ha or greater, 390 g ai/ha or greater, 400 g ai/ha or greater, 420 g ai/ha or greater, 440 g ai/ha or greater, 460 g ai/ha or greater, 480 g ai/ha or greater, 500 g ai/ha or greater, 520 g ai/ha or greater, 540 g ai/ha or greater, 560 g ai/ha or greater, 580 g ai/ha or greater, 600 g ai/ha or greater, 625 g ai/ha or greater, 650 g ai/ha or greater, 675 g ai/ha or greater, 700 g ai/ha or greater, 725 g ai/ha or greater, 750 g ai/ha or greater, 775 g ai/ha or greater, 800 g ai/ha or greater, 825 g ai/ha or greater, 850 g ai/ha or greater, 875 g ai/ha or greater, 900 g ai/ha or greater, 925 g ai/ha or greater, 950 g ai/ha or greater, or 975 g ai/ha or greater; in an amount of 1000 g ai/ha or less, such as 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 390 g ai/ha or less, 380 g ai/ha or less, 370 g ai/ha or less, 360 g ai/ha or less, 350 g ai/ha or less, 340 g ai/ha or less, 330 g ai/ha or less, 320 g ai/ha or less, 310 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3.5 g ai/ha or less, 3 g ai/ha or less, 2.5 g ai/ha or less, 2 g ai/ha or less, or 1.5 g ai/ha or less; or in an amount ranging from any of the minimum values described above to any of the maximum values described above, such as from 1-1000 g ai/ha, from 1-750 g ai/ha, from 3.5-750 g ai/ha, from 3.5-700 g ai/ha, from 3.5-650 g ai/ha, from 3.5-460 g ai/ha, from 3-280 g ai/ha, from 5-260 g ai/ha, from 35-240 g ai/ha, from 7-220 g ai/ha, from 2.5-500 g ai/ha, from 20-750 g ai/ha, from 1-50 g ai/ha, from 10-560 g ai/ha, from 20-500 g ai/ha, from 30-460 g ai/ha, from 40-400 g ai/ha, from 90-875 g ai/ha, from 60-300 g ai/ha, from 70-700 g ai/ha, from 100-140 g ai/ha, from 100-280 g ai/ha, or from 1.5-975 g ai/ha.

### Naptalam

In some aspects, the auxin transport inhibitor can comprise naptalam or an agriculturally acceptable salt or ester thereof. Naptalam is a phthalamate herbicide that provides pre-emergence control of many broad-leaved weeds and some grasses in cucurbits, asparagus, peanuts, soybeans, and established woody ornamentals. It is described in The Pesticide Manual, Seventeenth Edition, 2016. Exemplary forms of naptalam include its sodium salt.

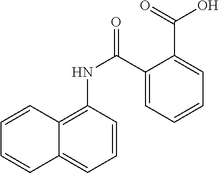 [SMILES: O=C(O)c1ccccc1C(=O)Nc1cccc2ccccc12]

Naptalam can be applied to vegetation or an area adjacent the vegetation or applied to soil or water to prevent the emergence or growth of vegetation in an amount sufficient to induce an herbicidal effect. In some aspects, naptalam is applied to vegetation or an area adjacent the vegetation or applied to soil or water to prevent the emergence or growth of vegetation in an amount of 140 g ai/ha or greater, such as 150 g ai/ha or greater, 200 g ai/ha or greater, 300 g ai/ha or greater, 400 g ai/ha or greater, 500 g ai/ha or greater, 600 g ai/ha or greater, 700 g ai/ha or greater, 750 g ai/ha or greater, 800 g ai/ha or greater, 900 g ai/ha or greater, 1000 g ai/ha or greater, 1100 g ai/ha or greater, 1200 g ai/ha or greater, 1250 g ai/ha or greater, 1300 g ai/ha or greater, 1400 g ai/ha or greater, 1500 g ai/ha or greater, 1600 g ai/ha or greater, 1700 g ai/ha or greater, 1800 g ai/ha or greater, 1900 g ai/ha or greater, 2000 g ai/ha or greater, 2200 g ai/ha or greater, 2400 g ai/ha or greater, 2600 g ai/ha or greater, 2800 g ai/ha or greater, 3000 g ai/ha or greater, 3200 g ai/ha or greater, 3400 g ai/ha or greater, 3600 g ai/ha or greater, 3800 g ai/ha or greater, 3850 g ai/ha or greater, 3900 g ai/ha or greater, 3950 g ai/ha or greater, 4000 g ai/ha or greater, 4050 g ai/ha or greater, 4100 g ai/ha or greater, 4150 g ai/ha or greater, 4200 g ai/ha or greater, 4250 g ai/ha or greater, 4300 g ai/ha or greater, 4350 g ai/ha or greater, 4400 g ai/ha or greater, 4450 g ai/ha or greater, 4500 g ai/ha or greater, 4550 g ai/ha or greater, 4600 g ai/ha or greater, 4650 g ai/ha or greater, 4700 g ai/ha or greater, 4750 g ai/ha or greater, 4800 g ai/ha or greater, 4850 g ai/ha or greater, 4900 g ai/ha or greater, 4950 g ai/ha or greater, 5000 g ai/ha or greater, 5050 g ai/ha or greater, 5100 g ai/ha or greater, 5150 g ai/ha or greater, 5200 g ai/ha or greater, 5250 g ai/ha or greater, 5300 g ai/ha or greater, 5350 g ai/ha or greater, 5400 g ai/ha or greater, or 5450 g ai/ha or greater; in an amount of 5500 g ai/ha or less, such as 5450 g ai/ha or less, 5400 g ai/ha or less, 5350 g ai/ha or less, 5300 g ai/ha or less, 5250 g ai/ha or less, 5200 g ai/ha or less, 5150 g ai/ha or less, 5100 g ai/ha or less, 5050 g ai/ha or less, 5000 g ai/ha or less, 4950 g ai/ha or less, 4900 g ai/ha or less, 4850 g ai/ha or less, 4800 g ai/ha or less, 4750 g ai/ha or less, 4700 g ai/ha or less, 4650 g ai/ha or less, 4600 g ai/ha or less, 4550 g ai/ha or less, 4500 g ai/ha or less, 4450 g ai/ha or less, 4400 g ai/ha or less, 4350 g ai/ha or less, 4300 g ai/ha or less, 4250 g ai/ha or less, 4200 g ai/ha or less, 4150 g ai/ha or less, 4100 g ai/ha or less, 4050 g ai/ha or less, 4000 g ai/ha or less, 3950 g ai/ha or less, 3900 g ai/ha or less, 3850 g ai/ha or less, 3800 g ai/ha or less, 3600 g ai/ha or less, 3400 g ai/ha or less, 3200 g ai/ha or less, 3000 g ai/ha or less, 2800 g ai/ha or less, 2600 g ai/ha or less, 2400 g ai/ha or less, 2200 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1250 g ai/ha or less, 1200 g ai/ha or less, 1100 g ai/ha or less, 1000 g ai/ha or less, 900 g ai/ha or less, 800 g ai/ha or less, 750 g ai/ha or less, 700 g ai/ha or less, 600 g ai/ha or less, 500 g ai/ha or less, 400 g ai/ha or less, 300 g ai/ha or less, 200 g ai/ha or less, 150 g ai/ha or less, or 140 g ai/ha or less; or in an amount ranging from any of the minimum values described above to any of the maximum values described above, such as from 140-5500 g ai/ha, 2400-3000 g ai/ha, 3000-5500 g ai/ha, 700-1700 g ai/ha, 300-3500 g ai/ha, 750-4000 g ai/ha, 4250-5400 g ai/ha, 2600-4400 g ai/ha, 3000-4000 g ai/ha, 1600-2800 g ai/ha, 1900-4100 g ai/ha, 1500-3600 g ai/ha, or 150-5450 g ai/ha.

2,3,5-Triiodobenzoic acid (2,3,5-TIBA)

In some aspects, the auxin transport inhibitor can comprise 2,3,5-triiodobenzoic acid (2,3,5-TIBA) or an agriculturally acceptable salt or ester thereof. 2,3,5-TIBA is an auxin transport inhibitor that inhibits plant top growth, dwarves plants, promotes the growth of lateral buds, accelerates bud formation and flowering, and promotes rooting.

2,3,5-TIBA can be applied to vegetation or an area adjacent the vegetation or applied

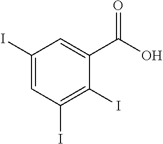 [SMILES: O=C(O)c1cc(I)cc(I)c1I]

to soil or water to prevent the emergence or growth of vegetation in an amount sufficient to induce an herbicidal effect. In some aspects, 2,3,5-TIBA is applied to vegetation or an area adjacent the vegetation or applied to soil or water to prevent the emergence or growth of vegetation in an amount of 140 g ai/ha or greater, such as 150 g ai/ha or greater, 200 g ai/ha or greater, 300 g ai/ha or greater, 400 g ai/ha or greater, 500 g ai/ha or greater, 600 g ai/ha or greater, 700 g ai/ha or greater, 750 g ai/ha or greater, 800 g ai/ha or greater, 900 g ai/ha or greater, 1000 g ai/ha or greater, 1100 g ai/ha or greater, 1200 g ai/ha or greater, 1250 g ai/ha or greater, 1300 g ai/ha or greater, 1400 g ai/ha or greater, 1500 g ai/ha or greater, 1600 g ai/ha or greater, 1700 g ai/ha or greater, 1800 g ai/ha or greater, 1900 g ai/ha or greater, 2000 g ai/ha or greater, 2200 g ai/ha or greater, 2400 g ai/ha or greater, 2600 g ai/ha or greater, 2800 g ai/ha or greater, 3000 g ai/ha or greater, 3200 g ai/ha or greater, 3400 g ai/ha or greater, 3600 g ai/ha or greater, 3800 g ai/ha or greater, 3850 g ai/ha or greater, 3900 g ai/ha or greater, 3950 g ai/ha or greater, 4000 g ai/ha or greater, 4050 g ai/ha or greater, 4100 g ai/ha or greater, 4150 g ai/ha or greater, 4200 g ai/ha or greater, 4250 g ai/ha or greater, 4300 g ai/ha or greater, 4350 g ai/ha or greater, 4400 g ai/ha or greater, 4450 g ai/ha or greater, 4500 g ai/ha or greater, 4550 g ai/ha or greater, 4600 g ai/ha or greater, 4650 g ai/ha or greater, 4700 g ai/ha or greater, 4750 g ai/ha or greater, 4800 g ai/ha or greater, 4850 g ai/ha or greater, 4900 g ai/ha or greater, 4950 g ai/ha or greater, 5000 g ai/ha or greater, 5050 g ai/ha or greater, 5100 g ai/ha or greater, 5150 g ai/ha or greater, 5200 g ai/ha or greater, 5250 g ai/ha or greater, 5300 g ai/ha or greater, 5350 g ai/ha or greater, 5400 g ai/ha or greater, or 5450 g ai/ha or greater; in an amount of 5500 g ai/ha or less, such as 5450 g ai/ha or less, 5400 g ai/ha or less, 5350 g ai/ha or less, 5300 g ai/ha or less, 5250 g ai/ha or less, 5200 g ai/ha or less, 5150 g ai/ha or less, 5100 g ai/ha or less, 5050 g ai/ha or less, 5000 g ai/ha or less, 4950 g ai/ha or less, 4900 g ai/ha or less, 4850 g ai/ha or less, 4800 g ai/ha or less, 4750 g ai/ha or less, 4700 g ai/ha or less, 4650 g ai/ha or less, 4600 g ai/ha or less, 4550 g ai/ha or less, 4500 g ai/ha or less, 4450 g ai/ha or less, 4400 g ai/ha or less, 4350 g ai/ha or less, 4300 g ai/ha or less, 4250 g ai/ha or less, 4200 g ai/ha or less, 4150 g ai/ha or less, 4100 g ai/ha or less, 4050 g ai/ha or less, 4000 g ai/ha or less, 3950 g ai/ha or less, 3900 g ai/ha or less, 3850 g ai/ha or less, 3800 g ai/ha or less, 3600 g ai/ha or less, 3400 g ai/ha or less, 3200 g ai/ha or less, 3000 g ai/ha or less, 2800 g ai/ha or less, 2600 g ai/ha or less, 2400 g ai/ha or less, 2200 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1250 g ai/ha or less, 1200 g ai/ha or less, 1100 g ai/ha or less, 1000 g ai/ha or less, 900 g ai/ha or less, 800 g ai/ha or less, 750 g ai/ha or less, 700 g ai/ha or less, 600 g ai/ha or less, 500 g ai/ha or less, 400 g ai/ha or less, 300 g ai/ha or less, 200 g ai/ha or less, 150 g ai/ha or less, or 140 g ai/ha or less; or in an amount ranging from any of the minimum values described above to any of the maximum values described above, such as from 140-5500 g ai/ha, 2400-3000 g ai/ha, 300-5500 g ai/ha, 700-2000 g ai/ha, 1250-4500 g ai/ha, 2800-5000 g ai/ha, 4250-5400 g ai/ha, 2600-4400 g ai/ha, 3000-4000 g ai/ha, 600-2800 g ai/ha, 1900-4100 g ai/ha, 1500-3600 g ai/ha, or 150-5450 g ai/ha.

### PPO Inhibitor Herbicides

In addition to the compounds according to Formula I of Formula (I), or agriculturally acceptable N-oxide, salt, or ester thereof, the compositions can include an inhibitor of a protoporphyrinogen oxidase (PPO), which is an enzyme involved in the biosynthesis of both heme and chlorophyll. It is believed that PPO inhibitors generate large amounts of singlet oxygen, which leads to the peroxidation of the lipids in cell membranes. Examples of PPO inhibitors include acifluorfen, azafenidin, benzfendizone, bifenox, butafenacil, carfentrazone, chlomethoxyfen, cinidon, fluazolate, flufenpyr, flumiclorac, flumioxazin, fluoroglycofen, fluthiacet, fomesafen, halosafen, lactofen, oxadiargyl, oxadiazon, oxyfluorfen, pentoxazone, profluazol, pyraclonil, pyraflufen, saflufenacil, sulfentrazone, thidiazimin, tiafenacil, trifludimoxazin, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxylacetate (CAS 353292-31-6; S-3100), N-ethyl-3-(2,6-dichloro-4-trifluoromethylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1-H-pyrazole-1-carboxamide (CAS 915396-43-9), N-ethyl-3-(2-chloro-6-fluoro-4-trifluoromethylphenoxy)-5-methyl-1H-pyrazo-le-1-carboxamide (CAS 452099 May 7), N-tetrahydrofurfuryl-3-(2-chloro-6-fluoro-4-trifluoromethylphenoxy)-5-methyl-1H-pyrazole-1-carboxamide (CAS 45100-03-7), 3-[7-fluoro-3-oxo-4-(prop-2-ynyl)-3,4-dihydro-2H-benzo[1,4]oxazin-6-yl]-1,5-dimethyl-6-thioxo[1,3,5]triazinan-2,4-dione, agriculturally acceptable esters thereof, or combinations thereof.

The PPO inhibitor herbicides or agriculturally acceptable salt or ester thereof can be applied to vegetation or an area adjacent to the vegetation or applied to soil or water to prevent the emergence or growth of vegetation in an amount sufficient to induce a herbicidal effect. In some aspects, the PPO inhibitor or agriculturally acceptable salt or ester thereof is applied to vegetation or an area adjacent the vegetation or applied to soil or water to prevent the emergence or growth of vegetation in an amount of 0.5 grams active ingredient per hectare (g ai/ha) or more, such as 1 g ai/ha or more, 1.25 g ai/ha or more, 1.5 g ai/ha or more, 1.75 g ai/ha or more, 2 g ai/ha or more, 2.5 g ai/ha or more, 3 g ai/ha or more, 3.5 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 17 g ai/ha or more, 18 g ai/ha or more, 19 g ai/ha or more, 20 g ai/ha or more, 21 g ai/ha or more, 22 g ai/ha or more, 23 g ai/ha or more, 24 g ai/ha or more, 25 g ai/ha or more, 26 g ai/ha or more, 27 g ai/ha or more, 28 g ai/ha or more, 29 g ai/ha or more, 30 g ai/ha or more, 31 g ai/ha or more, 32 g ai/ha or more, 33 g ai/ha or more, 34 g ai/ha or more, 35 g ai/ha or more, 36 g ai/ha or more, 37 g ai/ha or more, 38 g ai/ha or more, 39 g ai/ha or more, 40 g ai/ha or more, 41 g ai/ha or more, 42 g ai/ha or more, 43 g ai/ha or more, 44 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 250 g ai/ha or more, 260 g ai/ha or more, 280 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, or 975 g ai/ha or more; in an amount of 1000 g ai/ha or less, such as 975 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 380 g ai/ha or less, 360 g ai/ha or less, 350 g ai/ha or less, 340 g ai/ha or less, 320 g ai/ha or less, 300 g ai/ha or less, 280 g ai/ha or less, 260 g ai/ha or less, 250 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 44 g ai/ha or less, 43 g ai/ha or less, 42 g ai/ha or less, 41 g ai/ha or less, 40 g ai/ha or less, 39 g ai/ha or less, 38 g ai/ha or less, 37 g ai/ha or less, 36 g ai/ha or less, 35 g ai/ha or less, 34 g ai/ha or less, 33 g ai/ha or less, 32 g ai/ha or less, 31 g ai/ha or less, 30 g ai/ha or less, 29 g ai/ha or less, 28 g ai/ha or less, 27 g ai/ha or less, 26 g ai/ha or less, 25 g ai/ha or less, 24 g ai/ha or less, 23 g ai/ha or less, 22 g ai/ha or less, 21 g ai/ha or less, 20 g ai/ha or less, 19 g ai/ha or less, 18 g ai/ha or less, 17 g ai/ha or less, 16 ai/ha or less, 15 g ai/ha or less, 14 g ai/ha or less, 13 g ai/ha or less, 12 g ai/ha or less, 11 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3.5 g ai/ha or less, 3 g ai/ha or less, 2.5 g ai/ha or less, 2 g ai/ha or less, 1.75 g ai/ha or less, 1.5 g ai/ha or less, 1.25 g ai/ha or less, or 1 g ai/ha or less; or in an amount ranging from any of the minimum values described above to any of the maximum values described above, such as 0.5-1000 g ai/ha, 2-900 g ai/ha, 1.75-300 g ai/ha, 75-550 g ai/ha, 90-900 g ai/ha, 55-400 g ai/ha, 36-250 g ai/ha, 80-650 g ai/ha, 120-360 g ai/ha, 65-170 g ai/ha, 34-700 g ai/ha, 12-200 g ai/ha, 5-220 g ai/ha, 1.5-25 g ai/ha, 70-450 g ai/ha, 39-110 g ai/ha, or 1-975 g ai/ha.

In some aspects, the herbicidal composition contains a herbicidally effective amount of compounds according to Formula I, and (b) acifluorfen, azafenidin, benzfendizone, bifenox, butafenacil, carfentrazone, chlomethoxyfen, cinidon, fluazolate, flufenpyr, flumiclorac, flumioxazin, fluoroglycofen, fluthiacet, fomesafen, halosafen, lactofen, oxadiargyl, oxadiazon, oxyfluorfen, pentoxazone, profluazol, pyraclonil, pyraflufen, saflufenacil, sulfentrazone, thidiazimin, tiafenacil, trifludimoxazin, agriculturally acceptable salts or esters thereof, or combinations thereof.

### Acifluorfen

Compositions and methods of the present disclosure can include acifluorfen or an agriculturally acceptable salt or ester thereof. Acifluorfen, as well as methods of preparing acifluorfen, are known in the art. Acifluorfen, shown below, is 5-[2-chloro-4-(trifluoromethyl)phenoxy]-2-nitrobenzoic acid. Its herbicidal activity is exemplified in Turner, J. A., Ed., The Pesticide Manual: A World Compendium, 17^{th} ed.; BCPC: Alton, Hampshire, U K, 2016 (hereafter “The Pesticide Manual”). Exemplary uses of acifluorfen include its use for post-emergence control of broadleaf weeds, with some control of grass weeds, in soybeans, peanuts, and rice.

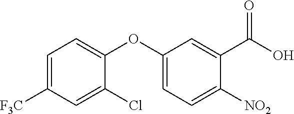 [SMILES: O=C(O)c1cc(Oc2ccc(C(F)(F)F)cc2Cl)ccc1[N+](=O)[O-]]

Acifluorfen can be applied to the foliage and roots of undesirable vegetation in an amount sufficient to induce a herbicidal effect. In some aspects, acifluorfen is applied to the foliage and roots of undesirable vegetation in an amount of 100 g ai/ha or more, such as 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 425 g ai/ha or more, 450 g ai/ha or more, 475 g ai/ha or more, 500 g ai/ha or more, 525 g ai/ha or more, 550 g ai/ha or more, 575 g ai/ha or more, 600 g ai/ha or more, 650 g ai/ha or more, or 700 g ai/ha or more; in an amount of 700 g ai/ha or less, such as 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 475 g ai/ha or less, 450 g ai/ha or less, 425 g ai/ha or less, 400 g ai/ha or less, 380 g ai/ha or less, 375 g ai/ha or less, 360 g ai/ha or less, 350 g ai/ha or less, 340 g ai/ha or less, 325 g ai/ha or less, 320 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, or 100 g ai/ha or less; or in an amount ranging from any of the minimum values described above to any of the maximum values described above, such as 100-700 g ai/ha, 120-700 g ai/ha, 225-700 g ai/ha, 200-600 g ai/ha, 100-525 g ai/ha, 160-340 g ai/ha, 200-425 g ai/ha, 140-250 g ai/ha, 200-340 g ai/ha, 110-525 g ai/ha, 125-200 g ai/ha, 100-165 g ai/ha, 120-150 g ai/ha, 125-450 g ai/ha, 100-145 g ai/ha, or 110-675 g ai/ha.

### Azafenidin

Compositions and methods of the present disclosure can include azafenidin or an agriculturally acceptable salt or ester thereof. Azafenidin, as well as methods of preparing azafenidin, are known in the art. Azafenidin, shown below, is 2-[2,4-dichloro-5-(2-propyn-1-yloxy)phenyl]-5,6,7,8-tetrahydro-1,2,4-triazolo[4,3-a]pyridin-3 (2H)-one. According to the online edition of The Pesticide Manual, azafenidin is a herbicide that was evaluated by DuPont for use as a pre-emergence and, in mixtures, post-emergence control of annual and perennial weeds in citrus, grapes, olives, sugar cane, and other perennial crops.

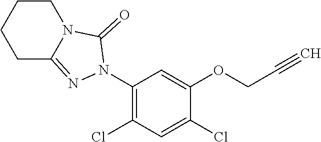 [SMILES: C#CCOc1cc(-n2nc3n(c2=O)CCCC3)c(Cl)cc1Cl]

### Benzfendizone

Compositions and methods of the present disclosure can include benzfendizone or an agriculturally acceptable salt or ester thereof. Benzfendizone, as well as methods of preparing benzfendizone, are known in the art. Benzfendizone, shown below, is methyl 2-[2-[4-[3,6-dihydro-3-methyl-2,6-dioxo-4-(trifluoromethyl)-1 (2H)-pyrimidinyl]phenoxy]methyl]-5-ethylphenoxy]propanoate. According to the online edition of The Pesticide Manual, benzfendizone is a herbicide that was evaluated by FMC for post-emergence control of grass and broadleaf weeds in orchards and no-till situations, and also as a cotton defoliant and potato desiccant.

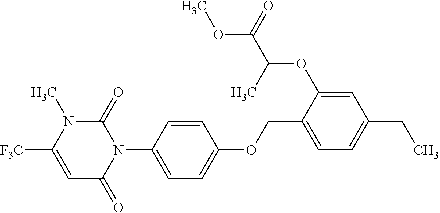 [SMILES: CCc1ccc(COc2ccc(-n3c(=O)cc(C(F)(F)F)n(C)c3=O)cc2)c(OC(C)C(=O)OC)c1]

### Bifenox

Compositions and methods of the present disclosure can include bifenox or an agriculturally acceptable salt or ester thereof. Bifenox, as well as methods of preparing bifenox, are known in the art. Bifenox, shown below, is methyl 5-(2,4-dichlorophenoxy)-2-nitrobenzoate. Its herbicidal activity is exemplified in The Pesticide Manual. Exemplary uses of bifenox include its use for pre-emergence, pre-plant incorporated, or directed post-emergence control of annual broadleaf weeds and some grass weeds in cereals, maize, sorghum, soybeans, rice, etc.

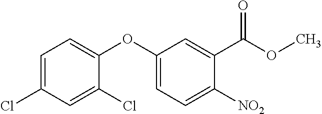 [SMILES: COC(=O)c1cc(Oc2ccc(Cl)cc2Cl)ccc1[N+](=O)[O-]]

Bifenox can be applied to the foliage, emerging shoots, and roots of undesirable vegetation in an amount sufficient to induce a herbicidal effect. In some aspects, bifenox is applied to the foliage, emerging shoots, and roots of undesirable vegetation in an amount of 50 g ai/ha or more, such as 60 g ai/ha or more, 70 g ai/ha or more, 80 g ai/ha or more, 90 g ai/ha or more, 100 g ai/ha or more, 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 350 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, or 1000 g ai/ha or more; in an amount of 1001 g ai/ha or less, such as 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, 125 g ai/ha or less, 100 g ai/ha or less, 90 g ai/ha or less, 80 g ai/ha or less, 70 g ai/ha or less, 65 g ai/ha or less, or 60 g ai/ha or less; or in an amount ranging from any of the minimum values described above to any of the maximum values described above, such as 50-1000 g ai/ha, 100-700 g ai/ha, 225-350 g ai/ha, 300-600 g ai/ha, 70-500 g ai/ha, 500-950 g ai/ha, 750-1000 g ai/ha, 60-800 g ai/ha, 75-750 g ai/ha, 150-450 g ai/ha, 200-900 g ai/ha, 325 650 g ai/ha, 90-500 g ai/ha, 175-250 g ai/ha, 140-800 g ai/ha, 450-500 g ai/ha, 125-850 g ai/ha, 65-140 g ai/ha, or 50-950 g ai/ha.

### Butafenacil

Compositions and methods of the present disclosure can include butafenacil or an agriculturally acceptable salt or ester thereof. Butafenacil, as well as methods of preparing butafenacil, are known in the art. Butafenacil, shown below, is 1,1-dimethyl-2-oxo-2-(2-propenyloxy)ethyl-2-chloro-5-[3,6-dihydro-3-methyl-2,6-dioxo-4-(trifluoromethyl)-1 (2H)-pyrimidinyl]benzoate. Its herbicidal activity is exemplified in The Pesticide Manual. Exemplary uses of butafenacil include its use for post-emergence control of annual and perennial broadleaf weeds in fruit, vineyards, citrus, and non-crop land.

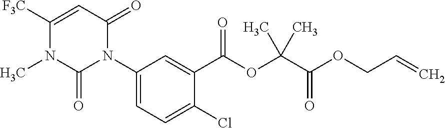 [SMILES: C=CCOC(=O)C(C)(C)OC(=O)c1cc(-n2c(=O)cc(C(F)(F)F)n(C)c2=O)ccc1Cl]

### Carfentrazone

Compositions and methods of the present disclosure can include carfentrazone or an agriculturally acceptable salt or ester thereof. Carfentrazone, as well as methods of preparing carfentrazone, are known in the art. Carfentrazone-ethyl, shown below, is a commonly used ester form of carfentrazone with the structure ethyl α,2-dichloro-5-[4-(difluoromethyl)-4,5-dihydro-3-methyl-5-oxo-1H-1,2,4-triazol-1-yl]-4-fluorobenzenepropanoate. Its herbicidal activity is exemplified in The Pesticide Manual. Exemplary uses of carfentrazone-ethyl include its use for post-emergence control of broadleaf weeds in cereals and for the desiccation of potatoes.

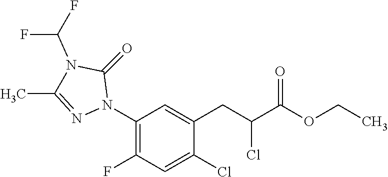 [SMILES: CCOC(=O)C(Cl)Cc1cc(-n2nc(C)n(C(F)F)c2=O)c(F)cc1Cl]

Carfentrazone can be applied to the foliage of undesirable vegetation in an amount sufficient to induce a herbicidal effect. In some aspects, carfentrazone is applied to the foliage of undesirable vegetation in an amount of 0.5 g ai/ha or more, such as 0.6 g ai/ha or more, 0.8 g ai/ha or more, 1 g ai/ha or more, 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 11 g ai/ha or more, 12.5 g ai/ha or more, 15 g ai/ha or more, 17.5 g ai/ha or more, 20 g ai/ha or more, 22.5 g ai/ha or more, 25 g ai/ha or more, 27.5 g ai/ha or more, 30 g ai/ha or more, 32.5 g ai/ha or more, 35 g ai/ha or more, 37.5 g ai/ha or more, 39 g ai/ha or more, 40 g ai/ha or more, 45 g ai/ha or more, 50 g ai/ha or more, 60 g ai/ha or more, 70 g ai/ha or more, or 75 g ai/ha or more; in an amount of 80 g ai/ha or less, such as 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 37.5 g ai/ha or less, 35 g ai/ha or less, 32.5 g ai/ha or less, 30 g ai/ha or less, 27.5 g ai/ha or less, 25 g ai/ha or less, 22.5 g ai/ha or less, 20 g ai/ha or less, 17.5 g ai/ha or less, 15 g ai/ha or less, 12.5 g ai/ha or less, 11 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, 2 g ai/ha or less, 1 g ai/ha or less, 0.8 g ai/ha or less, or 0.6 g ai/ha or less; or in an amount ranging from any of the minimum values described above to any of the maximum values described above, such as 0.5-80 g ai/ha, 1-40 g ai/ha, 3-55 g ai/ha, 5-32.5 g ai/ha, 0.8-70 g ai/ha, 15-70 g ai/ha, 7.5-40 g ai/ha, 20-30 g ai/ha, 0.6-37.5 g ai/ha, 3-30 g ai/ha, 10-25 g ai/ha, 2-17.5 g ai/ha, 40-75 g ai/ha, 4-10 g ai/ha, or 0.6-75 g ai/ha.

### Chlomethoxyfen

Compositions and methods of the present disclosure can include chlomethoxyfen or an agriculturally acceptable salt or ester thereof. Chlomethoxyfen, as well as methods of preparing chlomethoxyfen, are known in the art. Chlomethoxyfen, shown below, is 2,4-dichloro-1-(3-methoxy-4-nitrophenoxy)benzene. Its herbicidal activity is exemplified in The Pesticide Manual. Exemplary uses of chlomethoxyfen include its use for pre-emergence control of annual weeds in transplanted rice, applied at 1.5-2.5 kg ai/ha.

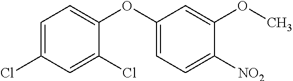 [SMILES: COc1cc(Oc2ccc(Cl)cc2Cl)ccc1[N+](=O)[O-]]

Chlomethoxyfen can be applied to the leaves and stems of undesirable vegetation in an amount sufficient to induce a herbicidal effect. In some aspects, chlomethoxyfen is applied to the leaves and stems of undesirable vegetation in an amount of 250 g ai/ha or more, such as 275 g ai/ha or more, 300 g ai/ha or more, 350 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, or 1000 g ai/ha or more; in an amount of 1001 g ai/ha or less, such as 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 375 g ai/ha or less, 350 g ai/ha or less, 325 g ai/ha or less, 300 g ai/ha or less, 275 g ai/ha or less, or 250 g ai/ha or less; or in an amount ranging from any of the minimum values described above to any of the maximum values described above, such as 250-1000 g ai/ha, 300-600 g ai/ha, 450-550 g ai/ha, 850-1000 g ai/ha, 250-750 g ai/ha, 275-900 g ai/ha, 300-400 g ai/ha, 475-500 g ai/ha, 250-475 g ai/ha, 275-350 g ai/ha, 300-325 g ai/ha, or 275-950 g ai/ha.

### Cinidon

Compositions and methods of the present disclosure can include cinidon or an agriculturally acceptable salt or ester thereof. Cinidon, as well as methods of preparing cinidon, are known in the art. Cinidon-ethyl, shown below, is a commonly used ester of cinidon with the structure ethyl (2Z)-chloro-3-[2-chloro-5-(1,3,4,5,6,7-hexahydro-1,3-dioxo-2H-isoindol-2-yl)phenyl]-2-propenoate. Its herbicidal activity is exemplified in The Pesticide Manual. Exemplary uses of cinidon include its use for post-emergence control of annual broadleaf weeds in winter and spring small grain cereals.

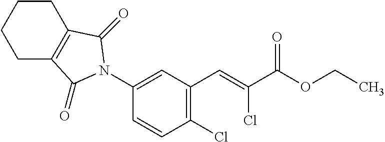 [SMILES: CCOC(=O)/C(Cl)=C/c1cc(N2C(=O)C3=C(CCCC3)C2=O)ccc1Cl]

Cinidon can be applied to the foliage of undesirable vegetation in an amount sufficient to induce a herbicidal effect. In some aspects, cinidon is applied to the foliage of undesirable vegetation in an amount of 0.5 g ai/ha or more, such as 0.6 g ai/ha or more, 0.8 g ai/ha or more, 1 g ai/ha or more, 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 22.5 g ai/ha or more, 25 g ai/ha or more, 27.5 g ai/ha or more, 30 g ai/ha or more, 32.5 g ai/ha or more, 35 g ai/ha or more, 37.5 g ai/ha or more, 39 g ai/ha or more, 40 g ai/ha or more, 45 g ai/ha or more, 50 g ai/ha or more, 60 g ai/ha or more, 70 g ai/ha or more, or 75 g ai/ha or more; in an amount of 80 g ai/ha or less, such as 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 37.5 g ai/ha or less, 35 g ai/ha or less, 32.5 g ai/ha or less, 30 g ai/ha or less, 27.5 g ai/ha or less, 25 g ai/ha or less, 22.5 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, 2 g ai/ha or less, 1 g ai/ha or less, 0.8 g ai/ha or less, or 0.6 g ai/ha or less; or in an amount ranging from any of the minimum values described above to any of the maximum values described above, such as 0.5-80 g ai/ha, 7-70 g ai/ha, 40-60 g ai/ha, 0.6-45 g ai/ha, 37.5-70 g ai/ha, 15-55 g ai/ha, 1-35 g ai/ha, 9-30 g ai/ha, 3-45 g ai/ha, 10-65 g ai/ha, 20-25 g ai/ha, 8-20 g ai/ha, 12-60 g ai/ha, 0.6-12.5 g ai/ha, or 1-75 g ai/ha.

### Fluazolate

Compositions and methods of the present disclosure can include fluazolate or an agriculturally acceptable salt or ester thereof. Fluazolate, as well as methods of preparing fluazolate, are known in the art. Fluazolate, shown below, is 1-methylethyl 5-[4-bromo-1-methyl-5-(trifluoromethyl)-1H-pyrazol-3-yl]-2-chloro-4-fluorobenzoate. According to the online edition of The Pesticide Manual, fluazolate is a herbicide under development for use as pre-emergence control of grasses and broadleaf weeds on cereals.

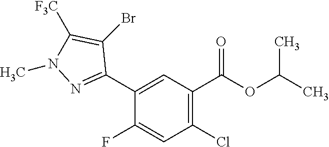 [SMILES: CC(C)OC(=O)c1cc(-c2nn(C)c(C(F)(F)F)c2Br)c(F)cc1Cl]

Fluazolate can be applied to undesirable vegetation or applied to soil to prevent the emergence or growth of undesirable vegetation in an amount sufficient to induce a herbicidal effect. In some aspects, fluazolate is applied to undesirable vegetation or applied to soil to prevent the emergence or growth of undesirable vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 90 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, or 250 g ai/ha or more; in an amount of 250 g ai/ha or less, such as 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 90 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-250 g ai/ha, 65-175 g ai/ha, 100-230 g ai/ha, 150-210 g ai/ha, 200-250 g ai/ha, 55-200 g ai/ha, 90-230 g ai/ha, 60-190 g ai/ha, 125-175 g ai/ha, 80-150 g ai/ha, 90-145 g ai/ha, 100-125 g ai/ha, 80-115 g ai/ha, or 55-240 g ai/ha.

### Flufenpyr

Compositions and methods of the present disclosure can include flufenpyr or an agriculturally acceptable salt or ester thereof. Flufenpyr, as well as methods of preparing flufenpyr, are known in the art. Flufenpyr-ethyl, shown below, is a commonly-used ester of flufenpyr, whose structure is ethyl [2-chloro-4-fluoro-5-[5-methyl-6-oxo-4-(trifluoromethyl)-1 (6H)-pyridazinyl]phenoxy]acetate. Its herbicidal activity is exemplified in The Pesticide Manual. Exemplary uses of flufenpyr include its use for control of velvetleaf and morningglory in maize, soybeans, and sugar cane.

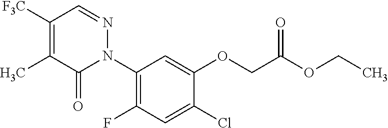 [SMILES: CCOC(=O)COc1cc(-n2ncc(C(F)(F)F)c(C)c2=O)c(F)cc1Cl]

Flufenpyr can be applied to the foliage of undesirable vegetation in an amount sufficient to induce a herbicidal effect. In some aspects, flufenpyr is applied to the foliage of undesirable vegetation in an amount of 0.5 g ai/ha or more, such as 0.6 g ai/ha or more, 0.8 g ai/ha or more, 1 g ai/ha or more, 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 22.5 g ai/ha or more, 25 g ai/ha or more, 27.5 g ai/ha or more, 30 g ai/ha or more, 32.5 g ai/ha or more, 35 g ai/ha or more, 37.5 g ai/ha or more, 39 g ai/ha or more, 40 g ai/ha or more, 45 g ai/ha or more, 50 g ai/ha or more, 60 g ai/ha or more, or 70 g ai/ha or more; in an amount of 75 g ai/ha or less, such as 70 g ai/ha or less, 65 g ai/ha or less, 60 g ai/ha or less, 55 g ai/ha or less, 50 g ai/ha or less, 45 g ai/ha or less, 40 g ai/ha or less, 37.5 g ai/ha or less, 35 g ai/ha or less, 32.5 g ai/ha or less, 30 g ai/ha or less, 27.5 g ai/ha or less, 25 g ai/ha or less, 22.5 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, 2 g ai/ha or less, 1 g ai/ha or less, 0.8 g ai/ha or less, or 0.6 g ai/ha or less; or in an amount ranging from any of the minimum values described above to any of the maximum values described above, such as 0.5-75 g ai/ha, 4-45 g ai/ha, 15-70 g ai/ha, 5-60 g ai/ha, 8-40 g ai/ha, 1-25 g ai/ha, 2-32.5 g ai/ha, 0.6-20 g ai/ha, 10-30 g ai/ha, 0.5-17.5 g ai/ha, 9-15 g ai/ha, 7-55 g ai/ha, 9-15 g ai/ha, 1-9 g ai/ha, 4-10 g ai/ha, or 0.6-70 g ai/ha.

### Flumiclorac

Compositions and methods of the present disclosure can include flumiclorac-pentyl or an agriculturally acceptable salt or ester thereof. Flumiclorac, as well as methods of preparing flumiclorac, are known in the art. Flumiclorac-pentyl, shown below, is a commonly-used ester of flumiclorac, whose structure is pentyl 2-[2-chloro-4-fluoro-5-(1,3,4,5,6,7-hexahydro-1,3-dioxo-2H-isoindol-2-yl)-phenoxy]acetate. Its herbicidal activity is exemplified in The Pesticide Manual. Exemplary uses of flumiclorac include its use for pre- and post-emergence control of broadleaf weeds in maize and soybeans, applied at 30-60 g ai/ha.

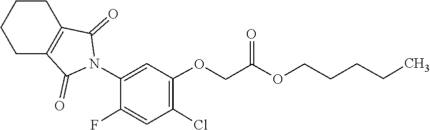 [SMILES: CCCCCOC(=O)COc1cc(N2C(=O)C3=C(CCCC3)C2=O)c(F)cc1Cl]

Flumiclorac can be applied to the foliage of undesirable vegetation in an amount sufficient to induce a herbicidal effect. In some aspects, flumiclorac is applied to the foliage of undesirable vegetation in an amount of 0.5 g ai/ha or more, such as 0.6 g ai/ha or more, 0.8 g ai/ha or more, 1 g ai/ha or more, 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 22.5 g ai/ha or more, 25 g ai/ha or more, 27.5 g ai/ha or more, 30 g ai/ha or more, 32.5 g ai/ha or more, 35 g ai/ha or more, 37.5 g ai/ha or more, 39 g ai/ha or more, 40 g ai/ha or more, 45 g ai/ha or more, 50 g ai/ha or more, 60 g ai/ha or more, 70 g ai/ha or more, or 75 g ai/ha or more; in an amount of 80 g ai/ha or less, such as 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 37.5 g ai/ha or less, 35 g ai/ha or less, 32.5 g ai/ha or less, 30 g ai/ha or less, 27.5 g ai/ha or less, 25 g ai/ha or less, 22.5 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, 2 g ai/ha or less, 1 g ai/ha or less, 0.8 g ai/ha or less, or 0.6 g ai/ha or less; or in an amount ranging from any of the minimum values described above to any of the maximum values described above, such as 0.5-80 g ai/ha, 9-75 g ai/ha, 30-60 g ai/ha, 2-50 g ai/ha, 15-70 g ai/ha, 0.5-32.5 g ai/ha, 1-25 g ai/ha, 10-65 g ai/ha, 4-20 g ai/ha, 0.8-17.5 g ai/ha, 11-35 g ai/ha, 27.5-50 g ai/ha, or 0.6-75 g ai/ha.

### Flumioxazin

Compositions and methods of the present disclosure can include flumioxazin or an agriculturally acceptable salt or ester thereof. Flumioxazin, as well as methods of preparing flumioxazin, are known in the art. Flumioxazin, shown below, is 2-[7-fluoro-3,4-dihydro-3-oxo-4-(2-propynyl)-2H-1,4-benzoxazin-6-yl]-4,5,6,7-tetrahydro-1H-isoindole-1,3 (2H)-dione. Its herbicidal activity is exemplified in The Pesticide Manual. Exemplary uses of flumioxazin include its use for pre-emergence control of annual broadleaf weeds and some annual grass weeds in soybeans, peanuts, orchards, vines, and other crops.

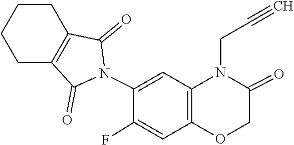 [SMILES: C#CCN1C(=O)COc2cc(F)c(N3C(=O)C4=C(CCCC4)C3=O)cc21]

Flumioxazin can be applied to the undesirable vegetation or applied to soil to prevent the emergence or growth of undesirable vegetation in an amount sufficient to induce a herbicidal effect. In some aspects, is applied to the undesirable vegetation or applied to soil to prevent the emergence or growth of undesirable vegetation in an amount of 50 g ai/ha or more, such as 60 g ai/ha or more, 70 g ai/ha or more, 75 g ai/ha or more, 80 g ai/ha or more, 90 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 220 g ai/ha or more, 240 g ai/ha or more, 250 g ai/ha or more, 260 g ai/ha or more, 280 g ai/ha or more, 300 g ai/ha or more, 325 g ai/ha or more, 350 g ai/ha or more, 375 g ai/ha or more, 400 g ai/ha or more, or 450 g ai/ha or more; in an amount of 500 g ai/ha or less, such as 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 280 g ai/ha or less, 260 g ai/ha or less, 250 g ai/ha or less, 240 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 90 g ai/ha or less, 80 g ai/ha or less, 75 g ai/ha or less, 70 g ai/ha or less, or 60 g ai/ha or less; or in an amount ranging from any of the minimum values described above to any of the maximum values described above, such as 50-500 g ai/ha, 125-300 g ai/ha, 225-500 g ai/ha, 250-500 g ai/ha, 275-475 g ai/ha, 400-450 g ai/ha, 75-200 g ai/ha, 125-260 g ai/ha, 300-375 g ai/ha, ai/ha, 60-375 g ai/ha, 80-300 g ai/ha, 90-170 g ai/ha, 125-400 g ai/ha, 100-250 g ai/ha, 50-240 g ai/ha, 90-200 g ai/ha, 110-190 g ai/ha, 75-175 g ai/ha, 60-160 g ai/ha, 90-450 g ai/ha, 100-110 g ai/ha, or 60-475 g ai/ha.

### Fluoroglycofen

Compositions and methods of the present disclosure can include fluoroglycofen or an agriculturally acceptable salt or ester thereof. Fluoroglycofen, as well as methods of preparing fluoroglycofen, are known in the art. Fluoroglycofen-ethyl, shown below, is a commonly-used ester of fluoroglycofen, whose structure is carboxymethyl 5-[2-chloro-4-(trifluoromethyl)phenoxy]-2-nitrobenzoate. Its herbicidal activity is exemplified in The Pesticide Manual. Exemplary uses of fluoroglycofen include its use for post-emergence control of broadleaf weeds and grass weeds in wheat, barley, oats, peanuts, rice, and soybeans.

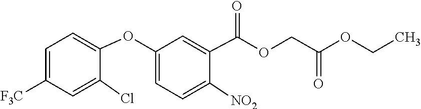 [SMILES: CCOC(=O)COC(=O)c1cc(Oc2ccc(C(F)(F)F)cc2Cl)ccc1[N+](=O)[O-]]

Fluoroglycofen thereof can be applied to the foliage or roots of undesirable vegetation in an amount sufficient to induce a herbicidal effect. In some aspects, fluoroglycofen is applied to the foliage or roots of undesirable vegetation in an amount of 0.5 g ai/ha or more, such as 0.6 g ai/ha or more, 0.8 g ai/ha or more, 1 g ai/ha or more, 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 22.5 g ai/ha or more, 25 g ai/ha or more, 27.5 g ai/ha or more, 30 g ai/ha or more, 32.5 g ai/ha or more, 35 g ai/ha or more, 37.5 g ai/ha or more, 39 g ai/ha or more, 40 g ai/ha or more, 45 g ai/ha or more, 50 g ai/ha or more, 60 g ai/ha or more, 70 g ai/ha or more, or 75 g ai/ha or more; in an amount of 80 g ai/ha or less, such as 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 37.5 g ai/ha or less, 35 g ai/ha or less, 32.5 g ai/ha or less, 30 g ai/ha or less, 27.5 g ai/ha or less, 25 g ai/ha or less, 22.5 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, 2 g ai/ha or less, 1 g ai/ha or less, 0.8 g ai/ha or less, or 0.6 g ai/ha or less; or in an amount ranging from any of the minimum values described above to any of the maximum values described above, such as 0.5-80 g ai/ha, 3-20 g ai/ha, 40-70 g ai/ha, 2-65 g ai/ha, 5-50 g ai/ha, 1-45 g ai/ha, 7-25 g ai/ha, 5-37.5 g ai/ha, 8-60 g ai/ha, 1-17.5 g ai/ha, 6-30 g ai/ha, 2-9 g ai/ha, 0.6-75 g ai/ha.

### Fluthiacet

Compositions and methods of the present disclosure can include fluthiacet or an agriculturally acceptable salt or ester thereof. Fluthiacet, as well as methods of preparing fluthiacet, are known in the art. Fluthiacet-methyl, shown below, is a commonly-used ester of fluthiacet, whose structure is methyl 2-[[2-chloro-4-fluoro-5-[(tetrahydro-3-oxo-1H,3H-[1,3,4]thiadiazolo[3,4-a]pyridazin-1-ylidene)amino]-phenyl]thio]acetate. Its herbicidal activity is exemplified in The Pesticide Manual. Exemplary uses of fluthiacet-methyl include its use for post-emergence control of broadleaf weeds in maize and soybeans, applied at 4-15 g ai/ha.

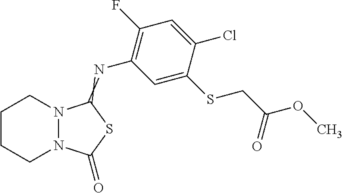 [SMILES: COC(=O)CSc1cc(N=c2sc(=O)n3n2CCCC3)c(F)cc1Cl]

Fluthiacet can be applied to the foliage of undesirable vegetation in an amount sufficient to induce a herbicidal effect. In some aspects, fluthiacet is applied to the foliage of undesirable vegetation in an amount of 0.5 g ai/ha or more, such as 0.6 g ai/ha or more, 0.8 g ai/ha or more, 1 g ai/ha or more, 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 22.5 g ai/ha or more, 25 g ai/ha or more, 27.5 g ai/ha or more, 30 g ai/ha or more, 32.5 g ai/ha or more, 35 g ai/ha or more, 37.5 g ai/ha or more, 39 g ai/ha or more, 40 g ai/ha or more, or 45 g ai/ha or more; in an amount of 50 g ai/ha or less, such as 45 g ai/ha or less, 40 g ai/ha or less, 37.5 g ai/ha or less, 35 g ai/ha or less, 32.5 g ai/ha or less, 30 g ai/ha or less, 27.5 g ai/ha or less, 25 g ai/ha or less, 22.5 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, 2 g ai/ha or less, 1 g ai/ha or less, 0.8 g ai/ha or less, or 0.6 g ai/ha or less; or in an amount ranging from any of the minimum values described above to any of the maximum values described above, such as 0.5-50 g ai/ha, 0.8-20 g, 9-45 g ai/ha, 5-37.5 g ai/ha, 10-15 g ai/ha, 20-30 g ai/ha, 7-25 g ai/ha, 1-20 g ai/ha, 3-30 g ai/ha, 12.5-45 g ai/ha, 1-10 g ai/ha, 6-40 g ai/ha, or 0.6-45 g ai/ha.

### Fomesafen

Compositions and methods of the present disclosure can include fomesafen or an agriculturally acceptable salt or ester thereof. Fomesafen, as well as methods of preparing fomesafen, are known in the art. Fomesafen, shown below, is 5-[2-chloro-4-(trifluoromethyl)phenoxy]-N-(methylsulfonyl)-2-nitrobenzamide. Its herbicidal activity is exemplified in The Pesticide Manual. Exemplary uses of fomesafen include its use for early post-emergence control of broadleaf weeds in soybeans, applied at 200-400 g ai/ha.

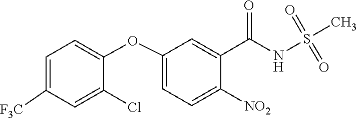 [SMILES: CS(=O)(=O)NC(=O)c1cc(Oc2ccc(C(F)(F)F)cc2Cl)ccc1[N+](=O)[O-]]

Fomesafen can be applied to the foliage and roots of undesirable vegetation in an amount sufficient to induce a herbicidal effect. In some aspects, fomesafen is applied to the foliage and roots of undesirable vegetation in an amount of 100 g ai/ha or more, such as 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 425 g ai/ha or more, 450 g ai/ha or more, or 475 g ai/ha or more; in an amount of 500 g ai/ha or less, such as 475 g ai/ha or less, 450 g ai/ha or less, 425 g ai/ha or less, 400 g ai/ha or less, 380 g ai/ha or less, 375 g ai/ha or less, 360 g ai/ha or less, 350 g ai/ha or less, 340 g ai/ha or less, 325 g ai/ha or less, 320 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, or 110 g ai/ha or less; or in an amount ranging from any of the minimum values described above to any of the maximum values described above, such as 100-500 g ai/ha, 175-300 g ai/ha, 225-475 g ai/ha, 100-400 g ai/ha, 170-375 g ai/ha, 120-250 g ai/ha, 180-300 g ai/ha, 110-250 g ai/ha, 130-210 g ai/ha, 160-200 g ai/ha, 100-180 g ai/ha, 210-375 g ai/ha, 150-275 g ai/ha, 190-450 g ai/ha, 120-140 g ai/ha, or 110-475 g ai/ha.

### Halosafen

Compositions and methods of the present disclosure can include halosafen or an agriculturally acceptable salt or ester thereof. Halosafen, as well as methods of preparing halosafen, are known in the art. Halosafen, shown below, is 5-[2-chloro-6-fluoro-4-(trifluoromethyl)phenoxy]-N-(ethylsulfonyl)-2-nitrobenzamide. According to the online edition of The Pesticide Manual, halosafen is a herbicide that was evaluated by ICI Agrochemicals.

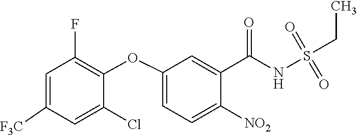 [SMILES: CCS(=O)(=O)NC(=O)c1cc(Oc2c(F)cc(C(F)(F)F)cc2Cl)ccc1[N+](=O)[O-]]

### Lactofen

Compositions and methods of the present disclosure can include lactofen or an agriculturally acceptable salt or ester thereof. Lactofen, as well as methods of preparing lactofen, are known in the art. Lactofen, shown below, is 2-ethoxy-1-methyl-2-oxoethyl 5-[2-chloro-4-(trifluoromethyl)phenoxy]-2-nitrobenzoate. Its herbicidal activity is exemplified in The Pesticide Manual. Exemplary uses of lactofen include its use for post-emergence control of broadleaf weeds cotton, soybeans, and snap beans, applied at 220 g ai/ha.

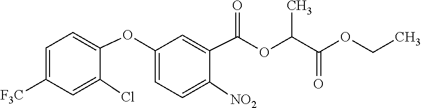 [SMILES: CCOC(=O)C(C)OC(=O)c1cc(Oc2ccc(C(F)(F)F)cc2Cl)ccc1[N+](=O)[O-]]

Lactofen can be applied to the foliage of undesirable vegetation in an amount sufficient to induce an herbicidal effect. In some aspects, lactofen is applied to the foliage of undesirable vegetation in an amount of 50 g ai/ha or more, such as 60 g ai/ha or more, 70 g ai/ha or more, 75 g ai/ha or more, 80 g ai/ha or more, 90 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 220 g ai/ha or more, 240 g ai/ha or more, 250 g ai/ha or more, 260 g ai/ha or more, 280 g ai/ha or more, 300 g ai/ha or more, 325 g ai/ha or more, 350 g ai/ha or more, 375 g ai/ha or more, 400 g ai/ha or more, or 450 g ai/ha or more; in an amount of 500 g ai/ha or less, such as 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 280 g ai/ha or less, 260 g ai/ha or less, 250 g ai/ha or less, 240 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 90 g ai/ha or less, 80 g ai/ha or less, 75 g ai/ha or less, 70 g ai/ha or less, or 60 g ai/ha or less; or in an amount ranging from any of the minimum values described above to any of the maximum values described above, such as 50-500 g ai/ha, 175-350 g ai/ha, 220-450 g ai/ha, 75-400 g ai/ha, 60-375 g ai/ha, 80-250 g ai/ha, 90-220 g ai/ha, 110-280 g ai/ha, 70-190 g ai/ha, 160-200 g ai/ha, 50-180 g ai/ha, 120-150 g ai/ha, 80-240 g ai/ha, 75-350 g ai/ha, 50-130 g ai/ha, or 60-475 g ai/ha.

### Oxadiargyl

Compositions and methods of the present disclosure can include oxadiargyl or an agriculturally acceptable salt or ester thereof. Oxadiargyl, as well as methods of preparing oxadiargyl, are known in the art. Oxadiargyl, shown below, is 3-[2,4-dichloro-5-(2-propyn-1-yloxy)phenyl]-5-(1,1-dimethylethyl)-1,3,4-oxadiazol-2 (3H)-one. Its herbicidal activity is exemplified in The Pesticide Manual. Exemplary uses of oxadiargyl include its use for pre- and early post-emergence control of broadleaf weeds, grass weeds, and annual sedge weeds in rice, applied at 50-150 g ai/ha, sunflowers, potatoes, vegetables, and sugar cane, applied at 300-500 g ai/ha, and fruit and citrus, applied at 500-1500 g ai/ha.

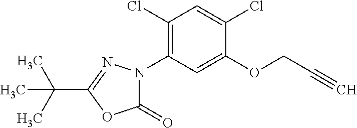 [SMILES: C#CCOc1cc(-n2nc(C(C)(C)C)oc2=O)c(Cl)cc1Cl]

Oxadiargyl thereof can be applied to undesirable vegetation or applied to soil or water to prevent the emergence or growth of undesirable vegetation in an amount sufficient to induce a herbicidal effect. In some aspects, oxadiargyl is applied to undesirable vegetation or applied to soil or water to prevent the emergence or growth of undesirable vegetation in an amount of 25 g ai/ha or more, such as 40 g ai/ha or more, 50 g ai/ha or more, 60 g ai/ha or more, 80 g ai/ha or more, 100 g ai/ha or more, 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 350 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, or 950 g ai/ha or more; in an amount of 1001 g ai/ha or less, such as 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, 125 g ai/ha or less, 100 g ai/ha or less, 80 g ai/ha or less, 60 g ai/ha or less, 50 g ai/ha or less, 40 g ai/ha or less, or 30 g ai/ha or less; or in an amount ranging from any of the minimum values described above to any of the maximum values described above, such as 25-1000 g ai/ha, 250-500 g ai/ha, 100-800 g ai/ha, 325-750 g ai/ha, 600-800 g ai/ha, 50-550 g ai/ha, 90-900 g ai/ha, 150-600 g ai/ha, 350-700 g ai/ha, 400-500 g ai/ha, 25-425 g ai/ha, 50-375 g ai/ha, 450-900 g ai/ha, 200-600 g ai/ha, 80-225 g ai/ha, 25-175 g ai/ha, 100-150 g ai/ha, 25-80 g ai/ha, or 30-950 g ai/ha.

### Oxadiazon

Compositions and methods of the present disclosure can include oxadiazon or an agriculturally acceptable salt or ester thereof. Oxadiazon, as well as methods of preparing oxadiazon, are known in the art. Oxadiazon, shown below, is 3-[2,4-dichloro-5-(1-methylethoxy)phenyl]-5-(1,1-dimethylethyl)-1,3,4-oxadiazol-2 (3H)-one. Its herbicidal activity is exemplified in The Pesticide Manual. Exemplary uses of oxadiazon include: its use for pre-emergence control of bindweed, annual broadleaf weeds, and grass weeds; post-emergence control of bindweed and annual broadleaf weeds in carnations, roses, ornamental trees and shrubs, tree fruit, bush fruit, citrus, vines, hops, cotton, rice, soybeans, sunflowers, onions, and turf; and pre-emergent or post-emergent control of annual broadleaf and grass weeds in orchards and vineyards.

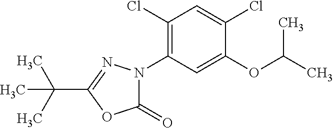 [SMILES: CC(C)Oc1cc(-n2nc(C(C)(C)C)oc2=O)c(Cl)cc1Cl]

Oxadiazon can be applied to undesirable vegetation or applied to soil or water to prevent the emergence or growth of undesirable vegetation in an amount sufficient to induce a herbicidal effect. In some aspects, oxadiazon is applied to undesirable vegetation or applied to soil or water to prevent the emergence or growth of undesirable vegetation in an amount of 250 g ai/ha or more, such as 275 g ai/ha or more, 300 g ai/ha or more, 350 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, or 950 g ai/ha or more; in an amount of 1001 g ai/ha or less, such as 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 375 g ai/ha or less, 350 g ai/ha or less, 325 g ai/ha or less, 300 g ai/ha or less, or 275 g ai/ha or less; or in an amount ranging from any of the minimum values described above to any of the maximum values described above, such as 250-1000 g ai/ha, 600-1000 g ai/ha, 350-900 g ai/ha, 275-750 g ai/ha, 400-950 g ai/ha, 650-800 g ai/ha, 250-550 g ai/ha, 325-700 g ai/ha, 450-850 g ai/ha, 250-375 g ai/ha, 300-600 g ai/ha, 250-400 g ai/ha, or 275-950 g ai/ha.

### Oxyfluorfen

Compositions and methods of the present disclosure can include oxyfluorfen or an agriculturally acceptable salt or ester thereof. Oxyfluorfen, as well as methods of preparing oxyfluorfen, are known in the art. Oxyfluorfen, shown below, is 2-chloro-1-(3-ethoxy-4-nitrophenoxy)-4-(trifluoromethyl)benzene. Its herbicidal activity is exemplified in The Pesticide Manual. Exemplary uses of oxyfluorfen include its use for pre- and post-emergence control of annual broadleaf and grass weeds in tree fruit, citrus, vines, nuts, cereals, maize, soybeans, peanuts, rice, cotton, bananas, peppermint, onions, garlic, ornamentals, and conifer seedbeds, applied at 250-2000 g ai/ha.

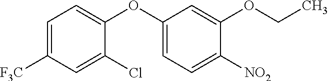 [SMILES: CCOc1cc(Oc2ccc(C(F)(F)F)cc2Cl)ccc1[N+](=O)[O-]]

Oxyfluorfen can be applied to the foliage and shoots of undesirable vegetation in an amount sufficient to induce a herbicidal effect. In some aspects, oxyfluorfen is applied to the foliage and shoots of undesirable vegetation in an amount of 50 g ai/ha or more, such as 75 g ai/ha or more, 100 g ai/ha or more, 125 g ai/ha or more, 150 g ai/ha or more, 175 g ai/ha or more, 200 g ai/ha or more, 250 g ai/ha or more, 300 g ai/ha or more, 350 g ai/ha or more, 400 g ai/ha or more, 450 g ai/ha or more, 500 g ai/ha or more, 550 g ai/ha or more, 600 g ai/ha or more, 700 g ai/ha or more, 750 g ai/ha or more, 800 g ai/ha or more, 900 g ai/ha or more, or 950 g ai/ha or more; in an amount of 1001 g ai/ha or less, such as 1000 g ai/ha or less, 900 g ai/ha or less, 800 g ai/ha or less, 750 g ai/ha or less, 700 g ai/ha or less, 600 g ai/ha or less, 550 g ai/ha or less, 500 g ai/ha or less, 450 g ai/ha or less, 400 g ai/ha or less, 350 g ai/ha or less, 300 g ai/ha or less, 250 g ai/ha or less, 200 g ai/ha or less, 175 g ai/ha or less, 150 g ai/ha or less, 125 g ai/ha or less, 100 g ai/ha or less, 75 g ai/ha or less, or 60 g ai/ha or less; or in an amount ranging from any of the minimum values described above to any of the maximum values described above, such as 50-1000 g ai/ha, 750-1000 g ai/ha, 100-900 g ai/ha, 150-750 g ai/ha, 500-800 g ai/ha, 50-600 g ai/ha, 125-500 g ai/ha, 250-900 g ai/ha, 60-450 g ai/ha, 175-300 g ai/ha, 125-250 g ai/ha, 75-200 g ai/ha, 100-175 g ai/ha, 150-800 g ai/ha, 75-550 g ai/ha, 50-125 g ai/ha, or 60-900 g ai/ha.

### Pentoxazone

Compositions and methods of the present disclosure can include pentoxazone or an agriculturally acceptable salt or ester thereof. Pentoxazone, as well as methods of preparing pentoxazone, are known in the art. Pentoxazone, shown below, is 3-(4-chloro-5-(cyclopentyloxy)-2-fluorophenyl]-5-(1-methylethylidene)-2,4-oxazolidinedione. Its herbicidal activity is exemplified in The Pesticide Manual. Exemplary uses of pentoxazone include its use for pre- and post-emergence control of Echinochloa spp. and Monochoria vaginalis in paddy rice.

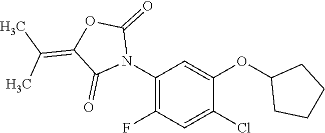 [SMILES: CC(C)=C1OC(=O)N(c2cc(OC3CCCC3)c(Cl)cc2F)C1=O]

Pentoxazone thereof can be applied to undesirable vegetation or applied to soil or water to prevent the emergence or growth of undesirable vegetation in an amount sufficient to induce a herbicidal effect. In some aspects, pentoxazone is applied to undesirable vegetation or applied to soil or water to prevent the emergence or growth of undesirable vegetation in an amount of 50 g ai/ha or more, such as 60 g ai/ha or more, 70 g ai/ha or more, 75 g ai/ha or more, 80 g ai/ha or more, 90 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 220 g ai/ha or more, 240 g ai/ha or more, 250 g ai/ha or more, 260 g ai/ha or more, 280 g ai/ha or more, 300 g ai/ha or more, 325 g ai/ha or more, 350 g ai/ha or more, 375 g ai/ha or more, 400 g ai/ha or more, 450 g ai/ha or more, or 475 g ai/ha or more; in an amount of 500 g ai/ha or less, such as 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 280 g ai/ha or less, 260 g ai/ha or less, 250 g ai/ha or less, 240 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 90 g ai/ha or less, 80 g ai/ha or less, 75 g ai/ha or less, 70 g ai/ha or less, or 60 g ai/ha or less; or in an amount ranging from any of the minimum values described above to any of the maximum values described above, such as 50-500 g ai/ha, 70-200 g ai/ha, 210-350 g ai/ha, 60-425 g ai/ha, 100-400 g ai/ha, 90-375 g ai/ha, 120-400 g ai/ha, 220-450 g ai/ha, 100-250 g ai/ha, 160-350 g ai/ha, 190-400 g ai/ha, 50-240 g ai/ha, 70-210 g ai/ha, 180-400 g ai/ha, 125-375 g ai/ha, 90-150 g ai/ha, 250-400 g ai/ha, or 60-475 g ai/ha.

### Profluazol

Compositions and methods of the present disclosure can include profluazol or an agriculturally acceptable salt or ester thereof. Profluazol, as well as methods of preparing profluazol, are known in the art. Profluazol, shown below, is 1-chloro-N-[2-chloro-4-fluoro-5-[(6S,7aR)-6-fluorotetrahydro-1,3-dioxo-1H-pyrrolo[1,2-c]imidazol-2 (3H)-yl]phenyl]methanesulfonamide. According to the online edition of The Pesticide Manual, profluazol is a herbicide developed by DuPont.

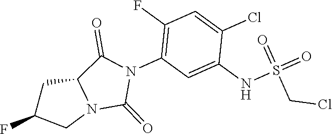 [SMILES: O=C1[C@H]2C[C@H](F)CN2C(=O)N1c1cc(NS(=O)(=O)CCl)c(Cl)cc1F]

### Pyraclonil

Compositions and methods of the present disclosure can include pyraclonil or an agriculturally acceptable salt or ester thereof. Pyraclonil, as well as methods of preparing pyraclonil, are known in the art. Pyraclonil, shown below, is 1-(3-chloro-4,5,6,7-tetrahydropyrazolo[1,5-a]pyridin-2-yl)-5-[methyl-2-propynylamino]-1H-pyrazole-4-carbonitrile. According to the online edition of The Pesticide Manual, pyraclonil is being evaluated for pre-emergence control of broadleaf and sedge weeds in rice.

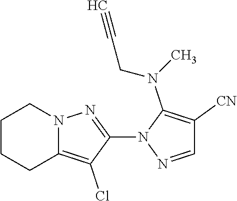 [SMILES: C#CCN(C)c1c(C#N)cnn1-c1nn2c(c1Cl)CCCC2]

Pyraclonil can be applied to undesirable vegetation or applied to soil or water to prevent the emergence or growth of undesirable vegetation in an amount sufficient to induce a herbicidal effect. In some aspects, pyraclonil is applied to undesirable vegetation or applied to soil or water to prevent the emergence or growth of undesirable vegetation in an amount of 5 g ai/ha or more, such as 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 22.5 g ai/ha or more, 25 g ai/ha or more, 27.5 g ai/ha or more, 30 g ai/ha or more, 32.5 g ai/ha or more, 35 g ai/ha or more, 37.5 g ai/ha or more, 39 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, or 95 g ai/ha or more; in an amount of 100 g ai/ha or less, such as 90 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 37.5 g ai/ha or less, 35 g ai/ha or less, 32.5 g ai/ha or less, 30 g ai/ha or less, 27.5 g ai/ha or less, 25 g ai/ha or less, 22.5 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, or 6 g ai/ha or less; or in an amount ranging from any of the minimum values described above to any of the maximum values described above, such as 5-100 g ai/ha, 40-70 g ai/ha, 8-90 g ai/ha, 6-75 g ai/ha, 15-55 g ai/ha, 50-80 g ai/ha, 25-65 g ai/ha, 15-90 g ai/ha, 9-45 g ai/ha, 5-32.5 g ai/ha, 12.5-65 g ai/ha, 20-25 g ai/ha, 6-20 g ai/ha, 10-17.5 g ai/ha, 22.5-55 g ai/ha, or 6-90 g ai/ha.

### Pyraflufen

Compositions and methods of the present disclosure can include pyraflufen or an agriculturally acceptable salt or ester thereof. Pyraflufen as well as methods of preparing pyraflufen-ethyl, are known in the art. Pyraflufen-ethyl, shown below, is a commonly used ester of pyraflufen, whose structure is ethyl 2-chloro-5-[4-chloro-5-(difluoromethoxy)-1-methyl-1H-pyrazol-3-yl]-4-fluorophenoxyacetate. Its herbicidal activity is exemplified in The Pesticide Manual. Exemplary uses of pyraflufen include its use for post-emergence control of broadleaf weeds in cereals.

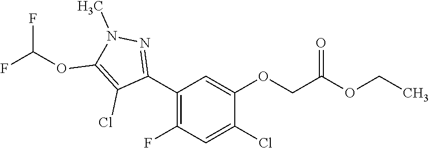 [SMILES: CCOC(=O)COc1cc(-c2nn(C)c(OC(F)F)c2Cl)c(F)cc1Cl]

Pyraflufen can be applied to the foliage of undesirable vegetation in an amount sufficient to induce a herbicidal effect. In some aspects, pyraflufen is applied to the foliage of undesirable vegetation in an amount of 0.5 g ai/ha or more, such as 0.6 g ai/ha or more, 0.8 g ai/ha or more, 1 g ai/ha or more, 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 22.5 g ai/ha or more, 25 g ai/ha or more, 27.5 g ai/ha or more, 30 g ai/ha or more, 32.5 g ai/ha or more, 35 g ai/ha or more, 37.5 g ai/ha or more, or 39 g ai/ha or more; in an amount of 40 g ai/ha or less, such as 37.5 g ai/ha or less, 35 g ai/ha or less, 32.5 g ai/ha or less, 30 g ai/ha or less, 27.5 g ai/ha or less, 25 g ai/ha or less, 22.5 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, 2 g ai/ha or less, 1 g ai/ha or less, 0.8 g ai/ha or less, or 0.6 g ai/ha or less; or in an amount ranging from any of the minimum values described above to any of the maximum values described above, such as 0.5-40 g ai/ha, 0.8-10 g, 7-40 g ai/ha, 5-37.5 g ai/ha, 1-25 g ai/ha, 15-32.5 g ai/ha, 0.6-22.5 g ai/ha, 10-20 g ai/ha, 3-15 g ai/ha, 9-30 g ai/ha, 2-10 g ai/ha, 8-35 g ai/ha, 1-5 g ai/ha, or 0.6-37.5 g ai/ha.

### Saflufenacil

Compositions and methods of the present disclosure can include saflufenacil or an agriculturally acceptable salt or ester thereof. Saflufenacil, as well as methods of preparing saflufenacil, are known in the art. Saflufenacil, shown below, is 2-chloro-5-[3,6-dihydro-3-methyl-2,6-dioxo-4-(trifluoromethyl)-1 (2H)-pyrimidinyl]-4-fluoro-N-[methyl (1-methylethyl)-amino]sulfonyl]benzamide. Its herbicidal activity is exemplified in The Pesticide Manual. Exemplary uses of saflufenacil include: pre-emergence control of broadleaf weeds in maize and sorghum; pre-plant foliar burn-down in soybeans, cereals, cotton, and legumes; and post-directed foliar burn-down in tree fruit and nuts.

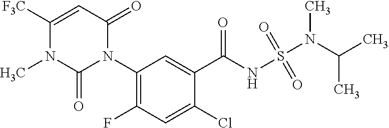 [SMILES: CC(C)N(C)S(=O)(=O)NC(=O)c1cc(-n2c(=O)cc(C(F)(F)F)n(C)c2=O)c(F)cc1Cl]

Saflufenacil can be applied to the foliage or roots of undesirable vegetation in an amount sufficient to induce a herbicidal effect. In some aspects, saflufenacil is applied to the foliage or roots of undesirable vegetation in an amount of 0.5 g ai/ha or more, such as 0.6 g ai/ha or more, 0.8 g ai/ha or more, 1 g ai/ha or more, 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 22.5 g ai/ha or more, 25 g ai/ha or more, 27.5 g ai/ha or more, 30 g ai/ha or more, 32.5 g ai/ha or more, 35 g ai/ha or more, 37.5 g ai/ha or more, 40 g ai/ha or more, 45 g ai/ha or more, 50 g ai/ha or more, 60 g ai/ha or more, 70 g ai/ha or more, or 75 g ai/ha or more; in an amount of 80 g ai/ha or less, such as 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 37.5 g ai/ha or less, 35 g ai/ha or less, 32.5 g ai/ha or less, 30 g ai/ha or less, 27.5 g ai/ha or less, 25 g ai/ha or less, 22.5 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, 2 g ai/ha or less, 1 g ai/ha or less, 0.8 g ai/ha or less, or 0.6 g ai/ha or less; or in an amount ranging from any of the minimum values described above to any of the maximum values described above, such as 0.5-80 g ai/ha, 30-75 g ai/ha, 6-70 g ai/ha, 10-65 g ai/ha, 20-50 g ai/ha, 15-80 g ai/ha, 3-45 g ai/ha, 12.5-37.5 g ai/ha, 7-35 g ai/ha, 9-32.5 g ai/ha, 1-25 g ai/ha, 8-65 g ai/ha, 4-22.5 g ai/ha, 0.5-20 g ai/ha, 0.8-17.5 g ai/ha, 22.5-55 g ai/ha, 10-40 g ai/ha, 0.5-5 g ai/ha, or 0.6-75 g ai/ha.

### Sulfentrazone

Compositions and methods of the present disclosure can include sulfentrazone or an agriculturally acceptable salt or ester thereof. Sulfentrazone, as well as methods of preparing sulfentrazone, are known in the art. Sulfentrazone, shown below, is N-[2,4-dichloro-5-[4-(difluoromethyl)-4,5-dihydro-3-methyl-5-oxo-1H-1,2,4-triazol-1-yl]phenyl]-methane-sulfonamide. Its herbicidal activity is exemplified in The Pesticide Manual. Exemplary uses of sulfentrazone include its use for pre-plant incorporated or pre-emergence control of annual broadleaf weeds, some grass weeds, and Cyperus spp. in soybeans, sugar cane, and tobacco.

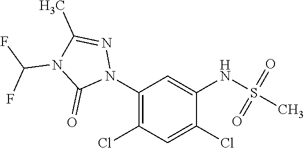 [SMILES: Cc1nn(-c2cc(NS(C)(=O)=O)c(Cl)cc2Cl)c(=O)n1C(F)F]

Sulfentrazone can be applied to the foliage and roots of undesirable vegetation in an amount sufficient to induce a herbicidal effect. In some aspects, sulfentrazone is applied to the foliage and roots of undesirable vegetation in an amount of 100 g ai/ha or more, such as 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 425 g ai/ha or more, 450 g ai/ha or more, or 475 g ai/ha or more; in an amount of 500 g ai/ha or less, such as 475 g ai/ha or less, 450 g ai/ha or less, 425 g ai/ha or less, 400 g ai/ha or less, 380 g ai/ha or less, 375 g ai/ha or less, 360 g ai/ha or less, 350 g ai/ha or less, 340 g ai/ha or less, 325 g ai/ha or less, 320 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, or 100 g ai/ha or less; or in an amount ranging from any of the minimum values described above to any of the maximum values described above, such as 100-500 g ai/ha, 250-400 g ai/ha, 120-210 g ai/ha, 375-500 g ai/ha, 200-475 g ai/ha, 360-450 g ai/ha, 120-250 g ai/ha, 110-380 g ai/ha, 125-375 g ai/ha, 170-350 g ai/ha, 220-425 g ai/ha, 150-300 g ai/ha, 120-275 g ai/ha, 160-250 g ai/ha, 280-450 g ai/ha, 130-280 g ai/ha, 110-170 g ai/ha, 120-450 g ai/ha, 100-140 g ai/ha, or 110-475 g ai/ha.

### Thidiazimin

Compositions and methods of the present disclosure can include thidiazimin or an agriculturally acceptable salt or ester thereof. Thidiazimin, as well as methods of preparing thidiazimin, are known in the art. Thidiazimin, shown below, is 6-[(Z)-(6,7-dihydro-6,6-dimethyl-3H,5H-pyrrolo[2,1-c][1,2,4]thiadiazol-3-ylidene)amino]-7-fluoro-4-(2-propyn-1-yl)-2H-1,4-benzoxazin-3 (4H)-one. Its herbicidal activity is exemplified in The Pesticide Manual. Exemplary uses of thidiazimin include its use for post-emergence control of broadleaf weeds in winter cereals, applied at 20-40 g ai/ha.

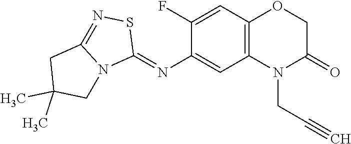 [SMILES: C#CCN1C(=O)COc2cc(F)c(/N=c3\snc4n3CC(C)(C)C4)cc21]

Thidiazimin can be applied to the foliage of undesirable vegetation in an amount sufficient to induce a herbicidal effect. In some aspects, thidiazimin thereof is applied to the foliage of undesirable vegetation in an amount of 0.5 g ai/ha or more, such as 0.6 g ai/ha or more, 0.8 g ai/ha or more, 1 g ai/ha or more, 2 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 22.5 g ai/ha or more, 25 g ai/ha or more, 27.5 g ai/ha or more, 30 g ai/ha or more, 32.5 g ai/ha or more, 35 g ai/ha or more, 37.5 g ai/ha or more, 39 g ai/ha or more, 40 g ai/ha or more, 45 g ai/ha or more, 50 g ai/ha or more, or 55 g ai/ha or more; in an amount of 60 g ai/ha or less, such as 55 g ai/ha or less, 50 g ai/ha or less, 45 g ai/ha or less, 40 g ai/ha or less, 37.5 g ai/ha or less, 35 g ai/ha or less, 32.5 g ai/ha or less, 30 g ai/ha or less, 27.5 g ai/ha or less, 25 g ai/ha or less, 22.5 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, 2 g ai/ha or less, 1 g ai/ha or less, 0.8 g ai/ha or less, or 0.6 g ai/ha or less; or in an amount ranging from any of the minimum values described above to any of the maximum values described above, such as 0.5-60 g ai/ha, 0.8-20 g ai/ha, 1-50 g ai/ha, 5-32.5 g ai/ha, 12.5-40 g ai/ha, 5-55 g ai/ha, 10-50 g ai/ha, 0.8-45 g ai/ha, 9-37.5 g ai/ha, 3-35 g ai/ha, 10-30 g ai/ha, 2-25 g ai/ha, 20-35 g ai/ha, 2-20 g ai/ha, 5-15 g ai/ha, 9-50 g ai/ha, 17.5-40 g ai/ha, or 0.6-55 g ai/ha.

### Tiafenacil

Compositions and methods of the present disclosure can include tiafenacil or an agriculturally acceptable salt or ester thereof. Tiafenacil, as well as methods of preparing tiafenacil, are known in the art. Tiafenacil, shown below, is methyl N-[2-[[2-chloro-5-[3,6-dihydro-3-methyl-2,6-dioxo-4-(trifluoromethyl)-1 (2H)-pyrimidinyl]-4-fluorophenyl]thio]-1-oxopropyl-β-alaninate. Its herbicidal activity is exemplified in The Pesticide Manual. Exemplary uses of tiafenacil include its use for none-selective post-emergence burn down control in row crops and permanent crops.

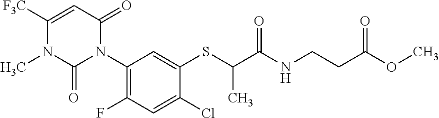

Tiafenacil can be applied to the foliage and roots of undesirable vegetation in an amount sufficient to induce a herbicidal effect. In some aspects, tiafenacil is applied to the foliage and roots of undesirable vegetation in an amount of 10 g ai/ha or more, such as 12.5 g ai/ha or more, 15 g ai/ha or more, 17.5 g ai/ha or more, 20 g ai/ha or more, 25 g ai/ha or more, 30 g ai/ha or more, 35 g ai/ha or more, 40 g ai/ha or more, 50 g ai/ha or more, 60 g ai/ha or more, or 75 g ai/ha or more, 100 g ai/ha or more, 125 g ai/ha or more, 150 g ai/ha or more, 175 g ai/ha or more, 200 g ai/ha or more, 225 g ai/ha or more, 250 g ai/ha or more, or 275 g ai/ha or more; in an amount of 300 g ai/ha or less, such as 275 g ai/ha or less, 250 g ai/ha or less, 225 g ai/ha or less, 200 g ai/ha or less, 175 g ai/ha or less, 150 g ai/ha or less, 125 g ai/ha or less, 100 g ai/ha or less, 75 g ai/ha or less, 60 g ai/ha or less, 50 g ai/ha or less, 40 g ai/ha or less, 35 g ai/ha or less, 30 g ai/ha or less, 25 g ai/ha or less, 20 g ai/ha or less, 17.5 g ai/ha or less, 15 g ai/ha or less, or 12.5 g ai/ha or less; or in an amount ranging from any of the minimum values described above to any of the maximum values described above, such as 10-300 g ai/ha, 12.5-100 g ai/ha, 40-250 g ai/ha, 275-300 g ai/ha, 10-250 g ai/ha, 50-150 g ai/ha, 175-250 g ai/ha, 17.5-225 g ai/ha, 75-200 g ai/ha, 150-250 g ai/ha, 20-175 g ai/ha, 40-150 g ai/ha, 125-150 g ai/ha, 30-200 g ai/ha, 40-125 g ai/ha, 25-100 g ai/ha, 35-275 g ai/ha, 60-150 g ai/ha, 10-75 g ai/ha, 15-70 g ai/ha, 150-225 g ai/ha, or 12.5-275 g ai/ha.

### Trifludimoxazin

Compositions and methods of the present disclosure can include mixtures of the compounds of Formula I and trifludimoxazin, a PPO inhibitor. Exemplary uses of trifludimoxazin can include pre- and/or post-emergent control of broadleaf and grass weeds. It can be applied by aircraft on citrus fruits, pome fruits, cereal grain (except rice), tree nuts, peanuts, and foliage of legume vegetables. Non-agricultural use sites include tree plantations, industrial landscaping, native grass openings, and conifer and hardwood plantations.

A composition comprising compounds according to Formula I may be mixed with or applied in combination with (b) a protoporphyrinogen oxidase (PPO) inhibitors, an agriculturally acceptable salt or ester thereof, or mixtures thereof.

In some aspects, (a) and (b) are used in an amount sufficient to induce an unexpectedly enhanced herbicidal effect (e.g., increased damage or injury to undesirable vegetation) while still showing good crop compatibility (e.g., no increased damage to crops or minimal increased damage or injury to crops) when compared to the individual application of the herbicidal compounds (a) or (b). In some aspects, the damage or injury to undesirable vegetation caused by the compositions and methods disclosed herein is evaluated using a scale from 0% to 100%, when compared with the untreated control vegetation, wherein 0% indicates no damage to the undesirable vegetation and 100% indicates complete destruction of the undesirable vegetation.

In some aspects, the joint action of (a) the compounds according to Formula I and (b) the PPO inhibitor herbicide or an agriculturally acceptable salt or ester thereof results in unexpectedly enhanced herbicidal effect against undesirable vegetation, even at application rates below those typically used for the herbicide to have a herbicidal effect on its own. In some aspects, the compositions and methods disclosed herein can, based on the individual components, be used at lower application rates to achieve a herbicidal effect comparable to the effect produced by the individual components at normal application rates.

In some aspects, the weight ratio of (a) the compounds according to Formula I (in g ae/ha) to (b) the PPO inhibitor herbicide or an agriculturally acceptable salt or ester thereof (in g ai/ha) can be 1:2000 or more, such as 1:1900 or more, 1:1500 or more, 1:1000 or more, 1:900 or more, 1:800 or more, 1:700 or more, 1:600 or more, 1:500 or more, 1:400 or more, 1:300 or more, 1:200 or more, 1:100 or more, 1:90 or more, 1:80 or more, 1:70 or more, 1:60 or more, 1:50 or more, 1:40 or more, 1:30 or more, 1:24 or more, 1:20 or more, 1:18 or more, 1:15 or more, 1:12 or more, 1:10 or more, 1:9 or more, 1:8 or more, 1:7 or more, 1:6 or more, 1:5 or more, 1:4 or more, 1:3 or more, 1:2 or more, 1:1.9 or more, 1:1.8 or more, 1:1.7 or more, 1:1.6 or more, 1:1.5 or more, 1:1.4 or more, 1:1.3 or more, 1:1.2 or more, 1:1.1 or more, 1:1 or more, 1.1:1 or more, 1.2:1 or more, 1.3:1 or more, 1.4:1 or more, 1.5:1 or more, 1.6:1 or more, 1.7:1 or more, 1.8:1 or more, 1.9:1 or more, 2:1 or more, 3:1 or more, 4:1 or more, 5:1 or more, 6:1 or more, 7:1 or more, 8:1 or more, 9:1 or more, 10:1 or more, 12:1 or more, 14:1 or more, 15:1 or more, 16:1 or more, 18:1 or more, 20:1 or more, 22:1 or more, 24:1 or more, 25:1 or more, 26:1 or more, 28:1 or more, 30:1 or more, 35:1 or more, 40:1 or more, 45:1 or more, 50:1 or more, 60:1 or more, 70:1 or more, 75:1 or more, 80:1 or more, 90:1 or more, 100:1 or more, 110:1 or more, 120:1 or more, 125:1 or more, 130:1 or more, 140:1 or more, 150:1 or more, 160:1 or more, 170:1 or more, 175:1 or more, 180:1 or more, 190:1 or more, 200:1 or more, 225:1 or more, 250:1 or more, 275:1 or more, 300:1 or more, 325:1 or more, 350:1 or more, 375:1 or more, 400:1 or more, 425:1 or more, 450:1 or more, 475:1 or more, 500:1 or more, 525:1 or more, 550:1 or more, 575:1 or more, or 590:1 or more; the weight ratio of (a) to (b) can be 600:1 or less, such as 590:1 or less, 575:1 or less, 550:1 or less, 525:1 or less, 500:1 or less, 475:1 or less, 450:1 or less, 425:1 or less, 400:1 or less, 375:1 or less, 350:1 or less, 325:1 or less, 300:1 or less, 275:1 or less, 250:1 or less, 225:1 or less, 200:1 or less, 190:1 or less, 180:1 or less, 175:1 or less, 170:1 or less, 160:1 or less, 150:1 or less, 140:1 or less, 130:1 or less, 125:1 or less, 120:1 or less, 110:1 or less, 100:1 or less, 90:1 or less, 80:1 or less, 75:1 or less, 70:1 or less, 60:1 or less, 50:1 or less, 45:1 or less, 40:1 or less, 35:1 or less, 30:1 or less, 28:1 or less, 26:1 or less, 25:1 or less, 24:1 or less, 22:1 or less, 20:1 or less, 18:1 or less, 16:1 or less, 15:1 or less, 14:1 or less, 12:1 or less, 10:1 or less, 9:1 or less, 8:1 or less, 7:1 or less, 6:1 or less, 5:1 or less, 4:1 or less, 3:1 or less, 2:1 or less, 1.9:1 or less, 1.8:1 or less, 1.7:1 or less, 1.6:1 or less, 1.5:1 or less, 1.4:1 or less, 1.3:1 or less, 1.2:1 or less, 1.1:1 or less, 1:1 or less, 1:1.1 or less, 1:1.2 or less, 1:1.3 or less, 1:1.4 or less, 1:1.5 or less, 1:1.6 or less, 1:1.7 or less, 1:1.8 or less, 1:1.9 or less, 1:2 or less, 1:3 or less, 1:4 or less, 1:5 or less, 1:6 or less, 1:7 or less, 1:8 or less, 1:9 or less, 1:10 or less, 1:20 or less, 1:30 or less, 1:40 or less, 1:50 or less, 1:60 or less, 1:70 or less, 1:80 or less, 1:90 or less, 1:100 or less, 1:200 or less, 1:300 or less, 1:400 or less, 1:500 or less, 1:1000 or less, 1:1500 or less, or 1:1900 or less; or the weight ratio of (a) to (b) can range from any of the minimum ratios to any of the maximum ratios provided above, such as from 1:2000 to 600:1, from 1:700 to 100:1, from 1:40 to 90:1, from 1:10 to 16:1, from 1:14 to 10:1, from 1:8 to 1:6, or from 1:5 to 5:1.

In some aspects, the active ingredients in the compositions disclosed herein consist of compounds according to Formula I and (b) a PPO inhibitor herbicide or an agriculturally acceptable salt or ester thereof. In some aspects, the composition may include other components, such as safeners or adjuvants, but does not include a herbicidal active ingredient in addition to (a) and (b).

In some aspects, (a) and (b), independently, can be employed in a purity of from 90% to 100% (e.g., from 95% to 100%) according to nuclear magnetic resonance (NMR) spectroscopy.

### PDS Inhibitor Herbicides

In addition to the compounds according to Formula I, the compositions can include a phytoene desaturase (PDS) inhibitor herbicide or agriculturally acceptable salt or ester thereof. PDS inhibitors block carotenoid biosynthesis by inhibition of phytoene desaturase, a key enzyme in the carotenoid biosynthesis pathway. An absence of carotenoids leads to destruction of membrane fatty acid and chlorophyll by excessive energy. Examples of PDS inhibitors include, but are not limited to, beflubutamid, diflufenican, fluridone, flurochloridone, flurtamone, norflurazon, and picolinafen.

In some aspects, the composition can include a PDS inhibitor herbicide selected from the group consisting of beflubutamid, diflufenican, fluridone, flurochloridone, flurtamone, norflurazon, picolinafen, agriculturally acceptable salts and esters thereof, and combinations thereof.

The PDS inhibitor herbicide or agriculturally acceptable salt or ester thereof can be applied to vegetation or an area adjacent the vegetation or applied to soil or water to prevent the emergence or growth of vegetation in an amount sufficient to induce a herbicidal effect. In some aspects, the PDS inhibitor herbicide or agriculturally acceptable salt or ester thereof is applied to vegetation or an area adjacent the vegetation or applied to soil or water to prevent the emergence or growth of vegetation in an amount of 12.5 gram active ingredient per hectare (g ai/ha) or more, such as 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 250 g ai/ha or more, 260 g ai/ha or more, 280 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2050 g ai/ha or more, 2100 g ai/ha or more, 2150 g ai/ha or more, 2200 g ai/ha or more, 2240 g ai/ha or more, 2250 g ai/ha or more, 2300 g ai/ha or more, 2350 g ai/ha or more, 2400 g ai/ha or more, 2450 g ai/ha or more, 2500 g ai/ha or more, 2600 g ai/ha or more, 2700 g ai/ha or more, 2750 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3900 g ai/ha or more, 3950 g ai/ha or more, 4000 g ai/ha or more, 4100 g ai/ha or more, 4200 g ai/ha or more, 4250 g ai/ha or more, 4300 g ai/ha or more, 4400 g ai/ha or more, or 4450 g ai/ha or more; in an amount of 4500 g ai/ha or less, such as 4450 g ai/ha or less, 4400 g ai/ha or less, 4300 g ai/ha or less, 4250 g ai/ha or less, 4100 g ai/ha or less, 4000 g ai/ha or less, 3950 g ai/ha or less, 3900 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2750 g ai/ha or less, 2700 g ai/ha or less, 2600 g ai/ha or less, 2500 g ai/ha or less, 2450 g ai/ha or less, 2400 g ai/ha or less, 2350 g ai/ha or less, 2300 g ai/ha or less, 2250 g ai/ha or less, 2240 g ai/ha or less, 2200 g ai/ha or less, 2150 g ai/ha or less, 2100 g ai/ha or less, 2050 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1240 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 380 g ai/ha or less, 360 g ai/ha or less, 350 g ai/ha or less, 340 g ai/ha or less, 320 g ai/ha or less, 300 g ai/ha or less, 280 g ai/ha or less, 260 g ai/ha or less, 250 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, or 15 g ai/ha or less; or in an amount ranging from any of the minimum values described above to any of the maximum values described above, such as 12.5-4500 g ai/ha, 15-2250 g ai/ha, 20-2500 g ai/ha, 25-3150 g ai/ha, 30-900 g ai/ha, 45-4200 g ai/ha, 50-1200 g ai/ha, 50-4100 g ai/ha, 60-750 g ai/ha, 75-2100 g ai/ha, 100-4000 g ai/ha, 200-3600 g ai/ha, 250-3000 g ai/ha, 250-1000 g ai/ha, 700-4250 g ai/ha, 800-1400 g ai/ha, or 1000-3700 g ai/ha.

In some aspects, the composition contains compounds according to Formula I, and (b) diflufenican, flurtamone, picolinafen, or mixtures thereof.

### Beflubutamid

In some aspects, the PDS inhibitor herbicide can comprise beflubutamid or an agriculturally acceptable salt or ester thereof. Beflubutamid, shown below, is 2-[4-fluoro-3-(trifluoromethyl)phenoxy]-N-(phenylmethyl)butanamide. Its herbicidal activity is exemplified in Tomlin, C. D. S., Ed. The Pesticide Manual: A World Compendium, 17^{th} ed.; BCPC: Alton, 2016 (hereafter “The Pesticide Manual, Seventeenth Edition, 2016”). Exemplary uses of beflubutamid include its use alone, and in mixture with isoproturon, for pre- and early post-emergence control of broadleaf weeds, such as Veronica persica, Lamium amplexicaule and Viola arvensis, in wheat and barley.

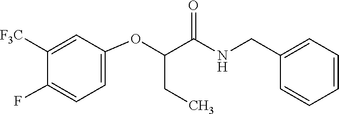 [SMILES: CCC(Oc1ccc(F)c(C(F)(F)F)c1)C(=O)NCc1ccccc1]

Beflubutamid can be applied to vegetation or an area adjacent the vegetation or applied to soil or water to prevent the emergence or growth of vegetation in an amount sufficient to induce a herbicidal effect. In some aspects, beflubutamid is applied to vegetation or an area adjacent the vegetation or applied to soil or water to prevent the emergence or growth of vegetation in an amount of 25 g ai/ha or more, such as 30 g ai/ha or more, 35 g ai/ha or more, 40 g ai/ha or more, 42.5 g ai/ha or more, 45 g ai/ha or more, 50 g ai/ha or more, 60 g ai/ha or more, 70 g ai/ha or more, 80 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 250 g ai/ha or more, 255 g ai/ha or more, 260 g ai/ha or more, 270 g ai/ha or more, 275 g ai/ha or more, 280 g ai/ha or more, or 290 g ai/ha or more; in an amount of 300 g ai/ha or less, such as 290 g ai/ha or less, 280 g ai/ha or less, 275 g ai/ha or less, 270 g ai/ha or less, 260 g ai/ha or less, 255 g ai/ha or less, 250 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 80 g ai/ha or less, 70 g ai/ha or less, 60 g ai/ha or less, 50 g ai/ha or less, 45 g ai/ha or less, 42.5 g ai/ha or less, 40 g ai/ha or less, 35 g ai/ha or less, or 30 g ai/ha or less; or in an amount ranging from any of the minimum values described above to any of the maximum values described above, such as 25-300 g ai/ha, 45-250 g ai/ha, 60-275 g ai/ha, 120-255 g ai/ha, 90-200 g ai/ha, 130-260 g ai/ha, 150-275 g ai/ha, or 42.5-300 g ai/ha.

### Diflufenican

In some aspects, the PDS inhibitor herbicide can comprise diflufenican or an agriculturally acceptable salt or ester thereof. Diflufenican, as well as methods of preparing diflufenican, are known in the art. Diflufenican, shown below, is N-(2,4-difluorophenyl)-2-[3-(trifluoromethyl)phenoxy]-3-pyridinecarboxamide. Its herbicidal activity is exemplified in The Pesticide Manual, Seventeenth Edition, 2016. Exemplary uses of diflufenican include its use as a selective contact and residual herbicide that is used pre- and early post-emergence in autumn-sown wheat and barley to control grass and broadleaf weeds.

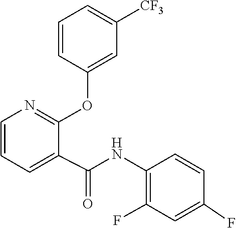 [SMILES: O=C(Nc1ccc(F)cc1F)c1cccnc1Oc1cccc(C(F)(F)F)c1]

Diflufenican can be applied to vegetation or an area adjacent the vegetation or applied to soil or water to prevent the emergence or growth of vegetation in an amount sufficient to induce a herbicidal effect. In some aspects, diflufenican is applied to vegetation or an area adjacent the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 105 g ai/ha or more, 110 g ai/ha or more, 115 g ai/ha or more, 120 g ai/ha or more, 125 g ai/ha or more, 130 g ai/ha or more, 135 g ai/ha or more, 140 g ai/ha or more, 145 g ai/ha or more, 150 g ai/ha or more, 155 g ai/ha or more, 160 g ai/ha or more, 165 g ai/ha or more, 170 g ai/ha or more, 175 g ai/ha or more, 180 g ai/ha or more, or 185 g ai/ha or more; in an amount of 187.5 g ai/ha or less, such as 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 145 g ai/ha or less, 140 g ai/ha or less, 135 g ai/ha or less, 130 g ai/ha or less, 125 g ai/ha or less, 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-187.5 g ai/ha, 55-180 g ai/ha, 60-175 g ai/ha, 65-150 g ai/ha, 70-175 g ai/ha, 75-160 g ai/ha, 80-180 g ai/ha, 80-175 g ai/ha, 85-185 g ai/ha, 85-150 g ai/ha, 90-185 g ai/ha, 90-180 g ai/ha, 90-175 g ai/ha, or 90-170 g ai/ha.

### Fluridone

In some aspects, the PDS inhibitor herbicide can comprise fluridone or an agriculturally acceptable salt or ester thereof. Fluridone, shown below, is 1-methyl-3-phenyl-5-[3-(trifluoromethyl)-phenyl]-4 (1H)-pyridinone. Its herbicidal activity is exemplified in The Pesticide Manual, Seventeenth Edition, 2016. Exemplary uses of fluridone include its use as an aquatic herbicide for control of most submerged and emerged aquatic plants (including Utricularia spp., Ceratophyllum demersum, Elodea canadensis, Myriophyllum spp., Najas guadalupensis, Potamogeton spp., Hydrilla verticillata and Panicum purpurascens) in ponds, lakes, reservoirs, irrigation ditches.

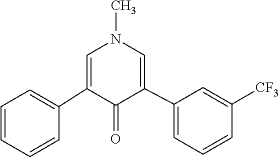 [SMILES: Cn1cc(-c2ccccc2)c(=O)c(-c2cccc(C(F)(F)F)c2)c1]

Fluridone can be applied to vegetation or an area adjacent the vegetation or applied to water to prevent the emergence or growth of vegetation in an amount sufficient to induce a herbicidal effect. In some aspects, fluridone can be applied to water to prevent the emergence or growth of vegetation in an amount of 200 g ai/ha or more, such as 220 g ai/ha or more, 240 g ai/ha or more, 250 g ai/ha or more, 260 g ai/ha or more, 280 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, or 850 g ai/ha or more; in an amount of 900 g ai/ha or less, such as 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 380 g ai/ha or less, 360 g ai/ha or less, 350 g ai/ha or less, 340 g ai/ha or less, 320 g ai/ha or less, 300 g ai/ha or less, 280 g ai/ha or less, 260 g ai/ha or less, 250 g ai/ha or less, 240 g ai/ha or less, or 220 g ai/ha or less; or in an amount ranging from any of the minimum values described above to any of the maximum values described above, such as 200-900 g ai/ha, 280-750 g ai/ha, 250-800 g ai/ha, 450-600 g ai/ha, 350-700 g ai/ha, 250-650 g ai/ha, 400-700 g ai/ha, or 220-900 g ai/ha.

### Flurochloridone

In some aspects, the PDS inhibitor herbicide can comprise flurochloridone or an agriculturally acceptable salt or ester thereof. Flurochloridone, shown below, is 3-chloro-4-(chloromethyl)-1-[3-(trifluoromethyl)phenyl]-2-pyrrolidinone. Its herbicidal activity is exemplified in The Pesticide Manual, Seventeenth Edition, 2016. Exemplary uses of flurochloridone include its use for pre-emergence control of a wide range of broadleaf and grass weeds in sunflowers, carrots and other umbelliferous crops, winter wheat, winter rye, and potatoes.

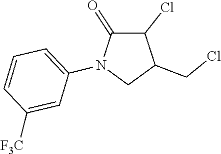 [SMILES: O=C1C(Cl)C(CCl)CN1c1cccc(C(F)(F)F)c1]

Flurochloridone can be applied to vegetation or an area adjacent the vegetation or applied to soil or water to prevent the emergence or growth of vegetation in an amount sufficient to induce a herbicidal effect. In some aspects, flurochloridone is applied to vegetation or an area adjacent the vegetation or applied to soil or water to prevent the emergence or growth of vegetation in an amount of 200 g ai/ha or more, such as 220 g ai/ha or more, 240 g ai/ha or more, 250 g ai/ha or more, 260 g ai/ha or more, 280 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, or 850 g ai/ha or more; in an amount of 900 g ai/ha or less, such as 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 380 g ai/ha or less, 360 g ai/ha or less, 350 g ai/ha or less, 340 g ai/ha or less, 320 g ai/ha or less, 300 g ai/ha or less, 280 g ai/ha or less, 260 g ai/ha or less, 250 g ai/ha or less, 240 g ai/ha or less, or 220 g ai/ha or less; or in an amount ranging from any of the minimum values described above to any of the maximum values described above, such as 200-900 g ai/ha, 220-750 g ai/ha, 250-800 g ai/ha, 450-900 g ai/ha, 350-700 g ai/ha, 250-600 g ai/ha, 400-700 g ai/ha, or 500-900 g ai/ha.

### Flurtamone

In some aspects, the PDS inhibitor herbicide can comprise flurtamone or an agriculturally acceptable salt or ester thereof. Flurtamone, shown below, is (+)-5-(methylamino)-2-phenyl-4-[3-(trifluoromethyl)phenyl]-3 (2H)-furanone. Its herbicidal activity is exemplified in The Pesticide Manual, Seventeenth Edition, 2016. Exemplary uses of flurtamone include its use for pre-plant incorporated, pre-emergence or post emergence control of broadleaf and some grass weeds in small grains, peanuts, cotton, peas and sunflowers.

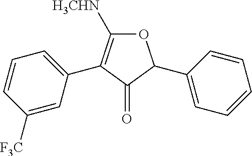

Flurtamone can be applied to vegetation or an area adjacent the vegetation or applied to soil or water to prevent the emergence or growth of vegetation in an amount sufficient to induce a herbicidal effect. In some aspects, flurtamone is applied to vegetation or an area adjacent the vegetation or applied to soil or water to prevent the emergence or growth of vegetation in an amount of 40 g ai/ha or more, such as 42.5 g ai/ha or more, 45 g ai/ha or more, 50 g ai/ha or more, 60 g ai/ha or more, 70 g ai/ha or more, 80 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 250 g ai/ha or more, 255 g ai/ha or more, 260 g ai/ha or more, 265 g ai/ha or more, 270 g ai/ha or more, 275 g ai/ha or more, 280 g ai/ha or more, 285 g ai/ha or more, 290 g ai/ha or more, 295 g ai/ha or more, 300 g ai/ha or more, 305 g ai/ha or more, 310 g ai/ha or more, 315 g ai/ha or more, 320 g ai/ha or more, 325 g ai/ha or more, 330 g ai/ha or more, 335 g ai/ha or more, 340 g ai/ha or more, 345 g ai/ha or more, 350 g ai/ha or more, 355 g ai/ha or more, 360 g ai/ha or more, 365 g ai/ha or more, or 370 g ai/ha or more; in an amount of 375 g ai/ha or less, such as 370 g ai/ha or less, 365 g ai/ha or less, 360 g ai/ha or less, 355 g ai/ha or less, 350 g ai/ha or less, 345 g ai/ha or less, 340 g ai/ha or less, 335 g ai/ha or less, 330 g ai/ha or less, 325 g ai/ha or less, 320 g ai/ha or less, 315 g ai/ha or less, 310 g ai/ha or less, 305 g ai/ha or less, 300 g ai/ha or less, 295 g ai/ha or less, 290 g ai/ha or less, 285 g ai/ha or less, 280 g ai/ha or less, 275 g ai/ha or less, 270 g ai/ha or less, 265 g ai/ha or less, 260 g ai/ha or less, or 255 g ai/ha or less, 250 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 80 g ai/ha or less, 70 g ai/ha or less, 60 g ai/ha or less, 50 g ai/ha or less, 45 g ai/ha or less, or 42.5 g ai/ha or less; or in an amount ranging from any of the minimum values described above to any of the maximum values described above, such as 40-375 g ai/ha, 125-355 g ai/ha, 60-335 g ai/ha, 80-300 g ai/ha, 265-375 g ai/ha, 170-325 g ai/ha, 45-300 g ai/ha, 190-375 g ai/ha, 120-295 g ai/ha, 180-355 g ai/ha, 220-335 g ai/ha, or 42.5-325 g ai/ha.

### Norflurazon

In some aspects, the PDS inhibitor herbicide can comprise norflurazon or an agriculturally acceptable salt or ester thereof. Norflurazon, shown below, is 4-chloro-5-(methylamino)-2-[3-(trifluoromethyl)phenyl]-3 (2H)-pyridazinone. Its herbicidal activity is exemplified in The Pesticide Manual, Seventeenth Edition, 2016. Exemplary uses of norflurazon include its use for pre-emergence control of grasses and sedges, including Digitaria spp. (crabgrasses), Echinochloa spp. (barnyard grasses), Setaria spp. (foxtails) and Eleocharis acicularis (spikerush), as well as broadleaf weeds, such as Sida spinosa (prickly sida), Portulaca oleracea (purslane), Salsola spp. (Russian thistle), and Capsella bursa-pastoris (shepherd's purse), in cotton, soybeans, peanuts, hops, orchards, ornamentals, and industrial areas.

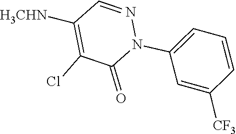

Norflurazon can be applied to vegetation or an area adjacent the vegetation or applied to soil or water to prevent the emergence or growth of vegetation in an amount sufficient to induce a herbicidal effect. In some aspects, norflurazon is applied to vegetation or an area adjacent the vegetation or applied to soil or water to prevent the emergence or growth of vegetation in an amount of 500 g ai/ha or more, such as 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2050 g ai/ha or more, 2100 g ai/ha or more, 2150 g ai/ha or more, 2200 g ai/ha or more, 2240 g ai/ha or more, 2250 g ai/ha or more, 2300 g ai/ha or more, 2350 g ai/ha or more, 2400 g ai/ha or more, 2450 g ai/ha or more, 2500 g ai/ha or more, 2600 g ai/ha or more, 2700 g ai/ha or more, 2750 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3900 g ai/ha or more, or 3950 g ai/ha or more; in an amount of 4000 g ai/ha or less, such as 3950 g ai/ha or less, 3900 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2750 g ai/ha or less, 2700 g ai/ha or less, 2600 g ai/ha or less, 2500 g ai/ha or less, 2450 g ai/ha or less, 2400 g ai/ha or less, 2350 g ai/ha or less, 2300 g ai/ha or less, 2250 g ai/ha or less, 2240 g ai/ha or less, 2200 g ai/ha or less, 2150 g ai/ha or less, 2100 g ai/ha or less, 2050 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1240 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, or 550 g ai/ha or less; or in an amount ranging from any of the minimum values described above to any of the maximum values described above, such as 500-4000 g ai/ha, 600-3600 g ai/ha, 1250-3000 g ai/ha, 2000-4000 g ai/ha, 1700-3250 g ai/ha, 2800-3950 g ai/ha, or 1000-3700 g ai/ha.

### Picolinafen

In some aspects, the PDS inhibitor herbicide can comprise picolinafen or an agriculturally acceptable salt or ester thereof. Picolinafen, shown below, is N-(4-fluorophenyl)-6-[3-(trifluoromethyl)phenoxy]-2-pyridinecarboxamide. Its herbicidal activity is exemplified in The Pesticide Manual, Seventeenth Edition, 2016. Exemplary uses of picolinafen include its use as a post-emergence herbicide either alone or in mixtures for broad-spectrum weed control in cereals.

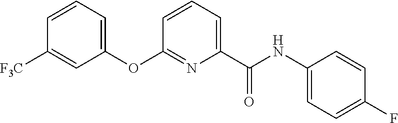 [SMILES: O=C(Nc1ccc(F)cc1)c1cccc(Oc2cccc(C(F)(F)F)c2)n1]

Picolinafen can be applied to vegetation or an area adjacent the vegetation or applied to soil or water to prevent the emergence or growth of vegetation in an amount sufficient to induce a herbicidal effect. In some aspects, picolinafen is applied to vegetation or an area adjacent the vegetation or applied to soil or water to prevent the emergence or growth of vegetation in an amount of 12.5 g ai/ha or more, such as 15 g ai/ha or more, 17.5 g ai/ha or more, 20 g ai/ha or more, 22.5 g ai/ha or more, 25 g ai/ha or more, 27.5 g ai/ha or more, 30 g ai/ha or more, 32.5 g ai/ha or more, 35 g ai/ha or more, 37.5 g ai/ha or more, 40 g ai/ha or more, 42.5 g ai/ha or more, 45 g ai/ha or more, 47.5 g ai/ha or more, 50 g ai/ha or more, 52.5 g ai/ha or more, 55 g ai/ha or more, 57.5 g ai/ha or more, 60 g ai/ha or more, 62.5 g ai/ha or more, 65 g ai/ha or more, 67.5 g ai/ha or more, 70 g ai/ha or more, 72.5 g ai/ha or more, 75 g ai/ha or more, 77.5 g ai/ha or more, 80 g ai/ha or more, 82.5 g ai/ha or more, 85 g ai/ha or more, 87.5 g ai/ha or more, 90 g ai/ha or more, 92.5 g ai/ha or more, 95 g ai/ha or more, or 97.5 g ai/ha or more; in an amount of 100 g ai/ha or less, such as 95 g ai/ha or less, 92.5 g ai/ha or less, 90 g ai/ha or less, 87.5 g ai/ha or less, 85 g ai/ha or less, 82.5 g ai/ha or less, 80 g ai/ha or less, 77.5 g ai/ha or less, 75 g ai/ha or less, 72.5 g ai/ha or less, 70 g ai/ha or less, 67.5 g ai/ha or less, 65 g ai/ha or less, 62.5 g ai/ha or less, 60 g ai/ha or less, 57.5 g ai/ha or less, 55 g ai/ha or less, 52.5 g ai/ha or less, 50 g ai/ha or less, 47.5 g ai/ha or less, 45 g ai/ha or less, 42.5 g ai/ha or less, 40 g ai/ha or less, 37.5 g ai/ha or less, 35 g ai/ha or less, 32.5 g ai/ha or less, 30 g ai/ha or less, 27.5 g ai/ha or less, 25 g ai/ha or less, 22.5 g ai/ha or less, 20 g ai/ha or less, 17.5 g ai/ha or less; or in an amount ranging from any of the minimum values described above to any of the maximum values described above, such as 12.5-100 g ai/ha, 15-90 g ai/ha, 20-80 g ai/ha, 25-75 g ai/ha, 25-95 g ai/ha, 25-80 g ai/ha, 30-75 g ai/ha, 35-90 g ai/ha, 40-95 g ai/ha, 40-75 g ai/ha, 45-95 g ai/ha, 45-90 g ai/ha, 50-80 g ai/ha, or 50-75 g ai/ha.

### Glyphosate and Glufosinate

In addition to the compounds according to Formula I, the compositions can include glyphosate, glufosinate, an agriculturally acceptable salt thereof, or mixtures thereof.

### Glyphosate

In some aspects, compositions and methods of the present disclosure can include glyphosate or an agriculturally acceptable salt thereof. Glyphosate, as well as methods of preparing glyphosate, are known in the art. Glyphosate, shown below, is N-(phosphonomethyl) glycine. Its herbicidal activity is exemplified in The Pesticide Manual, Seventeenth Edition, 2016. Exemplary uses of glyphosate include its use for control of annual and perennial grasses and broadleaf weeds, particularly in crops that have been genetically modified to be tolerant of glyphosate.

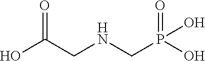

Exemplary chemical forms of glyphosate include, but are not limited to, for example, glyphosate potassium salt, glyphosate sodium salt, glyphosate monoammonium salt, glyphosate diammonium salt, glyphosate isopropylamine (IPA) salt, glyphosate monoethanolamine (MEA) salt, glyphosate monomethylamine (MMA) salt, and glyphosate dimethylamine (DMA) salt. As used herein, glyphosate salt or salt of glyphosate generally refers to the reaction product of glyphosate with a moiety that can act as a base. Typically, the reaction is an acid-base reaction.

Glyphosate can be applied to vegetation or an area adjacent the vegetation or applied to soil or water to prevent the emergence or growth of vegetation in an amount sufficient to induce a herbicidal effect. In some aspects, glyphosate is applied to vegetation or an area adjacent the vegetation or applied to soil or water to prevent the emergence or growth of vegetation in an amount of 25 g ai/ha or more, such as 30 g ai/ha or more, 50 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 105 g ai/ha or more, 110 g ai/ha or more, 115 g ai/ha or more, 120 g ai/ha or more, 125 g ai/ha or more, 130 g ai/ha or more, 135 g ai/ha or more, 140 g ai/ha or more, 145 g ai/ha or more, 150 g ai/ha or more, 155 g ai/ha or more, 160 g ai/ha or more, 165 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 500 g ai/ha or more, 600 g ai/ha or more, 700 g ai/ha or more, 750 g ai/ha or more, 800 g ai/ha or more, 900 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3800 g ai/ha or more, 3900 g ai/ha or more, 4000 g ai/ha or more, 4100 g ai/ha or more, 4200 g ai/ha or more, or 4250 g ai/ha or more, in an amount of 4300 g ai/ha or less, such as 4250 g ai/ha or less, 4200 g ai/ha or less, 4100 g ai/ha or less, 4000 g ai/ha or less, 3900 g ai/ha or less, 3800 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 900 g ai/ha or less, 800 g ai/ha or less, 750 g ai/ha or less, 700 g ai/ha or less, 600 g ai/ha or less, 500 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 145 g ai/ha or less, 140 g ai/ha or less, 135 g ai/ha or less, 130 g ai/ha or less, 125 g ai/ha or less, 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 50 g ai/ha or less, or 30 g ai/ha or less, in an amount within any range defined between any pair of the preceding values, such as 25-4300 g ai/ha, such as 50-1800 g ai/ha, 60-375 g ai/ha, 125-2500 g ai/ha, 170-1700 g ai/ha, 95-350 g ai/ha, 800-3800 g ai/ha, 185-4000 g ai/ha, 390-3700 g ai/ha, 100-420 g ai/ha, 1200-3300 g ai/ha, or 750-4250 g ai/ha.

### Glufosinate

In some aspects, compositions and methods of the present disclosure can include glufosinate or an agriculturally acceptable salt thereof. Glufosinate, shown below, is 2-amino-4-(hydroxy(methyl) phosphinyl)butanoic acid. Its herbicidal activity is exemplified in The Pesticide Manual, Seventeenth Edition, 2016. Exemplary uses of glufosinate include its use for control of annual and perennial grasses and broadleaf weeds, particularly in crops that have been genetically modified to be tolerant of glufosinate.

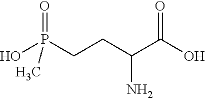

An exemplary salt of glufosinate is glufosinate-ammonium, which is also known as 2-amino-4-(hydroxy(methyl) phosphinyl)butanoic acid ammonium salt, and which has the following structure:

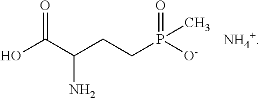

Glufosinate-ammonium is registered for controlling a wide variety of broad-leaved weeds and grasses particularly in glufosinate-tolerant crops like canola, maize, soybean, rice, cotton, and sugar beet. As used herein, glufosinate salt or salt of glufosinate generally refers to the reaction product of glufosinate with a moiety that can act as a base. Typically, the reaction is an acid-base reaction.

Other chemical forms of glufosinate include bilanafos, also known as bialaphos, which has the following structure:

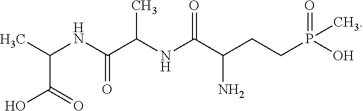 [SMILES: CC(NC(=O)C(C)NC(=O)C(N)CCP(C)(=O)O)C(=O)O]

Bilanafos also can be used in the salt form such as bilanafos sodium. Exemplary uses are described in The Pesticide Manual, Seventeenth Edition, 2016. Exemplary uses include its use to control annual and perennial broadleaf weeds and grasses.

Glufosinate can be applied to vegetation or an area adjacent the vegetation or applied to soil or water to prevent the emergence or growth of vegetation in an amount sufficient to induce a herbicidal effect. In some aspects, glufosinate is applied to vegetation or an area adjacent the vegetation or applied to soil or water to prevent the emergence or growth of vegetation in an amount of 25 g ai/ha or more, such as 50 g ai/ha or more, 70 g ai/ha or more, 75 g ai/ha or more, 90 g ai/ha or more, 100 g ai/ha or more, 112.5 g ai/ha or more, 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2050 g ai/ha or more, 2100 g ai/ha or more, 2150 g ai/ha or more, or 2200 g ai/ha or more, in an amount of 2250 g ai/ha or less (e.g., 2200 g ai/ha or less, 2150 g ai/ha or less, 2100 g ai/ha or less, 2050 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, 125 g ai/ha or less, 112.5 g ai/ha or less, 100 g ai/ha or less, 90 g ai/ha or less, 75 g ai/ha or less, 70 g ai/ha or less, or 50 g ai/ha or less, or in an amount ranging from any of the minimum values described above to any of the maximum values described above, such as in an amount of 25-2250 g ai/ha, such as 50-900 g ai/ha, 70-1800 g ai/ha, 125-475 g ai/ha, 112.5-1600 g ai/ha, 200-1300 g ai/ha, 350-2150 g ai/ha, 275-1900 g ai/ha, 400-1400 g ai/ha, 450-750 g ai/ha, 475-1100 g ai/ha, 500-2200 g ai/ha, or 650-1700 g ai/ha).

### Photosystem II Inhibitor Herbicides

In addition to the compounds according to Formula I, the compositions can include a photosystem II (PS II) inhibitor herbicide or agriculturally acceptable salt or ester thereof. Photosystem II inhibitors inhibit photosynthesis by binding to the photosystem II complex in the chloroplast. Examples of photosystem II inhibitors include phenylcarbamate herbicides, pyridazinone herbicides, triazolinone herbicides, triazine herbicides, urea herbicides, uracil herbicides, amide herbicides, nitrile herbicides, and phenylpyridazine herbicides.

In some aspects, the composition can include a PS II inhibitor herbicide selected from the group consisting of ametryne, amicarbazone, atrazine, bentazone, bromacil, bromofenoxim, bromoxynil, chlorbromuron, chloridazon, chlorotoluron, chloroxuron, cyanazine, desmedipham, desmetryn, dimefuron, dimethametryn, diuron, ethidimuron, ethiozin, fenuron, fluometuron, hexazinone, iodobonil, ioxynil, isocil, isomethiozin, isoproturon, isouron, karbutilate, lenacil, linuron, metamitron, methabenzthiazuron, metobromuron, metoxuron, metribuzin, monolinuron, neburon, pentanochlor, phenmedipham, prometon, prometryn, propanil, propazine, pyridafol, pyridate, siduron, simazine, simetryne, tebuthiuron, terbacil, terbumeton, terbuthylazine, terbutryn, trietazine, and combinations thereof.

The PS II inhibitor herbicide or agriculturally acceptable salt or ester thereof can be applied to vegetation or an area adjacent the vegetation or applied to soil or water to prevent the emergence or growth of vegetation in an amount sufficient to induce a herbicidal effect. In some aspects, the PS II inhibitor herbicide or agriculturally acceptable salt or ester thereof is applied to vegetation or an area adjacent the vegetation or applied to soil or water to prevent the emergence or growth of vegetation in an amount of 5 grams active ingredient per hectare (g ai/ha) or more, such as 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 210 g ai/ha or more, 220 g ai/ha or more, 230 g ai/ha or more, 240 g ai/ha or more, 250 g ai/ha or more, 260 g ai/ha or more, 270 g ai/ha or more, 280 g ai/ha or more, 290 g ai/ha or more, 300 g ai/ha or more, 310 g ai/ha or more, 320 g ai/ha or more, 330 g ai/ha or more, 340 g ai/ha or more, 350 g ai/ha or more, 360 g ai/ha or more, 370 g ai/ha or more, 380 g ai/ha or more, 390 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3800 g ai/ha or more, 3900 g ai/ha or more, 4000 g ai/ha or more, 5000 g ail/ha or more, 6000 g ai/ha or more, 7000 g ail/ha or more, 8000 g ai/ha or more, 9000 g ail/ha or more, 10,000 g ai/ha or more, 12,000 g ail/ha or more, 14,000 g ai/ha or more, 15,000 g ail/ha or more, 16000 g ai/ha or more, or 17,000 g ail/ha or more; in an amount of 18,000 g ai/ha or less, such as 17,000 g ai/ha or less, 16,000 g ai/ha or less, 15,000 g ai/ha or less, 14,000 g ai/ha or less, 12,000 g ai/ha or less, 10,000 g ai/ha or less, 9000 g ai/ha or less, 8000 g ai/ha or less, 7000 g ai/ha or less, 6000 g ai/ha or less, 5000 g ai/ha or less, 4000 g ai/ha or less, 3900 g ai/ha or less, 3800 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 390 g ai/ha or less, 380 g ai/ha or less, 370 g ai/ha or less, 360 g ai/ha or less, 350 g ai/ha or less, 340 g ai/ha or less, 330 g ai/ha or less, 320 g ai/ha or less, 310 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, or 10 g ai/ha or less; or in an amount ranging from any of the minimum values described above to any of the maximum values described above, such as 5-4000 g ai/ha, 5-3000 g ai/ha, 3000-4000 g ai/ha, 5-2900 g ai/ha, 5-2800 g ai/ha, 5-2700 g ai/ha, 5-2600 g ai/ha, 5-2500 g ai/ha, 5-2400 g ai/ha, 5-2300 g ai/ha, 5-2200 g ai/ha, 5-2100 g ai/ha, 5-2000 g ai/ha, 5-1800 g ai/ha, 5-1600 g ai/ha, 5-1400 g ai/ha, 5-1200 g ai/ha, 5-1000 g ai/ha, 5-900 g ai/ha, 5-800 g ai/ha, 5-700 g ai/ha, 5-600 g ai/ha, 5-500 g ai/ha, 10-4000 g ai/ha, 10-3000 g ai/ha, 10-2000 g ai/ha, 20-4000 g ai/ha, 20-3000 g ai/ha, 20-2000 g ai/ha, 20-1500 g ai/ha, 30-4000 g ai/ha, 30-3000 g ai/ha, 30-2000 g ai/ha, 50-4000 g ai/ha, 50-3000 g ai/ha, 50-2000 g ai/ha, 50-1500 g ai/ha, 70-4000 g ai/ha, 70-3000 g ai/ha, 70-2500 g ai/ha, 70-2000 g ai/ha, 70-1500 g ai/ha, 100-3000 g ai/ha, 100-2500 g ai/ha, or 100-2000 g ai/ha. In some aspects, the photosystem II inhibitor or agriculturally acceptable salt or ester thereof is applied to vegetation or an area adjacent the vegetation or applied to soil or water to prevent the emergence or growth of vegetation in an amount of from 30-1000 g ai/ha.

In some aspects, the composition contains compounds according to Formula I, and (b) ametryne, amicarbazone, atrazine, bentazone, bromacil, bromofenoxim, bromoxynil, chlorbromuron, chloridazon, chlorotoluron, chloroxuron, cyanazine, desmedipham, desmetryn, dimefuron, dimethametryn, diuron, ethidimuron, ethiozin, fenuron, fluometuron, hexazinone, iodobonil, ioxynil, isocil, isomethiozin, isoproturon, isouron, karbutilate, lenacil, linuron, metamitron, methabenzthiazuron, methoprotryne, metobromuron, metoxuron, metribuzin, monolinuron, neburon, pentanochlor, phenmedipham, prometon, prometryn, propanil, propazine, pyridafol, pyridate, siduron, simazine, simetryne, tebuthiuron, terbacil, terbumeton, terbuthylazine, terbutryn, trietazine, or mixtures thereof.

### Ametryn

In some aspects, the PS II inhibitor herbicide can comprise ametryn or an agriculturally acceptable salt or ester thereof. Ametryn, shown below, is N²-ethyl-N⁴-isopropyl-6-(methylthio)-1,3,4-triazine-2,4-diamine. Its herbicidal activity is exemplified in Tomlin, C. D. S., Ed. The Pesticide Manual: A World Compendium, 17^{th} ed.; BCPC: Alton, 2016 (hereafter “The Pesticide Manual, Seventeenth Edition, 2016”). Exemplary uses of ametryn include its use for pre- and early post-emergence control of annual grass and broadleaf weeds in pineapples, sugar cane, maize, bananas, cassava, sisal, and orchards.

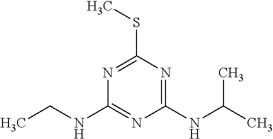

Ametryn can be applied to vegetation or an area adjacent the vegetation or applied to soil or water to prevent the emergence or growth of vegetation in an amount sufficient to induce a herbicidal effect. In some aspects, ametryn is applied to vegetation or an area adjacent the vegetation or applied to soil or water to prevent the emergence or growth of vegetation in an amount of 2000 g ai/ha or more, such as 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3800 g ai/ha or more, or 3900 g ai/ha or more; in an amount of 4000 g ai/ha or less, such as 3900 g ai/ha or less, 3800 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, or 2100 g ai/ha or less; or in an amount ranging from any of the minimum values described above to any of the maximum values described above, such as 2000-4000 g ai/ha, 2100-2500 g ai/ha, 2600-3700 g ai/ha, 2500-4000 g ai/ha, 2900-3600 g ai/ha, 3200-3800 g ai/ha, 2300-3700 g ai/ha, or 2200-3900 g ai/ha.

### Amicarbazone

In some aspects, the PDS inhibitor herbicide can comprise amicarbazone or an agriculturally acceptable salt or ester thereof. Amicarbazone, shown below, is 4-amino-N-tert-butyl-4,5-dihydro-3-isopropyl-5-oxo-1H-1,2,4-triazole-1-carboxamide. Its herbicidal activity is exemplified in The Pesticide Manual, Seventeenth Edition, 2016. Exemplary uses of amicarbazone include its use for pre-emergence control of annual broadleaf weeds in maize, pre- and post-emergence control of annual broadleaf and grass weeds in sugar cane, and post-emergence control of annual broadleaf and grass weeds in turf, and conifers.

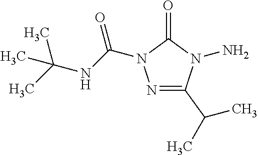 [SMILES: CC(C)c1nn(C(=O)NC(C)(C)C)c(=O)n1N]

Amicarbazone can be applied to vegetation or an area adjacent the vegetation or applied to soil or water to prevent the emergence or growth of vegetation in an amount sufficient to induce a herbicidal effect. In some aspects, amicarbazone is applied to vegetation or an area adjacent the vegetation or applied to soil or water to prevent the emergence or growth of vegetation in an amount of 100 g ai/ha or more, such as 105 g ai/ha or more, 110 g ai/ha or more, 115 g ai/ha or more, 120 g ai/ha or more, 125 g ai/ha or more, 130 g ai/ha or more, 135 g ai/ha or more, 140 g ai/ha or more, 145 g ai/ha or more, 150 g ai/ha or more, 155 g ai/ha or more, 160 g ai/ha or more, 165 g ai/ha or more, 170 g ai/ha or more, 175 g ai/ha or more, 180 g ai/ha or more, 185 g ai/ha or more, 190 g ai/ha or more, 200 g ai/ha or more, 210 g ai/ha or more, 220 g ai/ha or more, 230 g ai/ha or more, 240 g ai/ha or more, 250 g ai/ha or more, 260 g ai/ha or more, 270 g ai/ha or more, 280 g ai/ha or more, 290 g ai/ha or more, 300 g ai/ha or more, 310 g ai/ha or more, 320 g ai/ha or more, 330 g ai/ha or more, 340 g ai/ha or more, 350 g ai/ha or more, 360 g ai/ha or more, 370 g ai/ha or more, 380 g ai/ha or more, 390 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, or 975 g ai/ha or more; in an amount of 1000 g ai/ha or less, such as 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 390 g ai/ha or less, 380 g ai/ha or less, 370 g ai/ha or less, 360 g ai/ha or less, 350 g ai/ha or less, 340 g ai/ha or less, 330 g ai/ha or less, 320 g ai/ha or less, 310 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 145 g ai/ha or less, 140 g ai/ha or less, 135 g ai/ha or less, 130 g ai/ha or less, 125 g ai/ha or less, 120 g ai/ha or less, 115 g ai/ha or less, 110 g ai/ha or less, or 105 g ai/ha or less; or in an amount ranging from any of the minimum values described above to any of the maximum values described above, such as 100-1000 g ai/ha, 125-750 g ai/ha, 160-675 g ai/ha, 220-850 g ai/ha, 370-900 g ai/ha, 275-650 g ai/ha, 480-800 g ai/ha, 340-900 g ai/ha, 520-925 g ai/ha, 290-560 g ai/ha, 390-825 g ai/ha, or 105-975 g ai/ha.

### Atrazine

In some aspects, the PDS inhibitor herbicide can comprise atrazine or an agriculturally acceptable salt or ester thereof. Atrazine, shown below, is 6-chloro-N²-ethyl-N⁴-isopropyl-1,3,5-triazine-2,4-diamine. Its herbicidal activity is exemplified in The Pesticide Manual, Seventeenth Edition, 2016. Exemplary uses of atrazine include its use as a pre- and post-emergence control of annual broadleaf and grass weeds in maize, sorghum, sugar cane, pineapples, chemical fallow, grassland, macadamia nuts, conifers, and industrial weed control.

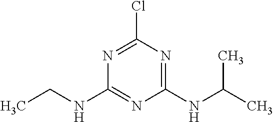

Atrazine can be applied to vegetation or an area adjacent the vegetation or applied to water to prevent the emergence or growth of vegetation in an amount sufficient to induce a herbicidal effect. In some aspects, atrazine can be applied to water to prevent the emergence or growth of vegetation in an amount of 300 g ai/ha or more, such as 320 g ai/ha or more, 340 g ai/ha or more, 360 g ai/ha or more, 380 g ai/ha or more, 420 g ai/ha or more, 440 g ai/ha or more, 460 g ai/ha or more, 480 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3800 g ai/ha or more, or 3900 g ai/ha or more; in an amount of 4000 g ai/ha or less, such as 3900 g ai/ha or less, 3800 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, or 320 g ai/ha or less; or in an amount ranging from any of the minimum values described above to any of the maximum values described above, such as 300-4000 g ai/ha, 480-2700 g ai/ha, 850-3800 g ai/ha, 600-2600 g ai/ha, 1300-3100 g ai/ha, 1500-3000 g ai/ha, 2400-3700 g ai/ha, or 340-3900 g ai/ha.

### Bentazone

In some aspects, the PDS inhibitor herbicide can comprise bentazone or an agriculturally acceptable salt or ester thereof. Bentazone, shown below, is 3-isopropyl-1H-2,1,3-benzothiadiazin-4 (3H)-one 2,2-dioxide. Its herbicidal activity is exemplified in The Pesticide Manual, Seventeenth Edition, 2016. Exemplary uses of bentazone include its use for post-emergence control of broadleaf weeds in winter and spring cereals, peanuts, maize, peas, beans, rice, and soybeans.

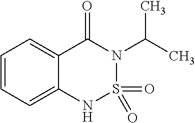 [SMILES: CC(C)N1C(=O)c2ccccc2NS1(=O)=O]

Bentazone can be applied to vegetation or an area adjacent the vegetation or applied to soil or water to prevent the emergence or growth of vegetation in an amount sufficient to induce a herbicidal effect. In some aspects, bentazone is applied to vegetation or an area adjacent the vegetation or applied to soil or water to prevent the emergence or growth of vegetation in an amount of 50 g ai/ha or more, such as 100 g ai/ha or more, 150 g ai/ha or more, 200 g ai/ha or more, 250 g ai/ha or more, 300 g ai/ha or more, 350 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, or 2100 g ai/ha or more; in an amount of 2200 g ai/ha or less, such as 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 350 g ai/ha or less, 300 g ai/ha or less, 250 g ai/ha or less, 200 g ai/ha or less, 150 g ai/ha or less, or 100 g ai/ha or less; or in an amount ranging from any of the minimum values described above to any of the maximum values described above, such as 50-2200 g ai/ha, 100-1700 g ai/ha, 300-1800 g ai/ha, 150-1900 g ai/ha, 450-2100 g ai/ha, 250-1600 g ai/ha, 700-1900 g ai/ha, or 100-2100 g ai/ha.

### Bromacil

In some aspects, the PDS inhibitor herbicide can comprise bromacil or an agriculturally acceptable salt or ester thereof. Bromacil, shown below, is 5-bromo-3-sec-butyl-6-methyluracil. Its herbicidal activity is exemplified in The Pesticide Manual, Seventeenth Edition, 2016. Exemplary uses of bromacil include its use for pre- and post-emergence control of broadleaf and grass weeds in citrus, pineapple, and total weed and brush control in non-crop lands.

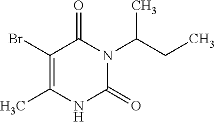

Bromacil can be applied to vegetation or an area adjacent the vegetation or applied to soil or water to prevent the emergence or growth of vegetation in an amount sufficient to induce a herbicidal effect. In some aspects, bromacil is applied to vegetation or an area adjacent the vegetation or applied to soil or water to prevent the emergence or growth of vegetation in an amount of 1500 g ai/ha or more, such as 1600 g ai/ha or more, 1750 g ai/ha or more, 2000 g ai/ha or more, 2250 g ai/ha or more, 2500 g ai/ha or more, 2750 g ai/ha or more, 3000 g ai/ha or more, 3500 g ai/ha or more, 4000 g ai/ha or more, 4500 g ai/ha or more, 5000 g ai/ha or more, 6000 g ai/ha or more, 7000 g ai/ha or more, 7500 g ai/ha or more, 8000 g ai/ha or more, 9000 g ai/ha or more, 10,000 g ai/ha or more, 11,000 g ai/ha or more, 12,000 g ai/ha or more, 13,000 g ai/ha or more, 14,000 g ai/ha or more, or 14,500 g ai/ha or more; in an amount of 15,000 g ai/ha or less, such as 14,500 g ai/ha or less, 14,000 g ai/ha or less, 13,000 g ai/ha or less, 12,000 g ai/ha or less, 11,000 g ai/ha or less, 10,000 g ai/ha or less, 9000 g ai/ha or less, 8000 g ai/ha or less, 7500 g ai/ha or less, 7000 g ai/ha or less, 6000 g ai/ha or less, 5000 g ai/ha or less, 4500 g ai/ha or less, 4000 g ai/ha or less, 3500 g ai/ha or less, 3000 g ai/ha or less, 2750 g ai/ha or less, 2500 g ai/ha or less, 2250 g ai/ha or less, 2000 g ai/ha or less, 1750 g ai/ha or less, or 1600 g ai/ha or less; or in an amount ranging from any of the minimum values described above to any of the maximum values described above, such as 1500-15,000 g ai/ha, 2500-13,000 g ai/ha, 6000-9000 g ai/ha, 8000-14,000 g ai/ha, 4500-11,000 g ai/ha, 2750-7500 g ai/ha, 3000-12,000 g ai/ha, 7000-10,000 g ai/ha, 4000-12,000 g ai/ha, or 1600-14,500 g ai/ha.

### Bromofenoxim

In some aspects, the PDS inhibitor herbicide can comprise bromofenoxim or an agriculturally acceptable salt or ester thereof. Bromofenoxim, shown below, is 3,5-dibromo-4-hydroxybenzaldehyde 2,4-dinitrophenyloxime. Its herbicidal activity is exemplified in The Pesticide Manual, Seventeenth Edition, 2016. Exemplary uses of bromofenoxim include its use for post-emergence control of many annual broadleaf weeds in cereals, maize, grass-seed crops, grassland, and new-seeded turf.

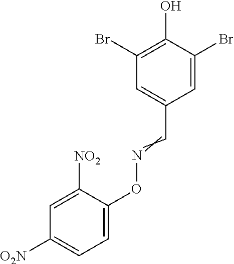 [SMILES: O=[N+]([O-])c1ccc(ON=Cc2cc(Br)c(O)c(Br)c2)c([N+](=O)[O-])c1]

Bromofenoxim can be applied to vegetation or an area adjacent the vegetation or applied to soil or water to prevent the emergence or growth of vegetation in an amount sufficient to induce a herbicidal effect. In some aspects, bromofenoxim is applied to vegetation or an area adjacent the vegetation or applied to soil or water to prevent the emergence or growth of vegetation in an amount of 1000 g ai/ha or more, such as 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1750 g ai/ha or more, 1800 g ai/ha or more, 1900 g ai/ha or more, 2000 g ai/ha or more, 2050 g ai/ha or more, 2100 g ai/ha or more, 2150 g ai/ha or more, 2200 g ai/ha or more, or 2250 g ai/ha or more; in an amount of 2300 g ai/ha or less such as 2250 g ai/ha or less, 2240 g ai/ha or less, 2200 g ai/ha or less, 2150 g ai/ha or less, 2100 g ai/ha or less, 2050 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1240 g ai/ha or less, 1200 g ai/ha or less, 1150 g ai/ha or less, 1100 g ai/ha or less, or 1050 g ai/ha or less; or in an amount ranging from any of the minimum values described above to any of the maximum values described above, such as 1000-2300 g ai/ha, 1200-1600 g ai/ha, 1450-2100 g ai/ha, 1100-1900 g ai/ha, 1700-2200 g ai/ha, 1300-2050 g ai/ha, or 1050-2200 g ai/ha.

### Bromoxynil

In some aspects, the PDS inhibitor herbicide can comprise bromoxynil or an agriculturally acceptable salt or ester thereof. Bromoxynil, shown below, is 3,5-dibromo-4-hydroxbenzonitrile. Its herbicidal activity is exemplified in The Pesticide Manual, Seventeenth Edition, 2016. Exemplary uses of bromoxynil include its use as in post-emergence control of annual broadleaf weeds in cereals, ryegrass seed crops, turf and non-crop land, maize, and sorghum.

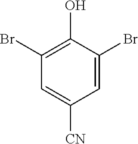 [SMILES: N#Cc1cc(Br)c(O)c(Br)c1]

Bromoxynil can be applied to vegetation or an area adjacent the vegetation or applied to soil or water to prevent the emergence or growth of vegetation in an amount sufficient to induce a herbicidal effect. In some aspects, bromoxynil is applied to vegetation or an area adjacent the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 210 g ai/ha or more, 220 g ai/ha or more, 230 g ai/ha or more, 240 g ai/ha or more, 250 g ai/ha or more, 260 g ai/ha or more, 270 g ai/ha or more, 280 g ai/ha or more, 290 g ai/ha or more, 300 g ai/ha or more, 310 g ai/ha or more, 320 g ai/ha or more, 330 g ai/ha or more, 340 g ai/ha or more, 350 g ai/ha or more, 360 g ai/ha or more, 370 g ai/ha or more, 380 g ai/ha or more, 390 g ai/ha or more, 400 g ai/ha or more, 420 g ai/ha or more, 440 g ai/ha or more, 460 g ai/ha or more, 480 g ai/ha or more, or 490 g ai/ha or more; in an amount of 500 g ai/ha or less, such as 490 g ai/ha or less, 480 g ai/ha or less, 460 g ai/ha or less, 440 g ai/ha or less, 420 g ai/ha or less, 400 g ai/ha or less, 390 g ai/ha or less, 380 g ai/ha or less, 370 g ai/ha or less, 360 g ai/ha or less, 350 g ai/ha or less, 340 g ai/ha or less, 330 g ai/ha or less, 320 g ai/ha or less, 310 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-500 g ai/ha. 80-490 g ai/ha, 60-380 g ai/ha, 200-440 g ai/ha, 130-370 g ai/ha, 260-490 g ai/ha, 320-500 g ai/ha, 400-480 g ai/ha, 240-460 g ai/ha, or 55-490 g ai/ha.

### Chlorbromuron

In some aspects, the PS-II inhibitor herbicide can comprise chlorbromuron or an agriculturally acceptable salt or ester thereof. Chlorbromuron, shown below, is 3-(4-bromo-3-chlorophenyl)-1-methoxy-1-methylurea. Its herbicidal activity is exemplified in The Pesticide Manual, Seventeenth Edition, 2016. Exemplary uses of chlorbromuron include pre-emergence use on carrots, peas, potatoes, soybeans, and sunflowers.

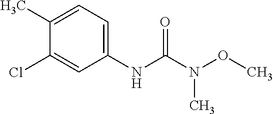

Chlorbromuron can be applied to vegetation or an area adjacent the vegetation or applied to soil or water to prevent the emergence or growth of vegetation in an amount sufficient to induce a herbicidal effect. In some aspects, chlorbromuron is applied to vegetation or an area adjacent the vegetation or applied to soil or water to prevent the emergence or growth of vegetation in an amount of 1000 g ai/ha or more, such as 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, or 2450 g ai/ha or more; in an amount of 2500 g ai/ha or less, such as 2450 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, or 1100 g ai/ha or less; or in an amount ranging from any of the minimum values described above to any of the maximum values described above, such as 1000-2500 g ai/ha, 1300-2100 g ai/ha, 1200-1900 g ai/ha, 1500-2400 g ai/ha, 1100-2200 g ai/ha, 1200-2500 g ai/ha, 1600-2300 g ai/ha, or 1100-2450 g ai/ha.

### Chloridazon

In some aspects, the PS-II inhibitor herbicide can comprise chloridazon or an agriculturally acceptable salt or ester thereof. Chloridazon, shown below, is 5-amino-4-chloro-2-phenylpyridazin-3 (2H)-one. Its herbicidal activity is exemplified in The Pesticide Manual, Seventeenth Edition, 2016. Exemplary uses of chloridazon include its use for pre- and post-emergence or pre-plant control of annual broadleaf weeds in sugar beets, fodder beets, and beetroot.

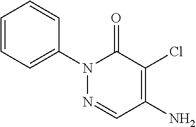 [SMILES: Nc1cnn(-c2ccccc2)c(=O)c1Cl]

Chloridazon can be applied to vegetation or an area adjacent the vegetation or applied to soil or water to prevent the emergence or growth of vegetation in an amount sufficient to induce a herbicidal effect. In some aspects, chloridazon is applied to vegetation or an area adjacent the vegetation or applied to soil or water to prevent the emergence or growth of vegetation in an amount of 1300 g ai/ha or more, such as 1350 g ai/ha or more, 1400 g ai/ha or more, 1450 g ai/ha or more, 1500 g ai/ha or more, 1550 g ai/ha or more, 1600 g ai/ha or more, 1650 g ai/ha or more, 1700 g ai/ha or more, 1750 g ai/ha or more, 1800 g ai/ha or more, 1850 g ai/ha or more, 1900 g ai/ha or more, 1950 g ai/ha or more, 2000 g ai/ha or more, 2050 g ai/ha or more, 2100 g ai/ha or more, 2150 g ai/ha or more, 2200 g ai/ha or more, 2240 g ai/ha or more, 2250 g ai/ha or more, 2300 g ai/ha or more, 2350 g ai/ha or more, 2400 g ai/ha or more, 2450 g ai/ha or more, 2500 g ai/ha or more, 2550 g ai/ha or more, 2600 g ai/ha or more, 2650 g ai/ha or more, 2700 g ai/ha or more, 2750 g ai/ha or more, 2800 g ai/ha or more, 2850 g ai/ha or more, 2900 g ai/ha or more, 2950 g ai/ha or more, 3000 g ai/ha or more, 3050 g ai/ha or more, 3100 g ai/ha or more, 3150 g ai/ha or more, or 3200 g ai/ha or more; in an amount of 3250 g ai/ha or less, such as 3200 g ai/ha or less, 3150 g ai/ha or less, 3100 g ai/ha or less, 3050 g ai/ha or less, 3000 g ai/ha or less, 2950 g ai/ha or less, 2900 g ai/ha or less, 2850 g ai/ha or less, 2800 g ai/ha or less, 2750 g ai/ha or less, 2700 g ai/ha or less, 2650 g ai/ha or less, 2600 g ai/ha or less, 2550 g ai/ha or less, 2500 g ai/ha or less, 2450 g ai/ha or less, 2400 g ai/ha or less, 2350 g ai/ha or less, 2300 g ai/ha or less, 2250 g ai/ha or less, 2200 g ai/ha or less, 2150 g ai/ha or less, 2100 g ai/ha or less, 2050 g ai/ha or less, 2000 g ai/ha or less, 1950 g ai/ha or less, 1900 g ai/ha or less, 1850 g ai/ha or less, 1800 g ai/ha or less, 1750 g ai/ha or less, 1700 g ai/ha or less, 1650 g ai/ha or less, 1600 g ai/ha or less, 1550 g ai/ha or less, 1500 g ai/ha or less, 1450 g ai/ha or less, 1400 g ai/ha or less, or 1350 g ai/ha or less; or in an amount ranging from any of the minimum values described above to any of the maximum values described above, such as 1300-3250 g ai/ha, 1500-2450 g ai/ha, 1400-2800 g ai/ha, 2050-3100 g ai/ha, 1900-2950 g ai/ha, 2000-2600 g ai/ha, 2400-3000 g ai/ha, 2250-3050 g ai/ha, or 1350-3200 g ai/ha.

### Chlorotoluron

In some aspects, the PS-II inhibitor herbicide can comprise chlorotoluron or an agriculturally acceptable salt or ester thereof. Chlorotoluron, shown below, is 3-(3-chloro-p-tolyl)-1,1-dimethylurea. Its herbicidal activity is exemplified in the online edition of The Pesticide Manual. Exemplary uses of chlorotoluron include its use for pre- and post-emergence control of many broadleaf and grass weeds in winter cereals.

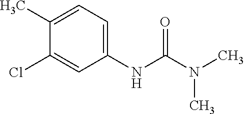

Chlorotoluron can be applied to vegetation or an area adjacent the vegetation or applied to soil or water to prevent the emergence or growth of vegetation in an amount sufficient to induce a herbicidal effect. In some aspects, chlorotoluron is applied to vegetation or an area adjacent the vegetation or applied to soil or water to prevent the emergence or growth of vegetation in an amount of 1500 g ai/ha or more, such as 1550 g ai/ha or more, 1600 g ai/ha or more, 1650 g ai/ha or more, 1700 g ai/ha or more, 1750 g ai/ha or more, 1800 g ai/ha or more, 1850 g ai/ha or more, 1900 g ai/ha or more, 1950 g ai/ha or more, 2000 g ai/ha or more, 2050 g ai/ha or more, 2100 g ai/ha or more, 2150 g ai/ha or more, 2200 g ai/ha or more, 2240 g ai/ha or more, 2250 g ai/ha or more, 2300 g ai/ha or more, 2350 g ai/ha or more, 2400 g ai/ha or more, 2450 g ai/ha or more, 2500 g ai/ha or more, 2550 g ai/ha or more, 2600 g ai/ha or more, 2650 g ai/ha or more, 2700 g ai/ha or more, 2750 g ai/ha or more, 2800 g ai/ha or more, 2850 g ai/ha or more, 2900 g ai/ha or more, or 2950 g ai/ha or more; in an amount of 3000 g ai/ha or less, such as 2950 g ai/ha or less, 2900 g ai/ha or less, 2850 g ai/ha or less, 2800 g ai/ha or less, 2750 g ai/ha or less, 2700 g ai/ha or less, 2650 g ai/ha or less, 2600 g ai/ha or less, 2550 g ai/ha or less, 2500 g ai/ha or less, 2450 g ai/ha or less, 2400 g ai/ha or less, 2350 g ai/ha or less, 2300 g ai/ha or less, 2250 g ai/ha or less, 2200 g ai/ha or less, 2150 g ai/ha or less, 2100 g ai/ha or less, 2050 g ai/ha or less, 2000 g ai/ha or less, 1950 g ai/ha or less, 1900 g ai/ha or less, 1850 g ai/ha or less, 1800 g ai/ha or less, 1750 g ai/ha or less, 1700 g ai/ha or less, 1650 g ai/ha or less, 1600 g ai/ha or less, or 1550 g ai/ha or less; or in an amount ranging from any of the minimum values described above to any of the maximum values described above, such as 1500-3000 g ai/ha, 1650-2750 g ai/ha, 1700-2300 g ai/ha, 2100-2800 g ai/ha, 2050-2600 g ai/ha, 2200-2950 g ai/ha, 1850-2400 g ai/ha, or 1550-2950 g ai/ha.

### Chloroxuron

In some aspects, the PS-II inhibitor herbicide can comprise chloroxuron or an agriculturally acceptable salt or ester thereof. Chloroxuron, shown below, is 3-[4-(4-chlorophenoxy)phenyl]-1,1-dimethylurea. Its herbicidal activity is exemplified in The Pesticide Manual, Seventeenth Edition, 2016. Exemplary uses of chloroxuron include its use for pre- and post-emergence control of annual broadleaf weeds and some grasses in peas, beans, carrots, celery, celeriac, onions, leeks, garlic, chives, fennel, parsley, dill, tomatoes, cucurbits, soybeans, strawberries, fruit trees, ornamentals, and conifers, and control of mosses in ornamental and sports turf, on paths and non-crop lands, and in glass houses. Chloroxuron may be applied to vegetation or an area adjacent the vegetation or applied to soil or water to prevent the emergence or growth of vegetation in an amount from 1 kg/ha to 4 kg/ha.

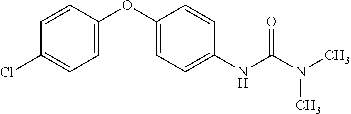 [SMILES: CN(C)C(=O)Nc1ccc(Oc2ccc(Cl)cc2)cc1]

### Cyanazine

In some aspects, the PS-II inhibitor herbicide can comprise cyanazine or an agriculturally acceptable salt or ester thereof. Cyanazine is 2-(4-chloro-6-(ethylamino)-1,3,5-triazin-2-ylamino)-2-methylpropiononitrile. Its herbicidal activity is exemplified in The Pesticide Manual, Seventeenth Edition, 2016. Exemplary uses of cyanazine include its use for pre- or post-emergence control of grass and broadleaf weeds in broad beans, maize, peas, barley, wheat, cotton, oilseed rape, forestry, potatoes, soybeans, and sugar cane.

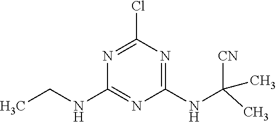 [SMILES: CCNc1nc(Cl)nc(NC(C)(C)C#N)n1]

Cyanazine can be applied to vegetation or an area adjacent the vegetation or applied to soil or water to prevent the emergence or growth of vegetation in an amount sufficient to induce a herbicidal effect. In some aspects, cyanazine is applied to vegetation or an area adjacent the vegetation or applied to soil or water to prevent the emergence or growth of vegetation in an amount of 250 g ai/ha or more, such as 260 g ai/ha or more, 270 g ai/ha or more, 280 g ai/ha or more, 290 g ai/ha or more, 300 g ai/ha or more, 310 g ai/ha or more, 320 g ai/ha or more, 330 g ai/ha or more, 340 g ai/ha or more, 350 g ai/ha or more, 360 g ai/ha or more, 370 g ai/ha or more, 380 g ai/ha or more, 390 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, or 2900 g ai/ha or more; in an amount of 3000 g ai/ha or less, such as 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 390 g ai/ha or less, 380 g ai/ha or less, 370 g ai/ha or less, 360 g ai/ha or less, 350 g ai/ha or less, 340 g ai/ha or less, 330 g ai/ha or less, 320 g ai/ha or less, 310 g ai/ha or less, 300 g ai/ha or less, 290 g ai/ha or less, 280 g ai/ha or less, 270 g ai/ha or less, or 260 g ai/ha or less; or in an amount ranging from any of the minimum values described above to any of the maximum values described above, such as 250-3000 g ai/ha, 625-1400 g ai/ha, 390-2300 g ai/ha, 1100-2100 g ai/ha, 1300-2700 g ai/ha, 1000-1900 g ai/ha, or 260-2900 g ai/ha.

### Desmedipham

In some aspects, the PS-II inhibitor herbicide can comprise desmedipham or an agriculturally acceptable salt or ester thereof. Desmedipham, shown below, is 3-ethoxycarbonylaminophenyl phenylcarbamate. Its herbicidal activity is exemplified in The Pesticide Manual, Seventeenth Edition, 2016. Exemplary uses of desmedipham include its use in post-emergence control of broadleaf weeds in beets.

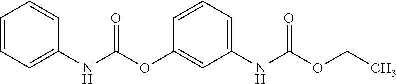 [SMILES: CCOC(=O)Nc1cccc(OC(=O)Nc2ccccc2)c1]

Desmedipham can be applied to vegetation or an area adjacent the vegetation or applied to soil or water to prevent the emergence or growth of vegetation in an amount sufficient to induce a herbicidal effect. In some aspects, desmedipham is applied to vegetation or an area adjacent the vegetation or applied to soil or water to prevent the emergence or growth of vegetation in an amount of 300 g ai/ha or more, such as 310 g ai/ha or more, 320 g ai/ha or more, 330 g ai/ha or more, 340 g ai/ha or more, 350 g ai/ha or more, 360 g ai/ha or more, 370 g ai/ha or more, 380 g ai/ha or more, 390 g ai/ha or more, 400 g ai/ha or more, 410 g ai/ha or more, 420 g ai/ha or more, 430 g ai/ha or more, 440 g ai/ha or more, 450 g ai/ha or more, 460 g ai/ha or more, 480 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, or 1200 g ai/ha or more; in an amount of 1250 g ai/ha or less, such as 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 575 g ai/ha or less, 550 g ai/ha or less, 525 g ai/ha or less, 500 g ai/ha or less, 480 g ai/ha or less, 460 g ai/ha or less, 450 g ai/ha or less, 440 g ai/ha or less, 430 g ai/ha or less, 420 g ai/ha or less, 410 g ai/ha or less, 400 g ai/ha or less, 390 g ai/ha or less, 380 g ai/ha or less, 370 g ai/ha or less, 360 g ai/ha or less, 350 g ai/ha or less, 340 g ai/ha or less, 330 g ai/ha or less, 320 g ai/ha or less, or 310 g ai/ha or less; or in an amount ranging from any of the minimum values described above to any of the maximum values described above, such as 300-1250 g ai/ha, 340-850 g ai/ha, 370-600 g ai/ha, 410-1050 g ai/ha, 440-900 g ai/ha, 450-750 g ai/ha, 500-1150 g ai/ha, 600-1200 g ai/ha, 350-950 g ai/ha, or 310-1200 g ai/ha.

### Desmetryn

In some aspects, the PS-II inhibitor herbicide can comprise desmetryn or an agriculturally acceptable salt or ester thereof. Desmetryn, shown below, is N²-isopropyl-N⁴-methyl-6-methylthio-1,3,5-triazine-2,4-diamine. Its herbicidal activity is exemplified in The Pesticide Manual, Seventeenth Edition, 2016. Exemplary uses of desmetryn include its use for pre-emergence control of fat-hen and other annual broadleaf weeds and some grasses in brassicas (except cauliflower and broccoli), herbs, onions, leeks, and conifer seedbeds.

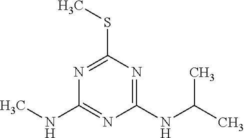

Desmetryn can be applied to vegetation or an area adjacent the vegetation or applied to soil or water to prevent the emergence or growth of vegetation in an amount sufficient to induce a herbicidal effect. In some aspects, desmetryn is applied to vegetation or an area adjacent the vegetation or applied to soil or water to prevent the emergence or growth of vegetation in an amount of 200 g ai/ha or more, such as 200 g ai/ha or more, 210 g ai/ha or more, 220 g ai/ha or more, 230 g ai/ha or more, 240 g ai/ha or more, 250 g ai/ha or more, 260 g ai/ha or more, 270 g ai/ha or more, 280 g ai/ha or more, 290 g ai/ha or more, 300 g ai/ha or more, 310 g ai/ha or more, 320 g ai/ha or more, 330 g ai/ha or more, 340 g ai/ha or more, 350 g ai/ha or more, 360 g ai/ha or more, 370 g ai/ha or more, 380 g ai/ha or more, 390 g ai/ha or more, 400 g ai/ha or more, 420 g ai/ha or more, 440 g ai/ha or more, 450 g ai/ha or more, 460 g ai/ha or more, 480 g ai/ha or more, 500 g ai/ha or more, 520 g ai/ha or more, 540 g ai/ha or more, 550 g ai/ha or more, 560 g ai/ha or more, 580 g ai/ha or more, 600 g ai/ha or more, or 625 g ai/ha or more; in an amount of 650 g ai/ha or less, such as 625 g ai/ha or less, 600 g ai/ha or less, 580 g ai/ha or less, 560 g ai/ha or less, 550 g ai/ha or less, 540 g ai/ha or less, 520 g ai/ha or less, 500 g ai/ha or less, 480 g ai/ha or less, 460 g ai/ha or less, 450 g ai/ha or less, 440 g ai/ha or less, 420 g ai/ha or less, 400 g ai/ha or less, 390 g ai/ha or less, 380 g ai/ha or less, 370 g ai/ha or less, 360 g ai/ha or less, 350 g ai/ha or less, 340 g ai/ha or less, 330 g ai/ha or less, 320 g ai/ha or less, 310 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, or 210 g ai/ha or less; or in an amount ranging from any of the minimum values described above to any of the maximum values described above, such as 200-650 g ai/ha, 220-540 g ai/ha, 260-380 g ai/ha, 250-450 g ai/ha, 300-600 g ai/ha, 440-580 g ai/ha, or 210-625 g ai/ha.

### Dimefuron

In some aspects, the PS-II inhibitor herbicide can comprise dimefuron or an agriculturally acceptable salt or ester thereof. Dimefuron, shown below, is 3-[4-(5-tert-butyl-2,3-dihydro-2-oxo-1,3,4-oxadiazol-3-yl)-3-chlorophenyl]-1,1-dimethylurea. Its herbicidal activity is exemplified in The Pesticide Manual, Seventeenth Edition, 2016. Dimefuron is used for pre- and post-emergence control of difficult annual broadleaf weeds (e.g., Stellaria media, Chamomilla, and Matricaria spp.) in beans, certain cereals, cotton, peanuts, dormant alfalfa, oilseed rape, and peas.

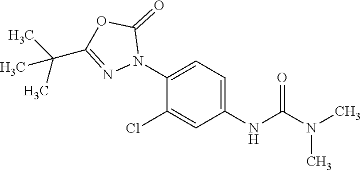 [SMILES: CN(C)C(=O)Nc1ccc(-n2nc(C(C)(C)C)oc2=O)c(Cl)c1]

Dimefuron can be applied to vegetation or an area adjacent the vegetation or applied to soil or water to prevent the emergence or growth of vegetation in an amount sufficient to induce a herbicidal effect. In some aspects, dimefuron is applied to vegetation or an area adjacent the vegetation or applied to soil or water to prevent the emergence or growth of vegetation in an amount of 200 g ai/ha or more, such as 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, or 1900 g ai/ha or more; in an amount of 2000 g ai/ha or less, such as 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, or 225 g ai/ha or less; or in an amount ranging from any of the minimum values described above to any of the maximum values described above, such as 200-2000 g ai/ha, 350-1800 g ai/ha, 425-1600 g ai/ha, 575-1100 g ai/ha, 400-1700 g ai/ha, 600-925 g ai/ha, or 225-1900 g ai/ha.

### Dimethametryn

In some aspects, the PS-II inhibitor herbicide can comprise dimethametryn or an agriculturally acceptable salt or ester thereof. Dimethametryn, shown below, is (RS)—N²-(1,2-dimethylpropyl)-N⁴-ethyl-6-(methylthio)-1,3,5-triazine-2,4-diamine. Its herbicidal activity is exemplified in The Pesticide Manual, Seventeenth Edition, 2016. Exemplary uses of dimethametryn include its use in control of annual broadleaf weeds in rice.

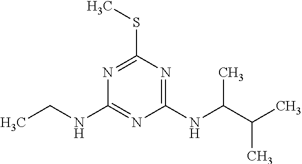 [SMILES: CCNc1nc(NC(C)C(C)C)nc(SC)n1]

Dimethametryn can be applied to vegetation or an area adjacent the vegetation or applied to soil or water to prevent the emergence or growth of vegetation in an amount sufficient to induce a herbicidal effect. In some aspects, dimethametryn is applied to vegetation or an area adjacent the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, or 145 g ai/ha or more; in an amount of 150 g ai/ha or less, such as 145 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-150 g ai/ha, 60-120 g ai/ha, 55-95 g ai/ha, 85-140 g ai/ha, 65-120 g ai/ha, 55-110 g ai/ha, 70-130 g ai/ha, or 55-145 g ai/ha.

### Diuron

In some aspects, the PS-II inhibitor herbicide can comprise diuron or an agriculturally acceptable salt or ester thereof. Diuron, shown below, is 3-(3,4-dichlorophenyl)-1,1-dimethylurea. Its herbicidal activity is exemplified in The Pesticide Manual, Seventeenth Edition, 2016. Diuron provides pre- and early post-emergence control of germinating grasses and broadleaf weeds in asparagus, tree fruit, bush fruit, citrus, vines, olives, pineapples, bananas, sugar cane, cotton, peppermint, alfalfa, forage legumes, cereals, maize, sorghum, and perennial grass seed crops, as well as total control of weeds and mosses in non-crop areas.

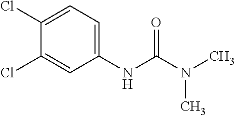

Diuron can be applied to vegetation or an area adjacent the vegetation or applied to soil or water to prevent the emergence or growth of vegetation in an amount sufficient to induce a herbicidal effect. In some aspects, diuron is applied to vegetation or an area adjacent the vegetation or applied to soil or water to prevent the emergence or growth of vegetation in an amount of 600 g ai/ha or more, such as 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3800 g ai/ha or more, 3900 g ai/ha or more, 4000 g ai/ha or more, 4100 g ai/ha or more, 4200 g ai/ha or more, 4300 g ai/ha or more, 4400 g ai/ha or more, 4500 g ai/ha or more, 4600 g ai/ha or more, 4700 g ai/ha or more, or 4750 g ai/ha or more; in an amount of 4800 g ai/ha or less, such as 4750 g ai/ha or less, 4700 g ai/ha or less, 4600 g ai/ha or less, 4500 g ai/ha or less, 4400 g ai/ha or less, 4300 g ai/ha or less, 4200 g ai/ha or less, 4100 g ai/ha or less, 4000 g ai/ha or less, 3900 g ai/ha or less, 3800 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, or 625 g ai/ha or less; or in an amount ranging from any of the minimum values described above to any of the maximum values described above, such as 600-4800 g ai/ha, 700-3900 g ai/ha, 875-2200 g ai/ha, 650-3100 g ai/ha, 950-2700 g ai/ha, 1500-2600 g ai/ha, 1300-3200 g ai/ha, 850-4000 g ai/ha, 2500-4400 g ai/ha, 1400-3200 g ai/ha, or 625-4750 g ai/ha.

### Ethidimuron

In some aspects, the PS-II inhibitor herbicide can comprise ethidimuron or an agriculturally acceptable salt or ester thereof. Ethidimuron, shown below, is 1-(5-ethylsulfonyl-1,3,4-thiadiazol-2-yl)-1,3-dimethylurea. Its herbicidal activity is exemplified in The Pesticide Manual, Seventeenth Edition, 2016. Exemplary uses of ethidimuron include its use for total herbicidal control on uncultivated land and in sugar cane plantations.

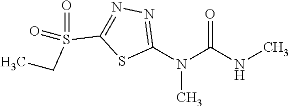 [SMILES: CCS(=O)(=O)c1nnc(N(C)C(=O)NC)s1]

Ethidimuron can be applied to vegetation or an area adjacent the vegetation or applied to soil or water to prevent the emergence or growth of vegetation in an amount sufficient to induce a herbicidal effect. In some aspects, ethidimuron is applied to vegetation or an area adjacent the vegetation or applied to soil or water to prevent the emergence or growth of vegetation in an amount of 4000 g ai/ha or more, such as 4250 g ai/ha or more, 4500 g ai/ha or more, 4750 g ai/ha or more, 5000 g ai/ha or more, 5250 g ai/ha or more, 5500 g ai/ha or more, 5750 g ai/ha or more, 6000 g ai/ha or more, 6250 g ai/ha or more, 6500 g ai/ha or more, 6750 g ai/ha or more, 7000 g ai/ha or more, or 7250 g ai/ha or more; in an amount of 7500 g ai/ha or less, such as 7250 g ai/ha or less, 7000 g ai/ha or less, 6750 g ai/ha or less, 6500 g ai/ha or less, 6250 g ai/ha or less, 6000 g ai/ha or less, 5750 g ai/ha or less, 5500 g ai/ha or less, 5250 g ai/ha or less, 5000 g ai/ha or less, 4750 g ai/ha or less, 4500 g ai/ha or less, or 4250 g ai/ha or less; or in an amount ranging from any of the minimum values described above to any of the maximum values described above, such as 4000-7500 g ai/ha, 4500-6750 g ai/ha, 5250-6500 g ai/ha, 4250-5500 g ai/ha, 5000-7250 g ai/ha, 6250-7000 g ai/ha, 5500-7500 g ai/ha, or 4250-7250 g ai/ha.

### Ethiozin

In some aspects, the PS-II inhibitor herbicide can comprise ethiozin or an agriculturally acceptable salt or ester thereof. Ethiozin, shown below, is 4-amino-6-tert-butyl-3-ethylthio-1,2,4-triazin-5 (4H)-one. Its herbicidal activity is exemplified in The Pesticide Manual, Seventeenth Edition, 2016. Exemplary uses of ethiozin include its use in pre-emergence control of grasses and some broadleaf weeds in cereals and tomatoes.

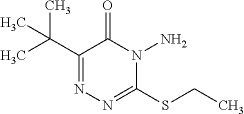

Ethiozin can be applied to vegetation or an area adjacent the vegetation or applied to soil or water to prevent the emergence or growth of vegetation in an amount sufficient to induce a herbicidal effect. In some aspects, ethiozin is applied to vegetation or an area adjacent the vegetation or applied to soil or water to prevent the emergence or growth of vegetation in an amount of 500 g ai/ha or more, such as 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, or 1700 g ai/ha or more; in an amount of 1750 g ai/ha or less, such as 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, or 525 g ai/ha or less; or in an amount ranging from any of the minimum values described above to any of the maximum values described above, such as 500-1750 g ai/ha, 550-1500 g ai/ha, 625-1600 g ai/ha, 675-1100 g ai/ha, 700-1300 g ai/ha, 600-925 g ai/ha, or 525-1700 g ai/ha.

### Fenuron

In some aspects, the PS-II inhibitor herbicide can comprise fenuron or an agriculturally acceptable salt or ester thereof. Fenuron, shown below, is 1,1-dimethyl-3-phenylurea. Its herbicidal activity is exemplified in The Pesticide Manual, Seventeenth Edition, 2016. Exemplary uses of fenuron include its use in pre-emergence control of woody plants and perennial weeds in non-crop lands.

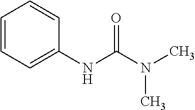

### Fluometuron

In some aspects, the PS-II inhibitor herbicide can comprise fluometuron or an agriculturally acceptable salt or ester thereof. Fluometuron, shown below, is 1,1-dimethyl-3-(α,α,α-trifluoro-m-tolyl)-urea. Its herbicidal activity is exemplified in The Pesticide Manual, Seventeenth Edition, 2016. Exemplary uses of fluometuron include its use in control of annual broadleaf and grass weeds in cotton and sugar cane.

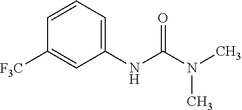 [SMILES: CN(C)C(=O)Nc1cccc(C(F)(F)F)c1]

Fluometuron can be applied to vegetation or an area adjacent the vegetation or applied to soil or water to prevent the emergence or growth of vegetation in an amount sufficient to induce a herbicidal effect. In some aspects, fluometuron is applied to vegetation or an area adjacent the vegetation or applied to soil or water to prevent the emergence or growth of vegetation in an amount of 800 g ai/ha or more, such as 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, or 1450 g ai/ha or more; in an amount of 1500 g ai/ha or less, such as 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, or 825 g ai/ha or less; or in an amount ranging from any of the minimum values described above to any of the maximum values described above, such as 800-1500 g ai/ha, 950-1300 g ai/ha, 825-1150 g ai/ha, 975-1100 g ai/ha, 1000-1400 g ai/ha, 900-1350 g ai/ha, or 825-1450 g ai/ha.

### Hexazinone

In some aspects, the PS-II inhibitor herbicide can comprise hexazinone or an agriculturally acceptable salt or ester thereof. Hexazinone, shown below, is 3-cyclohexyl-6-dimethylamino-1-methyl-1,3,5-triazine-2,4-(1H,3H)-dione. Its herbicidal activity is exemplified in The Pesticide Manual, Seventeenth Edition, 2016. Exemplary uses of hexazinone include its use in post-emergence control of annual, biennial, and most perennial weeds in alfalfa, pineapples, sugar can, coniferous species, and non-crop areas at 6,000-12,000 g/ha.

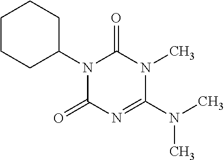 [SMILES: CN(C)c1nc(=O)n(C2CCCCC2)c(=O)n1C]

### Iodobonil

In some aspects, the PS-II inhibitor herbicide can comprise iodobonil or an agriculturally acceptable salt or ester thereof. Iodobonil, shown below, is 4-(allyloxy-carbonyloxy)-3,5-diiodobenzonitrile. Its chemical properties are given in The Pesticide Manual, Seventeenth Edition, 2016.

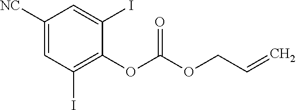 [SMILES: C=CCOC(=O)Oc1c(I)cc(C#N)cc1I]

### Ioxynil

In some aspects, the PS-II inhibitor herbicide can comprise ioxynil or an agriculturally acceptable salt or ester thereof. Ioxynil, shown below, is 4-hydroxy-3,5-diiodobenzonitrile. Its herbicidal activity is exemplified in The Pesticide Manual, Seventeenth Edition, 2016. Exemplary uses of ioxynil include its use in post-emergence control of broadleaf weeds in cereals, onions, leeks, garlic, shallots, flax, sugar cane, forage grasses, lawns, and newly-sown turf.

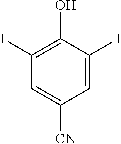 [SMILES: N#Cc1cc(I)c(O)c(I)c1]

Ioxynil can be applied to vegetation or an area adjacent the vegetation or applied to soil or water to prevent the emergence or growth of vegetation in an amount sufficient to induce a herbicidal effect. In some aspects, ioxynil is applied to vegetation or an area adjacent the vegetation or applied to soil or water to prevent the emergence or growth of vegetation in an amount of 300 g ai/ha or more, such as 310 g ai/ha or more, 320 g ai/ha or more, 330 g ai/ha or more, 340 g ai/ha or more, 350 g ai/ha or more, 360 g ai/ha or more, 370 g ai/ha or more, 380 g ai/ha or more, 390 g ai/ha or more, 400 g ai/ha or more, 420 g ai/ha or more, 440 g ai/ha or more, 450 g ai/ha or more, 460 g ai/ha or more, 480 g ai/ha or more, 500 g ai/ha or more, 520 g ai/ha or more, 540 g ai/ha or more, 550 g ai/ha or more, 560 g ai/ha or more, 580 g ai/ha or more, 600 g ai/ha or more, or 625 g ai/ha or more; in an amount of 650 g ai/ha or less, such as 625 g ai/ha or less, 600 g ai/ha or less, 580 g ai/ha or less, 560 g ai/ha or less, 550 g ai/ha or less, 540 g ai/ha or less, 520 g ai/ha or less, 500 g ai/ha or less, 480 g ai/ha or less, 460 g ai/ha or less, 450 g ai/ha or less, 440 g ai/ha or less, 420 g ai/ha or less, 400 g ai/ha or less, 390 g ai/ha or less, 380 g ai/ha or less, 370 g ai/ha or less, 360 g ai/ha or less, 350 g ai/ha or less, 340 g ai/ha or less, 330 g ai/ha or less, 320 g ai/ha or less, or 310 g ai/ha or less; or in an amount ranging from any of the minimum values described above to any of the maximum values described above, such as 300-650 g ai/ha, 320-540 g ai/ha, 360-580 g ai/ha, 350-450 g ai/ha, 310-520 g ai/ha, 440-600 g ai/ha, or 310-625 g ai/ha.

### Isocil

In some aspects, the PS-II inhibitor herbicide can comprise isocil or an agriculturally acceptable salt or ester thereof. Isocil, shown below, is 3-ethoxycarbonylaminophenyl phenylcarbamate. Its herbicidal activity is exemplified in The Pesticide Manual, Seventeenth Edition, 2016. Exemplary uses of isocil include its use as a total herbicide in control of perennial grasses and on non-cropped lands at 13-18 kg/ha.

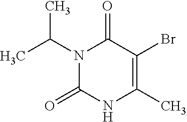

### Isomethiozin

In some aspects, the PS-II inhibitor herbicide can comprise isomethiozin or an agriculturally acceptable salt or ester thereof. Isomethiozin, shown below, is 6-tert-butyl-4-[(EZ)-isobutylideneamino]-3-methylthio-1,2,4-triazin-5 (4H)-one. Its herbicidal activity is exemplified in The Pesticide Manual, Seventeenth Edition, 2016. Exemplary uses of isomethiozin include its use in post-emergence control of areas with a heavy infestation of grass, and in winter wheat and spring cereals.

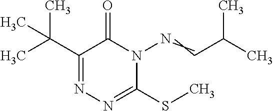 [SMILES: CSc1nnc(C(C)(C)C)c(=O)n1N=CC(C)C]

### Isoproturon

In some aspects, the PS-II inhibitor herbicide can comprise isoproturon or an agriculturally acceptable salt or ester thereof. Isoproturon, shown below, is 3-(4-isopropylphenyl)-1,1-dimethylurea. Its herbicidal activity is exemplified in The Pesticide Manual, Seventeenth Edition, 2016. Exemplary uses of isoproturon include its use in pre- and post-emergence control of annual grass weeds and many broadleaf weeds in spring and winter wheat (except durum wheat), spring and winter barley, winter rye, and triticale.

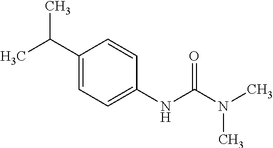

Isoproturon can be applied to vegetation or an area adjacent the vegetation or applied to soil or water to prevent the emergence or growth of vegetation in an amount sufficient to induce a herbicidal effect. In some aspects, isoproturon is applied to vegetation or an area adjacent the vegetation or applied to soil or water to prevent the emergence or growth of vegetation in an amount of 800 g ai/ha or more, such as 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, or 1450 g ai/ha or more; in an amount of 1500 g ai/ha or less, such as 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, or 825 g ai/ha or less; or in an amount ranging from any of the minimum values described above to any of the maximum values described above, such as 800-1500 g ai/ha, 950-1300 g ai/ha, 825-1150 g ai/ha, 975-1100 g ai/ha, 1000-1400 g ai/ha, 900-1350 g ai/ha, or 825-1450 g ai/ha.

### Isouron

In some aspects, the PS-II inhibitor herbicide can comprise isouron or an agriculturally acceptable salt or ester thereof. Isouron, shown below, is 3-(5-tert-butylisoxazol-3-yl)-1,1-dimethylurea. Its herbicidal activity is exemplified in The Pesticide Manual, Seventeenth Edition, 2016. Exemplary uses of isouron include its use in pre- and post-emergence control of broadleaf and grass weeds in sugar cane and pineapples.

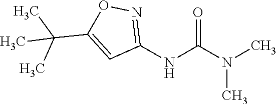

Isouron can be applied to vegetation or an area adjacent the vegetation or applied to soil or water to prevent the emergence or growth of vegetation in an amount sufficient to induce a herbicidal effect. In some aspects, isouron is applied to vegetation or an area adjacent the vegetation or applied to soil or water to prevent the emergence or growth of vegetation in an amount of 400 g ai/ha or more, such as 425 g ai/ha or more, 450 g ai/ha or more, 475 g ai/ha or more, 500 g ai/ha or more, 525 g ai/ha or more, 550 g ai/ha or more, 575 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, or 1450 g ai/ha or more; in an amount of 1500 g ai/ha or less, such as 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 575 g ai/ha or less, 550 g ai/ha or less, 525 g ai/ha or less, 500 g ai/ha or less, 475 g ai/ha or less, 450 g ai/ha or less, or 425 g ai/ha or less; or in an amount ranging from any of the minimum values described above to any of the maximum values described above, such as 400-1500 g ai/ha, 450-1300 g ai/ha, 525-1150 g ai/ha, 475-1050 g ai/ha, 600-1400 g ai/ha, 750-1250 g ai/ha, or 425-1450 g ai/ha.

### Karbutilate

In some aspects, the PS-II inhibitor herbicide can comprise karbutilate or an agriculturally acceptable salt or ester thereof. Karbutilate, shown below, is 3-(3,3-dimethylureido) phenyl tert-butylcarbamate. Its herbicidal activity is exemplified in The Pesticide Manual, Seventeenth Edition, 2016. Exemplary uses of karbutilate include its use in control of annual and perennial broadleaf and grass weeds and woody species in non-crop areas at up to 12,000 g/ha.

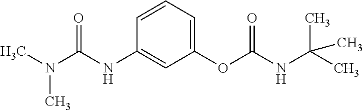 [SMILES: CN(C)C(=O)Nc1cccc(OC(=O)NC(C)(C)C)c1]

### Lenacil

In some aspects, the PS-II inhibitor herbicide can comprise lenacil or an agriculturally acceptable salt or ester thereof. Lenacil, shown below, is 3-cyclohexyl-1,5,6,7-tetrahydrocyclopentapyrimidine-2,4 (3H)-dione. Its herbicidal activity is exemplified in The

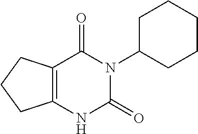 [SMILES: O=c1[nH]c2c(c(=O)n1C1CCCCC1)CCC2]

Pesticide Manual, Seventeenth Edition, 2016. Exemplary uses of lenacil include its use in pre-plant and pre-emergence control of annual grass and broadleaf weeds in sugar beets, fodder beets, beetroot, sweet potatoes, spinach, strawberries, flax, black salsify, and ornamentals.

Lenacil can be applied to vegetation or an area adjacent the vegetation or applied to soil or water to prevent the emergence or growth of vegetation in an amount sufficient to induce a herbicidal effect. In some aspects, lenacil is applied to vegetation or an area adjacent the vegetation or applied to soil or water to prevent the emergence or growth of vegetation in an amount of 100 g ai/ha or more, such as 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, or 2600 g ai/ha or more; in an amount of 2700 g ai/ha or less, such as 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, or 125 g ai/ha or less; or in an amount ranging from any of the minimum values described above to any of the maximum values described above, such as 100-2700 g ai/ha, 150-1800 g ai/ha, 225-2000 g ai/ha, 375-2300 g ai/ha, 400-1700 g ai/ha, 600-925 g ai/ha, 900-1400 g ai/ha, 1300-2500 g ai/ha, or 125-2600 g ai/ha.

### Linuron

In some aspects, the PS-II inhibitor herbicide can comprise linuron or an agriculturally acceptable salt or ester thereof. Linuron, shown below, is 3-(3,4-dichlorophenyl)-1-methoxy-1-methyurea. Its herbicidal activity is exemplified in The Pesticide Manual, Seventeenth Edition, 2016. Exemplary uses of linuron include its use in pre- and post-emergence

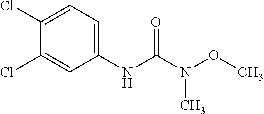

control of annual grass and broadleaf weeds in asparagus, artichokes, carrots, parsley, fennel, parsnips, herbs, celery, celeriac, onions, lees, garlic, potatoes, peas, field beans, soybeans, cereals, maize, sorghum, cotton, flax, sunflowers, sugar cane, ornamentals, established vines, bananas, cassava, coffee, tea, rice, peanuts, and other crops.

### Metamitron

In some aspects, the PS-II inhibitor herbicide can comprise metamitron or an agriculturally acceptable salt or ester thereof. Metamitron, shown below, is 4-amino-3-methyl-6-phenyl-1,2,4-triazin-5 (4H)-one. Its herbicidal activity is exemplified in The Pesticide Manual, Seventeenth Edition, 2016. Exemplary uses of metamitron include its use in pre- and post-emergence control or pre-plant control of broadleaf and grass weeds in sugar beets, fodder beets, red beets, chard, and some strawberry varieties.

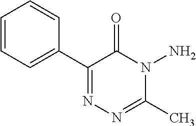

Metamitron can be applied to vegetation or an area adjacent the vegetation or applied to soil or water to prevent the emergence or growth of vegetation in an amount sufficient to induce a herbicidal effect. In some aspects, metamitron is applied to vegetation or an area adjacent the vegetation or applied to soil or water to prevent the emergence or growth of vegetation in an amount of 300 g ai/ha or more, such as 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3800 g ai/ha or more, 3900 g ai/ha or more, 4000 g ai/ha or more, or 4100 g ai/ha or more; in an amount of 4200 g ai/ha or less, such as 4100 g ai/ha or less, 4000 g ai/ha or less, 3900 g ai/ha or less, 3800 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 475 g ai/ha or less, 450 g ai/ha or less, 425 g ai/ha or less, 400 g ai/ha or less, 375 g ai/ha or less, 350 g ai/ha or less, or 325 g ai/ha or less; or in an amount ranging from any of the minimum values described above to any of the maximum values described above, such as 300-4200 g ai/ha, 400-3900 g ai/ha, 375-2200 g ai/ha, 650-3100 g ai/ha, 950-2700 g ai/ha, 500-3300 g ai/ha, 1300-4000 g ai/ha, 850-3000 g ai/ha, 2100-3600 g ai/ha, 475-3200 g ai/ha, or 325-4100 g ai/ha.

### Methabenzthiazuron

In some aspects, the PS-II inhibitor herbicide can comprise methabenzthiazuron or an agriculturally acceptable salt or ester thereof. Methabenzthiazuron, shown below, is 1-(1,3-benzothiazol-2-yl)-1,3-dimethylurea. Its herbicidal activity is exemplified in The Pesticide Manual, Seventeenth Edition, 2016. Exemplary uses of methabenzthiazuron include its use in control of broadleaf and grass weeds in cereals, legumes, maize, garlic, and onions.

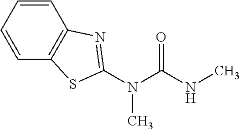

Methabenzthiazuron can be applied to vegetation or an area adjacent the vegetation or applied to soil or water to prevent the emergence or growth of vegetation in an amount sufficient to induce a herbicidal effect. In some aspects, methabenzthiazuron is applied to vegetation or an area adjacent the vegetation or applied to soil or water to prevent the emergence or growth of vegetation in an amount of 1250 g ai/ha or more, such as 1300 g ai/ha or more, 1350 g ai/ha or more, 1400 g ai/ha or more, 1450 g ai/ha or more, 1500 g ai/ha or more, 1550 g ai/ha or more, 1600 g ai/ha or more, 1650 g ai/ha or more, 1700 g ai/ha or more, 1750 g ai/ha or more, 1800 g ai/ha or more, 1850 g ai/ha or more, 1900 g ai/ha or more, 1950 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, or 2700 g ai/ha or more, 2800 g ai/ha or more, or 2900 g ai/ha or more; in an amount of 3000 g ai/ha or less, such as 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1950 g ai/ha or less, 1900 g ai/ha or less, 1850 g ai/ha or less, 1800 g ai/ha or less, 1750 g ai/ha or less, 1700 g ai/ha or less, 1650 g ai/ha or less, 1600 g ai/ha or less, 1550 g ai/ha or less, 1500 g ai/ha or less, 1450 g ai/ha or less, 1400 g ai/ha or less, 1350 g ai/ha or less, or 1300 g ai/ha or less; or in an amount ranging from any of the minimum values described above to any of the maximum values described above, such as 1250-3000 g ai/ha, 1650-2400 g ai/ha, 1450-2300 g ai/ha, 1500-2100 g ai/ha, 1300-2700 g ai/ha, 1700-1900 g ai/ha, or 1300-2900 g ai/ha.

### Methoprotryne

In some aspects, the PS-II inhibitor herbicide can comprise methoprotryne or an agriculturally acceptable salt or ester thereof. Methoprotryne, shown below, is N²-isopropyl-N⁴-(3-methoxypropyl)-6-methylthio-1,3,5-triazine-2,4-diamine. Its herbicidal activity is exemplified in The Pesticide Manual, Seventeenth Edition, 2016. Exemplary uses of methoprotryne include its use in post-emergence control of annual broadleaf weeds and grasses in winter-sown cereals.

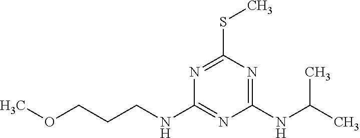 [SMILES: COCCCNc1nc(NC(C)C)nc(SC)n1]

Methoprotryne can be applied to vegetation or an area adjacent the vegetation or applied to soil or water to prevent the emergence or growth of vegetation in an amount sufficient to induce a herbicidal effect. In some aspects, methoprotryne is applied to vegetation or an area adjacent the vegetation or applied to soil or water to prevent the emergence or growth of vegetation in an amount of 1200 g ai/ha or more, such as 1250 g ai/ha or more, 1300 g ai/ha or more, 1350 g ai/ha or more, 1400 g ai/ha or more, 1450 g ai/ha or more, 1500 g ai/ha or more, 1550 g ai/ha or more, 1600 g ai/ha or more, 1650 g ai/ha or more, 1700 g ai/ha or more, 1750 g ai/ha or more, 1800 g ai/ha or more, 1850 g ai/ha or more, 1900 g ai/ha or more, 1950 g ai/ha or more, 2000 g ai/ha or more, 2050 g ai/ha or more, 2100 g ai/ha or more, 2150 g ai/ha or more, or 2200 g ai/ha or more; in an amount of 2250 g ai/ha or less, such as 2200 g ai/ha or less, 2150 g ai/ha or less, 2100 g ai/ha or less, 2050 g ai/ha or less, 2000 g ai/ha or less, 1950 g ai/ha or less, 1900 g ai/ha or less, 1850 g ai/ha or less, 1800 g ai/ha or less, 1750 g ai/ha or less, 1700 g ai/ha or less, 1 1650 g ai/ha or less, 1600 g ai/ha or less, 1550 g ai/ha or less, 1500 g ai/ha or less, 1450 g ai/ha or less, 1400 g ai/ha or less, 1350 g ai/ha or less, 1300 g ai/ha or less, or 1250 g ai/ha or less; or in an amount ranging from any of the minimum values described above to any of the maximum values described above, such as 1200-2250 g ai/ha, 1350-1800 g ai/ha, 1400-1600 g ai/ha, 1550-2100 g ai/ha, 1400-1900 g ai/ha, 1650-2050 g ai/ha, or 1250-2200 g ai/ha.

### Metobromuron

In some aspects, the PS-II inhibitor herbicide can comprise metobromuron or an agriculturally acceptable salt or ester thereof. Metobromuron, shown below, is 3-(4-bromophenyl)-1-methoxy-1-methylurea. Its herbicidal activity is exemplified in The Pesticide Manual, Seventeenth Edition, 2016. Exemplary uses of metobromuron include its use in pre-emergence control of annual broadleaf and grass weeds in beans, potatoes, tomatoes, tobacco, soybeans, maize, artichokes, and sugar beets.

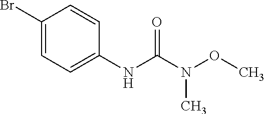

Metobromuron can be applied to vegetation or an area adjacent the vegetation or applied to soil or water to prevent the emergence or growth of vegetation in an amount sufficient to induce a herbicidal effect. In some aspects, metobromuron is applied to vegetation or an area adjacent the vegetation or applied to soil or water to prevent the emergence or growth of vegetation in an amount of 1200 g ai/ha or more, such as 1250 g ai/ha or more, 1300 g ai/ha or more, 1350 g ai/ha or more, 1400 g ai/ha or more, 1450 g ai/ha or more, 1500 g ai/ha or more, 1550 g ai/ha or more, 1600 g ai/ha or more, 1650 g ai/ha or more, 1700 g ai/ha or more, 1750 g ai/ha or more, 1800 g ai/ha or more, 1850 g ai/ha or more, 1900 g ai/ha or more, 1950 g ai/ha or more, 2000 g ai/ha or more, 2050 g ai/ha or more, 2100 g ai/ha or more, 2150 g ai/ha or more, 2200 g ai/ha or more, 2250 g ai/ha or more, 2300 g ai/ha or more, 2350 g ai/ha or more, 2400 g ai/ha or more, 2450 g ai/ha or more, or 2500 g ai/ha or more; in an amount of 2550 g ai/ha or less, such as 2500 g ai/ha or less, 2450 g ai/ha or less, 2400 g ai/ha or less, 2350 g ai/ha or less, 2300 g ai/ha or less, 2250 g ai/ha or less, 2200 g ai/ha or less, 2150 g ai/ha or less, 2100 g ai/ha or less, 2050 g ai/ha or less, 2000 g ai/ha or less, 1950 g ai/ha or less, 1900 g ai/ha or less, 1850 g ai/ha or less, 1800 g ai/ha or less, 1750 g ai/ha or less, 1700 g ai/ha or less, 1 1650 g ai/ha or less, 1600 g ai/ha or less, 1550 g ai/ha or less, 1500 g ai/ha or less, 1450 g ai/ha or less, 1400 g ai/ha or less, 1350 g ai/ha or less, 1300 g ai/ha or less, or 1250 g ai/ha or less; or in an amount ranging from any of the minimum values described above to any of the maximum values described above, such as 1200-2550 g ai/ha, 1350-2400 g ai/ha, 1450-1950 g ai/ha, 1550-2100 g ai/ha, 1600-2400 g ai/ha, 1750-2350 g ai/ha, or 1250-2500 g ai/ha.

### Metoxuron

In some aspects, the PS-II inhibitor herbicide can comprise metoxuron or an agriculturally acceptable salt or ester thereof. Metoxuron, shown below, is 3-(3-chloro-4-methoxyphenyl)-1,1-dimethylurea. Its herbicidal activity is exemplified in The Pesticide Manual, Seventeenth Edition, 2016. Exemplary uses of metoxuron include its use in pre- and post-emergence control of some grasses and annual broadleaf weeds in winter wheat, winter barley, winter rye, carrots, some varieties of spring wheat, for desiccation of potato plants, and pre-harvest defoliation in hemp, flax, and tomatoes.

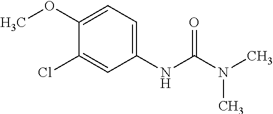

Metoxuron can be applied to vegetation or an area adjacent the vegetation or applied to soil or water to prevent the emergence or growth of vegetation in an amount sufficient to induce a herbicidal effect. In some aspects, metoxuron is applied to vegetation or an area adjacent the vegetation or applied to soil or water to prevent the emergence or growth of vegetation in an amount of 2200 g ai/ha or more, such as 2250 g ai/ha or more, 2300 g ai/ha or more, 2350 g ai/ha or more, 2400 g ai/ha or more, 2450 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3800 g ai/ha or more, 3850 g ai/ha or more, 3900 g ai/ha or more, or 3950 g ai/ha or more; in an amount of 4000 g ai/ha or less, such as 3950 g ai/ha or less, 3900 g ai/ha or less, 3850 g ai/ha or less, 3800 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2450 g ai/ha or less, 2400 g ai/ha or less, 2350 g ai/ha or less, 2300 g ai/ha or less, or 2250 g ai/ha or less; or in an amount ranging from any of the minimum values described above to any of the maximum values described above, such as 2200-4000 g ai/ha, 2300-3900 g ai/ha, 2450-3200 g ai/ha, 2600-3300 g ai/ha, 2500-3800 g ai/ha, 2350-3200 g ai/ha, 2900-4000 g ai/ha, 2200-3600 g ai/ha, 2800-3700 g ai/ha, 2400-3200 g ai/ha, or 2250-3950 g ai/ha.

### Metribuzin

In some aspects, the PS-II inhibitor herbicide can comprise metribuzin or an agriculturally acceptable salt or ester thereof. Metribuzin, shown below, is 4-amino-6-tert-butyl-3-methythio-1,2,4-triazin-5 (4H)-one. Its herbicidal activity is exemplified in The Pesticide Manual, Seventeenth Edition, 2016. Exemplary uses of metribuzin include its use in pre- and post-emergence control of grass and broadleaf weeds in soybeans, potatoes, tomatoes, sugar cane, alfalfa, asparagus, maize, and cereals.

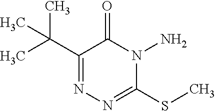

Metribuzin can be applied to vegetation or an area adjacent the vegetation or applied to soil or water to prevent the emergence or growth of vegetation in an amount sufficient to induce a herbicidal effect. In some aspects, metribuzin is applied to vegetation or an area adjacent the vegetation or applied to soil or water to prevent the emergence or growth of vegetation in an amount of 20 g ai/ha or more, such as 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, or 1450 g ai/ha or more; in an amount of 1500 g ai/ha or less, such as 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 25 g ai/ha or less; or in an amount ranging from any of the minimum values described above to any of the maximum values described above, such as 20-1500 g ai/ha, 25-800 g ai/ha, 65-200 g ai/ha, 75-1300 g ai/ha, 80-700 g ai/ha, 200-925 g ai/ha, 120-1050 g ai/ha, 150-1250 g ai/ha, or 25-1450 g ai/ha.

### Monolinuron

In some aspects, the PS-II inhibitor herbicide can comprise monolinuron or an agriculturally acceptable salt or ester thereof. Monolinuron, shown below, is 3-(4-chlorophenyl)-1-methoxy-1-methylurea. Its herbicidal activity is exemplified in The Pesticide Manual, Seventeenth Edition, 2016. Exemplary uses of monolinuron include its use in pre- and post-emergence control of annual broadleaf and some grass weeds in asparagus, berries, maize, dwarf French beans, field beans, vines, leeks, onions, potatoes, herbs, alfalfa, flowers and ornamentals, and trees.

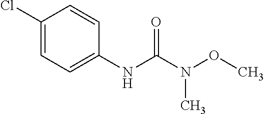

### Neburon

In some aspects, the PS-II inhibitor herbicide can comprise neburon or an agriculturally acceptable salt or ester thereof. Neburon, shown below, is 1-butyl-3-(3,4-dichlorophenyl)-1-methylurea. Its herbicidal activity is exemplified in The Pesticide Manual, Seventeenth Edition, 2016. Exemplary uses of neburon include its use in pre-emergence control of annual broadleaf and grass weeds in beans, peas, alfalfa, garlic, cereals, beets, strawberries, ornamentals, and forestry.

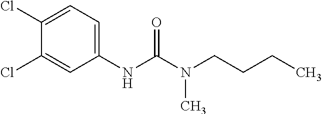 [SMILES: CCCCN(C)C(=O)Nc1ccc(Cl)c(Cl)c1]

Neburon can be applied to vegetation or an area adjacent the vegetation or applied to soil or water to prevent the emergence or growth of vegetation in an amount sufficient to induce a herbicidal effect. In some aspects, neburon is applied to vegetation or an area adjacent the vegetation or applied to soil or water to prevent the emergence or growth of vegetation in an amount of 1750 g ai/ha or more, such as 1800 g ai/ha or more, 1850 g ai/ha or more, 1900 g ai/ha or more, 1950 g ai/ha or more, 2000 g ai/ha or more, 2050 g ai/ha or more, 2100 g ai/ha or more, 2150 g ai/ha or more, 2200 g ai/ha or more, 2250 g ai/ha or more, 2300 g ai/ha or more, 2350 g ai/ha or more, 2400 g ai/ha or more, 2450 g ai/ha or more, 2500 g ai/ha or more, 2550 g ai/ha or more, 2600 g ai/ha or more, 2650 g ai/ha or more, 2700 g ai/ha or more, 2750 g ai/ha or more, 2800 g ai/ha or more, 2850 g ai/ha or more, 2900 g ai/ha or more, 2950 g ai/ha or more, 3000 g ai/ha or more, 3050 g ai/ha or more, 3100 g ai/ha or more, 3150 g ai/ha or more, or 3200 g ai/ha or more; in an amount of 3250 g ai/ha or less, such as 3200 g ai/ha or less, 3150 g ai/ha or less, 3100 g ai/ha or less, 3050 g ai/ha or less, 3000 g ai/ha or less, 2950 g ai/ha or less, 2900 g ai/ha or less, 2850 g ai/ha or less, 2800 g ai/ha or less, 2750 g ai/ha or less, 2700 g ai/ha or less, 2650 g ai/ha or less, 2600 g ai/ha or less, 2550 g ai/ha or less, 2500 g ai/ha or less, 2450 g ai/ha or less, 2400 g ai/ha or less, 2350 g ai/ha or less, 2300 g ai/ha or less, 2250 g ai/ha or less, 2200 g ai/ha or less, 2150 g ai/ha or less, 2100 g ai/ha or less, 2050 g ai/ha or less, 2000 g ai/ha or less, 1950 g ai/ha or less, 1900 g ai/ha or less, 1850 g ai/ha or less, or 1800 g ai/ha or less; or in an amount ranging from any of the minimum values described above to any of the maximum values described above, such as 1750-3250 g ai/ha, 1850-2400 g ai/ha, 1950-2700 g ai/ha, 1800-2400 g ai/ha, 2000-2800 g ai/ha, 2050-2650 g ai/ha, or 1800-3200 g ai/ha.

### Pentanochlor

In some aspects, the PS-II inhibitor herbicide can comprise pentanochlor or an agriculturally acceptable salt or ester thereof. Pentanochlor, shown below, is (RS)-3′-chloro-2-methylvalero-p-toluidide. Its herbicidal activity is exemplified in The Pesticide Manual, Seventeenth Edition, 2016. Exemplary uses of pentanochlor include its use in selective pre- and post-emergence herbicidal control in carrots, celeriac, celery, fennel, parsley, parsnips, peas, tomatoes, some flower crops, fruit trees, and ornamentals at less than 4000 g/ha.

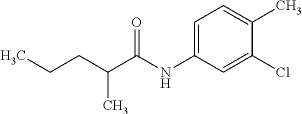 [SMILES: CCCC(C)C(=O)Nc1ccc(C)c(Cl)c1]

### Phenmedipham

In some aspects, the PS-II inhibitor herbicide can comprise phenmedipham or an agriculturally acceptable salt or ester thereof. Phenmedipham, shown below, is 3-methoxycarbonylaminophenyl 3′-methylcarbanilate. Its herbicidal activity is exemplified in The Pesticide Manual, Seventeenth Edition, 2016. Exemplary uses of phenmedipham include its use in post-emergence control of broadleaf weeds in sugar beets, strawberries, spinach, peas, chard, and red beets.

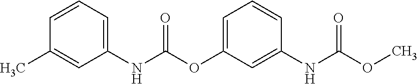

Phenmedipham can be applied to vegetation or an area adjacent the vegetation or applied to soil or water to prevent the emergence or growth of vegetation in an amount sufficient to induce a herbicidal effect. In some aspects, phenmedipham is applied to vegetation or an area adjacent the vegetation or applied to soil or water to prevent the emergence or growth of vegetation in an amount of 150 g ai/ha or more, such as 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, or 975 g ai/ha or more; in an amount of 1000 g ai/ha or less, such as 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, or 175 g ai/ha or less; or in an amount ranging from any of the minimum values described above to any of the maximum values described above, such as 150-1000 g ai/ha, 200-800 g ai/ha, 175-700 g ai/ha, 525-900 g ai/ha, 250-750 g ai/ha, 300-925 g ai/ha, 450-850 g ai/ha, 400-650 g ai/ha, or 175-975 g ai/ha.

### Prometon

In some aspects, the PS-II inhibitor herbicide can comprise prometon or an agriculturally acceptable salt or ester thereof. Prometon, shown below, is N²,N⁴-diisopropyl-6-methoxy-1,3,5-triazine-2,4-diamine. Its herbicidal activity is exemplified in The Pesticide Manual, Seventeenth Edition, 2016. Exemplary uses of prometon include its use in pre- or early post-emergence control of annual and perennial paddy weeds in direct-seeded and transplanted rice.

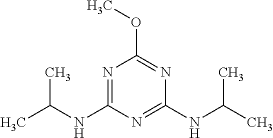 [SMILES: COc1nc(NC(C)C)nc(NC(C)C)n1]

Prometon can be applied to vegetation or an area adjacent the vegetation or applied to soil or water to prevent the emergence or growth of vegetation in an amount sufficient to induce a herbicidal effect. In some aspects, prometon is applied to vegetation or an area adjacent the vegetation or applied to soil or water to prevent the emergence or growth of vegetation in an amount of 100 g ai/ha or more, such as 125 g ai/ha or more, 150 g ai/ha or more, 175 g ai/ha or more, 200 g ai/ha or more, 225 g ai/ha or more, 250 g ai/ha or more, 275 g ai/ha or more, 300 g ai/ha or more, or 325 g ai/ha or more; in an amount of 350 g ai/ha or less, such as 325 g ai/ha or less, 300 g ai/ha or less, 275 g ai/ha or less, 250 g ai/ha or less, 225 g ai/ha or less, 200 g ai/ha or less, 175 g ai/ha or less, 150 g ai/ha or less, or 125 g ai/ha or less; or in an amount ranging from any of the minimum values described above to any of the maximum values described above, such as 100-350 g ai/ha, 150-300 g ai/ha, 225-325 g ai/ha, 175-300 g ai/ha, 200-275 g ai/ha, 150-225 g ai/ha, or 125-325 g ai/ha.

### Prometryn

In some aspects, the PS-II inhibitor herbicide can comprise prometryn or an agriculturally acceptable salt or ester thereof. Prometryn, shown below, is N²,N⁴-diisopropyl-6-methylthio-1,3,5-triazine-2,4-diamine. Its herbicidal activity is exemplified in The Pesticide Manual, Seventeenth Edition, 2016. Exemplary uses of prometryn include its use in pre- or post-emergence control of annual grass and broadleaf weeds in cotton, sunflowers, peanuts, potatoes, carrots, peas, beans, celery, and leeks.

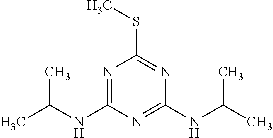 [SMILES: CSc1nc(NC(C)C)nc(NC(C)C)n1]

Prometryn can be applied to vegetation or an area adjacent the vegetation or applied to soil or water to prevent the emergence or growth of vegetation in an amount sufficient to induce a herbicidal effect. In some aspects, prometryn is applied to vegetation or an area adjacent the vegetation or applied to soil or water to prevent the emergence or growth of vegetation in an amount of 500 g ai/ha or more, such as 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1550 g ai/ha or more, 1600 g ai/ha or more, 1650 g ai/ha or more, 1700 g ai/ha or more, 1750 g ai/ha or more, 1800 g ai/ha or more, 1850 g ai/ha or more, 1900 g ai/ha or more, 1950 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, or 2700 g ai/ha or more; in an amount of 2750 g ai/ha or less, such as 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1950 g ai/ha or less, 1900 g ai/ha or less, 1850 g ai/ha or less, 1800 g ai/ha or less, 1750 g ai/ha or less, 1700 g ai/ha or less, 1650 g ai/ha or less, 1600 g ai/ha or less, 1550 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, or 550 g ai/ha or less; or in an amount ranging from any of the minimum values described above to any of the maximum values described above, such as 500-2750 g ai/ha, 650-2400 g ai/ha, 750-1800 g ai/ha, 900-2100 g ai/ha, 1300-2500 g ai/ha, 700-1700 g ai/ha, or 550-2700 g ai/ha.

### Propanil

In some aspects, the PS-II inhibitor herbicide can comprise propanil or an agriculturally acceptable salt or ester thereof. Propanil, shown below, is 3′,4′-dichloropropionanilide. Its herbicidal activity is exemplified in The Pesticide Manual, Seventeenth Edition, 2016. Exemplary uses of propanil include its use in post-emergence control of broadleaf and grass weeds in rice.

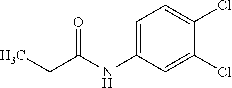

Propanil can be applied to vegetation or an area adjacent the vegetation or applied to soil or water to prevent the emergence or growth of vegetation in an amount sufficient to induce a herbicidal effect. In some aspects, propanil is applied to vegetation or an area adjacent the vegetation or applied to soil or water to prevent the emergence or growth of vegetation in an amount of 5 g ai/ha or more, such as 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 120 g ai/ha or more, 140 g ai/ha or more, 150 g ai/ha or more, 160 g ai/ha or more, 180 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3800 g ai/ha or more, or 3900 g ai/ha or more; in an amount of 4000 g ai/ha or less, such as 3900 g ai/ha or less, 3800 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 180 g ai/ha or less, 160 g ai/ha or less, 150 g ai/ha or less, 140 g ai/ha or less, 120 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, or 10 g ai/ha or less; or in an amount ranging from any of the minimum values described above to any of the maximum values described above, such as 5-4000 g ai/ha, 20-3700 g ai/ha, 250-1200 g ai/ha, 600-2300 g ai/ha, 45-800 g ai/ha, 350-3200 g ai/ha, 75-2200 g ai/ha, 850-2500 g ai/ha, 1600-3700 g ai/ha, 180-2800 g ai/ha, or 10-3950 g ai/ha.

### Propazine

In some aspects, the PS-II inhibitor herbicide can comprise propazine or an agriculturally acceptable salt or ester thereof. Propazine, shown below, is 6-chloro-N²,N⁴-diisopropyl-1,3,5-triazine-2,4-diamine. Its herbicidal activity is exemplified in The Pesticide Manual, Seventeenth Edition, 2016. Exemplary uses of propazine include its use in pre-plant, pre-, or post-emergence control of grass and broadleaf weeds in sorghum, carrots, chervil, parsley, and glasshouse ornamentals.

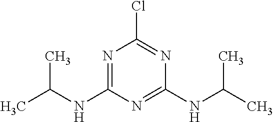

Propazine can be applied to vegetation or an area adjacent the vegetation or applied to soil or water to prevent the emergence or growth of vegetation in an amount sufficient to induce a herbicidal effect. In some aspects, propazine is applied to vegetation or an area adjacent the vegetation or applied to soil or water to prevent the emergence or growth of vegetation in an amount of 250 g ai/ha or more, such as 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, or 3400 g ai/ha or more; in an amount of 3500 g ai/ha or less, such as 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, or 275 g ai/ha or less; or in an amount ranging from any of the minimum values described above to any of the maximum values described above, such as 275-3500 g ai/ha, 300-3000 g ai/ha, 450-1200 g ai/ha, 600-2300 g ai/ha, 325-800 g ai/ha, 850-2600 g ai/ha, 750-2200 g ai/ha, 500-1500 g ai/ha, 1600-3100 g ai/ha, 900-3300 g ai/ha, or 275-3400 g ai/ha.

### Pyridafol

In some aspects, the PS-II inhibitor herbicide can comprise pyridafol or an agriculturally acceptable salt or ester thereof. Pyridafol, shown below, is 6-chloro-3-phenylpyridazin-4-ol. According to The Pesticide Manual, Seventeenth Edition, 2016, pyridafol was evaluated by Novartis Crop Protection AG.

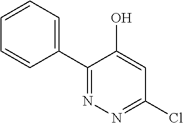 [SMILES: Oc1cc(Cl)nnc1-c1ccccc1]

### Pyridate

In some aspects, the PS-II inhibitor herbicide can comprise pyridate or an agriculturally acceptable salt or ester thereof. Pyridate, shown below, is O-6-chloro-3-phenylpyridazin-4-yl S-octyl thiocarbonate. Its herbicidal activity is exemplified in The Pesticide Manual, Seventeenth Edition, 2016. Exemplary uses of pyridate include its use in post-emergence control of annual broadleaf weeds and some grass weeds in maize, oilseed rape, cereals, rice, peanuts, and vegetables.

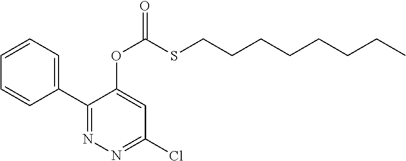 [SMILES: CCCCCCCCSC(=O)Oc1cc(Cl)nnc1-c1ccccc1]

Pyridate can be applied to vegetation or an area adjacent the vegetation or applied to soil or water to prevent the emergence or growth of vegetation in an amount sufficient to induce a herbicidal effect. In some aspects, pyridate is applied to vegetation or an area adjacent the vegetation or applied to soil or water to prevent the emergence or growth of vegetation in an amount of 100 g ai/ha or more, such as 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 210 g ai/ha or more, 220 g ai/ha or more, 230 g ai/ha or more, 240 g ai/ha or more, 250 g ai/ha or more, 260 g ai/ha or more, 270 g ai/ha or more, 280 g ai/ha or more, 290 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, or 1450 g ai/ha or more; in an amount of 1500 g ai/ha or less, such as 1450 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, or 110 g ai/ha or less; or in an amount ranging from any of the minimum values described above to any of the maximum values described above, such as 100-1500 g ai/ha, 120-700 g ai/ha, 250-1100 g ai/ha, 300-800 g ai/ha, 450-1000 g ai/ha, 350-1200 g ai/ha, 210-900 g ai/ha, 125-500 g ai/ha, 375-1400 g ai/ha, 180-600 g ai/ha, or 110-1450 g ai/ha.

### Siduron

In some aspects, the PS-II inhibitor herbicide can comprise siduron or an agriculturally acceptable salt or ester thereof. Siduron, shown below, is 1-(2-methylcyclohexyl)-3-phenylurea. Its herbicidal activity is exemplified in The Pesticide Manual, Seventeenth Edition, 2016. Exemplary uses of siduron include its use in pre-emergence control of Digitaria spp. and annual grass weeds in turf farms, grass seed production, and established turf.

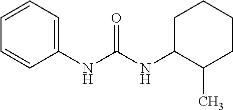 [SMILES: CC1CCCCC1NC(=O)Nc1ccccc1]

Siduron can be applied to vegetation or an area adjacent the vegetation or applied to soil or water to prevent the emergence or growth of vegetation in an amount sufficient to induce a herbicidal effect. In some aspects, siduron is applied to vegetation or an area adjacent the vegetation or applied to soil or water to prevent the emergence or growth of vegetation in an amount of 2000 g ai/ha or more, such as 2100 g ai/ha or more, 2250 g ai/ha or more, 2500 g ai/ha or more, 2750 g ai/ha or more, 3000 g ai/ha or more, 3500 g ai/ha or more, 4000 g ai/ha or more, 4500 g ai/ha or more, 5000 g ai/ha or more, 6000 g ai/ha or more, 7000 g ai/ha or more, 7500 g ai/ha or more, 8000 g ai/ha or more, 9000 g ai/ha or more, 10,000 g ai/ha or more, 11,000 g ai/ha or more, 12,000 g ai/ha or more, 13,000 g ai/ha or more, 14,000 g ai/ha or more, or 14,500 g ai/ha or more; in an amount of 15,000 g ai/ha or less, such as 14,500 g ai/ha or less, 14,000 g ai/ha or less, 13,000 g ai/ha or less, 12,000 g ai/ha or less, 11,000 g ai/ha or less, 10,000 g ai/ha or less, 9000 g ai/ha or less, 8000 g ai/ha or less, 7500 g ai/ha or less, 7000 g ai/ha or less, 6000 g ai/ha or less, 5000 g ai/ha or less, 4500 g ai/ha or less, 4000 g ai/ha or less, 3500 g ai/ha or less, 3000 g ai/ha or less, 2750 g ai/ha or less, 2500 g ai/ha or less, 2250 g ai/ha or less, or 2100 g ai/ha or less; or in an amount ranging from any of the minimum values described above to any of the maximum values described above, such as 2000-15,000 g ai/ha, 2500-13,000 g ai/ha, 6000-9000 g ai/ha, 8000-14,000 g ai/ha, 4500-11,000 g ai/ha, 2750-7500 g ai/ha, 3000-12,000 g ai/ha, 7000-10,000 g ai/ha, 4000-12,000 g ai/ha, or 2100-14,500 g ai/ha.

### Simazine

In some aspects, the PS-II inhibitor herbicide can comprise simazine or an agriculturally acceptable salt or ester thereof. Simazine, shown below, is 6-chloro-N²,N⁴-diethyl-1,3,5-triazine-2,4-diamine. Its herbicidal activity is exemplified in The Pesticide Manual, Seventeenth Edition, 2016. Exemplary uses of simazine include its use in pre-emergence control of broadleaf and annual grass weeds in pome fruit, stone fruit, bush and cane fruits, citrus, vines, strawberries, nuts, olives, pineapples, field beans, French beans, peas, maize, asparagus, hops, alfalfa, lupins, oilseed rape, artichokes, sugar cane, plantation trees, turf, and ornamentals.

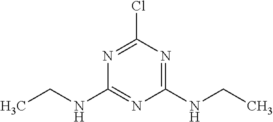

Simazine can be applied to vegetation or an area adjacent the vegetation or applied to soil or water to prevent the emergence or growth of vegetation in an amount sufficient to induce a herbicidal effect. In some aspects, simazine is applied to vegetation or an area adjacent the vegetation or applied to soil or water to prevent the emergence or growth of vegetation in an amount of 1250 g ai/ha or more, such as 1300 g ai/ha or more, 1350 g ai/ha or more, 1400 g ai/ha or more, 1450 g ai/ha or more, 1500 g ai/ha or more, 1550 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, or 3400 g ai/ha or more; in an amount of 3500 g ai/ha or less, such as 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1550 g ai/ha or less, 1500 g ai/ha or less, 1450 g ai/ha or less, 1400 g ai/ha or less, 1350 g ai/ha or less, or 1300 g ai/ha or less; or in an amount ranging from any of the minimum values described above to any of the maximum values described above, such as 1250-3500 g ai/ha, 1300-3000 g ai/ha, 1450-1900 g ai/ha, 1600-2300 g ai/ha, 1100-2800 g ai/ha, 1550-2000 g ai/ha, 2100-2600 g ai/ha, 1500-2500 g ai/ha, 1600-3100 g ai/ha, 2400-3300 g ai/ha, or 1300-3400 g ai/ha.

### Symetryn

In some aspects, the PS-II inhibitor herbicide can comprise symetryn or an agriculturally acceptable salt or ester thereof. Symetryn, shown below, is N²,N⁴-diethyl-6-methylthio-1,3,5-triazine-2,4-diamine. Its herbicidal activity is exemplified in The Pesticide Manual, Seventeenth Edition, 2016. Exemplary uses of symetryn include its use in control of broadleaf weeds in rice.

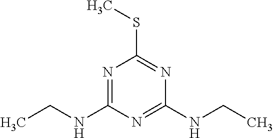

### Tebuthiuron

In some aspects, the PS-II inhibitor herbicide can comprise tebuthiuron or an agriculturally acceptable salt or ester thereof. Tebuthiuron, shown below, is 1-(5-tert-butyl-1,3,4-thiadiazol-2-yl)-1,3-dimethylurea. Its herbicidal activity is exemplified in The Pesticide Manual, Seventeenth Edition, 2016. Exemplary uses of tebuthiuron include its use in pre-emergence control of herbaceous and woody plants, annual weeds, and perennial grass and broadleaf weeds in non-crop sites, pastures, rangeland, and sugar cane.

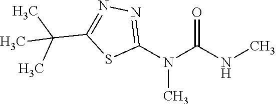

Tebuthiuron can be applied to vegetation or an area adjacent the vegetation or applied to soil or water to prevent the emergence or growth of vegetation in an amount sufficient to induce a herbicidal effect. In some aspects, tebuthiuron is applied to vegetation or an area adjacent the vegetation or applied to soil or water to prevent the emergence or growth of vegetation in an amount of 500 g ai/ha or more, such as 520 g ai/ha or more, 540 g ai/ha or more, 560 g ai/ha or more, 580 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2250 g ai/ha or more, 2500 g ai/ha or more, 2750 g ai/ha or more, 3000 g ai/ha or more, 3500 g ai/ha or more, 4000 g ai/ha or more, 4500 g ai/ha or more, 5000 g ai/ha or more, 6000 g ai/ha or more, or 6500 g ai/ha or more; in an amount of 7000 g ai/ha or less, such as 6500 g ai/ha or less, 6000 g ai/ha or less, 5000 g ai/ha or less, 4500 g ai/ha or less, 4000 g ai/ha or less, 3500 g ai/ha or less, 3000 g ai/ha or less, 2750 g ai/ha or less, 2500 g ai/ha or less, 2250 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 580 g ai/ha or less, 560 g ai/ha or less, 540 g ai/ha or less, or 520 g ai/ha or less; or in an amount ranging from any of the minimum values described above to any of the maximum values described above, such as 500-7000 g ai/ha, 2500-5000 g ai/ha, 600-900 g ai/ha, 825-1400 g ai/ha, 4500-7000 g ai/ha, 750-5000 g ai/ha, 1000-3500 g ai/ha, 975-4000 g ai/ha, 700-1200 g ai/ha, or 520-6500 g ai/ha.

### Terbacil

In some aspects, the PS-II inhibitor herbicide can comprise terbacil or an agriculturally acceptable salt or ester thereof. Terbacil, shown below, is 3-tert-butyl-5-chloro-6-methyluracil. Its herbicidal activity is exemplified in The Pesticide Manual, Seventeenth Edition, 2016. Exemplary uses of terbacil include its use in pre- and post-emergence control of annual and some perennial grass, broadleaf, and sedge weeds in apples, citrus, alfalfa, tree fruit, strawberries, blueberries, mint, and sugar cane.

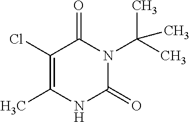

Terbacil can be applied to vegetation or an area adjacent the vegetation or applied to soil or water to prevent the emergence or growth of vegetation in an amount sufficient to induce a herbicidal effect. In some aspects, terbacil is applied to vegetation or an area adjacent the vegetation or applied to soil or water to prevent the emergence or growth of vegetation in an amount of 300 g ai/ha or more, such as 310 g ai/ha or more, 320 g ai/ha or more, 330 g ai/ha or more, 340 g ai/ha or more, 350 g ai/ha or more, 360 g ai/ha or more, 370 g ai/ha or more, 380 g ai/ha or more, 390 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1100 g ai/ha or more, 1200 g ai/ha or more, 1400 g ai/ha or more, 1600 g ai/ha or more, 1800 g ai/ha or more, 2000 g ai/ha or more, 2200 g ai/ha or more, 2400 g ai/ha or more, 2500 g ai/ha or more, 2600 g ai/ha or more, 2800 g ai/ha or more, 3000 g ai/ha or more, 3200 g ai/ha or more, 3400 g ai/ha or more, 3500 g ai/ha or more, 3600 g ai/ha or more, 3800 g ai/ha or more, 4000 g ai/ha or more, 4200 g ai/ha or more, or 4400 g ai/ha or more; in an amount of 4500 g ai/ha or less, such as 4400 g ai/ha or less, 4200 g ai/ha or less, 4000 g ai/ha or less, 3800 g ai/ha or less, 3600 g ai/ha or less, 3500 g ai/ha or less, 3400 g ai/ha or less, 3200 g ai/ha or less, 3000 g ai/ha or less, 2800 g ai/ha or less, 2600 g ai/ha or less, 2500 g ai/ha or less, 2400 g ai/ha or less, 2200 g ai/ha or less, 2000 g ai/ha or less, 1800 g ai/ha or less, 1600 g ai/ha or less, 1400 g ai/ha or less, 1200 g ai/ha or less, 1100 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 390 g ai/ha or less, 380 g ai/ha or less, 370 g ai/ha or less, 360 g ai/ha or less, 350 g ai/ha or less, 340 g ai/ha or less, 330 g ai/ha or less, 320 g ai/ha or less, or 310 g ai/ha or less; or in an amount ranging from any of the minimum values described above to any of the maximum values described above, such as 300-4500 g ai/ha, 370-4000 g ai/ha, 1400-2600 g ai/ha, 600-3800 g ai/ha, 540-2600 g ai/ha, 1100-2800 g ai/ha, 1600-2500 g ai/ha, 2000-3200 g ai/ha, 2500-4200 g ai/ha, 700-1500 g ai/ha, 350-3400 g ai/ha, or 310-4400 g ai/ha.

### Terbumeton

In some aspects, the PS-II inhibitor herbicide can comprise terbumeton or an agriculturally acceptable salt or ester thereof. Terbumeton, shown below, is N²-tert-butyl-N⁴-ethyl-6-methoxy-1,3,5-triazine-2,4-diamine. Its herbicidal activity is exemplified in The Pesticide Manual, Seventeenth Edition, 2016. Exemplary uses of terbumeton include its use in pre-emergence control of annual and perennial grass and broadleaf weeds in citrus, vineyards,

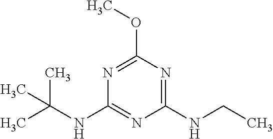 [SMILES: CCNc1nc(NC(C)(C)C)nc(OC)n1]

apples, and forestry.

Terbumeton can be applied to vegetation or an area adjacent the vegetation or applied to soil or water to prevent the emergence or growth of vegetation in an amount sufficient to induce a herbicidal effect. In some aspects, terbumeton is applied to vegetation or an area adjacent the vegetation or applied to soil or water to prevent the emergence or growth of vegetation in an amount of 2700 g ai/ha or more, such as 2750 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3600 g ai/ha or more, 3700 g ai/ha or more, 3800 g ai/ha or more, 3900 g ai/ha or more 4000 g ai/ha or more, 4100 g ai/ha or more, 4200 g ai/ha or more, 4300 g ai/ha or more, 4400 g ai/ha or more, 4500 g ai/ha or more, 4600 g ai/ha or more, 4700 g ai/ha or more, 4800 g ai/ha or more, 4900 g ai/ha or more, 5000 g ai/ha or more, 5100 g ai/ha or more, 5200 g ai/ha or more, 5300 g ai/ha or more, 5400 g ai/ha or more, 5500 g ai/ha or more, 5600 g ai/ha or more, 5700 g ai/ha or more, 5800 g ai/ha or more, 5900 g ai/ha or more, 6000 g ai/ha or more, 6100 g ai/ha or more, 6250 g ai/ha or more, 6500 g ai/ha or more, or 6750 g ai/ha or more; in an amount of 7000 g ai/ha or less, such as 6750 g ai/ha or less, 6500 g ai/ha or less, 6250 g ai/ha or less, 6100 g ai/ha or less, 6000 g ai/ha or less, 5900 g ai/ha or less, 5800 g ai/ha or less, 5700 g ai/ha or less, 5600 g ai/ha or less, 5500 g ai/ha or less, 5400 g ai/ha or less, 5300 g ai/ha or less, 5200 g ai/ha or less, 5100 g ai/ha or less, 5000 g ai/ha or less, 4900 g ai/ha or less, 4800 g ai/ha or less, 4700 g ai/ha or less, 4600 g ai/ha or less, 4500 g ai/ha or less, 4400 g ai/ha or less, 4300 g ai/ha or less, 4200 g ai/ha or less, 4100 g ai/ha or less, 4000 g ai/ha or less, 3900 g ai/ha or less, 3800 g ai/ha or less, 3700 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, or 2750 g ai/ha or less; or in an amount ranging from any of the minimum values described above to any of the maximum values described above, such as 2700-7000 g ai/ha, 3500-6000 g ai/ha, 4600-6900 g ai/ha, 2800-4400 g ai/ha, 5700-6100 g ai/ha, 3700-5000 g ai/ha, 3000-5500 g ai/ha, 2900-6300 g ai/ha, 3600-4900 g ai/ha, or 2750-6750 g ai/ha.

### Terbuthylazine

In some aspects, the PS-II inhibitor herbicide can comprise terbuthylazine or an agriculturally acceptable salt or ester thereof. Terbuthylazine, shown below, is N²-tert-butyl-6-chloro-N⁴-ethyl-1,3,5-triazine-2,4-diamine. Its herbicidal activity is exemplified in The Pesticide Manual, Seventeenth Edition, 2016. Exemplary uses of terbuthylazine include its use in pre- and post-emergence control of broadleaf and grass weeds in maize, sorghum, tree fruits, citrus, vines, coffee, oil palms, cocoa, potatoes, olives, peas, beans, sugar cane, rubber, tree nurseries, and new plantings.

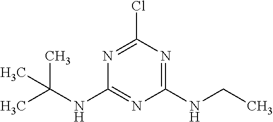

Terbuthylazine can be applied to vegetation or an area adjacent the vegetation or applied to soil or water to prevent the emergence or growth of vegetation in an amount sufficient to induce a herbicidal effect. In some aspects, terbuthylazine is applied to vegetation or an area adjacent the vegetation or applied to soil or water to prevent the emergence or growth of vegetation in an amount of 500 g ai/ha or more, such as 520 g ai/ha or more, 540 g ai/ha or more, 560 g ai/ha or more, 580 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2250 g ai/ha or more, 2500 g ai/ha or more, or 2750 g ai/ha or more; in an amount of 3000 g ai/ha or less, such as 2750 g ai/ha or less, 2500 g ai/ha or less, 2250 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 580 g ai/ha or less, 560 g ai/ha or less, 540 g ai/ha or less, or 520 g ai/ha or less; or in an amount ranging from any of the minimum values described above to any of the maximum values described above, such as 500-3000 g ai/ha, 540-2000 g ai/ha, 600-900 g ai/ha, 825-2250 g ai/ha, 700-2100 g ai/ha, 750-1700 g ai/ha, 1000-2500 g ai/ha, 975-1300 g ai/ha, 600-1900 g ai/ha, or 520-2750 g ai/ha.

### Terbutryn

In some aspects, the PS-II inhibitor herbicide can comprise terbutryn or an agriculturally acceptable salt or ester thereof. Terbutryn, shown below, is N²-tert-butyl-N⁴-ethyl-6-methylthio-1,3,5-triazine-2,4-diamine. Its herbicidal activity is exemplified in The Pesticide Manual, Seventeenth Edition, 2016. Exemplary uses of terbutryn include its use in pre-emergence control of broadleaf and grass weeds in winter cereals, beans, peas, maize, sugar cane, sunflowers, potatoes, cotton, and peanuts.

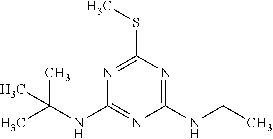 [SMILES: CCNc1nc(NC(C)(C)C)nc(SC)n1]

Terbutryn can be applied to vegetation or an area adjacent the vegetation or applied to soil or water to prevent the emergence or growth of vegetation in an amount sufficient to induce a herbicidal effect. In some aspects, terbutryn is applied to vegetation or an area adjacent the vegetation or applied to soil or water to prevent the emergence or growth of vegetation in an amount of 150 g ai/ha or more, such as 175 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, or 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more; in an amount of 3000 g ai/ha or less, such as 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, or 175 g ai/ha or less; or in an amount ranging from any of the minimum values described above to any of the maximum values described above, such as 150-3000 g ai/ha, 325-2800 g ai/ha, 575-2900 g ai/ha, 275-1300 g ai/ha, 850-1700 g ai/ha, 2000-2800 g ai/ha, 600-1050 g ai/ha, 450-1450 g ai/ha, or 175-2900 g ai/ha.

### Trietazine

In some aspects, the PS-II inhibitor herbicide can comprise trietazine or an agriculturally acceptable salt or ester thereof. Trietazine, shown below, is 6-chloro-N°, N²,N⁴-triethyl-1,3,5-triazine-2,4-diamine. Its herbicidal activity is exemplified in The Pesticide Manual, Seventeenth Edition, 2016. Exemplary uses of trietazine include its use in control of weeds in potatoes, field beans, and peas.

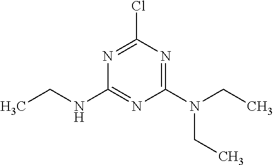

Trietazine can be applied to vegetation or an area adjacent the vegetation or applied to soil or water to prevent the emergence or growth of vegetation in an amount sufficient to induce a herbicidal effect. In some aspects, trietazine is applied to vegetation or an area adjacent the vegetation or applied to soil or water to prevent the emergence or growth of vegetation in an amount of 1500 g ai/ha or more, such as 1550 g ai/ha or more, 1600 g ai/ha or more, 1700 g ai/ha or more, 1800 g ai/ha or more, 1900 g ai/ha or more, 2000 g ai/ha or more, 2200 g ai/ha or more, 2400 g ai/ha or more, 2500 g ai/ha or more, 2600 g ai/ha or more, 2800 g ai/ha or more, 3000 g ai/ha or more, 3200 g ai/ha or more, 3400 g ai/ha or more, 3500 g ai/ha or more, 3600 g ai/ha or more, 3800 g ai/ha or more, 4000 g ai/ha or more, 4200 g ai/ha or more, 4300 g ai/ha or more, or 4400 g ai/ha or more; in an amount of 4500 g ai/ha or less, such as 4400 g ai/ha or less, 4300 g ai/ha or less, 4200 g ai/ha or less, 4000 g ai/ha or less, 3800 g ai/ha or less, 3600 g ai/ha or less, 3500 g ai/ha or less, 3400 g ai/ha or less, 3200 g ai/ha or less, 3000 g ai/ha or less, 2800 g ai/ha or less, 2600 g ai/ha or less, 2500 g ai/ha or less, 2400 g ai/ha or less, 2200 g ai/ha or less, 2000 g ai/ha or less, 1900 g ai/ha or less, 1800 g ai/ha or less, 1700 g ai/ha or less, 1600 g ai/ha or less, or 1550 g ai/ha or less; or in an amount ranging from any of the minimum values described above to any of the maximum values described above, such as 1500-4500 g ai/ha, 1600-4000 g ai/ha, 1900-2600 g ai/ha, 2600-3800 g ai/ha, 2200-3200 g ai/ha, 1900-2800 g ai/ha, 2100-2500 g ai/ha, 2000-3200 g ai/ha, 2500-4200 g ai/ha, 1500-3500 g ai/ha, or 1550-4400 g ai/ha.

### HPPD Inhibitor Herbicides

In addition to the compounds according to Formula I, the compositions can optionally include a 4-hydroxyphenyl-pyruvate dioxygenase (HPPD) inhibitor herbicide. HPPD inhibitor herbicides interfere with an oxygenase enzyme involved in the creation of energy in plants and higher order eukaryotes. Examples of HPPD inhibitors include benzobicyclon, benzofenap, bicyclopyrone, fenquinotrione, isoxachlortole, isoxaflutole, lancotrione, mesotrione, pyraquinate, pyrasulfotole, pyrazolynate, pyrazoxyfen, sulcotrione, tefuryltrione, tembotrione, tolpyralate, topramezone, or an agriculturally acceptable salt or ester thereof, and combinations thereof.

In some aspects, the composition can include a HPPD inhibitor herbicide selected from the group consisting of benzobicyclon, benzofenap, bicyclopyrone, fenquinotrione, isoxachlortole, isoxaflutole, lancotrione, mesotrione, pyrasulfotole, pyrazolynate, pyrazoxyfen, sulcotrione, tefuryltrione, tembotrione, tolpyralate, topramezone, an agriculturally acceptable salt or ester thereof, and combinations thereof.

The HPPD inhibitor herbicide or agriculturally acceptable salt or ester thereof can be applied to vegetation or an area adjacent the vegetation or applied to soil or water to prevent the emergence or growth of vegetation in an amount sufficient to induce a herbicidal effect. In some aspects, the HPPD inhibitor herbicide or agriculturally acceptable salt or ester thereof is applied to vegetation or an area adjacent the vegetation or applied to soil or water to prevent the emergence or growth of vegetation in an amount of 1 g ai/ha or more, such as 1.1 g ai/ha or more, 1.25 g ai/ha or more, 1.5 g ai/ha or more, 1.75 g ai/ha or more, 2 g ai/ha or more, 2.5 g ai/ha or more, 3 g ai/ha or more, 3.5 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 17 g ai/ha or more, 18 g ai/ha or more, 19 g ai/ha or more, 20 g ai/ha or more, 21 g ai/ha or more, 22 g ai/ha or more, 23 g ai/ha or more, 24 g ai/ha or more, 25 g ai/ha or more, 26 g ai/ha or more, 27 g ai/ha or more, 28 g ai/ha or more, 29 g ai/ha or more, 30 g ai/ha or more, 31 g ai/ha or more, 32 g ai/ha or more, 33 g ai/ha or more, 34 g ai/ha or more, 35 g ai/ha or more, 36 g ai/ha or more, 37 g ai/ha or more, 38 g ai/ha or more, 39 g ai/ha or more, 40 g ai/ha or more, 41 g ai/ha or more, 42 g ai/ha or more, 43 g ai/ha or more, 44 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 250 g ai/ha or more, 260 g ai/ha or more, 280 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2050 g ai/ha or more, 2100 g ai/ha or more, 2150 g ai/ha or more, 2200 g ai/ha or more, 2240 g ai/ha or more, 2250 g ai/ha or more, 2300 g ai/ha or more, 2350 g ai/ha or more, 2400 g ai/ha or more, 2450 g ai/ha or more, 2500 g ai/ha or more, 2600 g ai/ha or more, 2700 g ai/ha or more, 2750 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3900 g ai/ha or more, 3950 g ai/ha or more, 4000 g ai/ha or more, 4100 g ai/ha or more, 4200 g ai/ha or more, 4250 g ai/ha or more, 4300 g ai/ha or more, or 4350 g ai/ha or more, or 4400 g ai/ha or more; in an amount of 4500 g ai/ha or less, such as 4450 g ai/ha or less, 4400 g ai/ha or less, 4350 g ai/ha or less, 4300 g ai/ha or less, 4250 g ai/ha or less, 4100 g ai/ha or less, 4000 g ai/ha or less, 3950 g ai/ha or less, 3900 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2750 g ai/ha or less, 2700 g ai/ha or less, 2600 g ai/ha or less, 2500 g ai/ha or less, 2450 g ai/ha or less, 2400 g ai/ha or less, 2350 g ai/ha or less, 2300 g ai/ha or less, 2250 g ai/ha or less, 2240 g ai/ha or less, 2200 g ai/ha or less, 2150 g ai/ha or less, 2100 g ai/ha or less, 2050 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1240 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 380 g ai/ha or less, 360 g ai/ha or less, 350 g ai/ha or less, 340 g ai/ha or less, 320 g ai/ha or less, 300 g ai/ha or less, 280 g ai/ha or less, 260 g ai/ha or less, 250 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 44 g ai/ha or less, 43 g ai/ha or less, 42 g ai/ha or less, 41 g ai/ha or less, 40 g ai/ha or less, 39 g ai/ha or less, 38 g ai/ha or less, 37 g ai/ha or less, 36 g ai/ha or less, 35 g ai/ha or less, 34 g ai/ha or less, 33 g ai/ha or less, 32 g ai/ha or less, 31 g ai/ha or less, 30 g ai/ha or less, 29 g ai/ha or less, 28 g ai/ha or less, 27 g ai/ha or less, 26 g ai/ha or less, 25 g ai/ha or less, 24 g ai/ha or less, 23 g ai/ha or less, 22 g ai/ha or less, 21 g ai/ha or less, 20 g ai/ha or less, 19 g ai/ha or less, 18 g ai/ha or less, 17 g ai/ha or less, 16 ai/ha or less, 15 g ai/ha or less, 14 g ai/ha or less, 13 g ai/ha or less, 12 g ai/ha or less, 11 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3.5 g ai/ha or less, 3 g ai/ha or less, 2.5 g ai/ha or less, 2 g ai/ha or less, 1.75 g ai/ha or less, 1.5 g ai/ha or less, 1.25 g ai/ha or less, or 1.1 g ai/ha or less; or in an amount ranging from any of the minimum values described above to any of the maximum values described above, such as 1-4500 g ai/ha, 4-3900 g ai/ha, 1.75-2500 g ai/ha, 75-3100 g ai/ha, 90-900 g ai/ha, 55-4200 g ai/ha, 50-2350 g ai/ha, 80-2900 g ai/ha, 120-4100 g ai/ha, 65-2700 g ai/ha, 300-4000 g ai/ha, 1200-3600 g ai/ha, 250-2000 g ai/ha, 1.75-250 g ai/ha, 700-4250 g ai/ha, 39-1100 g ai/ha, or 1.1-4450 g ai/ha.

In some aspects, the composition contains: compounds according to Formula I; (b) a PS-II inhibitor herbicide selected from ametryne, amicarbazone, atrazine, bentazone, bromacil, bromofenoxim, bromoxynil, chlorbromuron, chloridazon, chlorotoluron, chloroxuron, cyanazine, desmedipham, desmetryn, dimefuron, dimethametryn, diuron, ethidimuron, ethiozin, fenuron, fluometuron, hexazinone, iodobonil, ioxynil, isocil, isomethiozin, isoproturon, isouron, karbutilate, lenacil, linuron, metamitron, methabenzthiazuron, methoprotryne, metobromuron, metoxuron, metribuzin, monolinuron, neburon, pentanochlor, phenmedipham, prometon, prometryn, propanil, propazine, pyridafol, pyridate, siduron, simazine, simetryne, tebuthiuron, terbacil, terbumeton, terbuthylazine, terbutryn, trietazine, or mixtures thereof; and (c) an HPPD herbicide selected from benzobicyclon, benzofenap, bicyclopyrone, fenquinotrione, isoxachlortole, isoxaflutole, lancotrione, mesotrione, pyrasulfotole, pyrazolynate, pyrazoxyfen, sulcotrione, tefuryltrione, tembotrione, tolpyralate, topramezone, an agriculturally acceptable salt or ester thereof, or combinations thereof.

### Benzobicyclon

In some aspects, the HPPD inhibitor herbicide can comprise benzobicyclon or an agriculturally acceptable salt or ester thereof. Benzobicyclon, shown below, is 3-[2-chloro-4-(methylsulfonyl)benzoyl]-4-(phenylthio) bicyclo[3.2.1]oct-3-en-2-one. Its herbicidal activity is exemplified in The Pesticide Manual, Seventeenth Edition, 2016. Exemplary uses of benzobicyclon include its use for pre- to early post-emergence control of annual and perennial paddy weeds in direct-seeded and transplanted rice.

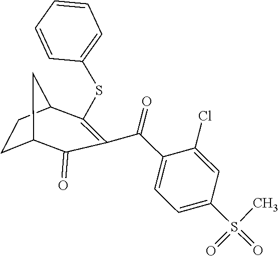 [SMILES: CS(=O)(=O)c1ccc(C(=O)C2=C(Sc3ccccc3)C3CCC(C3)C2=O)c(Cl)c1]

Benzobicyclon can be applied to vegetation or an area adjacent to the vegetation or applied to soil or water to prevent the emergence or growth of vegetation in an amount sufficient to induce a herbicidal effect. In some aspects, benzobicyclon is applied to vegetation or an area adjacent to the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, or 400 g ai/ha or more; in an amount of 401 g ai/ha or less, such as 400 g ai/ha or less, 380 g ai/ha or less, 360 g ai/ha or less, 340 g ai/ha or less, 320 g ai/ha or less, 300 g ai/ha or less, 280 g ai/ha or less, 260 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, or 50 g ai/ha or less; or in an amount ranging from any of the minimum values described above to any of the maximum values described above, such as 50-401 g ai/ha, 75-360 g ai/ha, 100-240 g ai/ha, 150-300 g ai/ha, 170-280 g ai/ha, 140-360 g ai/ha, 200-320 g ai/ha, 80-260 g ai/ha, or 55-400 g ai/ha.

### Benzofenap

In some aspects, the HPPD inhibitor herbicide can comprise benzofenap or an agriculturally acceptable salt or ester thereof. Benzofenap, shown below, is 2-[[4-(2,4-dichloro-3-methylbenzoyl)-1,3-dimethyl-1H-pyrazol-5-yl]oxy]-1-(4-methylphenyl)ethanone. Its herbicidal activity is exemplified in The Pesticide Manual, Seventeenth Edition, 2016. Exemplary uses of benzofenap include its use for control of broadleaf weeds in rice.

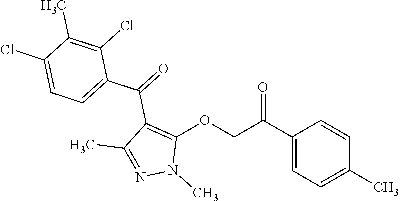 [SMILES: Cc1ccc(C(=O)COc2c(C(=O)c3ccc(Cl)c(C)c3Cl)c(C)nn2C)cc1]

Benzofenap can be applied to vegetation or an area adjacent the vegetation or applied to soil or water to prevent the emergence or growth of vegetation in an amount sufficient to induce a herbicidal effect. In some aspects, benzofenap is applied to vegetation or an area adjacent the vegetation or applied to soil or water to prevent the emergence or growth of vegetation in an amount of 300 g ai/ha or more, such as 310 g ai/ha or more, 320 g ai/ha or more, 340 g ai/ha or more, 360 g ai/ha or more, 380 g ai/ha or more, 400 g ai/ha or more, 450 g ai/ha or more, 500 g ai/ha or more, 550 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, or 1000 g ai/ha or more; in an amount of 1001 g ai/ha or less, such as 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 475 g ai/ha or less, 450 g ai/ha or less, 425 g ai/ha or less, 400 g ai/ha or less, 380 g ai/ha or less, 360 g ai/ha or less, 340 g ai/ha or less, 320 g ai/ha or less, or 310 g ai/ha or less; or in an amount ranging from any of the minimum values described above to any of the maximum values described above, such as 300-1001 g ai/ha, 450-1000 g ai/ha, 380-825 g ai/ha, 400-900 g ai/ha, 450-925 g ai/ha, 550-775 g ai/ha, 600-850 g ai/ha, 475-950 g ai/ha, 340-925 g ai/ha, 650-975 g ai/ha, 725-1000 g ai/ha, 450-850 g ai/ha, 800-950 g ai/ha, or 310-975 g ai/ha.

### Bicyclopyrone

In some aspects, the HPPD inhibitor herbicide can comprise bicyclopyrone or an agriculturally acceptable salt or ester thereof. Bicyclopyrone, shown below, is 4-hydroxy-3-[[2-[(2-methoxyethoxy)methyl]-6-(trifluoromethyl)-3-pyridinyl]carbonyl]bicyclo[3.2.1]oct-3-en-2-one. Its herbicidal activity is exemplified in The Pesticide Manual, Seventeenth Edition, 2016. Exemplary uses of bicyclopyrone include its use for pre- and early post-emergence control of broadleaf and grass weeds in maize and cereals.

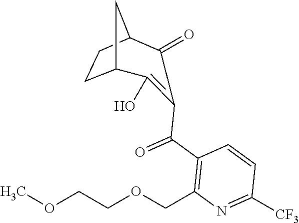 [SMILES: COCCOCc1nc(C(F)(F)F)ccc1C(=O)C1=C(O)C2CCC(C2)C1=O]

Bicyclopyrone can be applied to vegetation or an area adjacent the vegetation or applied to soil or water to prevent the emergence or growth of vegetation in an amount sufficient to induce a herbicidal effect. In some aspects, bicyclopyrone is applied to vegetation or an area adjacent the vegetation or applied to soil or water to prevent the emergence or growth of vegetation in an amount of 20 g ai/ha or more, such as 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, or 400 g ai/ha; in an amount of 401 g ai/ha or less, such as 400 g ai/ha or less, 380 g ai/ha or less, 360 g ai/ha or less, 340 g ai/ha or less, 320 g ai/ha or less, 300 g ai/ha or less, 280 g ai/ha or less, 260 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, or 20 g ai/ha or less; or in an amount ranging from any of the minimum values described above to any of the maximum values described above, such as 20-401 g ai/ha, 30-260 g ai/ha, 40-380 g ai/ha, 55-300 g ai/ha, 60-340 g ai/ha, 45-320 g ai/ha, 25-300 g ai/ha, 100-190 g ai/ha, 200-380 g ai/ha, 50-200 g ai/ha, 30-300 g ai/ha, 85-220 g ai/ha, 30-65 g ai/ha, or 25-400 g ai/ha.

### Fenquinotrione

In some aspects, the HPPD inhibitor herbicide can comprise fenquinotrione or an agriculturally acceptable salt or ester thereof. Fenquinotrione, shown below, is 2-[[8-chloro-3,4-dihydro-4-(4-methoxyphenyl)-3-oxo-2-quinoxalinyl]carbonyl]-1,3-cyclohexanedione. According to the online edition of The Pesticide Manual, fenquinotrione is a herbicide under development for use on rice.

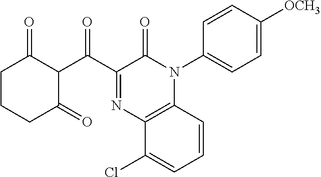 [SMILES: COc1ccc(-n2c(=O)c(C(=O)C3C(=O)CCCC3=O)nc3c(Cl)cccc32)cc1]

### Isoxachlortole

In some aspects, the HPPD inhibitor herbicide can comprise isoxachlortole or an agriculturally acceptable salt or ester thereof. Isoxachlortole, shown below, is [4-chloro-2-(methylsulfonyl)phenyl](5-cyclopropyl-4-isoxazolyl) methanone. According to the online edition of The Pesticide Manual, isoxachlortole is a herbicide that was evaluated by Rhône-Poulenc.

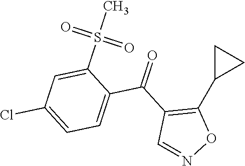 [SMILES: CS(=O)(=O)c1cc(Cl)ccc1C(=O)c1cnoc1C1CC1]

### Isoxaflutole

In some aspects, the HPPD inhibitor herbicide can comprise isoxaflutole or an agriculturally acceptable salt or ester thereof. Isoxaflutole, shown below, is (5-cyclopropyl-4-isoxazolyl) [2-(methylsulfonyl)-4-(trifluoromethyl)phenyl] methanone. Its herbicidal activity is exemplified in The Pesticide Manual. Exemplary uses of isoxaflutole include its use for pre-emergence or pre-plant control of grass and broadleaf weed in maize and sugar cane.

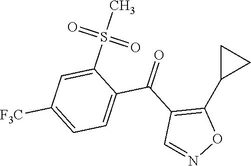 [SMILES: CS(=O)(=O)c1cc(C(F)(F)F)ccc1C(=O)c1cnoc1C1CC1]

Isoxaflutole can be applied to vegetation or an area adjacent the vegetation or applied to soil or water to prevent the emergence or growth of vegetation in an amount sufficient to induce a herbicidal effect. In some aspects, isoxaflutole is applied to vegetation or an area adjacent the vegetation or applied to soil or water to prevent the emergence or growth of vegetation in an amount of 20 g ai/ha or more, such as 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, or 300 g ai/ha or more; in an amount of 301 g ai/ha or less, such as 300 g ai/ha or less, 280 g ai/ha or less, 260 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 25 g ai/ha or less; or in an amount ranging from any of the minimum values described above to any of the maximum values described above, such as 20-301 g ai/ha, 30-300 g ai/ha, 40-170 g ai/ha, 25-260 g ai/ha, 60-120 g ai/ha, 70-190 g ai/ha, 75-240 g ai/ha, 35-200 g ai/ha, 65-220 g ai/ha, 85-140 g ai/ha, 100-300 g ai/ha, 150-280 g ai/ha, 200-301 g ai/ha, 130-200 g ai/ha, 25-100 g ai/ha, 70-90 g ai/ha, or 25-300 g ai/ha.

### Lancotrione

In some aspects, the HPPD inhibitor herbicide can comprise lancotrione or an agriculturally acceptable salt or ester thereof. Lancotrione, shown below, is 2-[2-chloro-3-[2-(1,3-dioxolan-2-yl)ethoxy]-4-(methylsulfonyl)benzoyl]-3-hydroxy-2-cyclohexen-1-one. According to the online edition of The Pesticide Manual, lancotrione is a herbicide under development by Ishihara Sangyo Kaisha, Ltd.

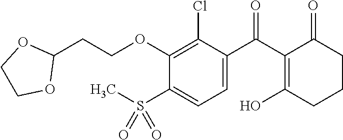 [SMILES: CS(=O)(=O)c1ccc(C(=O)C2=C(O)CCCC2=O)c(Cl)c1OCCC1OCCO1]

### Mesotrione

In some aspects, the HPPD inhibitor herbicide can comprise mesotrione or an agriculturally acceptable salt or ester thereof. Mesotrione, shown below, is 2-[4-(methylsulfonyl)-2-nitrobenzoyl]-1,3-cyclohexanedione. Its herbicidal activity is exemplified in The Pesticide Manual. Exemplary uses of mesotrione include its use for pre- and post-emergence control of broadleaf weeds and some grass weeds in maize.

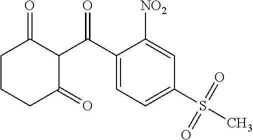 [SMILES: CS(=O)(=O)c1ccc(C(=O)C2C(=O)CCCC2=O)c([N+](=O)[O-])c1]

Mesotrione can be applied to vegetation or an area adjacent the vegetation or applied to soil or water to prevent the emergence or growth of vegetation in an amount sufficient to induce a herbicidal effect. In some aspects, mesotrione is applied to vegetation or an area adjacent the vegetation or applied to soil or water to prevent the emergence or growth of vegetation in an amount of 20 g ai/ha or more, such as 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 220 g ai/ha or more, 225 g ai/ha or more, 240 g ai/ha or more, 250 g ai/ha or more, 260 g ai/ha or more, 275 g ai/ha or more, 280 g ai/ha or more, or 300 g ai/ha or more; in an amount of 301 g ai/ha or less, such as 300 g ai/ha or less, 280 g ai/ha or less, 275 g ai/ha or less, 260 g ai/ha or less, 250 g ai/ha or less, 240 g ai/ha or less, 225 g ai/ha or less, 220 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 125 g ai/ha or less, 130 g ai/ha or less, 120 g ai/ha or less, 110 g ai/ha or less, 100 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 25 g ai/ha or less; or in an amount ranging from any of the minimum values described above to any of the maximum values described above, such as 20-301 g ai/ha, 30-275 g ai/ha, 40-140 g ai/ha, 50-250 g ai/ha, 60-301 g ai/ha, 25-280 g ai/ha, 60-125 g ai/ha, 70-250 g ai/ha, 90-225 g ai/ha, 110-220 g ai/ha, 40-175 g ai/ha, 65-160 g ai/ha, 70-200 g ai/ha, 80-180 g ai/ha, 90-250 g ai/ha, 140-225 g ai/ha, 110-240 g ai/ha, 120-175 g ai/ha, 110-150 g ai/ha, 25-85 g ai/ha, or 25-300 g ai/ha.

### Pyrasulfotole

In some aspects, the HPPD inhibitor herbicide can comprise pyrasulfotole or an agriculturally acceptable salt or ester thereof. Pyrasulfotole, shown below, is (5-hydroxy-1,3-dimethyl-1H-pyrazol-4-yl) [2-(methylsulfonyl)-4-(trifluoromethyl)phenyl]methanone. Its herbicidal activity is exemplified in The Pesticide Manual. Exemplary uses of pyrasulfotole include its use in mixture with mefenpyr-diethyl and bromoxynil or MCPA ester, for post-emergence control of broadleaf weeds in cereals.

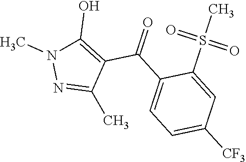 [SMILES: Cc1nn(C)c(O)c1C(=O)c1ccc(C(F)(F)F)cc1S(C)(=O)=O]

Pyrasulfotole can be applied to vegetation or an area adjacent the vegetation or applied to soil or water to prevent the emergence or growth of vegetation in an amount sufficient to induce a herbicidal effect. In some aspects, pyrasulfotole is applied to vegetation or an area adjacent the vegetation or applied to soil or water to prevent the emergence or growth of vegetation in an amount of 1 g ai/ha or more, such as 1.1 g ai/ha or more, 1.25 g ai/ha or more, 1.4 g ai/ha or more, 1.5 g ai/ha or more, 1.6 g ai/ha or more, 1.75 g ai/ha or more, 1.9 g ai/ha or more, 2 g ai/ha or more, 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.5 g ai/ha or more, 5 g ai/ha or more, 6 g ai/ha or more, 7 g ai/ha or more, 8 g ai/ha or more, 9 g ai/ha or more, 9.5 g ai/ha or more, 10 g ai/ha or more, 10.5 g ai/ha or more, 11 g ai/ha or more, 11.5 g ai/ha or more, 12 g ai/ha or more, 12.5 g ai/ha or more, 13 g ai/ha or more, 13.5 g ai/ha or more, 14 g ai/ha or more, 14.5 g ai/ha or more, 15 g ai/ha or more, 15.5 g ai/ha or more, 16 g ai/ha or more, 16.5 g ai/ha or more, 17 g ai/ha or more, 17.5 g ai/ha or more, 18 g ai/ha or more, 18.5 g ai/ha or more, 20 g ai/ha or more, 21 g ai/ha or more, 22 g ai/ha or more, 23 g ai/ha or more, 24 g ai/ha or more, 25 g ai/ha or more, 26 g ai/ha or more, 27 g ai/ha or more, 28 g ai/ha or more, 29 g ai/ha or more, 30 g ai/ha or more, 31 g ai/ha or more, 32 g ai/ha or more, 33 g ai/ha or more, 34 g ai/ha or more, 35 g ai/ha or more, 36 g ai/ha or more, 37 g ai/ha or more, 38 g ai/ha or more, 39 g ai/ha or more, 40 g ai/ha or more, 41 g ai/ha or more, 42 g ai/ha or more, 43 g ai/ha or more, 44 g ai/ha or more, 45 g ai/ha or more, or 47 g ai/ha or more; in an amount of 50 g ai/ha or less, such as 47 g ai/ha or less, 45 g ai/ha or less, 44 g ai/ha or less, 43 g ai/ha or less, 42 g ai/ha or less, 41 g ai/ha or less, 40 g ai/ha or less, 39 g ai/ha or less, 38 g ai/ha or less, 37 g ai/ha or less, 36 g ai/ha or less, 35 g ai/ha or less, 34 g ai/ha or less, 33 g ai/ha or less, 32 g ai/ha or less, 31 g ai/ha or less, 30 g ai/ha or less, 29 g ai/ha or less, 28 g ai/ha or less, 27 g ai/ha or less, 26 g ai/ha or less, 25 g ai/ha or less, 24 g ai/ha or less, 23 g ai/ha or less, 22 g ai/ha or less, 21 g ai/ha or less, 20 g ai/ha or less, 19 g ai/ha or less, 18.5 g ai/ha or less, 18 g ai/ha or less, 17.5 g ai/ha or less, 17 g ai/ha or less, 16.5 g ai/ha or less, 16 g ai/ha or less, 15.5 g ai/ha or less, 15 g ai/ha or less, 14.5 g ai/ha or less, 14 g ai/ha or less, 13.5 g ai/ha or less, 13 g ai/ha or less, 12.5 g ai/ha or less, 12 g ai/ha or less, 11.5 g ai/ha or less, 11 g ai/ha or less, 10.5 g ai/ha or less, 10 g ai/ha or less, 9.5 g ai/ha or less, 9 g ai/ha or less, 8.5 g ai/ha or less, 8 g ai/ha or less, 7.5 g ai/ha or less, 7 g ai/ha or less, 6.5 g ai/ha or less, 6 g ai/ha or less, 5.5 g ai/ha or less, 5 g ai/ha or less, 4.5 g ai/ha or less, 4 g ai/ha or less, 3.75 g ai/ha or less, 3.5 g ai/ha or less, 3.25 g ai/ha or less, 3 g ai/ha or less, 2.75 g ai/ha or less, 2.5 g ai/ha or less, 2.25 g ai/ha or less, 2 g ai/ha or less, 1.9 g ai/ha or less, 1.75 g ai/ha or less, 1.6 g ai/ha or less, 1.5 g ai/ha or less, 1.4 g ai/ha or less, 1.25 g ai/ha or less, or 1.1 g ai/ha or less; or in an amount ranging from any of the minimum values described above to any of the maximum values described above, such as 1-50 g ai/ha, 1.4-45 g ai/ha, 1.2-42 g ai/ha, 2.5-35 g ai/ha, 4-30 g ai/ha, 6.5-29 g ai/ha, 1.25-24 g ai/ha, 3.75-18.5 g ai/ha, 2.5-38 g ai/ha, 11-17 g ai/ha, 8-16 g ai/ha, 2.75-19 g ai/ha, 3-25 g ai/ha, 7-23 g ai/ha, 3.5-16 g ai/ha, 10-41 g ai/ha,

### Pyrazolynate

In some aspects, the HPPD inhibitor herbicide can comprise pyrazolynate or an agriculturally acceptable salt or ester thereof. Pyrazolynate, shown below, is (2,4-dichlorophenyl)[1,3-dimethyl-5-[[(4-methylphenyl)sulfonyl]oxy]-1H-pyrazol-4-yl]methanone. Its herbicidal activity is exemplified in The Pesticide Manual. Exemplary uses of pyrazolynate include its use to control grasses, sedges, Potamogeton distinctus, Sagittaria pygmaea, Sagittaria trifolia and Alisma canaliculatum in paddy rice.

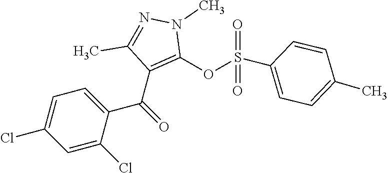 [SMILES: Cc1ccc(S(=O)(=O)Oc2c(C(=O)c3ccc(Cl)cc3Cl)c(C)nn2C)cc1]

Pyrazolynate can be applied to vegetation or an area adjacent the vegetation or applied to soil or water to prevent the emergence or growth of vegetation in an amount sufficient to induce a herbicidal effect. In some aspects, pyrazolynate is applied to vegetation or an area adjacent the vegetation or applied to soil or water to prevent the emergence or growth of vegetation in an amount of 1000 g ai/ha or more, such as 1100 g ai/ha or more, 1200 g ai/ha or more, 1400 g ai/ha or more, 1600 g ai/ha or more, 1800 g ai/ha or more, 2000 g ai/ha or more, 2200 g ai/ha or more, 2400 g ai/ha or more, 2500 g ai/ha or more, 2600 g ai/ha or more, 2800 g ai/ha or more, 3000 g ai/ha or more, 3200 g ai/ha or more, 3400 g ai/ha or more, 3500 g ai/ha or more, 3600 g ai/ha or more, 3800 g ai/ha or more, 4000 g ai/ha or more, 4200 g ai/ha or more, or 4400 g ai/ha or more; in an amount of 4500 g ai/ha or less, such as 4400 g ai/ha or less, 4200 g ai/ha or less, 4000 g ai/ha or less, 3800 g ai/ha or less, 3600 g ai/ha or less, 3500 g ai/ha or less, 3400 g ai/ha or less, 3200 g ai/ha or less, 3000 g ai/ha or less, 2800 g ai/ha or less, 2600 g ai/ha or less, 2500 g ai/ha or less, 2400 g ai/ha or less, 2200 g ai/ha or less, 2000 g ai/ha or less, 1800 g ai/ha or less, 1600 g ai/ha or less, 1400 g ai/ha or less, 1200 g ai/ha or less, or 1100 g ai/ha or less; or in an amount ranging from any of the minimum values described above to any of the maximum values described above, such as 1000-4500 g ai/ha, 2000-4000 g ai/ha, 1400-2600 g ai/ha, 1600-3800 g ai/ha, 1100-3000 g ai/ha, 1000-2800 g ai/ha, 1600-2500 g ai/ha, 2000-3200 g ai/ha, 2500-4200 g ai/ha, 1200-3500 g ai/ha, 3500-4000 g ai/ha, or 1100-4400 g ai/ha.

### Pyrazoxyfen

In some aspects, the HPPD inhibitor herbicide can comprise pyrazoxyfen or an agriculturally acceptable salt or ester thereof. Pyrazoxyfen, shown below, is 2-[[4-(2,4-dichlorobenzoyl)-1,3-dimethyl-1H-pyrazol-5-yl]oxy]-1-phenylethanone. Its herbicidal activity is exemplified in The Pesticide Manual. Exemplary uses of pyrazoxyfen include pre- or post-emergence control of annual and perennial weeds in transplanted or direct-seeded paddy rice.

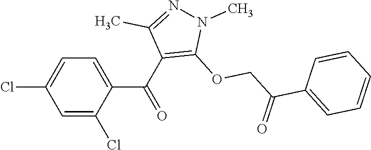 [SMILES: Cc1nn(C)c(OCC(=O)c2ccccc2)c1C(=O)c1ccc(Cl)cc1Cl]

Pyrazoxyfen can be applied to vegetation or an area adjacent the vegetation or applied to soil or water to prevent the emergence or growth of vegetation in an amount sufficient to induce a herbicidal effect. In some aspects, pyrazoxyfen is applied to vegetation or an area adjacent the vegetation or applied to soil or water to prevent the emergence or growth of vegetation in an amount of 1000 g ai/ha or more, such as 1100 g ai/ha or more, 1200 g ai/ha or more, 1400 g ai/ha or more, 1600 g ai/ha or more, 1800 g ai/ha or more, 2000 g ai/ha or more, 2200 g ai/ha or more, 2400 g ai/ha or more, 2500 g ai/ha or more, 2600 g ai/ha or more, 2800 g ai/ha or more, 3000 g ai/ha or more, 3200 g ai/ha or more, 3400 g ai/ha or more, 3500 g ai/ha or more, 3600 g ai/ha or more, 3800 g ai/ha or more, 4000 g ai/ha or more, 4200 g ai/ha or more, or 4400 g ai/ha or more; in an amount of 4500 g ai/ha or less, such as 4400 g ai/ha or less, 4200 g ai/ha or less, 4000 g ai/ha or less, 3800 g ai/ha or less, 3600 g ai/ha or less, 3500 g ai/ha or less, 3400 g ai/ha or less, 3200 g ai/ha or less, 3000 g ai/ha or less, 2800 g ai/ha or less, 2600 g ai/ha or less, 2500 g ai/ha or less, 2400 g ai/ha or less, 2200 g ai/ha or less, 2000 g ai/ha or less, 1800 g ai/ha or less, 1600 g ai/ha or less, 1400 g ai/ha or less, 1200 g ai/ha or less, or 1100 g ai/ha or less; or in an amount ranging from any of the minimum values described above to any of the maximum values described above, such as 1000-4500 g ai/ha, 1100-4000 g ai/ha, 1400-3600 g ai/ha, 1200-1800 g ai/ha, 1800-4000 g ai/ha, 2200-4500 g ai/ha, 1500-3200 g ai/ha, 2000-3500 g ai/ha, 2500-3400 g ai/ha, or 1100-4400 g ai/ha.

### Sulcotrione

In some aspects, the HPPD inhibitor herbicide can comprise sulcotrione or an agriculturally acceptable salt or ester thereof. Sulcotrione, shown below, is 2-[2-chloro-4-(methylsulfonyl)benzoyl]-1,3-cyclohexanedione. Its herbicidal activity is exemplified in The Pesticide Manual. Exemplary uses of sulcotrione include its use to control broadleaf weeds and grasses post-emergence in maize and sugar cane.

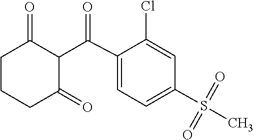 [SMILES: CS(=O)(=O)c1ccc(C(=O)C2C(=O)CCCC2=O)c(Cl)c1]

Sulcotrione can be applied to vegetation or an area adjacent the vegetation or applied to soil or water to prevent the emergence or growth of vegetation in an amount sufficient to induce a herbicidal effect. In some aspects, sulcotrione is applied to vegetation or an area adjacent the vegetation or applied to soil or water to prevent the emergence or growth of vegetation in an amount of 20 g ai/ha or more, such as 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, or 580 g ai/ha or more; in an amount of 600 g ai/ha or less, such as 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 280 g ai/ha or less, 260 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 25 g ai/ha or less; or in an amount ranging from any of the minimum values described above to any of the maximum values described above, such as 20-600 g ai/ha, 40-400 g ai/ha, 65-420 g ai/ha, 120-240 g ai/ha, 300-460 g ai/ha, 400-580 g ai/ha, 30-500 g ai/ha, 250-560 g ai/ha, 250-440 g ai/ha, 75-520 g ai/ha, 35-300 g ai/ha, 90-540 g ai/ha, 150-400 g ai/ha, 400-560 g ai/ha, 50-300 g ai/ha, 110-480 g ai/ha, 75-420 g ai/ha, 65-360 g ai/ha, 170-300 g ai/ha, 200-300 g ai/ha, 220-600 g ai/ha, or 25-580 g ai/ha.

### Tefuryltrione

In some aspects, the HPPD inhibitor herbicide can comprise tefuryltrione or an agriculturally acceptable salt or ester thereof. Tefuryltrione, shown below, is 2-[2-chloro-4-(methylsulfonyl)-3-[[(tetrahydro-2-furanyl)methoxy]methyl]benzoyl]-1,3-cyclohexandione. According to the online edition of The Pesticide Manual, tefuryltrione is a herbicide jointly developed by Bayer CropScience, Hokko Chemical, and Zen-Noh for pre- and post-emergence control of annual and perennial broadleaf weeds in rice and cereals.

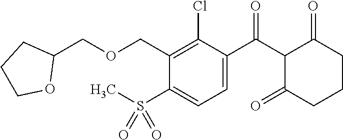 [SMILES: CS(=O)(=O)c1ccc(C(=O)C2C(=O)CCCC2=O)c(Cl)c1COCC1CCCO1]

### Tembotrione

In some aspects, the HPPD inhibitor herbicide can comprise tembotrione or an agriculturally acceptable salt or ester thereof. Tembotrione, shown below, is 2-[2-chloro-4-(methylsulfonyl)-3-[(2,2,2-trifluoroethoxy)methyl]benzoyl]-1,3-cyclohexanedione. Its herbicidal activity is exemplified in The Pesticide Manual. Exemplary uses of tembotrione include its use for post-emergence control of grass and broadleaf weed species in maize.

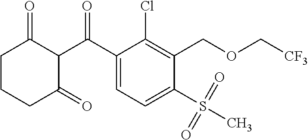 [SMILES: CS(=O)(=O)c1ccc(C(=O)C2C(=O)CCCC2=O)c(Cl)c1COCC(F)(F)F]

Tembotrione can be applied to vegetation or an area adjacent the vegetation or applied to soil or water to prevent the emergence or growth of vegetation in an amount sufficient to induce a herbicidal effect. In some aspects, tembotrione is applied to vegetation or an area adjacent the vegetation or applied to soil or water to prevent the emergence or growth of vegetation in an amount of 1 g ai/ha or more, such as 1.1 g ai/ha or more, 1.25 g ai/ha or more, 1.4 g ai/ha or more, 1.5 g ai/ha or more, 1.6 g ai/ha or more, 1.75 g ai/ha or more, 1.9 g ai/ha or more, 2 g ai/ha or more, 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.5 g ai/ha or more, 5 g ai/ha or more, 6 g ai/ha or more, 7 g ai/ha or more, 8 g ai/ha or more, 9 g ai/ha or more, 9.5 g ai/ha or more, 10 g ai/ha or more, 10.5 g ai/ha or more, 11 g ai/ha or more, 11.5 g ai/ha or more, 12 g ai/ha or more, 12.5 g ai/ha or more, 13 g ai/ha or more, 13.5 g ai/ha or more, 14 g ai/ha or more, 14.5 g ai/ha or more, 15 g ai/ha or more, 15.5 g ai/ha or more, 16 g ai/ha or more, 16.5 g ai/ha or more, 17 g ai/ha or more, 17.5 g ai/ha or more, 18 g ai/ha or more, 18.5 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, or 200 g ai/ha or more; in an amount of 200 g ai/ha or less, such as 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 19 g ai/ha or less, 18.5 g ai/ha or less, 18 g ai/ha or less, 17.5 g ai/ha or less, 17 g ai/ha or less, 16.5 g ai/ha or less, 16 g ai/ha or less, 15.5 g ai/ha or less, 15 g ai/ha or less, 14.5 g ai/ha or less, 14 g ai/ha or less, 13.5 g ai/ha or less, 13 g ai/ha or less, 12.5 g ai/ha or less, 12 g ai/ha or less, 11.5 g ai/ha or less, 11 g ai/ha or less, 10.5 g ai/ha or less, 10 g ai/ha or less, 9.5 g ai/ha or less, 9 g ai/ha or less, 8.5 g ai/ha or less, 8 g ai/ha or less, 7.5 g ai/ha or less, 7 g ai/ha or less, 6.5 g ai/ha or less, 6 g ai/ha or less, 5.5 g ai/ha or less, 5 g ai/ha or less, 4.5 g ai/ha or less, 4 g ai/ha or less, 3.75 g ai/ha or less, 3.5 g ai/ha or less, 3.25 g ai/ha or less, 3 g ai/ha or less, 2.75 g ai/ha or less, 2.5 g ai/ha or less, 2.25 g ai/ha or less, 2 g ai/ha or less, 1.9 g ai/ha or less, 1.75 g ai/ha or less, 1.6 g ai/ha or less, 1.5 g ai/ha or less, 1.4 g ai/ha or less, 1.25 g ai/ha or less, or 1.1 g ai/ha or less; or in an amount ranging from any of the minimum values described above to any of the maximum values described above, such as 1-200 g ai/ha, 1.4-75 g ai/ha, 3.5-150 g ai/ha, 10-140 g ai/ha, 6-75 g ai/ha, 8-180 g ai/ha, 1.5-25 g ai/ha, 2.5-15 g ai/ha, 2.25-10 g ai/ha, 25-120 g ai/ha, 1.9-130 g ai/ha, 20-40 g ai/ha, 30-180 g ai/ha, 45-190 g ai/ha, 3.5-70 g ai/ha, 6-130 g ai/ha, 70-90 g ai/ha, 15-110 g ai/ha, 120-140 g ai/ha, or 1.1-190 g ai/ha.

### Tolpyralate

In some aspects, the HPPD inhibitor herbicide can comprise tolpyralate or an agriculturally acceptable salt or ester thereof. Tolpyralate, shown below, is 1-[[1-ethyl-4-[3-(2-methoxyethoxy)-2-methyl-4-(methylsulfonyl)benzoyl]-1H-pyrazol-5-yl]oxy]ethyl methyl carbonate. According to the online edition of The Pesticide Manual, tefuryltrione is a herbicide developed by Ishihara Sangyo Kaisha Ltd. for pre- and post-emergence control of broadleaf and grass weeds, including Palmer amaranth, water hemp, giant ragweed, and giant foxtail in maize.

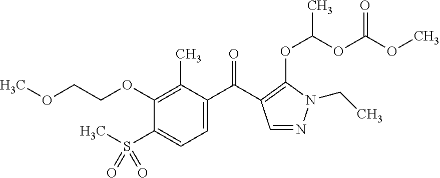

### Topramezone

In some aspects, the HPPD inhibitor herbicide can comprise topramezone or an agriculturally acceptable salt or ester thereof. Topramezone, shown below, is [3-(4,5-dihydro-3-isoxazolyl)-2-methyl-4-(methylsulfonyl)phenyl](5-hydroxy-1-methyl-1H-pyrazol-4-yl)methanone. Its herbicidal activity is exemplified in The Pesticide Manual. Exemplary uses of topramezone include its use for post-emergence weed control in maize.

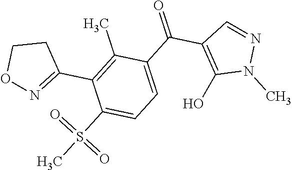 [SMILES: Cc1c(C(=O)c2cnn(C)c2O)ccc(S(C)(=O)=O)c1C1=NOCC1]

Topramezone can be applied to vegetation or an area adjacent the vegetation or applied to soil or water to prevent the emergence or growth of vegetation in an amount sufficient to induce a herbicidal effect. In some aspects, topramezone is applied to vegetation or an area adjacent the vegetation or applied to soil or water to prevent the emergence or growth of vegetation in an amount of 1 g ai/ha or more, such as 1.1 g ai/ha or more, 1.25 g ai/ha or more, 1.4 g ai/ha or more, 1.5 g ai/ha or more, 1.6 g ai/ha or more, 1.75 g ai/ha or more, 1.9 g ai/ha or more, 2 g ai/ha or more, 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.5 g ai/ha or more, 5 g ai/ha or more, 6 g ai/ha or more, 7 g ai/ha or more, 8 g ai/ha or more, 9 g ai/ha or more, 9.5 g ai/ha or more, 10 g ai/ha or more, 10.5 g ai/ha or more, 11 g ai/ha or more, 11.5 g ai/ha or more, 12 g ai/ha or more, 12.5 g ai/ha or more, 13 g ai/ha or more, 13.5 g ai/ha or more, 14 g ai/ha or more, 14.5 g ai/ha or more, 15 g ai/ha or more, 15.5 g ai/ha or more, 16 g ai/ha or more, 16.5 g ai/ha or more, 17 g ai/ha or more, 17.5 g ai/ha or more, 18 g ai/ha or more, 18.5 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, or 100 g ai/ha or more; in an amount of 100 g ai/ha or less, such as 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 19 g ai/ha or less, 18.5 g ai/ha or less, 18 g ai/ha or less, 17.5 g ai/ha or less, 17 g ai/ha or less, 16.5 g ai/ha or less, 16 g ai/ha or less, 15.5 g ai/ha or less, 15 g ai/ha or less, 14.5 g ai/ha or less, 14 g ai/ha or less, 13.5 g ai/ha or less, 13 g ai/ha or less, 12.5 g ai/ha or less, 12 g ai/ha or less, 11.5 g ai/ha or less, 11 g ai/ha or less, 10.5 g ai/ha or less, 10 g ai/ha or less, 9.5 g ai/ha or less, 9 g ai/ha or less, 8.5 g ai/ha or less, 8 g ai/ha or less, 7.5 g ai/ha or less, 7 g ai/ha or less, 6.5 g ai/ha or less, 6 g ai/ha or less, 5.5 g ai/ha or less, 5 g ai/ha or less, 4.5 g ai/ha or less, 4 g ai/ha or less, 3.75 g ai/ha or less, 3.5 g ai/ha or less, 3.25 g ai/ha or less, 3 g ai/ha or less, 2.75 g ai/ha or less, 2.5 g ai/ha or less, 2.25 g ai/ha or less, 2 g ai/ha or less, 1.9 g ai/ha or less, 1.75 g ai/ha or less, 1.6 g ai/ha or less, 1.5 g ai/ha or less, 1.4 g ai/ha or less, 1.25 g ai/ha or less, or 1.1 g ai/ha or less; or in an amount ranging from any of the minimum values described above to any of the maximum values described above, such as 1-100 g ai/ha, 2-90 g ai/ha, 4-80 g ai/ha, 2.5-75 g ai/ha, 9-60 g ai/ha, 10-55 g ai/ha, 3-25 g ai/ha, 8-15 g ai/ha, 1.5-10 g ai/ha, 5-80 g ai/ha, 6.5-45 g ai/ha, 15-85 g ai/ha, 20-90 g ai/ha, 2.5-40 g ai/ha, 3-30 g ai/ha, 9-85 g ai/ha, or 1.5-95 g ai/ha.

### HPPD Inhibitor Herbicides

In addition to the compounds according to Formula I of Formula (I), or agriculturally acceptable N-oxide, salt, or ester thereof, the compositions can include an inhibitor of 4-hydroxyphenylpyruvate dioxygenase (HPPD), an oxygenase enzyme involved in the creation of energy in plants and higher order eukaryotes. Examples of HPPD inhibitors include benzobicyclon, benzofenap, bicyclopyrone, fenquinotrione, isoxachlortole, isoxaflutole, lancotrione, mesotrione, pyrasulfotole, pyrazolynate, pyrazoxyfen, sulcotrione, tefuryltrione, tembotrione, tolpyralate, topramezone, or an agriculturally acceptable salt or esters thereof, and combinations thereof.

The HPPD inhibitor or agriculturally acceptable salt or ester thereof can be applied to vegetation or an area adjacent to the vegetation or applied to soil or water to prevent the emergence or growth of vegetation in an amount sufficient to induce a herbicidal effect. In some aspects, the HPPD inhibitor or agriculturally acceptable salt or ester thereof is applied to vegetation or an area adjacent the vegetation or applied to soil or water to prevent the emergence or growth of vegetation in an amount of 1 grams active ingredient per hectare (g ai/ha) or more, such as 1.1 g ai/ha or more, 1.25 g ai/ha or more, 1.5 g ai/ha or more, 1.75 g ai/ha or more, 2 g ai/ha or more, 2.5 g ai/ha or more, 3 g ai/ha or more, 3.5 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 11 g ai/ha or more, 12 g ai/ha or more, 13 g ai/ha or more, 14 g ai/ha or more, 15 g ai/ha or more, 16 g ai/ha or more, 17 g ai/ha or more, 18 g ai/ha or more, 19 g ai/ha or more, 20 g ai/ha or more, 21 g ai/ha or more, 22 g ai/ha or more, 23 g ai/ha or more, 24 g ai/ha or more, 25 g ai/ha or more, 26 g ai/ha or more, 27 g ai/ha or more, 28 g ai/ha or more, 29 g ai/ha or more, 30 g ai/ha or more, 31 g ai/ha or more, 32 g ai/ha or more, 33 g ai/ha or more, 34 g ai/ha or more, 35 g ai/ha or more, 36 g ai/ha or more, 37 g ai/ha or more, 38 g ai/ha or more, 39 g ai/ha or more, 40 g ai/ha or more, 41 g ai/ha or more, 42 g ai/ha or more, 43 g ai/ha or more, 44 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 250 g ai/ha or more, 260 g ai/ha or more, 280 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2050 g ai/ha or more, 2100 g ai/ha or more, 2150 g ai/ha or more, 2200 g ai/ha or more, 2240 g ai/ha or more, 2250 g ai/ha or more, 2300 g ai/ha or more, 2350 g ai/ha or more, 2400 g ai/ha or more, 2450 g ai/ha or more, 2500 g ai/ha or more, 2600 g ai/ha or more, 2700 g ai/ha or more, 2750 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3900 g ai/ha or more, 3950 g ai/ha or more, 4000 g ai/ha or more, 4100 g ai/ha or more, 4200 g ai/ha or more, 4250 g ai/ha or more, 4250 g ai/ha or more, 4300 g ai/ha or more, or 4350 g ai/ha or more, or 4400 g ai/ha or more; in an amount of 4500 g ai/ha or less, such as 4450 g ai/ha or less, 4400 g ai/ha or less, 4350 g ai/ha or less, 4300 g ai/ha or less, 4250 g ai/ha or less, 4100 g ai/ha or less, 4000 g ai/ha or less, 3950 g ai/ha or less, 3900 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2750 g ai/ha or less, 2700 g ai/ha or less, 2600 g ai/ha or less, 2500 g ai/ha or less, 2450 g ai/ha or less, 2400 g ai/ha or less, 2350 g ai/ha or less, 2300 g ai/ha or less, 2250 g ai/ha or less, 2240 g ai/ha or less, 2200 g ai/ha or less, 2150 g ai/ha or less, 2100 g ai/ha or less, 2050 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1240 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 380 g ai/ha or less, 360 g ai/ha or less, 350 g ai/ha or less, 340 g ai/ha or less, 320 g ai/ha or less, 300 g ai/ha or less, 280 g ai/ha or less, 260 g ai/ha or less, 250 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 44 g ai/ha or less, 43 g ai/ha or less, 42 g ai/ha or less, 41 g ai/ha or less, 40 g ai/ha or less, 39 g ai/ha or less, 38 g ai/ha or less, 37 g ai/ha or less, 36 g ai/ha or less, 35 g ai/ha or less, 34 g ai/ha or less, 33 g ai/ha or less, 32 g ai/ha or less, 31 g ai/ha or less, 30 g ai/ha or less, 29 g ai/ha or less, 28 g ai/ha or less, 27 g ai/ha or less, 26 g ai/ha or less, 25 g ai/ha or less, 24 g ai/ha or less, 23 g ai/ha or less, 22 g ai/ha or less, 21 g ai/ha or less, 20 g ai/ha or less, 19 g ai/ha or less, 18 g ai/ha or less, 17 g ai/ha or less, 16 ai/ha or less, 15 g ai/ha or less, 14 g ai/ha or less, 13 g ai/ha or less, 12 g ai/ha or less, 11 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3.5 g ai/ha or less, 3 g ai/ha or less, 2.5 g ai/ha or less, 2 g ai/ha or less, 1.75 g ai/ha or less, 1.5 g ai/ha or less, 1.25 g ai/ha or less, or 1.1 g ai/ha or less; or in an amount ranging from any of the minimum values described above to any of the maximum values described above, such as 1-4500 g ai/ha, 4-3900 g ai/ha, 1.75-2500 g ai/ha, 75-3100 g ai/ha, 90-900 g ai/ha, 55-4200 g ai/ha, 50-2350 g ai/ha, 80-2900 g ai/ha, 120-4100 g ai/ha, 65-2700 g ai/ha, 300-4000 g ai/ha, 1200-3600 g ai/ha, 250-2000 g ai/ha, 1.75-250 g ai/ha, 700-4250 g ai/ha, 39-1100 g ai/ha, or 1.1-4450 g ai/ha.

In some aspects, the herbicidal composition contains a herbicidally effective amount of compounds according to Formula I, and (b) benzobicyclon, benzofenap, bicyclopyrone, fenquinotrione, isoxachlortole, isoxaflutole, lancotrione, mesotrione, pyrasulfotole, pyrazolynate, pyrazoxyfen, sulcotrione, tefuryltrione, tembotrione, tolpyralate, topramezone, agriculturally acceptable salts or esters, or combinations thereof.

### Benzobicyclon

In some aspects, the HPPD inhibitor herbicide can comprise benzobicyclon or an agriculturally acceptable salt or ester thereof. Benzobicyclon, shown below, is 3-[2-chloro-4-(methylsulfonyl)benzoyl]-4-(phenylthio) bicyclo[3.2.1]oct-3-en-2-one. Its herbicidal activity is exemplified in The Pesticide Manual, Seventeenth Edition, 2016. Exemplary uses of benzobicyclon include its use for pre- to early post-emergence control of annual and perennial paddy weeds in direct-seeded and transplanted rice.

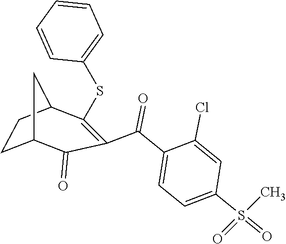 [SMILES: CS(=O)(=O)c1ccc(C(=O)C2=C(Sc3ccccc3)C3CCC(C3)C2=O)c(Cl)c1]

Benzobicyclon can be applied to vegetation or an area adjacent to the vegetation or applied to soil or water to prevent the emergence or growth of vegetation in an amount sufficient to induce a herbicidal effect. In some aspects, benzobicyclon is applied to vegetation or an area adjacent to the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, or 400 g ai/ha or more; in an amount of 401 g ai/ha or less, such as 400 g ai/ha or less, 380 g ai/ha or less, 360 g ai/ha or less, 340 g ai/ha or less, 320 g ai/ha or less, 300 g ai/ha or less, 280 g ai/ha or less, 260 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, or 50 g ai/ha or less; or in an amount ranging from any of the minimum values described above to any of the maximum values described above, such as 50-401 g ai/ha, 75-360 g ai/ha, 100-240 g ai/ha, 150-300 g ai/ha, 170-280 g ai/ha, 140-360 g ai/ha, 200-320 g ai/ha, 80-260 g ai/ha, or 55-400 g ai/ha.

### Benzofenap

In some aspects, the HPPD inhibitor herbicide can comprise benzofenap or an agriculturally acceptable salt or ester thereof. Benzofenap, shown below, is 2-[[4-(2,4-dichloro-3-methylbenzoyl)-1,3-dimethyl-1H-pyrazol-5-yl]oxy]-1-(4-methylphenyl) ethanone. Its herbicidal activity is exemplified in The Pesticide Manual, Seventeenth Edition, 2016. Exemplary uses of benzofenap include its use for control of broadleaf weeds in rice.

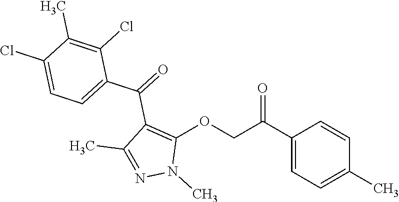 [SMILES: Cc1ccc(C(=O)COc2c(C(=O)c3ccc(Cl)c(C)c3Cl)c(C)nn2C)cc1]

Benzofenap can be applied to vegetation or an area adjacent the vegetation or applied to soil or water to prevent the emergence or growth of vegetation in an amount sufficient to induce a herbicidal effect. In some aspects, benzofenap is applied to vegetation or an area adjacent the vegetation or applied to soil or water to prevent the emergence or growth of vegetation in an amount of 300 g ai/ha or more, such as 310 g ai/ha or more, 320 g ai/ha or more, 340 g ai/ha or more, 360 g ai/ha or more, 380 g ai/ha or more, 400 g ai/ha or more, 450 g ai/ha or more, 500 g ai/ha or more, 550 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, or 1000 g ai/ha or more; in an amount of 1001 g ai/ha or less, such as 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 475 g ai/ha or less, 450 g ai/ha or less, 425 g ai/ha or less, 400 g ai/ha or less, 380 g ai/ha or less, 360 g ai/ha or less, 340 g ai/ha or less, 320 g ai/ha or less, or 310 g ai/ha or less; or in an amount ranging from any of the minimum values described above to any of the maximum values described above, such as 300-1001 g ai/ha, 450-1000 g ai/ha, 380-825 g ai/ha, 400-900 g ai/ha, 450-925 g ai/ha, 550-775 g ai/ha, 600-850 g ai/ha, 475-950 g ai/ha, 340-925 g ai/ha, 650-975 g ai/ha, 725-1000 g ai/ha, 450-850 g ai/ha, 800-950 g ai/ha, or 310-975 g ai/ha.

### Bicyclopyrone

In some aspects, the HPPD inhibitor herbicide can comprise bicyclopyrone or an agriculturally acceptable salt or ester thereof. Bicyclopyrone, shown below, is 4-hydroxy-3-[[2-[(2-methoxyethoxy)methyl]-6-(trifluoromethyl)-3-pyridinyl]carbonyl] bicyclo[3.2.1]oct-3-en-2-one. Its herbicidal activity is exemplified in The Pesticide Manual, Seventeenth Edition, 2016. Exemplary uses of bicyclopyrone include its use for pre- and early post-emergence control of broadleaf and grass weeds in maize and cereals.

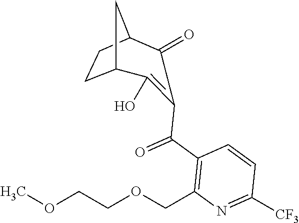 [SMILES: COCCOCc1nc(C(F)(F)F)ccc1C(=O)C1=C(O)C2CCC(C2)C1=O]

Bicyclopyrone can be applied to vegetation or an area adjacent the vegetation or applied to soil or water to prevent the emergence or growth of vegetation in an amount sufficient to induce a herbicidal effect. In some aspects, bicyclopyrone is applied to vegetation or an area adjacent the vegetation or applied to soil or water to prevent the emergence or growth of vegetation in an amount of 20 g ai/ha or more, such as 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, or 400 g ai/ha; in an amount of 401 g ai/ha or less, such as 400 g ai/ha or less, 380 g ai/ha or less, 360 g ai/ha or less, 340 g ai/ha or less, 320 g ai/ha or less, 300 g ai/ha or less, 280 g ai/ha or less, 260 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, or 20 g ai/ha or less; or in an amount ranging from any of the minimum values described above to any of the maximum values described above, such as 20-401 g ai/ha, 30-260 g ai/ha, 40-380 g ai/ha, 55-300 g ai/ha, 60-340 g ai/ha, 45-320 g ai/ha, 25-300 g ai/ha, 100-190 g ai/ha, 200-380 g ai/ha, 50-200 g ai/ha, 30-300 g ai/ha, 85-220 g ai/ha, 30-65 g ai/ha, or 25-400 g ai/ha.

### Fenquinotrione

In some aspects, the HPPD inhibitor herbicide can comprise fenquinotrione or an agriculturally acceptable salt or ester thereof. Fenquinotrione, shown below, is 2-[[8-chloro-3,4-dihydro-4-(4-methoxyphenyl)-3-oxo-2-quinoxalinyl]carbonyl]-1,3-cyclohexanedione. According to the online edition of The Pesticide Manual, fenquinotrione is a herbicide under development for use on rice.

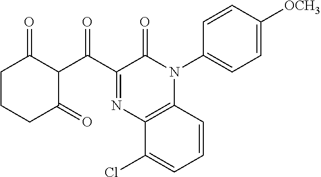 [SMILES: COc1ccc(-n2c(=O)c(C(=O)C3C(=O)CCCC3=O)nc3c(Cl)cccc32)cc1]

### Isoxachlortole

In some aspects, the HPPD inhibitor herbicide can comprise isoxachlortole or an agriculturally acceptable salt or ester thereof. Isoxachlortole, shown below, is [4-chloro-2-(methylsulfonyl)phenyl](5-cyclopropyl-4-isoxazolyl)methanone. According to the online edition of The Pesticide Manual, isoxachlortole is a herbicide that was evaluated by Rhône-Poulenc.

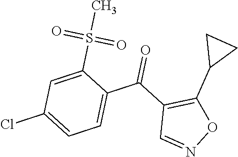 [SMILES: CS(=O)(=O)c1cc(Cl)ccc1C(=O)c1cnoc1C1CC1]

### Isoxaflutole

In some aspects, the HPPD inhibitor herbicide can comprise isoxaflutole or an agriculturally acceptable salt or ester thereof. Isoxaflutole, shown below, is (5-cyclopropyl-4-isoxazolyl)[2-(methylsulfonyl)-4-(trifluoromethyl)phenyl]methanone. Its herbicidal activity is exemplified in The Pesticide Manual. Exemplary uses of isoxaflutole include its use for pre-emergence or pre-plant control of grass and broadleaf weed in maize and sugar cane.

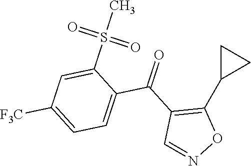 [SMILES: CS(=O)(=O)c1cc(C(F)(F)F)ccc1C(=O)c1cnoc1C1CC1]

Isoxaflutole can be applied to vegetation or an area adjacent the vegetation or applied to soil or water to prevent the emergence or growth of vegetation in an amount sufficient to induce a herbicidal effect. In some aspects, isoxaflutole is applied to vegetation or an area adjacent the vegetation or applied to soil or water to prevent the emergence or growth of vegetation in an amount of 20 g ai/ha or more, such as 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, or 300 g ai/ha or more; in an amount of 301 g ai/ha or less, such as 300 g ai/ha or less, 280 g ai/ha or less, 260 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 25 g ai/ha or less; or in an amount ranging from any of the minimum values described above to any of the maximum values described above, such as 20-301 g ai/ha, 30-300 g ai/ha, 40-170 g ai/ha, 25-260 g ai/ha, 60-120 g ai/ha, 70-190 g ai/ha, 75-240 g ai/ha, 35-200 g ai/ha, 65-220 g ai/ha, 85-140 g ai/ha, 100-300 g ai/ha, 150-280 g ai/ha, 200-301 g ai/ha, 130-200 g ai/ha, 25-100 g ai/ha, 70-90 g ai/ha, or 25-300 g ai/ha.

### Lancotrione

In some aspects, the HPPD inhibitor herbicide can comprise lancotrione or an agriculturally acceptable salt or ester thereof. Lancotrione, shown below, is 2-[2-chloro-3-[2-(1,3-dioxolan-2-yl) ethoxy]-4-(methylsulfonyl)benzoyl]-3-hydroxy-2-cyclohexen-1-one. According to the online edition of The Pesticide Manual, lancotrione is a herbicide under development by Ishihara Sangyo Kaisha, Ltd.

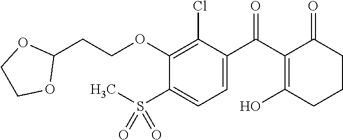 [SMILES: CS(=O)(=O)c1ccc(C(=O)C2=C(O)CCCC2=O)c(Cl)c1OCCC1OCCO1]

### Mesotrione

In some aspects, the HPPD inhibitor herbicide can comprise mesotrione or an agriculturally acceptable salt or ester thereof. Mesotrione, shown below, is 2-[4-(methylsulfonyl)-2-nitrobenzoyl]-1,3-cyclohexanedione. Its herbicidal activity is exemplified in The Pesticide Manual. Exemplary uses of mesotrione include its use for pre- and post-emergence control of broadleaf weeds and some grass weeds in maize.

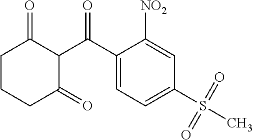 [SMILES: CS(=O)(=O)c1ccc(C(=O)C2C(=O)CCCC2=O)c([N+](=O)[O-])c1]

Mesotrione can be applied to vegetation or an area adjacent the vegetation or applied to soil or water to prevent the emergence or growth of vegetation in an amount sufficient to induce a herbicidal effect. In some aspects, mesotrione is applied to vegetation or an area adjacent the vegetation or applied to soil or water to prevent the emergence or growth of vegetation in an amount of 20 g ai/ha or more, such as 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 220 g ai/ha or more, 225 g ai/ha or more, 240 g ai/ha or more, 250 g ai/ha or more, 260 g ai/ha or more, 275 g ai/ha or more, 280 g ai/ha or more, or 300 g ai/ha or more; in an amount of 301 g ai/ha or less, such as 300 g ai/ha or less, 280 g ai/ha or less, 275 g ai/ha or less, 260 g ai/ha or less, 250 g ai/ha or less, 240 g ai/ha or less, 225 g ai/ha or less, 220 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 125 g ai/ha or less, 130 g ai/ha or less, 120 g ai/ha or less, 110 g ai/ha or less, 100 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 25 g ai/ha or less; or in an amount ranging from any of the minimum values described above to any of the maximum values described above, such as 20-301 g ai/ha, 30-275 g ai/ha, 40-140 g ai/ha, 50-250 g ai/ha, 60-301 g ai/ha, 25-280 g ai/ha, 60-125 g ai/ha, 70-250 g ai/ha, 90-225 g ai/ha, 110-220 g ai/ha, 40-175 g ai/ha, 65-160 g ai/ha, 70-200 g ai/ha, 80-180 g ai/ha, 90-250 g ai/ha, 140-225 g ai/ha, 110-240 g ai/ha, 120-175 g ai/ha, 110-150 g ai/ha, 25-85 g ai/ha, or 25-300 g ai/ha.

### Pyrasulfotole

In some aspects, the HPPD inhibitor herbicide can comprise pyrasulfotole or an agriculturally acceptable salt or ester thereof. Pyrasulfotole, shown below, is (5-hydroxy-1,3-dimethyl-1H-pyrazol-4-yl)[2-(methylsulfonyl)-4-(trifluoromethyl)phenyl]methanone. Its herbicidal activity is exemplified in The Pesticide Manual. Exemplary uses of pyrasulfotole include its use in mixture with mefenpyr-diethyl and bromoxynil or MCPA ester, for post-emergence control of broadleaf weeds in cereals.

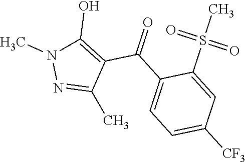 [SMILES: Cc1nn(C)c(O)c1C(=O)c1ccc(C(F)(F)F)cc1S(C)(=O)=O]

Pyrasulfotole can be applied to vegetation or an area adjacent the vegetation or applied to soil or water to prevent the emergence or growth of vegetation in an amount sufficient to induce a herbicidal effect. In some aspects, pyrasulfotole is applied to vegetation or an area adjacent the vegetation or applied to soil or water to prevent the emergence or growth of vegetation in an amount of 1 g ai/ha or more, such as 1.1 g ai/ha or more, 1.25 g ai/ha or more, 1.4 g ai/ha or more, 1.5 g ai/ha or more, 1.6 g ai/ha or more, 1.75 g ai/ha or more, 1.9 g ai/ha or more, 2 g ai/ha or more, 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.5 g ai/ha or more, 5 g ai/ha or more, 6 g ai/ha or more, 7 g ai/ha or more, 8 g ai/ha or more, 9 g ai/ha or more, 9.5 g ai/ha or more, 10 g ai/ha or more, 10.5 g ai/ha or more, 11 g ai/ha or more, 11.5 g ai/ha or more, 12 g ai/ha or more, 12.5 g ai/ha or more, 13 g ai/ha or more, 13.5 g ai/ha or more, 14 g ai/ha or more, 14.5 g ai/ha or more, 15 g ai/ha or more, 15.5 g ai/ha or more, 16 g ai/ha or more, 16.5 g ai/ha or more, 17 g ai/ha or more, 17.5 g ai/ha or more, 18 g ai/ha or more, 18.5 g ai/ha or more, 20 g ai/ha or more, 21 g ai/ha or more, 22 g ai/ha or more, 23 g ai/ha or more, 24 g ai/ha or more, 25 g ai/ha or more, 26 g ai/ha or more, 27 g ai/ha or more, 28 g ai/ha or more, 29 g ai/ha or more, 30 g ai/ha or more, 31 g ai/ha or more, 32 g ai/ha or more, 33 g ai/ha or more, 34 g ai/ha or more, 35 g ai/ha or more, 36 g ai/ha or more, 37 g ai/ha or more, 38 g ai/ha or more, 39 g ai/ha or more, 40 g ai/ha or more, 41 g ai/ha or more, 42 g ai/ha or more, 43 g ai/ha or more, 44 g ai/ha or more, 45 g ai/ha or more, or 47 g ai/ha or more; in an amount of 50 g ai/ha or less, such as 47 g ai/ha or less, 45 g ai/ha or less, 44 g ai/ha or less, 43 g ai/ha or less, 42 g ai/ha or less, 41 g ai/ha or less, 40 g ai/ha or less, 39 g ai/ha or less, 38 g ai/ha or less, 37 g ai/ha or less, 36 g ai/ha or less, 35 g ai/ha or less, 34 g ai/ha or less, 33 g ai/ha or less, 32 g ai/ha or less, 31 g ai/ha or less, 30 g ai/ha or less, 29 g ai/ha or less, 28 g ai/ha or less, 27 g ai/ha or less, 26 g ai/ha or less, 25 g ai/ha or less, 24 g ai/ha or less, 23 g ai/ha or less, 22 g ai/ha or less, 21 g ai/ha or less, 20 g ai/ha or less, 19 g ai/ha or less, 18.5 g ai/ha or less, 18 g ai/ha or less, 17.5 g ai/ha or less, 17 g ai/ha or less, 16.5 g ai/ha or less, 16 g ai/ha or less, 15.5 g ai/ha or less, 15 g ai/ha or less, 14.5 g ai/ha or less, 14 g ai/ha or less, 13.5 g ai/ha or less, 13 g ai/ha or less, 12.5 g ai/ha or less, 12 g ai/ha or less, 11.5 g ai/ha or less, 11 g ai/ha or less, 10.5 g ai/ha or less, 10 g ai/ha or less, 9.5 g ai/ha or less, 9 g ai/ha or less, 8.5 g ai/ha or less, 8 g ai/ha or less, 7.5 g ai/ha or less, 7 g ai/ha or less, 6.5 g ai/ha or less, 6 g ai/ha or less, 5.5 g ai/ha or less, 5 g ai/ha or less, 4.5 g ai/ha or less, 4 g ai/ha or less, 3.75 g ai/ha or less, 3.5 g ai/ha or less, 3.25 g ai/ha or less, 3 g ai/ha or less, 2.75 g ai/ha or less, 2.5 g ai/ha or less, 2.25 g ai/ha or less, 2 g ai/ha or less, 1.9 g ai/ha or less, 1.75 g ai/ha or less, 1.6 g ai/ha or less, 1.5 g ai/ha or less, 1.4 g ai/ha or less, 1.25 g ai/ha or less, or 1.1 g ai/ha or less; or in an amount ranging from any of the minimum values described above to any of the maximum values described above, such as 1-50 g ai/ha, 1.4-45 g ai/ha, 1.2-42 g ai/ha, 2.5-35 g ai/ha, 4-30 g ai/ha, 6.5-29 g ai/ha, 1.25-24 g ai/ha, 3.75-18.5 g ai/ha, 2.5-38 g ai/ha, 11-17 g ai/ha, 8-16 g ai/ha, 2.75-19 g ai/ha, 3-25 g ai/ha, 7-23 g ai/ha, 3.5-16 g ai/ha, 10-41 g ai/ha,

### Pyrazolynate

In some aspects, the HPPD inhibitor herbicide can comprise pyrazolynate or an agriculturally acceptable salt or ester thereof. Pyrazolynate, shown below, is (2,4-dichlorophenyl)[1,3-dimethyl-5-[[(4-methylphenyl)sulfonyl]oxy]-1H-pyrazol-4-yl]methanone. Its herbicidal activity is exemplified in The Pesticide Manual. Exemplary uses of pyrazolynate include its use to control grasses, sedges, Potamogeton distinctus, Sagittaria pygmaea, Sagittaria trifolia and Alisma canaliculatum in paddy rice.

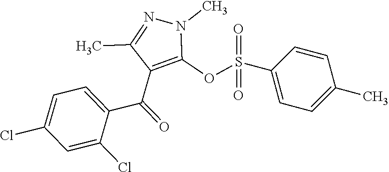 [SMILES: Cc1ccc(S(=O)(=O)Oc2c(C(=O)c3ccc(Cl)cc3Cl)c(C)nn2C)cc1]

Pyrazolynate can be applied to vegetation or an area adjacent the vegetation or applied to soil or water to prevent the emergence or growth of vegetation in an amount sufficient to induce a herbicidal effect. In some aspects, pyrazolynate is applied to vegetation or an area adjacent the vegetation or applied to soil or water to prevent the emergence or growth of vegetation in an amount of 200 g ai/ha or more, such as 225 g ai/ha or more, 250 g ai/ha or more, 275 g ai/ha or more, 300 g ai/ha or more, 350 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1200 g ai/ha or more, 1400 g ai/ha or more, 1600 g ai/ha or more, 1800 g ai/ha or more, 2000 g ai/ha or more, 2200 g ai/ha or more, 2400 g ai/ha or more, 2500 g ai/ha or more, 2600 g ai/ha or more, 2800 g ai/ha or more, 3000 g ai/ha or more, 3200 g ai/ha or more, 3400 g ai/ha or more, 3500 g ai/ha or more, 3600 g ai/ha or more, 3800 g ai/ha or more, 4000 g ai/ha or more, 4200 g ai/ha or more, or 4400 g ai/ha or more; in an amount of 4500 g ai/ha or less, such as 4400 g ai/ha or less, 4200 g ai/ha or less, 4000 g ai/ha or less, 3800 g ai/ha or less, 3600 g ai/ha or less, 3500 g ai/ha or less, 3400 g ai/ha or less, 3200 g ai/ha or less, 3000 g ai/ha or less, 2800 g ai/ha or less, 2600 g ai/ha or less, 2500 g ai/ha or less, 2400 g ai/ha or less, 2200 g ai/ha or less, 2000 g ai/ha or less, 1800 g ai/ha or less, 1600 g ai/ha or less, 1400 g ai/ha or less, 1200 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 350 g ai/ha or less, 300 g ai/ha or less, 275 g ai/ha or less, 250 g ai/ha or less, or 225 g ai/ha or less; or in an amount ranging from any of the minimum values described above to any of the maximum values described above, such as 200-4500 g ai/ha, 2000-4000 g ai/ha, 400-1600 g ai/ha, 1600-3800 g ai/ha, 750-3000 g ai/ha, 500-2200 g ai/ha, 1600-2500 g ai/ha, 250-800 g ai/ha, 2500-3400 g ai/ha, 1200-3500 g ai/ha, 350-1000 g ai/ha, or 225-4400 g ai/ha.

### Pyrazoxyfen

In some aspects, the HPPD inhibitor herbicide can comprise pyrazoxyfen or an agriculturally acceptable salt or ester thereof. Pyrazoxyfen, shown below, is 2-[[4-(2,4-dichlorobenzoyl)-1,3-dimethyl-1H-pyrazol-5-yl]oxy]-1-phenylethanone. Its herbicidal activity is exemplified in The Pesticide Manual. Exemplary uses of pyrazoxyfen include pre- or post-emergence control of annual and perennial weeds in transplanted or direct-seeded paddy rice.

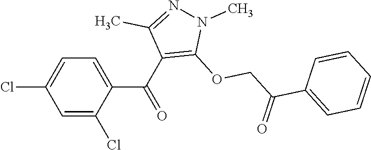 [SMILES: Cc1nn(C)c(OCC(=O)c2ccccc2)c1C(=O)c1ccc(Cl)cc1Cl]

Pyrazoxyfen can be applied to vegetation or an area adjacent the vegetation or applied to soil or water to prevent the emergence or growth of vegetation in an amount sufficient to induce a herbicidal effect. In some aspects, pyrazoxyfen is applied to vegetation or an area adjacent the vegetation or applied to soil or water to prevent the emergence or growth of vegetation in an amount of 1000 g ai/ha or more, such as 1100 g ai/ha or more, 1200 g ai/ha or more, 1400 g ai/ha or more, 1600 g ai/ha or more, 1800 g ai/ha or more, 2000 g ai/ha or more, 2200 g ai/ha or more, 2400 g ai/ha or more, 2500 g ai/ha or more, 2600 g ai/ha or more, 2800 g ai/ha or more, 3000 g ai/ha or more, 3200 g ai/ha or more, 3400 g ai/ha or more, 3500 g ai/ha or more, 3600 g ai/ha or more, 3800 g ai/ha or more, 4000 g ai/ha or more, 4200 g ai/ha or more, or 4400 g ai/ha or more; in an amount of 4500 g ai/ha or less, such as 4400 g ai/ha or less, 4200 g ai/ha or less, 4000 g ai/ha or less, 3800 g ai/ha or less, 3600 g ai/ha or less, 3500 g ai/ha or less, 3400 g ai/ha or less, 3200 g ai/ha or less, 3000 g ai/ha or less, 2800 g ai/ha or less, 2600 g ai/ha or less, 2500 g ai/ha or less, 2400 g ai/ha or less, 2200 g ai/ha or less, 2000 g ai/ha or less, 1800 g ai/ha or less, 1600 g ai/ha or less, 1400 g ai/ha or less, 1200 g ai/ha or less, or 1100 g ai/ha or less; or in an amount ranging from any of the minimum values described above to any of the maximum values described above, such as 1000-4500 g ai/ha, 1100-4000 g ai/ha, 1400-3600 g ai/ha, 1200-1800 g ai/ha, 1800-4000 g ai/ha, 2200-4500 g ai/ha, 1500-3200 g ai/ha, 2000-3500 g ai/ha, 2500-3400 g ai/ha, or 1100-4400 g ai/ha.

### Sulcotrione

In some aspects, the HPPD inhibitor herbicide can comprise sulcotrione or an agriculturally acceptable salt or ester thereof. Sulcotrione, shown below, is 2-[2-chloro-4-(methylsulfonyl)benzoyl]-1,3-cyclohexanedione. Its herbicidal activity is exemplified in The Pesticide Manual. Exemplary uses of sulcotrione include its use to control broadleaf weeds and grasses post-emergence in maize and sugar cane.

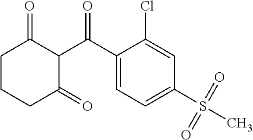 [SMILES: CS(=O)(=O)c1ccc(C(=O)C2C(=O)CCCC2=O)c(Cl)c1]

Sulcotrione can be applied to vegetation or an area adjacent the vegetation or applied to soil or water to prevent the emergence or growth of vegetation in an amount sufficient to induce a herbicidal effect. In some aspects, sulcotrione is applied to vegetation or an area adjacent the vegetation or applied to soil or water to prevent the emergence or growth of vegetation in an amount of 20 g ai/ha or more, such as 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, or 580 g ai/ha or more; in an amount of 600 g ai/ha or less, such as 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 280 g ai/ha or less, 260 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 25 g ai/ha or less; or in an amount ranging from any of the minimum values described above to any of the maximum values described above, such as 20-600 g ai/ha, 40-400 g ai/ha, 65-420 g ai/ha, 120-240 g ai/ha, 300-460 g ai/ha, 400-580 g ai/ha, 30-500 g ai/ha, 250-560 g ai/ha, 250-440 g ai/ha, 75-520 g ai/ha, 35-300 g ai/ha, 90-540 g ai/ha, 150-400 g ai/ha, 400-560 g ai/ha, 50-300 g ai/ha, 110-480 g ai/ha, 75-420 g ai/ha, 65-360 g ai/ha, 170-300 g ai/ha, 200-300 g ai/ha, 220-600 g ai/ha, or 25-580 g ai/ha.

### Tefuryltrione

In some aspects, the HPPD inhibitor herbicide can comprise tefuryltrione or an agriculturally acceptable salt or ester thereof. Tefuryltrione, shown below, is 2-[2-chloro-4-(methylsulfonyl)-3-[[(tetrahydro-2-furanyl)-methoxy]-methyl]-benzoyl]-1,3-cyclohexandione. According to the online edition of The Pesticide Manual, tefuryltrione is a herbicide jointly developed by Bayer CropScience, Hokko Chemical, and Zen-Noh for pre- and post-emergence control of annual and perennial broadleaf weeds in rice and cereals.

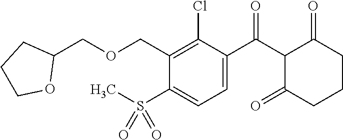 [SMILES: CS(=O)(=O)c1ccc(C(=O)C2C(=O)CCCC2=O)c(Cl)c1COCC1CCCO1]

### Tembotrione

In some aspects, the HPPD inhibitor herbicide can comprise tembotrione or an agriculturally acceptable salt or ester thereof. Tembotrione, shown below, is 2-[2-chloro-4-(methylsulfonyl)-3-[(2,2,2-trifluoroethoxy)methyl]benzoyl]-1,3-cyclohexanedione. Its herbicidal activity is exemplified in The Pesticide Manual. Exemplary uses of tembotrione include its use for post-emergence control of grass and broadleaf weed species in maize.

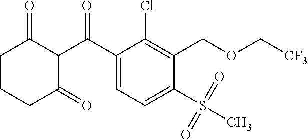 [SMILES: CS(=O)(=O)c1ccc(C(=O)C2C(=O)CCCC2=O)c(Cl)c1COCC(F)(F)F]

Tembotrione can be applied to vegetation or an area adjacent the vegetation or applied to soil or water to prevent the emergence or growth of vegetation in an amount sufficient to induce a herbicidal effect. In some aspects, tembotrione is applied to vegetation or an area adjacent the vegetation or applied to soil or water to prevent the emergence or growth of vegetation in an amount of 1 g ai/ha or more, such as 1.1 g ai/ha or more, 1.25 g ai/ha or more, 1.4 g ai/ha or more, 1.5 g ai/ha or more, 1.6 g ai/ha or more, 1.75 g ai/ha or more, 1.9 g ai/ha or more, 2 g ai/ha or more, 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.5 g ai/ha or more, 5 g ai/ha or more, 6 g ai/ha or more, 7 g ai/ha or more, 8 g ai/ha or more, 9 g ai/ha or more, 9.5 g ai/ha or more, 10 g ai/ha or more, 10.5 g ai/ha or more, 11 g ai/ha or more, 11.5 g ai/ha or more, 12 g ai/ha or more, 12.5 g ai/ha or more, 13 g ai/ha or more, 13.5 g ai/ha or more, 14 g ai/ha or more, 14.5 g ai/ha or more, 15 g ai/ha or more, 15.5 g ai/ha or more, 16 g ai/ha or more, 16.5 g ai/ha or more, 17 g ai/ha or more, 17.5 g ai/ha or more, 18 g ai/ha or more, 18.5 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, or 200 g ai/ha or more; in an amount of 200 g ai/ha or less, such as 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 19 g ai/ha or less, 18.5 g ai/ha or less, 18 g ai/ha or less, 17.5 g ai/ha or less, 17 g ai/ha or less, 16.5 g ai/ha or less, 16 g ai/ha or less, 15.5 g ai/ha or less, 15 g ai/ha or less, 14.5 g ai/ha or less, 14 g ai/ha or less, 13.5 g ai/ha or less, 13 g ai/ha or less, 12.5 g ai/ha or less, 12 g ai/ha or less, 11.5 g ai/ha or less, 11 g ai/ha or less, 10.5 g ai/ha or less, 10 g ai/ha or less, 9.5 g ai/ha or less, 9 g ai/ha or less, 8.5 g ai/ha or less, 8 g ai/ha or less, 7.5 g ai/ha or less, 7 g ai/ha or less, 6.5 g ai/ha or less, 6 g ai/ha or less, 5.5 g ai/ha or less, 5 g ai/ha or less, 4.5 g ai/ha or less, 4 g ai/ha or less, 3.75 g ai/ha or less, 3.5 g ai/ha or less, 3.25 g ai/ha or less, 3 g ai/ha or less, 2.75 g ai/ha or less, 2.5 g ai/ha or less, 2.25 g ai/ha or less, 2 g ai/ha or less, 1.9 g ai/ha or less, 1.75 g ai/ha or less, 1.6 g ai/ha or less, 1.5 g ai/ha or less, 1.4 g ai/ha or less, 1.25 g ai/ha or less, or 1.1 g ai/ha or less; or in an amount ranging from any of the minimum values described above to any of the maximum values described above, such as 1-200 g ai/ha, 1.4-75 g ai/ha, 3.5-150 g ai/ha, 10-140 g ai/ha, 6-75 g ai/ha, 8-180 g ai/ha, 1.5-25 g ai/ha, 2.5-15 g ai/ha, 2.25-10 g ai/ha, 25-120 g ai/ha, 1.9-130 g ai/ha, 20-40 g ai/ha, 30-180 g ai/ha, 45-190 g ai/ha, 3.5-70 g ai/ha, 6-130 g ai/ha, 70-90 g ai/ha, 15-110 g ai/ha, 120-140 g ai/ha, or 1.1-190 g ai/ha.

### Tolpyralate

In some aspects, the HPPD inhibitor herbicide can comprise tolpyralate or an agriculturally acceptable salt or ester thereof. Tolpyralate, shown below, is 1-[[1-ethyl-4-[3-(2-methoxyethoxy)-2-methyl-4-(methylsulfonyl)benzoyl]-1H-pyrazol-5-yl]oxy]ethyl methyl carbonate. According to the online edition of The Pesticide Manual, tefuryltrione is a herbicide developed by Ishihara Sangyo Kaisha Ltd. for pre- and post-emergence control of broadleaf and grass weeds, including Palmer amaranth, water hemp, giant ragweed, and giant foxtail in maize.

### Topramezone

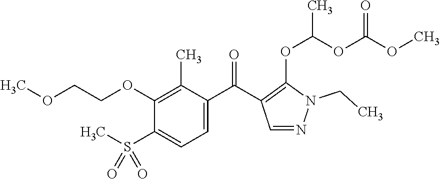

In some aspects, the HPPD inhibitor herbicide can comprise topramezone or an agriculturally acceptable salt or ester thereof. Topramezone, shown below, is [3-(4,5-dihydro-3-isoxazolyl)-2-methyl-4-(methylsulfonyl)phenyl](5-hydroxy-1-methyl-1H-pyrazol-4-yl) methanone. Its herbicidal activity is exemplified in The Pesticide Manual. Exemplary uses of topramezone include its use for post-emergence weed control in maize.

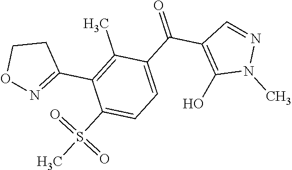 [SMILES: Cc1c(C(=O)c2cnn(C)c2O)ccc(S(C)(=O)=O)c1C1=NOCC1]

Topramezone can be applied to vegetation or an area adjacent the vegetation or applied to soil or water to prevent the emergence or growth of vegetation in an amount sufficient to induce a herbicidal effect. In some aspects, topramezone is applied to vegetation or an area adjacent the vegetation or applied to soil or water to prevent the emergence or growth of vegetation in an amount of 1 g ai/ha or more, such as 1.1 g ai/ha or more, 1.25 g ai/ha or more, 1.4 g ai/ha or more, 1.5 g ai/ha or more, 1.6 g ai/ha or more, 1.75 g ai/ha or more, 1.9 g ai/ha or more, 2 g ai/ha or more, 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.5 g ai/ha or more, 5 g ai/ha or more, 6 g ai/ha or more, 7 g ai/ha or more, 8 g ai/ha or more, 9 g ai/ha or more, 9.5 g ai/ha or more, 10 g ai/ha or more, 10.5 g ai/ha or more, 11 g ai/ha or more, 11.5 g ai/ha or more, 12 g ai/ha or more, 12.5 g ai/ha or more, 13 g ai/ha or more, 13.5 g ai/ha or more, 14 g ai/ha or more, 14.5 g ai/ha or more, 15 g ai/ha or more, 15.5 g ai/ha or more, 16 g ai/ha or more, 16.5 g ai/ha or more, 17 g ai/ha or more, 17.5 g ai/ha or more, 18 g ai/ha or more, 18.5 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, or 100 g ai/ha or more; in an amount of 100 g ai/ha or less, such as 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 19 g ai/ha or less, 18.5 g ai/ha or less, 18 g ai/ha or less, 17.5 g ai/ha or less, 17 g ai/ha or less, 16.5 g ai/ha or less, 16 g ai/ha or less, 15.5 g ai/ha or less, 15 g ai/ha or less, 14.5 g ai/ha or less, 14 g ai/ha or less, 13.5 g ai/ha or less, 13 g ai/ha or less, 12.5 g ai/ha or less, 12 g ai/ha or less, 11.5 g ai/ha or less, 11 g ai/ha or less, 10.5 g ai/ha or less, 10 g ai/ha or less, 9.5 g ai/ha or less, 9 g ai/ha or less, 8.5 g ai/ha or less, 8 g ai/ha or less, 7.5 g ai/ha or less, 7 g ai/ha or less, 6.5 g ai/ha or less, 6 g ai/ha or less, 5.5 g ai/ha or less, 5 g ai/ha or less, 4.5 g ai/ha or less, 4 g ai/ha or less, 3.75 g ai/ha or less, 3.5 g ai/ha or less, 3.25 g ai/ha or less, 3 g ai/ha or less, 2.75 g ai/ha or less, 2.5 g ai/ha or less, 2.25 g ai/ha or less, 2 g ai/ha or less, 1.9 g ai/ha or less, 1.75 g ai/ha or less, 1.6 g ai/ha or less, 1.5 g ai/ha or less, 1.4 g ai/ha or less, 1.25 g ai/ha or less, or 1.1 g ai/ha or less; or in an amount ranging from any of the minimum values described above to any of the maximum values described above, such as 1-100 g ai/ha, 2-90 g ai/ha, 4-80 g ai/ha, 2.5-75 g ai/ha, 9-60 g ai/ha, 10-55 g ai/ha, 3-25 g ai/ha, 8-15 g ai/ha, 1.5-10 g ai/ha, 5-80 g ai/ha, 6.5-45 g ai/ha, 15-85 g ai/ha, 20-90 g ai/ha, 2.5-40 g ai/ha, 3-30 g ai/ha, 9-85 g ai/ha, or 1.5-95 g ai/ha.

### ACCase Inhibitor Herbicides

In addition to the compounds according to Formula I or agriculturally acceptable N-oxide, salt or ester thereof, the compositions include an acetyl CoA carboxylase (ACCase) inhibitor herbicide or an agriculturally acceptable salt or ester thereof. ACCase inhibitor herbicides inhibit lipid biosynthesis in the plant. Examples of ACCase inhibitor herbicides include aryloxyphenoxypropionates, cyclohexanediones, and phenylpyrazolines. In some aspects, the ACCase inhibitor herbicide can include an aryloxyphenoxypropionate herbicide. In some aspects, the ACCase inhibitor herbicide can include a cyclohexanedione herbicide. In some aspects, the ACCase inhibitor herbicide can include a phenylpyrazoline herbicide.

In some aspects, the composition can include an ACCase inhibitor selected from the group of cyclohexanediones, aryloxyphenoxypropionates, phenylpyrazolines, or combinations thereof. In some cases, the composition can include clodinafop, cyhalofop, diclofop, fenoxaprop, fenthiaprop, fluazifop, haloxyfop, metamifop, propaquizafop, quizalofop, agriculturally acceptable salts or esters thereof, or combinations thereof. In some cases, the composition can include, alloxydim, butroxydim, clethodim, cloproxydim, cycloxydim, profoxydim, pyriflubenzoxim, sethoxydim, tepraloxydim, tralkoxydim, agriculturally acceptable salts or esters thereof, or combinations thereof. In some cases, the composition can include pinoxaden

In some aspects, the composition can include an ACCase inhibitor herbicide selected from the group consisting of alloxydim, butroxydim, clethodim, clodinafop, cloproxydim, cycloxydim, cyhalofop, diclofop, fenoxaprop, fenthiaprop, fluazifop, haloxyfop, metamifop, pinoxaden, profoxydim, propaquizafop, quizalofop, sethoxydim, tepraloxydim, tralkoxydim, agriculturally acceptable salts and esters thereof, and combinations thereof.

The ACCase inhibitor herbicide or agriculturally acceptable salt or ester thereof can be applied to vegetation or an area adjacent the vegetation or applied to soil or water to prevent the emergence or growth of vegetation in an amount sufficient to induce a herbicidal effect. In some aspects, the ACCase inhibitor herbicide or agriculturally acceptable salt or ester thereof is applied to vegetation or an area adjacent the vegetation or applied to soil or water to prevent the emergence or growth of vegetation in an amount of 2 g ai/ha or more, such as 2.5 g ai/ha or more, 3 g ai/ha or more, 3.5 g ai/ha or more, 4 g ai/ha or more, 4.5 g ai/ha or more, 5 g ai/ha or more, 6 g ai/ha or more, 7 g ai/ha or more, 8 g ai/ha or more, 9 g ai/ha or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 250 g ai/ha or more, 260 g ai/ha or more, 280 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, or 1450 g ai/ha or more; in an amount of 1500 g ai/ha or less, such as 1450 g ai/ha or less, 1400 g ai/ha or less, 1350 g ai/ha or less, 1300 g ai/ha or less, 1250 g ai/ha or less, 1240 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 380 g ai/ha or less, 360 g ai/ha or less, 350 g ai/ha or less, 340 g ai/ha or less, 320 g ai/ha or less, 300 g ai/ha or less, 280 g ai/ha or less, 260 g ai/ha or less, 250 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9 g ai/ha or less, 8 g ai/ha or less, 7 g ai/ha or less, 6 g ai/ha or less, 5 g ai/ha or less, 4.5 g ai/ha or less, 4 g ai/ha or less, 3.5 g ai/ha or less, 3 g ai/ha or less, or 2.5 g ai/ha or less; or in an amount ranging from any of the minimum values described above to any of the maximum values described above, such as 2-1500 g ai/ha, 5-1300 g ai/ha, 6-250 g ai/ha, 75-1240 g ai/ha, 90-900 g ai/ha, 55-290 g ai/ha, 7-600 g ai/ha, 20-700 g ai/ha, 190-1450 g ai/ha, 65-1100 g ai/ha, 320-1000 g ai/ha, 250-750 g ai/ha, 700-1200 g ai/ha, 850-1400 g ai/ha, or 2.5-1450 g ai/ha.

In some aspects, the composition contains: compounds according to Formula I; and (b) alloxydim, butroxydim, clethodim, clodinafop, cloproxydim, cycloxydim, cyhalofop, diclofop, fenoxaprop, fenthiaprop, fluazifop, haloxyfop, metamifop, pinoxaden, profoxydim, propaquizafop, quizalofop, sethoxydim, tepraloxydim, tralkoxydim, agriculturally acceptable salts and esters thereof, and combinations thereof.

### Alloxydim

Compositions and methods of the present disclosure can include alloxydim or an agriculturally acceptable salt or ester thereof. Alloxydim, shown below, is a cyclohexanedione herbicide that provides post-emergence control of grass weeds and volunteer cereals in sugar beet, vegetables and broad-leaved crops. Alloxydim, as well as methods of preparing alloxydim, are known in the art. Its herbicidal activity is described in The Pesticide Manual, Seventeenth Edition, 2016.

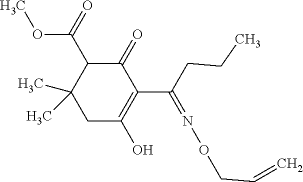 [SMILES: C=CCON=C(CCC)C1=C(O)CC(C)(C)C(C(=O)OC)C1=O]

Alloxydim can be applied to vegetation or an area adjacent the vegetation or applied to soil or water to prevent the emergence or growth of vegetation in an amount sufficient to induce a herbicidal effect. In some aspects, alloxydim is applied to vegetation or an area adjacent the vegetation or applied to soil or water to prevent the emergence or growth of vegetation in an amount of 299 g ai/ha or more, such as 300 g ai/ha or more, 320 g ai/ha or more, 340 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 620 g ai/ha or more, 640 g ai/ha or more, 660 g ai/ha or more, 680 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1200 g ai/ha or more, 1300 g ai/ha or more, 1400 g ai/ha or more, or 1500 g ai/ha or more; in an amount of 1501 g ai/ha or less, such as 1500 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 680 g ai/ha or less, 660 g ai/ha or less, 640 g ai/ha or less, 620 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, or 300 g ai/ha or less; or from any of the minimum values described above to any of the maximum values described above, such as 299-1501 g ai/ha, 380-1250 g ai/ha, 440-850 g ai/ha, 700-1000 g ai/ha, 320-950 g ai/ha, 680-1350 g ai/ha, 400-1050 g ai/ha, 500-1000 g ai/ha, 600-900 g ai/ha, 700-1300 g ai/ha, 500-700 g ai/ha, or 320-1400 g ai/ha.

### Butroxydim

Compositions and methods of the present disclosure can include butroxydim or an agriculturally acceptable salt or ester thereof. Butroxydim, shown below, is a cyclohexanedione herbicide that provides post-emergence control of annual grasses in many broad-leaved crops. Butroxydim, as well as methods of preparing butroxydim, are known in the art. Its herbicidal activity is described in The Pesticide Manual, Seventeenth Edition, 2016.

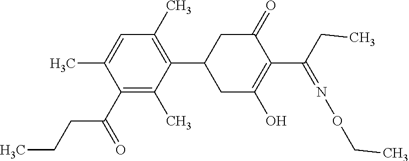 [SMILES: CCCC(=O)c1c(C)cc(C)c(C2CC(=O)C(C(CC)=NOCC)=C(O)C2)c1C]

Butroxydim can be applied to vegetation or an area adjacent the vegetation or applied to soil or water to prevent the emergence or growth of vegetation in an amount sufficient to induce a herbicidal effect. In some aspects, butroxydim is applied to vegetation or an area adjacent the vegetation or applied to soil or water to prevent the emergence or growth of vegetation in an amount of 9 g ai/ha or more, such as 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 105 g ai/ha or more, 110 g ai/ha or more, 115 g ai/ha or more, or 120 g ai/ha or more; in an amount of 121 g ai/ha or less, such as 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, or 10 g ai/ha or less; or in an amount ranging from any of the minimum values described above to any of the maximum values described above, such as 9-121 g ai/ha, 10-115 g ai/ha, 20-110 g ai/ha, 15-85 g ai/ha, 30-100 g ai/ha, 50-95 g ai/ha, 15-60 g ai/ha, 25-85 g ai/ha, 40-80 g ai/ha, 20-75 g ai/ha, 35-110 g ai/ha, 25-105 g ai/ha, 15-55 g ai/ha, or 10-120 g ai/ha.

### Clethodim

Compositions and methods of the present disclosure can include clethodim or an agriculturally acceptable salt or ester thereof. Clethodim, shown below, is a cyclohexanedione herbicide that provides post-emergence control of annual and perennial grasses in a wide range of broad-leaved crops, including such field crops as canola/oilseed rape, soybean, cotton, flax, sunflowers, alfalfa, peanuts, sugar beet, tobacco, and potatoes, as well as vegetable crops, trees, and vines. Clethodim, as well as methods of preparing clethodim, are known in the art. Its herbicidal activity is described in The Pesticide Manual, Seventeenth Edition, 2016.

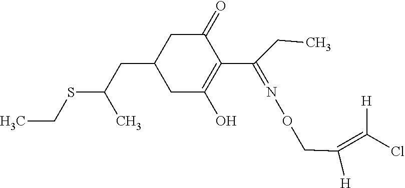 [SMILES: CCSC(C)CC1CC(=O)C(C(CC)=NOC/C=C/Cl)=C(O)C1]

Clethodim can be applied to vegetation or an area adjacent the vegetation or applied to soil or water to prevent the emergence or growth of vegetation in an amount sufficient to induce a herbicidal effect. In some aspects, clethodim is applied to vegetation or an area adjacent the vegetation or applied to soil or water to prevent the emergence or growth of vegetation in an amount of 2 g ai/ha or more, such as 3 g ai/ha or more, 4 g ai/ha or more, 6 g ai/ha or more, 8 g ai/ha or more, 10 g ai/ha or more, 12 g ai/ha or more, 14 g ai/ha or more, 16 g ai/ha or more, 18 g ai/ha or more, 20 g ai/ha or more, 22 g ai/ha or more, 24 g ai/ha or more, 26 g ai/ha or more, 28 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 80 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 620 g ai/ha or more, 640 g ai/ha or more, 660 g ai/ha or more, 680 g ai/ha or more, 700 g ai/ha or more, 750 g ai/ha or more, 800 g ai/ha or more, 850 g ai/ha or more, or 875 g ai/ha or more; in an amount of 900 g ai/ha or less, such as 875 g ai/ha or less, 850 g ai/ha or less, 800 g ai/ha or less, 750 g ai/ha or less, 700 g ai/ha or less, 680 g ai/ha or less, 660 g ai/ha or less, 640 g ai/ha or less, 620 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 235 g ai/ha or less, 230 g ai/ha or less, 225 g ai/ha or less, 220 g ai/ha or less, 215 g ai/ha or less, 210 g ai/ha or less, 205 g ai/ha or less, 200 g ai/ha or less, 195 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 80 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 28 g ai/ha or less, 26 g ai/ha or less, 24 g ai/ha or less, 22 g ai/ha or less, 20 g ai/ha or less, 18 g ai/ha or less, 16 g ai/ha or less, 14 g ai/ha or less, 12 g ai/ha or less, 10 g ai/ha or less, 8 g ai/ha or less, 6 g ai/ha or less, 4 g ai/ha or less, or 3 g ai/ha or less, or in an amount ranging from any of the minimum values described above to any of the maximum values described above, such as 2-900 g ai/ha, 3-800 g ai/ha, 6-700 g ai/ha, 22-500 g ai/ha, 12-600 g ai/ha, 5-500 g ai/ha, 40-440 g ai/ha, 20-700 g ai/ha, 80-400 g ai/ha, 60-270 g ai/ha, 80-750 g ai/ha, 100-290 g ai/ha, 175-750 g ai/ha, 300-900 g ai/ha, 3-100 g ai/ha, 35-320 g ai/ha, 3-205 g ai/ha, 4-50 g ai/ha, or 3-875 g ai/ha.

### Clodinafop

Compositions and methods of the present disclosure can include clodinafop or an agriculturally acceptable salt or ester thereof. Clodinafop-propargyl, shown below, is prop-2-ynyl (R)-2-[4-(5-chloro-3-fluoropyridin-2-yloxy) phenoxy]propionate and is used for post-emergence control of annual grasses, including Avena, Lolium, Setaria, Phalaris, and Alopecurus spp., in cereals.

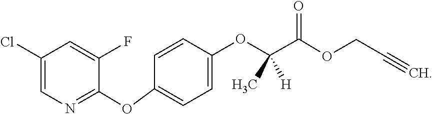 [SMILES: C#CCOC(=O)[C@@H](C)Oc1ccc(Oc2ncc(Cl)cc2F)cc1]

Clodinafop can be applied to vegetation or an area adjacent the vegetation or applied to soil or water to prevent the emergence or growth of vegetation in an amount sufficient to induce a herbicidal effect. In some aspects, clodinafop is applied to vegetation or an area adjacent the vegetation or applied to soil or water to prevent the emergence or growth of vegetation in an amount of 10 g ai/ha or more, such as 11 g ai/ha or more, 12 g ai/ha or more, 13 g ai/ha or more, 14 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, or 95 g ai/ha or more; in an amount of 100 g ai/ha or less, such as 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 14 g ai/ha or less, 13 g ai/ha or less, 12 g ai/ha or less, or 11 g ai/ha or less; or in an amount ranging from any of the minimum values described above to any of the maximum values described above, such as 10-100 g ai/ha, 15-90 g ai/ha, 12-80 g ai/ha, 25-70 g ai/ha, 14-40 g ai/ha, 20-85 g ai/ha, 30-60 g ai/ha, 40-90 g ai/ha, 50-100 g ai/ha, 20-90 g ai/ha, 50-95 g ai/ha, 30-90 g ai/ha, 20-60 g ai/ha, 30-80 g ai/ha, 13-75 g ai/ha, or 11-95 g ai/ha.

### Cloproxydim

Compositions and methods of the present disclosure can include cloproxydim or an agriculturally acceptable salt or ester thereof. Cloproxydim, shown below, is a cyclohexanedione herbicide that has been evaluated by the Chevron Chemical Company. Cloproxydim provides post-emergence control of a wide range of annual and perennial grasses in beans, cotton, potatoes, sugar beets, and most vegetable crops. Its herbicidal activity is described in The Pesticide Manual, Seventeenth Edition, 2016.

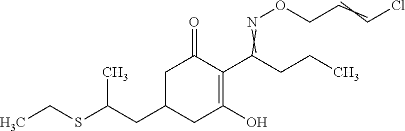 [SMILES: CCCC(=NOCC=CCl)C1=C(O)CC(CC(C)SCC)CC1=O]

### Cycloxydim

Compositions and methods of the present disclosure can include cycloxydim or an agriculturally acceptable salt or ester thereof. Cycloxydim, shown below, is a cyclohexanedione herbicide that provides post-emergence control of annual and perennial grasses (except red fescue and Poa spp.) in canola/oilseed rape, beans, potatoes, brassicas, cotton, celery, fennel, beetroot, sugar beets, fodder beets, sunflowers, soybeans, flax, alfalfa, crucifers, and alliums. Cycloxydim, as well as methods of preparing cycloxydim, are known in the art. Its herbicidal activity is described in The Pesticide Manual, Seventeenth Edition, 2016.

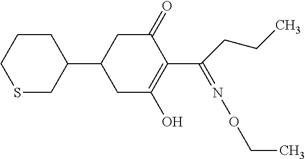 [SMILES: CCCC(=NOCC)C1=C(O)CC(C2CCCSC2)CC1=O]

Cycloxydim can be applied to vegetation or an area adjacent the vegetation or applied to soil or water to prevent the emergence or growth of vegetation in an amount sufficient to induce a herbicidal effect. In some aspects, cycloxydim is applied to vegetation or an area adjacent the vegetation or applied to soil or water to prevent the emergence or growth of vegetation in an amount of 50 g ai/ha or more, such as 55 g ai/ha or more, 60 g ai/ha or more, 70 g ai/ha or more, 80 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, or 675 g ai/ha or more; in an amount of 700 g ai/ha or less, such as 675 g ai/ha or less, 650 g ai/ha or less, 640 g ai/ha or less, 620 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 195 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 145 g ai/ha or less, 140 g ai/ha or less, 135 g ai/ha or less, 130 g ai/ha or less, 125 g ai/ha or less, 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-700 g ai/ha, 60-600 g ai/ha, 90-400 g ai/ha, 100-580 g ai/ha, 50-300 g ai/ha, 75-460 g ai/ha, 110-620 g ai/ha, 190-280 g ai/ha, 140-400 g ai/ha, 95-660 g ai/ha, 80-580 g ai/ha, 200-700 g ai/ha, 250-300 g ai/ha, 65-400 g ai/ha, 90-320 g ai/ha, or 55-675 g ai/ha.

### Cyhalofop

Compositions and methods of the present disclosure can include cyhalofop or an agriculturally acceptable salt or ester thereof. Cyhalofop, as well as methods of preparing cyhalofop, are known in the art. Cyhalofop is an aryloxyphenoxypropionate ester that is used for post-emergence control of grass weeds in rice. Cyhalofop-butyl, shown below, is an ester of cyhalofop.

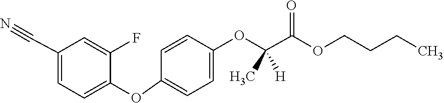 [SMILES: CCCCOC(=O)[C@@H](C)Oc1ccc(Oc2ccc(C#N)cc2F)cc1]

Cyhalofop can be applied to vegetation or an area adjacent the vegetation or applied to soil or water to prevent the emergence or growth of vegetation in an amount sufficient to induce a herbicidal effect. In some aspects, cyhalofop is applied to vegetation or an area adjacent the vegetation or applied to soil or water to prevent the emergence or growth of vegetation in an amount of 20 g ai/ha or more, such as 25 g ai/ha or more, 30 g ai/ha or more, 35 g ai/ha or more, 40 g ai/ha or more, 50 g ai/ha or more, 60 g ai/ha or more, 70 g ai/ha or more, 80 g ai/ha or more, 90 g ai/ha or more, 100 g ai/ha or more, 120 g ai/ha or more, 140 g ai/ha or more, 150 g ai/ha or more, 160 g ai/ha or more, 180 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, or 500 g ai/ha or more; in an amount of 500 g ai/ha or less, such as 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 180 g ai/ha or less, 160 g ai/ha or less, 150 g ai/ha or less, 140 g ai/ha or less, 120 g ai/ha or less, 100 g ai/ha or less, 90 g ai/ha or less, 80 g ai/ha or less, 70 g ai/ha or less, 60 g ai/ha or less, 50 g ai/ha or less, 40 g ai/ha or less, 35 g ai/ha or less, 30 g ai/ha or less, or 25 g ai/ha or less; or in an amount ranging from any of the minimum values described above to any of the maximum values described above, such as 20-500 g ai/ha, 20-450 g ai/ha, 25-200 g ai/ha, 40-400 g ai/ha, 60-375 g ai/ha, 40-350 g ai/ha, 50-250 g ai/ha, 60-325 g ai/ha, 90-275 g ai/ha, 70-325 g ai/ha, 40-225 g ai/ha, 140-300 g ai/ha, 35-160 g ai/ha, 80-350 g ai/ha, 150-450 g ai/ha, 200-425 g ai/ha, or 25-475 g ai/ha.

### Diclofop

Compositions and methods of the present disclosure can include diclofop or an agriculturally acceptable salt or ester thereof. Diclofop-methyl, shown below, is an aryloxyphenoxypropionate that is used for post-emergence control of wild oats, wild millets, and other annual grass weeds in wheat, barley, rye, red fescue, and broad-leaved crops such as soybeans, sugar beets, fodder beets, flax, legumes, canola/oilseed rape, sunflowers, clover, alfalfa, peanuts, brassicas, carrots, celery, parsnips, lettuce, spinach, potatoes, cucumbers, peas, beans, tomatoes, fennel, alliums, and herbs. Diclofop, as well as methods of preparing diclofop, are known in the art.

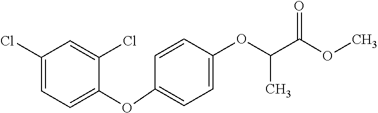 [SMILES: COC(=O)C(C)Oc1ccc(Oc2ccc(Cl)cc2Cl)cc1]

Diclofop can be applied to vegetation or an area adjacent the vegetation or applied to soil or water to prevent the emergence or growth of vegetation in an amount sufficient to induce a herbicidal effect. In some aspects, diclofop is applied to vegetation or an area adjacent the vegetation or applied to soil or water to prevent the emergence or growth of vegetation in an amount 100 g ai/ha or more, such as 150 g ai/ha or more, 175 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 620 g ai/ha or more, 640 g ai/ha or more, 660 g ai/ha or more, 680 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, or 1200 g ai/ha or more; in an amount of 1200 g ai/ha or less, such as 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 680 g ai/ha or less, 660 g ai/ha or less, 640 g ai/ha or less, 620 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 280 g ai/ha or less, 260 g ai/ha or less, 240 g ai/ha or less, 220 g ai/ha or less, 200 g ai/ha or less, 175 g ai/ha or less, 150 g ai/ha or less, 140 g ai/ha or less, 130 g ai/ha or less, 120 g ai/ha or less, 110 g ai/ha or less, or 101 g ai/ha or less; or in an amount ranging from any of the minimum values described above to any of the maximum values described above, such as 100-1200 g ai/ha, 120-1200 g ai/ha, 140-1100 g ai/ha, 150-900 g ai/ha, 175-1000 g ai/ha, 200-1150 g ai/ha, 360-1050 g ai/ha, 220-640 g ai/ha, 240-800 g ai/ha, 110-700 g ai/ha, 300-440 g ai/ha, 750-1000 g ai/ha, 360-950 g ai/ha, 175-850 g ai/ha, 400-650 g ai/ha, 150-280 g ai/ha, 500-600 g ai/ha, or 110-1150 g ai/ha).

### Fenoxaprop

Compositions and methods of the present disclosure can include fenoxaprop or an agriculturally acceptable salt or ester thereof. Fenoxaprop is an aryloxyphenoxypropionate that is used for post-emergence control of annual and perennial grass weeds in potatoes, beans, soybeans, beets, vegetables, peanuts, and cotton; and (when applied with the herbicide safener fenchlorazole-ethyl) annual and perennial grass weeds and wild oats in wheat, rye, and triticale. Fenoxaprop-P-ethyl, shown below, is an ester of fenoxaprop. Fenoxaprop, as well as methods of preparing fenoxaprop, are known in the art.

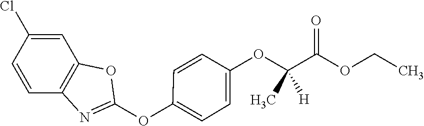 [SMILES: CCOC(=O)[C@@H](C)Oc1ccc(Oc2nc3ccc(Cl)cc3o2)cc1]

Fenoxaprop can be applied to vegetation or an area adjacent the vegetation or applied to soil or water to prevent the emergence or growth of vegetation in an amount sufficient to induce a herbicidal effect. In some aspects, fenoxaprop is applied to vegetation or an area adjacent the vegetation or applied to soil or water to prevent the emergence or growth of vegetation in an amount of 25 g ai/ha or more, such as 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 105 g ai/ha or more, 110 g ai/ha or more, 115 g ai/ha or more, 120 g ai/ha or more, 125 g ai/ha or more, 130 g ai/ha or more, 135 g ai/ha or more, 140 g ai/ha or more, 145 g ai/ha or more, or 150 g ai/ha or more; in an amount of 151 g ai/ha or less, such as 150 g ai/ha or less, 145 g ai/ha or less, 140 g ai/ha or less, 135 g ai/ha or less, 130 g ai/ha or less, 125 g ai/ha or less, 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 25 g ai/ha or less; or in an amount ranging from any of the minimum values described above to any of the maximum values described above, such as 25-151 g ai/ha, 30-150 g ai/ha, 35-140 g ai/ha, 50-130 g ai/ha, 30-125 g ai/ha, 85-150 g ai/ha, 45-145 g ai/ha, 25-110 g ai/ha, 40-90 g ai/ha, 55-120 g ai/ha, 40-115 g ai/ha, 60-110 g ai/ha, 30-105 g ai/ha, 90-151 g ai/ha, 25-95 g ai/ha, 50-80 g ai/ha, 60-125 g ai/ha, or 25-145 g ai/ha.

### Fenthiaprop

Compositions and methods of the present disclosure can include fenthiaprop or an agriculturally acceptable salt or ester thereof. Fenthiaprop, shown below, is an aryloxyphenoxypropionate herbicide that is used for post-emergence control of many annual and perennial grasses and volunteer cereals in canola/oilseed rape, potatoes, and sugar beets.

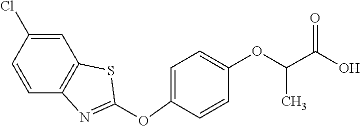 [SMILES: CC(Oc1ccc(Oc2nc3ccc(Cl)cc3s2)cc1)C(=O)O]

Fenthiaprop can be applied to vegetation or an area adjacent the vegetation or applied to soil or water to prevent the emergence or growth of vegetation in an amount sufficient to induce a herbicidal effect. In some aspects, fenthiaprop is applied to vegetation or an area adjacent the vegetation or applied to soil or water to prevent the emergence or growth of vegetation in an amount 150 g ai/ha or more, such as 160 g ai/ha or more, 170 g ai/ha or more, 180 g ai/ha or more, 190 g ai/ha or more, 200 g ai/ha or more, 220 g ai/ha or more, 240 g ai/ha or more, 250 g ai/ha or more, 260 g ai/ha or more, 280 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 420 g ai/ha or more, 440 g ai/ha or more, 450 g ai/ha or more, 460 g ai/ha or more, 480 g ai/ha or more, 500 g ai/ha or more, 520 g ai/ha or more, 540 g ai/ha or more, 550 g ai/ha or more, 560 g ai/ha or more, 580 g ai/ha or more, 600 g ai/ha or more, 620 g ai/ha or more, 640 g ai/ha or more, 650 g ai/ha or more, 660 g ai/ha or more, 680 g ai/ha or more, 700 g ai/ha or more, or 725 g ai/ha or more; in an amount of 750 g ai/ha or less, such as 725 g ai/ha or less, 700 g ai/ha or less, 680 g ai/ha or less, 660 g ai/ha or less, 650 g ai/ha or less, 640 g ai/ha or less, 620 g ai/ha or less, 600 g ai/ha or less, 580 g ai/ha or less, 560 g ai/ha or less, 550 g ai/ha or less, 540 g ai/ha or less, 520 g ai/ha or less, 500 g ai/ha or less, 480 g ai/ha or less, 460 g ai/ha or less, 450 g ai/ha or less, 440 g ai/ha or less, 420 g ai/ha or less, 400 g ai/ha or less, 380 g ai/ha or less, 360 g ai/ha or less, 350 g ai/ha or less, 340 g ai/ha or less, 320 g ai/ha or less, 300 g ai/ha or less, 280 g ai/ha or less, 260 g ai/ha or less, 250 g ai/ha or less, 240 g ai/ha or less, 220 g ai/ha or less, 200 g ai/ha or less, 190 g ai/ha or less, 180 g ai/ha or less, 170 g ai/ha or less, or 160 g ai/ha or less; or in an amount ranging from any of the minimum values described above to any of the maximum values described above, such as 150-750 g ai/ha, 180-600 g ai/ha, 170-650 g ai/ha, 250-700 g ai/ha, 280-450 g ai/ha, 360-580 g ai/ha, 220-640 g ai/ha, 240-725 g ai/ha, 340-440 g ai/ha, 260-550 g ai/ha, 170-250 g ai/ha, 400-650 g ai/ha, 190-580 g ai/ha, 500-600 g ai/ha, or 160-725 g ai/ha.

### Fluazifop

Compositions and methods of the present disclosure can include fluazifop or an agriculturally acceptable salt or ester thereof. Fluazifop is an aryloxyphenoxypropionate that is used for post-emergence control of annual and perennial grass weeds in broad-leaved crops, including control of volunteer cereals and other grass weeds in canola/oilseed rape, sugar beet, fodder beet, potatoes, cotton, soybeans, peanuts, pome fruit, bush fruit, vines, citrus fruit, pineapples, bananas, strawberries, sunflowers, alfalfa, coffee, ornamentals and many vegetables. Fluazifop-P-butyl, shown below, is an ester of fluazifop. Fluazifop, as well as methods of preparing fluazifop, are known in the art.

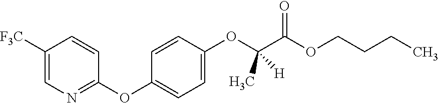 [SMILES: CCCCOC(=O)[C@@H](C)Oc1ccc(Oc2ccc(C(F)(F)F)cn2)cc1]

Fluazifop can be applied to vegetation or an area adjacent the vegetation or applied to soil or water to prevent the emergence or growth of vegetation in an amount sufficient to induce a herbicidal effect. In some aspects, fluazifop is applied to vegetation or an area adjacent the vegetation or applied to soil or water to prevent the emergence or growth of vegetation in an amount of 100 grams active ingredient per hectare (g ai/ha) or more, such as 105 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, or 1350 g ai/ha or more; in an amount of 1400 g ai/ha or less, such as 1350 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less or 105 g ai/ha or less; or in an amount ranging from any of the minimum values described above to any of the maximum values described above, e.g., 100-1400 g ai/ha, 140-1300 g ai/ha, 180-1200 g ai/ha, 200-950 g ai/ha, 300-800 g ai/ha, 250-700 g ai/ha, 130-450 g ai/ha, 100-500 g ai/ha, 190-850 g ai/ha, 110-375 g ai/ha, 225-475 g ai/ha, 130-600 g ai/ha, 150-1200 g ai/ha, 190-700 g ai/ha, 175-350 g ai/ha, 500-1100 g ai/ha, or 105-1350 g ai/ha.

### Haloxyfop

Compositions and methods of the present disclosure can include haloxyfop or an agriculturally acceptable salt or ester thereof. Haloxyfop is an aryloxyphenoxypropionate that is used post-emergence for control of annual and perennial grasses in sugar beets, fodder beets, canola/oilseed rape, potatoes, leafy vegetables, onions, flax, sunflowers, soybeans, vines, strawberries and other crops. Haloxyfop-P-methyl, shown below, is an ester of haloxyfop; haloxyfop-R-methyl is the racemic form of this herbicide. Haloxyfop, as well as methods of preparing haloxyfop, are known in the art.

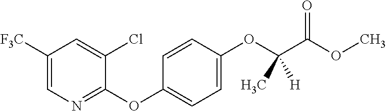 [SMILES: COC(=O)[C@@H](C)Oc1ccc(Oc2ncc(C(F)(F)F)cc2Cl)cc1]

Haloxyfop can be applied to vegetation or an area adjacent the vegetation or applied to soil or water to prevent the emergence or growth of vegetation in an amount sufficient to induce a herbicidal effect. In some aspects, haloxyfop is applied to vegetation or an area adjacent the vegetation or applied to soil or water to prevent the emergence or growth of vegetation in an amount of 24 g ai/ha or more, such as 25 g ai/ha or more, 30 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 210 g ai/ha or more, 220 g ai/ha or more, 230 g ai/ha or more, 240 g ai/ha or more, or 245 g ai/ha or more; in an amount of 250 g ai/ha or less, such as 245 g ai/ha or less, 240 g ai/ha or less, 230 g ai/ha or less, 220 g ai/ha or less, 210 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 25 g ai/ha or less; in an amount ranging from any of the minimum values described above to any of the maximum values described above, such as 24-250 g ai/ha, 25-225 g ai/ha, 35-200 g ai/ha, 55-190 g ai/ha, 25-180 g ai/ha, 30-175 g ai/ha, 30-230 g ai/ha, 130-220 g ai/ha, 60-160 g ai/ha, 75-150 g ai/ha, 35-150 g ai/ha, 60-140 g ai/ha, 80-230 g ai/ha, 90-210 g ai/ha, 55-110 g ai/ha, 70-240 g ai/ha, or 25-245 g ai/ha.

### Metamifop

Compositions and methods of the present disclosure can include metamifop. Metamifop, shown below, is an aryloxyphenoxypropionate that is used for post-emergence control of annual and perennial grass weeds (including Echinochloa spp., Leptochloa chinensis, Digitaria spp. and Eleusine indica) in rice, wheat, soybeans, cotton, and turf grass. Metamifop, as well as methods of preparing metamifop, are known in the art.

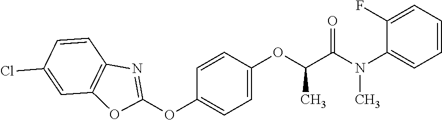 [SMILES: C[C@@H](Oc1ccc(Oc2nc3ccc(Cl)cc3o2)cc1)C(=O)N(C)c1ccccc1F]

Metamifop can be applied to vegetation or an area adjacent the vegetation or applied to soil or water to prevent the emergence or growth of vegetation in an amount sufficient to induce a herbicidal effect. In some aspects, metamifop is applied to vegetation or an area adjacent the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 105 g ai/ha or more, 110 g ai/ha or more, 115 g ai/ha or more, 120 g ai/ha or more, 125 g ai/ha or more, 130 g ai/ha or more, 135 g ai/ha or more, 140 g ai/ha or more, 145 g ai/ha or more, 150 g ai/ha or more, 155 g ai/ha or more, 160 g ai/ha or more, 165 g ai/ha or more, 170 g ai/ha or more, 175 g ai/ha or more, 180 g ai/ha or more, 185 g ai/ha or more, 190 g ai/ha or more, 195 g ai/ha or more, 200 g ai/ha or more, 210 g ai/ha or more, 220 g ai/ha or more, 230 g ai/ha or more, 240 g ai/ha or more, 250 g ai/ha or more, 260 g ai/ha or more, 270 g ai/ha or more, 280 g ai/ha or more, 290 g ai/ha or more, or 295 g ai/ha or more; in an amount of 300 g ai/ha or less, such as 295 g ai/ha or less, 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 195 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 145 g ai/ha or less, 140 g ai/ha or less, 135 g ai/ha or less, 130 g ai/ha or less, 125 g ai/ha or less, 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-300 g ai/ha, 60-275 g ai/ha, 80-250 g ai/ha, 55-125 g ai/ha, 60-200 g ai/ha, 70-160 g ai/ha, 80-210 g ai/ha, 90-270 g ai/ha, 100-175 g ai/ha, 120-230 g ai/ha, 145-250 g ai/ha, or 55-295 g ai/ha.

### Pinoxaden

Compositions and methods of the present disclosure can include pinoxaden or an agriculturally acceptable salt or ester thereof. Pinoxaden, shown below, is a phenylpyrazoline herbicide that provides post-emergence control of grass weeds in wheat, barley, lawns, and turf. Pinoxaden, as well as methods of preparing pinoxaden, are known in the art. Its herbicidal activity is described in The Pesticide Manual, Seventeenth Edition, 2016.

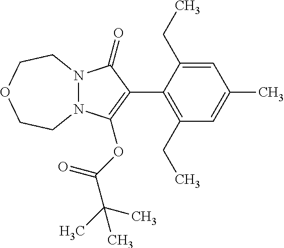 [SMILES: CCc1cc(C)cc(CC)c1-c1c(OC(=O)C(C)(C)C)n2n(c1=O)CCOCC2]

Pinoxaden can be applied to vegetation or an area adjacent the vegetation or applied to soil or water to prevent the emergence or growth of vegetation in an amount sufficient to induce a herbicidal effect. In some aspects, pinoxaden is applied to vegetation or an area adjacent the vegetation or applied to soil or water to prevent the emergence or growth of vegetation in an amount of 2 g ai/ha or more, such as 2.5 g ai/ha or more, 3 g ai/ha or more, 4 g ai/ha or more, 5 g ai/ha or more, 6 g ai/ha or more, 8 g ai/ha or more, 10 g ai/ha or more, 12 g ai/ha or more, 14 g ai/ha or more, 16 g ai/ha or more, 18 g ai/ha or more, 20 g ai/ha or more, 22 g ai/ha or more, 24 g ai/ha or more, 26 g ai/ha or more, 28 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 80 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, or 490 g ai/ha or more; in an amount of 500 g ai/ha or less, such as 490 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 235 g ai/ha or less, 230 g ai/ha or less, 225 g ai/ha or less, 220 g ai/ha or less, 215 g ai/ha or less, 210 g ai/ha or less, 205 g ai/ha or less, 200 g ai/ha or less, 195 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 80 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 28 g ai/ha or less, 26 g ai/ha or less, 24 g ai/ha or less, 22 g ai/ha or less, 20 g ai/ha or less, 18 g ai/ha or less, 16 g ai/ha or less, 14 g ai/ha or less, 12 g ai/ha or less, 10 g ai/ha or less, 8 g ai/ha or less, 6 g ai/ha or less, 5 g ai/ha or less, 4 g ai/ha or less, 3 g ai/ha or less, or 2.5 g ai/ha or less; or in an amount ranging from any of the minimum values described above to any of the maximum values described above, such as 2-500 g ai/ha, 3-400 g ai/ha, 5-300 g ai/ha, 8-100 g ai/ha, 5-500 g ai/ha, 10-120 g ai/ha, 22-190 g ai/ha, 40-300 g ai/ha, 30-400 g ai/ha, 80-270 g ai/ha, 100-480 g ai/ha, 150-250 g ai/ha, 110-380 g ai/ha, 8-235 g ai/ha, 30-260 g ai/ha, 24-50 g ai/ha, 10-300 g ai/ha, 16-180 g ai/ha, or 2.5-490 g ai/ha.

### Profoxydim

Compositions and methods of the present disclosure can include profoxydim or an agriculturally acceptable salt or ester thereof. Profoxydim, shown below, is a cyclohexanedione herbicide. Profoxydim is used for control of grass weeds, including Echinochloa spp., Brachiaria spp., Digitaria spp., Eleusine spp., Leptochloa spp., Setaria spp., Ischaemum rugosum and Rottboellia exaltata, in rice. Profoxydim, as well as methods of preparing profoxydim, are known in the art. Its herbicidal activity is described in The Pesticide Manual, Seventeenth Edition, 2016.

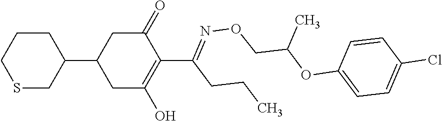 [SMILES: CCC/C(=N\OCC(C)Oc1ccc(Cl)cc1)C1=C(O)CC(C2CCCSC2)CC1=O]

Profoxydim can be applied to vegetation or an area adjacent the vegetation or applied to soil or water to prevent the emergence or growth of vegetation in an amount sufficient to induce a herbicidal effect. In some aspects, profoxydim is applied to vegetation or an area adjacent the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 105 g ai/ha or more, 110 g ai/ha or more, 115 g ai/ha or more, 120 g ai/ha or more, 125 g ai/ha or more, 130 g ai/ha or more, 135 g ai/ha or more, 140 g ai/ha or more, 145 g ai/ha or more, 150 g ai/ha or more, 155 g ai/ha or more, 160 g ai/ha or more, 165 g ai/ha or more, 170 g ai/ha or more, 175 g ai/ha or more, 180 g ai/ha or more, 185 g ai/ha or more, 190 g ai/ha or more, 195 g ai/ha or more, 200 g ai/ha or more, 210 g ai/ha or more, 220 g ai/ha or more, 230 g ai/ha or more, 240 g ai/ha or more, 250 g ai/ha or more, 260 g ai/ha or more, 270 g ai/ha or more, 280 g ai/ha or more, or 290 g ai/ha or more; in an amount of 300 g ai/ha or less (e.g., 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 195 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 145 g ai/ha or less, 140 g ai/ha or less, 135 g ai/ha or less, 130 g ai/ha or less, 125 g ai/ha or less, 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-300 g ai/ha., 60-275 g ai/ha, 85-250 g ai/ha, 60-220 g ai/ha, 120-280 g ai/ha, 150-180 g ai/ha, 70-160 g ai/ha, 55-240 g ai/ha, 90-290 g ai/ha, 75-100 g ai/ha, 115-270 g ai/ha, 70-200 g ai/ha, or 55-290 g ai/ha.

### Propaquizafop

Compositions and methods of the present disclosure can include propaquizafop. Propaquizafop, shown below, is an aryloxyphenoxypropionate that is to control a wide range of annual and perennial grasses in soybeans, cotton, sugar beets, potatoes, peanuts, peas, canola/oilseed rape and vegetables. Propaquizafop, as well as methods of preparing propaquizafop, are known in the art.

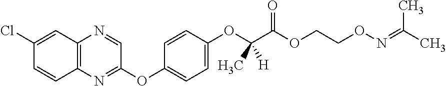 [SMILES: CC(C)=NOCCOC(=O)[C@@H](C)Oc1ccc(Oc2cnc3cc(Cl)ccc3n2)cc1]

Propaquizafop can be applied to vegetation or an area adjacent the vegetation or applied to soil or water to prevent the emergence or growth of vegetation in an amount sufficient to induce a herbicidal effect. In some aspects, propaquizafop is applied to vegetation or an area adjacent the vegetation or applied to soil or water to prevent the emergence or growth of vegetation in an amount of 29 g ai/ha or more, such as 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 210 g ai/ha or more, 220 g ai/ha or more, 230 g ai/ha or more, 240 g ai/ha or more, 250 g ai/ha or more, 260 g ai/ha or more, 270 g ai/ha or more, 280 g ai/ha or more, or 290 g ai/ha or more; in an amount of 300 g ai/ha or less (e.g., 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, or 30 g ai/ha or less; or in an amount ranging from any of the minimum values described above to any of the maximum values described above, such as 29-300 g ai/ha, 30-290 g ai/ha, 35-180 g ai/ha, 40-270 g ai/ha, 45-200 g ai/ha, 50-250 g ai/ha, 60-140 g ai/ha, 80-230 g ai/ha, 40-220 g ai/ha, 65-290 g ai/ha, 35-200 g ai/ha, 80-180 g ai/ha, 60-260 g ai/ha, 30-120 g ai/ha, 80-190 g ai/ha, 160-270 g ai/ha, or 30-290 g ai/ha.

### Quizalofop

Compositions and methods of the present disclosure can include quizalofop or an agriculturally acceptable salt or ester thereof. Quizalofop is an aryloxyphenoxypropionate that is used for selective post-emergence control of annual and perennial grass weeds in potatoes, soybeans, sugar beets, peanuts, canola/oilseed rape, sunflowers, vegetables, cotton and flax. Quizalofop-P-ethyl, shown below, is an ester of quizalofop. Quizalofop, as well as methods of preparing quizalofop, are known in the art.

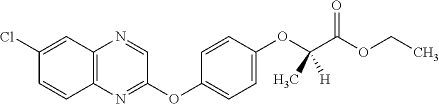 [SMILES: CCOC(=O)[C@@H](C)Oc1ccc(Oc2cnc3cc(Cl)ccc3n2)cc1]

Quizalofop can be applied to vegetation or an area adjacent the vegetation or applied to soil or water to prevent the emergence or growth of vegetation in an amount sufficient to induce a herbicidal effect. In some aspects, quizalofop is applied to vegetation or an area adjacent the vegetation or applied to soil or water to prevent the emergence or growth of vegetation in an amount of 24 g ai/ha or more, such as 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, or 245 g ai/ha or more; in an amount of 250 g ai/ha or less (e.g., 245 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 25 g ai/ha or less; or in an amount ranging from any of the minimum values described above to any of the maximum values described above, such as 24-250 g ai/ha, 25-225 g ai/ha, 55-200 g ai/ha, 40-190 g ai/ha, 25-80 g ai/ha, 60-175 g ai/ha, 110-225 g ai/ha, 75-160 g ai/ha, 45-220 g ai/ha, 80-150 g ai/ha, 30-90 g ai/ha, 40-100 g ai/ha, 50-180 g ai/ha, 60-230 g ai/ha, 60-120 g ai/ha, 40-100 g ai/ha, or 25-245 g ai/ha.

### Sethoxydim

Compositions and methods of the present disclosure can include sethoxydim or an agriculturally acceptable salt or ester thereof. Sethoxydim, shown below, is a cyclohexanedione herbicide that is used for controlling annual and perennial grasses in broad-leaved crops, including canola/oilseed rape, soybean, cotton, fodder beets, sunflowers, spinach, potatoes, tobacco, peanuts, strawberries, alfalfa, flax, vegetables, and sugar beet. Sethoxydim, as well as methods of preparing sethoxydim, are known in the art. Its herbicidal activity is described in The Pesticide Manual, Seventeenth Edition, 2016.

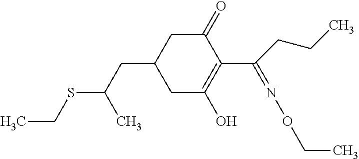 [SMILES: CCCC(=NOCC)C1=C(O)CC(CC(C)SCC)CC1=O]

Sethoxydim can be applied to vegetation or an area adjacent the vegetation or applied to soil or water to prevent the emergence or growth of vegetation in an amount sufficient to induce a herbicidal effect. In some aspects, sethoxydim is applied to vegetation or an area adjacent the vegetation or applied to soil or water to prevent the emergence or growth of vegetation in an amount of 2 g ai/ha or more, such as 3 g ai/ha or more, 4 g ai/ha or more, 5 g ai/ha or more, 6 g ai/ha or more, 8 g ai/ha or more, 10 g ai/ha or more, 12 g ai/ha or more, 14 g ai/ha or more, 16 g ai/ha or more, 18 g ai/ha or more, 20 g ai/ha or more, 22 g ai/ha or more, 24 g ai/ha or more, 26 g ai/ha or more, 28 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 80 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 620 g ai/ha or more, 640 g ai/ha or more, 660 g ai/ha or more, 680 g ai/ha or more, 700 g ai/ha or more, 750 g ai/ha or more, 800 g ai/ha or more, 850 g ai/ha or more, or 900 g ai/ha or more; in an amount of 900 g ai/ha or less, such as 850 g ai/ha or less, 800 g ai/ha or less, 750 g ai/ha or less, 700 g ai/ha or less, 680 g ai/ha or less, 660 g ai/ha or less, 640 g ai/ha or less, 620 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 235 g ai/ha or less, 230 g ai/ha or less, 225 g ai/ha or less, 220 g ai/ha or less, 215 g ai/ha or less, 210 g ai/ha or less, 205 g ai/ha or less, 200 g ai/ha or less, 195 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 80 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 28 g ai/ha or less, 26 g ai/ha or less, 24 g ai/ha or less, 22 g ai/ha or less, 20 g ai/ha or less, 18 g ai/ha or less, 16 g ai/ha or less, 14 g ai/ha or less, 12 g ai/ha or less, 10 g ai/ha or less, 8 g ai/ha or less, 6 g ai/ha or less, 5 g ai/ha or more, 4 g ai/ha or less, or 3 g ai/ha or less; or in an amount ranging from any of the minimum values described above to any of the maximum values described above, such as 2-900 g ai/ha, 3-500 g ai/ha, 5-270 g ai/ha, 10-600 g ai/ha, 22-440 g ai/ha, 35-560 g ai/ha, 10-400 g ai/ha, 20-700 g ai/ha, 40-280 g ai/ha, 60-680 g ai/ha, 100-750 g ai/ha, 90-300 g ai/ha, 200-700 g ai/ha, 300-500 g ai/ha, 3-100 g ai/ha, 5-90 g ai/ha, 12-105 g ai/ha, or 3-850 g ai/ha.

### Tepraloxydim

Compositions and methods of the present disclosure can include tepraloxydim or an agriculturally acceptable salt or ester thereof. Tepraloxydim, shown below, is a cyclohexanedione herbicide that is used for broad-spectrum post-emergence grass weed control, especially Poa annua and volunteer maize, and also Sorghum halepense and Elymus repens, in broad-leaved crops. Tepraloxydim, as well as methods of preparing tepraloxydim, are known in the art. Its herbicidal activity is described in The Pesticide Manual, Seventeenth Edition, 2016.

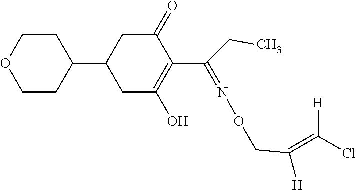 [SMILES: CCC(=NOC/C=C/Cl)C1=C(O)CC(C2CCOCC2)CC1=O]

Tepraloxydim can be applied to vegetation or an area adjacent the vegetation or applied to soil or water to prevent the emergence or growth of vegetation in an amount sufficient to induce a herbicidal effect. In some aspects, tepraloxydim is applied to vegetation or an area adjacent the vegetation or applied to soil or water to prevent the emergence or growth of vegetation in an amount of 25 g ai/ha or more, such as 27.5 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, or 195 g ai/ha or more; in an amount of 200 g ai/ha or less, such as 195 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 27.5 g ai/ha or less; or in an amount ranging from any of the minimum values described above to any of the maximum values described above, such as 25-200 g ai/ha, 25-190 g ai/ha, 60-180 g ai/ha, 35-170 g ai/ha, 45-120 g ai/ha, 25-65 g ai/ha, 90-160 g ai/ha, 75-195 g ai/ha, 25-150 g ai/ha, 30-190 g ai/ha, 35-130 g ai/ha, 50-120 g ai/ha, 65-180 g ai/ha, or 27.5-195 g ai/ha.

### Tralkoxydim

Compositions and methods of the present disclosure can include tralkoxydim or an agriculturally acceptable salt or ester thereof. Tralkoxydim is a cyclohexanedione herbicide that has the chemical name 2-[1-(ethoxyimino) propyl]-3-hydroxy-5-mesitylcyclohex-2-enone, and has the following structure:

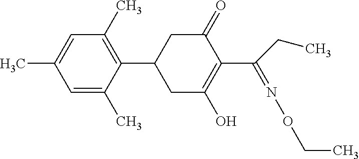 [SMILES: CCON=C(CC)C1=C(O)CC(c2c(C)cc(C)cc2C)CC1=O]

Tralkoxydim is used for post-emergence control of annual grass weeds (including Avena spp., Lolium spp., Setaria viridis, Phalaria spp., Alopecurus myosuroides, and Apera spica-venti) in wheat and barley. Tralkoxydim, as well as methods of preparing tralkoxydim, are known in the art. Its herbicidal activity is described in The Pesticide Manual, Seventeenth Edition, 2016.

Tralkoxydim can be applied to vegetation or an area adjacent the vegetation or applied to soil or water to prevent the emergence or growth of vegetation in an amount of 95 g ai/ha or more, such as 100 g ai/ha or more, 105 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, or 500 g ai/ha or more; in an amount of 501 g ai/ha or less, such as 500 g ai/ha or less, 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, or 100 g ai/ha or less; or in an amount ranging from any of the minimum values described above to any of the maximum values described above, such as 95-501 g ai/ha., 100-475 g ai/ha, 120-450 g ai/ha, 110-425 g ai/ha, 225-400 g ai/ha, 150-425 g ai/ha, 180-375 g ai/ha, 190-350 g ai/ha, 110-225 g ai/ha, 180-500 g ai/ha, or 100-500 g ai/ha.

### Plant Growth Regulators

In addition to the compounds according to Formula I or agriculturally acceptable N-oxide, salt or ester thereof, the compositions can include a plant growth regulator (PGR), an agriculturally acceptable salt or ester thereof, or mixtures thereof. PGRs, also called plant hormones, act as chemical messengers for intercellular communication. PGRs can be classified into a number of modes of action that may influence, for example, the growth, division, elongation, or differentiation of plant cells. Examples of PGRs include 1,4-dimethylnapththalene, 1-methylcyclopropene, 1-napthylacetic acid, 2,6-diisopropylnaphthalene, 2-naphthyloxyacetic acid, 4-chlorophenoxyacetic acid (4-CPA), 6-benzylaminopurine, abscisic acid, amidochlor, ancymidol, aviglycine, butralin, carbaryl, chlorflurenol, chlormequat, chlorphonium chloride, chlorpropham, clofencet, cloprop, cloxyfonac, cuprous chloride, cyanamide, cyclanilide, cycloheximide, cytokinins, daminozide, decan-1-ol, dikegulac, dimethipin, dimexano, endothal, etacelasil, ethephon, ethychlozate, fenoprop, fenridazon, flumetralin, flurenol, flurprimidol, forchlorfenuron, gibberellins, glyphosine, heptamaloxyloglucan, heptopargil, hexafluoroacetone trihydrate, inabenfide, indol-3-butyric acid (IBA), indol-3-ylacetic acid (IAA), isoprothiolane, maleic hydrazide, mefluidide, mepiquat, N-acetylthiazolidine-4-carboxylic acid, naphthaleneacetamide, N-m-tolylphthalamic acid, N-phenylphthalamic acid, nitrophenolates, paclobutrazol, pelargonic acid, piproctanyl bromide, prohexadione, prohydrojasmon, propham, propyl-3-tert-butylphenoxyacetate, sintofen, tetcyclacis, thidiazuron, triacontanol, triapenthenol, trinexapac, and uniconazole.

In some aspects, the composition can include a PGR selected from the group consisting of 1,4-dimethylnapththalene, 1-methylcyclopropene, 1-napthylacetic acid, 2,6-diisopropylnaphthalene, 2-naphthyloxyacetic acid, 4-chlorophenoxyacetic acid (4-CPA), 6-benzylaminopurine, abscisic acid, amidochlor, ancymidol, aviglycine, butralin, carbaryl, chlorflurenol, chlormequat, chlorphonium chloride, chlorpropham, clofencet, cloprop, cloxyfonac, cuprous chloride, cyanamide, cyclanilide, cycloheximide, cytokinins, daminozide, decan-1-ol, dikegulac, dimethipin, dimexano, endothal, etacelasil, ethephon, ethychlozate, fenoprop, fenridazon, flumetralin, flurenol, flurprimidol, forchlorfenuron, gibberellins, glyphosine, heptamaloxyloglucan, heptopargil, hexafluoroacetone trihydrate, inabenfide, indol-3-butyric acid (IBA), indol-3-ylacetic acid (IAA), isoprothiolane, maleic hydrazide, mefluidide, mepiquat, N-acetylthiazolidine-4-carboxylic acid, naphthaleneacetamide, N-m-tolylphthalamic acid, N-phenylphthalamic acid, nitrophenolates, paclobutrazol, pelargonic acid, piproctanyl bromide, prohexadione, prohydrojasmon, propham, propyl-3-tert-butylphenoxyacetate, sintofen, tetcyclacis, thidiazuron, triacontanol, triapenthenol, trinexapac, uniconazole, agriculturally acceptable salts or esters thereof, and mixtures thereof.

PGRs can be applied to vegetation or an area adjacent the vegetation or applied to soil or water to prevent the emergence or growth of vegetation in an amount sufficient to induce a herbicidal effect. In some aspects, the PGR is applied to vegetation or an area adjacent the vegetation or applied to soil or water to prevent the emergence or growth of vegetation in an amount of 20 g ai/ha or more, such as 0.020 g ai/ha or more, such as 0.025 g ai/ha or more, 0.03 g ai/ha or more, 0.04 g ai/ha or more, 0.05 g ai/ha or more, 0.06 g ai/ha or more, 0.07 g ai/ha or more, 0.08 g ai/ha or more, 0.09 g ai/ha or more, 0.1 g ai/ha or more, 0.11 g ai/ha or more, 0.125 g ai/ha or more, 0.15 g ai/ha or more, 0.175 g ai/ha or more, 0.20 g ai/ha or more, 0.25 g ai/ha or more, 0.3 g ai/ha or more, 0.4 g ai/ha or more, 0.5 g ai/ha or more, 0.6 g ai/ha or more, 0.7 g ai/ha or more, 0.8 g ai/ha or more, 0.9 g ai/ha or more, 1 g ai/ha or more, 1.1 g ai/ha or more, 1.25 g ai/ha or more, 1.5 g ai/ha or more, 1.75 g ai/ha or more, 2 g ai/ha or more, 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.25 g ai/ha or more, 4.5 g ai/ha or more, 4.75 g ai/ha or more, 5 g ai/ha or more, 5.25 g ai/ha or more, 5.5 g ai/ha or more, 5.75 g ai/ha or more, 6 g ai/ha or more, 6.25 g ai/ha or more, 6.5 g ai/ha or more, 6.75 g ai/ha or more, 7 g ai/ha or more, 7.25 g ai/ha or more, 7.5 g ai/ha or more, 8 g ai/ha or more, 8.5 g ai/ha or more, 9 g ai/ha or more, 9.5 g ai/ha, or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3750 g ai/ha or more, 4000 g ai/ha or more, 4250 g ai/ha or more, 4500 g ai/ha or more, 4750 g ai/ha or more, 5000 g ai/ha or more, 5250 g ai/ha or more, 5500 g ai/ha or more, 5750 g ai/ha or more, 6000 g ai/ha or more, 6250 g ai/ha or more, 6500 g ai/ha or more, 6750 g ai/ha or more, 7000 g ai/ha or more, 7250 g ai/ha or more, 7500 g ai/ha or more, 7750 g ai/ha or more, 8000 g ai/ha or more, 8250 g ai/ha or more, 8500 g ai/ha or more, 8750 g ai/ha or more, 9000 g ai/ha or more, 9250 g ai/ha or more, 9500 g ai/ha or more, 9750 g ai/ha or more, 10 kg ai/ha or more, 10.5 kg ai/ha or more, 11 kg ai/ha or more, 11.5 kg ai/ha or more, 12 kg ai/ha or more, 12.5 kg ai/ha or more, 13 kg ai/ha or more, 13.5 kg ai/ha or more, 14 kg ai/ha or more, 14.5 kg ai/ha or more, 15 kg ai/ha or more, 15.5 kg ai/ha or more, 16 kg ai/ha or more, 16.5 kg ai/ha or more, 16.75 kg ai/ha or more, 17 kg ai/ha or more, or 17.5 kg ai/ha or more; in an amount of 17.5 kg ai/ha or less, such as 17 kg ai/ha or less, 16.5 kg ai/ha or less, 16 kg ai/ha or less, 15.5 kg ai/ha or less, 15 kg ai/ha or less, 14.5 kg ai/ha or less, 14 kg ai/ha or less, 13.5 kg ai/ha or less, 13 kg ai/ha or less, 12.5 kg ai/ha or less, 12 kg ai/ha or less, 11.5 kg ai/ha or less, 11 kg ai/ha or less, 10.5 kg ai/ha, 10 kg ai/ha or less, 9750 g ai/ha or less, 9500 g ai/ha or less, 9250 g ai/ha or less, 9000 g ai/ha or less, 8750 g ai/ha or less, 8500 g ai/ha or less, 8250 g ai/ha or less, 8000 g ai/ha or less, 7750 g ai/ha or less, 7500 g ai/ha or less, 7250 g ai/ha or less, 7000 g ai/ha or less, 6750 g ai/ha or less, 6500 g ai/ha or less, 6250 g ai/ha or less, 6000 g ai/ha or less, 5750 g ai/ha or less, 5500 g ai/ha or less, 5250 g ai/ha or less, 5000 g ai/ha or less, 4750 g ai/ha or less, 4500 g ai/ha or less, 4250 g ai/ha or less, 4000 g ai/ha or less, 3750 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9.5 g ai/ha or less, 9 g ai/ha or less, 8.5 g ai/ha or less, 8 g ai/ha or less, 7.5 g ai/ha or less, 7.25 g ai/ha or less, 7 g ai/ha or less, 6.75 g ai/ha or less, 6.5 g ai/ha or less, 6.25 g ai/ha or less, 6 g ai/ha or less, 5.75 g ai/ha or less, 5.5 g ai/ha or less, 5.25 g ai/ha or less, 5 g ai/ha or less, 4.75 g ai/ha or less, 4.5 g ai/ha or less, 4.25 g ai/ha or less, 4 g ai/ha or less, 3.75 g ai/ha or less, 3.5 g ai/ha or less, 3.25 g ai/ha or less, 3 g ai/ha or less, 2.75 g ai/ha or less, 2.5 g ai/ha or less, 2.25 g ai/ha or less, 2 g ai/ha or less, 1.75 g ai/ha or less, 1.5 g ai/ha or less, 1.25 g ai/ha or less, 1.1 g ai/ha or less, 1 g ai/ha or less, 0.9 g ai/ha or less, 0.8 g ai/ha or less, 0.7 g ai/ha or less, 0.6 g ai/ha or less, 0.5 g ai/ha or less, 0.4 g ai/ha or less, 0.3 g ai/ha or less, 0.2 g ai/ha or less, 0.175 g ai/ha or less, 0.15 g ai/ha or less, 0.125 g ai/ha or less, 0.11 g ai/ha or less, 0.1 g ai/ha or less, 0.09 g ai/ha or less, 0.08 g ai/ha or less, 0.07 g ai/ha or less, 0.06 g ai/ha or less, 0.05 g ai/ha or less, 0.04 g ai/ha or less, 0.03 g ai/ha or less, or 0.025 g ai/ha or less; or in an amount ranging from any of the minimum values described above to any of the maximum values described above, such as 0.020 g-17.5 kg ai/ha, 950-6500 g ai/ha, 0.7-8.5 g ai/ha, 460-3750 g ai/ha, 600-2500 g ai/ha, 50-5000 g ai/ha, 110-450 g ai/ha, 11.5-16 kg ai/ha, 1900-4000 g ai/ha, 1-4 g ai/ha, 750-2200 g ai/ha, 380-5250 g ai/ha, 0.2-17.25 g ai/ha, 500-1400 g ai/ha, 3.25-950 g ai/ha, 440-2900 g ai/ha, or 0.025 g-17 kg ai/ha.

### 1,4-Dimethylnaphthalene

Compositions and methods of the present disclosure can include 1,4-dimethylnaphthalene or an agriculturally acceptable salt or ester thereof. 1,4-Dimethylnaphthalene, shown below, is an aromatic hydrocarbon that provides inhibition of sprouting in potatoes after packaging, during shipping and in storage or in seed potatoes, at a spray rate of 5-20 mg/kg of potatoes. 1,4-Dimethylnaphthalene, as well as methods of preparing 1,4-dimethylnaphthalene, are known in the art. Its plant growth regulation activity is described, for example, in The Pesticide Manual, Eighteenth Edition, 2016.

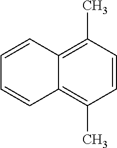

### 1-Methylcyclopropene

Compositions and methods of the present disclosure can include 1-methylcyclopropene or an agriculturally acceptable salt or ester thereof. 1-Methylcyclopropene, shown below, is a cyclic hydrocarbon that extends the life of cut and potted flowering plants and of harvested fruit and vegetables. It is formulated as a powder and released as a gas when the product is added to water. 1-Methylcyclopropene, as well as methods of preparing 1-methylcyclopropene, are known in the art. Its plant growth regulation activity is described, for example, in The Pesticide Manual, Eighteenth Edition, 2016.

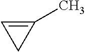

1-Methylcyclopropene can be applied to vegetation or an area adjacent the vegetation or applied to soil or water to prevent the emergence or growth of vegetation in an amount sufficient to induce a herbicidal effect. In some aspects, 1-methylcyclopropene is applied to vegetation or an area adjacent the vegetation or applied to soil or water to prevent the emergence or growth of vegetation in an amount of 5 g ai/ha or more, such as 5.25 g ai/ha or more, 5.5 g ai/ha or more, 5.75 g ai/ha or more, 6 g ai/ha or more, 6.25 g ai/ha or more, 6.5 g ai/ha or more, 6.75 g ai/ha or more, 7 g ai/ha or more, 7.25 g ai/ha or more, 7.5 g ai/ha or more, 8 g ai/ha or more, 8.5 g ai/ha or more, 9 g ai/ha or more, 9.5 g ai/ha, or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, or 47.5 g ai/ha or more; in an amount of such as 50 g ai/ha or less, such as 47.5 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9.5 g ai/ha or less, 9 g ai/ha or less, 8.5 g ai/ha or less, 8 g ai/ha or less 7.5 g ai/ha or less, 7.25 g ai/ha or less, 7 g ai/ha or less, 6.75 g ai/ha or less, 6.5 g ai/ha or less, 6.25 g ai/ha or less, 6 g ai/ha or less, 5.75 g ai/ha or less, 5.5 g ai/ha or less, 5.25 g ai/ha or less; or in an amount ranging from any of the minimum values described above to any of the maximum values described above, such as 15-50 g ai/ha, 9.5-30 g ai/ha, 5-25 g ai/ha, 20-45 g ai/ha, 5.25-9.5 g ai/ha, or 7.5-47.5 g ai/ha.

### 1-Naphthylacetic Acid

Compositions and methods of the present disclosure can include 1-naphthylacetic acid or an agriculturally acceptable salt or ester thereof. 1-Naphthylacetic acid, shown below, is a naphthylacetic acid that: stimulates root formation in cuttings of woody and herbaceous plants and vines at 0.1-0.6 g/plant; prevents premature flower and fruit drop in apples, pears, grapes, guavas, mangoes, watermelons, pawpaws, citrus, aubergines, cucumbers, cotton, soybeans, etc.; increases fruit setting; provides thinning of fruit in apples, pears, olives, citrus and other fruit; and induces flowering in pineapples. 1-Naphthylacetic acid, as well as methods of preparing 1-naphthylacetic acid, are known in the art. Exemplary salts of 1-Naphthylacetic acid include ammonium 1-Naphthylacetic acetate, sodium 1-Naphthylacetic acetate, and potassium 1-Naphthylacetic

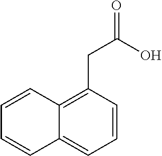 [SMILES: O=C(O)Cc1cccc2ccccc12]

acetate, while esters include ethyl 1-Naphthylacetic acetate. Its plant growth regulation activity is described, for example, in The Pesticide Manual, Eighteenth Edition, 2016.

1-Naphthylacetic acid can be applied to vegetation or an area adjacent the vegetation or applied to soil or water to prevent the emergence or growth of vegetation in an amount sufficient to induce a herbicidal effect. In some aspects, 1-naphthylacetic acid is applied to vegetation or an area adjacent the vegetation or applied to soil or water to prevent the emergence or growth of vegetation in an amount of 100 g ai/ha or more, such as 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, or 390 g ai/ha or more; in an amount of 400 g ai/ha or less, such as 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less; or in an amount ranging from any of the minimum values described above to any of the maximum values described above, such as 100-400 g ai/ha, 130-275 g ai/ha, 260-370 g ai/ha, 200-250 g ai/ha, 190-380 g ai/ha, 110-320 g ai/ha, 310-400 g ai/ha, 140-220 g ai/ha, 260-360 g ai/ha, or 110-390 g ai/ha.

### 2,6-Diisopropylnaphthalene

Compositions and methods of the present disclosure can include 2,6-diisopropylnaphthalene or an agriculturally acceptable salt or ester thereof. 2,6-Diisopropylnaphthalene, shown below, is an aromatic hydrocarbon that inhibits sprouting in potatoes, at a spray rate of 2.5-25 mg/kg of potatoes. 2,6-Diisopropylnaphthalene, as well as

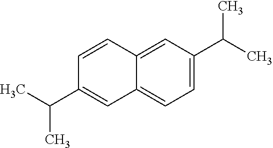 [SMILES: CC(C)c1ccc2cc(C(C)C)ccc2c1]

methods of preparing 2,6-diisopropylnaphthalene, are known in the art. Its plant growth regulation activity is described, for example, in The Pesticide Manual, Eighteenth Edition, 2016.

### 2-Naphthyloxyacetic Acid

Compositions and methods of the present disclosure can include 2-naphthyloxyacetic acid or an agriculturally acceptable salt or ester thereof. 2-Naphthyloxyacetic acid, shown below, is a phenoxycarboxylic acid that increases fruit setting in tomatoes, strawberries, blackberries, peppers, aubergines, grapes and pineapples. 2-Naphthyloxyacetic acid, as well as methods of preparing 2-naphthyloxyacetic acid, are known in the art. Its plant growth regulation activity is described, for example, in The Pesticide Manual, Eighteenth Edition, 2016.

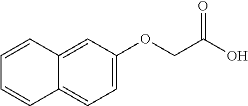 [SMILES: O=C(O)COc1ccc2ccccc2c1]

### 4-Chlorophenoxyacetic Acid (4-CPA)

Compositions and methods of the present disclosure can include 4-chlorophenoxyacetic acid (4-CPA) or an agriculturally acceptable salt or ester thereof. 4-CPA, shown below, is a phenoxycarboxylic acid that improves fruit setting in tomatoes, by application to the flowers, at 1.5-3 g/hl of spray solution; inhibition of sprout formation in mung beans; thinning of fruit in peaches. 4-CPA, as well as methods of preparing 4-CPA, are known in the art. Its plant growth regulation activity is described, for example, in The Pesticide Manual, Eighteenth Edition, 2016.

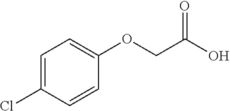 [SMILES: O=C(O)COc1ccc(Cl)cc1]

### 6-Benzylaminopurine

Compositions and methods of the present disclosure can include 6-benzylaminopurine or an agriculturally acceptable salt or ester thereof. 6-Benzylaminopurine, shown below, is a aminopurine that stimulates lateral bud emergence in apples, pears and oranges, formation of basal shoots in roses and orchids, flowering in cyclamens and cacti, fruit set in grapes, oranges and melons, fruit thinning, flower bud formation and regular bearing in tree fruit, inhibition of senescence in rice seedlings, enhancement of lateral bud break and shoot growth in white pine trees, uniform bolting and increased seed production in spinach, all by foliar application, soaking, or painting, at 75 g/ha in apples. 6-Benzylaminopurine, as well as methods of preparing 6-benzylaminopurine, are known in the art. Its plant growth regulation activity is described, for example, in The Pesticide Manual, Eighteenth Edition, 2016.

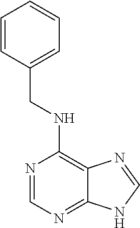 [SMILES: c1ccc(CNc2ncnc3[nH]cnc23)cc1]

6-Benzylaminopurine can be applied to vegetation or an area adjacent the vegetation or applied to soil or water to prevent the emergence or growth of vegetation in an amount sufficient to induce a herbicidal effect. In some aspects, 6-benzylaminopurine is applied to vegetation or an area adjacent the vegetation or applied to soil or water to prevent the emergence or growth of vegetation in an amount of 10 g ai/ha or more, such as 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, or 490 g ai/ha or more; in an amount of 500 g ai/ha or less, such as 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, or 15 g ai/ha or less; or in an amount ranging from any of the minimum values described above to any of the maximum values described above, such as 10-500 g ai/ha, 95-340 g ai/ha, 400-475 g ai/ha, 60-250 g ai/ha, 75-375 g ai/ha, 110-460 g ai/ha, 180-430 g ai/ha, 15-220 g ai/ha, 35-350 g ai/ha, 160-410 g ai/ha, 40-230 g ai/ha, or 15-490 g ai/ha.

### Abscisic Acid

Compositions and methods of the present disclosure can include abscisic acid or an agriculturally acceptable salt or ester thereof. Abscisic acid, shown below, is a plant hormone that reduces drought stress and controls wilting in ornamental plants; decreases water requirements during shipping of ornamental plants; accelerates and enhances the color development of grapes, using a spray solution at 7.5-50 g/hl concentration. Abscisic acid, as well as methods of preparing abscisic acid, are known in the art. Its plant growth regulation activity is described, for example, in The Pesticide Manual, Eighteenth Edition, 2016.

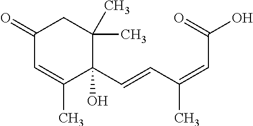 [SMILES: CC1=CC(=O)CC(C)(C)[C@@]1(O)/C=C/C(C)=C\C(=O)O]

Abscisic acid can be applied to vegetation or an area adjacent the vegetation or applied to soil or water to prevent the emergence or growth of vegetation in an amount sufficient to induce a herbicidal effect. In some aspects, abscisic acid is applied to vegetation or an area adjacent the vegetation or applied to soil or water to prevent the emergence or growth of vegetation in an amount of 75 g ai/ha or more, such as 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, or 950 g ai/ha or more; in an amount of 1000 g ai/ha or less, such as 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, or 80 g ai/ha or less; or in an amount ranging from any of the minimum values described above to any of the maximum values described above, such as 75-1000 g ai/ha, 95-650 g ai/ha, 460-850 g ai/ha, 220-575 g ai/ha, 450-500 g ai/ha, 110-325 g ai/ha, 330-490 g ai/ha, 750-950 g ai/ha, 380-700 g ai/ha, 500-1000 g ai/ha, 240-600 g ai/ha, or 80-950 g ai/ha.

### Ancymidol

Compositions and methods of the present disclosure can include ancymidol or an agriculturally acceptable salt or ester thereof. Ancymidol, shown below, is a pyrimidine carbinol used to reduce internode elongation to produce more compact greenhouse plants, by application to either foliage or soil, at 0.6-6.6 g/hl for bedding plants, 0.3-3.5 g/hl for bedding plant plugs, 2-5 g/hl for flowering and foliage plants, 2.5-5 g/hl for bulbs. Ancymidol, as well as methods of preparing ancymidol, are known in the art. Its plant growth regulation activity is described, for example, in The Pesticide Manual, Eighteenth Edition, 2016.

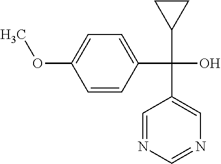 [SMILES: COc1ccc(C(O)(c2cncnc2)C2CC2)cc1]

### Aviglycine

Compositions and methods of the present disclosure can include aviglycine or an agriculturally acceptable salt or ester thereof. Aviglycine, shown below, is an amino acid used to reduce fruit drop, reduce delay of fruit maturity, reduce delay or extension of harvest, improve harvest management, maintain fruit quality (e.g. fruit firmness), enhance size and color as a result of delayed harvest, reduce incidence and/or severity of watercore and superficial scald, enhance storage potential in apples, pears, stone fruit and walnuts and increase nut set in walnut cultivars affected by pistillate flower abortion. Aviglycine, as well as methods of preparing aviglycine, are known in the art. Its plant growth regulation activity is described, for example, in The Pesticide Manual, Eighteenth Edition, 2016.

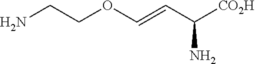 [SMILES: NCCO/C=C/[C@H](N)C(=O)O]

Aviglycine can be applied to vegetation or an area adjacent the vegetation or applied to soil or water to prevent the emergence or growth of vegetation in an amount sufficient to induce a herbicidal effect. In some aspects, aviglycine is applied to vegetation or an area adjacent the vegetation or applied to soil or water to prevent the emergence or growth of vegetation in an amount of 200 g ai/ha or more, such as 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, or 725 g ai/ha or more; in an amount of 750 g ai/ha or less, such as 725 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, or 210 g ai/ha or less; or in an amount ranging from any of the minimum values described above to any of the maximum values described above, such as 200-750 g ai/ha, 275-525 g ai/ha, 460-550 g ai/ha, 300-700 g ai/ha, 210-450 g ai/ha, 400-600 g ai/ha, 330-725 g ai/ha, 380-550 g ai/ha, 425-575 g ai/ha, 440-750 g ai/ha, or 210-725 g ai/ha.

### Butralin

Compositions and methods of the present disclosure can include butralin or an agriculturally acceptable salt or ester thereof. Butralin, shown below, is a dinitroaniline used for pre-emergence control of annual broad-leaved and grass weeds in cotton, soybeans, rice, barley, beans, alliums, vines, ornamentals, fruit and nut trees, at 1120-3400 g/ha; control of suckers in tobacco, at 125 mg/plant. Butralin, as well as methods of preparing butralin, are known in the art. Its plant growth regulation activity is described, for example, in The Pesticide Manual, Eighteenth Edition, 2016.

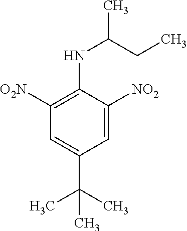 [SMILES: CCC(C)Nc1c([N+](=O)[O-])cc(C(C)(C)C)cc1[N+](=O)[O-]]

Butralin can be applied to vegetation or an area adjacent the vegetation or applied to soil or water to prevent the emergence or growth of vegetation in an amount sufficient to induce a herbicidal effect. In some aspects, butralin is applied to vegetation or an area adjacent the vegetation or applied to soil or water to prevent the emergence or growth of vegetation in an amount of 1100 g ai/ha or more, such as 1120 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more or 3400 g ai/ha or more; in an amount of 3500 g ai/ha or less, such as 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, or 1120 g ai/ha or less; or in an amount ranging from any of the minimum values described above to any of the maximum values described above, such as 1100-3500 g ai/ha, 1450-2900 g ai/ha, 1300-3200 g ai/ha, 1600-2500 g ai/ha, 1250-3000 g ai/ha, 1800-2400 g ai/ha, 1900-3400 g ai/ha, 1350-2700 g ai/ha, 1120-3100 g ai/ha, 1500-3500 g ai/ha, 2200-2900 g ai/ha, or 1120-3400 g ai/ha.

### Chlorflurenol

Compositions and methods of the present disclosure can include chlorflurenol or an agriculturally acceptable salt or ester thereof. Chlorflurenol, shown below, is a polycyclic aromatic carboxylic acid used as a growth retardant, at 2000-4000 g/ha, and for weed suppression, at 500-1500 g/ha as a soil application, in amenity grass, roadsides, railways, ditch banks, etc. Chlorflurenol also increases yield and improve size grading in pickling cucumbers, and induces vegetative planting material in pineapples. Chlorflurenol, as well as methods of preparing chlorflurenol, are known in the art. Its plant growth regulation activity is described, for example, in The Pesticide Manual, Eighteenth Edition, 2016.

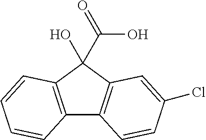 [SMILES: O=C(O)C1(O)c2ccccc2-c2ccc(Cl)cc21]

Chlorflurenol can be applied to vegetation or an area adjacent the vegetation or applied to soil or water to prevent the emergence or growth of vegetation in an amount sufficient to induce a herbicidal effect. In some aspects, chlorflurenol is applied to vegetation or an area adjacent the vegetation or applied to soil or water to prevent the emergence or growth of vegetation in an amount of 500 g ai/ha or more, such as 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3250 g ai/ha or more, 3500 g ai/ha or more, or 3750 g ai/ha or more; in an amount of 4000 g ai/ha or less, such as 3750 g ai/ha or less, 3500 g ai/ha or less, 3250 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, or 525 g ai/ha or less; or in an amount ranging from any of the minimum values described above to any of the maximum values described above, such as 500-4000 g ai/ha, 950-2500 g ai/ha, 675-3250 g ai/ha, 800-2100 g ai/ha, 550-2000 g ai/ha, 1100-2700 g ai/ha, 1900-2600 g ai/ha, 750-1200 g ai/ha, 3000-3250 g ai/ha, 600-1400 g ai/ha, 850-2300 g ai/ha, or 525-3750 g ai/ha.

### Chlormequat Chloride

Compositions and methods of the present disclosure can include chlormequat chloride. Chlormequat chloride, shown below, is a quaternary ammonium salt used to: increase resistance to lodging and increase yields in wheat, rye, oats and triticale, at 800-1600 g/ha; promote lateral branching and flowering in azaleas, fuchsias, begonias, poinsettias, geraniums, pelargoniums and other ornamental plants; promote flower formation and improve fruit setting in pears, almonds, vines, olives and tomatoes; and prevent premature fruit drop in pears, apricots and plums. Chlormequat chloride, as well as methods of preparing chlormequat chloride, are known in the art. Its plant growth regulation activity is described, for example, in The Pesticide Manual, Eighteenth Edition, 2016.

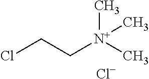

Chlormequat chloride can be applied to vegetation or an area adjacent the vegetation or applied to soil or water to prevent the emergence or growth of vegetation in an amount sufficient to induce a herbicidal effect. In some aspects, chlormequat chloride is applied to vegetation or an area adjacent the vegetation or applied to soil or water to prevent the emergence or growth of vegetation in an amount of 500 g ai/ha or more, such as 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, or 1550 g ai/ha or more; in an amount of 1600 g ai/ha or less, such as 1550 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, or 525 g ai/ha or less; or in an amount ranging from any of the minimum values described above to any of the maximum values described above, such as 500-1600 g ai/ha, 750-1500 g ai/ha, 675-1100 g ai/ha, 600-1300 g ai/ha, 650-1000 g ai/ha, 1100-1400 g ai/ha, 900-1200 g ai/ha, 700-1550 g ai/ha, 500-800 g ai/ha, 800-1200 g ai/ha, or 525-1550 g ai/ha.

### Chlorpropham

Compositions and methods of the present disclosure can include chlorpropham or an agriculturally acceptable salt or ester thereof. Chlorpropham, shown below, is a quaternary ammonium salt used to inhibit sprouting in stored potatoes, at 8-64 g/t and control suckers in tobacco. Chlorpropham is also used for pre-emergence control of annual grass and some broad-leaved weeds in onions, leeks, garlic, shallots, alfalfa, clover, sugar beets, spinach, lettuce, endive, chicory, herbs, peas, beans, carrots, celery, black salsify, fennel, soybeans, cotton, rice, safflowers, sunflowers, cranberries, cane berries, ornamentals, ornamental trees and shrubs, perennial grass seed crops, etc., at up to 240 g/ha. Chlorpropham, as well as methods of preparing chlorpropham, are known in the art. Its plant growth regulation activity is described, for example, in The Pesticide Manual, Eighteenth Edition, 2016.

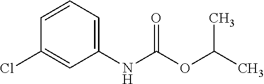

Chlorpropham can be applied to vegetation or an area adjacent the vegetation or applied to soil or water to prevent the emergence or growth of vegetation in an amount sufficient to induce a herbicidal effect. In some aspects, chlorpropham is applied to vegetation or an area adjacent the vegetation or applied to soil or water to prevent the emergence or growth of vegetation in an amount of 15 g ai/ha or more, such as 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, or 245 g ai/ha or more; in an amount of 250 g ai/ha or less, such as 245 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, or 20 g ai/ha or less; or in an amount ranging from any of the minimum values described above to any of the maximum values described above, such as 15-250 g ai/ha, 30-65 g ai/ha, 40-175 g ai/ha, 65-250 g ai/ha, 50-150 g ai/ha, 110-190 g ai/ha, 120-230 g ai/ha, 75-220 g ai/ha, 80-230 g ai/ha, 100-140 g ai/ha, 95-160 g ai/ha, or 20-245 g ai/ha.

### Clofencet

Compositions and methods of the present disclosure can include clofencet or an agriculturally acceptable salt or ester thereof. Clofencet, shown below, is a pyridazinecarboxylic acid used as a chemical hybridizing agent for wheat, at 3000-5000 g/h as the potassium salt. Clofencet, as well as methods of preparing clofencet, are known in the art. Its plant growth regulation activity is described, for example, in The Pesticide Manual, Eighteenth Edition, 2016.

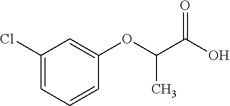

### Cloprop

Compositions and methods of the present disclosure can include cloprop or an agriculturally acceptable salt or ester thereof. Cloprop, shown below, is a phenoxycarboxylic acid used to reduce crown growth in pineapples, in order to increase the fruit size and weight and to delay maturation, and also for thinning of fruit in certain plum varieties. Cloprop, as well as methods of preparing cloprop, are known in the art. Its plant growth regulation activity is described, for example, in The Pesticide Manual, Eighteenth Edition, 2016.

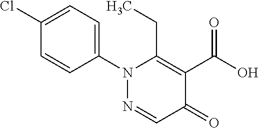 [SMILES: CCc1c(C(=O)O)c(=O)cnn1-c1ccc(Cl)cc1]

Cloprop can be applied to vegetation or an area adjacent the vegetation or applied to soil or water to prevent the emergence or growth of vegetation in an amount sufficient to induce a herbicidal effect. In some aspects, cloprop is applied to vegetation or an area adjacent the vegetation or applied to soil or water to prevent the emergence or growth of vegetation in an amount of 100 g ai/ha or more, such as 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, or 390 g ai/ha or more; in an amount of 400 g ai/ha or less, such as 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, or 110 g ai/ha or less; or in an amount ranging from any of the minimum values described above to any of the maximum values described above, such as 100-400 g ai/ha, 310-380 g ai/ha, 160-375 g ai/ha, 120-250 g ai/ha, 175-350 g ai/ha, 140-240 g ai/ha, 190-370 g ai/ha, 200-220 g ai/ha, 340-400 g ai/ha, 130-375 g ai/ha, 240-380 g ai/ha, or 110-390 g ai/ha.

### Cloxyfonac

Compositions and methods of the present disclosure can include cloxyfonac or an agriculturally acceptable salt or ester thereof. Cloxyfonac, shown below, is a phenoxycarboxylic acid used for fruit setting in tomatoes and aubergines at flowering, producing fruit of uniform size. Cloxyfonac, as well as methods of preparing cloxyfonac, are known in the art. Its plant growth regulation activity is described, for example, in The Pesticide Manual, Eighteenth Edition, 2016.

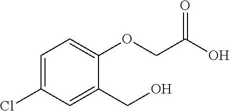 [SMILES: O=C(O)COc1ccc(Cl)cc1CO]

### Cyanamide

Compositions and methods of the present disclosure can include cyanamide or an agriculturally acceptable salt or ester thereof. Cyanamide, shown below, is an inorganic compound used for early post-emergence control of broad-leaved weeds in alliums (onions, leeks, chives, shallots and garlic) and bulb flowers. Cyanamide is also used for promotion of bud break and increase of flowering in kiwifruit and apples, and removal of side shoots and inhibition of sprouting in hops and vines. Cyanamide, as well as methods of preparing cyanamide, are known in the art. Its plant growth regulation activity is described, for example, in The Pesticide Manual, Eighteenth Edition, 2016.

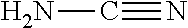

Cyanamide can be applied to vegetation or an area adjacent the vegetation or applied to soil or water to prevent the emergence or growth of vegetation in an amount sufficient to induce a herbicidal effect. In some aspects, cyanamide is applied to vegetation or an area adjacent the vegetation or applied to soil or water to prevent the emergence or growth of vegetation in an amount of 1 kg ai/ha or more, such as 1.1 kg ai/ha or more, 1.25 kg ai/ha or more, 1.5 kg ai/ha or more, 1.75 kg ai/ha or more, 2 kg ai/ha or more, 2.25 kg ai/ha or more, 2.5 kg ai/ha or more, 2.75 kg ai/ha or more, 3 kg ai/ha or more, 3.25 kg ai/ha or more, 3.5 kg ai/ha or more, 3.75 kg ai/ha or more, 4 kg ai/ha or more, 4.25 kg ai/ha or more, 4.5 kg ai/ha or more, or 4.75 kg ai/ha or more; in an amount of 5 kg ai/ha or less, such as 4.75 kg ai/ha or less, 4.5 kg ai/ha or less, 4.25 kg ai/ha or less, 4 kg ai/ha or less, 3.75 kg ai/ha or less, 3.5 kg ai/ha or less, 3.25 kg ai/ha or less, 3 kg ai/ha or less, 2.75 kg ai/ha or less, 2.5 kg ai/ha or less, 2.25 kg ai/ha or less, 2 kg ai/ha or less, 1.75 kg ai/ha or less, 1.5 kg ai/ha or less, 1.25 kg ai/ha or less, or 1.1 kg ai/ha or less; or in an amount ranging from any of the minimum values described above to any of the maximum values described above, such as 1-5 kg ai/ha, 1.25-4 kg ai/ha, 1.1-3.25 kg ai/ha, 2.5-3.75 kg ai/ha, 2-4.25 kg ai/ha, 3.25-4 kg ai/ha, 3-4.5 kg ai/ha, or 1.1-4.75 kg ai/ha.

### Cyclanilide

Compositions and methods of the present disclosure can include cyclanilide or an agriculturally acceptable salt or ester thereof. Cyclanilide, shown below, is cyclopropane carboxylic acid used for promotion of boll opening, defoliation and inhibition of terminal foliar regrowth in cotton. Cyclanilide, as well as methods of preparing cyclanilide, are known in the art. Its plant growth regulation activity is described, for example, in The Pesticide Manual, Eighteenth Edition, 2016.

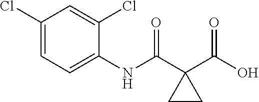 [SMILES: O=C(O)C1(C(=O)Nc2ccc(Cl)cc2Cl)CC1]

Cyclanilide can be applied to vegetation or an area adjacent the vegetation or applied to soil or water to prevent the emergence or growth of vegetation in an amount sufficient to induce a herbicidal effect. In some aspects, cyclanilide is applied to vegetation or an area adjacent the vegetation or applied to soil or water to prevent the emergence or growth of vegetation in an amount of 3 g ai/ha or more, such as 3.25 g ai/ha or more, 3.5 g ai/ha or more, 3.75 g ai/ha or more, 4 g ai/ha or more, 4.25 g ai/ha or more, 4.5 g ai/ha or more, 4.75 g ai/ha or more, 5 g ai/ha or more, 5.25 g ai/ha or more, 5.5 g ai/ha or more, 5.75 g ai/ha or more, 6 g ai/ha or more, 6.25 g ai/ha or more, 6.5 g ai/ha or more, 6.75 g ai/ha or more, 7 g ai/ha or more, 7.25 g ai/ha or more, 7.5 g ai/ha or more, 8 g ai/ha or more, 8.5 g ai/ha or more, 9 g ai/ha or more, 9.5 g ai/ha, or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, or 140 g ai/ha or more; in an amount of 150 g ai/ha or less, such as 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9.5 g ai/ha or less, 9 g ai/ha or less, 8.5 g ai/ha or less, 8 g ai/ha or less 7.5 g ai/ha or less, 7.25 g ai/ha or less, 7 g ai/ha or less, 6.75 g ai/ha or less, 6.5 g ai/ha or less, 6.25 g ai/ha or less, 6 g ai/ha or less, 5.75 g ai/ha or less, 5.5 g ai/ha or less, 5.25 g ai/ha or less, 5 g ai/ha or less, 4.75 g ai/ha or less, 4.5 g ai/ha or less, 4.25 g ai/ha or less, 4 g ai/ha or less, 3.75 g ai/ha or less, 3.5 g ai/ha or less, or 3.25 g ai/ha or less; or in an amount ranging from any of the minimum values described above to any of the maximum values described above, such as 3-150 g ai/ha, 9.5-65 g ai/ha, 40-130 g ai/ha, 6-25 g ai/ha, 50-120 g ai/ha, 110-150 g ai/ha, 15-40 g ai/ha, 75-125 g ai/ha, 30-85 g ai/ha, 100-140 g ai/ha, 4.5-20 g ai/ha, or 3.25-140 g ai/ha.

### Cytokinins

Compositions and methods of the present disclosure can include the cytokinins, such as 6-isopentenylaminopurine, zeatin, and kinetin, or agriculturally acceptable salts or esters thereof. These cytokinins, shown below, are purines that enhance germination, flowering, fruit and seed set, induction of callus initiation, reduction of apical dominance, breaking lateral bud dormancy and retarding of aging in vegetables, peas, beans, tree fruit, vines, ornamentals, flowers, containerized plants, lawn and turf. Cytokinins, as well as methods of preparing

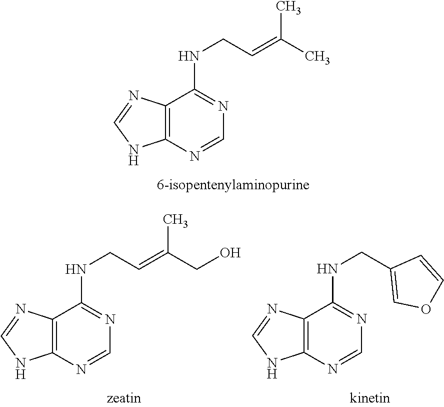 [SMILES: CC(C)=CCNc1ncnc2[nH]cnc12 ; C/C(=C\CNc1ncnc2[nH]cnc12)CO ; c1nc(NCc2ccoc2)c2nc[nH]c2n1]

cytokinins, are known in the art. Their plant growth regulation activity is described, for example, in The Pesticide Manual, Eighteenth Edition, 2016.

Cytokinins can be applied to vegetation or an area adjacent the vegetation or applied to soil or water to prevent the emergence or growth of vegetation in an amount sufficient to induce a herbicidal effect. In some aspects, cytokinins are applied to vegetation or an area adjacent the vegetation or applied to soil or water to prevent the emergence or growth of vegetation in an amount of 0.02 g ai/ha or more, such as 0.025 g ai/ha or more, 0.03 g ai/ha or more, 0.04 g ai/ha or more, 0.05 g ai/ha or more, 0.06 g ai/ha or more, 0.07 g ai/ha or more, 0.08 g ai/ha or more, 0.09 g ai/ha or more, 0.1 g ai/ha or more, 0.11 g ai/ha or more, 0.125 g ai/ha or more, 0.15 g ai/ha or more, 0.175 g ai/ha or more, 0.20 g ai/ha or more, 0.25 g ai/ha or more, 0.3 g ai/ha or more, 0.4 g ai/ha or more, 0.5 g ai/ha or more, 0.6 g ai/ha or more, 0.7 g ai/ha or more, 0.8 g ai/ha or more, 0.9 g ai/ha or more, 1 g ai/ha or more, 1.1 g ai/ha or more, 1.25 g ai/ha or more, 1.5 g ai/ha or more, 1.75 g ai/ha or more, 2 g ai/ha or more, 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.25 g ai/ha or more, 4.5 g ai/ha or more, 4.75 g ai/ha or more, 5 g ai/ha or more, 5.25 g ai/ha or more, 5.5 g ai/ha or more, 5.75 g ai/ha or more, 6 g ai/ha or more, 6.25 g ai/ha or more, 6.5 g ai/ha or more, 6.75 g ai/ha or more, 7 g ai/ha or more, or 7.25 g ai/ha or more; in an amount of 7.5 g ai/ha or less, such as 7.25 g ai/ha or less, 7 g ai/ha or less, 6.75 g ai/ha or less, 6.5 g ai/ha or less, 6.25 g ai/ha or less, 6 g ai/ha or less, 5.75 g ai/ha or less, 5.5 g ai/ha or less, 5.25 g ai/ha or less, 5 g ai/ha or less, 4.75 g ai/ha or less, 4.5 g ai/ha or less, 4.25 g ai/ha or less, 4 g ai/ha or less, 3.75 g ai/ha or less, 3.5 g ai/ha or less, 3.25 g ai/ha or less, 3 g ai/ha or less, 2.75 g ai/ha or less, 2.5 g ai/ha or less, 2.25 g ai/ha or less, 2 g ai/ha or less, 1.75 g ai/ha or less, 1.5 g ai/ha or less, 1.25 g ai/ha or less, 1 g ai/ha or less, 0.9 g ai/ha or less, 0.8 g ai/ha or less, 0.7 g ai/ha or less, 0.6 g ai/ha or less, 0.5 g ai/ha or less, 0.4 g ai/ha or less, 0.3 g ai/ha or less, 0.2 g ai/ha or less, 0.175 g ai/ha or less, 0.15 g ai/ha or less, 0.125 g ai/ha or less, 0.11 g ai/ha or less, 0.1 g ai/ha or less, 0.09 g ai/ha or less, 0.08 g ai/ha or less, 0.07 g ai/ha or less, 0.06 g ai/ha or less, 0.05 g ai/ha or less, 0.04 g ai/ha or less, 0.03 g ai/ha or less, or 0.025 g ai/ha or less; or in an amount ranging from any of the minimum values described above to any of the maximum values described above, such as 0.02-7.0 g ai/ha, 0.25-4 g ai/ha, 0.09-6.25 g ai/ha, 0.5-2.25 g ai/ha, 0.06-0.9 g ai/ha, 0.7-4.75 g ai/ha, 1.5-4.5 g ai/ha, 0.1-2 g ai/ha, 1-6 g ai/ha, 0.15-3.75 g ai/ha, 2-5.5 g ai/ha, 3.25-6.5 g ai/ha, 0.3-2.5 g ai/ha, or 0.025-7.25 g ai/ha.

### Daminozide

Compositions and methods of the present disclosure can include daminozide or an agriculturally acceptable salt or ester thereof. Daminozide, shown below, is succinamic acid used for inhibition of internodal elongation, to produce more compact plants, in chrysanthemums, azaleas, hydrangeas, poinsettias and other ornamentals, at 106-425 g/ha. Daminozide, as well as methods of preparing daminozide, are known in the art. Its plant growth regulation activity is described, for example, in The Pesticide Manual, Eighteenth Edition, 2016.

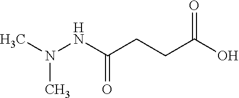

Daminozide can be applied to vegetation or an area adjacent the vegetation or applied to soil or water to prevent the emergence or growth of vegetation in an amount sufficient to induce a herbicidal effect. In some aspects, daminozide is applied to vegetation or an area adjacent the vegetation or applied to soil or water to prevent the emergence or growth of vegetation in an amount of 100 g ai/ha or more, such as 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, or 490 g ai/ha or more; in an amount of 500 g ai/ha or less, such as 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, or 110 g ai/ha or less; or in an amount ranging from any of the minimum values described above to any of the maximum values described above, such as 100-500 g ai/ha, 350-470 g ai/ha, 160-375 g ai/ha, 180-250 g ai/ha, 150-380 g ai/ha, 110-270 g ai/ha, 190-420 g ai/ha, 140-240 g ai/ha, 380-425 g ai/ha, 210-400 g ai/ha, 440-490 g ai/ha, or 110-480 g ai/ha.

### Decan-1-ol

Compositions and methods of the present disclosure can include decan-1-ol or an agriculturally acceptable salt or ester thereof. Decan-1-ol, shown below, is a fatty alcohol used for control of sucker growth in tobacco, at 11000-17000 g/ha. Decan-1-ol, as well as methods of preparing decan-1-ol, are known in the art. Its plant growth regulation activity is described, for example, in The Pesticide Manual, Eighteenth Edition, 2016.

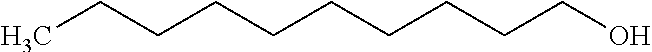

Decan-1-ol can be applied to vegetation or an area adjacent the vegetation or applied to soil or water to prevent the emergence or growth of vegetation in an amount sufficient to induce a herbicidal effect. In some aspects, decan-1-ol is applied to vegetation or an area adjacent the vegetation or applied to soil or water to prevent the emergence or growth of vegetation in an amount of 10 kg ai/ha or more, such as 10.5 kg ai/ha or more, 11 kg ai/ha or more, 11.5 kg ai/ha or more, 12 kg ai/ha or more, 12.5 kg ai/ha or more, 13 kg ai/ha or more, 13.5 kg ai/ha or more, 14 kg ai/ha or more, 14.5 kg ai/ha or more, 15 kg ai/ha or more, 15.5 kg ai/ha or more, 16 kg ai/ha or more, 16.5 kg ai/ha or more, or 16.75 kg ai/ha or more; in an amount of 17 kg ai/ha or less, such as 16.75 kg ai/ha or less, 16.5 kg ai/ha or less, 16 kg ai/ha or less, 15.5 kg ai/ha or less, 15 kg ai/ha or less, 14.5 kg ai/ha or less, 14 kg ai/ha or less, 13.5 kg ai/ha or less, 13 kg ai/ha or less, 12.5 kg ai/ha or less, 12 kg ai/ha or less, 11.5 kg ai/ha or less, 11 kg ai/ha or less, or 10.5 kg ai/ha; or in an amount ranging from any of the minimum values described above to any of the maximum values described above, such as 10-17 kg ai/ha, 11.5-16.5 kg ai/ha, 15.5-17 kg ai/ha, 11-12.5 kg ai/ha, 14-16 kg ai/ha, 11-14.5 kg ai/ha, 12-15 kg ai/ha, 13-13.5 kg ai/ha, 11.5-13 kg ai/ha, or 10.5-16.75 kg ai/ha.

### Dikegulac

Compositions and methods of the present disclosure can include dikegulac or an agriculturally acceptable salt or ester thereof. Dikegulac, shown below, is a plant growth regulator used for reducing apical dominance and promotion of side-branching and flower-bud formation in azaleas, fuchsias and other ornamental plants, and to temporarily retard longitudinal growth in hedges and ornamental shrubs; also for growth retardant in trees by trunk injection. Dikegulac, as well as methods of preparing dikegulac, are known in the art. Its plant growth regulation activity is described, for example, in The Pesticide Manual, Eighteenth Edition, 2016.

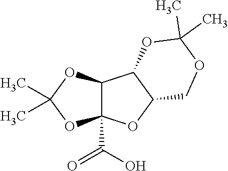

### Dimethipin

Compositions and methods of the present disclosure can include dimethipin or an agriculturally acceptable salt or ester thereof. Dimethipin, shown below, is a dithiine used for: defoliation of cotton, nursery stock, rubber trees and vines, at 260-720 g/ha; destruction of potato haulm, at 480-720 g/ha; and reduction of seed moisture content at harvest in maize, rice, oilseed rape, flax and sunflowers, at 240-720 g/ha. Dimethipin, as well as methods of preparing dimethipin, are known in the art. Its plant growth regulation activity is described, for example, in The Pesticide Manual, Eighteenth Edition, 2016.

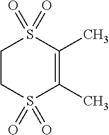

### Dimexano

Compositions and methods of the present disclosure can include dimexano or an agriculturally acceptable salt or ester thereof. Dimexano, shown below, is a thiocarbamate used for: pre-emergence control of broad-leaved weeds in drilled crops, at 9000 g/ha; pre-harvest desiccant for onions and peas, at 28000 g/ha; and control of growth and splitting of carrots. Dimexano, as well as methods of preparing dimexano, are known in the art. Its plant growth regulation activity is described, for example, in The Pesticide Manual, Eighteenth Edition, 2016.

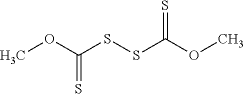

### Endothall

Compositions and methods of the present disclosure can include endothall or an agriculturally acceptable salt or ester thereof. Endothall, shown below, is a bicyclocarboxylic acid used for: pre- and post-emergence control of annual grass and broad-leaved weeds in sugar beets, fodder beets, beetroot, spinach and turf, at 2000-6000 g/ha; control of algae and aquatic weeds at 1-5 ppm; desiccant for alfalfa, clover and hops; defoliant in cotton; and destruction of potato haulms. Endothall, as well as methods of preparing endothall, are known in the art. Its plant growth regulation activity is described, for example, in The Pesticide Manual, Eighteenth Edition, 2016.

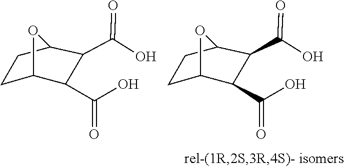

Endothall can be applied to vegetation or an area adjacent the vegetation or applied to soil or water to prevent the emergence or growth of vegetation in an amount sufficient to induce a herbicidal effect. In some aspects, endothall is applied to vegetation or an area adjacent the vegetation or applied to soil or water to prevent the emergence or growth of vegetation in an amount of 600 g ai/ha or more, such as 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3250 g ai/ha or more, 3500 g ai/ha or more, 3750 g ai/ha or more, 4000 g ai/ha or more, 4250 g ai/ha or more, 4500 g ai/ha or more, 4750 g ai/ha or more, 5000 g ai/ha or more, 5250 g ai/ha or more, 5500 g ai/ha or more, or 5750 g ai/ha or more; in an amount of 6000 g ai/ha or less, such as 5750 g ai/ha or less, 5500 g ai/ha or less, 5250 g ai/ha or less, 5000 g ai/ha or less, 4750 g ai/ha or less, 4500 g ai/ha or less, 4250 g ai/ha or less, 4000 g ai/ha or less, 3750 g ai/ha or less, 3500 g ai/ha or less, 3250 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, or 625 g ai/ha or less; or in an amount ranging from any of the minimum values described above to any of the maximum values described above, such as 600-6000 g ai/ha, 950-4500 g ai/ha, 850-3750 g ai/ha, 800-2500 g ai/ha, 3250-5000 g ai/ha, 1100-4750 g ai/ha, 1900-4000 g ai/ha, 2700-5250 g ai/ha, 1800-3250 g ai/ha, 2500-4500 g ai/ha, or 625-5750 g ai/ha.

### Ethephon

Compositions and methods of the present disclosure can include ethephon or an agriculturally acceptable salt or ester thereof. Ethephon, shown below, is a phosphonate used for; promotion of pre-harvest ripening in apples, currants, blackberries, blueberries, cranberries, morello cherries, citrus, figs, tomatoes, sugar beet and fodder beet seed crops, coffee and peppers; acceleration of post-harvest ripening in bananas, mangoes and citrus; loosening of fruit in currants, gooseberries, cherries and apples; increase flower bud development in apples; prevention of lodging in cereals, maize and flax; induction of flowering in Bromeliads; stimulation of lateral branching in azaleas, geraniums and roses; shortening of stem length in forced daffodils; induction of flowering and regulation of ripening in pineapples; accelerate of boll opening in cotton; modification of sex expression in cucumbers and squash; increase fruit setting and yield in cucumbers; improvement of sturdiness in onion seed crops; hasten yellowing in mature tobacco leaves; stimulation of latex flow in rubber trees and resin flow in pine trees; and stimulation of early uniform hull split in walnuts, at 2180 g/ha in cotton, 720 g/ha in cereals and 1440 g/ha in fruit. Ethephon, as well as methods of preparing ethephon, are known in the art. Its plant growth regulation activity is described, for example, in The Pesticide Manual, Eighteenth Edition, 2016.

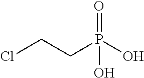

Ethephon can be applied to vegetation or an area adjacent the vegetation or applied to soil or water to prevent the emergence or growth of vegetation in an amount sufficient to induce a herbicidal effect. In some aspects, ethephon is applied to vegetation or an area adjacent the vegetation or applied to soil or water to prevent the emergence or growth of vegetation in an amount of 125 g ai/ha or more, such as 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, or 2400 g ai/ha or more; in an amount of 2500 g ai/ha or less, such as 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, or 130 g ai/ha or less; or in an amount ranging from any of the minimum values described above to any of the maximum values described above, such as 125-2500 g ai/ha, 950-2000 g ai/ha, 1200-2200 g ai/ha, 350-1500 g ai/ha, 1150-2100 g ai/ha, 700-1400 g ai/ha, 260-1100 g ai/ha, 1050-1800 g ai/ha, 1600-2500 g ai/ha, 175-400 g ai/ha, 900-1900 g ai/ha, or 150-2400 g ai/ha.

### Fenoprop

Compositions and methods of the present disclosure can include fenoprop or an agriculturally acceptable salt or ester thereof. Fenoprop, shown below, is a phenoxycarboxylic acid used for postemergence brush control, at 2000-4000 g/hl spray solution; control of aquatic weeds; control of broad-leaved weeds in maize and sugar cane; and reduction of pre-harvest apple drop, at 1400 g ae/hl of amine salt spray solution 7-14 d prior to harvest. Fenoprop, as well as methods of preparing fenoprop, are known in the art. Its plant growth regulation activity is described, for example, in The Pesticide Manual, Eighteenth Edition, 2016.

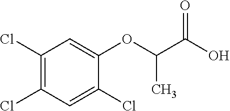

### Flumetralin

Compositions and methods of the present disclosure can include flumetralin or an agriculturally acceptable salt or ester thereof. Flumetralin, shown below, is a dinitroaniline used for control of sucker growth in tobacco. Flumetralin, as well as methods of preparing flumetralin, are known in the art. Its plant growth regulation activity is described, for example, in The Pesticide Manual, Eighteenth Edition, 2016.

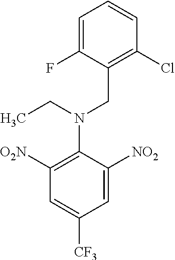 [SMILES: CCN(Cc1c(F)cccc1Cl)c1c([N+](=O)[O-])cc(C(F)(F)F)cc1[N+](=O)[O-]]

Flumetralin can be applied to vegetation or an area adjacent the vegetation or applied to soil or water to prevent the emergence or growth of vegetation in an amount sufficient to induce a herbicidal effect. In some aspects, flumetralin is applied to vegetation or an area adjacent the vegetation or applied to soil or water to prevent the emergence or growth of vegetation in an amount of 650 g ai/ha or more, such as 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, or 1300 g ai/ha or more; in an amount of 1350 g ai/ha or less, such as 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, or 675 g ai/ha or less; or in an amount ranging from any of the minimum values described above to any of the maximum values described above, such as 650-1350 g ai/ha, 850-1100 g ai/ha, 1200-1300 g ai/ha, 700-1050 g ai/ha, 750-1000 g ai/ha, 1100-1150 g ai/ha, 900-1250 g ai/ha, 650-1200 g ai/ha, 800-1000 g ai/ha, 1050-1300 g ai/ha, 700-1200 g ai/ha, or 675-1300 g ai/ha.

### Flurenol

Compositions and methods of the present disclosure can include flurenol or an agriculturally acceptable salt or ester thereof. Flurenol, shown below, is a carboxylic acid used for post-emergence control of broad-leaved weeds in cereals, including wheat, barley, rye, oats, and rice. Flurenol, as well as methods of preparing flurenol, are known in the art. Its plant growth regulation activity is described, for example, in The Pesticide Manual, Eighteenth

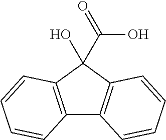 [SMILES: O=C(O)C1(O)c2ccccc2-c2ccccc21]

### Edition, 2016.

Flurenol can be applied to vegetation or an area adjacent the vegetation or applied to soil or water to prevent the emergence or growth of vegetation in an amount sufficient to induce a herbicidal effect. In some aspects, flurenol is applied to vegetation or an area adjacent the vegetation or applied to soil or water to prevent the emergence or growth of vegetation in an amount of 200 g ai/ha or more, such 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, or 950 g ai/ha or more; in an amount of 1000 g ai/ha or less, such as 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, or 210 g ai/ha or less; or in an amount ranging from any of the minimum values described above to any of the maximum values described above, such as 200-1000 g ai/ha, 250-650 g ai/ha, 460-750 g ai/ha, 600-900 g ai/ha, 380-500 g ai/ha, 210-450 g ai/ha, 390-480 g ai/ha, 750-950 g ai/ha, 325-525 g ai/ha, 500-750 g ai/ha, 440-800 g ai/ha, or 210-950 g ai/ha.

### Flurprimidol

Compositions and methods of the present disclosure can include flurprimidol or an agriculturally acceptable salt or ester thereof. Flurprimidol, shown below, is a pyrimidine carbinol used to decrease the rate of growth of plants, including perennial turf grasses, ornamental cover species, herbaceous and woody ornamentals and deciduous and coniferous trees, at 140-1100 g/ha in turf grasses, 560-1100 g/ha in flowering and foliage plants, 1100-3400 g/ha in woody ornamentals. Flurprimidol, as well as methods of preparing flurprimidol,

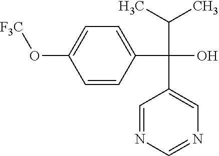 [SMILES: CC(C)C(O)(c1ccc(OC(F)(F)F)cc1)c1cncnc1]

are known in the art. Its plant growth regulation activity is described, for example, in The Pesticide Manual, Eighteenth Edition, 2016.

Flurprimidol can be applied to vegetation or an area adjacent the vegetation or applied to soil or water to prevent the emergence or growth of vegetation in an amount sufficient to induce a herbicidal effect. In some aspects, flurprimidol is applied to vegetation or an area adjacent the vegetation or applied to soil or water to prevent the emergence or growth of vegetation in an amount of 125 g ai/ha or more, such as 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, or 3400 g ai/ha or more; in an amount of 3500 g ai/ha or less, such as 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, or 130 g ai/ha or less; or in an amount ranging from any of the minimum values described above to any of the maximum values described above, such as 125-3500 g ai/ha, 950-2500 g ai/ha, 460-1700 g ai/ha, 1600-2500 g ai/ha, 550-1800 g ai/ha, 210-470 g ai/ha, 1300-3300 g ai/ha, 340-2200 g ai/ha, 650-3250 g ai/ha, 1000-1400 g ai/ha, 440-2900 g ai/ha, or 130-3400 g ai/ha.

### Forchlorfenuron

Compositions and methods of the present disclosure can include forchlorfenuron or an agriculturally acceptable salt or ester thereof. Forchlorfenuron, shown below, is a phenyl urea used for: increasing the size of kiwifruit, table grapes and peaches; promotion of fruit set in melons, pumpkins and cucumbers; promotion of branching in apples; increasing yields in potatoes, rice and wheat. Forchlorfenuron, as well as methods of preparing forchlorfenuron, are known in the art. Its plant growth regulation activity is described, for example, in The Pesticide Manual, Eighteenth Edition, 2016.

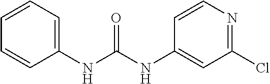 [SMILES: O=C(Nc1ccccc1)Nc1ccnc(Cl)c1]

Forchlorfenuron can be applied to vegetation or an area adjacent the vegetation or applied to soil or water to prevent the emergence or growth of vegetation in an amount sufficient to induce a herbicidal effect. In some aspects, forchlorfenuron is applied to vegetation or an area adjacent the vegetation or applied to soil or water to prevent the emergence or growth of vegetation in an amount of 4 g ai/ha or more, such as 4.25 g ai/ha or more, 4.5 g ai/ha or more, 4.75 g ai/ha or more, 5 g ai/ha or more, 5.25 g ai/ha or more, 5.5 g ai/ha or more, 5.75 g ai/ha or more, 6 g ai/ha or more, 6.25 g ai/ha or more, 6.5 g ai/ha or more, 6.75 g ai/ha or more, 7 g ai/ha or more, 7.25 g ai/ha or more, 7.5 g ai/ha or more, 8 g ai/ha or more, 8.5 g ai/ha or more, 9 g ai/ha or more, 9.5 g ai/ha, or more, 10 g ai/ha or more, 12.5 g ai/ha or more, 15 g ai/ha or more, 17.5 g ai/ha or more, 20 g ai/ha or more, 22.5 g ai/ha or more, 25 g ai/ha or more, or 27.5 g ai/ha or more; in an amount of 30 g ai/ha or less, such as 27.5 g ai/ha or less, 25 g ai/ha or less, 22.5 g ai/ha or less, 20 g ai/ha or less, 17.5 g ai/ha or less, 15 g ai/ha or less, 12.5 g ai/ha or less, 10 g ai/ha or less, 9.5 g ai/ha or less, 9 g ai/ha or less, 8.5 g ai/ha or less, 8 g ai/ha or less, 7.5 g ai/ha or less, 7.25 g ai/ha or less, 7 g ai/ha or less, 6.75 g ai/ha or less, 6.5 g ai/ha or less, 6.25 g ai/ha or less, 6 g ai/ha or less, 5.75 g ai/ha or less, 5.5 g ai/ha or less, 5.25 g ai/ha or less, 5 g ai/ha or less, 4.75 g ai/ha or less, 4.5 g ai/ha or less, or 4.25 g ai/ha or less; or in an amount ranging from any of the minimum values described above to any of the maximum values described above, such as 4-30 g ai/ha, 5.25-7.5 g ai/ha, 12-27.5 g ai/ha, 8.5-20 g ai/ha, 6.25-9.5 g ai/ha, 17.5-27.5 g ai/ha, 8-25 g ai/ha, 10-20 g ai/ha, 4.25-7.5 g ai/ha, 9.5-15 g ai/ha, 7-15 g ai/ha, 4-8.5 g ai/ha, or 4.25-27.5 g ai/ha.

### Gibberellins

Compositions and methods of the present disclosure can include gibberellins, including gibberellic acid, gibberellin A4, and gibberellin A7, or an agriculturally acceptable salt or ester thereof. Gibberellins, shown below, are natural plant compounds used to: improve fruit setting in clementines and pears; loosen and elongate clusters and increase berry size in grapes; control fruit maturity by delaying development of the yellow color in lemons; reduce rind stain and retard rind ageing in navel oranges; promote uniform seedling growth in rice; promote elongation in winter celery; induce uniform bolting and increase seed production in lettuce; stimulate sprouting in seed potatoes; hasten maturation in artichokes; increase yields in forced rhubarb; increase malting quality in barley; produce brighter-colored, firmer fruit and increase fruit size in sweet cherries; increase yields and aid harvesting in hops; reduce internal browning and increase yields in Italian prunes; increase fruit set and yields in tangelos and tangerines; improve fruit setting in blueberries; promote flowering and increase yield in strawberries, all at up to 200 g/ha. Gibberellins, as well as methods of preparing gibberellins, are known in the art. Their plant growth regulation activity is described, for example, in The Pesticide Manual, Eighteenth Edition, 2016.

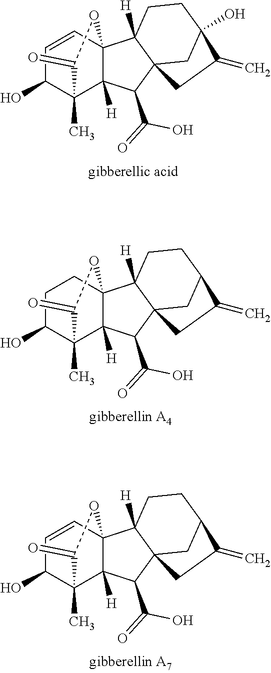

Gibberellins can be applied to vegetation or an area adjacent the vegetation or applied to soil or water to prevent the emergence or growth of vegetation in an amount sufficient to induce a herbicidal effect. In some aspects, gibberellins are applied to vegetation or an area adjacent the vegetation or applied to soil or water to prevent the emergence or growth of vegetation in an amount of 1 g ai/ha or more, such as 1.1 g ai/ha or more, 1.25 g ai/ha or more, 1.5 g ai/ha or more, 1.75 g ai/ha or more, 2 g ai/ha or more, 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.25 g ai/ha or more, 4.5 g ai/ha or more, 4.75 g ai/ha or more, 5 g ai/ha or more, 5.25 g ai/ha or more, 5.5 g ai/ha or more, 5.75 g ai/ha or more, 6 g ai/ha or more, 6.25 g ai/ha or more, 6.5 g ai/ha or more, 6.75 g ai/ha or more, 7 g ai/ha or more, 7.25 g ai/ha or more, 7.5 g ai/ha or more, 8 g ai/ha or more, 8.5 g ai/ha or more, 9 g ai/ha or more, 9.5 g ai/ha, or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, or 340 g ai/ha or more; in an amount of 350 g ai/ha or less, such as 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9.5 g ai/ha or less, 9 g ai/ha or less, 8.5 g ai/ha or less, 8 g ai/ha or less, 7.5 g ai/ha or less, 7.25 g ai/ha or less, 7 g ai/ha or less, 6.75 g ai/ha or less, 6.5 g ai/ha or less, 6.25 g ai/ha or less, 6 g ai/ha or less, 5.75 g ai/ha or less, 5.5 g ai/ha or less, 5.25 g ai/ha or less, 5 g ai/ha or less, 4.75 g ai/ha or less, 4.5 g ai/ha or less, 4.25 g ai/ha or less, 4 g ai/ha or less, 3.75 g ai/ha or less, 3.5 g ai/ha or less, 3.25 g ai/ha or less, 3 g ai/ha or less, 2.75 g ai/ha or less, 2.5 g ai/ha or less, 2.25 g ai/ha or less, 2 g ai/ha or less, 1.75 g ai/ha or less, 1.5 g ai/ha or less, 1.25 g ai/ha or less, or 1.1 g ai/ha or less; or in an amount ranging from any of the minimum values described above to any of the maximum values described above, such as 1-350 g ai/ha, 9.5-70 g ai/ha, 4.5-150 g ai/ha, 60-220 g ai/ha, 5-130 g ai/ha, 10-250 g ai/ha, 25-140 g ai/ha, 75-300 g ai/ha, 8-55 g ai/ha, 30-160 g ai/ha, 110-290 g ai/ha, or 1.1-340 g ai/ha.

### Heptamaloxyloglucan

Compositions and methods of the present disclosure can include heptamaloxyloglucan or an agriculturally acceptable salt or ester thereof. Heptamaloxyloglucan, shown below, is a polysaccharide used as a plant elicitor for protection of grapes from spring frost damage. Heptamaloxyloglucan, as well as methods of preparing heptamaloxyloglucan, are known in the art. Its plant growth regulation activity is described, for example, in The Pesticide Manual, Eighteenth Edition, 2016.

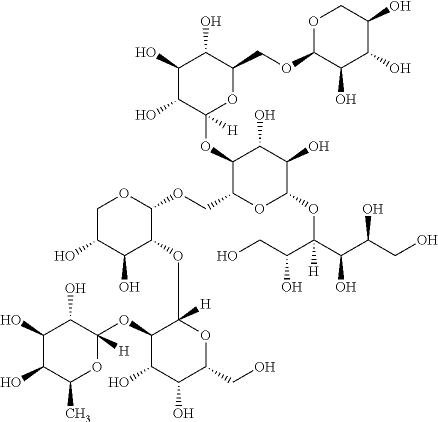 [SMILES: C[C@@H]1O[C@@H](O[C@H]2[C@H](O[C@H]3[C@@H](OC[C@H]4O[C@@H](O[C@@H]([C@H](O)[C@@H](O)CO)[C@H](O)CO)[C@H](O)[C@@H](O)[C@@H]4O[C@@H]4O[C@H](CO[C@H]5OC[C@@H](O)[C@H](O)[C@H]5O)[C@@H](O)[C@H](O)[C@H]4O)OC[C@@H](O)[C@@H]3O)O[C@H](CO)[C@H](O)[C@@H]2O)[C@@H](O)[C@H](O)[C@@H]1O]

### Indol-3-Butyric Acid (IBA) and Indol-3-Ylacetic Acid (IAA)

Compositions and methods of the present disclosure can include indol-3-butyric acid (IBA), indol-3-ylacetic acid (IAA) or an agriculturally acceptable salt or ester thereof. IBA and IAA, shown below, are plant hormones used to stimulate rooting of cuttings of herbaceous and woody ornamentals. IBA and IAA, as well as methods of preparing IBA and IAA, are known in the art. Their plant growth regulation activity is described, for example, in The Pesticide Manual, Eighteenth Edition, 2016.

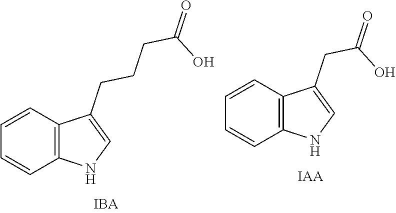

IBA or IAA can be applied to vegetation or an area adjacent the vegetation or applied to soil or water to prevent the emergence or growth of vegetation in an amount sufficient to induce a herbicidal effect. In some aspects, IBA or IAA are applied to vegetation or an area adjacent the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 105 g ai/ha or more, 110 g ai/ha or more, 115 g ai/ha or more, 120 g ai/ha or more, 125 g ai/ha or more, 130 g ai/ha or more, 135 g ai/ha or more, 140 g ai/ha or more, or 145 g ai/ha or more; in an amount of 150 g ai/ha or less, 145 g ai/ha or less, 140 g ai/ha or less, 135 g ai/ha or less, 130 g ai/ha or less, 125 g ai/ha or less, 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-150 g ai/ha, 95-130 g ai/ha, 60-140 g ai/ha, 70-125 g ai/ha, 100-150 g ai/ha, 55-115 g ai/ha, 75-100 g ai/ha, 85-120 g ai/ha, 90-125 g ai/ha, or 55-145 g ai/ha.

### Isoprothiolane

Compositions and methods of the present disclosure can include isoprothiolane or an agriculturally acceptable salt or ester thereof. Isoprothiolane, shown below, is a dithiolane used to accelerate rooting, promote root elongation, and control non-parasitic damping-off in rice. Isoprothiolane, as well as methods of preparing isoprothiolane, are known in the art. Its plant growth regulation activity is described, for example, in The Pesticide Manual, Eighteenth Edition, 2016.

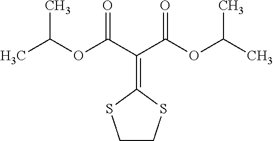 [SMILES: CC(C)OC(=O)C(C(=O)OC(C)C)=C1SCCS1]

### Maleic Hydrazide

Compositions and methods of the present disclosure can include maleic hydrazide or an agriculturally acceptable salt or ester thereof. Maleic hydrazide, shown below, is a pyridazine used for: suppression of grass growth in lawns, roadside verges, embankments and amenity areas and of growth of shrubs and trees; inhibition of sprouting in potatoes, onions, beets, swedes, parsnips and carrots in storage; prevention of sucker development in tobacco; induction of dormancy in citrus, at 1200-5400 g/ha. Maleic hydrazide, as well as methods of preparing maleic hydrazide, are known in the art. Its plant growth regulation activity is described, for example, in The Pesticide Manual, Eighteenth Edition, 2016.

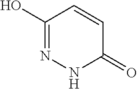

Maleic hydrazide can be applied to vegetation or an area adjacent the vegetation or applied to soil or water to prevent the emergence or growth of vegetation in an amount sufficient to induce a herbicidal effect. In some aspects, maleic hydrazide is applied to vegetation or an area adjacent the vegetation or applied to soil or water to prevent the emergence or growth of vegetation in an amount of 1000 g ai/ha or more, such as 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3750 g ai/ha or more, 4000 g ai/ha or more, 4250 g ai/ha or more, 4500 g ai/ha or more, 4750 g ai/ha or more, 5000 g ai/ha or more, 5250 g ai/ha or more; in an amount of 5500 g ai/ha or less, such as 5250 g ai/ha or less, 5000 g ai/ha or less, 4750 g ai/ha or less, 4500 g ai/ha or less, 4250 g ai/ha or less, 4000 g ai/ha or less, 3750 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, or 1050 g ai/ha or less; or in an amount ranging from any of the minimum values described above to any of the maximum values described above, such as 1000-5500 g ai/ha, 1250-4500 g ai/ha, 4500-5250 g ai/ha, 1600-2500 g ai/ha, 1150-3100 g ai/ha, 1350-1700 g ai/ha, 1900-3000 g ai/ha, 2400-4250 g ai/ha, 3200-5000 g ai/ha, 1400-4000 g ai/ha, 1800-2800 g ai/ha, or 1050-5250 g ai/ha.

### Mefluidide

Compositions and methods of the present disclosure can include mefluidide or an agriculturally acceptable salt or ester thereof. Mefluidide, shown below, is a sulfonanilide used for: inhibition of growth and suppression of seed production of perennial grasses in turf, lawns, grassland, industrial areas, amenity areas, roadside verges and embankments; inhibition of growth of ornamental trees and shrubs; enhancement of the sucrose content of sugar cane, at 300 to 1100 g/ha; and control of growth and seed production of weeds, particularly Sorghum halepense and volunteer cereals, in soybeans and other crops. Mefluidide, as well as methods of preparing mefluidide, are known in the art. Its plant growth regulation activity is described, for example, in The Pesticide Manual, Eighteenth Edition, 2016.

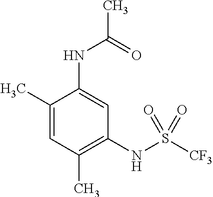 [SMILES: CC(=O)Nc1cc(NS(=O)(=O)C(F)(F)F)c(C)cc1C]

Mefluidide can be applied to vegetation or an area adjacent the vegetation or applied to soil or water to prevent the emergence or growth of vegetation in an amount sufficient to induce a herbicidal effect. In some aspects, mefluidide is applied to vegetation or an area adjacent the vegetation or applied to soil or water to prevent the emergence or growth of vegetation in an amount of 200 g ai/ha or more, such as 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, or 1200 g ai/ha or more; in an amount of 1250 g ai/ha or less, such as 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, or 210 g ai/ha or less; or in an amount ranging from any of the minimum values described above to any of the maximum values described above, such as 200-1250 g ai/ha, 250-650 g ai/ha, 460-950 g ai/ha, 300-700 g ai/ha, 370-800 g ai/ha, 210-575 g ai/ha, 290-400 g ai/ha, 750-1050 g ai/ha, 230-525 g ai/ha, 425-1100 g ai/ha, 340-850 g ai/ha, or 210-1200 g ai/ha.

### Mepiquat

Compositions and methods of the present disclosure can include the mepiquat ion or an agriculturally acceptable salt thereof. Mepiquat, shown below as the chloride or pentaborate salt, is a quaternary ammonium ion used for: reduction of vegetative growth and promotion of boll maturation in cotton, at up to 75 g/ha; inhibition of sprouting in onions, garlic and leeks; prevention of lodging, by shortening the stem and strengthening the stem wall, in cereals, grass seed crops and flax, at up to 762.5 g/ha. Mepiquat, as well as methods of preparing mepiquat, are known in the art. Its plant growth regulation activity is described, for example, in The Pesticide Manual, Eighteenth Edition, 2016.

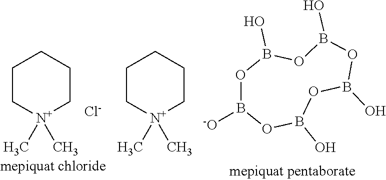

Mepiquat can be applied to vegetation or an area adjacent the vegetation or applied to soil or water to prevent the emergence or growth of vegetation in an amount sufficient to induce a herbicidal effect. In some aspects, mepiquat is applied to vegetation or an area adjacent the vegetation or applied to soil or water to prevent the emergence or growth of vegetation in an amount of 5 g ai/ha or more, such as 6 g ai/ha or more, 7 g ai/ha or more, 7.5 g ai/ha or more, 8 g ai/ha or more, 9 g ai/ha or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, or 750 g ai/ha or more; in an amount of 800 g ai/ha or less, such as 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9 g ai/ha or less, 8 g ai/ha or less, 7.5 g ai/ha or less, 7 g ai/ha or less, or 6 g ai/ha or less; or in an amount ranging from any of the minimum values described above to any of the maximum values described above, such as 5-800 g ai/ha, 9-65 g ai/ha, 40-375 g ai/ha, 120-250 g ai/ha, 50-550 g ai/ha, 110-450 g ai/ha, 230-400 g ai/ha, 700-800 g ai/ha, 380-525 g ai/ha, 25-140 g ai/ha, 340-675 g ai/ha, or 6-750 g ai/ha.

### N-Acetylthiazolidine-4-Carboxylic Acid

Compositions and methods of the present disclosure can include N-acetylthiazolidine-4-carboxylic acid or an agriculturally acceptable salt or ester thereof. N-Acetylthiazolidine-4-carboxylic acid, shown below, is a carboxylic acid used to increase size of fruit, berries and kernels, increase yield, increase vitamins and improve skin color, using a spray solution at 20-75 ppm concentration. N-Acetylthiazolidine-4-carboxylic acid, as well as

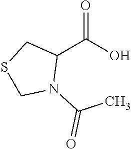

methods of preparing N-acetylthiazolidine-4-carboxylic acid, are known in the art. Its plant growth regulation activity is described, for example, in The Pesticide Manual, Eighteenth Edition, 2016.

### Naphthaleneacetamide

Compositions and methods of the present disclosure can include naphthaleneacetamide or an agriculturally acceptable salt or ester thereof. Naphthaleneacetamide, shown below, is an aromatic amide used to: stimulate root production at 0.1-0.6 g/plant; thin fruit in apples and pears; and prevent premature fruit fall in apples and cherries. Naphthaleneacetamide, as well as methods of preparing naphthaleneacetamide, are known in the art. Its plant growth regulation activity is described, for example, in The Pesticide Manual, Eighteenth Edition, 2016.

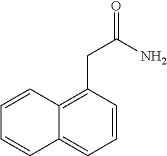 [SMILES: NC(=O)Cc1cccc2ccccc12]

### Nitrophenolates

Compositions and methods of the present disclosure can include the nitrophenolate mixture (sodium 5-nitroguaiacolate, sodium o-nitrophenolate, and sodium p-nitrophenolate) or an agriculturally acceptable salt or ester thereof. Nitrophenolates, shown below, are a mixture of phenolate salts used to increase nutrient uptake in cotton, cereals, cucumbers, grapes, melons, paprika, rice, soybeans, sugar beets and vegetables, at 3-6 g/ha. Nitrophenolates, as well as methods of preparing nitrophenolates, are known in the art. Its plant growth regulation activity is described, for example, in The Pesticide Manual, Eighteenth Edition, 2016.

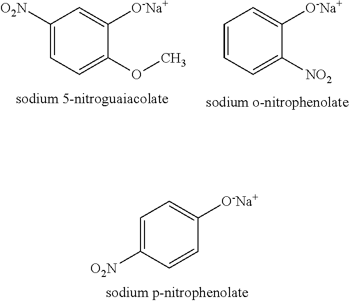

Nitrophenolates can be applied to vegetation or an area adjacent the vegetation or applied to soil or water to prevent the emergence or growth of vegetation in an amount sufficient to induce a herbicidal effect. In some aspects, nitrophenolates are applied to vegetation or an area adjacent the vegetation or applied to soil or water to prevent the emergence or growth of vegetation in an amount of 1 g ai/ha or more, such as 1.1 g ai/ha or more, 1.25 g ai/ha or more, 1.5 g ai/ha or more, 1.75 g ai/ha or more, 2 g ai/ha or more, 2.25 g ai/ha or more, 2.5 g ai/ha or more, 2.75 g ai/ha or more, 3 g ai/ha or more, 3.25 g ai/ha or more, 3.5 g ai/ha or more, 3.75 g ai/ha or more, 4 g ai/ha or more, 4.25 g ai/ha or more, 4.5 g ai/ha or more, 4.75 g ai/ha or more, 5 g ai/ha or more, 5.25 g ai/ha or more, 5.5 g ai/ha or more, 5.75 g ai/ha or more, 6 g ai/ha or more, 6.25 g ai/ha or more, 6.5 g ai/ha or more, 6.75 g ai/ha or more, 7 g ai/ha or more, 7.25 g ai/ha or more, 7.5 g ai/ha or more, 8 g ai/ha or more, 8.5 g ai/ha or more, 9 g ai/ha or more, or 9.5 g ai/ha, or more; in an amount of 10 g ai/ha or less, 9.5 g ai/ha or less, 9 g ai/ha or less, 8.5 g ai/ha or less, 8 g ai/ha or less, 7.5 g ai/ha or less, 7.25 g ai/ha or less, 7 g ai/ha or less, 6.75 g ai/ha or less, 6.5 g ai/ha or less, 6.25 g ai/ha or less, 6 g ai/ha or less, 5.75 g ai/ha or less, 5.5 g ai/ha or less, 5.25 g ai/ha or less, 5 g ai/ha or less, 4.75 g ai/ha or less, 4.5 g ai/ha or less, 4.25 g ai/ha or less, 4 g ai/ha or less, 3.75 g ai/ha or less, 3.5 g ai/ha or less, 3.25 g ai/ha or less, 3 g ai/ha or less, 2.75 g ai/ha or less, 2.5 g ai/ha or less, 2.25 g ai/ha or less, 2 g ai/ha or less, 1.75 g ai/ha or less, 1.5 g ai/ha or less, 1.25 g ai/ha or less, or 1.1 g ai/ha or less; or in an amount ranging from any of the minimum values described above to any of the maximum values described above, such as 1-10 g ai/ha, 2.25-7 g ai/ha, 4.5-6.5 g ai/ha, 1.75-6 g ai/ha, 3-9 g ai/ha, 5.25-8.5 g ai/ha, 1.5-4.5 g ai/ha, 1-4 g ai/ha, 1.75-7.25 g ai/ha, 1.5-5.75 g ai/ha, 2-3.75 g ai/ha, 2.25-10 g ai/ha, 3-8.5 g ai/ha, or 1.1-9.5 g ai/ha.

### Paclobutrazol

Compositions and methods of the present disclosure can include paclobutrazol or an agriculturally acceptable salt or ester thereof. Paclobutrazol, shown below, is triazole used for: inhibition of vegetative growth and enhancement of fruit set in tree fruit; inhibition of growth in pot-grown ornamentals and flowers, e.g. chrysanthemums, begonias, freesias, poinsettias and bulbs, as a spray at a concentration of 1-200 ppm; prevent lodging and increase yield in rice; growth retardation in turf and reduction of height and prevention of lodging in grass seed crops and oilseed rape. Paclobutrazol, as well as methods of preparing paclobutrazol, are known in the art. Its plant growth regulation activity is described, for example, in The Pesticide Manual, Eighteenth Edition, 2016.

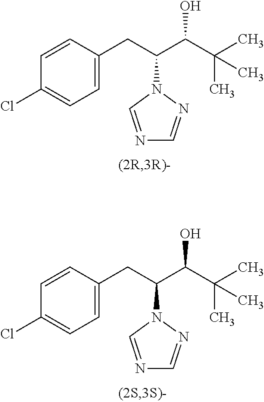 [SMILES: CC(C)(C)[C@@H](O)[C@@H](Cc1ccc(Cl)cc1)n1cncn1 ; CC(C)(C)[C@H](O)[C@H](Cc1ccc(Cl)cc1)n1cncn1]

Paclobutrazol can be applied to vegetation or an area adjacent the vegetation or applied to soil or water to prevent the emergence or growth of vegetation in an amount sufficient to induce a herbicidal effect. In some aspects, paclobutrazol is applied to vegetation or an area adjacent the vegetation or applied to soil or water to prevent the emergence or growth of vegetation in an amount of 40 g ai/ha or more, such as 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, or 950 g ai/ha or more; in an amount of 1000 g ai/ha or less, such as 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, or 45 g ai/ha or less; or in an amount ranging from any of the minimum values described above to any of the maximum values described above, such as 40-1000 g ai/ha, 95-650 g ai/ha, 460-750 g ai/ha, 80-250 g ai/ha, 50-850 g ai/ha, 110-450 g ai/ha, 230-400 g ai/ha, 750-1000 g ai/ha, 380-525 g ai/ha, 320-600 g ai/ha, 65-290 g ai/ha, or 45-950 g ai/ha.

### Pelargonic Acid

Compositions and methods of the present disclosure can include pelargonic acid or an agriculturally acceptable salt or ester thereof. Pelargonic acid, shown below, is fatty acid used for: thinning of blossoms in apples and pears; control of annual weeds in potatoes and peanuts; total weed control in non-crop areas; control of mosses in lawns and other domestic areas. Pelargonic acid, as well as methods of preparing pelargonic acid, are known in the art. Its plant growth regulation activity is described, for example, in The Pesticide Manual, Eighteenth Edition, 2016.

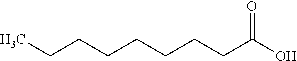

Pelargonic acid can be applied to vegetation or an area adjacent the vegetation or applied to soil or water to prevent the emergence or growth of vegetation in an amount sufficient to induce a herbicidal effect. In some aspects, pelargonic acid is applied to vegetation or an area adjacent the vegetation or applied to soil or water to prevent the emergence or growth of vegetation in an amount of 750 g ai/ha or more, such as 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3750 g ai/ha or more, 4000 g ai/ha or more, 4250 g ai/ha or more, 4500 g ai/ha or more, 4750 g ai/ha or more, 5000 g ai/ha or more, 5250 g ai/ha or more, 5500 g ai/ha or more, 5750 g ai/ha or more, 6000 g ai/ha or more, 6250 g ai/ha or more, 6500 g ai/ha or more, 6750 g ai/ha or more, 7000 g ai/ha or more, 7250 g ai/ha or more, 7500 g ai/ha or more, 7750 g ai/ha or more, 8000 g ai/ha or more, 8250 g ai/ha or more, 8500 g ai/ha or more, 8750 g ai/ha or more, 9000 g ai/ha or more, 9250 g ai/ha or more, 9500 g ai/ha or more, 9750 g ai/ha or more, 10 kg ai/ha or more, 10.5 kg ai/ha or more, 11 kg ai/ha or more, 11.5 kg ai/ha or more, 12 kg ai/ha or more, 12.5 kg ai/ha or more, 13 kg ai/ha or more, 13.5 kg ai/ha or more, 14 kg ai/ha or more, or 14.5 kg ai/ha or more; in an amount of 15 kg ai/ha or less, such as 14.5 kg ai/ha or less, 14 kg ai/ha or less, 13.5 kg ai/ha or less, 13 kg ai/ha or less, 12.5 kg ai/ha or less, 12 kg ai/ha or less, 11.5 kg ai/ha or less, 11 kg ai/ha or less, 10.5 kg ai/ha, 10 kg ai/ha or less, 9750 g ai/ha or less, 9500 g ai/ha or less, 9250 g ai/ha or less, 9000 g ai/ha or less, 8750 g ai/ha or less, 8500 g ai/ha or less, 8250 g ai/ha or less, 8000 g ai/ha or less, 7750 g ai/ha or less, 7500 g ai/ha or less, 7250 g ai/ha or less, 7000 g ai/ha or less, 6750 g ai/ha or less, 6500 g ai/ha or less, 6250 g ai/ha or less, 6000 g ai/ha or less, 5750 g ai/ha or less, 5500 g ai/ha or less, 5250 g ai/ha or less, 5000 g ai/ha or less, 4750 g ai/ha or less, 4500 g ai/ha or less, 4250 g ai/ha or less, 4000 g ai/ha or less, 3750 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, or 800 g ai/ha or less; or in an amount ranging from any of the minimum values described above to any of the maximum values described above, such as 750 g-15 kg ai/ha, 950-6500 g ai/ha, 4000-9750 g ai/ha, 10-14 kg ai/ha, 3500 g-12.5 kg ai/ha, 11-12.5 kg ai/ha, 9500 g-15 kg ai/ha, 4750-8750 g ai/ha, 6500-9250 g ai/ha, 5000 g-14 kg ai/ha, 10.5-13.5 kg ai/ha, or 800 g-14.5 kg ai/ha.

### Prohexadione

Compositions and methods of the present disclosure can include prohexadione or an agriculturally acceptable salt or ester thereof, particularly the calcium salt. Prohexadione, shown below, is fatty acid used for: control of lodging in small-grain cereals, at 75-100 g/ha; growth retardant in turf, at 200-400 g/ha, peanuts, at 100-300 g/ha, and flowers, at 50-100 g/ha; inhibition of new twig elongation in tree fruit, at 200-400 g/ha. Prohexadione, as well as methods of preparing prohexadione, are known in the art. Its plant growth regulation activity is described, for example, in The Pesticide Manual, Eighteenth Edition, 2016.

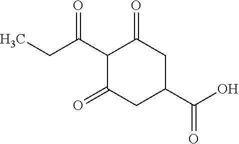

Prohexadione can be applied to vegetation or an area adjacent the vegetation or applied to soil or water to prevent the emergence or growth of vegetation in an amount sufficient to induce a herbicidal effect. In some aspects, prohexadione is applied to vegetation or an area adjacent the vegetation or applied to soil or water to prevent the emergence or growth of vegetation in an amount of 15 g ai/ha or more, such as 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, or 675 g ai/ha or more; in an amount of 700 g ai/ha or less, such as 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, or 20 g ai/ha or less; or in an amount ranging from any of the minimum values described above to any of the maximum values described above, such as 15-700 g ai/ha, 45-650 g ai/ha, 30-375 g ai/ha, 160-250 g ai/ha, 220-550 g ai/ha, 110-450 g ai/ha, 290-490 g ai/ha, 70-430 g ai/ha, 300-525 g ai/ha, 500-650 g ai/ha, 340-575 g ai/ha, or 20-675 g ai/ha.

### Prohydrojasmon

Compositions and methods of the present disclosure can include prohydrojasmon or an agriculturally acceptable salt or ester thereof. Prohydrojasmon, shown below, is a synthetic plant hormone used for: prevention of rind puffing in Satsuma mandarin oranges and promotion of red color development in apples. Prohydrojasmon, as well as methods of preparing prohydrojasmon, are known in the art. Its plant growth regulation activity is described, for example, in The Pesticide Manual, Eighteenth Edition, 2016.

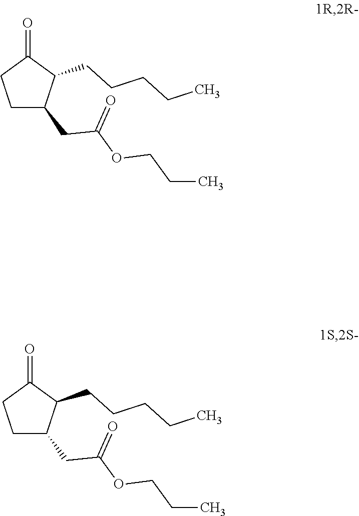 [SMILES: CCCCC[C@H]1C(=O)CC[C@@H]1CC(=O)OCCC ; CCCCC[C@@H]1C(=O)CC[C@H]1CC(=O)OCCC]

### Propham

Compositions and methods of the present disclosure can include propham or an agriculturally acceptable salt or ester thereof. Propham, shown below, is a carbamate used for control of annual grass and some broad-leaved weeds in alfalfa, clover, sugar beets, spinach, lettuce, peas, beans, flax, safflowers, beetroot, fodder beets, lettuce, chard, lentils and perennial grass seed crops, at 2300-5000 g/ha, and as a sprouting inhibitor in potatoes. Propham, as well as methods of preparing propham, are known in the art. Its plant growth regulation activity is described, for example, in The Pesticide Manual, Eighteenth Edition, 2016.

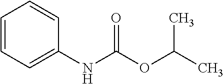

Propham can be applied to vegetation or an area adjacent the vegetation or applied to soil or water to prevent the emergence or growth of vegetation in an amount sufficient to induce a herbicidal effect. In some aspects, propham is applied to vegetation or an area adjacent the vegetation or applied to soil or water to prevent the emergence or growth of vegetation in an amount of 2000 g ai/ha or more, such as 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3750 g ai/ha or more, 4000 g ai/ha or more, 4250 g ai/ha or more, 4500 g ai/ha or more, 4750 g ai/ha or more, 5000 g ai/ha or more, 5250 g ai/ha or more, 5500 g ai/ha or more, 5750 g ai/ha or more, 6000 g ai/ha or more, 6250 g ai/ha or more, 6500 g ai/ha or more, or 6750 g ai/ha or more; in an amount of 7000 g ai/ha or less, such as 6750 g ai/ha or less, 6500 g ai/ha or less, 6250 g ai/ha or less, 6000 g ai/ha or less, 5750 g ai/ha or less, 5500 g ai/ha or less, 5250 g ai/ha or less, 5000 g ai/ha or less, 4750 g ai/ha or less, 4500 g ai/ha or less, 4250 g ai/ha or less, 4000 g ai/ha or less, 3750 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, or 2100 g ai/ha or less; or in an amount ranging from any of the minimum values described above to any of the maximum values described above, such as 2000-7000 g ai/ha, 4750-6500 g ai/ha, 2300-3750 g ai/ha, 4000-6000 g ai/ha, 2700-5000 g ai/ha, 3100-4750 g ai/ha, 2500-4000 g ai/ha, 5750-7000 g ai/ha, 3300-5250 g ai/ha, 2000-4500 g ai/ha, 2900-5500 g ai/ha, or 2100-6750 g ai/ha.

### Sintofen

Compositions and methods of the present disclosure can include sintofen or an agriculturally acceptable salt or ester thereof. Sintofen, shown below, is a carboxylic acid used for sterilizing spring and winter wheat for use in hybridization, at 800 g/ha. Sintofen, as well as methods of preparing sintofen, are known in the art. Its plant growth regulation activity is described, for example, in The Pesticide Manual, Eighteenth Edition, 2016.

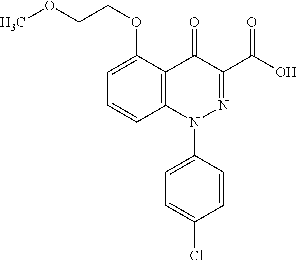 [SMILES: COCCOc1cccc2c1c(=O)c(C(=O)O)nn2-c1ccc(Cl)cc1]

### Thidiazuron

Compositions and methods of the present disclosure can include thidiazuron or an agriculturally acceptable salt or ester thereof. Thidiazuron, shown below, is a carboxylic acid used for defoliation of cotton, at 210 g/ha. Thidiazuron, as well as methods of preparing thidiazuron, are known in the art. Its plant growth regulation activity is described, for example, in The Pesticide Manual, Eighteenth Edition, 2016.

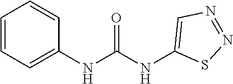 [SMILES: O=C(Nc1ccccc1)Nc1cnns1]

Thidiazuron can be applied to vegetation or an area adjacent the vegetation or applied to soil or water to prevent the emergence or growth of vegetation in an amount sufficient to induce a herbicidal effect. In some aspects, thidiazuron is applied to vegetation or an area adjacent the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, or 340 g ai/ha or more; in an amount of 350 g ai/ha or less, such as 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-350 g ai/ha, 125-330 g ai/ha, 60-300 g ai/ha, 70-250 g ai/ha, 250-340 g ai/ha, 130-190 g ai/ha, 90-240 g ai/ha, 75-220 g ai/ha, 80-325 g ai/ha, 160-200 g ai/ha, 50-240 g ai/ha, or 55-340 g ai/ha.

### Triacontanol

Compositions and methods of the present disclosure can include triacontanol or an agriculturally acceptable salt or ester thereof. Triacontanol, shown below, is a fatty alcohol used to increase the yields in cotton, potato, chilis, tomatoes, rice, grains and peanuts, and to increase the dry matter content, plant height, root spread and uniform, early maturity in crops. Triacontanol, as well as methods of preparing triacontanol, are known in the art. Its plant growth regulation activity is described, for example, in The Pesticide Manual, Eighteenth Edition, 2016.

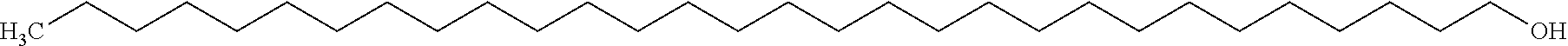 [SMILES: CCCCCCCCCCCCCCCCCCCCCCCCCCCCCCO]

### Trinexapac

Compositions and methods of the present disclosure can include trinexapac or an agriculturally acceptable salt or ester thereof. Trinexapac, shown below, is a cyclohexanedione used to: prevent lodging in cereals and winter oilseed rape, at 100-300 g/ha; retard growth in turf, at 150-500 g/ha; and promote maturation in sugar cane, at 100-250 g/ha. Trinexapac, as well as methods of preparing trinexapac, are known in the art. Its plant growth regulation activity is described, for example, in The Pesticide Manual, Eighteenth Edition, 2016.

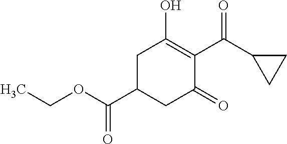 [SMILES: CCOC(=O)C1CC(=O)C(C(=O)C2CC2)=C(O)C1]

Trinexapac can be applied to vegetation or an area adjacent the vegetation or applied to soil or water to prevent the emergence or growth of vegetation in an amount sufficient to induce a herbicidal effect. In some aspects, trinexapac is applied to vegetation or an area adjacent the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, or 575 g ai/ha or more; in an amount of 600 g ai/ha or less, such as 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-600 g ai/ha, 75-500 g ai/ha, 160-375 g ai/ha, 240-500 g ai/ha, 150-370 g ai/ha, 120-450 g ai/ha, 190-400 g ai/ha, 60-220 g ai/ha, 380-525 g ai/ha, 225-420 g ai/ha, 400-550 g ai/ha, or 55-575 g ai/ha.

### Uniconazole

Compositions and methods of the present disclosure can include uniconazole or an agriculturally acceptable salt or ester thereof. Uniconazole, shown below, is a triazole used to: reduce lodging in rice; reduce vegetative growth and increase flowering in ornamentals, at a spray concentration of 0.5-50 ppm; and reduce vegetative growth in trees. Uniconazole-P is an optically active form of uniconazole. Uniconazole, as well as methods of preparing uniconazole, are known in the art. Its plant growth regulation activity is described, for example, in The Pesticide Manual, Eighteenth Edition, 2016.

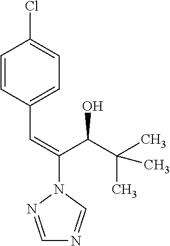 [SMILES: CC(C)(C)[C@H](O)/C(=C\c1ccc(Cl)cc1)n1cncn1]

### Compositions

A composition comprising compounds according to Formula I may be mixed with or applied in combination with (b) a plant growth regulator (PGR), an agriculturally acceptable salt or ester thereof, or mixtures thereof.

In some aspects, (a) and (b) are used in an amount sufficient to induce an unexpectedly enhanced herbicidal effect (e.g., increased damage or injury to undesirable vegetation) while still showing good crop compatibility (e.g., no increased damage to crops or minimal increased damage or injury to crops) when compared to the individual application of the herbicidal compounds (a) or (b). In some aspects, the damage or injury to undesirable vegetation caused by the compositions and methods disclosed herein is evaluated using a scale from 0% to 100%, when compared with the untreated control vegetation, wherein 0% indicates no damage to the undesirable vegetation and 100% indicates complete destruction of the undesirable vegetation.

In some aspects, the joint action of (a) the compounds according to Formula I and (b) the PGR, an agriculturally acceptable salt or ester thereof, or mixtures thereof results in unexpectedly enhanced herbicidal effect against undesirable vegetation, even at application rates below those typically used for the herbicide to have a herbicidal effect on its own. In some aspects, the compositions and methods disclosed herein can, based on the individual components, be used at lower application rates to achieve a herbicidal effect comparable to the effect produced by the individual components at normal application rates.

In some aspects, the weight ratio of (a) the compounds according to Formula I (in g ae/ha) to (b) the PGR, an agriculturally acceptable salt or ester thereof, or mixtures thereof (in g ai/ha) is 1:7500 or more, such as 1:7000 or more, 1:6000 or more, 1:5000 or more, 1:4000 or more, 1:3000 or more, 1:2500 or more, 1:2000 or more, 1:1500 or more, 1:1000 or more, 1:900 or more, 1:800 or more, 1:700 or more, 1:600 or more, 1:500 or more, 1:400 or more, 1:300 or more, 1:250 or more, 1:200 or more, 1:150 or more, 1:100 or more, 1:90 or more, 1:80 or more, 1:75 or more, 1:70 or more, 1:60 or more, 1:50 or more, 1:40 or more, 1:35 or more, 1:30 or more, 1:28 or more, 1:26 or more, 1:25 or more, 1:24 or more, 1:22 or more, 1:21 or more, 1:20 or more, 1:18 or more, 1:16 or more, 1:15 or more, 1:14 or more, 1:12 or more, 1:10 or more, 1:9 or more, 1:8 or more, 1:7 or more, 1:6 or more, 1:5 or more, 1:4 or more, 1:3 or more, 1:2 or more, 1:1.9 or more, 1:1.8 or more, 1:1.7 or more, 1:1.6 or more, 1:1.5 or more, 1:1.4 or more, 1:1.3 or more, 1:1.2 or more, 1:1.1 or more, 1:1 or more, 1.1:1 or more, 1.25:1 or more, 1.5:1 or more, 1.75:1 or more, 2:1 or more, 2.5:1 or more, 3:1 or more, 3.5:1 or more, 4:1 or more, 4.5:1 or more, 5:1 or more, 6:1 or more, 7:1 or more, 8:1 or more, 9:1 or more, 10:1 or more, 11:1 or more, 12:1 or more, 13:1 or more, or 14:1 or more; in some aspects, the weight ratio of (a) to (b) is 15:1 or less, such as 14:1 or less, 13:1 or less, 12:1 or less, 11:1 or less, 10:1 or less, 9:1 or less, 8:1 or less, 7:1 or less, 6:1 or less, 5:1 or less, 4.5:1 or less, 4:1 or less, 3.5:1 or less, 3:1 or less, 2.5:1 or less, 2:1 or less, 1.75:1 or less, 1.5:1 or less, 1.25:1 or less, 1.1:1 or less, 1:1 or less, 1:1.1 or less, 1:1.2 or less, 1:1.3 or less, 1:1.4 or less, 1:1.5 or less, 1:1.6 or less, 1:1.7 or less, 1:1.8 or less, 1:1.9 or less, 1:2 or less, 1:3 or less, 1:4 or less, 1:5 or less, 1:6 or less, 1:7 or less, 1:8 or less, 1:9 or less, 1:10 or less, 1:12 or less, 1:14 or less, 1:15 or less, 1:16 or less, 1:18 or less, 1:20 or less, 1:21 or less, 1:22 or less, 1:24 or less, 1:25 or less, 1:26 or less, 1:28 or less, 1:30 or less, 1:35 or less, 1:40 or less, 1:50 or less, 1:60 or less, 1:70 or less, 1:80 or less, 1:90 or less, 1:100 or less, 1:150 or less, 1:200 or less, 1:250 or less, 1:300 or less, 1:400 or less, 1:500 or less, 1:600 or less, 1:700 or less, 1:800 or less, 1:900 or less, 1:1000 or less, 1:1500 or less, 1:2000 or less, 1:2500 or less, 1:3000 or less, 1:4000 or less, 1:5000 or less, 1:6000 or less, or 1:7000 or less; or the weight ratio of (a) to (b) can range from any of the minimum ratios to any of the maximum ratios provided above, such as from 1:7500 to 15:1, from 1:6000 to 13:1, from 1:4000 to 11:1, from 1:1250 to 9:1, from 1:1500 to 1:50, from 1:20 to 8:1, from 1:8 to 2:1, or from 1:5 to 1:1.

In some aspects, the active ingredients in the compositions disclosed herein consist of compounds according to Formula I and (b) a PGR, an agriculturally acceptable salt or ester thereof, or mixtures thereof. In some aspects, the composition may include other components, such as safeners or adjuvants, but does not include a herbicidal active ingredient in addition to (a) and (b). In some aspects, the compositions may exclude one or more herbicidal active ingredients specified above. In some aspects, the compositions may include one or more herbicidal active ingredients in addition to (a) and (b), but may exclude one or more herbicidal ingredients specified above.

In some aspects, (a) and (b), independently, can be employed in a purity of from 90% to 100% (e.g., from 95% to 100%) according to nuclear magnetic resonance (NMR) spectroscopy.

### ACCase Inhibitor Herbicides

In addition to the compounds according to Formula I or agriculturally acceptable N-oxide, salt or ester thereof, the compositions include an acetyl CoA carboxylase (ACCase) inhibitor herbicide or an agriculturally acceptable salt or ester thereof. ACCase inhibitor herbicides inhibit lipid biosynthesis in the plant. Examples of ACCase inhibitor herbicides include aryloxyphenoxypropionates, cyclohexanediones, and phenylpyrazolines. In some aspects, the ACCase inhibitor herbicide can include an aryloxyphenoxypropionate herbicide. In some aspects, the ACCase inhibitor herbicide can include a cyclohexanedione herbicide. In some aspects, the ACCase inhibitor herbicide can include a phenylpyrazoline herbicide.

In some aspects, the composition can include an ACCase inhibitor selected from the group of cyclohexanediones, aryloxyphenoxypropionates, phenylpyrazolines, or combinations thereof. In some cases, the composition can include clodinafop, cyhalofop, diclofop, fenoxaprop, fenthiaprop, fluazifop, haloxyfop, metamifop, propaquizafop, quizalofop, agriculturally acceptable salts or esters thereof, or combinations thereof. In some cases, the composition can include, alloxydim, butroxydim, clethodim, cloproxydim, cycloxydim, profoxydim, sethoxydim, tepraloxydim, tralkoxydim, agriculturally acceptable salts or esters thereof, or combinations thereof. In some cases, the composition can include pinoxaden

In some aspects, the composition can include an ACCase inhibitor herbicide selected from the group consisting of alloxydim, butroxydim, clethodim, clodinafop, cloproxydim, cycloxydim, cyhalofop, diclofop, fenoxaprop, fenthiaprop, fluazifop, haloxyfop, metamifop, pinoxaden, profoxydim, propaquizafop, quizalofop, sethoxydim, tepraloxydim, tralkoxydim, agriculturally acceptable salts and esters thereof, and combinations thereof.

The ACCase inhibitor herbicide or agriculturally acceptable salt or ester thereof can be applied to vegetation or an area adjacent the vegetation or applied to soil or water to prevent the emergence or growth of vegetation in an amount sufficient to induce a herbicidal effect. In some aspects, the ACCase inhibitor herbicide or agriculturally acceptable salt or ester thereof is applied to vegetation or an area adjacent the vegetation or applied to soil or water to prevent the emergence or growth of vegetation in an amount of 2 g ai/ha or more, such as 2.5 g ai/ha or more, 3 g ai/ha or more, 3.5 g ai/ha or more, 4 g ai/ha or more, 4.5 g ai/ha or more, 5 g ai/ha or more, 6 g ai/ha or more, 7 g ai/ha or more, 8 g ai/ha or more, 9 g ai/ha or more, 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 250 g ai/ha or more, 260 g ai/ha or more, 280 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, or 1450 g ai/ha or more; in an amount of 1500 g ai/ha or less, such as 1450 g ai/ha or less, 1400 g ai/ha or less, 1350 g ai/ha or less, 1300 g ai/ha or less, 1250 g ai/ha or less, 1240 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 380 g ai/ha or less, 360 g ai/ha or less, 350 g ai/ha or less, 340 g ai/ha or less, 320 g ai/ha or less, 300 g ai/ha or less, 280 g ai/ha or less, 260 g ai/ha or less, 250 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 10 g ai/ha or less, 9 g ai/ha or less, 8 g ai/ha or less, 7 g ai/ha or less, 6 g ai/ha or less, 5 g ai/ha or less, 4.5 g ai/ha or less, 4 g ai/ha or less, 3.5 g ai/ha or less, 3 g ai/ha or less, or 2.5 g ai/ha or less; or in an amount ranging from any of the minimum values described above to any of the maximum values described above, such as 2-1500 g ai/ha, 5-1300 g ai/ha, 6-250 g ai/ha, 75-1240 g ai/ha, 90-900 g ai/ha, 55-290 g ai/ha, 7-600 g ai/ha, 20-700 g ai/ha, 190-1450 g ai/ha, 65-1100 g ai/ha, 320-1000 g ai/ha, 250-750 g ai/ha, 700-1200 g ai/ha, 850-1400 g ai/ha, or 2.5-1450 g ai/ha.

In some aspects, the composition contains: compounds according to Formula I; and (b) alloxydim, butroxydim, clethodim, clodinafop, cloproxydim, cycloxydim, cyhalofop, diclofop, fenoxaprop, fenthiaprop, fluazifop, haloxyfop, metamifop, pinoxaden, profoxydim, propaquizafop, quizalofop, sethoxydim, tepraloxydim, tralkoxydim, agriculturally acceptable salts and esters thereof, and combinations thereof.

### Alloxydim

Compositions and methods of the present disclosure can include alloxydim or an agriculturally acceptable salt or ester thereof. Alloxydim, shown below, is a cyclohexanedione herbicide that provides post-emergence control of grass weeds and volunteer cereals in sugar beet, vegetables and broad-leaved crops. Alloxydim, as well as methods of preparing alloxydim, are known in the art. Its herbicidal activity is described in The Pesticide Manual, Seventeenth Edition, 2016.

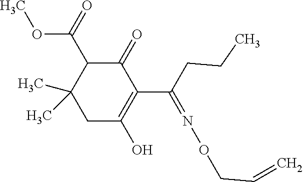 [SMILES: C=CCON=C(CCC)C1=C(O)CC(C)(C)C(C(=O)OC)C1=O]

Alloxydim can be applied to vegetation or an area adjacent the vegetation or applied to soil or water to prevent the emergence or growth of vegetation in an amount sufficient to induce a herbicidal effect. In some aspects, alloxydim is applied to vegetation or an area adjacent the vegetation or applied to soil or water to prevent the emergence or growth of vegetation in an amount of 299 g ai/ha or more, such as 300 g ai/ha or more, 320 g ai/ha or more, 340 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 620 g ai/ha or more, 640 g ai/ha or more, 660 g ai/ha or more, 680 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1200 g ai/ha or more, 1300 g ai/ha or more, 1400 g ai/ha or more, or 1500 g ai/ha or more; in an amount of 1501 g ai/ha or less, such as 1500 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 680 g ai/ha or less, 660 g ai/ha or less, 640 g ai/ha or less, 620 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, or 300 g ai/ha or less; or from any of the minimum values described above to any of the maximum values described above, such as 299-1501 g ai/ha, 380-1250 g ai/ha, 440-850 g ai/ha, 700-1000 g ai/ha, 320-950 g ai/ha, 680-1350 g ai/ha, 400-1050 g ai/ha, 500-1000 g ai/ha, 600-900 g ai/ha, 700-1300 g ai/ha, 500-700 g ai/ha, or 320-1400 g ai/ha.

### Butroxydim

Compositions and methods of the present disclosure can include butroxydim or an agriculturally acceptable salt or ester thereof. Butroxydim, shown below, is a cyclohexanedione herbicide that provides post-emergence control of annual grasses in many broad-leaved crops. Butroxydim, as well as methods of preparing butroxydim, are known in the art. Its herbicidal activity is described in The Pesticide Manual, Seventeenth Edition, 2016.

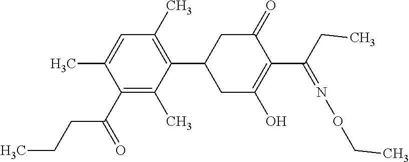 [SMILES: CCCC(=O)c1c(C)cc(C)c(C2CC(=O)C(C(CC)=NOCC)=C(O)C2)c1C]

Butroxydim can be applied to vegetation or an area adjacent the vegetation or applied to soil or water to prevent the emergence or growth of vegetation in an amount sufficient to induce a herbicidal effect. In some aspects, butroxydim is applied to vegetation or an area adjacent the vegetation or applied to soil or water to prevent the emergence or growth of vegetation in an amount of 9 g ai/ha or more, such as 10 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 105 g ai/ha or more, 110 g ai/ha or more, 115 g ai/ha or more, or 120 g ai/ha or more; in an amount of 121 g ai/ha or less, such as 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, or 10 g ai/ha or less; or in an amount ranging from any of the minimum values described above to any of the maximum values described above, such as 9-121 g ai/ha, 10-115 g ai/ha, 20-110 g ai/ha, 15-85 g ai/ha, 30-100 g ai/ha, 50-95 g ai/ha, 15-60 g ai/ha, 25-85 g ai/ha, 40-80 g ai/ha, 20-75 g ai/ha, 35-110 g ai/ha, 25-105 g ai/ha, 15-55 g ai/ha, or 10-120 g ai/ha.

### Clethodim

Compositions and methods of the present disclosure can include clethodim or an agriculturally acceptable salt or ester thereof. Clethodim, shown below, is a cyclohexanedione herbicide that provides post-emergence control of annual and perennial grasses in a wide range of broad-leaved crops, including such field crops as canola/oilseed rape, soybean, cotton, flax, sunflowers, alfalfa, peanuts, sugar beet, tobacco, and potatoes, as well as vegetable crops, trees, and vines. Clethodim, as well as methods of preparing clethodim, are known in the art. Its herbicidal activity is described in The Pesticide Manual, Seventeenth Edition, 2016.

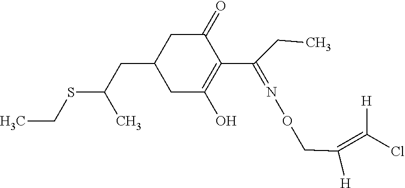 [SMILES: CCSC(C)CC1CC(=O)C(C(CC)=NOC/C=C/Cl)=C(O)C1]

Clethodim can be applied to vegetation or an area adjacent the vegetation or applied to soil or water to prevent the emergence or growth of vegetation in an amount sufficient to induce a herbicidal effect. In some aspects, clethodim is applied to vegetation or an area adjacent the vegetation or applied to soil or water to prevent the emergence or growth of vegetation in an amount of 2 g ai/ha or more, such as 3 g ai/ha or more, 4 g ai/ha or more, 6 g ai/ha or more, 8 g ai/ha or more, 10 g ai/ha or more, 12 g ai/ha or more, 14 g ai/ha or more, 16 g ai/ha or more, 18 g ai/ha or more, 20 g ai/ha or more, 22 g ai/ha or more, 24 g ai/ha or more, 26 g ai/ha or more, 28 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 80 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 620 g ai/ha or more, 640 g ai/ha or more, 660 g ai/ha or more, 680 g ai/ha or more, 700 g ai/ha or more, 750 g ai/ha or more, 800 g ai/ha or more, 850 g ai/ha or more, or 875 g ai/ha or more; in an amount of 900 g ai/ha or less, such as 875 g ai/ha or less, 850 g ai/ha or less, 800 g ai/ha or less, 750 g ai/ha or less, 700 g ai/ha or less, 680 g ai/ha or less, 660 g ai/ha or less, 640 g ai/ha or less, 620 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 235 g ai/ha or less, 230 g ai/ha or less, 225 g ai/ha or less, 220 g ai/ha or less, 215 g ai/ha or less, 210 g ai/ha or less, 205 g ai/ha or less, 200 g ai/ha or less, 195 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 80 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 28 g ai/ha or less, 26 g ai/ha or less, 24 g ai/ha or less, 22 g ai/ha or less, 20 g ai/ha or less, 18 g ai/ha or less, 16 g ai/ha or less, 14 g ai/ha or less, 12 g ai/ha or less, 10 g ai/ha or less, 8 g ai/ha or less, 6 g ai/ha or less, 4 g ai/ha or less, or 3 g ai/ha or less, or in an amount ranging from any of the minimum values described above to any of the maximum values described above, such as 2-900 g ai/ha, 3-800 g ai/ha, 6-700 g ai/ha, 22-500 g ai/ha, 12-600 g ai/ha, 5-500 g ai/ha, 40-440 g ai/ha, 20-700 g ai/ha, 80-400 g ai/ha, 60-270 g ai/ha, 80-750 g ai/ha, 100-290 g ai/ha, 175-750 g ai/ha, 300-900 g ai/ha, 3-100 g ai/ha, 35-320 g ai/ha, 3-205 g ai/ha, 4-50 g ai/ha, or 3-875 g ai/ha.

### Clodinafop

Compositions and methods of the present disclosure can include clodinafop or an agriculturally acceptable salt or ester thereof. Clodinafop-propargyl, shown below, is prop-2-ynyl (R)-2-[4-(5-chloro-3-fluoropyridin-2-yloxy) phenoxy]propionate and is used for post-emergence control of annual grasses, including Avena, Lolium, Setaria, Phalaris, and Alopecurus spp., in cereals.

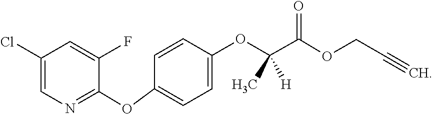 [SMILES: C#CCOC(=O)[C@@H](C)Oc1ccc(Oc2ncc(Cl)cc2F)cc1]

Clodinafop can be applied to vegetation or an area adjacent the vegetation or applied to soil or water to prevent the emergence or growth of vegetation in an amount sufficient to induce a herbicidal effect. In some aspects, clodinafop is applied to vegetation or an area adjacent the vegetation or applied to soil or water to prevent the emergence or growth of vegetation in an amount of 10 g ai/ha or more, such as 11 g ai/ha or more, 12 g ai/ha or more, 13 g ai/ha or more, 14 g ai/ha or more, 15 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, or 95 g ai/ha or more; in an amount of 100 g ai/ha or less, such as 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 15 g ai/ha or less, 14 g ai/ha or less, 13 g ai/ha or less, 12 g ai/ha or less, or 11 g ai/ha or less; or in an amount ranging from any of the minimum values described above to any of the maximum values described above, such as 10-100 g ai/ha, 15-90 g ai/ha, 12-80 g ai/ha, 25-70 g ai/ha, 14-40 g ai/ha, 20-85 g ai/ha, 30-60 g ai/ha, 40-90 g ai/ha, 50-100 g ai/ha, 20-90 g ai/ha, 50-95 g ai/ha, 30-90 g ai/ha, 20-60 g ai/ha, 30-80 g ai/ha, 13-75 g ai/ha, or 11-95 g ai/ha.

### Cloproxydim

Compositions and methods of the present disclosure can include cloproxydim or an agriculturally acceptable salt or ester thereof. Cloproxydim, shown below, is a cyclohexanedione herbicide that has been evaluated by the Chevron Chemical Company. Cloproxydim provides post-emergence control of a wide range of annual and perennial grasses in

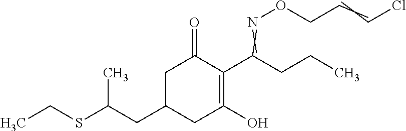 [SMILES: CCCC(=NOCC=CCl)C1=C(O)CC(CC(C)SCC)CC1=O]

beans, cotton, potatoes, sugar beets, and most vegetable crops. Its herbicidal activity is described in The Pesticide Manual, Seventeenth Edition, 2016.

### Cycloxydim

Compositions and methods of the present disclosure can include cycloxydim or an agriculturally acceptable salt or ester thereof. Cycloxydim, shown below, is a cyclohexanedione herbicide that provides post-emergence control of annual and perennial grasses (except red fescue and Poa spp.) in canola/oilseed rape, beans, potatoes, brassicas, cotton, celery, fennel, beetroot, sugar beets, fodder beets, sunflowers, soybeans, flax, alfalfa, crucifers, and alliums. Cycloxydim, as well as methods of preparing cycloxydim, are known in the art. Its herbicidal activity is described in The Pesticide Manual, Seventeenth Edition, 2016.

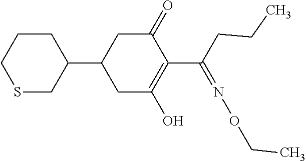 [SMILES: CCCC(=NOCC)C1=C(O)CC(C2CCCSC2)CC1=O]

Cycloxydim can be applied to vegetation or an area adjacent the vegetation or applied to soil or water to prevent the emergence or growth of vegetation in an amount sufficient to induce a herbicidal effect. In some aspects, cycloxydim is applied to vegetation or an area adjacent the vegetation or applied to soil or water to prevent the emergence or growth of vegetation in an amount of 50 g ai/ha or more, such as 55 g ai/ha or more, 60 g ai/ha or more, 70 g ai/ha or more, 80 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, or 675 g ai/ha or more; in an amount of 700 g ai/ha or less, such as 675 g ai/ha or less, 650 g ai/ha or less, 640 g ai/ha or less, 620 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 195 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 145 g ai/ha or less, 140 g ai/ha or less, 135 g ai/ha or less, 130 g ai/ha or less, 125 g ai/ha or less, 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-700 g ai/ha, 60-600 g ai/ha, 90-400 g ai/ha, 100-580 g ai/ha, 50-300 g ai/ha, 75-460 g ai/ha, 110-620 g ai/ha, 190-280 g ai/ha, 140-400 g ai/ha, 95-660 g ai/ha, 80-580 g ai/ha, 200-700 g ai/ha, 250-300 g ai/ha, 65-400 g ai/ha, 90-320 g ai/ha, or 55-675 g ai/ha.

### Cyhalofop

Compositions and methods of the present disclosure can include cyhalofop or an agriculturally acceptable salt or ester thereof. Cyhalofop, as well as methods of preparing cyhalofop, are known in the art. Cyhalofop is an aryloxyphenoxypropionate ester that is used for post-emergence control of grass weeds in rice. Cyhalofop-butyl, shown below, is an ester of cyhalofop.

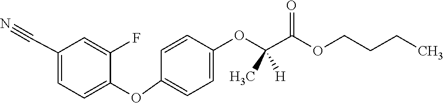 [SMILES: CCCCOC(=O)[C@@H](C)Oc1ccc(Oc2ccc(C#N)cc2F)cc1]

Cyhalofop can be applied to vegetation or an area adjacent the vegetation or applied to soil or water to prevent the emergence or growth of vegetation in an amount sufficient to induce a herbicidal effect. In some aspects, cyhalofop is applied to vegetation or an area adjacent the vegetation or applied to soil or water to prevent the emergence or growth of vegetation in an amount of 20 g ai/ha or more, such as 25 g ai/ha or more, 30 g ai/ha or more, 35 g ai/ha or more, 40 g ai/ha or more, 50 g ai/ha or more, 60 g ai/ha or more, 70 g ai/ha or more, 80 g ai/ha or more, 90 g ai/ha or more, 100 g ai/ha or more, 120 g ai/ha or more, 140 g ai/ha or more, 150 g ai/ha or more, 160 g ai/ha or more, 180 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, or 500 g ai/ha or more; in an amount of 500 g ai/ha or less, such as 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 180 g ai/ha or less, 160 g ai/ha or less, 150 g ai/ha or less, 140 g ai/ha or less, 120 g ai/ha or less, 100 g ai/ha or less, 90 g ai/ha or less, 80 g ai/ha or less, 70 g ai/ha or less, 60 g ai/ha or less, 50 g ai/ha or less, 40 g ai/ha or less, 35 g ai/ha or less, 30 g ai/ha or less, or 25 g ai/ha or less; or in an amount ranging from any of the minimum values described above to any of the maximum values described above, such as 20-500 g ai/ha, 20-450 g ai/ha, 25-200 g ai/ha, 40-400 g ai/ha, 60-375 g ai/ha, 40-350 g ai/ha, 50-250 g ai/ha, 60-325 g ai/ha, 90-275 g ai/ha, 70-325 g ai/ha, 40-225 g ai/ha, 140-300 g ai/ha, 35-160 g ai/ha, 80-350 g ai/ha, 150-450 g ai/ha, 200-425 g ai/ha, or 25-475 g ai/ha.

### Diclofop

Compositions and methods of the present disclosure can include diclofop or an agriculturally acceptable salt or ester thereof. Diclofop-methyl, shown below, is an aryloxyphenoxypropionate that is used for post-emergence control of wild oats, wild millets, and other annual grass weeds in wheat, barley, rye, red fescue, and broad-leaved crops such as soybeans, sugar beets, fodder beets, flax, legumes, canola/oilseed rape, sunflowers, clover, alfalfa, peanuts, brassicas, carrots, celery, parsnips, lettuce, spinach, potatoes, cucumbers, peas, beans, tomatoes, fennel, alliums, and herbs. Diclofop, as well as methods of preparing diclofop, are known in the art.

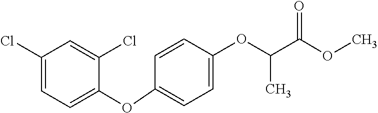 [SMILES: COC(=O)C(C)Oc1ccc(Oc2ccc(Cl)cc2Cl)cc1]

Diclofop can be applied to vegetation or an area adjacent the vegetation or applied to soil or water to prevent the emergence or growth of vegetation in an amount sufficient to induce a herbicidal effect. In some aspects, diclofop is applied to vegetation or an area adjacent the vegetation or applied to soil or water to prevent the emergence or growth of vegetation in an amount 100 g ai/ha or more, such as 150 g ai/ha or more, 175 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 620 g ai/ha or more, 640 g ai/ha or more, 660 g ai/ha or more, 680 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, or 1200 g ai/ha or more; in an amount of 1200 g ai/ha or less, such as 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 680 g ai/ha or less, 660 g ai/ha or less, 640 g ai/ha or less, 620 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 280 g ai/ha or less, 260 g ai/ha or less, 240 g ai/ha or less, 220 g ai/ha or less, 200 g ai/ha or less, 175 g ai/ha or less, 150 g ai/ha or less, 140 g ai/ha or less, 130 g ai/ha or less, 120 g ai/ha or less, 110 g ai/ha or less, or 101 g ai/ha or less; or in an amount ranging from any of the minimum values described above to any of the maximum values described above, such as 100-1200 g ai/ha, 120-1200 g ai/ha, 140-1100 g ai/ha, 150-900 g ai/ha, 175-1000 g ai/ha, 200-1150 g ai/ha, 360-1050 g ai/ha, 220-640 g ai/ha, 240-800 g ai/ha, 110-700 g ai/ha, 300-440 g ai/ha, 750-1000 g ai/ha, 360-950 g ai/ha, 175-850 g ai/ha, 400-650 g ai/ha, 150-280 g ai/ha, 500-600 g ai/ha, or 110-1150 g ai/ha).

### Fenoxaprop

Compositions and methods of the present disclosure can include fenoxaprop or an agriculturally acceptable salt or ester thereof. Fenoxaprop is an aryloxyphenoxypropionate that is used for post-emergence control of annual and perennial grass weeds in potatoes, beans, soybeans, beets, vegetables, peanuts, and cotton; and (when applied with the herbicide safener fenchlorazole-ethyl) annual and perennial grass weeds and wild oats in wheat, rye, and triticale. Fenoxaprop-P-ethyl, shown below, is an ester of fenoxaprop. Fenoxaprop, as well as methods of preparing fenoxaprop, are known in the art.

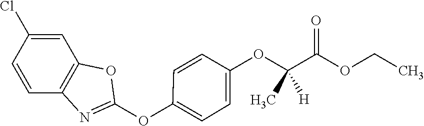 [SMILES: CCOC(=O)[C@@H](C)Oc1ccc(Oc2nc3ccc(Cl)cc3o2)cc1]

Fenoxaprop can be applied to vegetation or an area adjacent the vegetation or applied to soil or water to prevent the emergence or growth of vegetation in an amount sufficient to induce a herbicidal effect. In some aspects, fenoxaprop is applied to vegetation or an area adjacent the vegetation or applied to soil or water to prevent the emergence or growth of vegetation in an amount of 25 g ai/ha or more, such as 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 105 g ai/ha or more, 110 g ai/ha or more, 115 g ai/ha or more, 120 g ai/ha or more, 125 g ai/ha or more, 130 g ai/ha or more, 135 g ai/ha or more, 140 g ai/ha or more, 145 g ai/ha or more, or 150 g ai/ha or more; in an amount of 151 g ai/ha or less, such as 150 g ai/ha or less, 145 g ai/ha or less, 140 g ai/ha or less, 135 g ai/ha or less, 130 g ai/ha or less, 125 g ai/ha or less, 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 25 g ai/ha or less; or in an amount ranging from any of the minimum values described above to any of the maximum values described above, such as 25-151 g ai/ha, 30-150 g ai/ha, 35-140 g ai/ha, 50-130 g ai/ha, 30-125 g ai/ha, 85-150 g ai/ha, 45-145 g ai/ha, 25-110 g ai/ha, 40-90 g ai/ha, 55-120 g ai/ha, 40-115 g ai/ha, 60-110 g ai/ha, 30-105 g ai/ha, 90-151 g ai/ha, 25-95 g ai/ha, 50-80 g ai/ha, 60-125 g ai/ha, or 25-145 g ai/ha.

### Fenthiaprop

Compositions and methods of the present disclosure can include fenthiaprop or an agriculturally acceptable salt or ester thereof. Fenthiaprop, shown below, is an aryloxyphenoxypropionate herbicide that is used for post-emergence control of many annual and perennial grasses and volunteer cereals in canola/oilseed rape, potatoes, and sugar beets.

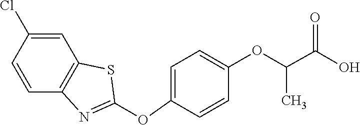 [SMILES: CC(Oc1ccc(Oc2nc3ccc(Cl)cc3s2)cc1)C(=O)O]

Fenthiaprop can be applied to vegetation or an area adjacent the vegetation or applied to soil or water to prevent the emergence or growth of vegetation in an amount sufficient to induce a herbicidal effect. In some aspects, fenthiaprop is applied to vegetation or an area adjacent the vegetation or applied to soil or water to prevent the emergence or growth of vegetation in an amount 150 g ai/ha or more, such as 160 g ai/ha or more, 170 g ai/ha or more, 180 g ai/ha or more, 190 g ai/ha or more, 200 g ai/ha or more, 220 g ai/ha or more, 240 g ai/ha or more, 250 g ai/ha or more, 260 g ai/ha or more, 280 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 420 g ai/ha or more, 440 g ai/ha or more, 450 g ai/ha or more, 460 g ai/ha or more, 480 g ai/ha or more, 500 g ai/ha or more, 520 g ai/ha or more, 540 g ai/ha or more, 550 g ai/ha or more, 560 g ai/ha or more, 580 g ai/ha or more, 600 g ai/ha or more, 620 g ai/ha or more, 640 g ai/ha or more, 650 g ai/ha or more, 660 g ai/ha or more, 680 g ai/ha or more, 700 g ai/ha or more, or 725 g ai/ha or more; in an amount of 750 g ai/ha or less, such as 725 g ai/ha or less, 700 g ai/ha or less, 680 g ai/ha or less, 660 g ai/ha or less, 650 g ai/ha or less, 640 g ai/ha or less, 620 g ai/ha or less, 600 g ai/ha or less, 580 g ai/ha or less, 560 g ai/ha or less, 550 g ai/ha or less, 540 g ai/ha or less, 520 g ai/ha or less, 500 g ai/ha or less, 480 g ai/ha or less, 460 g ai/ha or less, 450 g ai/ha or less, 440 g ai/ha or less, 420 g ai/ha or less, 400 g ai/ha or less, 380 g ai/ha or less, 360 g ai/ha or less, 350 g ai/ha or less, 340 g ai/ha or less, 320 g ai/ha or less, 300 g ai/ha or less, 280 g ai/ha or less, 260 g ai/ha or less, 250 g ai/ha or less, 240 g ai/ha or less, 220 g ai/ha or less, 200 g ai/ha or less, 190 g ai/ha or less, 180 g ai/ha or less, 170 g ai/ha or less, or 160 g ai/ha or less; or in an amount ranging from any of the minimum values described above to any of the maximum values described above, such as 150-750 g ai/ha, 180-600 g ai/ha, 170-650 g ai/ha, 250-700 g ai/ha, 280-450 g ai/ha, 360-580 g ai/ha, 220-640 g ai/ha, 240-725 g ai/ha, 340-440 g ai/ha, 260-550 g ai/ha, 170-250 g ai/ha, 400-650 g ai/ha, 190-580 g ai/ha, 500-600 g ai/ha, or 160-725 g ai/ha.

### Fluazifop

Compositions and methods of the present disclosure can include fluazifop or an agriculturally acceptable salt or ester thereof. Fluazifop is an aryloxyphenoxypropionate that is used for post-emergence control of annual and perennial grass weeds in broad-leaved crops, including control of volunteer cereals and other grass weeds in canola/oilseed rape, sugar beet, fodder beet, potatoes, cotton, soybeans, peanuts, pome fruit, bush fruit, vines, citrus fruit, pineapples, bananas, strawberries, sunflowers, alfalfa, coffee, ornamentals and many vegetables. Fluazifop-P-butyl, shown below, is an ester of fluazifop. Fluazifop, as well as methods of preparing fluazifop, are known in the art.

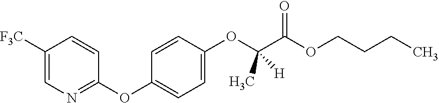 [SMILES: CCCCOC(=O)[C@@H](C)Oc1ccc(Oc2ccc(C(F)(F)F)cn2)cc1]

Fluazifop can be applied to vegetation or an area adjacent the vegetation or applied to soil or water to prevent the emergence or growth of vegetation in an amount sufficient to induce a herbicidal effect. In some aspects, fluazifop is applied to vegetation or an area adjacent the vegetation or applied to soil or water to prevent the emergence or growth of vegetation in an amount of 100 grams active ingredient per hectare (g ai/ha) or more, such as 105 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, or 1350 g ai/ha or more; in an amount of 1400 g ai/ha or less, such as 1350 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less or 105 g ai/ha or less; or in an amount ranging from any of the minimum values described above to any of the maximum values described above, e.g., 100-1400 g ai/ha, 140-1300 g ai/ha, 180-1200 g ai/ha, 200-950 g ai/ha, 300-800 g ai/ha, 250-700 g ai/ha, 130-450 g ai/ha, 100-500 g ai/ha, 190-850 g ai/ha, 110-375 g ai/ha, 225-475 g ai/ha, 130-600 g ai/ha, 150-1200 g ai/ha, 190-700 g ai/ha, 175-350 g ai/ha, 500-1100 g ai/ha, or 105-1350 g ai/ha.

### Haloxyfop

Compositions and methods of the present disclosure can include haloxyfop or an agriculturally acceptable salt or ester thereof. Haloxyfop is an aryloxyphenoxypropionate that is used post-emergence for control of annual and perennial grasses in sugar beets, fodder beets, canola/oilseed rape, potatoes, leafy vegetables, onions, flax, sunflowers, soybeans, vines, strawberries and other crops. Haloxyfop-P-methyl, shown below, is an ester of haloxyfop; haloxyfop-R-methyl is the racemic form of this herbicide. Haloxyfop, as well as methods of preparing haloxyfop, are known in the art.

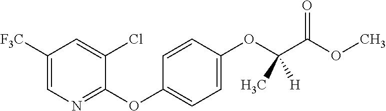 [SMILES: COC(=O)[C@@H](C)Oc1ccc(Oc2ncc(C(F)(F)F)cc2Cl)cc1]

Haloxyfop can be applied to vegetation or an area adjacent the vegetation or applied to soil or water to prevent the emergence or growth of vegetation in an amount sufficient to induce a herbicidal effect. In some aspects, haloxyfop is applied to vegetation or an area adjacent the vegetation or applied to soil or water to prevent the emergence or growth of vegetation in an amount of 24 g ai/ha or more, such as 25 g ai/ha or more, 30 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 210 g ai/ha or more, 220 g ai/ha or more, 230 g ai/ha or more, 240 g ai/ha or more, or 245 g ai/ha or more;

in an amount of 250 g ai/ha or less, such as 245 g ai/ha or less, 240 g ai/ha or less, 230 g ai/ha or less, 220 g ai/ha or less, 210 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 25 g ai/ha or less; in an amount ranging from any of the minimum values described above to any of the maximum values described above, such as 24-250 g ai/ha, 25-225 g ai/ha, 35-200 g ai/ha, 55-190 g ai/ha, 25-180 g ai/ha, 30-175 g ai/ha, 30-230 g ai/ha, 130-220 g ai/ha, 60-160 g ai/ha, 75-150 g ai/ha, 35-150 g ai/ha, 60-140 g ai/ha, 80-230 g ai/ha, 90-210 g ai/ha, 55-110 g ai/ha, 70-240 g ai/ha, or 25-245 g ai/ha.

### Metamifop

Compositions and methods of the present disclosure can include metamifop. Metamifop, shown below, is an aryloxyphenoxypropionate that is used for post-emergence control of annual and perennial grass weeds (including Echinochloa spp., Leptochloa chinensis, Digitaria spp. and Eleusine indica) in rice, wheat, soybeans, cotton, and turf grass. Metamifop, as well as methods of preparing metamifop, are known in the art.

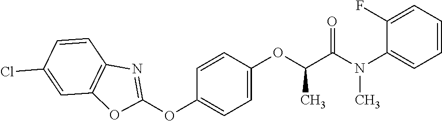 [SMILES: C[C@@H](Oc1ccc(Oc2nc3ccc(Cl)cc3o2)cc1)C(=O)N(C)c1ccccc1F]

Metamifop can be applied to vegetation or an area adjacent the vegetation or applied to soil or water to prevent the emergence or growth of vegetation in an amount sufficient to induce a herbicidal effect. In some aspects, metamifop is applied to vegetation or an area adjacent the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 105 g ai/ha or more, 110 g ai/ha or more, 115 g ai/ha or more, 120 g ai/ha or more, 125 g ai/ha or more, 130 g ai/ha or more, 135 g ai/ha or more, 140 g ai/ha or more, 145 g ai/ha or more, 150 g ai/ha or more, 155 g ai/ha or more, 160 g ai/ha or more, 165 g ai/ha or more, 170 g ai/ha or more, 175 g ai/ha or more, 180 g ai/ha or more, 185 g ai/ha or more, 190 g ai/ha or more, 195 g ai/ha or more, 200 g ai/ha or more, 210 g ai/ha or more, 220 g ai/ha or more, 230 g ai/ha or more, 240 g ai/ha or more, 250 g ai/ha or more, 260 g ai/ha or more, 270 g ai/ha or more, 280 g ai/ha or more, 290 g ai/ha or more, or 295 g ai/ha or more; in an amount of 300 g ai/ha or less, such as 295 g ai/ha or less, 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 195 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 145 g ai/ha or less, 140 g ai/ha or less, 135 g ai/ha or less, 130 g ai/ha or less, 125 g ai/ha or less, 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-300 g ai/ha, 60-275 g ai/ha, 80-250 g ai/ha, 55-125 g ai/ha, 60-200 g ai/ha, 70-160 g ai/ha, 80-210 g ai/ha, 90-270 g ai/ha, 100-175 g ai/ha, 120-230 g ai/ha, 145-250 g ai/ha, or 55-295 g ai/ha.

### Pinoxaden

Compositions and methods of the present disclosure can include pinoxaden or an agriculturally acceptable salt or ester thereof. Pinoxaden, shown below, is a phenylpyrazoline herbicide that provides post-emergence control of grass weeds in wheat, barley, lawns, and turf. Pinoxaden, as well as methods of preparing pinoxaden, are known in the art. Its herbicidal activity is described in The Pesticide Manual, Seventeenth Edition, 2016.

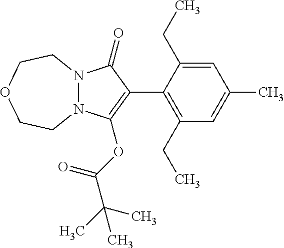 [SMILES: CCc1cc(C)cc(CC)c1-c1c(OC(=O)C(C)(C)C)n2n(c1=O)CCOCC2]

Pinoxaden can be applied to vegetation or an area adjacent the vegetation or applied to soil or water to prevent the emergence or growth of vegetation in an amount sufficient to induce a herbicidal effect. In some aspects, pinoxaden is applied to vegetation or an area adjacent the vegetation or applied to soil or water to prevent the emergence or growth of vegetation in an amount of 2 g ai/ha or more, such as 2.5 g ai/ha or more, 3 g ai/ha or more, 4 g ai/ha or more, 5 g ai/ha or more, 6 g ai/ha or more, 8 g ai/ha or more, 10 g ai/ha or more, 12 g ai/ha or more, 14 g ai/ha or more, 16 g ai/ha or more, 18 g ai/ha or more, 20 g ai/ha or more, 22 g ai/ha or more, 24 g ai/ha or more, 26 g ai/ha or more, 28 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 80 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, or 490 g ai/ha or more; in an amount of 500 g ai/ha or less, such as 490 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 235 g ai/ha or less, 230 g ai/ha or less, 225 g ai/ha or less, 220 g ai/ha or less, 215 g ai/ha or less, 210 g ai/ha or less, 205 g ai/ha or less, 200 g ai/ha or less, 195 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 80 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 28 g ai/ha or less, 26 g ai/ha or less, 24 g ai/ha or less, 22 g ai/ha or less, 20 g ai/ha or less, 18 g ai/ha or less, 16 g ai/ha or less, 14 g ai/ha or less, 12 g ai/ha or less, 10 g ai/ha or less, 8 g ai/ha or less, 6 g ai/ha or less, 5 g ai/ha or less, 4 g ai/ha or less, 3 g ai/ha or less, or 2.5 g ai/ha or less; or in an amount ranging from any of the minimum values described above to any of the maximum values described above, such as 2-500 g ai/ha, 3-400 g ai/ha, 5-300 g ai/ha, 8-100 g ai/ha, 5-500 g ai/ha, 10-120 g ai/ha, 22-190 g ai/ha, 40-300 g ai/ha, 30-400 g ai/ha, 80-270 g ai/ha, 100-480 g ai/ha, 150-250 g ai/ha, 110-380 g ai/ha, 8-235 g ai/ha, 30-260 g ai/ha, 24-50 g ai/ha, 10-300 g ai/ha, 16-180 g ai/ha, or 2.5-490 g ai/ha.

### Profoxydim

Compositions and methods of the present disclosure can include profoxydim or an agriculturally acceptable salt or ester thereof. Profoxydim, shown below, is a cyclohexanedione herbicide. Profoxydim is used for control of grass weeds, including Echinochloa spp., Brachiaria spp., Digitaria spp., Eleusine spp., Leptochloa spp., Setaria spp., Ischaemum rugosum and Rottboellia exaltata, in rice. Profoxydim, as well as methods of preparing profoxydim, are known in the art. Its herbicidal activity is described in The Pesticide Manual, Seventeenth Edition, 2016.

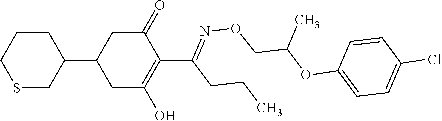 [SMILES: CCC/C(=N\OCC(C)Oc1ccc(Cl)cc1)C1=C(O)CC(C2CCCSC2)CC1=O]

Profoxydim can be applied to vegetation or an area adjacent the vegetation or applied to soil or water to prevent the emergence or growth of vegetation in an amount sufficient to induce a herbicidal effect. In some aspects, profoxydim is applied to vegetation or an area adjacent the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 105 g ai/ha or more, 110 g ai/ha or more, 115 g ai/ha or more, 120 g ai/ha or more, 125 g ai/ha or more, 130 g ai/ha or more, 135 g ai/ha or more, 140 g ai/ha or more, 145 g ai/ha or more, 150 g ai/ha or more, 155 g ai/ha or more, 160 g ai/ha or more, 165 g ai/ha or more, 170 g ai/ha or more, 175 g ai/ha or more, 180 g ai/ha or more, 185 g ai/ha or more, 190 g ai/ha or more, 195 g ai/ha or more, 200 g ai/ha or more, 210 g ai/ha or more, 220 g ai/ha or more, 230 g ai/ha or more, 240 g ai/ha or more, 250 g ai/ha or more, 260 g ai/ha or more, 270 g ai/ha or more, 280 g ai/ha or more, or 290 g ai/ha or more; in an amount of 300 g ai/ha or less (e.g., 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 195 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 145 g ai/ha or less, 140 g ai/ha or less, 135 g ai/ha or less, 130 g ai/ha or less, 125 g ai/ha or less, 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-300 g ai/ha., 60-275 g ai/ha, 85-250 g ai/ha, 60-220 g ai/ha, 120-280 g ai/ha, 150-180 g ai/ha, 70-160 g ai/ha, 55-240 g ai/ha, 90-290 g ai/ha, 75-100 g ai/ha, 115-270 g ai/ha, 70-200 g ai/ha, or 55-290 g ai/ha.

### Propaquizafop

Compositions and methods of the present disclosure can include propaquizafop. Propaquizafop, shown below, is an aryloxyphenoxypropionate that is to control a wide range of annual and perennial grasses in soybeans, cotton, sugar beets, potatoes, peanuts, peas, canola/oilseed rape and vegetables. Propaquizafop, as well as methods of preparing propaquizafop, are known in the art.

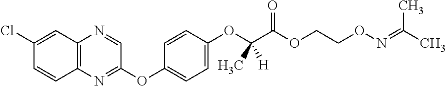 [SMILES: CC(C)=NOCCOC(=O)[C@@H](C)Oc1ccc(Oc2cnc3cc(Cl)ccc3n2)cc1]

Propaquizafop can be applied to vegetation or an area adjacent the vegetation or applied to soil or water to prevent the emergence or growth of vegetation in an amount sufficient to induce a herbicidal effect. In some aspects, propaquizafop is applied to vegetation or an area adjacent the vegetation or applied to soil or water to prevent the emergence or growth of vegetation in an amount of 29 g ai/ha or more, such as 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 210 g ai/ha or more, 220 g ai/ha or more, 230 g ai/ha or more, 240 g ai/ha or more, 250 g ai/ha or more, 260 g ai/ha or more, 270 g ai/ha or more, 280 g ai/ha or more, or 290 g ai/ha or more; in an amount of 300 g ai/ha or less (e.g., 290 g ai/ha or less, 280 g ai/ha or less, 270 g ai/ha or less, 260 g ai/ha or less, 250 g ai/ha or less, 240 g ai/ha or less, 230 g ai/ha or less, 220 g ai/ha or less, 210 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, or 30 g ai/ha or less; or in an amount ranging from any of the minimum values described above to any of the maximum values described above, such as 29-300 g ai/ha, 30-290 g ai/ha, 35-180 g ai/ha, 40-270 g ai/ha, 45-200 g ai/ha, 50-250 g ai/ha, 60-140 g ai/ha, 80-230 g ai/ha, 40-220 g ai/ha, 65-290 g ai/ha, 35-200 g ai/ha, 80-180 g ai/ha, 60-260 g ai/ha, 30-120 g ai/ha, 80-190 g ai/ha, 160-270 g ai/ha, or 30-290 g ai/ha.

### Quizalofop

Compositions and methods of the present disclosure can include quizalofop or an agriculturally acceptable salt or ester thereof. Quizalofop is an aryloxyphenoxypropionate that is used for selective post-emergence control of annual and perennial grass weeds in potatoes, soybeans, sugar beets, peanuts, canola/oilseed rape, sunflowers, vegetables, cotton and flax. Quizalofop-P-ethyl, shown below, is an ester of quizalofop. Quizalofop, as well as methods of preparing quizalofop, are known in the art.

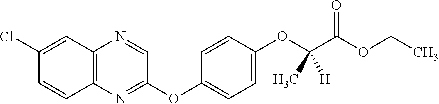 [SMILES: CCOC(=O)[C@@H](C)Oc1ccc(Oc2cnc3cc(Cl)ccc3n2)cc1]

Quizalofop can be applied to vegetation or an area adjacent the vegetation or applied to soil or water to prevent the emergence or growth of vegetation in an amount sufficient to induce a herbicidal effect. In some aspects, quizalofop is applied to vegetation or an area adjacent the vegetation or applied to soil or water to prevent the emergence or growth of vegetation in an amount of 24 g ai/ha or more, such as 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, or 245 g ai/ha or more; in an amount of 250 g ai/ha or less (e.g., 245 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 25 g ai/ha or less; or in an amount ranging from any of the minimum values described above to any of the maximum values described above, such as 24-250 g ai/ha, 25-225 g ai/ha, 55-200 g ai/ha, 40-190 g ai/ha, 25-80 g ai/ha, 60-175 g ai/ha, 110-225 g ai/ha, 75-160 g ai/ha, 45-220 g ai/ha, 80-150 g ai/ha, 30-90 g ai/ha, 40-100 g ai/ha, 50-180 g ai/ha, 60-230 g ai/ha, 60-120 g ai/ha, 40-100 g ai/ha, or 25-245 g ai/ha.

### Sethoxydim

Compositions and methods of the present disclosure can include sethoxydim or an agriculturally acceptable salt or ester thereof. Sethoxydim, shown below, is a cyclohexanedione herbicide that is used for controlling annual and perennial grasses in broad-leaved crops, including canola/oilseed rape, soybean, cotton, fodder beets, sunflowers, spinach, potatoes, tobacco, peanuts, strawberries, alfalfa, flax, vegetables, and sugar beet. Sethoxydim, as well as methods of preparing sethoxydim, are known in the art. Its herbicidal activity is described in The Pesticide Manual, Seventeenth Edition, 2016.

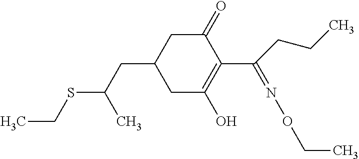 [SMILES: CCCC(=NOCC)C1=C(O)CC(CC(C)SCC)CC1=O]

Sethoxydim can be applied to vegetation or an area adjacent the vegetation or applied to soil or water to prevent the emergence or growth of vegetation in an amount sufficient to induce a herbicidal effect. In some aspects, sethoxydim is applied to vegetation or an area adjacent the vegetation or applied to soil or water to prevent the emergence or growth of vegetation in an amount of 2 g ai/ha or more, such as 3 g ai/ha or more, 4 g ai/ha or more, 5 g ai/ha or more, 6 g ai/ha or more, 8 g ai/ha or more, 10 g ai/ha or more, 12 g ai/ha or more, 14 g ai/ha or more, 16 g ai/ha or more, 18 g ai/ha or more, 20 g ai/ha or more, 22 g ai/ha or more, 24 g ai/ha or more, 26 g ai/ha or more, 28 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 80 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 620 g ai/ha or more, 640 g ai/ha or more, 660 g ai/ha or more, 680 g ai/ha or more, 700 g ai/ha or more, 750 g ai/ha or more, 800 g ai/ha or more, 850 g ai/ha or more, or 900 g ai/ha or more; in an amount of 900 g ai/ha or less, such as 850 g ai/ha or less, 800 g ai/ha or less, 750 g ai/ha or less, 700 g ai/ha or less, 680 g ai/ha or less, 660 g ai/ha or less, 640 g ai/ha or less, 620 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 290 g ai/ha or less, 280 g ai/ha or less, 270 g ai/ha or less, 260 g ai/ha or less, 250 g ai/ha or less, 240 g ai/ha or less, 235 g ai/ha or less, 230 g ai/ha or less, 225 g ai/ha or less, 220 g ai/ha or less, 215 g ai/ha or less, 210 g ai/ha or less, 205 g ai/ha or less, 200 g ai/ha or less, 195 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 80 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 28 g ai/ha or less, 26 g ai/ha or less, 24 g ai/ha or less, 22 g ai/ha or less, 20 g ai/ha or less, 18 g ai/ha or less, 16 g ai/ha or less, 14 g ai/ha or less, 12 g ai/ha or less, 10 g ai/ha or less, 8 g ai/ha or less, 6 g ai/ha or less, 5 g ai/ha or more, 4 g ai/ha or less, or 3 g ai/ha or less; or in an amount ranging from any of the minimum values described above to any of the maximum values described above, such as 2-900 g ai/ha, 3-500 g ai/ha, 5-270 g ai/ha, 10-600 g ai/ha, 22-440 g ai/ha, 35-560 g ai/ha, 10-400 g ai/ha, 20-700 g ai/ha, 40-280 g ai/ha, 60-680 g ai/ha, 100-750 g ai/ha, 90-300 g ai/ha, 200-700 g ai/ha, 300-500 g ai/ha, 3-100 g ai/ha, 5-90 g ai/ha, 12-105 g ai/ha, or 3-850 g ai/ha.

### Tepraloxydim

Compositions and methods of the present disclosure can include tepraloxydim or an agriculturally acceptable salt or ester thereof. Tepraloxydim, shown below, is a cyclohexanedione herbicide that is used for broad-spectrum post-emergence grass weed control, especially Poa annua and volunteer maize, and also Sorghum halepense and Elymus repens, in broad-leaved crops. Tepraloxydim, as well as methods of preparing tepraloxydim, are known in the art. Its herbicidal activity is described in The Pesticide Manual, Seventeenth Edition, 2016.

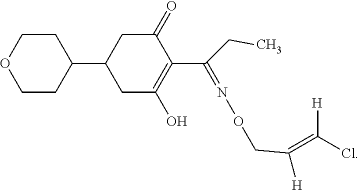 [SMILES: CCC(=NOC/C=C/Cl)C1=C(O)CC(C2CCOCC2)CC1=O]

Tepraloxydim can be applied to vegetation or an area adjacent the vegetation or applied to soil or water to prevent the emergence or growth of vegetation in an amount sufficient to induce a herbicidal effect. In some aspects, tepraloxydim is applied to vegetation or an area adjacent the vegetation or applied to soil or water to prevent the emergence or growth of vegetation in an amount of 25 g ai/ha or more, such as 27.5 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, or 195 g ai/ha or more; in an amount of 200 g ai/ha or less, such as 195 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, or 27.5 g ai/ha or less; or in an amount ranging from any of the minimum values described above to any of the maximum values described above, such as 25-200 g ai/ha, 25-190 g ai/ha, 60-180 g ai/ha, 35-170 g ai/ha, 45-120 g ai/ha, 25-65 g ai/ha, 90-160 g ai/ha, 75-195 g ai/ha, 25-150 g ai/ha, 30-190 g ai/ha, 35-130 g ai/ha, 50-120 g ai/ha, 65-180 g ai/ha, or 27.5-195 g ai/ha.

### Tralkoxydim

Compositions and methods of the present disclosure can include tralkoxydim or an agriculturally acceptable salt or ester thereof. Tralkoxydim is a cyclohexanedione herbicide that has the chemical name 2-[1-(ethoxyimino) propyl]-3-hydroxy-5-mesitylcyclohex-2-enone, and has the following structure:

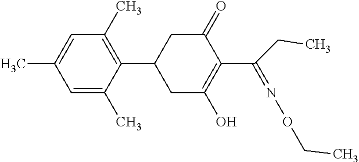 [SMILES: CCON=C(CC)C1=C(O)CC(c2c(C)cc(C)cc2C)CC1=O]

Tralkoxydim is used for post-emergence control of annual grass weeds (including Avena spp., Lolium spp., Setaria viridis, Phalaria spp., Alopecurus myosuroides, and Apera spica-venti) in wheat and barley. Tralkoxydim, as well as methods of preparing tralkoxydim, are known in the art. Its herbicidal activity is described in The Pesticide Manual, Seventeenth Edition, 2016.

Tralkoxydim can be applied to vegetation or an area adjacent the vegetation or applied to soil or water to prevent the emergence or growth of vegetation in an amount of 95 g ai/ha or more, such as 100 g ai/ha or more, 105 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 225 g ai/ha or more, 250 g ai/ha or more, 275 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, or 500 g ai/ha or more; in an amount of 501 g ai/ha or less, such as 500 g ai/ha or less, 475 g ai/ha or less, 450 g ai/ha or less, 425 g ai/ha or less, 400 g ai/ha or less, 375 g ai/ha or less, 350 g ai/ha or less, 325 g ai/ha or less, 300 g ai/ha or less, 275 g ai/ha or less, 250 g ai/ha or less, 225 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, or 100 g ai/ha or less; or in an amount ranging from any of the minimum values described above to any of the maximum values described above, such as 95-501 g ai/ha., 100-475 g ai/ha, 120-450 g ai/ha, 110-425 g ai/ha, 225-400 g ai/ha, 150-425 g ai/ha, 180-375 g ai/ha, 190-350 g ai/ha, 110-225 g ai/ha, 180-500 g ai/ha, or 100-500 g ai/ha.

### VLCFA Synthesis Inhibitor Herbicides

In addition to the compounds according to Formula I or agriculturally acceptable N-oxide, salt or ester thereof, the compositions include a very long chain fatty acid (VLCFA) synthesis inhibitor herbicide. Very long chain fatty acids have multiple functions in the plant, primarily serving as precursors of cuticle wax biosynthesis, and as components of storage lipids, sphingolipids and phospholipids. Examples of VLCFA synthesis inhibitors include, but are not limited to, acetochlor, alachlor, anilofos, butachlor, cafenstrole, dimethachlor, dimethenamid, diphenamid, fentrazamide, flufenacet, ipfencarbazone, mefenacet, metazachlor, metolachlor, naproanilide, napropamide, pethoxamid, piperophos, pretilachlor, propachlor, propisochlor, pyroxasulfone, and thenylchlor.

In some aspects, the composition can include a VLCFA synthesis inhibitor herbicide selected from the group consisting of acetochlor, alachlor, anilofos, butachlor, cafenstrole, dimethachlor, dimethenamid, diphenamid, fentrazamide, flufenacet, ipfencarbazone, mefenacet, metazachlor, metolachlor, naproanilide, napropamide, pethoxamid, piperophos, pretilachlor, propachlor, propisochlor, pyroxasulfone, thenylchlor, agriculturally acceptable salts and esters thereof, and combinations thereof.

The VLCFA synthesis inhibitor herbicide or agriculturally acceptable salt or ester thereof can be applied to vegetation or an area adjacent the vegetation or applied to soil or water to prevent the emergence or growth of vegetation in an amount sufficient to induce a herbicidal effect. In some aspects, the VLCFA synthesis inhibitor herbicide or agriculturally acceptable salt or ester thereof is applied to vegetation or an area adjacent the vegetation or applied to soil or water to prevent the emergence or growth of vegetation in an amount of 40 gram active ingredient per hectare (g ai/ha) or more, such as 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 250 g ai/ha or more, 260 g ai/ha or more, 280 g ai/ha or more, 300 g ai/ha or more, 320 g ai/ha or more, 340 g ai/ha or more, 350 g ai/ha or more, 360 g ai/ha or more, 380 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2050 g ai/ha or more, 2100 g ai/ha or more, 2150 g ai/ha or more, 2200 g ai/ha or more, 2240 g ai/ha or more, 2250 g ai/ha or more, 2300 g ai/ha or more, 2350 g ai/ha or more, 2400 g ai/ha or more, 2450 g ai/ha or more, 2500 g ai/ha or more, 2600 g ai/ha or more, 2700 g ai/ha or more, 2750 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3900 g ai/ha or more, 3950 g ai/ha or more, 4000 g ai/ha or more, 4100 g ai/ha or more, 4200 g ai/ha or more, 4250 g ai/ha or more, 4300 g ai/ha or more, 4400 g ai/ha or more, 4450 g ai/ha or more, 4500 g ai/ha or more, 4640 g ai/ha or more, 4780 g ai/ha or more, 4920 g ai/ha or more, 5060 g ai/ha or more, 5200 g ai/ha or more, 5340 g ai/ha or more, 5480 g ai/ha or more, 5620 g ai/ha or more, 5760 g ai/ha or more, 5900 g ai/ha or more, 6040 g ai/ha or more, 6180 g ai/ha or more, 6320 g ai/ha or more, 6460 g ai/ha or more, or 6600 g ai/ha or more; in an amount of 6720 g ai/ha or less, such as 6645 g ai/ha or less, 6575 g ai/ha or less, 6500 g ai/ha or less, 6425 g ai/ha or less, 6350 g ai/ha or less, 6275 g ai/ha or less, 6200 g ai/ha or less, 6125 g ai/ha or less, 6050 g ai/ha or less, 5975 g ai/ha or less, 5900 g ai/ha or less, 5825 g ai/ha or less, 5750 g ai/ha or less, 5675 g ai/ha or less, 5600 g ai/ha or less, 5525 g ai/ha or less, 5450 g ai/ha or less, 5375 g ai/ha or less, 5300 g ai/ha or less, 5225 g ai/ha or less, 5150 g ai/ha or less, 5075 g ai/ha or less, 5000 g ai/ha or less, 4925 g ai/ha or less, 4850 g ai/ha or less, 4775 g ai/ha or less, 4700 g ai/ha or less, 4625 g ai/ha or less, 4550 g ai/ha or less, 4475 g ai/ha or less, 4450 g ai/ha or less, 4400 g ai/ha or less, 4300 g ai/ha or less, 4250 g ai/ha or less, 4100 g ai/ha or less, 4000 g ai/ha or less, 3950 g ai/ha or less, 3900 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2750 g ai/ha or less, 2700 g ai/ha or less, 2600 g ai/ha or less, 2500 g ai/ha or less, 2450 g ai/ha or less, 2400 g ai/ha or less, 2350 g ai/ha or less, 2300 g ai/ha or less, 2250 g ai/ha or less, 2240 g ai/ha or less, 2200 g ai/ha or less, 2150 g ai/ha or less, 2100 g ai/ha or less, 2050 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1240 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 380 g ai/ha or less, 360 g ai/ha or less, 350 g ai/ha or less, 340 g ai/ha or less, 320 g ai/ha or less, 300 g ai/ha or less, 280 g ai/ha or less, 260 g ai/ha or less, 250 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, or 45 g ai/ha or less; or in an amount ranging from any of the minimum values described above to any of the maximum values described above, such as 40-6720 g ai/ha, 45-5825 g ai/ha, 60-2500 g ai/ha, 75-3100 g ai/ha, 90-900 g ai/ha, 55-4200 g ai/ha, 50-6050 g ai/ha, 80-4700 g ai/ha, 120-4775 g ai/ha, 65-2100 g ai/ha, 300-4000 g ai/ha, 1200-3600 g ai/ha, 250-5000 g ai/ha, 250-1000 g ai/ha, 700-4250 g ai/ha, 800-1400 g ai/ha, or 1000-6720 g ai/ha.

In some aspects, the composition contains: compounds according to Formula I; and (b) acetochlor, alachlor, anilofos, butachlor, cafenstrole, dimethachlor, dimethenamid, diphenamid, fentrazamide, flufenacet, ipfencarbazone, mefenacet, metazachlor, metolachlor, naproanilide, napropamide, pethoxamid, piperophos, pretilachlor, propachlor, propisochlor, pyroxasulfone, thenylchlor, or combinations thereof.

### Acetochlor

In some aspects, the VLCFA synthesis inhibitor herbicide can comprise acetochlor or an agriculturally acceptable salt or ester thereof. Acetochlor, shown below, is 2-chloro-N-(ethoxymethyl)-N-(2-ethyl-6-methylphenyl) acetamide. Its herbicidal activity is exemplified in The Pesticide Manual, Seventeenth Edition, 2016. Exemplary uses of acetochlor include its use pre-emergence or pre-plant to control annual grasses, certain annual broadleaf weeds and yellow nutsedge (Cyperus esculentus) in maize, peanuts, soybeans, cotton, potatoes and sugarcane.

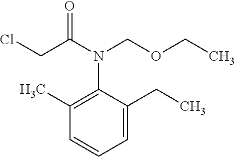 [SMILES: CCOCN(C(=O)CCl)c1c(C)cccc1CC]

Acetochlor can be applied to vegetation or an area adjacent the vegetation or applied to soil or water to prevent the emergence or growth of vegetation in an amount sufficient to induce a herbicidal effect. In some aspects, acetochlor is applied to vegetation or an area adjacent the vegetation or applied to soil or water to prevent the emergence or growth of vegetation in an amount of 2500 g ai/ha or more, such as 2600 g ai/ha or more, 2700 g ai/ha or more, 2750 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3900 g ai/ha or more, 3950 g ai/ha or more, 4000 g ai/ha or more, 4100 g ai/ha or more, 4200 g ai/ha or more, 4250 g ai/ha or more, 4300 g ai/ha or more, 4400 g ai/ha or more, 4450 g ai/ha or more, 4500 g ai/ha or more, 4640 g ai/ha or more, 4780 g ai/ha or more, 4920 g ai/ha or more, 5060 g ai/ha or more, 5200 g ai/ha or more, 5340 g ai/ha or more, 5480 g ai/ha or more, 5620 g ai/ha or more, 5760 g ai/ha or more, 5900 g ai/ha or more, 6040 g ai/ha or more, 6180 g ai/ha or more, 6320 g ai/ha or more, 6460 g ai/ha or more, or 6600 g ai/ha or more; in an amount of 6720 g ai/ha or less, such as 6645 g ai/ha or less, 6575 g ai/ha or less, 6500 g ai/ha or less, 6425 g ai/ha or less, 6350 g ai/ha or less, 6275 g ai/ha or less, 6200 g ai/ha or less, 6125 g ai/ha or less, 6050 g ai/ha or less, 5975 g ai/ha or less, 5900 g ai/ha or less, 5825 g ai/ha or less, 5750 g ai/ha or less, 5675 g ai/ha or less, 5600 g ai/ha or less, 5525 g ai/ha or less, 5450 g ai/ha or less, 5375 g ai/ha or less, 5300 g ai/ha or less, 5225 g ai/ha or less, 5150 g ai/ha or less, 5075 g ai/ha or less, 5000 g ai/ha or less, 4925 g ai/ha or less, 4850 g ai/ha or less, 4775 g ai/ha or less, 4700 g ai/ha or less, 4625 g ai/ha or less, 4550 g ai/ha or less, 4475 g ai/ha or less, 4450 g ai/ha or less, 4400 g ai/ha or less, 4300 g ai/ha or less, 4250 g ai/ha or less, 4100 g ai/ha or less, 4000 g ai/ha or less, 3950 g ai/ha or less, 3900 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2750 g ai/ha or less, 2700 g ai/ha or less, or 2600 g ai/ha or less; or in an amount ranging from any of the minimum values described above to any of the maximum values described above, such as 2500-6720 g ai/ha, 3000-5825 g ai/ha, 2700-5525 g ai/ha, 2800-4100 g ai/ha, 4775-6200 g ai/ha, 5000-6645 g ai/ha, 5150-6645 g ai/ha, or 4000-6720 g ai/ha.

### Alachlor

In some aspects, the VLCFA synthesis inhibitor herbicide can comprise alachlor or an agriculturally acceptable salt or ester thereof. Alachlor, show below, is 2-chloro-N-(2,6-diethylphenyl)-N-(methoxymethyl) acetamide. Its herbicidal activity is exemplified in The Pesticide Manual, Seventeenth Edition, 2016. Exemplary uses of alachlor include its use for pre-emergence control of annual grasses and many broadleaf weeds in cotton, brassicas, maize, oilseed rape, peanuts, radish, soybeans and sugar cane.

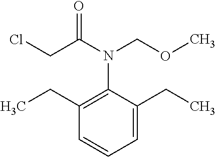 [SMILES: CCc1cccc(CC)c1N(COC)C(=O)CCl]

Alachlor can be applied to vegetation or an area adjacent the vegetation or applied to soil or water to prevent the emergence or growth of vegetation in an amount sufficient to induce a herbicidal effect. In some aspects, alachlor is applied to vegetation or an area adjacent the vegetation or applied to soil or water to prevent the emergence or growth of vegetation in an amount of 1500 g ai/ha or more, such as 1550 g ai/ha or more, 1600 g ai/ha or more, 1700 g ai/ha or more, 1800 g ai/ha or more, 1900 g ai/ha or more, 2000 g ai/ha or more, 2050 g ai/ha or more, 2100 g ai/ha or more, 2150 g ai/ha or more, 2200 g ai/ha or more, 2240 g ai/ha or more, 2250 g ai/ha or more, 2300 g ai/ha or more, 2350 g ai/ha or more, 2400 g ai/ha or more, 2450 g ai/ha or more, 2500 g ai/ha or more, 2600 g ai/ha or more, 2700 g ai/ha or more, 2750 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3900 g ai/ha or more, 3950 g ai/ha or more, 4000 g ai/ha or more, 4100 g ai/ha or more, 4200 g ai/ha or more, 4250 g ai/ha or more, 4300 g ai/ha or more, 4400 g ai/ha or more, or 4450 g ai/ha or more; in an amount of 4500 g ai/ha or less, such as 4475 g ai/ha or less, 4450 g ai/ha or less, 4400 g ai/ha or less, 4300 g ai/ha or less, 4250 g ai/ha or less, 4100 g ai/ha or less, 4000 g ai/ha or less, 3950 g ai/ha or less, 3900 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2750 g ai/ha or less, 2700 g ai/ha or less, 2600 g ai/ha or less, 2500 g ai/ha or less, 2450 g ai/ha or less, 2400 g ai/ha or less, 2350 g ai/ha or less, 2300 g ai/ha or less, 2250 g ai/ha or less, 2240 g ai/ha or less, 2200 g ai/ha or less, 2150 g ai/ha or less, 2100 g ai/ha or less, 2050 g ai/ha or less, 2000 g ai/ha or less, 1900 g ai/ha or less, 1800 g ai/ha or less, 1750 g ai/ha or less, 1700 g ai/ha or less, 1600 g ai/ha or less, or 1550 g ai/ha or less; or in an amount ranging from any of the minimum values described above to any of the maximum values described above, such as 1500-4500 g ai/ha, 1550-3900 g ai/ha, 2000-2500 g ai/ha, 2350-3700 g ai/ha, 3100-3900 g ai/ha, 2240-4200 g ai/ha, 2450-4300 g ai/ha, 3250-4475 g ai/ha, or 1900-4000 g ai/ha.

### Anilofos

In some aspects, the VLCFA synthesis inhibitor herbicide can comprise anilofos or an agriculturally acceptable salt or ester thereof. Anilofos, shown below, is S-[2-[(4-chlorophenyl) (1-methylethyl)amino]-2-oxoethyl] O,O-dimethyl phosphorodithioate. Its herbicidal activity is exemplified in The Pesticide Manual, Seventeenth Edition, 2016.

Exemplary uses of anilofos include its use for control of annual grassy weeds and sedges in transplanted rice.

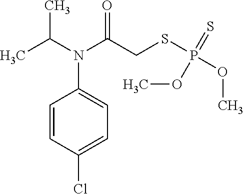 [SMILES: COP(=S)(OC)SCC(=O)N(c1ccc(Cl)cc1)C(C)C]

Anilofos can be applied to vegetation or an area adjacent the vegetation or applied to soil or water to prevent the emergence or growth of vegetation in an amount sufficient to induce a herbicidal effect. In some aspects, anilofos is applied to vegetation or an area adjacent the vegetation or applied to soil or water to prevent the emergence or growth of vegetation in an amount of 250 g ai/ha or more, such as 260 g ai/ha or more, 280 g ai/ha or more, 290 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1050 g ai/ha or more, 1100 g ai/ha or more, 1150 g ai/ha or more, 1200 g ai/ha or more, or 1250 g ai/ha or more; in an amount of 1300 g ai/ha or less, such as 1250 g ai/ha or less, 1200 g ai/ha or less, 1150 g ai/ha or less, 1100 g ai/ha or less, 1050 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 480 g ai/ha or less, 460 g ai/ha or less, 450 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 290 g ai/ha or less, 280 g ai/ha or less, or 260 g ai/ha or less; or in an amount ranging from any of the minimum values described above to any of the maximum values described above, such as 250-1300 g ai/ha, 260-1100 g ai/ha, 400-900 g ai/ha, 240-850 g ai/ha, 550-1000 g ai/ha, 400-775 g ai/ha, 320-1200 g ai/ha, 360-800 g ai/ha, 500-725 g ai/ha, 675-925 g ai/ha, 480-850 g ai/ha, 450-1050 g ai/ha, or 260-1250 g ai/ha.

### Butachlor

In some aspects, the VLCFA synthesis inhibitor herbicide can comprise butachlor or an agriculturally acceptable salt or ester thereof. Butachlor, shown below, is N-(butoxymethyl)-2-chloro-N-(2,6-diethylphenyl) acetamide. Its herbicidal activity is exemplified in The Pesticide Manual, Seventeenth Edition, 2016. Exemplary uses of butachlor include its use for pre-emergence control of annual grasses and certain broadleaf weeds in seeded and transplanted rice, barley, sugar beets, wheat, and brassicas.

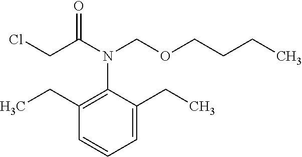 [SMILES: CCCCOCN(C(=O)CCl)c1c(CC)cccc1CC]

Butachlor thereof can be applied to vegetation or an area adjacent the vegetation or applied to soil or water to prevent the emergence or growth of vegetation in an amount sufficient to induce a herbicidal effect. In some aspects, butachlor is applied to vegetation or an area adjacent the vegetation or applied to soil or water to prevent the emergence or growth of vegetation in an amount of 400 g ai/ha or more, such as 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2050 g ai/ha or more, 2100 g ai/ha or more, 2150 g ai/ha or more, 2200 g ai/ha or more, 2240 g ai/ha or more, 2250 g ai/ha or more, 2300 g ai/ha or more, 2350 g ai/ha or more, 2400 g ai/ha or more, 2450 g ai/ha or more, 2500 g ai/ha or more, 2600 g ai/ha or more, 2700 g ai/ha or more, 2750 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3900 g ai/ha or more, 3950 g ai/ha or more, 4000 g ai/ha or more, 4100 g ai/ha or more, 4200 g ai/ha or more, 4250 g ai/ha or more, 4300 g ai/ha or more, 4400 g ai/ha or more, or 4450 g ai/ha or more; in an amount of 4500 g ai/ha or less, such as 4475 g ai/ha or less, 4450 g ai/ha or less, 4400 g ai/ha or less, 4300 g ai/ha or less, 4250 g ai/ha or less, 4100 g ai/ha or less, 4000 g ai/ha or less, 3950 g ai/ha or less, 3900 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2750 g ai/ha or less, 2700 g ai/ha or less, 2600 g ai/ha or less, 2500 g ai/ha or less, 2450 g ai/ha or less, 2400 g ai/ha or less, 2350 g ai/ha or less, 2300 g ai/ha or less, 2250 g ai/ha or less, 2240 g ai/ha or less, 2200 g ai/ha or less, 2150 g ai/ha or less, 2100 g ai/ha or less, 2050 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1240 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 740 g ai/ha or less, 700 g ai/ha or less, 650 g ai/ha or less, 600 g ai/ha or less, 550 g ai/ha or less, 500 g ai/ha or less, or 450 g ai/ha or less; or in an amount ranging from any of the minimum values described above to any of the maximum values described above, such as 400-4500 g ai/ha, 750-1800 g ai/ha, 1200-2900 g ai/ha, 1500-3100 g ai/ha, 2350-3900 g ai/ha, 500-1250 g ai/ha, 2000-3600 g ai/ha, 2750-4000 g ai/ha, or 450-4475 g ai/ha.

### Cafenstrole

In some aspects, the VLCFA synthesis inhibitor herbicide can comprise cafenstrole or an agriculturally acceptable salt or ester thereof. Cafenstrole, shown below, is N,N-diethyl-3-[(2,4,6-trimethylphenyl)sulfonyl]-1H-1,2,4-triazole-1-carboxamide. Its herbicidal activity is exemplified in The Pesticide Manual, Seventeenth Edition, 2016. Exemplary uses of cafenstrole include its use for pre- and post-emergence control of Echinochloa oryzicola, Cyperus difformis and other weeds in paddy rice.

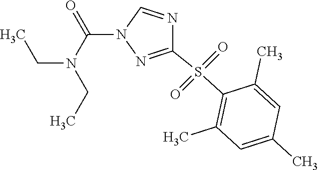 [SMILES: CCN(CC)C(=O)n1cnc(S(=O)(=O)c2c(C)cc(C)cc2C)n1]

Cafenstrole can be applied to vegetation or an area adjacent the vegetation or applied to soil or water to prevent the emergence or growth of vegetation in an amount sufficient to induce a herbicidal effect. In some aspects, cafenstrole is applied to vegetation or an area adjacent the vegetation or applied to soil or water to prevent the emergence or growth of vegetation in an amount of 50 g ai/ha or more, such as 60 g ai/ha or more, 75 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 250 g ai/ha or more, 260 g ai/ha or more, 280 g ai/ha or more, 300 g ai/ha or more, 320 g ai/ha or more, 340 g ai/ha or more, 350 g ai/ha or more, 360 g ai/ha or more, or 380 g ai/ha or more; in an amount of 400 g ai/ha or less, such as 380 g ai/ha or less, 360 g ai/ha or less, 350 g ai/ha or less, 340 g ai/ha or less, 320 g ai/ha or less, 300 g ai/ha or less, 280 g ai/ha or less, 260 g ai/ha or less, 250 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 75 g ai/ha or less, or 60 g ai/ha or less; or in an amount ranging from any of the minimum values described above to any of the maximum values described above, such as 50-400 g ai/ha, 120-380 g ai/ha, 90-260 g ai/ha, 130-300 g ai/ha, 250-360 g ai/ha, or 110-400 g ai/ha.

### Dimethachlor

In some aspects, the VLCFA synthesis inhibitor herbicide can comprise dimethachlor or an agriculturally acceptable salt or ester thereof. Dimethachlor, shown below, is 2-chloro-N-(2,6-dimethylphenyl)-N-(2-methoxyethyl) acetamide. Its herbicidal activity is exemplified in The Pesticide Manual, Seventeenth Edition, 2016. Exemplary uses of dimethachlor include its use for pre-emergence control of most annual grass (Alopecurus myosuroides, Apera spica-venti and Poa annua) and broadleaf weeds in oilseed rape.

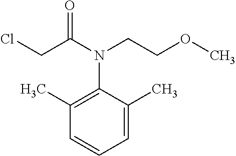 [SMILES: COCCN(C(=O)CCl)c1c(C)cccc1C]

Dimethachlor can be applied to vegetation or an area adjacent the vegetation or applied to soil or water to prevent the emergence or growth of vegetation in an amount sufficient to induce a herbicidal effect. In some aspects, dimethachlor is applied to vegetation or an area adjacent the vegetation or applied to soil or water to prevent the emergence or growth of vegetation in an amount of 1250 g ai/ha or more, such as 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, or 1950 g ai/ha or more; in an amount of 2000 g ai/ha or less, such as 1950 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, or 1300 g ai/ha or less; or in an amount ranging from any of the minimum values described above to any of the maximum values described above, such as 1250-2000 g ai/ha, 1400-1950 g ai/ha, 1300-1900 g ai/ha, 1250-1500 g ai/ha, 1600-1950 g ai/ha, 1700-2000 g ai/ha, or 1500-1750 g ai/ha.

### Dimethenamid

In some aspects, the VLCFA synthesis inhibitor herbicide can comprise dimethenamid or an agriculturally acceptable salt or ester thereof. Dimethenamid, shown below, is (RS)-2-chloro-N-(2,4-dimethyl-3-thienyl)-N-(2-methoxy-1-methylethyl) acetamide. Herbicidal activity for dimethenamid is exemplified in The Pesticide Manual, Seventeenth Edition, 2016. Exemplary uses of dimethenamid include its use for pre- or early post-emergence control of annual grass and broadleaf weeds in maize, soybeans, sugar beet, potatoes, dry beans and other crops.

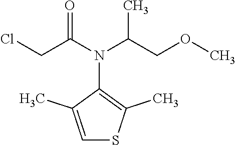 [SMILES: COCC(C)N(C(=O)CCl)c1c(C)csc1C]

Dimethenamid-P, (S)-2-chloro-N-(2,4-dimethyl-3-thienyl)-N-(2-methoxy-1-methyl-ethyl) acetamide, is a herbicidally active enantiomer of dimethenamid.

Dimethenamid can be applied to vegetation or an area adjacent the vegetation or applied to soil or water to prevent the emergence or growth of vegetation in an amount sufficient to induce a herbicidal effect. In some aspects, dimethenamid is applied to vegetation or an area adjacent the vegetation or applied to soil or water to prevent the emergence or growth of vegetation in an amount of 400 g ai/ha or more, such as 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1440 g ai/ha or more, or 1450 g ai/ha or more; in an amount of 1500 g ai/ha or less, such as 1450 g ai/ha or less, 1440 g ai/ha or less, 1400 g ai/ha or less, 1350 g ai/ha or less, 1300 g ai/ha or less, 1250 g ai/ha or less, 1240 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, or 450 g ai/ha or less; or in an amount ranging from any of the minimum values described above to any of the maximum values described above, such as 400-1500 g ai/ha, 550-1350 g ai/ha, 450-900 g ai/ha, 700-1400 g ai/ha, 1200-1440 g ai/ha, 650-1500 g ai/ha, 1050-1250 g ai/ha, 800-1350 g ai/ha, or 450-1450 g ai/ha.

### Diphenamid

In some aspects, the VLCFA synthesis inhibitor herbicide can comprise diphenamid or an agriculturally acceptable salt or ester thereof. Diphenamid, shown below, is N,N-dimethyl-α-phenylbenzeneacetamide. Its herbicidal activity is exemplified in The Pesticide Manual, Seventeenth Edition, 2016. Exemplary uses of diphenamid include its use for pre-emergence control of annual grasses and broadleaf weeds in a wide range of crops, including cotton, potatoes, sweet potatoes, tomatoes, vegetables, capsicums, okra, soybeans, peanuts, tobacco, orchard fruit, bush fruit, strawberries, forestry nurseries, and ornamental plants, shrubs and trees.

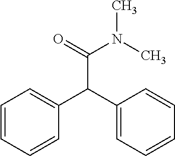 [SMILES: CN(C)C(=O)C(c1ccccc1)c1ccccc1]

Diphenamid can be applied to vegetation or an area adjacent the vegetation or applied to soil or water to prevent the emergence or growth of vegetation in an amount sufficient to induce a herbicidal effect. In some aspects, diphenamid is applied to vegetation or an area adjacent the vegetation or applied to soil or water to prevent the emergence or growth of vegetation in an amount of 3500 g ai/ha or more, such as 3600 g ai/ha or more, 3700 g ai/ha or more, 3750 g ai/ha or more, 3800 g ai/ha or more, 3900 g ai/ha or more, 3950 g ai/ha or more, 4000 g ai/ha or more, 4100 g ai/ha or more, 4200 g ai/ha or more, 4250 g ai/ha or more, 4300 g ai/ha or more, 4400 g ai/ha or more, 4500 g ai/ha or more, 4600 g ai/ha or more, 4700 g ai/ha or more, 4750 g ai/ha or more, 4800 g ai/ha or more, 4900 g ai/ha or more, 5000 g ai/ha or more, 5100 g ai/ha or more, 5200 g ai/ha or more, 5250 g ai/ha or more, 5300 g ai/ha or more, 5400 g ai/ha or more, 5500 g ai/ha or more, 5600 g ai/ha or more, 5700 g ai/ha or more, 5750 g ai/ha or more, 5800 g ai/ha or more, 5900 g ai/ha or more, or 5950 g ai/ha or more; in an amount of 6000 g ai/ha or less, such as 5950 g ai/ha or less, 5900 g ai/ha or less, 5800 g ai/ha or less, 5750 g ai/ha or less, 5700 g ai/ha or less, 5600 g ai/ha or less, 5500 g ai/ha or less, 5400 g ai/ha or less, 5300 g ai/ha or less, 5250 g ai/ha or less, 5200 g ai/ha or less, 5100 g ai/ha or less, 5000 g ai/ha or less, 4900 g ai/ha or less, 4800 g ai/ha or less, 4750 g ai/ha or less, 4700 g ai/ha or less, 4600 g ai/ha or less, 4500 g ai/ha or less, 4400 g ai/ha or less, 4300 g ai/ha or less, 4250 g ai/ha or less, 4200 g ai/ha or less, 4100 g ai/ha or less, 4000 g ai/ha or less, 3950 g ai/ha or less, 3900 g ai/ha or less, 3800 g ai/ha or less, 3750 g ai/ha or less, 3700 g ai/ha or less, or 3600 g ai/ha or less; or in an amount ranging from any of the minimum values described above to any of the maximum values described above, such as 3500-6000 g ai/ha, 4100-5800 g ai/ha, 3750-5500 g ai/ha, 5000-6000 g ai/ha, 5250-5950 g ai/ha, 3600-4750 g ai/ha, 3700-4250 g ai/ha, 4800-5400 g ai/ha, or 3900-5900 g ai/ha.

### Fenoxasulfone

In some aspects, the VLCFA synthesis inhibitor herbicide can comprise fenoxasulfone or an agriculturally acceptable salt or ester thereof. Fenoxasulfone, shown below, is 2,5-dichloro-4-ethoxybenzyl-4,5-dihydro-5,5-dimethyl-1,2-oxazol-3-yl sulfone. Its herbicidal use is reported in the online version of The Pesticide Manual, Seventeenth Edition, 2016. Exemplary uses of fenoxasulfone include control of grass and broadleaf weeds in rice.

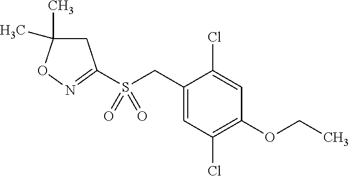 [SMILES: CCOc1cc(Cl)c(CS(=O)(=O)C2=NOC(C)(C)C2)cc1Cl]

### Fentrazamide

In some aspects, the VLCFA synthesis inhibitor herbicide can comprise fentrazamide or an agriculturally acceptable salt or ester thereof. Fentrazamide, shown below, is 4-(2-chlorophenyl)-N-cyclohexyl-N-ethyl-4,5-dihydro-5-oxo-1H-tetrazole-1-carboxamide. Its herbicidal activity is exemplified in The Pesticide Manual, Seventeenth Edition, 2016. Exemplary uses of fentrazamide include its use for pre- and early post-emergence control of barnyard grass (Echinochloa spp.) and annual sedges in rice.

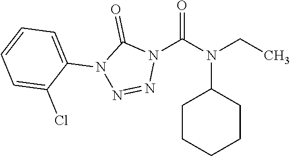 [SMILES: CCN(C(=O)n1nnn(-c2ccccc2Cl)c1=O)C1CCCCC1]

### Flufenacet

In some aspects, the VLCFA synthesis inhibitor herbicide can comprise flufenacet or an agriculturally acceptable salt or ester thereof. Flufenacet, shown below, is N-(4-fluorophenyl)-N-(1-methylethyl)-2-[[5-(trifluoromethyl)-1,3,4-thiadiazol-2-yl]oxy]acetamide. Its herbicidal activity is exemplified in The Pesticide Manual, Seventeenth Edition, 2016. Exemplary uses of flufenacet include its use for control of grass and broadleaf weeds in maize, wheat, rice, soybeans, tomatoes, potatoes, and sunflowers.

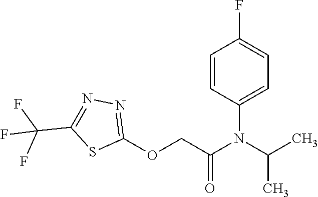 [SMILES: CC(C)N(C(=O)COc1nnc(C(F)(F)F)s1)c1ccc(F)cc1]

Flufenacet can be applied to vegetation or an area adjacent the vegetation or applied to soil or water to prevent the emergence or growth of vegetation in an amount sufficient to induce a herbicidal effect. In some aspects, flufenacet is applied to vegetation or an area adjacent the vegetation or applied to soil or water to prevent the emergence or growth of vegetation in an amount of 40 g ai/ha or more, such as 45 g ai/ha or more, 50 g ai/ha or more, 60 g ai/ha or more, 70 g ai/ha or more, 80 g ai/ha or more, 90 g ai/ha or more, 100 g ai/ha or more, 120 g ai/ha or more, 140 g ai/ha or more, 150 g ai/ha or more, 160 g ai/ha or more, 180 g ai/ha or more, 200 g ai/ha or more, 220 g ai/ha or more, 240 g ai/ha or more, 250 g ai/ha or more, 260 g ai/ha or more, 280 g ai/ha or more, 290 g ai/ha or more, 300 g ai/ha or more, 320 g ai/ha or more, 340 g ai/ha or more, 345 g ai/ha or more, 350 g ai/ha or more, 360 g ai/ha or more, 380 g ai/ha or more, 400 g ai/ha or more, 420 g ai/ha or more, 440 g ai/ha or more, 450 g ai/ha or more, 460 g ai/ha or more, 480 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 880 g ai/ha or more, or 890 g ai/ha or more; in an amount of 900 g ai/ha or less, such as 890 g ai/ha or less, 880 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 480 g ai/ha or less, 460 g ai/ha or less, 450 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 290 g ai/ha or less, 280 g ai/ha or less, 260 g ai/ha or less, 250 g ai/ha or less, 240 g ai/ha or less, 220 g ai/ha or less, 200 g ai/ha or less, 180 g ai/ha or less, 160 g ai/ha or less, 150 g ai/ha or less, 140 g ai/ha or less, 120 g ai/ha or less, 100 g ai/ha or less, 90 g ai/ha or less, 80 g ai/ha or less, 70 g ai/ha or less, 60 g ai/ha or less, 50 g ai/ha or less, 45 g ai/ha or less, or 42.5 g ai/ha or less; or in an amount ranging from any of the minimum values described above to any of the maximum values described above, such as 40-900 g ai/ha, 45-600 g ai/ha, 60-900 g ai/ha, 140-850 g ai/ha, 250-620 g ai/ha, 400-780 g ai/ha, 220-640 g ai/ha, 360-800 g ai/ha, 300-725 g ai/ha, 90-825 g ai/ha, 140-550 g ai/ha, 450-650 g ai/ha, or 50-880 g ai/ha.

### Indanofan

In some aspects, the VLCFA synthesis inhibitor herbicide can comprise indanofan or an agriculturally acceptable salt or ester thereof. Indanofan, shown below, is (RS)-2-[2-(3-chlorophenyl)-2,3-epoxypropyl]-2-ethylindan-1,3-dione. Its herbicidal activity is exemplified in the online edition of The Pesticide Manual. Exemplary uses of indanofan include pre- and post-emergence

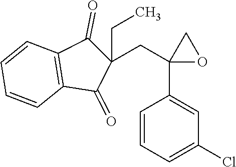 [SMILES: CCC1(CC2(c3cccc(Cl)c3)CO2)C(=O)c2ccccc2C1=O]

control of Echinochloa crus-galli, Monochoria vaginalis, Lindernia procumbens, Cyperus difformis, Scirpus juncoides, and Eleocharis acicularis in transplanted rice.

### Ipfencarbazone

In some aspects, the VLCFA synthesis inhibitor herbicide can comprise ipfencarbazone or an agriculturally acceptable salt or ester thereof. Ipfencarbazone, shown below, is 1-(2,4-dichlorophenyl)-N-(2,4-difluorophenyl)-1,5-dihydro-N-(1-methylethyl)-5-oxo-4H-1,2,4-triazole-4-carboxamide. Its herbicidal activity is exemplified in the online edition of The Pesticide Manual. Exemplary uses of ipfencarbazone include its use for pre- or early post-emergence control of grass, sedge, and some broadleaf weeds in rice.

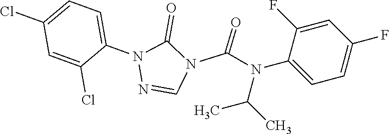 [SMILES: CC(C)N(C(=O)n1cnn(-c2ccc(Cl)cc2Cl)c1=O)c1ccc(F)cc1F]

Ipfencarbazone can be applied to vegetation or an area adjacent the vegetation or applied to soil or water to prevent the emergence or growth of vegetation in an amount sufficient to induce a herbicidal effect. In some aspects, ipfencarbazone is applied to vegetation or an area adjacent the vegetation or applied to soil or water to prevent the emergence or growth of vegetation in an amount of 150 g ai/ha or more, such as 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, or 390 g ai/ha or more; in an amount of 400 g ai/ha or less, such as 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, or 160 g ai/ha or less; or in an amount ranging from any of the minimum values described above to any of the maximum values described above, such as 150-400 g ai/ha, 175-380 g ai/ha, 210-275 g ai/ha, 225-310 g ai/ha, 300-390 g ai/ha, 280-400 g ai/ha, 200-350 g ai/ha, 160-325 g ai/ha, 250-300 g ai/ha, or 180-360 g ai/ha.

### Mefenacet

In some aspects, the VLCFA synthesis inhibitor herbicide can comprise mefenacet or an agriculturally acceptable salt or ester thereof. Mefenacet, shown below, is 2-(2-benzothiazolyloxy)-N-methyl-N-phenylacetamide. Its herbicidal activity is exemplified in The Pesticide Manual, Seventeenth Edition, 2016. Exemplary uses of mefenacet include its use for pre-emergence and early post-emergence control of grass weeds in transplanted rice.

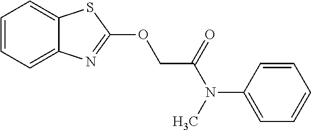 [SMILES: CN(C(=O)COc1nc2ccccc2s1)c1ccccc1]

Mefenacet can be applied to vegetation or an area adjacent the vegetation or applied to soil or water to prevent the emergence or growth of vegetation in an amount sufficient to induce a herbicidal effect. In some aspects, mefenacet is applied to vegetation or an area adjacent the vegetation or applied to soil or water to prevent the emergence or growth of vegetation in an amount of 40 g ai/ha or more, such as 50 g ai/ha or more, 60 g ai/ha or more, 70 g ai/ha or more, 80 g ai/ha or more, 90 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 250 g ai/ha or more, 300 g ai/ha or more, 350 g ai/ha or more, 400 g ai/ha or more, 450 g ai/ha or more, 500 g ai/ha or more, 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, or 1700 g ai/ha or more; in an amount of 1750 g ai/ha or less such as 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1240 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, 550 g ai/ha or less, 500 g ai/ha or less, 450 g ai/ha or less, 400 g ai/ha or less, 350 g ai/ha or less, 300 g ai/ha or less, 250 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 90 g ai/ha or less, 80 g ai/ha or less, 70 g ai/ha or less, 60 g ai/ha or less, or 50 g ai/ha or less; or in an amount ranging from any of the minimum values described above to any of the maximum values described above, such as 40-1750 g ai/ha, 150-1700 g ai/ha, 300-1500 g ai/ha, 850-1600 g ai/ha, 450-1240 g ai/ha, 1100-1400 g ai/ha, 800-1400 g ai/ha, or 1000-1750 g ai/ha.

### Metazachlor

In some aspects, the VLCFA synthesis inhibitor herbicide can comprise metazachlor or an agriculturally acceptable salt or ester thereof. Metazachlor, shown below, is 2-chloro-N-(2,6-dimethylphenyl)-N-(1H-pyrazol-1-ylmethyl) acetamide. Its herbicidal activity is exemplified in The Pesticide Manual, Seventeenth Edition, 2016. Exemplary uses of metazachlor include its use for pre- and early post-emergence control of winter and annual grasses and broadleaf weeds in a variety of vegetable, fruit, grain, and orchard crops.

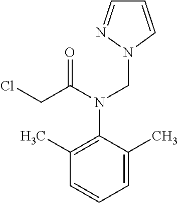 [SMILES: Cc1cccc(C)c1N(Cn1cccn1)C(=O)CCl]

Metazachlor can be applied to vegetation or an area adjacent the vegetation or applied to soil or water to prevent the emergence or growth of vegetation in an amount sufficient to induce a herbicidal effect. In some aspects, metazachlor is applied to vegetation or an area adjacent the vegetation or applied to soil or water to prevent the emergence or growth of vegetation in an amount of 750 g ai/ha or more, such as 775 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1440 g ai/ha or more, or 1450 g ai/ha or more; in an amount of 1500 g ai/ha or less, such as 1450 g ai/ha or less, 1440 g ai/ha or less, 1400 g ai/ha or less, 1350 g ai/ha or less, 1300 g ai/ha or less, 1250 g ai/ha or less, 1240 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, or 775 g ai/ha or less; or in an amount ranging from any of the minimum values described above to any of the maximum values described above, such as 750-1500 g ai/ha, 850-1350 g ai/ha, 800-900 g ai/ha, 950-1400 g ai/ha, 1200-1440 g ai/ha, 1250-1500 g ai/ha, 1050-1250 g ai/ha, 800-1350 g ai/ha, or 1000-1450 g ai/ha.

### Metolachlor

In some aspects, the VLCFA synthesis inhibitor herbicide can comprise metolachlor or an agriculturally acceptable salt or ester thereof. Metolachlor, shown below, is 2-chloro-N-(2-ethyl-6-methylphenyl)-N-(2-methoxy-1-methylethyl) acetamide. Its herbicidal activity is exemplified in The Pesticide Manual, Seventeenth Edition, 2016. Exemplary uses of metolachlor include its use for control of annual grasses and broadleaf weeds in maize, sorghum, cotton, sugar beet, fodder beet, sugarcane, potatoes, peanuts, soybeans, safflowers, sunflowers, various vegetables, fruit and nut trees, and woody ornamentals.

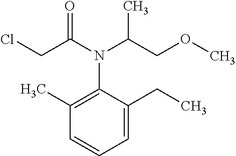 [SMILES: CCc1cccc(C)c1N(C(=O)CCl)C(C)COC]

S-metolachlor is a mixture of isomers, containing of 80-200% (S) to 20-0% (R) isomers of metolachlor.

Metolachlor can be applied to vegetation or an area adjacent the vegetation or applied to soil or water to prevent the emergence or growth of vegetation in an amount sufficient to induce a herbicidal effect. In some aspects, metolachlor is applied to vegetation or an area adjacent the vegetation or applied to soil or water to prevent the emergence or growth of vegetation in an amount of 500 g ai/ha or more, such as 550 g ai/ha or more, 600 g ai/ha or more, 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2050 g ai/ha or more, 2100 g ai/ha or more, 2150 g ai/ha or more, 2200 g ai/ha or more, 2240 g ai/ha or more, 2250 g ai/ha or more, 2300 g ai/ha or more, 2350 g ai/ha or more, 2400 g ai/ha or more, 2450 g ai/ha or more, 2500 g ai/ha or more, or 2550 g ai/ha or more; in an amount of 2600 g ai/ha or less, such as 2550 g ai/ha or less, 2500 g ai/ha or less, 2450 g ai/ha or less, 2400 g ai/ha or less, 2350 g ai/ha or less, 2300 g ai/ha or less, 2250 g ai/ha or less, 2240 g ai/ha or less, 2200 g ai/ha or less, 2150 g ai/ha or less, 2100 g ai/ha or less, 2050 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1240 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 650 g ai/ha or less, 600 g ai/ha or less, or 550 g ai/ha or less; or in an amount ranging from any of the minimum values described above to any of the maximum values described above, such as 500-2600 g ai/ha, 1500-2450 g ai/ha, 1240-2500 g ai/ha, 750-2000 g ai/ha, 1300-1900 g ai/ha, 800-1450 g ai/ha, 2000-2500 g ai/ha, 650-1600 g ai/ha, or 550-2550 g ai/ha.

### Naproanilide

In some aspects, the VLCFA synthesis inhibitor herbicide can comprise naproanilide or an agriculturally acceptable salt or ester thereof. Naproanilide, shown below, is 2-(2-naphthalenyloxy)-N-phenyl propanamide. Its herbicidal activity is exemplified in the online edition of The Pesticide Manual. Exemplary uses of naproanilide include its use for control of annual and some perennial weeds, but not Echinochloa crus-galli, in paddy rice, as well as good control of young Sagittaria pygmaea.

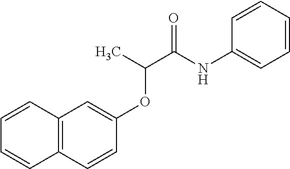 [SMILES: CC(Oc1ccc2ccccc2c1)C(=O)Nc1ccccc1]

Naproanilide can be applied to vegetation or an area adjacent the vegetation or applied to soil or water to prevent the emergence or growth of vegetation in an amount sufficient to induce a herbicidal effect. In some aspects, naproanilide is applied to vegetation or an area adjacent the vegetation or applied to soil or water to prevent the emergence or growth of vegetation in an amount of 2000 g ai/ha or more, such as 2050 g ai/ha or more, 2100 g ai/ha or more, 2150 g ai/ha or more, 2200 g ai/ha or more, 2240 g ai/ha or more, 2250 g ai/ha or more, 2300 g ai/ha or more, 2350 g ai/ha or more, 2400 g ai/ha or more, 2450 g ai/ha or more, 2500 g ai/ha or more, 2600 g ai/ha or more, 2700 g ai/ha or more, 2750 g ai/ha or more, 2800 g ai/ha or more, 2900 g ai/ha or more, or 2950 g ai/ha or more; in an amount of 3000 g ai/ha or less, such as 2950 g ai/ha or less, 2900 g ai/ha or less, 2800 g ai/ha or less, 2750 g ai/ha or less, 2700 g ai/ha or less, 2600 g ai/ha or less, 2500 g ai/ha or less, 2450 g ai/ha or less, 2400 g ai/ha or less, 2350 g ai/ha or less, 2300 g ai/ha or less, 2250 g ai/ha or less, 2240 g ai/ha or less, 2200 g ai/ha or less, 2150 g ai/ha or less, 2100 g ai/ha or less, or 2050 g ai/ha or less; or in an amount ranging from any of the minimum values described above to any of the maximum values described above, such as 2000-3000 g ai/ha, 2150-2750 g ai/ha, 2200-2500 g ai/ha, 2500-3000 g ai/ha, 2050-2600 g ai/ha, 2600-2950 g ai/ha, 2750-3000 g ai/ha, or 2240-2800 g ai/ha.

### Napropamide

In some aspects, the VLCFA synthesis inhibitor herbicide can comprise napropamide or an agriculturally acceptable salt or ester thereof. Napropamide, shown below, is (RS)-N,N-diethyl-2-(1-naphthyloxy) propionamide. Its herbicidal activity is exemplified in The Pesticide Manual, Seventeenth Edition, 2016. Exemplary uses of napropamide include its use for pre-emergence control of annual grasses and broadleaf weeds in asparagus, rhubarb, cucurbits, brassicas, oilseed rape, tomatoes, capsicums, potatoes, peas, nuts, fruit trees and bushes (including citrus), vines, strawberries, sunflowers, safflowers, ornamentals, tobacco, olives, figs, mint, turf, and other crops.

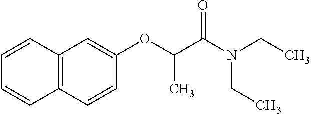 [SMILES: CCN(CC)C(=O)C(C)Oc1ccc2ccccc2c1]

Napropamide can be applied to vegetation or an area adjacent the vegetation or applied to soil or water to prevent the emergence or growth of vegetation in an amount sufficient to induce a herbicidal effect. In some aspects, napropamide is applied to vegetation or an area adjacent the vegetation or applied to soil or water to prevent the emergence or growth of vegetation in an amount of 2000 g ai/ha or more, such as 2050 g ai/ha or more, 2100 g ai/ha or more, 2150 g ai/ha or more, 2200 g ai/ha or more, 2240 g ai/ha or more, 2250 g ai/ha or more, 2300 g ai/ha or more, 2350 g ai/ha or more, 2400 g ai/ha or more, 2450 g ai/ha or more, 2500 g ai/ha or more, 2600 g ai/ha or more, 2700 g ai/ha or more, 2750 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3900 g ai/ha or more, 3950 g ai/ha or more, 4000 g ai/ha or more, 4100 g ai/ha or more, 4200 g ai/ha or more, 4250 g ai/ha or more, 4300 g ai/ha or more, 4400 g ai/ha or more, 4450 g ai/ha or more, 4500 g ai/ha or more, 4640 g ai/ha or more, 4780 g ai/ha or more, 4920 g ai/ha or more, 5060 g ai/ha or more, 5200 g ai/ha or more, 5340 g ai/ha or more, 5480 g ai/ha or more, 5620 g ai/ha or more, 5760 g ai/ha or more, 5825 g ai/ha or more, or 5900 g ai/ha or more; in an amount of 6000 g ai/ha or less, such as 5900 g ai/ha or less, 5825 g ai/ha or less, 5750 g ai/ha or less, 5675 g ai/ha or less, 5600 g ai/ha or less, 5525 g ai/ha or less, 5450 g ai/ha or less, 5375 g ai/ha or less, 5300 g ai/ha or less, 5225 g ai/ha or less, 5150 g ai/ha or less, 5075 g ai/ha or less, 5000 g ai/ha or less, 4925 g ai/ha or less, 4850 g ai/ha or less, 4775 g ai/ha or less, 4700 g ai/ha or less, 4625 g ai/ha or less, 4550 g ai/ha or less, 4475 g ai/ha or less, 4450 g ai/ha or less, 4400 g ai/ha or less, 4300 g ai/ha or less, 4250 g ai/ha or less, 4100 g ai/ha or less, 4000 g ai/ha or less, 3950 g ai/ha or less, 3900 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2750 g ai/ha or less, 2700 g ai/ha or less, 2600 g ai/ha or less, 2500 g ai/ha or less, 2450 g ai/ha or less, 2400 g ai/ha or less, 2350 g ai/ha or less, 2300 g ai/ha or less, 2250 g ai/ha or less, 2240 g ai/ha or less, 2200 g ai/ha or less, 2150 g ai/ha or less, 2100 g ai/ha or less, or 2050 g ai/ha or less; or in an amount ranging from any of the minimum values described above to any of the maximum values described above, such as 2000-6000 g ai/ha, 2150-5825 g ai/ha, 2800-5600 g ai/ha, 2350-3700 g ai/ha, 3250-5900 g ai/ha, 2400-4250 g ai/ha, 2500-5675 g ai/ha, 2700-4925 g ai/ha, or 2050-5525 g ai/ha.

### Pethoxamid

In some aspects, the VLCFA synthesis inhibitor herbicide can comprise pethoxamid or an agriculturally acceptable salt or ester thereof. Pethoxamid is 2-chloro-N-(2-ethoxyethyl)-N-(2-methyl-1-phenyl-1-propen-1-yl) acetamide. Its herbicidal activity is exemplified in The Pesticide Manual, Seventeenth Edition, 2016. Exemplary uses of pethoxamid include its use for pre- and early post-emergence control of grass weeds and broadleaf weeds in maize, oilseed rape and soybeans.

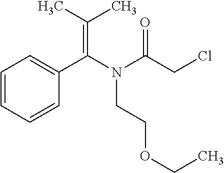 [SMILES: CCOCCN(C(=O)CCl)C(=C(C)C)c1ccccc1]

Pethoxamid can be applied to vegetation or an area adjacent the vegetation or applied to soil or water to prevent the emergence or growth of vegetation in an amount sufficient to induce a herbicidal effect. In some aspects, pethoxamid is applied to vegetation or an area adjacent the vegetation or applied to soil or water to prevent the emergence or growth of vegetation in an amount of 600 g ai/ha or more, such as 650 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, or 1475 g ai/ha or more; in an amount of 1500 g ai/ha or less, such as 1475 g ai/ha or less, 1450 g ai/ha or less, 1400 g ai/ha or less, 1350 g ai/ha or less, 1300 g ai/ha or less, 1250 g ai/ha or less, 1240 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, or 650 g ai/ha or less; or in an amount ranging from any of the minimum values described above to any of the maximum values described above, such as 600-1500 g ai/ha, 900-1400 g ai/ha, 1250-1450 g ai/ha, 800-1200 g ai/ha, 1350-1500 g ai/ha, 1000-1240 g ai/ha, or 650-1475 g ai/ha.

### Piperophos

In some aspects, the VLCFA synthesis inhibitor herbicide can comprise piperophos or an agriculturally acceptable salt or ester thereof. Piperophos, shown below, is S-[2-(2-methyl-1-piperidinyl)-2-oxoethyl] O,O-dipropylphosphorodithioate. Its herbicidal activity is exemplified in The Pesticide Manual, Seventeenth Edition, 2016. Exemplary uses of piperophos include its use as a selective herbicide active against annual grasses and sedges in direct seeded or transplanted rice.

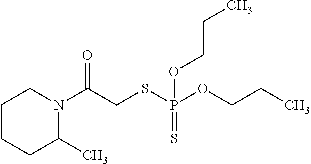 [SMILES: CCCOP(=S)(OCCC)SCC(=O)N1CCCCC1C]

Piperophos can be applied to vegetation or an area adjacent the vegetation or applied to soil or water to prevent the emergence or growth of vegetation in an amount sufficient to induce a herbicidal effect. In some aspects, piperophos is applied to vegetation or an area adjacent the vegetation or applied to soil or water to prevent the emergence or growth of vegetation in an amount of 300 g ai/ha or more, such as 310 g ai/ha or more, 320 g ai/ha or more, 330 g ai/ha or more, 340 g ai/ha or more, 350 g ai/ha or more, 360 g ai/ha or more, 370 g ai/ha or more, 380 g ai/ha or more, 390 g ai/ha or more, 400 g ai/ha or more, 410 g ai/ha or more, 420 g ai/ha or more, 430 g ai/ha or more, 440 g ai/ha or more, 450 g ai/ha or more, 460 g ai/ha or more, 480 g ai/ha or more, 500 g ai/ha or more, 520 g ai/ha or more, 540 g ai/ha or more, 550 g ai/ha or more, 560 g ai/ha or more, 580 g ai/ha or more, 600 g ai/ha or more, 620 g ai/ha or more, 640 g ai/ha or more, 650 g ai/ha or more, 660 g ai/ha or more, 680 g ai/ha or more, 700 g ai/ha or more, 720 g ai/ha or more, or 740 g ai/ha or more; in an amount of 750 g ai/ha or less, such as 740 g ai/ha or less, 720 g ai/ha or less, 700 g ai/ha or less, 680 g ai/ha or less, 660 g ai/ha or less, 650 g ai/ha or less, 640 g ai/ha or less, 620 g ai/ha or less, 600 g ai/ha or less, 580 g ai/ha or less, 560 g ai/ha or less, 550 g ai/ha or less, 540 g ai/ha or less, 520 g ai/ha or less, 500 g ai/ha or less, 480 g ai/ha or less, 460 g ai/ha or less, 450 g ai/ha or less, 440 g ai/ha or less, 430 g ai/ha or less, 420 g ai/ha or less, 410 g ai/ha or less, 400 g ai/ha or less, 390 g ai/ha or less, 380 g ai/ha or less, 370 g ai/ha or less, 360 g ai/ha or less, 350 g ai/ha or less, 340 g ai/ha or less, 330 g ai/ha or less, 320 g ai/ha or less, or 310 g ai/ha or less; or in an amount ranging from any of the minimum values described above to any of the maximum values described above, such as 300-750 g ai/ha, 320-580 g ai/ha, 330-660 g ai/ha, 410-650 g ai/ha, 340-700 g ai/ha, 450-520 g ai/ha, 500-750 g ai/ha, 400-600 g ai/ha, 350-720 g ai/ha, or 420-740 g ai/ha.

### Pretilachlor

In some aspects, the VLCFA synthesis inhibitor herbicide can comprise pretilachlor or an agriculturally acceptable salt or ester thereof. Pretilachlor, shown below, is 2-chloro-N-(2,6-diethylphenyl)-N-(2-propoxyethyl) acetamide. Its herbicidal activity is exemplified in The Pesticide Manual, Seventeenth Edition, 2016. Exemplary uses of pretilachlor include its use for control of annual grasses, broadleaf weeds and sedges in transplanted and seeded rice.

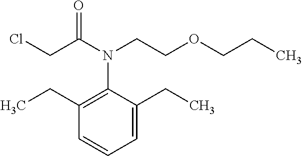 [SMILES: CCCOCCN(C(=O)CCl)c1c(CC)cccc1CC]

Pretilachlor can be applied to vegetation or an area adjacent the vegetation or applied to soil or water to prevent the emergence or growth of vegetation in an amount sufficient to induce a herbicidal effect. In some aspects, pretilachlor is applied to vegetation or an area adjacent the vegetation or applied to soil or water to prevent the emergence or growth of vegetation in an amount of 250 g ai/ha or more, such as 260 g ai/ha or more, 280 g ai/ha or more, 290 g ai/ha or more, 300 g ai/ha or more, 325 g ai/ha or more, 350 g ai/ha or more, 375 g ai/ha or more, 400 g ai/ha or more, 425 g ai/ha or more, 450 g ai/ha or more, 475 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 725 g ai/ha or more, 750 g ai/ha or more, 775 g ai/ha or more, 800 g ai/ha or more, 825 g ai/ha or more, 850 g ai/ha or more, 875 g ai/ha or more, 900 g ai/ha or more, 925 g ai/ha or more, 950 g ai/ha or more, 975 g ai/ha or more, 1000 g ai/ha or more, 1025 g ai/ha or more, 1050 g ai/ha or more, 1075 g ai/ha or more, 1100 g ai/ha or more, 1125 g ai/ha or more, 1150 g ai/ha or more, 1175 g ai/ha or more, 1200 g ai/ha or more, 1225 g ai/ha or more, 1250 g ai/ha or more, 1275 g ai/ha or more, 1300 g ai/ha or more, or 1325 g ai/ha or more; in an amount of 1350 g ai/ha or less, such as 1325 g ai/ha or less, 1300 g ai/ha or less, 1275 g ai/ha or less, 1250 g ai/ha or less, 1225 g ai/ha or less, 1200 g ai/ha or less, 1175 g ai/ha or less, 1150 g ai/ha or less, 1125 g ai/ha or less, 1100 g ai/ha or less, 1075 g ai/ha or less, 1050 g ai/ha or less, 1025 g ai/ha or less, 1000 g ai/ha or less, 975 g ai/ha or less, 950 g ai/ha or less, 925 g ai/ha or less, 900 g ai/ha or less, 875 g ai/ha or less, 850 g ai/ha or less, 825 g ai/ha or less, 800 g ai/ha or less, 775 g ai/ha or less, 750 g ai/ha or less, 725 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 480 g ai/ha or less, 460 g ai/ha or less, 450 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 290 g ai/ha or less, 280 g ai/ha or less, or 260 g ai/ha or less; or in an amount ranging from any of the minimum values described above to any of the maximum values described above, such as 250-1350 g ai/ha, 1025-1325 g ai/ha, 500-1250 g ai/ha, 440-850 g ai/ha, 1000-1300 g ai/ha, 300-1100 g ai/ha, or 260-1325 g ai/ha.

### Propachlor

In some aspects, the VLCFA synthesis inhibitor herbicide can comprise propachlor or an agriculturally acceptable salt or ester thereof. Propachlor, shown below, is 2-chloro-N-(1-methylethyl)-N-phenylacetamide. Its herbicidal activity is exemplified in The Pesticide Manual, Seventeenth Edition, 2016. It is used pre-emergence, pre-planting incorporated or early post-emergence control of annual grasses and some broadleaf weeds in beans, brassicas, cotton, sorghum, peanuts, leeks, maize, onions, peas, roses, ornamental trees and shrubs, soybeans and sugar cane.

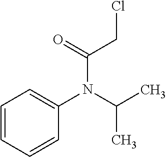

Propachlor can be applied to vegetation or an area adjacent the vegetation or applied to soil or water to prevent the emergence or growth of vegetation in an amount sufficient to induce a herbicidal effect. In some aspects, propachlor is applied to vegetation or an area adjacent the vegetation or applied to soil or water to prevent the emergence or growth of vegetation in an amount of 3250 g ai/ha or more, such as 3300 g ai/ha or more, 3360 g ai/ha or more, 3400 g ai/ha or more, 3500 g ai/ha or more, 3600 g ai/ha or more, 3700 g ai/ha or more, 3750 g ai/ha or more, 3800 g ai/ha or more, 3900 g ai/ha or more, 3950 g ai/ha or more, 4000 g ai/ha or more, 4100 g ai/ha or more, 4200 g ai/ha or more, 4250 g ai/ha or more, 4300 g ai/ha or more, 4400 g ai/ha or more, 4450 g ai/ha or more, 4500 g ai/ha or more, 4640 g ai/ha or more, 4780 g ai/ha or more, 4920 g ai/ha or more, 5060 g ai/ha or more, 5200 g ai/ha or more, 5340 g ai/ha or more, 5480 g ai/ha or more, 5620 g ai/ha or more, 5760 g ai/ha or more, 5900 g ai/ha or more, 6040 g ai/ha or more, 6180 g ai/ha or more, 6320 g ai/ha or more, 6460 g ai/ha or more, 6600 g ai/ha or more, or 6700 g ai/ha or more; in an amount of 6720 g ai/ha or less, such as 6700 g ai/ha or less, 6645 g ai/ha or less, 6600 g ai/ha or less, 6575 g ai/ha or less, 6500 g ai/ha or less, 6425 g ai/ha or less, 6350 g ai/ha or less, 6275 g ai/ha or less, 6200 g ai/ha or less, 6125 g ai/ha or less, 6050 g ai/ha or less, 5975 g ai/ha or less, 5900 g ai/ha or less, 5825 g ai/ha or less, 5750 g ai/ha or less, 5675 g ai/ha or less, 5600 g ai/ha or less, 5525 g ai/ha or less, 5450 g ai/ha or less, 5375 g ai/ha or less, 5300 g ai/ha or less, 5225 g ai/ha or less, 5150 g ai/ha or less, 5075 g ai/ha or less, 5000 g ai/ha or less, 4925 g ai/ha or less, 4850 g ai/ha or less, 4775 g ai/ha or less, 4700 g ai/ha or less, 4625 g ai/ha or less, 4550 g ai/ha or less, 4475 g ai/ha or less, 4450 g ai/ha or less, 4400 g ai/ha or less, 4300 g ai/ha or less, 4250 g ai/ha or less, 4100 g ai/ha or less, 4000 g ai/ha or less, 3950 g ai/ha or less, 3900 g ai/ha or less, 3800 g ai/ha or less, 3750 g ai/ha or less, 3700 g ai/ha or less, 3600 g ai/ha or less, 3500 g ai/ha or less, 3400 g ai/ha or less, 3360 g ai/ha or less, or 3300 g ai/ha or less; or in an amount ranging from any of the minimum values described above to any of the maximum values described above, such as 3250-6720 g ai/ha, 3500-5825 g ai/ha, 4250-6645 g ai/ha, 3950-6050 g ai/ha, 3300-4700 g ai/ha, 4100-6425 g ai/ha, or 3360-6720 g ai/ha.

### Propisochlor

In some aspects, the VLCFA synthesis inhibitor herbicide can comprise propisochlor or an agriculturally acceptable salt or ester thereof. Propisochlor, shown below, is 2-chloro-N-(2-ethyl-6-methylphenyl)-N-(isopropoxymethyl) acetamide. Its herbicidal activity is exemplified in The Pesticide Manual, Seventeenth Edition, 2016. Exemplary uses of propisochlor include its use in pre-plant or pre-emergence control of annual grass and some broadleaf weeds in maize, sunflowers, soybeans, potatoes, beans, sweet lupins, peas, and onions.

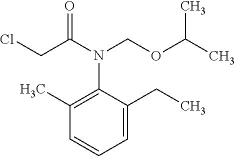 [SMILES: CCc1cccc(C)c1N(COC(C)C)C(=O)CCl]

Propisochlor can be applied to vegetation or an area adjacent the vegetation or applied to soil or water to prevent the emergence or growth of vegetation in an amount sufficient to induce a herbicidal effect. In some aspects, propisochlor is applied to vegetation or an area adjacent the vegetation or applied to soil or water to prevent the emergence or growth of vegetation in an amount of 900 g ai/ha or more, such as 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1550 g ai/ha or more, 1600 g ai/ha or more, 1650 g ai/ha or more, 1700 g ai/ha or more, 1750 g ai/ha or more, 1800 g ai/ha or more, 1850 g ai/ha or more, 1900 g ai/ha or more, or 1950 g ai/ha or more; in an amount of 2000 g ai/ha or less, such as 1950 g ai/ha or less, 1900 g ai/ha or less, 1850 g ai/ha or less, 1800 g ai/ha or less, 1750 g ai/ha or less, 1700 g ai/ha or less, 1650 g ai/ha or less, 1600 g ai/ha or less, 1550 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, or 950 g ai/ha or less; in an amount ranging from any of the minimum values described above to any of the maximum values described above, such as 900-2000 g ai/ha, 1050-1750 g ai/ha, 1200-1500 g ai/ha, 1400-1700 g ai/ha, 1100-1800 g ai/ha, 1600-1950 g ai/ha, 1350-1850 g ai/ha, 900-1550 g ai/ha, or 1000-1900 g ai/ha.

### Pyroxasulfone

In some aspects, the VLCFA synthesis inhibitor herbicide can comprise pyroxasulfone or an agriculturally acceptable salt or ester thereof. Pyroxasulfone, shown below, is 3-[[[5-(difluoromethoxy)-1-methyl-3-(trifluoromethyl)-1H-pyrazol-4-yl]methyl]sulfonyl]-4,5-dihydro-5,5-dimethylisoxazole. Its herbicidal activity is exemplified in The Pesticide Manual, Seventeenth Edition, 2016. Pyroxasulfone provides pre- and early post-emergence control of annual grasses and some broadleaf weeds in maize, soybeans, wheat, triticale, barley, and other crops.

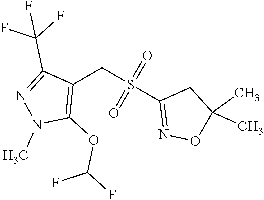 [SMILES: Cn1nc(C(F)(F)F)c(CS(=O)(=O)C2=NOC(C)(C)C2)c1OC(F)F]

The pyroxasulfone can be applied to vegetation or an area adjacent the vegetation or applied to soil or water to prevent the emergence or growth of vegetation in an amount sufficient to induce a herbicidal effect. In some aspects, the pyroxasulfone is applied to vegetation or an area adjacent the vegetation or applied to soil or water to prevent the emergence or growth of vegetation in an amount of 40 g ai/ha or more, such as 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 105 g ai/ha or more, 110 g ai/ha or more, 115 g ai/ha or more, 120 g ai/ha or more, 125 g ai/ha or more, 130 g ai/ha or more, 135 g ai/ha or more, 140 g ai/ha or more, 145 g ai/ha or more, 150 g ai/ha or more, 155 g ai/ha or more, 160 g ai/ha or more, 165 g ai/ha or more, 170 g ai/ha or more, 175 g ai/ha or more, 180 g ai/ha or more, 185 g ai/ha or more, 190 g ai/ha or more, 195 g ai/ha or more, 200 g ai/ha or more, 205 g ai/ha or more, 210 g ai/ha or more, 215 g ai/ha or more, 220 g ai/ha or more, 225 g ai/ha or more, 230 g ai/ha or more, or 235 g ai/ha or more; in an amount of 240 g ai/ha or less, such as 235 g ai/ha or less, 230 g ai/ha or less, 225 g ai/ha or less, 220 g ai/ha or less, 215 g ai/ha or less, 210 g ai/ha or less, 205 g ai/ha or less, 200 g ai/ha or less, 195 g ai/ha or less, 190 g ai/ha or less, 185 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 145 g ai/ha or less, 140 g ai/ha or less, 135 g ai/ha or less, 130 g ai/ha or less, 125 g ai/ha or less, 120 g ai/ha or less, 115 g ai/ha or less, 110 g ai/ha or less, 105 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, or 45 g ai/ha or less; or in an amount ranging from any of the minimum values described above to any of the maximum values described above, such as 40-240 g ai/ha, 40-230 g ai/ha, 40-220 g ai/ha, 40-210 g ai/ha, 40-200 g ai/ha, 50-240 g ai/ha, 50-230 g ai/ha, 50-220 g ai/ha, 50-210 g ai/ha, 50-200 g ai/ha, 60-240 g ai/ha, 60-230 g ai/ha, 60-220 g ai/ha, 60-210 g ai/ha, or 60-200 g ai/ha.

### Thenylchlor

In some aspects, the VLCFA synthesis inhibitor herbicide can comprise thenylchlor or an agriculturally acceptable salt or ester thereof. Thenylchlor, shown below, is 2-chloro-N-(2,6-dimethylphenyl)-N-[(3-methoxy-2-thienyl)methyl]acetamide. Its herbicidal activity is exemplified in The Pesticide Manual, Seventeenth Edition, 2016. Exemplary uses of thenylchlor include its use for pre-emergence control of annual grass and broadleaf weeds in paddy rice.

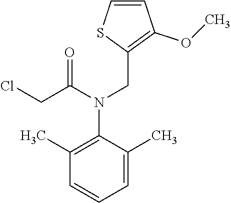 [SMILES: COc1ccsc1CN(C(=O)CCl)c1c(C)cccc1C]

Thenylchlor can be applied to vegetation or an area adjacent the vegetation or applied to soil or water to prevent the emergence or growth of vegetation in an amount sufficient to induce a herbicidal effect. In some aspects, thenylchlor is applied to vegetation or an area adjacent the vegetation or applied to soil or water to prevent the emergence or growth of vegetation in an amount of 225 g ai/ha or more, such as 230 g ai/ha or more, 235 g ai/ha or more, 240 g ai/ha or more, 245 g ai/ha or more, 250 g ai/ha or more, 255 g ai/ha or more, 260 g ai/ha or more, 265 g ai/ha or more, 270 g ai/ha or more, 275 g ai/ha or more, 280 g ai/ha or more, 285 g ai/ha or more, 290 g ai/ha or more, 295 g ai/ha or more, 300 g ai/ha or more, 305 g ai/ha or more, 310 g ai/ha or more, 315 g ai/ha or more, or 320 g ai/ha or more; in an amount of 325 g ai/ha or less, such as 320 g ai/ha or less, 315 g ai/ha or less, 310 g ai/ha or less, 305 g ai/ha or less, 300 g ai/ha or less, 295 g ai/ha or less, 290 g ai/ha or less, 285 g ai/ha or less, 280 g ai/ha or less, 275 g ai/ha or less, 270 g ai/ha or less, 265 g ai/ha or less, 260 g ai/ha or less, 255 g ai/ha or less, 250 g ai/ha or less, 245 g ai/ha or less, 240 g ai/ha or less, 235 g ai/ha or less, or 230 g ai/ha or less; or in an amount ranging from any of the minimum values described above to any of the maximum values described above, such as 225-325 g ai/ha, 240-295 g ai/ha, 265-310 g ai/ha, 300-320 g ai/ha, 270-315 g ai/ha, 250-305 g ai/ha, 235-300 g ai/ha, or 230-270 g ai/ha.

### Microtubule Assembly Inhibitor (MAI) Herbicides

In addition to the compounds according to Formula I or agriculturally acceptable N-oxide, salt or ester thereof, the compositions can include a microtubule assembly inhibitor (MAI) herbicide, an agriculturally acceptable salt or ester thereof, or mixtures thereof. MAI herbicides may inhibit plant cell division by binding to tubulin, the major protein needed to form the microtubules required in cell division.

Examples of MAI herbicides include benfluralin, butamifos, butralin, carbetamide, chlorpropham, chlorthal, dithiopyr, ethalfluralin, oryzalin, pendimethalin, prodiamine, propham, propyzamide, thiazopyr, trifluralin, and agriculturally acceptable salts or esters thereof.

MAI herbicides can be applied to vegetation or an area adjacent the vegetation or applied to soil or water to prevent the emergence or growth of vegetation in an amount sufficient to induce a herbicidal effect. In some aspects, the MAI herbicide is applied to vegetation or an area adjacent the vegetation or applied to soil or water to prevent the emergence or growth of vegetation in an amount of 100 g ai/ha or more, such as 105 g ai/ha or more, 110 g ai/ha or more, 115 g ai/ha or more, 120 g ai/ha or more, 125 g ai/ha or more, 130 g ai/ha or more, 135 g ai/ha or more, 140 g ai/ha or more, 145 g ai/ha or more, 150 g ai/ha or more, 155 g ai/ha or more, 160 g ai/ha or more, 165 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 500 g ai/ha or more, 600 g ai/ha or more, 700 g ai/ha or more, 750 g ai/ha or more, 800 g ai/ha or more, 900 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more, 3400 g ai/ha or more, 3500 g ai/ha or more, 3750 g ai/ha or more, 4000 g ai/ha or more, 4250 g ai/ha or more, 4500 g ai/ha or more, 4750 g ai/ha or more, 5000 g ai/ha or more, 5250 g ai/ha or more, 5500 g ai/ha or more, 5750 g ai/ha or more, 6000 g ai/ha or more, 6250 g ai/ha or more, 6500 g ai/ha or more, 6750 g ai/ha or more, 7000 g ai/ha or more, 7250 g ai/ha or more, 7500 g ai/ha or more, 7750 g ai/ha or more, 8000 g ai/ha or more, 8250 g ai/ha or more, 8500 g ai/ha or more, 8750 g ai/ha or more, 9000 g ai/ha or more, 9250 g ai/ha or more, 9500 g ai/ha or more, 9750 g ai/ha or more, 10 kg ai/ha or more, 10.5 kg ai/ha or more, 11 kg ai/ha or more, 11.5 kg ai/ha or more, 12 kg ai/ha or more, 12.5 kg ai/ha or more, 13 kg ai/ha or more, 13.5 kg ai/ha or more, 14 kg ai/ha or more, 14.5 kg ai/ha or more, 15 kg ai/ha or more, 15.5 kg ai/ha or more, 16 kg ai/ha or more, 16.5 kg ai/ha or more, or 16.75 kg ai/ha or more; in an amount of 17 kg ai/ha or less, such as 16.75 kg ai/ha or less, 16.5 kg ai/ha or less, 16 kg ai/ha or less, 15.5 kg ai/ha or less, 15 kg ai/ha or less, 14.5 kg ai/ha or less, 14 kg ai/ha or less, 13.5 kg ai/ha or less, 13 kg ai/ha or less, 12.5 kg ai/ha or less, 12 kg ai/ha or less, 11.5 kg ai/ha or less, 11 kg ai/ha or less, 10.5 kg ai/ha, 10 kg ai/ha or less, 9750 g ai/ha or less, 9500 g ai/ha or less, 9250 g ai/ha or less, 9000 g ai/ha or less, 8750 g ai/ha or less, 8500 g ai/ha or less, 8250 g ai/ha or less, 8000 g ai/ha or less, 7750 g ai/ha or less, 7500 g ai/ha or less, 7250 g ai/ha or less, 7000 g ai/ha or less, 6750 g ai/ha or less, 6500 g ai/ha or less, 6250 g ai/ha or less, 6000 g ai/ha or less, 5750 g ai/ha or less, 5500 g ai/ha or less, 5250 g ai/ha or less, 5000 g ai/ha or less, 4750 g ai/ha or less, 4500 g ai/ha or less, 4250 g ai/ha or less, 4000 g ai/ha or less, 3750 g ai/ha or less, 3600 g ai/ha or less, 3500 g ai/ha or less, 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 900 g ai/ha or less, 800 g ai/ha or less, 750 g ai/ha or less, 700 g ai/ha or less, 600 g ai/ha or less, 500 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 165 g ai/ha or less, 160 g ai/ha or less, 155 g ai/ha or less, 150 g ai/ha or less, 145 g ai/ha or less, 140 g ai/ha or less, 135 g ai/ha or less, 130 g ai/ha or less, 125 g ai/ha or less, 120 g ai/ha or less, 115 g ai/ha or less, 110 g ai/ha or less, or 105 g ai/ha or less; or in an amount ranging from any of the minimum values described above to any of the maximum values described above, e.g., 100 g-17 kg ai/ha, 350-1800 g ai/ha, 160-3750 g ai/ha, 225-2500 g ai/ha, 7.5-12.5 kg ai/ha, 115-350 g ai/ha, 3400-6750 g ai/ha, 185-8000 g ai/ha, 390-3100 g ai/ha, 2000-4250 g ai/ha, 1200-3300 g ai/ha, or 105 g-16.75 kg ai/ha.

### Benfluralin

In some aspects, the MAI herbicide can comprise benfluralin or an agriculturally acceptable salt or ester thereof. Benfluralin, shown below, is a dinitroaniline herbicide used for pre-plant incorporated control of annual grass and some annual broad-leaved weeds in peanuts, lettuce, cucumbers, chicory, endive, field beans, French beans, lentils, alfalfa, clovers, trefoil, tobacco and established turf, at 1000-1500 g/ha. Benfluralin, as well as methods of preparing benfluralin, are known in the art. Its herbicidal activity is described, for example, in Tomlin, C. D. S., Ed. The Pesticide Manual: A World Compendium, 17th ed.; BCPC: Alton, 2016 (hereafter “The Pesticide Manual, Seventeenth Edition, 2016”).

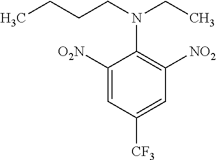 [SMILES: CCCCN(CC)c1c([N+](=O)[O-])cc(C(F)(F)F)cc1[N+](=O)[O-]]

Benfluralin can be applied to vegetation or an area adjacent the vegetation or applied to soil or water to prevent the emergence or growth of vegetation in an amount sufficient to induce a herbicidal effect. In some aspects, benfluralin is applied to vegetation or an area adjacent the vegetation or applied to soil or water to prevent the emergence or growth of vegetation in an amount of 900 g ai/ha or more, such as 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1440 g ai/ha or more, 1450 g ai/ha or more, 1500 g ai/ha or more, 1600 g ai/ha or more, or 1700 g ai/ha or more; in an amount of 1750 g ai/ha or less, such as 1700 g ai/ha or less, 1600 g ai/ha or less, 1500 g ai/ha or less, 1450 g ai/ha or less, 1440 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, or 950 g ai/ha or less; or in or in an amount ranging from any of the minimum values described above to any of the maximum values described above, such as 900-1750 g ai/ha, 1100-1350 g ai/ha, 1000-1500 g ai/ha, 1350-1600 g ai/ha, 1150-1400 g ai/ha, 950-1600 g ai/ha, 900-1440 g ai/ha, 1500-1700 g ai/ha, or 950-1700 g ai/ha.

### Butamifos

In some aspects, the MAI herbicide can comprise butamifos or an agriculturally acceptable salt or ester thereof. Butamifos, shown below, is a phosphoroamidate herbicide used for pre-emergence control of annual grass weeds in beans, turf, rice, at 900-1000 g/ha, and in vegetables, at 1000-2000 g/ha. Butamifos, as well as methods of preparing butamifos, are known in the art. Its herbicidal activity is described, for example, in The Pesticide Manual, Seventeenth Edition, 2016.

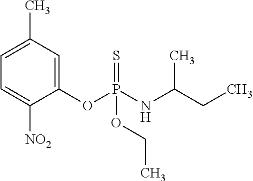 [SMILES: CCOP(=S)(NC(C)CC)Oc1cc(C)ccc1[N+](=O)[O-]]

Butamifos can be applied to vegetation or an area adjacent the vegetation or applied to soil or water to prevent the emergence or growth of vegetation in an amount sufficient to induce a herbicidal effect. In some aspects, butamifos is applied to vegetation or an area adjacent the vegetation or applied to soil or water to prevent the emergence or growth of vegetation in an amount of 1000 g ai/ha or more, such as 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, or 1950 g ai/ha or more; in an amount of 2000 g ai/ha or less, such as 1950 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, or 1050 g ai/ha or less; or in an amount ranging from any of the minimum values described above to any of the maximum values described above, such as 1000-2000 g ai/ha, 1400-1700 g ai/ha, 1600-1950 g ai/ha, 1150-1500 g ai/ha, 1250-1400 g ai/ha, 1900-2000 g ai/ha, 1050-1200 g ai/ha, 1300-1800 g ai/ha, 1200-1500 g ai/ha, or 1050-1950 g ai/ha.

### Butralin

In some aspects, the MAI herbicide can comprise butralin or an agriculturally acceptable salt or ester thereof. Butralin, shown below, is a dinitroaniline herbicide used for pre-emergence control of annual broad-leaved and grass weeds in cotton, soybeans, rice, barley, beans, alliums, vines, ornamentals, fruit and nut trees, at 1120-3400 g/ha; control of suckers in tobacco, at 125 mg/plant. Butralin, as well as methods of preparing butralin, are known in the art. Its herbicidal activity is described, for example, in The Pesticide Manual, Seventeenth

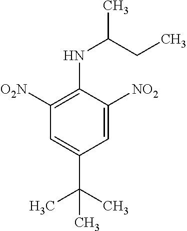 [SMILES: CCC(C)Nc1c([N+](=O)[O-])cc(C(C)(C)C)cc1[N+](=O)[O-]]

### Edition, 2016.

Butralin can be applied to vegetation or an area adjacent the vegetation or applied to soil or water to prevent the emergence or growth of vegetation in an amount sufficient to induce a herbicidal effect. In some aspects, butralin is applied to vegetation or an area adjacent the vegetation or applied to soil or water to prevent the emergence or growth of vegetation in an amount of 1100 g ai/ha or more, such as 1120 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3300 g ai/ha or more or 3400 g ai/ha or more; in an amount of 3500 g ai/ha or less, such as 3400 g ai/ha or less, 3300 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, or 1120 g ai/ha or less; or in an amount ranging from any of the minimum values described above to any of the maximum values described above, such as 1100-3500 g ai/ha, 1450-2900 g ai/ha, 1300-3200 g ai/ha, 1600-2500 g ai/ha, 1250-3000 g ai/ha, 1800-2400 g ai/ha, 1900-3400 g ai/ha, 1350-2700 g ai/ha, 1120-3100 g ai/ha, 1500-3500 g ai/ha, 2200-2900 g ai/ha, or 1120-3400 g ai/ha.

### Carbetamide

In some aspects, the MAI herbicide can comprise carbetamide or an agriculturally acceptable salt or ester thereof. Carbetamide, shown below, is a carbamate herbicide used for control of annual grass weeds, including volunteer cereals, and some broad-leaved weeds in clover, alfalfa, sainfoin, brassicas, field beans, peas, lentils, sugar beets, oilseed rape, chicory, endive, sunflowers, caraway, strawberries, vines and fruit, at 2000 g/ha. Carbetamide, as well as methods of preparing carbetamide, are known in the art. Its herbicidal activity is described, for example, in The Pesticide Manual, Seventeenth Edition, 2016.

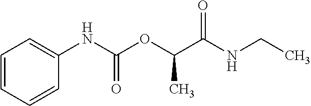 [SMILES: CCNC(=O)[C@@H](C)OC(=O)Nc1ccccc1]

Carbetamide can be applied to vegetation or an area adjacent the vegetation or applied to soil or water to prevent the emergence or growth of vegetation in an amount sufficient to induce a herbicidal effect. In some aspects, carbetamide is applied to vegetation or an area adjacent the vegetation or applied to soil or water to prevent the emergence or growth of vegetation in an amount of 900 g ai/ha or more, such as 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, or 1750 g ai/ha or more; in an amount of 1800 g ai/ha or less, such as 1750 g ai/ha or less, 1700 g ai/ha or less, 1600 g ai/ha or less, 1500 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, or 950 g ai/ha or less; or in an amount ranging from any of the minimum values described above to any of the maximum values described above, such as 900-1800 g ai/ha, 1400-1700 g ai/ha, 1500-1750 g ai/ha, 1150-1500 g ai/ha, 1250-1400 g ai/ha, 1000-1600 g ai/ha, 1050-1200 g ai/ha, 1300-1800 g ai/ha, 1200-1600 g ai/ha, or 950-1750 g ai/ha.

### Chlorpropham

In some aspects, the MAI herbicide can comprise chlorpropham or an agriculturally acceptable salt or ester thereof. Chlorpropham, shown below, is a carbamate herbicide used to inhibit sprouting in stored potatoes, at 8-64 g/t and control suckers in tobacco. Chlorpropham is also used for pre-emergence control of annual grass and some broad-leaved weeds in onions, leeks, garlic, shallots, alfalfa, clover, sugar beets, spinach, lettuce, endive, chicory, herbs, peas, beans, carrots, celery, black salsify, fennel, soybeans, cotton, rice, safflowers, sunflowers, cranberries, cane berries, ornamentals, ornamental trees and shrubs, perennial grass seed crops, etc., at up to 240 g/ha. Chlorpropham, as well as methods of preparing chlorpropham, are known in the art. Its herbicide activity is described, for example, in The Pesticide Manual, Seventeenth Edition, 2016.

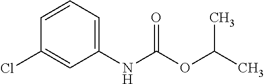

Chlorpropham can be applied to vegetation or an area adjacent the vegetation or applied to soil or water to prevent the emergence or growth of vegetation in an amount sufficient to induce a herbicidal effect. In some aspects, chlorpropham is applied to vegetation or an area adjacent the vegetation or applied to soil or water to prevent the emergence or growth of vegetation in an amount of 15 g ai/ha or more, such as 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, or 245 g ai/ha or more; in an amount of 250 g ai/ha or less, such as 245 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, or 20 g ai/ha or less; or in an amount ranging from any of the minimum values described above to any of the maximum values described above, such as 15-250 g ai/ha, 30-65 g ai/ha, 40-175 g ai/ha, 65-250 g ai/ha, 50-150 g ai/ha, 110-190 g ai/ha, 120-230 g ai/ha, 75-220 g ai/ha, 80-230 g ai/ha, 100-140 g ai/ha, 95-160 g ai/ha, or 20-245 g ai/ha.

### Chlorthal

In some aspects, the MAI herbicide can comprise chlorthal or an agriculturally acceptable salt or ester thereof. Chlorthal, shown below, is a benzoic acid herbicide used for preemergence control of annual grass and some broad-leaved weeds in onions, garlic, leeks, tomatoes, lettuce, cucurbits, peppers, aubergines, brassicas, potatoes, sweet potatoes, horseradish, field beans, soybeans, cotton, strawberries, ornamentals, established turf and other crops. One common ester of chlorthal is chlorthal-dimethyl. Chlorthal, as well as methods of preparing chlorthal, are known in the art. Its herbicide activity is described, for example, in The Pesticide Manual, Seventeenth Edition, 2016.

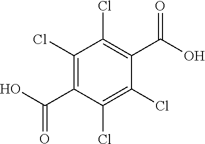 [SMILES: O=C(O)c1c(Cl)c(Cl)c(C(=O)O)c(Cl)c1Cl]

Chlorthal can be applied to vegetation or an area adjacent the vegetation or applied to soil or water to prevent the emergence or growth of vegetation in an amount sufficient to induce a herbicidal effect. In some aspects, chlorthal is applied to vegetation or an area adjacent the vegetation or applied to soil or water to prevent the emergence or growth of vegetation in an amount of 5000 g ai/ha or more, such as 5250 g ai/ha or more, 5500 g ai/ha or more, 5750 g ai/ha or more, 6000 g ai/ha or more, 6250 g ai/ha or more, 6500 g ai/ha or more, 6750 g ai/ha or more, 7000 g ai/ha or more, 7250 g ai/ha or more, 7500 g ai/ha or more, 7750 g ai/ha or more, 8000 g ai/ha or more, 8250 g ai/ha or more, 8500 g ai/ha or more, 8750 g ai/ha or more, 9000 g ai/ha or more, 9250 g ai/ha or more, 9500 g ai/ha or more, 9750 g ai/ha or more, 10 kg ai/ha or more, 10.5 kg ai/ha or more, 11 kg ai/ha or more, 11.5 kg ai/ha or more, 12 kg ai/ha or more, 12.5 kg ai/ha or more, 13 kg ai/ha or more, 13.5 kg ai/ha or more, 14 kg ai/ha or more, 14.5 kg ai/ha or more, 15 kg ai/ha or more, 15.5 kg ai/ha or more, 16 kg ai/ha or more, 16.5 kg ai/ha or more, or 16.75 kg ai/ha or more; in an amount of 17 kg ai/ha or less, such as 16.5 kg ai/ha or less, 16 kg ai/ha or less, 15.5 kg ai/ha or less, 15 kg ai/ha or less, 14.5 kg ai/ha or less, 14 kg ai/ha or less, 13.5 kg ai/ha or less, 13 kg ai/ha or less, 12.5 kg ai/ha or less, 12 kg ai/ha or less, 11.5 kg ai/ha or less, 11 kg ai/ha or less, 10.5 kg ai/ha, 10 kg ai/ha or less, 9750 g ai/ha or less, 9500 g ai/ha or less, 9250 g ai/ha or less, 9000 g ai/ha or less, 8750 g ai/ha or less, 8500 g ai/ha or less, 8250 g ai/ha or less, 8000 g ai/ha or less, 7750 g ai/ha or less, 7500 g ai/ha or less, 7250 g ai/ha or less, 7000 g ai/ha or less, 6750 g ai/ha or less, 6500 g ai/ha or less, 6250 g ai/ha or less, 6000 g ai/ha or less, 5750 g ai/ha or less, 5500 g ai/ha or less, or 5250 g ai/ha or less; or in an amount ranging from any of the minimum values described above to any of the maximum values described above, such as 5000 g-17.5 kg ai/ha, 6250-9500 g ai/ha, 5500-8750 g ai/ha, 6000 g-12.5 kg ai/ha, 10.5-15 kg ai/ha, 9250 g-14.5 kg ai/ha, 8000-10,000 g ai/ha, 7750 g-16 kg ai/ha, 500-1400 g ai/ha, 6500-8500 g ai/ha, or 5250 g-16.5 kg ai/ha.

### Dithiopyr

In some aspects, the MAI herbicide can comprise dithiopyr or an agriculturally acceptable salt or ester thereof. Dithiopyr, shown below, is a pyridine herbicide used for pre- and early post-emergence control of annual grass, e.g. crabgrass, and broad-leaved weeds in turf, at 280-1100 g/ha. Dithiopyr, as well as methods of preparing dithiopyr, are known in the art. Its herbicide activity is described, for example, in The Pesticide Manual, Seventeenth Edition, 2016.

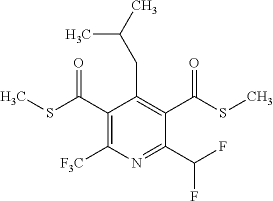

Dithiopyr can be applied to vegetation or an area adjacent the vegetation or applied to soil or water to prevent the emergence or growth of vegetation in an amount sufficient to induce a herbicidal effect. In some aspects, dithiopyr is applied to vegetation or an area adjacent the vegetation or applied to soil or water to prevent the emergence or growth of vegetation in an amount of 200 g ai/ha or more, such as 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, or 1200 g ai/ha or more; in an amount of 1250 g ai/ha or less, such as 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, or 210 g ai/ha or less; or in an amount ranging from any of the minimum values described above to any of the maximum values described above, such as 200-1250 g ai/ha, 350-650 g ai/ha, 460-750 g ai/ha, 600-1150 g ai/ha, 280-550 g ai/ha, 310-450 g ai/ha, 400-900 g ai/ha, 750-1200 g ai/ha, 430-850 g ai/ha, 500-1000 g ai/ha, 260-1100 g ai/ha, or 210-1200 g ai/ha.

### Ethalfluralin

In some aspects, the MAI herbicide can comprise ethalfluralin or an agriculturally acceptable salt or ester thereof. Ethalfluralin, shown below, is a dinitroaniline herbicide used for pre-plant incorporated control of annual grass and broad-leaved weeds in cotton, soybeans, dry beans, lentils, maize, sorghum, safflowers and sunflowers, at 1000-1250 g/ha; and as post-plant surface application for control of annual grass and broad-leaved weeds in peanuts and cucurbits. Ethalfluralin, as well as methods of preparing ethalfluralin, are known in the art. Its herbicide activity is described, for example, in The Pesticide Manual, Seventeenth Edition, 2016.

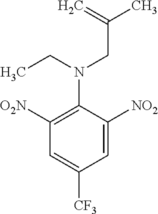 [SMILES: C=C(C)CN(CC)c1c([N+](=O)[O-])cc(C(F)(F)F)cc1[N+](=O)[O-]]

Ethalfluralin can be applied to vegetation or an area adjacent the vegetation or applied to soil or water to prevent the emergence or growth of vegetation in an amount sufficient to induce a herbicidal effect. In some aspects, ethalfluralin is applied to vegetation or an area adjacent the vegetation or applied to soil or water to prevent the emergence or growth of vegetation in an amount of 750 g ai/ha or more, such as 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, or 1450 g ai/ha or more; in an amount of 1500 g ai/ha or less, such as 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, or 800 g ai/ha or less; or in an amount ranging from any of the minimum values described above to any of the maximum values described above, such as 750-1500 g ai/ha, 950-1200 g ai/ha, 850-1250 g ai/ha, 1050-1300 g ai/ha, 800-1400 g ai/ha, 1100-1450 g ai/ha, 900-1150 g ai/ha, 750-1200 g ai/ha, 1350-1450 g ai/ha, 1000-1400 g ai/ha, or 800-1450 g ai/ha.

### Oryzalin

In some aspects, the MAI herbicide can comprise oryzalin or an agriculturally acceptable salt or ester thereof. Oryzalin, shown below, is a dinitroaniline herbicide used for pre-emergence control of annual grass and broad-leaved weeds in cotton, at 720-960 g/ha, soybeans, at 960-2160 g/ha, rice, at 240-480 g/ha, tree fruit, nut trees, vines, ornamentals, berries, amenity turf and non-crop areas, at 1920-4500 g/ha. Oryzalin, as well as methods of preparing oryzalin, are known in the art. Its herbicide activity is described, for example, in The Pesticide Manual, Seventeenth Edition, 2016.

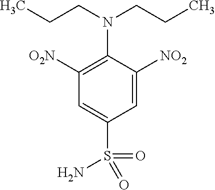 [SMILES: CCCN(CCC)c1c([N+](=O)[O-])cc(S(N)(=O)=O)cc1[N+](=O)[O-]]

Oryzalin can be applied to vegetation or an area adjacent the vegetation or applied to soil or water to prevent the emergence or growth of vegetation in an amount sufficient to induce a herbicidal effect. In some aspects, oryzalin is applied to vegetation or an area adjacent the vegetation or applied to soil or water to prevent the emergence or growth of vegetation in an amount of 200 g ai/ha or more, such as 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, or 1450 g ai/ha or more; in an amount of 1500 g ai/ha or less, such as 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, or 210 g ai/ha or less; or in an amount ranging from any of the minimum values described above to any of the maximum values described above, such as 200-1500 g ai/ha, 350-650 g ai/ha, 460-750 g ai/ha, 600-1150 g ai/ha, 280-550 g ai/ha, 310-450 g ai/ha, 400-900 g ai/ha, 750-1300 g ai/ha, 430-850 g ai/ha, 500-1450 g ai/ha, 260-1200 g ai/ha, or 210-1450 g ai/ha.

### Pendimethalin

In some aspects, the MAI herbicide can comprise pendimethalin or an agriculturally acceptable salt or ester thereof. Pendimethalin, shown below, is a dinitroaniline herbicide used for pre-plant incorporated, pre- and early post-emergence control of annual grass and broad-leaved weeds in cereals, onions, leeks, garlic, fennel, maize, sorghum, rice, soybeans, peanuts, brassicas, carrots, celery, black salsify, peas, field beans, lupins, evening primroses, tulips, potatoes, cotton, hops, pome fruit, stone fruit, berries, strawberries, citrus, lettuce, aubergines, peppers, established turf and in transplanted tomatoes, sunflowers and tobacco, at 600-2400 g/ha; control of suckers in tobacco. Pendimethalin, as well as methods of preparing pendimethalin, are known in the art. Its herbicide activity is described, for example, in The Pesticide Manual, Seventeenth Edition, 2016.

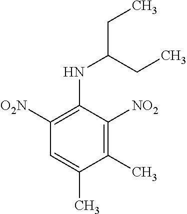 [SMILES: CCC(CC)Nc1c([N+](=O)[O-])cc(C)c(C)c1[N+](=O)[O-]]

Pendimethalin can be applied to vegetation or an area adjacent the vegetation or applied to soil or water to prevent the emergence or growth of vegetation in an amount sufficient to induce a herbicidal effect. In some aspects, pendimethalin is applied to vegetation or an area adjacent the vegetation or applied to soil or water to prevent the emergence or growth of vegetation in an amount of 500 g ai/ha or more, such as 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1750 g ai/ha or more, 1800 g ai/ha or more, 1900 g ai/ha or more, 2000 g ai/ha or more, 2100 g ai/ha or more, 2200 g ai/ha or more, 2300 g ai/ha or more, or 2400 g ai/ha or more; in an amount of 2500 g ai/ha or less, such as 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, or 525 g ai/ha or less; or in an amount ranging from any of the minimum values described above to any of the maximum values described above, such as 500-2500 g ai/ha, 950-1800 g ai/ha, 625-1750 g ai/ha, 900-1900 g ai/ha, 850-2300 g ai/ha, 1150-1450 g ai/ha, 1900-2000 g ai/ha, 750-2200 g ai/ha, 1350-2100 g ai/ha, 575-1400 g ai/ha, 675-900 g ai/ha, or 525-2400 g ai/ha

### Prodiamine

In some aspects, the MAI herbicide can comprise prodiamine or an agriculturally acceptable salt or ester thereof. Prodiamine, shown below, is a dinitroaniline herbicide used for pre-plant or pre-emergence control of annual grass and broad-leaved weeds in non-crop land, ornamentals, turf, conifer and hardwood seedlings, at 375-1600 g/ha. Prodiamine, as well as methods of preparing prodiamine, are known in the art. Its herbicide activity is described, for example, in The Pesticide Manual, Seventeenth Edition, 2016.

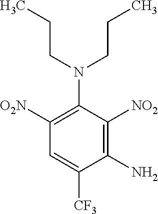 [SMILES: CCCN(CCC)c1c([N+](=O)[O-])cc(C(F)(F)F)c(N)c1[N+](=O)[O-]]

Prodiamine can be applied to vegetation or an area adjacent the vegetation or applied to soil or water to prevent the emergence or growth of vegetation in an amount sufficient to induce a herbicidal effect. In some aspects, prodiamine is applied to vegetation or an area adjacent the vegetation or applied to soil or water to prevent the emergence or growth of vegetation in an amount of 350 g ai/ha or more, such as 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, or 1750 g ai/ha or more; in an amount of 1800 g ai/ha or less, such as 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, or 360 g ai/ha or less; or in an amount ranging from any of the minimum values described above to any of the maximum values described above, such as 350-1800 g ai/ha, 430-650 g ai/ha, 550-1350 g ai/ha, 600-1500 g ai/ha, 375-1300 g ai/ha, 675-1150 g ai/ha, 900-1450 g ai/ha, 750-1200 g ai/ha, 380-1700 g ai/ha, 500-1400 g ai/ha, 440-850 g ai/ha, or 360-1750 g ai/ha.

### Propham

In some aspects, the MAI herbicide can comprise propham or an agriculturally acceptable salt or ester thereof. Propham, shown below, is a carbamate herbicide used for control of annual grass and some broad-leaved weeds in alfalfa, clover, sugar beets, spinach, lettuce, peas, beans, flax, safflowers, beetroot, fodder beets, lettuce, chard, lentils and perennial grass seed crops, at 2300-5000 g/ha, and as a sprouting inhibitor in potatoes. Propham, as well as methods of preparing propham, are known in the art. Its herbicide activity is described, for example, in The Pesticide Manual, Eighteenth Edition, 2016.

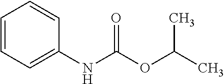

Propham can be applied to vegetation or an area adjacent the vegetation or applied to soil or water to prevent the emergence or growth of vegetation in an amount sufficient to induce a herbicidal effect. In some aspects, propham is applied to vegetation or an area adjacent the vegetation or applied to soil or water to prevent the emergence or growth of vegetation in an amount of 2000 g ai/ha or more, such as 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3750 g ai/ha or more, 4000 g ai/ha or more, 4250 g ai/ha or more, 4500 g ai/ha or more, 4750 g ai/ha or more, 5000 g ai/ha or more, 5250 g ai/ha or more, 5500 g ai/ha or more, 5750 g ai/ha or more, 6000 g ai/ha or more, 6250 g ai/ha or more, 6500 g ai/ha or more, or 6750 g ai/ha or more; in an amount of 7000 g ai/ha or less, such as 6750 g ai/ha or less, 6500 g ai/ha or less, 6250 g ai/ha or less, 6000 g ai/ha or less, 5750 g ai/ha or less, 5500 g ai/ha or less, 5250 g ai/ha or less, 5000 g ai/ha or less, 4750 g ai/ha or less, 4500 g ai/ha or less, 4250 g ai/ha or less, 4000 g ai/ha or less, 3750 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, or 2100 g ai/ha or less; or in an amount ranging from any of the minimum values described above to any of the maximum values described above, such as 2000-7000 g ai/ha, 4750-6500 g ai/ha, 2300-3750 g ai/ha, 4000-6000 g ai/ha, 2700-5000 g ai/ha, 3100-4750 g ai/ha, 2500-4000 g ai/ha, 5750-7000 g ai/ha, 3300-5250 g ai/ha, 2000-4500 g ai/ha, 2900-5500 g ai/ha, or 2100-6750 g ai/ha.

### Propyzamide

In some aspects, the MAI herbicide can comprise propyzamide or an agriculturally acceptable salt or ester thereof. Propyzamide, shown below, is a benzamide herbicide used for pre- and early post-emergence control of annual and perennial grass and some broad-leaved weeds, in fruit, vines, lettuce, endive, chicory, brassicas, oilseed rape, legumes, alfalfa, clover, trefoil, sainfoin, artichokes, sugar beets, roses, ornamental trees and shrubs, warm season amenity grass, fallow land and forestry and Poa annua in Bermuda grass, zoysia grass and certain other turf species, at 560-2200 g/ha. Propyzamide, as well as methods of preparing propyzamide, are known in the art. Its herbicide activity is described, for example, in The Pesticide Manual, Seventeenth Edition, 2016.

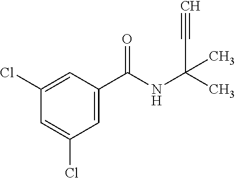 [SMILES: C#CC(C)(C)NC(=O)c1cc(Cl)cc(Cl)c1]

Propyzamide can be applied to vegetation or an area adjacent the vegetation or applied to soil or water to prevent the emergence or growth of vegetation in an amount sufficient to induce a herbicidal effect. In some aspects, propyzamide is applied to vegetation or an area adjacent the vegetation or applied to soil or water to prevent the emergence or growth of vegetation in an amount of 250 g ai/ha or more, such as 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1750 g ai/ha or more, 1800 g ai/ha or more, 1900 g ai/ha or more, 2000 g ai/ha or more, 2100 g ai/ha or more, 2200 g ai/ha or more, 2300 g ai/ha or more, or 2400 g ai/ha or more; in an amount of 2500 g ai/ha or less, such as 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g

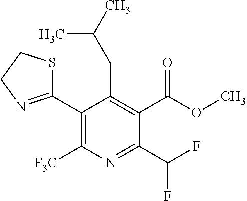 [SMILES: COC(=O)c1c(C(F)F)nc(C(F)(F)F)c(C2=NCCS2)c1CC(C)C]

ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, or 260 g ai/ha or less; or in an amount ranging from any of the minimum values described above to any of the maximum values described above, such as 250-2500 g ai/ha, 950-1900 g ai/ha, 360-1750 g ai/ha, 600-2000 g ai/ha, 550-1350 g ai/ha, 1100-1450 g ai/ha, 1900-2400 g ai/ha, 270-2200 g ai/ha, 380-1050 g ai/ha, 700-1700 g ai/ha, 440-1600 g ai/ha, or 260-2400 g ai/ha.

### Thiazopyr

In some aspects, the MAI herbicide can comprise thiazopyr or an agriculturally acceptable salt or ester thereof. Thiazopyr, shown below, is a pyridine herbicide used for pre-emergence control of annual grass and some broad-leaved weeds in tree fruit, vines, citrus, sugar cane, pineapples, alfalfa and forestry, at 100-560 g/ha. Thiazopyr, as well as methods of preparing thiazopyr, are known in the art. Its herbicide activity is described, for example, in The Pesticide Manual, Seventeenth Edition, 2016.

Thiazopyr can be applied to vegetation or an area adjacent the vegetation or applied to soil or water to prevent the emergence or growth of vegetation in an amount sufficient to induce a herbicidal effect. In some aspects, thiazopyr is applied to vegetation or an area adjacent the vegetation or applied to soil or water to prevent the emergence or growth of vegetation in an amount of 100 g ai/ha or more, such as 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, or 575 g ai/ha or more; in an amount of 600 g ai/ha or less, such as 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, or 110 g ai/ha or less; or in an amount ranging from any of the minimum values described above to any of the maximum values described above, such as 100-600 g ai/ha, 210-470 g ai/ha, 360-550 g ai/ha, 200-430 g ai/ha, 280-575 g ai/ha, 310-450 g ai/ha, 400-600 g ai/ha, 125-370 g ai/ha, 430-525 g ai/ha, 300-470 g ai/ha, 160-500 g ai/ha, or 110-575 g ai/ha.

### Trifluralin

In some aspects, the MAI herbicide can comprise trifluralin or an agriculturally acceptable salt or ester thereof. Trifluralin, shown below, is a dinitroaniline herbicide used for pre-plant incorporated or pre-emergence control of annual grass and broad-leaved weeds in brassicas, beans, peas, carrots, parsnips, lettuce, peppers, tomatoes, artichokes, onions, garlic, vines, strawberries, raspberries, citrus, oilseed rape, peanuts, soybeans, sunflowers, safflowers, ornamentals, including trees and shrubs, cotton, sugar beets, sugar cane, winter cereals, amenity grass and forestry, at 500-1000 g/ha. Trifluralin, as well as methods of preparing trifluralin, are known in the art. Its herbicide activity is described, for example, in The Pesticide Manual, Seventeenth Edition, 2016.

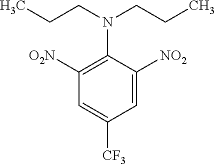 [SMILES: CCCN(CCC)c1c([N+](=O)[O-])cc(C(F)(F)F)cc1[N+](=O)[O-]]

Trifluralin can be applied to vegetation or an area adjacent the vegetation or applied to soil or water to prevent the emergence or growth of vegetation in an amount sufficient to induce a herbicidal effect. In some aspects, trifluralin is applied to vegetation or an area adjacent the vegetation or applied to soil or water to prevent the emergence or growth of vegetation in an amount of 400 g ai/ha or more, such as 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, or 1150 g ai/ha or more; in an amount of 1200 g ai/ha or less, such as 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, or 410 g ai/ha or less; or in an amount ranging from any of the minimum values described above to any of the maximum values described above, such as 400-1200 g ai/ha, 490-650 g ai/ha, 625-1150 g ai/ha, 500-1200 g ai/ha, 750-1000 g ai/ha, 410-850 g ai/ha, 800-1050 g ai/ha, 700-1100 g ai/ha, 440-900 g ai/ha, 400-1000 g ai/ha, 575-950 g ai/ha, or 410-1150 g ai/ha.

### Fatty Acid and Lipid Synthesis Inhibitor (FA/LSI) Herbicides

In addition to the compounds according to Formula I or agriculturally acceptable N-oxide, salt or ester thereof, the compositions can include a fatty acid and lipid synthesis inhibitor (FA/LSI) herbicide, an agriculturally acceptable salt or ester thereof, or mixtures thereof. FA/LSI herbicides appear to interfere with the biosynthesis of fatty acids and lipids, thereby reducing the deposition of cuticle wax, and to cause abnormal cell development or to inhibit cell division in germinating seedlings. Examples of FA/LSI herbicides include benfuresate, bensulide, butylate, cycloate, dalapon, EPTC, esprocarb, ethofumesate, flupropanate, molinate, orbencarb, prosulfocarb, thiobencarb, tiocarbazil, tri-allate, vernolate.

In some aspects, the composition can include a FA/LSI herbicide selected from the group consisting of benfuresate, bensulide, butylate, cycloate, dalapon, EPTC, esprocarb, ethofumesate, flupropanate, molinate, orbencarb, prosulfocarb, thiobencarb, tiocarbazil, tri-allate, vernolate, agriculturally acceptable salts or esters thereof, and mixtures thereof.

FA/LSI herbicides can be applied to vegetation or an area adjacent the vegetation or applied to soil or water to prevent the emergence or growth of vegetation in an amount sufficient to induce a herbicidal effect. In some aspects, the FA/LSI herbicide is applied to vegetation or an area adjacent the vegetation or applied to soil or water to prevent the emergence or growth of vegetation in an amount of 300 g ai/ha or more, such as 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1100 g ai/ha or more, 1200 g ai/ha or more, 1300 g ai/ha or more, 1400 g ai/ha or more, 1500 g ai/ha or more, 1600 g ai/ha or more, 1700 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3250 g ai/ha or more, 3500 g ai/ha or more, 3750 g ai/ha or more, 4000 g ai/ha or more, 4250 g ai/ha or more, 4500 g ai/ha or more, 4750 g ai/ha or more, 5000 g ai/ha or more, 5250 g ai/ha or more, 5500 g ai/ha or more, 5750 g ai/ha or more, 5000 g ai/ha or more, 5250 g ai/ha or more, 5500 g ai/ha or more, 5750 g ai/ha or more, 6000 g ai/ha or more, 6250 g ai/ha or more, 6500 g ai/ha or more, 6750 g ai/ha or more, 7000 g ai/ha or more, 7250 g ai/ha or more, 7500 g ai/ha or more, 7750 g ai/ha or more, 8000 g ai/ha or more, 8250 g ai/ha or more, 8500 g ai/ha or more, 8750 g ai/ha or more, 9000 g ai/ha or more, 9250 g ai/ha or more, 9500 g ai/ha or more, 9750 g ai/ha or more, 10 kg ai/ha or more, 10.5 kg ai/ha or more, 11 kg ai/ha or more, 11.5 kg ai/ha or more, 12 kg ai/ha or more, 12.5 kg ai/ha or more, 13 kg ai/ha or more, 13.5 kg ai/ha or more, 14 kg ai/ha or more, 14.5 kg ai/ha or more, 15 kg ai/ha or more, 15.5 kg ai/ha or more, 16 kg ai/ha or more, 16.5 kg ai/ha or more, 16.75 kg ai/ha or more, 17 kg ai/ha or more, 17.5 kg ai/ha or more, 18 kg ai/ha or more, 18.5 kg ai/ha or more, 19 kg ai/ha or more, 19.5 kg ai/ha or more, 20 kg ai/ha or more, or 20.5 kg ai/ha or more; in an amount of 21 kg ai/ha or less, such as 20.5 kg ai/ha or less, 20 kg ai/ha or less, 19.5 kg ai/ha or less, 19 kg ai/ha or less, 18.5 kg ai/ha or less, 18 kg ai/ha or less, 17.5 kg ai/ha or less, 17 kg ai/ha or less, 16.5 kg ai/ha or less, 16 kg ai/ha or less, 15.5 kg ai/ha or less, 15 kg ai/ha or less, 14.5 kg ai/ha or less, 14 kg ai/ha or less, 13.5 kg ai/ha or less, 13 kg ai/ha or less, 12.5 kg ai/ha or less, 12 kg ai/ha or less, 11.5 kg ai/ha or less, 11 kg ai/ha or less, 10.5 kg ai/ha, 10 kg ai/ha or less, 9750 g ai/ha or less, 9500 g ai/ha or less, 9250 g ai/ha or less, 9000 g ai/ha or less, 8750 g ai/ha or less, 8500 g ai/ha or less, 8250 g ai/ha or less, 8000 g ai/ha or less, 7750 g ai/ha or less, 7500 g ai/ha or less, 7250 g ai/ha or less, 7000 g ai/ha or less, 6750 g ai/ha or less, 6500 g ai/ha or less, 6250 g ai/ha or less, 6000 g ai/ha or less, 6750 g ai/ha or less, 6500 g ai/ha or less, 5250 g ai/ha or less, 5000 g ai/ha or less, 4750 g ai/ha or less, 4500 g ai/ha or less, 4250 g ai/ha or less, 4000 g ai/ha or less, 3750 g ai/ha or less, 3500 g ai/ha or less, 3250 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1700 g ai/ha or less, 1600 g ai/ha or less, 1500 g ai/ha or less, 1400 g ai/ha or less, 1300 g ai/ha or less, 1200 g ai/ha or less, 1100 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, or 310 g ai/ha or less; or in an amount ranging from any of the minimum values described above to any of the maximum values described above, such as 300 g-21 kg ai/ha, 950-4500 g ai/ha, 460-3750 g ai/ha, 6-15 kg ai/ha, 900-1700 g ai/ha, 320-450 g ai/ha, 8500 g-14 kg ai/ha, 750-2200 g ai/ha, 390-5250 g ai/ha, 400-1200 g ai/ha, 1200-3500 g ai/ha, or 310 g-20.5 kg ai/ha.

### Benfuresate

In some aspects, the FA/LSI herbicide can comprise benfuresate or an agriculturally acceptable salt or ester thereof. Benfuresate, shown below, is a benzofuran herbicide used for post-emergence control of grass and broad-leaved weeds in paddy rice, fruit, beans, maize, sugar cane and perennial crops, at 450-600 g/ha and pre-plant incorporated in cotton and tobacco, at 2000-3000 g/ha. Benfuresate, as well as methods of preparing benfuresate, are known in the art. Its herbicidal activity is described, for example, in Tomlin, C. D. S., Ed. The Pesticide Manual: A World Compendium, 17th ed.; BCPC: Alton, 2016 (hereafter “The Pesticide Manual, Seventeenth Edition, 2016”).

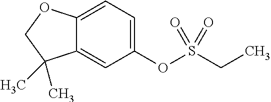 [SMILES: CCS(=O)(=O)Oc1ccc2c(c1)C(C)(C)CO2]

Benfuresate can be applied to vegetation or an area adjacent the vegetation or applied to soil or water to prevent the emergence or growth of vegetation in an amount sufficient to induce a herbicidal effect. In some aspects, benfuresate is applied to vegetation or an area adjacent the vegetation or applied to soil or water to prevent the emergence or growth of vegetation in an amount of 150 g ai/ha or more, such as 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1750 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, or 2900 g ai/ha or more; in an amount of 3000 g ai/ha or less, such as 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, or 160 g ai/ha or less; or in an amount ranging from any of the minimum values described above to any of the maximum values described above, such as 150-3000 g ai/ha, 950-2800 g ai/ha, 575-850 g ai/ha, 250-2500 g ai/ha, 1350-2700 g ai/ha, 1150-2300 g ai/ha, 1900-2600 g ai/ha, 270-1200 g ai/ha, 480-5250 g ai/ha, 500-1400 g ai/ha, 440-1700 g ai/ha, or 160-2900 g ai/ha.

### Bensulide

In some aspects, the FA/LSI herbicide can comprise bensulide or an agriculturally acceptable salt or ester thereof. Bensulide, shown below, is a phosphorodithioate herbicide used for pre-emergence control of broad-leaved weeds in brassicas, cucurbits, lettuce, onions, etc., at 5600-6700 g/ha, and in established turf, at 8400-28000 g/ha per season. Bensulide, as well as methods of preparing bensulide, are known in the art. Its herbicidal activity is described, for example, in The Pesticide Manual, Seventeenth Edition, 2016.

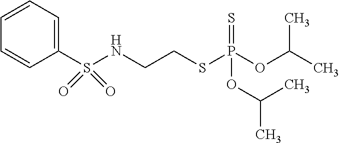 [SMILES: CC(C)OP(=S)(OC(C)C)SCCNS(=O)(=O)c1ccccc1]

Bensulide can be applied to vegetation or an area adjacent the vegetation or applied to soil or water to prevent the emergence or growth of vegetation in an amount sufficient to induce a herbicidal effect. In some aspects, bensulide is applied to vegetation or an area adjacent the vegetation or applied to soil or water to prevent the emergence or growth of vegetation in an amount of 5000 g ai/ha or more, such as 5250 g ai/ha or more, 5500 g ai/ha or more, 5750 g ai/ha or more, 6000 g ai/ha or more, 6250 g ai/ha or more, 6500 g ai/ha or more, 6750 g ai/ha or more, 7000 g ai/ha or more, 7250 g ai/ha or more, 7500 g ai/ha or more, 7750 g ai/ha or more, 8000 g ai/ha or more, 8250 g ai/ha or more, 8500 g ai/ha or more, 8750 g ai/ha or more, 9000 g ai/ha or more, 9250 g ai/ha or more, 9500 g ai/ha or more, 9750 g ai/ha or more, 10 kg ai/ha or more, 10.5 kg ai/ha or more, 11 kg ai/ha or more, 11.5 kg ai/ha or more, 12 kg ai/ha or more, 12.5 kg ai/ha or more, 13 kg ai/ha or more, or 13.5 kg ai/ha or more; in an amount of 14 kg ai/ha or less, such as 13.5 kg ai/ha or less, 13 kg ai/ha or less, 12.5 kg ai/ha or less, 12 kg ai/ha or less, 11.5 kg ai/ha or less, 11 kg ai/ha or less, 10.5 kg ai/ha, 10 kg ai/ha or less, 9750 g ai/ha or less, 9500 g ai/ha or less, 9250 g ai/ha or less, 9000 g ai/ha or less, 8750 g ai/ha or less, 8500 g ai/ha or less, 8250 g ai/ha or less, 8000 g ai/ha or less, 7750 g ai/ha or less, 7500 g ai/ha or less, 7250 g ai/ha or less, 7000 g ai/ha or less, 6750 g ai/ha or less, 6500 g ai/ha or less, 6250 g ai/ha or less, 6000 g ai/ha or less, 5750 g ai/ha or less, 5500 g ai/ha or less, or 5250 g ai/ha or less; or in an amount ranging from any of the minimum values described above to any of the maximum values described above, such as 5-15 kg ai/ha, 9500 g-13 kg ai/ha, 6250-8750 g ai/ha, 7-12 kg ai/ha, 8500-9750 g ai/ha, 11-13.5 kg ai/ha, 5750-7750 g ai/ha, 9750 g-12 kg ai/ha, 10-11 kg ai/ha, 7500-9500 g ai/ha, or 5250 g-13.5 kg ai/ha.

### Butylate

In some aspects, the FA/LSI herbicide can comprise butylate or an agriculturally acceptable salt or ester thereof. Butylate, shown below, is a thiocarbamate herbicide used for pre-plant incorporated control of annual grass weeds and Cyperus spp. in maize and pineapples at 3000-4000 g/ha. Butylate, as well as methods of preparing butylate, are known in the art. Its herbicidal activity is described, for example, in The Pesticide Manual, Seventeenth Edition, 2016.

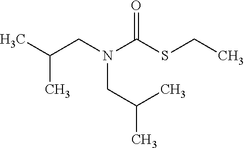

Butylate can be applied to vegetation or an area adjacent the vegetation or applied to soil or water to prevent the emergence or growth of vegetation in an amount sufficient to induce a herbicidal effect. In some aspects, butylate is applied to vegetation or an area adjacent the vegetation or applied to soil or water to prevent the emergence or growth of vegetation in an amount of 3000 g ai/ha or more, such as 3100 g ai/ha or more, 3200 g ai/ha or more, 3250 g ai/ha or more, 3300 g ai/ha or more, 3400 g ai/ha or more, 3500 g ai/ha or more, 3750 g ai/ha or more, 4000 g ai/ha or more, 4250 g ai/ha or more, 4500 g ai/ha or more, 4750 g ai/ha or more, 5000 g ai/ha or more, 5250 g ai/ha or more, 5500 g ai/ha or more, 5750 g ai/ha or more, 6000 g ai/ha or more, 6250 g ai/ha or more, 6500 g ai/ha or more, or 6750 g ai/ha or more; in an amount of 7000 g ai/ha or less, such as 6750 g ai/ha or less, 6500 g ai/ha or less, 6250 g ai/ha or less, 6000 g ai/ha or less, 5750 g ai/ha or less, 5500 g ai/ha or less, 5250 g ai/ha or less, 5000 g ai/ha or less, 4750 g ai/ha or less, 4500 g ai/ha or less, 4250 g ai/ha or less, 4000 g ai/ha or less, 3750 g ai/ha or less, 3500 g ai/ha or less, 3400 g ai/ha or less, 3300 g ai/ha or less, 3250 g ai/ha or less, 3200 g ai/ha or less, or 3100 g ai/ha or less; or in an amount ranging from any of the minimum values described above to any of the maximum values described above, such as 3000-7000 g ai/ha, 4500-6500 g ai/ha, 3100-3750 g ai/ha, 6000-7000 g ai/ha, 3750-5500 g ai/ha, 3300-4250 g ai/ha, 5000-6000 g ai/ha, 4250-5750 g ai/ha, 3250-5250 g ai/ha, 3500-4000 g ai/ha, or 3100-6750 g ai/ha.

### Cycloate

In some aspects, the FA/LSI herbicide can comprise cycloate or an agriculturally acceptable salt or ester thereof. Cycloate, shown below, is a thiocarbamate herbicide used for pre-plant incorporated control of annual and some perennial grass weeds, Cyperus spp., and some annual broad-leaved weeds in sugar beets, fodder beets, beetroot and spinach, at 3000-4000 g/ha. Cycloate, as well as methods of preparing cycloate, are known in the art. Its herbicidal activity is described, for example, in The Pesticide Manual, Seventeenth Edition, 2016.

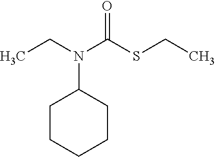

Cycloate can be applied to vegetation or an area adjacent the vegetation or applied to soil or water to prevent the emergence or growth of vegetation in an amount sufficient to induce a herbicidal effect. In some aspects, cycloate is applied to vegetation or an area adjacent the vegetation or applied to soil or water to prevent the emergence or growth of vegetation in an amount of 2500 g ai/ha or more, such as 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3750 g ai/ha or more, 4000 g ai/ha or more, or 4250 g ai/ha or more; in an amount of 4500 g ai/ha or less, such as 4250 g ai/ha or less, 4000 g ai/ha or less, 3750 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2700 g ai/ha or less, or 2600 g ai/ha or less; or in an amount ranging from any of the minimum values described above to any of the maximum values described above, such as 2500-4500 g ai/ha, 3200-4000 g ai/ha, 2800-3750 g ai/ha, 3600-4250 g ai/ha, 3750-4250 g ai/ha, 2900-4000 g ai/ha, 3250-4500 g ai/ha, 3100-4000 g ai/ha, 2500-3400 g ai/ha, or 2600-4250 g ai/ha.

### Dalapon

In some aspects, the FA/LSI herbicide can comprise dalapon or an agriculturally acceptable salt or ester thereof. Dalapon, shown below, is a thiocarbamate herbicide used for pre- and post-emergence control of annual and perennial grass weeds in non-crop land, e.g. embankments, roadside verges, industrial sites, railway tracks, irrigation channels, ditches, etc., pome fruit, stone fruit, bush fruit, citrus, nuts, olives, grapes, forestry, bananas, sugar cane, rhubarb, asparagus, potatoes, peas, soybeans, beets, oilseed rape, flax, maize, sorghum, coffee, tea, rubber, cotton and ornamental shrubs; control of reed, sedge, rush, halophyte and semi-aquatic grass weeds in water courses. Common agriculturally acceptable salts of dalapon include the magnesium and sodium salts thereof. Dalapon, as well as methods of preparing dalapon, are known in the art. Its herbicidal activity is described, for example, in The Pesticide Manual, Seventeenth Edition, 2016.

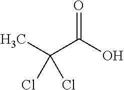

Dalapon can be applied to vegetation or an area adjacent the vegetation or applied to soil or water to prevent the emergence or growth of vegetation in an amount sufficient to induce a herbicidal effect. In some aspects, dalapon is applied to vegetation or an area adjacent the vegetation or applied to soil or water to prevent the emergence or growth of vegetation in an amount of 1500 g ai/ha or more, such as 1600 g ai/ha or more, 1700 g ai/ha or more, 1750 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3750 g ai/ha or more, 4000 g ai/ha or more, 4250 g ai/ha or more, 4500 g ai/ha or more, 4750 g ai/ha or more, 5000 g ai/ha or more, 5250 g ai/ha or more, 5500 g ai/ha or more, 5750 g ai/ha or more, 6000 g ai/ha or more, 6250 g ai/ha or more, 6500 g ai/ha or more, 6750 g ai/ha or more, 7000 g ai/ha or more, 7250 g ai/ha or more, 7500 g ai/ha or more, 7750 g ai/ha or more, 8000 g ai/ha or more, 8250 g ai/ha or more, 8500 g ai/ha or more, 8750 g ai/ha or more, 9000 g ai/ha or more, 9250 g ai/ha or more, 9500 g ai/ha or more, 9750 g ai/ha or more, 10 kg ai/ha or more, 10.5 kg ai/ha or more, 11 kg ai/ha or more, 11.5 kg ai/ha or more, 12 kg ai/ha or more, 12.5 kg ai/ha or more, 13 kg ai/ha or more, 13.5 kg ai/ha or more, 14 kg ai/ha or more, 14.5 kg ai/ha or more, 15 kg ai/ha or more, 15.5 kg ai/ha or more, 16 kg ai/ha or more, 16.5 kg ai/ha or more, 16.75 kg ai/ha or more, 17 kg ai/ha or more, 17.5 kg ai/ha or more, 18 kg ai/ha or more, 18.5 kg ai/ha or more, 19 kg ai/ha or more, 19.5 kg ai/ha or more, 20 kg ai/ha or more, or 20.5 kg ai/ha or more; in an amount of 21 kg ai/ha or less, such as 20.5 kg ai/ha or less, 20 kg ai/ha or less, 19.5 kg ai/ha or less, 19 kg ai/ha or less, 18.5 kg ai/ha or less, 18 kg ai/ha or less, 17.5 kg ai/ha or less, 17 kg ai/ha or less, 16.5 kg ai/ha or less, 16 kg ai/ha or less, 15.5 kg ai/ha or less, 15 kg ai/ha or less, 14.5 kg ai/ha or less, 14 kg ai/ha or less, 13.5 kg ai/ha or less, 13 kg ai/ha or less, 12.5 kg ai/ha or less, 12 kg ai/ha or less, 11.5 kg ai/ha or less, 11 kg ai/ha or less, 10.5 kg ai/ha, 10 kg ai/ha or less, 9750 g ai/ha or less, 9500 g ai/ha or less, 9250 g ai/ha or less, 9000 g ai/ha or less, 8750 g ai/ha or less, 8500 g ai/ha or less, 8250 g ai/ha or less, 8000 g ai/ha or less, 7750 g ai/ha or less, 7500 g ai/ha or less, 7250 g ai/ha or less, 7000 g ai/ha or less, 6750 g ai/ha or less, 6500 g ai/ha or less, 6250 g ai/ha or less, 6000 g ai/ha or less, 5750 g ai/ha or less, 5500 g ai/ha or less, 5250 g ai/ha or less, 5000 g ai/ha or less, 4750 g ai/ha or less, 4500 g ai/ha or less, 4250 g ai/ha or less, 4000 g ai/ha or less, 3750 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1750 g ai/ha or less, 1700 g ai/ha or less, or 1600 g ai/ha or less; or in an amount ranging from any of the minimum values described above to any of the maximum values described above, such as 1500 g-21 kg ai/ha, 7250-8500 g ai/ha, 14-20 kg ai/ha, 6500-8250 g ai/ha, 5-15 kg ai/ha, 11-14.5 kg ai/ha, 12.5-19 kg ai/ha, 7500 g-17 kg ai/ha, 1800-5250 g ai/ha, 4500-8500 g ai/ha, 6750-9750 g ai/ha, or 1600 g-20.5 kg ai/ha

### EPTC

In some aspects, the FA/LSI herbicide can comprise S-ethyl dipropylthiocarbamate, more commonly known as EPTC, or an agriculturally acceptable salt or ester thereof. EPTC, shown below, is a thiocarbamate herbicide used for pre-plant incorporated control of annual and perennial grass, especially couch grass, sedge, e.g. Cyperus spp. and some broad-leaved weeds in potatoes, beans, peas, forage legumes, beetroot, sugar beets, alfalfa, trefoil, clover, cotton, maize, flax, sweet potatoes, safflowers, sunflowers, strawberries, citrus, almonds, walnuts, ornamentals, pineapples and pine nurseries, at 4500-6700 g/ha, and in maize, with dichlormid safener. EPTC, as well as methods of preparing EPTC, are known in the art. Its herbicidal activity is described, for example, in The Pesticide Manual, Seventeenth Edition, 2016.

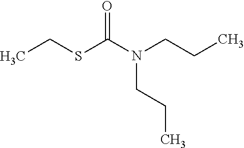

EPTC can be applied to vegetation or an area adjacent the vegetation or applied to soil or water to prevent the emergence or growth of vegetation in an amount sufficient to induce a herbicidal effect. In some aspects, EPTC is applied to vegetation or an area adjacent the vegetation or applied to soil or water to prevent the emergence or growth of vegetation in an amount of 1000 g ai/ha or more, such as 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1750 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3750 g ai/ha or more, 4000 g ai/ha or more, 4250 g ai/ha or more, 4500 g ai/ha or more, 4750 g ai/ha or more, 5000 g ai/ha or more, 5250 g ai/ha or more, 5500 g ai/ha or more, 5750 g ai/ha or more, 6000 g ai/ha or more, 6250 g ai/ha or more, 6500 g ai/ha or more, 6750 g ai/ha or more, 7000 g ai/ha or more, 7250 g ai/ha or more, 7500 g ai/ha or more, 7750 g ai/ha or more, 8000 g ai/ha or more, 8250 g ai/ha or more, 8500 g ai/ha or more, or 8750 g ai/ha or more; in an amount of 9000 g ai/ha or less, such as 8750 g ai/ha or less, 8500 g ai/ha or less, 8250 g ai/ha or less, 8000 g ai/ha or less, 7750 g ai/ha or less, 7500 g ai/ha or less, 7250 g ai/ha or less, 7000 g ai/ha or less, 6750 g ai/ha or less, 6500 g ai/ha or less, 6250 g ai/ha or less, 6000 g ai/ha or less, 5750 g ai/ha or less, 5500 g ai/ha or less, 5250 g ai/ha or less, 5000 g ai/ha or less, 4750 g ai/ha or less, 4500 g ai/ha or less, 4250 g ai/ha or less, 4000 g ai/ha or less, 3750 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, or 1050 g ai/ha or less; or in an amount ranging from any of the minimum values described above to any of the maximum values described above, such as 1000-9000 g ai/ha, 2200-6500 g ai/ha, 1450-3750 g ai/ha, 2600-7500 g ai/ha, 1050-4000 g ai/ha, 2100-4500 g ai/ha, 1900-5500 g ai/ha, 7250-8500 g ai/ha, 3300-6250 g ai/ha, 1500-3000 g ai/ha, 2500-8250 g ai/ha, or 1050-8750 g ai/ha.

### Esprocarb

In some aspects, the FA/LSI herbicide can comprise esprocarb, or an agriculturally acceptable salt or ester thereof. Esprocarb, shown below, is a thiocarbamate herbicide used for pre- and post-emergence control of annual weeds, e.g. Echinochloa spp., in paddy rice, at 1500-4000 g/ha. Esprocarb, as well as methods of preparing esprocarb, are known in the art. Its herbicidal activity is described, for example, in The Pesticide Manual, Seventeenth Edition, 2016.

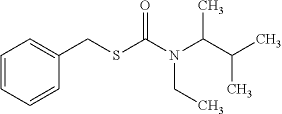 [SMILES: CCN(C(=O)SCc1ccccc1)C(C)C(C)C]

Esprocarb can be applied to vegetation or an area adjacent the vegetation or applied to soil or water to prevent the emergence or growth of vegetation in an amount sufficient to induce a herbicidal effect. In some aspects, esprocarb is applied to vegetation or an area adjacent the vegetation or applied to soil or water to prevent the emergence or growth of vegetation in an amount of 400 g ai/ha or more, such as 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1750 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, or 3750 g ai/ha or more; 4000 g ai/ha or less, such as 3750 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, or 410 g ai/ha or less; or in an amount ranging from any of the minimum values described above to any of the maximum values described above, such as 400-4000 g ai/ha, 2500-3500 g ai/ha, 600-2700 g ai/ha, 3100-4000 g ai/ha, 2000-3000 g ai/ha, 1800-3250 g ai/ha, 900-3400 g ai/ha, 1750-2200 g ai/ha, 2800-3750 g ai/ha, 550-1900 g ai/ha, 1700-3300 g ai/ha, or 410-3750 g ai/ha.

### Ethofumesate

In some aspects, the FA/LSI herbicide can comprise ethofumesate, or an agriculturally acceptable salt or ester thereof. Ethofumesate, shown below, is a benzofuran herbicide used for pre- and post-emergence control of grass and broad-leaved weeds in sugar and other beets, turf, ryegrass and other pasture grasses, at 300-2000 g/ha. Ethofumesate, as well as methods of preparing ethofumesate, are known in the art. Its herbicidal activity is described, for example, in The Pesticide Manual, Seventeenth Edition, 2016.

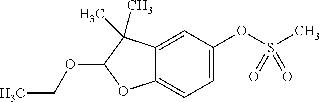 [SMILES: CCOC1Oc2ccc(OS(C)(=O)=O)cc2C1(C)C]

Ethofumesate can be applied to vegetation or an area adjacent the vegetation or applied to soil or water to prevent the emergence or growth of vegetation in an amount sufficient to induce a herbicidal effect. In some aspects, ethofumesate is applied to vegetation or an area adjacent the vegetation or applied to soil or water to prevent the emergence or growth of vegetation in an amount of 200 g ai/ha or more, such as 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1750 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, or 2900 g ai/ha or more; in an amount of 3000 g ai/ha or less, such as 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, or 210 g ai/ha or less; or in an amount ranging from any of the minimum values described above to any of the maximum values described above, such as 200-3000 g ai/ha, 950-2500 g ai/ha, 460-1750 g ai/ha, 600-2500 g ai/ha, 350-2200 g ai/ha, 310-450 g ai/ha, 500-2400 g ai/ha, 750-2200 g ai/ha, 380-1350 g ai/ha, 575-1300 g ai/ha, 440-2600 g ai/ha, or 210-2900 g ai/ha.

### Flupropanate

In some aspects, the FA/LSI herbicide can comprise flupropanate, or an agriculturally acceptable salt or ester thereof. Flupropanate, shown below, is a halogenated alkanoic acid herbicide used for post-emergence control of annual and perennial grass weeds, e.g., serrated tussock and Chilean needle grass, in pastures and in uncultivated land. One agriculturally acceptable salt of flupropanate is flupropanate-sodium. Flupropanate, as well as methods of preparing flupropanate, are known in the art. Its herbicidal activity is described, for example, in The Pesticide Manual, Seventeenth Edition, 2016.

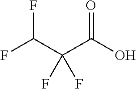

Flupropanate can be applied to vegetation or an area adjacent the vegetation or applied to soil or water to prevent the emergence or growth of vegetation in an amount sufficient to induce a herbicidal effect. In some aspects, flupropanate is applied to vegetation or an area adjacent the vegetation or applied to soil or water to prevent the emergence or growth of vegetation in an amount of 1000 g ai/ha or more, such as 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1750 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, or 2900 g ai/ha or more; in an amount of 3000 g ai/ha or less, such as 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, or 1050 g ai/ha or less; or in an amount ranging from any of the minimum values described above to any of the maximum values described above, such as 1000-3000 g ai/ha, 1150-2500 g ai/ha, 1400-1750 g ai/ha, 1600-2500 g ai/ha, 1050-2200 g ai/ha, 1350-2400 g ai/ha, 2500-2900 g ai/ha, 1750-2700 g ai/ha, 1300-1600 g ai/ha, 1800-2300 g ai/ha, or 1050-2900 g ai/ha.

### Molinate

In some aspects, the FA/LSI herbicide can comprise molinate, or an agriculturally acceptable salt or ester thereof. Molinate, shown below, is a thiocarbamate herbicide used for control of germinating broad-leaved and grass weeds, particularly Echinochloa spp. and Glyceria spp., in rice, at 2500-5000 g/ha, applied either before planting to water-seeded or shallow soil-seeded rice or post-flood, post-emergence in other types of rice culture. Molinate, as well as methods of preparing molinate, are known in the art. Its herbicidal activity is described, for example, in The Pesticide Manual, Seventeenth Edition, 2016.

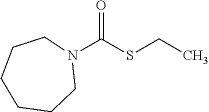

Molinate can be applied to vegetation or an area adjacent the vegetation or applied to soil or water to prevent the emergence or growth of vegetation in an amount sufficient to induce a herbicidal effect. In some aspects, molinate is applied to vegetation or an area adjacent the vegetation or applied to soil or water to prevent the emergence or growth of vegetation in an amount of 2500 g ai/ha or more, such as 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3750 g ai/ha or more, 4000 g ai/ha or more, 4250 g ai/ha or more, 4500 g ai/ha or more, or 4750 g ai/ha or more; in an amount of 5000 g ai/ha or less, such as 4750 g ai/ha or less, 4500 g ai/ha or less, 4250 g ai/ha or less, 4000 g ai/ha or less, 3750 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2700 g ai/ha or less, or 2600 g ai/ha or less; or in an amount ranging from any of the minimum values described above to any of the maximum values described above, such as 2500-5000 g ai/ha, 3200-4500 g ai/ha, 2600-3750 g ai/ha, 3000-3500 g ai/ha, 2800-3400 g ai/ha, 2700-3100 g ai/ha, 2900-4250 g ai/ha, 3750-4750 g ai/ha, 3300-4500 g ai/ha, 2500-3500 g ai/ha, or 2600-4750 g ai/ha.

### Orbencarb

In some aspects, the FA/LSI herbicide can comprise orbencarb, or an agriculturally acceptable salt or ester thereof. Orbencarb, shown below, is a thiocarbamate herbicide used for pre-emergence control of annual grass, except wild oats, and broad-leaved weeds in barley, wheat, rye, maize, soybeans, cotton and turf, at 4000-5000 g/ha. Orbencarb, as well as methods of preparing orbencarb, are known in the art. Its herbicidal activity is described, for example, in The Pesticide Manual, Seventeenth Edition, 2016.

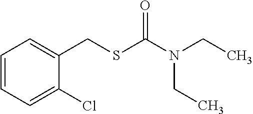 [SMILES: CCN(CC)C(=O)SCc1ccccc1Cl]

Orbencarb can be applied to vegetation or an area adjacent the vegetation or applied to soil or water to prevent the emergence or growth of vegetation in an amount sufficient to induce a herbicidal effect. In some aspects, orbencarb is applied to vegetation or an area adjacent the vegetation or applied to soil or water to prevent the emergence or growth of vegetation in an amount of 4000 g ai/ha or more, such as 4100 g ai/ha or more, 4200 g ai/ha or more, 4300 g ai/ha or more, 4400 g ai/ha or more, 4500 g ai/ha or more, 4600 g ai/ha or more, 4700 g ai/ha or more, 4800 g ai/ha or more, or 4900 g ai/ha or more; in an amount of 5000 g ai/ha or less, such as 4900 g ai/ha or less, 4800 g ai/ha or less, 4700 g ai/ha or less, 4600 g ai/ha or less, 4500 g ai/ha or less, 4400 g ai/ha or less, 4300 g ai/ha or less, 4200 g ai/ha or less, or 4100 g ai/ha or less; or in an amount ranging from any of the minimum values described above to any of the maximum values described above, such as 4000-5000 g ai/ha, 4200-4500 g ai/ha, 4600-4900 g ai/ha, 4100-4400 g ai/ha, 4500-5000 g ai/ha, or 4100-4900 g ai/ha.

### Prosulfocarb

In some aspects, the FA/LSI herbicide can comprise prosulfocarb, or an agriculturally acceptable salt or ester thereof. Prosulfocarb, shown below, is a thiocarbamate herbicide used for pre- and early post-emergence control of grass and broad-leaved weeds in winter wheat, winter barley and rye, at 3000-4000 g/ha. Prosulfocarb, as well as methods of preparing prosulfocarb, are known in the art. Its herbicidal activity is described, for example, in The Pesticide Manual, Seventeenth Edition, 2016.

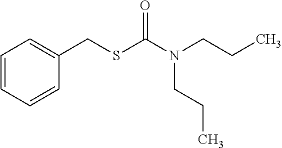 [SMILES: CCCN(CCC)C(=O)SCc1ccccc1]

Prosulfocarb can be applied to vegetation or an area adjacent the vegetation or applied to soil or water to prevent the emergence or growth of vegetation in an amount sufficient to induce a herbicidal effect. In some aspects, prosulfocarb is applied to vegetation or an area adjacent the vegetation or applied to soil or water to prevent the emergence or growth of vegetation in an amount of 250 g ai/ha or more, such as 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, 675 g ai/ha or more, 700 g ai/ha or more, 750 g ai/ha or more, 800 g ai/ha or more, 850 g ai/ha or more, 900 g ai/ha or more, 950 g ai/ha or more, 1000 g ai/ha or more, 1050 g ai/ha or more, 1100 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1750 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, or 3750 g ai/ha or more; in an amount of 4000 g ai/ha or less, such as 3750 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, 1100 g ai/ha or less, 1050 g ai/ha or less, 1000 g ai/ha or less, 950 g ai/ha or less, 900 g ai/ha or less, 850 g ai/ha or less, 800 g ai/ha or less, 750 g ai/ha or less, 700 g ai/ha or less, 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less; or in an amount ranging from any of the minimum values described above to any of the maximum values described above, such as 250-4000 g ai/ha, 1350-3500 g ai/ha, 1600-3750 g ai/ha, 800-2500 g ai/ha, 1250-2000 g ai/ha, 1100-3100 g ai/ha, 1900-2400 g ai/ha, 320-3200 g ai/ha, 450-850 g ai/ha, 2700-3400 g ai/ha, 1500-2900 g ai/ha, or 260-3750 g ai/ha.

### Thiobencarb

In some aspects, the FA/LSI herbicide can comprise thiobencarb, or an agriculturally acceptable salt or ester thereof. Thiobencarb, shown below, is a thiocarbamate herbicide used for pre- to early post-emergence control of Echinochloa, Leptochloa and Cyperus spp. and other grass and annual broad-leaved weeds in direct-seeded and transplanted rice, at 1500-7500 g/ha. Thiobencarb, as well as methods of preparing thiobencarb, are known in the art. Its herbicidal activity is described, for example, in The Pesticide Manual, Seventeenth Edition, 2016.

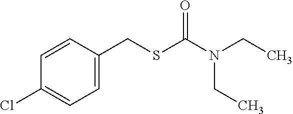 [SMILES: CCN(CC)C(=O)SCc1ccc(Cl)cc1]

Thiobencarb can be applied to vegetation or an area adjacent the vegetation or applied to soil or water to prevent the emergence or growth of vegetation in an amount sufficient to induce a herbicidal effect. In some aspects, thiobencarb is applied to vegetation or an area adjacent the vegetation or applied to soil or water to prevent the emergence or growth of vegetation in an amount of 1100 g ai/ha or more, such as 1120 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1600 g ai/ha or more, 1700 g ai/ha or more, 1750 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, 2900 g ai/ha or more, 3000 g ai/ha or more, 3100 g ai/ha or more, 3200 g ai/ha or more, 3250 g ai/ha or more, 3300 g ai/ha or more, 3400 g ai/ha or more, 3500 g ai/ha or more, 3750 g ai/ha or more, 4000 g ai/ha or more, 4250 g ai/ha or more, 4500 g ai/ha or more, 4750 g ai/ha or more, 5000 g ai/ha or more, 5250 g ai/ha or more, 5500 g ai/ha or more, 5750 g ai/ha or more, 6000 g ai/ha or more, 6250 g ai/ha or more, 6500 g ai/ha or more, 6750 g ai/ha or more, 7000 g ai/ha or more, or 7250 g ai/ha or more; in an amount of 7500 g ai/ha or less, such as 7250 g ai/ha or less, 7000 g ai/ha or less, 6750 g ai/ha or less, 6500 g ai/ha or less, 6250 g ai/ha or less, 6000 g ai/ha or less, 5750 g ai/ha or less, 5500 g ai/ha or less, 5250 g ai/ha or less, 5000 g ai/ha or less, 4750 g ai/ha or less, 4500 g ai/ha or less, 4250 g ai/ha or less, 4000 g ai/ha or less, 3750 g ai/ha or less, 3500 g ai/ha or less, 3400 g ai/ha or less, 3300 g ai/ha or less, 3250 g ai/ha or less, 3200 g ai/ha or less, 3100 g ai/ha or less, 3000 g ai/ha or less, 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1750 g ai/ha or less, 1700 g ai/ha or less, 1600 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, or 1120 g ai/ha or less; or in an amount ranging from any of the minimum values described above to any of the maximum values described above, such as 1100-7500 g ai/ha, 3750-6500 g ai/ha, 1250-3400 g ai/ha, 2600-5500 g ai/ha, 1800-5000 g ai/ha, 2100-6250 g ai/ha, 1900-4000 g ai/ha, 2700-4500 g ai/ha, 2000-5250 g ai/ha, 6500-7500 g ai/ha, 1300-4000 g ai/ha, or 1120-7250 g ai/ha.

### Tiocarbazil

In some aspects, the FA/LSI herbicide can comprise tiocarbazil, or an agriculturally acceptable salt or ester thereof. Tiocarbazil, shown below, is a thiocarbamate herbicide used for pre- or post-emergence control of grass weeds, e.g. Echinochloa spp. and Lolium perenne, and sedge weeds, e.g. Cyperus spp., in rice paddies, at 4000 g/ha. Tiocarbazil, as well as methods of preparing tiocarbazil, are known in the art. Its herbicidal activity is described, for example, in The Pesticide Manual, Seventeenth Edition, 2016.

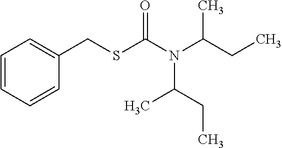 [SMILES: CCC(C)N(C(=O)SCc1ccccc1)C(C)CC]

### Tri-Allate

In some aspects, the FA/LSI herbicide can comprise tri-allate, or an agriculturally acceptable salt or ester thereof. Tri-allate, shown below, is a thiocarbamate herbicide used for pre-plant or post-plant incorporated control of wild oats and some annual grass weeds in wheat, barley, rye, field beans, peas, lentils, beets, oilseed rape, maize, flax, alfalfa, clover, vetches, sainfoin, safflowers, sunflowers and vegetables, at 1120-1680 g/ha. Tri-allate, as well as methods of preparing tri-allate, are known in the art. Its herbicidal activity is described, for example, in The Pesticide Manual, Seventeenth Edition, 2016.

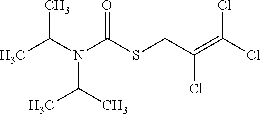 [SMILES: CC(C)N(C(=O)SCC(Cl)=C(Cl)Cl)C(C)C]

Tri-allate can be applied to vegetation or an area adjacent the vegetation or applied to soil or water to prevent the emergence or growth of vegetation in an amount sufficient to induce a herbicidal effect. In some aspects, tri-allate is applied to vegetation or an area adjacent the vegetation or applied to soil or water to prevent the emergence or growth of vegetation in an amount of 1100 g ai/ha or more, such as 1120 g ai/ha or more, 1150 g ai/ha or more, 1200 g ai/ha or more, 1250 g ai/ha or more, 1300 g ai/ha or more, 1350 g ai/ha or more, 1400 g ai/ha or more, 1450 g ai/ha or more, 1500 g ai/ha or more, 1550 g ai/ha or more, 1600 g ai/ha or more, 1650 g ai/ha or more, or 1680 g ai/ha or more; in an amount of 1700 g ai/ha or less, such as 1680 g ai/ha or less, 1650 g ai/ha or less, 1600 g ai/ha or less, 1550 g ai/ha or less, 1500 g ai/ha or less, 1450 g ai/ha or less, 1400 g ai/ha or less, 1350 g ai/ha or less, 1300 g ai/ha or less, 1250 g ai/ha or less, 1200 g ai/ha or less, 1150 g ai/ha or less, or 1120 g ai/ha or less; or in an amount ranging from any of the minimum values described above to any of the maximum values described above, such as 1100-1700 g ai/ha, 1250-1600 g ai/ha, 1550-1650 g ai/ha, 1200-1500 g ai/ha, 1150-1300 g ai/ha, 1120-1400 g ai/ha, 1450-1700 g ai/ha, 1100-1350 g ai/ha, or 1120-1680 g ai/ha.

### Vernolate

In some aspects, the FA/LSI herbicide can comprise vernolate, or an agriculturally acceptable salt or ester thereof. Vernolate, shown below, is a thiocarbamate herbicide used for pre-plant or post-plant incorporated control of wild oats and some annual grass weeds in wheat, barley, rye, field beans, peas, lentils, beets, oilseed rape, maize, flax, alfalfa, clover, vetches, sainfoin, safflowers, sunflowers and vegetables, at 1120-1680 g/ha. Vernolate, as well as methods of preparing vernolate, are known in the art. Its herbicidal activity is described, for example, in The Pesticide Manual, Seventeenth Edition, 2016.

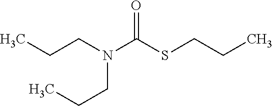

Vernolate can be applied to vegetation or an area adjacent the vegetation or applied to soil or water to prevent the emergence or growth of vegetation in an amount sufficient to induce a herbicidal effect. In some aspects, vernolate is applied to vegetation or an area adjacent the vegetation or applied to soil or water to prevent the emergence or growth of vegetation in an amount of 1500 g ai/ha or more, such as 1600 g ai/ha or more, 1700 g ai/ha or more, 1750 g ai/ha or more, 1800 g ai/ha or more, 1900 g ai/ha or more, 2000 g ai/ha or more, 2100 g ai/ha or more, 2200 g ai/ha or more, 2300 g ai/ha or more, 2400 g ai/ha or more, 2500 g ai/ha or more, 2600 g ai/ha or more, 2700 g ai/ha or more, 2800 g ai/ha or more, or 2900 g ai/ha or more; in an amount of 3000 g ai/ha or less, such as 2900 g ai/ha or less, 2800 g ai/ha or less, 2700 g ai/ha or less, 2600 g ai/ha or less, 2500 g ai/ha or less, 2400 g ai/ha or less, 2300 g ai/ha or less, 2200 g ai/ha or less, 2100 g ai/ha or less, 2000 g ai/ha or less, 1900 g ai/ha or less, 1800 g ai/ha or less, 1750 g ai/ha or less, 1700 g ai/ha or less, or 1600 g ai/ha or less; or in an amount ranging from any of the minimum values described above to any of the maximum values described above, such as 1500-3000 g ai/ha, 1800-2500 g ai/ha, 1700-1900 g ai/ha, 1600-2800 g ai/ha, 1750-2200 g ai/ha, 2350-2900 g ai/ha, 1600-2100 g ai/ha, 1750-2700 g ai/ha, 2100-2600 g ai/ha, 1800-2300 g ai/ha, or 1600-2900 g ai/ha.

### Other Herbicides

### Broclozone

Broclozone, the ISO name for 2-[(2-bromo-4-chlorophenyl)methyl]-4,4-dimethyl-3-isoxazolidinone (CAS No. 2766607-82-1), is a DOXP-inhibiting oxazole herbicide from Qingdao KingAgroot Chemical Compound Co. and is disclosed in WO202302027. Broclozone may be used with foliar and/or soil applications, and may be used on crops such as wheat, soybeans, peanuts, oilseed rape, cabbage, rice, cotton, garlic, and watermelon.

Broclozone may be applied to vegetation or an area adjacent the vegetation or applied to soil or water to prevent the emergence or growth of vegetation in an amount of 300 g ai/ha or more, such as 310 g ai/ha or more, 320 g ai/ha or more, 330 g ai/ha or more, 340 g ai/ha or more, 350 g ai/ha or more, 360 g ai/ha or more, 370 g ai/ha or more, 380 g ai/ha or more, 390 g ai/ha or more, 400 g ai/ha or more, 410 g ai/ha or more, 420 g ai/ha or more, 430 g ai/ha or more, 440 g ai/ha or more, 450 g ai/ha or more, 460 g ai/ha or more, 480 g ai/ha or more, 500 g ai/ha or more, 520 g ai/ha or more, 540 g ai/ha or more, 550 g ai/ha or more, 560 g ai/ha or more, 580 g ai/ha or more, 600 g ai/ha or more, 620 g ai/ha or more, 640 g ai/ha or more, 650 g ai/ha or more, 660 g ai/ha or more, 680 g ai/ha or more, 700 g ai/ha or more, 720 g ai/ha or more, or 740 g ai/ha or more; in an amount of 750 g ai/ha or less, such as 740 g ai/ha or less, 720 g ai/ha or less, 700 g ai/ha or less, 680 g ai/ha or less, 660 g ai/ha or less, 650 g ai/ha or less, 640 g ai/ha or less, 620 g ai/ha or less, 600 g ai/ha or less, 580 g ai/ha or less, 560 g ai/ha or less, 550 g ai/ha or less, 540 g ai/ha or less, 520 g ai/ha or less, 500 g ai/ha or less, 480 g ai/ha or less, 460 g ai/ha or less, 450 g ai/ha or less, 440 g ai/ha or less, 430 g ai/ha or less, 420 g ai/ha or less, 410 g ai/ha or less, 400 g ai/ha or less, 390 g ai/ha or less, 380 g ai/ha or less, 370 g ai/ha or less, 360 g ai/ha or less, 350 g ai/ha or less, 340 g ai/ha or less, 330 g ai/ha or less, 320 g ai/ha or less, or 310 g ai/ha or less; or in an amount ranging from any of the minimum values described above to any of the maximum values described above, such as 300-750 g ai/ha, 320-580 g ai/ha, 330-660 g ai/ha, 410-650 g ai/ha, 340-700 g ai/ha, 450-520 g ai/ha, 500-750 g ai/ha, 400-600 g ai/ha, 350-720 g ai/ha, or 420-740 g ai/ha.

### Flufenoximacil

Flufenoximacil, the ISO name for methyl (2R)-2-[[(E)-[[2-chloro-5-[3,6-dihydro-3-methyl-2,6-dioxo-4-(trifluoromethyl)-1 (2H)-pyrimidinyl]-4-fluorophenyl]methylene]amino]oxy] propanoate (CAS No. 2759011-88-4), is a phenyluracil herbicide for non-selective weed control from Qingdao KingAgroot Chemical Compound Co. and is described in U.S Patent Application Publication No. 2023/0111310.

Flufenoximacil is applied to vegetation or an area adjacent the vegetation or applied to soil or water to prevent the emergence or growth of vegetation in an amount of 1 g ai/ha or more, such as 2 g ai/ha or more, 2.5 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 110 g ai/ha or more, 120 g ai/ha or more, 125 g ai/ha or more, 130 g ai/ha or more, 140 g ai/ha or more, 142.5 g ai/ha or more, 145 g ai/ha or more, 147 g ai/ha or more, or 149 g ai/ha or more; in an amount of 150 g ai/ha or less, such as 149 g ai/ha or less, 147.5 g ai/ha or less, 145 g ai/ha or less, 142.5 g ai/ha or less, 140 g ai/ha or less, 130 g ai/ha or less, 125 g ai/ha or less, 120 g ai/ha or less, 110 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, 2.5 g ai/ha or less, or 2 g ai/ha or less; or in an amount ranging from any of the minimum values described above to any of the maximum values described above, such as 1-150 g ai/ha, 2-130 g ai/ha, 2.5-45 g ai/ha, 3-70 g ai/ha, 4-147 g ai/ha, 5-100 g ai/ha, 6-75 g ai/ha, 7-50 g ai/ha, 10-149 g ai/ha, 20-120 g ai/ha, 25-90 g ai/ha, 30-60 g ai/ha, 50-150 g ai/ha, 60-130 g ai/ha, or 100-145 g ai/ha.

### Flusulfinam

Flusulfinam, the ISO name for 2-fluoro-N-(5-methyl-1,3,4-oxadiazol-2-yl)-3-(propylsulfinyl)-4-(trifluoromethyl)benzamide (the (R)-enantiomer), is an amide herbicide from Qingdao KingAgroot Chemical Compound Co., and is described in US Patent Application Publication No. 2023/0041563.

Flusulfinam is effective in controlling weeds commonly found around rice crops (such as indica rice, japonica rice), gramineous weed (such as rice barnyardgrass, Qianjinjin, barnyard grass, horse tang, Setaria), broad-leaved weeds (such as gazania, velvetleaf, amaranth, chickweed), and sedge weeds (such as sedge, fireflies).

Flusulfinam is applied to vegetation or an area adjacent the vegetation or applied to soil or water to prevent the emergence or growth of vegetation in an amount of 1 g ai/ha or more, such as 2 g ai/ha or more, 2.5 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 110 g ai/ha or more, 120 g ai/ha or more, 125 g ai/ha or more, 130 g ai/ha or more, 140 g ai/ha or more, 142.5 g ai/ha or more, 145 g ai/ha or more, 147 g ai/ha or more, or 149 g ai/ha or more; in an amount of 150 g ai/ha or less, such as 149 g ai/ha or less, 147.5 g ai/ha or less, 145 g ai/ha or less, 142.5 g ai/ha or less, 140 g ai/ha or less, 130 g ai/ha or less, 125 g ai/ha or less, 120 g ai/ha or less, 110 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, 2.5 g ai/ha or less, or 2 g ai/ha or less; or in an amount ranging from any of the minimum values described above to any of the maximum values described above, such as 1-150 g ai/ha, 2-130 g ai/ha, 2.5-45 g ai/ha, 3-70 g ai/ha, 4-147 g ai/ha, 5-100 g ai/ha, 6-75 g ai/ha, 7-50 g ai/ha, 10-149 g ai/ha, 20-120 g ai/ha, 25-90 g ai/ha, 30-60 g ai/ha, 50-150 g ai/ha, 60-130 g ai/ha, or 100-145 g ai/ha.

### Icafolin

Icafolin, the ISO name for (2RS,4RS)-4-({[(5S)-3-(3,5-difluorophenyl)-5-vinyl-4,5-dihydroisoxazol-5-yl]carbonyl}amino)tetrahydrofuran-2-carboxylic acid (including combinations of the (2S,4S), (2R,4R) isomers), is an isoxazole herbicide from Bayer AG (CAS Reg. No. 2749998-21-6) and is a broad-spectrum herbicide for the control of mono dicotyledonous annual weeds, such as Abutilon theophrasti, Alopecurus myosuroides, Amaranthus retroflexus, Avena fatua, Polygonum convolvulus, Setaria viridis, Veronica persica. Icafolin esters and salts activities are disclosed in International PCT Application Publication Nos. WO2018228985 and WO2018228986.

Icafolin can be applied pre-emergence or post-emergence to control various weeds, such as butilon theophrasti, Alopecurus myosuroides, Amaranthus retroflexus, Avena fatua, Echinochloca crus, during preemergence treatment. gallium, Hordeum murinum, Lolium rigidum, Pharitis purpurea, Polygonum convolvulus, Setaria viridis, Stellaria media, Veronica persica and Viola tricolor.

Icafolin is applied to vegetation or an area adjacent the vegetation or applied to soil or water to prevent the emergence or growth of vegetation in an amount of 1 g ai/ha or more, such as 2 g ai/ha or more, 2.5 g ai/ha or more, 3 g ai/ha or more, 4 g ai/ha or more, 5 g ai/ha or more, 6 g ai/ha or more, 7 g ai/ha or more, 8 g ai/ha or more, 9 g ai/ha or more, 10 g ai/ha or more, 12.5 g ai/ha or more, 15 g ai/ha or more, 17.5 g ai/ha or more, 20 g ai/ha or more, 25 g ai/ha or more, 30 g ai/ha or more, 35 g ai/ha or more, 40 g ai/ha or more, 45 g ai/ha or more, 50 g ai/ha or more, 60 g ai/ha or more, 70 g ai/ha or more, 75 g ai/ha or more, 80 g ai/ha or more, 90 g ai/ha or more, 100 g ai/ha or more, 110 g ai/ha or more, 120 g ai/ha or more, 125 g ai/ha or more, 130 g ai/ha or more, 140 g ai/ha or more, 142.5 g ai/ha or more, 145 g ai/ha or more, 147 g ai/ha or more, or 149 g ai/ha or more; in an amount of 150 g ai/ha or less, such as 149 g ai/ha or less, 147.5 g ai/ha or less, 145 g ai/ha or less, 142.5 g ai/ha or less, 140 g ai/ha or less, 130 g ai/ha or less, 125 g ai/ha or less, 120 g ai/ha or less, 110 g ai/ha or less, 100 g ai/ha or less, 90 g ai/ha or less, 80 g ai/ha or less, 75 g ai/ha or less, 70 g ai/ha or less, 60 g ai/ha or less, 50 g ai/ha or less, 45 g ai/ha or less, 40 g ai/ha or less, 35 g ai/ha or less, 30 g ai/ha or less, 25 g ai/ha or less, 20 g ai/ha or less, 17.5 g ai/ha or less, 15 g ai/ha or less, 12.5 g ai/ha or less, 10 g ai/ha or less, 9 g ai/ha or less, 8 g ai/ha or less, 7 g ai/ha or less, 6 g ai/ha or less, 5 g ai/ha or less, 4 g ai/ha or less, 3 g ai/ha or less, 2.5 g ai/ha or less, or 2 g ai/ha or less; or in an amount ranging from any of the minimum values described above to any of the maximum values described above, such as 1-150 g ai/ha, 2-130 g ai/ha, 2.5-45 g ai/ha, 3-70 g ai/ha, 4-147 g ai/ha, 5-100 g ai/ha, 6-75 g ai/ha, 7-50 g ai/ha, 10-149 g ai/ha, 20-120 g ai/ha, 25-90 g ai/ha, 30-60 g ai/ha, 50-150 g ai/ha, 60-130 g ai/ha, 100-145 g ai/ha, or preferably 30-80 g ai/ha.

### Iptriazopyrid

Iptriazopyrid, the ISO name for 3-[[(1-methylethyl)sulfonyl]methyl]-N-(5-methyl-1,3,4-oxadiazol-2-yl)-5-(trifluoromethyl)-1,2,4-triazolo[4,3-a]pyridine-8-carboxamide, is an amide herbicide from Nissan Chemical Corp. (CAS Reg. No. 1994348-72-9) and is disclosed in International PCT Application Publication Nos. WO2014192936 and WO2020189576 (polymorphic crystal forms of Iptriazopyrid may be used as an herbicide for paddy fields in any of the treatment methods of soil treatment and foliage treatment under flooding. In some embodiments, can be preferably used in a treatment method by foliar treatment when in mixtures of the disclosed compounds. For example, iptriazopyrid may also be used with crops such as corn, rice, wheat, barley, lime tree, embaku, sorghum, cotton, soybean, peanut, buckwheat, tensai, rapeseed, sunflower, sugar cane, tobacco and the like.

Iptriazopyrid may be applied to vegetation or an area adjacent the vegetation or applied to soil or water to prevent the emergence or growth of vegetation in an amount of 50 g ai/ha or more, such as 55 g ai/ha or more, 60 g ai/ha or more, 65 g ai/ha or more, 70 g ai/ha or more, 75 g ai/ha or more, 80 g ai/ha or more, 90 g ai/ha or more, 95 g ai/ha or more, 100 g ai/ha or more, 110 g ai/ha or more, 120 g ai/ha or more, 130 g ai/ha or more, 140 g ai/ha or more, 150 g ai/ha or more, 160 g ai/ha or more, 170 g ai/ha or more, 180 g ai/ha or more, 190 g ai/ha or more, 200 g ai/ha or more, 220 g ai/ha or more, 240 g ai/ha or more, 260 g ai/ha or more, 280 g ai/ha or more, 300 g ai/ha or more, 320 g ai/ha or more, 340 g ai/ha or more, 360 g ai/ha or more, 380 g ai/ha or more, 400 g ai/ha or more, 420 g ai/ha or more, 440 g ai/ha or more, 460 g ai/ha or more, 480 g ai/ha or more, 500 g ai/ha or more, 520 g ai/ha or more, 540 g ai/ha or more, 560 g ai/ha or more, 580 g ai/ha or more, 600 g ai/ha or more, 620 g ai/ha or more, 640 g ai/ha or more, 650 g ai/ha or more, 660 g ai/ha or more, 670 g ai/ha or more, 680 g ai/ha or more, or 690 g ai/ha or more; in an amount of 700 g ai/ha or less, such as 690 g ai/ha or less, 680 g ai/ha or less, 670 g ai/ha or less, 660 g ai/ha or less, 650 g ai/ha or less, 640 g ai/ha or less, 620 g ai/ha or less, 600 g ai/ha or less, 580 g ai/ha or less, 560 g ai/ha or less, 540 g ai/ha or less, 520 g ai/ha or less, 500 g ai/ha or less, 480 g ai/ha or less, 460 g ai/ha or less, 440 g ai/ha or less, 420 g ai/ha or less, 400 g ai/ha or less, 380 g ai/ha or less, 360 g ai/ha or less, 340 g ai/ha or less, 320 g ai/ha or less, 300 g ai/ha or less, 280 g ai/ha or less, 260 g ai/ha or less, 240 g ai/ha or less, 220 g ai/ha or less, 200 g ai/ha or less, 190 g ai/ha or less, 180 g ai/ha or less, 170 g ai/ha or less, 160 g ai/ha or less, 150 g ai/ha or less, 140 g ai/ha or less, 130 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, or 55 g ai/ha or less; or in an amount ranging from any of the minimum values described above to any of the maximum values described above, such as 50-700 g ai/ha, 55-400 g ai/ha, 60-650 g ai/ha, 75-520 g ai/ha, 80-400 g ai/ha, 85-500 g ai/ha, 90-220 g ai/ha, 100-700 g ai/ha, 100-500 g ai/ha, 110-640 g ai/ha, 120-420 g ai/ha, 120-500 g ai/ha, 140-670 g ai/ha, or 150-580 g ai/ha.

### Metproxybicyclone

Metproxybicyclone, the ISO name for the racemic mixture (1RS,5SR)-3-[2-methoxy-4-(prop-1-yn-1-yl)phenyl]-4-oxobicyclo[3.2.1]oct-2-en-2-yl methyl carbonate, available from Syngenta Crop Protection (CAS Reg. No. 1848207-89-5) and is described in International PCT Application Publication No. WO2015197468.

Metproxybicyclone is applied to vegetation or an area adjacent the vegetation or applied to soil or water to prevent the emergence or growth of vegetation in an amount of 15 g ai/ha or more, such as 20 g ai/ha or more, 25 g ai/ha or more, 30 g ai/ha or more, 35 g ai/ha or more, 40 g ai/ha or more, 45 g ai/ha or more, 50 g ai/ha or more, 55 g ai/ha or more, 60 g ai/ha or more, 65 g ai/ha or more, 70 g ai/ha or more, 75 g ai/ha or more, 80 g ai/ha or more, 85 g ai/ha or more, 90 g ai/ha or more, 95 g ai/ha or more, 100 g ai/ha or more, 110 g ai/ha or more, 120 g ai/ha or more, 125 g ai/ha or more, 130 g ai/ha or more, 140 g ai/ha or more, 150 g ai/ha or more, 160 g ai/ha or more, 170 g ai/ha or more, 175 g ai/ha or more, 180 g ai/ha or more, 190 g ai/ha or more, 200 g ai/ha or more, 210 g ai/ha or more, 220 g ai/ha or more, 225 g ai/ha or more, 230 g ai/ha or more, 240 g ai/ha or more, 250 g ai/ha or more, 260 g ai/ha or more, 270 g ai/ha or more, 275 g ai/ha or more, 280 g ai/ha or more, 290 g ai/ha or more, 300 g ai/ha or more, 310 g ai/ha or more, 320 g ai/ha or more, 325 g ai/ha or more, 330 g ai/ha or more, 340 g ai/ha or more, 350 g ai/ha or more, 360 g ai/ha or more, 370 g ai/ha or more, 375 g ai/ha or more, 380 g ai/ha or more, 390 g ai/ha or more, 400 g ai/ha or more, 410 g ai/ha or more, 420 g ai/ha or more, 425 g ai/ha or more, 430 g ai/ha or more, 440 g ai/ha or more, 450 g ai/ha or more, 460 g ai/ha or more, 470 g ai/ha or more, 475 g ai/ha or more, 480 g ai/ha or more, 490 g ai/ha or more, 500 g ai/ha or more, 525 g ai/ha or more, 550 g ai/ha or more, 575 g ai/ha or more, 600 g ai/ha or more, 625 g ai/ha or more, 650 g ai/ha or more, or 675 g ai/ha or more; in an amount of 700 g ai/ha or less, such as 675 g ai/ha or less, 650 g ai/ha or less, 625 g ai/ha or less, 600 g ai/ha or less, 575 g ai/ha or less, 550 g ai/ha or less, 525 g ai/ha or less, 500 g ai/ha or less, 490 g ai/ha or less, 480 g ai/ha or less, 475 g ai/ha or less, 470 g ai/ha or less, 460 g ai/ha or less, 450 g ai/ha or less, 440 g ai/ha or less, 430 g ai/ha or less, 425 g ai/ha or less, 420 g ai/ha or less, 410 g ai/ha or less, 400 g ai/ha or less, 390 g ai/ha or less, 380 g ai/ha or less, 375 g ai/ha or less, 370 g ai/ha or less, 360 g ai/ha or less, 350 g ai/ha or less, 340 g ai/ha or less, 330 g ai/ha or less, 325 g ai/ha or less, 320 g ai/ha or less, 310 g ai/ha or less, 300 g ai/ha or less, 290 g ai/ha or less, 280 g ai/ha or less, 275 g ai/ha or less, 270 g ai/ha or less, 260 g ai/ha or less, 250 g ai/ha or less, 240 g ai/ha or less, 230 g ai/ha or less, 225 g ai/ha or less, 220 g ai/ha or less, 210 g ai/ha or less, 200 g ai/ha or less, 190 g ai/ha or less, 180 g ai/ha or less, 175 g ai/ha or less, 170 g ai/ha or less, 160 g ai/ha or less, 150 g ai/ha or less, 140 g ai/ha or less, 130 g ai/ha or less, 125 g ai/ha or less, 120 g ai/ha or less, 110 g ai/ha or less, 100 g ai/ha or less, 95 g ai/ha or less, 90 g ai/ha or less, 85 g ai/ha or less, 80 g ai/ha or less, 75 g ai/ha or less, 70 g ai/ha or less, 65 g ai/ha or less, 60 g ai/ha or less, 55 g ai/ha or less, 50 g ai/ha or less, 45 g ai/ha or less, 40 g ai/ha or less, 35 g ai/ha or less, 30 g ai/ha or less, 25 g ai/ha or less, or 20 g ai/ha or less; or in an amount ranging from any of the minimum values described above to any of the maximum values described above, such as 15-700 g ai/ha, 45-650 g ai/ha, 30-375 g ai/ha, 160-250 g ai/ha, 220-550 g ai/ha, 110-450 g ai/ha, 290-490 g ai/ha, 70-430 g ai/ha, 300-525 g ai/ha, 500-650 g ai/ha, 340-575 g ai/ha, or 20-675 g ai/ha.

### Exemplification of Mixture Herbicidal Activity

The following Table lists the growth stages of the plants at the time of application for the results exemplified in this application:

TABLE 5
Growth Stages of Plants For Results Exemplified

| | Bayer | | Plant |
|---|---|---|---|
| Species | Code | Leaf Stage | Height (cm) |
| Corn | ZEAMX | 3 to 4 | 20 to 25 |
| Common Lambsquarters | CHEAL | 6 to 8 | 8 to 11 |
| Velvetleaf | ABUTH | 4 to 6 | 6 to 9 |
| Tall Waterhemp | AMATA | 4 to 6 | 7 to 10 |
| Horseweed | ERICA | 12 to 15 rosette | 10 to 13 |
| | | | (diameter) |

All 2,4-D pro herbicide analogs and trifludimaxazin were applied in their technical form. Appropriate amounts of the technical grade materials were dissolved first in solvent 1, a mixture of 97.3% acetone:dimethyl sulfoxide (DMSO). The appropriate amount of 2,4-D pro-herbicide required to spray all treatments in each trial was weighed out and dissolved in solvent 1 to create a stock solution. If samples did not readily dissolve, the sample bottles were placed in hot water and sonicated for 20 minutes. Additionally, the samples were kept in hot water until sprayed. This resulted in the test material being thoroughly dissolved. Aliquots of the stock solution were measured out to provide the quantity needed for each treatment on an acid equivalent basis for all 2,4-D pro-herbicide concepts and glyphosate. The volume of stock solution and amount of any mixing partner was calculated and subtracted from 30. This difference was added to the spray bottle with solvent mixture 2, a mix of acetone, water, isopropyl alcohol, DMSO, Agri-Dex® crop oil concentrate (a heavy range, paraffin-based petroleum oil and nonionic emulsifiers commercially available from Rosecare) and nonionic Tergitol 1557 in a 40.3:45.3:11.9:1.3:1.0:0.2% v/v ratio. All percentages are listed on a volume basis. Lastly the required amount of mixing partner was added to the spray bottle. Collectively these additions resulted in a mix size of 30 ml.

The following mixing partners used in these trials were applied in their formulated form: saflufenacil under the tradename SHARPEN®, 341.5 g ai/L suspension concentrate (SC), sulfentrazone under the tradename SPRATAN® 4F, 480 g ai/L suspension concentrate (SC), glyphoste-K salt under the tradename ROUNDUP POWERMAX® 3, 575 g ae/L soluble liquid (SL), glufosinate under the tradename LIBERTY® 280 SL, 280 g ai/L soluble liquid (SL), diclosulam under the tradename STRONGARM®, 840 g ai/KG water dispersible granule (WDG), Arylex under the tradename ELEVORE®, 68.5 g ai/L soluble liquid (SL), mesotrione under the tradename CALLISTO®, 420 g ai/L suspension concentrate (SC).

Appropriate amounts of all liquid formulated herbicides were measured out and added directly to the spray bottle. Diclosulam was formulated as a WDG, and the amount required for all treatments in a trial was measured out and dissolved in water to create a stock solution. Aliquots of the stock solution were measured out to provide the quantity needed for each treatment.

Plants were grown in a soil media composed of 90% by volume Promix BX (Premier Tech Horticulture, Quakertown, PA) and 10% Profile Greens Grade (Profile Products LLC, Buffalo Grove, IL). Promix BX contains approximately 83% Sphagnum Peat Moss, 13% Perlite, 5% vermiculite, and proprietary amounts of limestone, starter fertilizer, and wetting agent. Profile Greens Grade is a sand-sized granule formed from calcining illite and montmorillonite clay. Several seeds of each species were planted in 10 cm square pots and top watered twice daily. Plant material was propagated in a warm greenhouse with temperature ranging from 25 to 28° C. and 50 to 60% relative humidity at the Corteva Global Headquarters (Indianapolis, Indiana, USA). Natural light was supplemented with 1000-watt metal halide overhead lamps with an average illumination of 500 μE m⁻² s⁻¹ photosynthetic active radiation for 16 consecutive hours each day. Plants were top-watered prior to herbicide application and sub-watered after herbicide application.

Herbicide applications were made to 3 replicates of each plant species at the 3 to 8 leaf stage, depending on the species. Treatments were applied with a research track-sprayer (Generation III Research Sprayer manufactured by DeVries Manufacturing in Hollandale, MN, USA) calibrated to deliver a spray volume of 200 L/ha. The track-sprayer was fitted with an 8003E nozzle from Spray Systems Company (North Avenue and Schmale Road, P.O. Box 7900, Wheaton, IL 60187), and used a spray pressure of 276 kPa pressure and a speed of 3.1 km/h. The nozzle height was 46 cm above the plant canopy. The experimental design used for the trials was a randomized complete block with 3 replications per treatment. After application, plants were placed on carts according to the randomization and returned to the greenhouse. Plants were sub-watered after treatment with Indianapolis city water or fertilized three times a week with a commercial fertilizer solution (Jack's Professional 15-5-15 4 Ca 2 Mg fertilizer (manufactured by JR PETERS INC., 6656 Grant Way, Allentown, PA 18106; www.jrpeters.com). Percent visual control assessments were made on a scale of 0 to 100% (where 0 was no control and 100 was complete plant death) at 14 and 21 days after application.

TABLE 6
Treatments Applied in Trial 1

| Trt | | | Mixing Partner | MP rate g ai or |
|---|---|---|---|---|
| No. | Compound No. | Rate g ae/ha | (MP) | ae/ha |
| 1 | B2 | 0 | none | |
| 2 | B2 | 375 | none | |
| 3 | B2 | 560 | none | |
| 4 | B2 | 1120 | none | |
| 5 | B2 | 0 | Saflufenacil | 25 |
| 6 | B2 | 375 | Saflufenacil | 25 |
| 7 | B2 | 560 | Saflufenacil | 25 |
| 8 | B2 | 1120 | Saflufenacil | 25 |
| 9 | B2 | 0 | Saflufenacil | 50 |
| 10 | B2 | 375 | Saflufenacil | 50 |
| 11 | B2 | 560 | Saflufenacil | 50 |
| 12 | B2 | 1120 | Saflufenacil | 50 |
| 13 | B2 | 0 | Saflufenacil | 75 |
| 14 | B2 | 375 | Saflufenacil | 75 |
| 15 | B2 | 560 | Saflufenacil | 75 |
| 16 | B2 | 1120 | Saflufenacil | 75 |
| 17 | B2 | 0 | Sulfentrazone | 140 |
| 18 | B2 | 375 | Sulfentrazone | 140 |
| 19 | B2 | 560 | Sulfentrazone | 140 |
| 20 | B2 | 1120 | Sulfentrazone | 140 |
| 21 | B2 | 0 | Sulfentrazone | 210 |
| 22 | B2 | 375 | Sulfentrazone | 210 |
| 23 | B2 | 560 | Sulfentrazone | 210 |
| 24 | B2 | 1120 | Sulfentrazone | 210 |
| 25 | B2 | 0 | Sulfentrazone | 420 |
| 26 | B2 | 375 | Sulfentrazone | 420 |
| 27 | B2 | 560 | Sulfentrazone | 420 |
| 28 | B2 | 1120 | Sulfentrazone | 420 |
| 29 | B2 | 0 | Glyphosate | 375 |
| 30 | B2 | 375 | Glyphosate | 375 |
| 31 | B2 | 560 | Glyphosate | 375 |
| 32 | B2 | 1120 | Glyphosate | 375 |
| 33 | B2 | 0 | Glyphosate | 1120 |
| 34 | B2 | 375 | Glyphosate | 1120 |
| 35 | B2 | 560 | Glyphosate | 1120 |
| 36 | B2 | 1120 | Glyphosate | 1120 |
| 37 | B2 | 0 | Glyphosate | 1680 |
| 38 | B2 | 375 | Glyphosate | 1680 |
| 39 | B2 | 560 | Glyphosate | 1680 |
| 40 | B2 | 1120 | Glyphosate | 1680 |
| 41 | Untreated | | | |

TABLE 7
Treatments Applied in Trial 2

| | | | Mixing Partner | MP rate g ai |
|---|---|---|---|---|
| Trt No. | Compound No. | Rate g ae/ha | (MP) | or ae/ha |
| 1 | B2 | 0 | none | |
| 2 | B2 | 560 | none | |
| 3 | B2 | 1120 | none | |
| 4 | B2 | 0 | Glufosinate | 200 |
| 5 | B2 | 560 | Glufosinate | 200 |
| 6 | B2 | 1120 | Glufosinate | 200 |
| 7 | B2 | 0 | Glufosinate | 600 |
| 8 | B2 | 560 | Glufosinate | 600 |
| 9 | B2 | 1120 | Glufosinate | 600 |
| 10 | B2 | 0 | Glufosinate | 900 |
| 11 | B2 | 560 | Glufosinate | 900 |
| 12 | B2 | 1120 | Glufosinate | 900 |
| 13 | B2 | 0 | Diclosulam | 25 |
| 14 | B2 | 560 | Diclosulam | 25 |
| 15 | B2 | 1120 | Diclosulam | 25 |
| 16 | B2 | 0 | Diclosulam | 35 |
| 17 | B2 | 560 | Diclosulam | 35 |
| 18 | B2 | 1120 | Diclosulam | 35 |
| 19 | B2 | 0 | Arylex | 5 |
| 20 | B2 | 560 | Arylex | 5 |
| 21 | B2 | 1120 | Arylex | 5 |
| 22 | B2 | 0 | Arylex | 6.5 |
| 23 | B2 | 560 | Arylex | 6.5 |
| 24 | B2 | 1120 | Arylex | 6.5 |
| 25 | B2 | 0 | Mesotrione | 75 |
| 26 | B2 | 560 | Mesotrione | 75 |
| 27 | B2 | 1120 | Mesotrione | 75 |
| 28 | B2 | 0 | Mesotrione | 150 |
| 29 | B2 | 560 | Mesotrione | 150 |
| 30 | B2 | 1120 | Mesotrione | 150 |
| 31 | Untreated | | | |

TABLE 8
Treatments Applied in Bridging Trial 1

| Trt | Treatment | | Mixing Partner | MP rate g ai |
|---|---|---|---|---|
| No. | Name / No. | Rate g ae/ha | (MP) | or ae/ha |
| 1 | Solvent Check / | 0 | 0 | 0 |
| | Untreated | | | |
| 2 | D10 | 560 | none | 0 |
| 3 | D10 | 1120 | none | 0 |
| 4 | D10 | 0 | diclosulam | 35 |
| 5 | D10 | 560 | diclosulam | 35 |
| 6 | D10 | 1120 | diclosulam | 35 |
| 7 | D10 | 0 | Arylex | 5 |
| 8 | D10 | 560 | Arylex | 5 |
| 9 | D10 | 1120 | Arylex | 5 |
| 10 | D10 | 0 | Mesotrione | 150 |
| 11 | D10 | 560 | Mesotrione | 150 |
| 12 | D10 | 1120 | Mesotrione | 150 |
| 13 | D10 | 0 | Glyphosate | 1120 |
| 14 | D10 | 560 | Glyphosate | 1120 |
| 15 | D10 | 1120 | Glyphosate | 1120 |
| 16 | D10 | 0 | Glufosinate | 600 |
| 17 | D10 | 560 | Glufosinate | 600 |
| 18 | D10 | 1120 | Glufosinate | 600 |
| 19 | D8 | 560 | none | 0 |
| 20 | D8 | 1120 | none | 0 |
| 21 | D8 | 560 | diclosulam | 35 |
| 22 | D8 | 1120 | diclosulam | 35 |
| 23 | D8 | 560 | Arylex | 5 |
| 24 | D8 | 1120 | Arylex | 5 |
| 25 | D8 | 560 | Mesotrione | 150 |
| 26 | D8 | 1120 | Mesotrione | 150 |
| 28 | D8 | 1120 | Glyphosate | 1120 |
| 29 | D8 | 560 | Glufosinate | 600 |
| 30 | D8 | 1120 | Glufosinate | 600 |
| 31 | D27 | 560 | none | 0 |
| 32 | D27 | 1120 | none | 0 |
| 33 | D27 | 560 | diclosulam | 35 |
| 34 | D27 | 1120 | diclosulam | 35 |
| 35 | D27 | 560 | Arylex | 5 |
| 36 | D27 | 1120 | Arylex | 5 |
| 37 | D27 | 560 | Mesotrione | 150 |
| 38 | D27 | 1120 | Mesotrione | 150 |
| 39 | D27 | 560 | Glyphosate | 1120 |
| 40 | D27 | 1120 | Glyphosate | 1120 |
| 41 | D27 | 560 | Glufosinate | 600 |
| 42 | D27 | 1120 | Glufosinate | 600 |
| 43 | B2 | 1120 | none | 0 |
| 44 | B2 | 1120 | diclosulam | 35 |
| 45 | B2 | 1120 | Arylex | 5 |
| 46 | B2 | 1120 | Mesotrione | 150 |
| 47 | B2 | 1120 | Glyphosate | 1120 |
| 48 | B2 | 1120 | Glufosinate | 600 |

TABLE 9
Treatments Applied in Bridging Trial 2

| Trt | | Rate g | Mixing Partner | MP rate g ai |
|---|---|---|---|---|
| No. | Compound No. | ae/ha | (MP) | or ae/ha |
| 1 | Solvent | 0 | 0 | 0 |
| | Check/Untreated | | | |
| 2 | B2 | 375 | none | |
| 3 | B2 | 560 | none | |
| 4 | B2 | 1120 | none | |
| 5 | B2 | 0 | Trifludimoxazin | 12.5 |
| 6 | B2 | 375 | Trifludimoxazin | 12.5 |
| 7 | B2 | 560 | Trifludimoxazin | 12.5 |
| 8 | B2 | 1120 | Trifludimoxazin | 12.5 |
| 9 | B2 | 0 | Trifludimoxazin | 25 |
| 10 | B2 | 375 | Trifludimoxazin | 25 |
| 11 | B2 | 560 | Trifludimoxazin | 25 |
| 12 | B2 | 1120 | Trifludimoxazin | 25 |
| 13 | B2 | 0 | Trifludimoxazin | 50 |
| 14 | B2 | 375 | Trifludimoxazin | 50 |
| 15 | B2 | 560 | Trifludimoxazin | 50 |
| 16 | B2 | 1120 | Trifludimoxazin | 50 |
| 17 | D10 | 560 | none | 0 |
| 18 | D10 | 1120 | none | 0 |
| 19 | D10 | 560 | Trifludimoxazin | 12.5 |
| 20 | D10 | 1120 | Trifludimoxazin | 12.5 |
| 21 | D10 | 560 | Trifludimoxazin | 50 |
| 22 | D10 | 1120 | Trifludimoxazin | 50 |
| 23 | D10 | 560 | Saflufenacil | 25 |
| 24 | D10 | 1120 | Saflufenacil | 25 |
| 25 | D10 | 560 | Saflufenacil | 75 |
| 26 | D10 | 1120 | Saflufenacil | 75 |
| 27 | D8 | 560 | none | 0 |
| 28 | D8 | 1120 | none | 0 |
| 29 | D8 | 560 | Trifludimoxazin | 12.5 |
| 30 | D8 | 1120 | Trifludimoxazin | 12.5 |
| 31 | D8 | 560 | Trifludimoxazin | 50 |
| 32 | D8 | 1120 | Trifludimoxazin | 50 |
| 33 | D8 | 560 | Saflufenacil | 25 |
| 34 | D8 | 1120 | Saflufenacil | 25 |
| 35 | D8 | 560 | Saflufenacil | 75 |
| 36 | D8 | 1120 | Saflufenacil | 75 |
| 37 | D27 | 560 | none | 0 |
| 38 | D27 | 1120 | none | 0 |
| 39 | D27 | 560 | Trifludimoxazin | 12.5 |
| 40 | D27 | 1120 | Trifludimoxazin | 12.5 |
| 41 | D27 | 560 | Trifludimoxazin | 50 |
| 42 | D27 | 1120 | Trifludimoxazin | 50 |
| 43 | D27 | 560 | Saflufenacil | 25 |
| 44 | D27 | 1120 | Saflufenacil | 25 |
| 45 | D27 | 560 | Saflufenacil | 75 |
| 46 | D27 | 1120 | Saflufenacil | 75 |

TABLE 10
Treatments Applied in Bridging Trial 3

| | | | Mixing Partner | MP rate g ai or |
|---|---|---|---|---|
| Trt No. | Comp No. | Rate g ae/ha | (MP) | ae/ha |
| 1 | Solvent Check | 0 | 0 | 0 |
| 2 | B2 | 375 | none | |
| 3 | B2 | 560 | none | |
| 4 | B2 | 1120 | none | |
| 5 | B2 | 0 | Sulfentrazone | 140 |
| 6 | B2 | 375 | Sulfentrazone | 140 |
| 7 | B2 | 560 | Sulfentrazone | 140 |
| 8 | B2 | 1120 | Sulfentrazone | 140 |
| 9 | B2 | 0 | Sulfentrazone | 210 |
| 10 | B2 | 375 | Sulfentrazone | 210 |
| 11 | B2 | 560 | Sulfentrazone | 210 |
| 12 | B2 | 1120 | Sulfentrazone | 210 |
| 13 | B2 | 0 | Sulfentrazone | 420 |
| 14 | B2 | 375 | Sulfentrazone | 420 |
| 15 | B2 | 560 | Sulfentrazone | 420 |
| 16 | B2 | 1120 | Sulfentrazone | 420 |
| 17 | D10 | 560 | none | 0 |
| 18 | D10 | 1120 | none | 0 |
| 19 | D10 | 560 | Sulfentrazone | 130 |
| 20 | D10 | 1120 | Sulfentrazone | 130 |
| 21 | D10 | 560 | Sulfentrazone | 347 |
| 22 | D10 | 1120 | Sulfentrazone | 347 |
| 23 | D8 | 560 | none | 0 |
| 24 | D8 | 1120 | none | 0 |
| 25 | D8 | 560 | Sulfentrazone | 130 |
| 26 | D8 | 1120 | Sulfentrazone | 130 |
| 27 | D8 | 560 | Sulfentrazone | 347 |
| 28 | D8 | 1120 | Sulfentrazone | 347 |
| 29 | D27 | 560 | none | 0 |
| 30 | D27 | 1120 | none | 0 |
| 31 | D27 | 560 | Sulfentrazone | 130 |
| 32 | D27 | 1120 | Sulfentrazone | 130 |
| 33 | D27 | 560 | Sulfentrazone | 347 |
| 34 | D27 | 1120 | Sulfentrazone | 347 |
| 35 | Untreated | | | |

Mixture Trials Results

| Trial | Trt | Compound | Rate g | Mixing Partner | MP rate g | | | % |
|---|---|---|---|---|---|---|---|---|
| No. | No. | No. | ae/ha | (MP) | ai or ae/ha | Species | DAA | Control |
| 1 | 1 | Solv. Ck | 0 | none | 0 | ABUTH | 21 | 0 |
| | | untreated | | | | | | |
| 1 | 1 | Solv. Ck | 0 | none | 0 | ABUTH | 21 | 0 |
| | | untreated | | | | | | |
| 1 | 1 | Solv. Ck | 0 | none | 0 | ABUTH | 21 | 0 |
| | | untreated | | | | | | |
| 1 | 2 | B2 | 375 | none | 0 | ABUTH | 21 | 65 |
| 1 | 2 | B2 | 375 | none | 0 | ABUTH | 21 | 75 |
| 1 | 2 | B2 | 375 | none | 0 | ABUTH | 21 | 60 |
| 1 | 3 | B2 | 560 | none | 0 | ABUTH | 21 | 80 |
| 1 | 3 | B2 | 560 | none | 0 | ABUTH | 21 | 60 |
| 1 | 3 | B2 | 560 | none | 0 | ABUTH | 21 | 75 |
| 1 | 4 | B2 | 1120 | none | 0 | ABUTH | 21 | 90 |
| 1 | 4 | B2 | 1120 | none | 0 | ABUTH | 21 | 85 |
| 1 | 4 | B2 | 1120 | none | 0 | ABUTH | 21 | 90 |
| 1 | 5 | B2 | 0 | Saflufenacil | 25 | ABUTH | 21 | 100 |
| 1 | 5 | B2 | 0 | Saflufenacil | 25 | ABUTH | 21 | 100 |
| 1 | 5 | B2 | 0 | Saflufenacil | 25 | ABUTH | 21 | 100 |
| 1 | 6 | B2 | 375 | Saflufenacil | 25 | ABUTH | 21 | 100 |
| 1 | 6 | B2 | 375 | Saflufenacil | 25 | ABUTH | 21 | 100 |
| 1 | 6 | B2 | 375 | Saflufenacil | 25 | ABUTH | 21 | 100 |
| 1 | 7 | B2 | 560 | Saflufenacil | 25 | ABUTH | 21 | 100 |
| 1 | 7 | B2 | 560 | Saflufenacil | 25 | ABUTH | 21 | 100 |
| 1 | 7 | B2 | 560 | Saflufenacil | 25 | ABUTH | 21 | 100 |
| 1 | 8 | B2 | 1120 | Saflufenacil | 25 | ABUTH | 21 | 100 |
| 1 | 8 | B2 | 1120 | Saflufenacil | 25 | ABUTH | 21 | 100 |
| 1 | 8 | B2 | 1120 | Saflufenacil | 25 | ABUTH | 21 | 100 |
| 1 | 9 | B2 | 0 | Saflufenacil | 50 | ABUTH | 21 | 100 |
| 1 | 9 | B2 | 0 | Saflufenacil | 50 | ABUTH | 21 | 100 |
| 1 | 9 | B2 | 0 | Saflufenacil | 50 | ABUTH | 21 | 100 |
| 1 | 10 | B2 | 375 | Saflufenacil | 50 | ABUTH | 21 | 100 |
| 1 | 10 | B2 | 375 | Saflufenacil | 50 | ABUTH | 21 | 100 |
| 1 | 10 | B2 | 375 | Saflufenacil | 50 | ABUTH | 21 | 100 |
| 1 | 11 | B2 | 560 | Saflufenacil | 50 | ABUTH | 21 | 100 |
| 1 | 11 | B2 | 560 | Saflufenacil | 50 | ABUTH | 21 | 100 |
| 1 | 11 | B2 | 560 | Saflufenacil | 50 | ABUTH | 21 | 100 |
| 1 | 12 | B2 | 1120 | Saflufenacil | 50 | ABUTH | 21 | 100 |
| 1 | 12 | B2 | 1120 | Saflufenacil | 50 | ABUTH | 21 | 100 |
| 1 | 12 | B2 | 1120 | Saflufenacil | 50 | ABUTH | 21 | 100 |
| 1 | 13 | B2 | 0 | Saflufenacil | 75 | ABUTH | 21 | 100 |
| 1 | 13 | B2 | 0 | Saflufenacil | 75 | ABUTH | 21 | 100 |
| 1 | 13 | B2 | 0 | Saflufenacil | 75 | ABUTH | 21 | 100 |
| 1 | 14 | B2 | 375 | Saflufenacil | 75 | ABUTH | 21 | 100 |
| 1 | 14 | B2 | 375 | Saflufenacil | 75 | ABUTH | 21 | 100 |
| 1 | 14 | B2 | 375 | Saflufenacil | 75 | ABUTH | 21 | 100 |
| 1 | 15 | B2 | 560 | Saflufenacil | 75 | ABUTH | 21 | 100 |
| 1 | 15 | B2 | 560 | Saflufenacil | 75 | ABUTH | 21 | 100 |
| 1 | 15 | B2 | 560 | Saflufenacil | 75 | ABUTH | 21 | 100 |
| 1 | 16 | B2 | 1120 | Saflufenacil | 75 | ABUTH | 21 | 100 |
| 1 | 16 | B2 | 1120 | Saflufenacil | 75 | ABUTH | 21 | 100 |
| 1 | 16 | B2 | 1120 | Saflufenacil | 75 | ABUTH | 21 | 100 |
| 1 | 17 | B2 | 0 | Sulfentrazone | 140 | ABUTH | 21 | 45 |
| 1 | 17 | B2 | 0 | Sulfentrazone | 140 | ABUTH | 21 | 78 |
| 1 | 17 | B2 | 0 | Sulfentrazone | 140 | ABUTH | 21 | 80 |
| 1 | 18 | B2 | 375 | Sulfentrazone | 140 | ABUTH | 21 | 80 |
| 1 | 18 | B2 | 375 | Sulfentrazone | 140 | ABUTH | 21 | 50 |
| 1 | 18 | B2 | 375 | Sulfentrazone | 140 | ABUTH | 21 | 65 |
| 1 | 19 | B2 | 560 | Sulfentrazone | 140 | ABUTH | 21 | 65 |
| 1 | 19 | B2 | 560 | Sulfentrazone | 140 | ABUTH | 21 | 80 |
| 1 | 19 | B2 | 560 | Sulfentrazone | 140 | ABUTH | 21 | 100 |
| 1 | 20 | B2 | 1120 | Sulfentrazone | 140 | ABUTH | 21 | 98 |
| 1 | 20 | B2 | 1120 | Sulfentrazone | 140 | ABUTH | 21 | 99 |
| 1 | 20 | B2 | 1120 | Sulfentrazone | 140 | ABUTH | 21 | 85 |
| 1 | 21 | B2 | 0 | Sulfentrazone | 210 | ABUTH | 21 | 65 |
| 1 | 21 | B2 | 0 | Sulfentrazone | 210 | ABUTH | 21 | 80 |
| 1 | 21 | B2 | 0 | Sulfentrazone | 210 | ABUTH | 21 | 99 |
| 1 | 22 | B2 | 375 | Sulfentrazone | 210 | ABUTH | 21 | 98 |
| 1 | 22 | B2 | 375 | Sulfentrazone | 210 | ABUTH | 21 | 99 |
| 1 | 22 | B2 | 375 | Sulfentrazone | 210 | ABUTH | 21 | 98 |
| 1 | 23 | B2 | 560 | Sulfentrazone | 210 | ABUTH | 21 | 99 |
| 1 | 23 | B2 | 560 | Sulfentrazone | 210 | ABUTH | 21 | 100 |
| 1 | 23 | B2 | 560 | Sulfentrazone | 210 | ABUTH | 21 | 99 |
| 1 | 24 | B2 | 1120 | Sulfentrazone | 210 | ABUTH | 21 | 99 |
| 1 | 24 | B2 | 1120 | Sulfentrazone | 210 | ABUTH | 21 | 99 |
| 1 | 24 | B2 | 1120 | Sulfentrazone | 210 | ABUTH | 21 | 100 |
| 1 | 25 | B2 | 0 | Sulfentrazone | 420 | ABUTH | 21 | 80 |
| 1 | 25 | B2 | 0 | Sulfentrazone | 420 | ABUTH | 21 | 99 |
| 1 | 25 | B2 | 0 | Sulfentrazone | 420 | ABUTH | 21 | 100 |
| 1 | 26 | B2 | 375 | Sulfentrazone | 420 | ABUTH | 21 | 100 |
| 1 | 26 | B2 | 375 | Sulfentrazone | 420 | ABUTH | 21 | 100 |
| 1 | 26 | B2 | 375 | Sulfentrazone | 420 | ABUTH | 21 | 100 |
| 1 | 27 | B2 | 560 | Sulfentrazone | 420 | ABUTH | 21 | 99 |
| 1 | 27 | B2 | 560 | Sulfentrazone | 420 | ABUTH | 21 | 100 |
| 1 | 27 | B2 | 560 | Sulfentrazone | 420 | ABUTH | 21 | 100 |
| 1 | 28 | B2 | 1120 | Sulfentrazone | 420 | ABUTH | 21 | 99 |
| 1 | 28 | B2 | 1120 | Sulfentrazone | 420 | ABUTH | 21 | 100 |
| 1 | 28 | B2 | 1120 | Sulfentrazone | 420 | ABUTH | 21 | 100 |
| 1 | 29 | B2 | 0 | Glyphosate | 375 | ABUTH | 21 | 85 |
| 1 | 29 | B2 | 0 | Glyphosate | 375 | ABUTH | 21 | 85 |
| 1 | 29 | B2 | 0 | Glyphosate | 375 | ABUTH | 21 | 90 |
| 1 | 30 | B2 | 375 | Glyphosate | 375 | ABUTH | 21 | 97 |
| 1 | 30 | B2 | 375 | Glyphosate | 375 | ABUTH | 21 | 100 |
| 1 | 30 | B2 | 375 | Glyphosate | 375 | ABUTH | 21 | 100 |
| 1 | 31 | B2 | 560 | Glyphosate | 375 | ABUTH | 21 | 98 |
| 1 | 31 | B2 | 560 | Glyphosate | 375 | ABUTH | 21 | 95 |
| 1 | 31 | B2 | 560 | Glyphosate | 375 | ABUTH | 21 | 98 |
| 1 | 32 | B2 | 1120 | Glyphosate | 375 | ABUTH | 21 | 98 |
| 1 | 32 | B2 | 1120 | Glyphosate | 375 | ABUTH | 21 | 97 |
| 1 | 32 | B2 | 1120 | Glyphosate | 375 | ABUTH | 21 | 97 |
| 1 | 33 | B2 | 0 | Glyphosate | 1120 | ABUTH | 21 | 100 |
| 1 | 33 | B2 | 0 | Glyphosate | 1120 | ABUTH | 21 | 100 |
| 1 | 33 | B2 | 0 | Glyphosate | 1120 | ABUTH | 21 | 100 |
| 1 | 34 | B2 | 375 | Glyphosate | 1120 | ABUTH | 21 | 100 |
| 1 | 34 | B2 | 375 | Glyphosate | 1120 | ABUTH | 21 | 100 |
| 1 | 34 | B2 | 375 | Glyphosate | 1120 | ABUTH | 21 | 100 |
| 1 | 35 | B2 | 560 | Glyphosate | 1120 | ABUTH | 21 | 100 |
| 1 | 35 | B2 | 560 | Glyphosate | 1120 | ABUTH | 21 | 100 |
| 1 | 35 | B2 | 560 | Glyphosate | 1120 | ABUTH | 21 | 100 |
| 1 | 36 | B2 | 1120 | Glyphosate | 1120 | ABUTH | 21 | 100 |
| 1 | 36 | B2 | 1120 | Glyphosate | 1120 | ABUTH | 21 | 100 |
| 1 | 36 | B2 | 1120 | Glyphosate | 1120 | ABUTH | 21 | 100 |
| 1 | 37 | B2 | 0 | Glyphosate | 1680 | ABUTH | 21 | 100 |
| 1 | 37 | B2 | 0 | Glyphosate | 1680 | ABUTH | 21 | 100 |
| 1 | 37 | B2 | 0 | Glyphosate | 1680 | ABUTH | 21 | 100 |
| 1 | 38 | B2 | 375 | Glyphosate | 1680 | ABUTH | 21 | 100 |
| 1 | 38 | B2 | 375 | Glyphosate | 1680 | ABUTH | 21 | 100 |
| 1 | 38 | B2 | 375 | Glyphosate | 1680 | ABUTH | 21 | 100 |
| 1 | 39 | B2 | 560 | Glyphosate | 1680 | ABUTH | 21 | 100 |
| 1 | 39 | B2 | 560 | Glyphosate | 1680 | ABUTH | 21 | 100 |
| 1 | 39 | B2 | 560 | Glyphosate | 1680 | ABUTH | 21 | 100 |
| 1 | 40 | B2 | 1120 | Glyphosate | 1680 | ABUTH | 21 | 100 |
| 1 | 40 | B2 | 1120 | Glyphosate | 1680 | ABUTH | 21 | 100 |
| 1 | 40 | B2 | 1120 | Glyphosate | 1680 | ABUTH | 21 | 100 |
| 1 | 41 | Untreated | | | | ABUTH | 21 | 0 |
| 1 | 41 | Untreated | | | | ABUTH | 21 | 0 |
| 1 | 41 | Untreated | | | | ABUTH | 21 | 0 |
| 1 | 1 | Solv. Ck | 0 | none | 0 | AMATA | 21 | 0 |
| | | untreated | | | | | | |
| 1 | 1 | Solv. Ck | 0 | none | 0 | AMATA | 21 | 0 |
| | | untreated | | | | | | |
| 1 | 1 | Solv. Ck | 0 | none | 0 | AMATA | 21 | 0 |
| | | untreated | | | | | | |
| 1 | 2 | B2 | 375 | none | 0 | AMATA | 21 | 70 |
| 1 | 2 | B2 | 375 | none | 0 | AMATA | 21 | 85 |
| 1 | 2 | B2 | 375 | none | 0 | AMATA | 21 | 75 |
| 1 | 3 | B2 | 560 | none | 0 | AMATA | 21 | 65 |
| 1 | 3 | B2 | 560 | none | 0 | AMATA | 21 | 75 |
| 1 | 3 | B2 | 560 | none | 0 | AMATA | 21 | 75 |
| 1 | 4 | B2 | 1120 | none | 0 | AMATA | 21 | 100 |
| 1 | 4 | B2 | 1120 | none | 0 | AMATA | 21 | 100 |
| 1 | 4 | B2 | 1120 | none | 0 | AMATA | 21 | 100 |
| 1 | 5 | B2 | 0 | Saflufenacil | 25 | AMATA | 21 | 85 |
| 1 | 5 | B2 | 0 | Saflufenacil | 25 | AMATA | 21 | 100 |
| 1 | 5 | B2 | 0 | Saflufenacil | 25 | AMATA | 21 | 100 |
| 1 | 6 | B2 | 375 | Saflufenacil | 25 | AMATA | 21 | 100 |
| 1 | 6 | B2 | 375 | Saflufenacil | 25 | AMATA | 21 | 100 |
| 1 | 6 | B2 | 375 | Saflufenacil | 25 | AMATA | 21 | 100 |
| 1 | 7 | B2 | 560 | Saflufenacil | 25 | AMATA | 21 | 100 |
| 1 | 7 | B2 | 560 | Saflufenacil | 25 | AMATA | 21 | 100 |
| 1 | 7 | B2 | 560 | Saflufenacil | 25 | AMATA | 21 | 100 |
| 1 | 8 | B2 | 1120 | Saflufenacil | 25 | AMATA | 21 | 100 |
| 1 | 8 | B2 | 1120 | Saflufenacil | 25 | AMATA | 21 | 100 |
| 1 | 8 | B2 | 1120 | Saflufenacil | 25 | AMATA | 21 | 100 |
| 1 | 9 | B2 | 0 | Saflufenacil | 50 | AMATA | 21 | 100 |
| 1 | 9 | B2 | 0 | Saflufenacil | 50 | AMATA | 21 | 100 |
| 1 | 9 | B2 | 0 | Saflufenacil | 50 | AMATA | 21 | 100 |
| 1 | 10 | B2 | 375 | Saflufenacil | 50 | AMATA | 21 | 100 |
| 1 | 10 | B2 | 375 | Saflufenacil | 50 | AMATA | 21 | 100 |
| 1 | 10 | B2 | 375 | Saflufenacil | 50 | AMATA | 21 | 100 |
| 1 | 11 | B2 | 560 | Saflufenacil | 50 | AMATA | 21 | 100 |
| 1 | 11 | B2 | 560 | Saflufenacil | 50 | AMATA | 21 | 100 |
| 1 | 11 | B2 | 560 | Saflufenacil | 50 | AMATA | 21 | 100 |
| 1 | 12 | B2 | 1120 | Saflufenacil | 50 | AMATA | 21 | 100 |
| 1 | 12 | B2 | 1120 | Saflufenacil | 50 | AMATA | 21 | 100 |
| 1 | 12 | B2 | 1120 | Saflufenacil | 50 | AMATA | 21 | 100 |
| 1 | 13 | B2 | 0 | Saflufenacil | 75 | AMATA | 21 | 100 |
| 1 | 13 | B2 | 0 | Saflufenacil | 75 | AMATA | 21 | 100 |
| 1 | 13 | B2 | 0 | Saflufenacil | 75 | AMATA | 21 | 100 |
| 1 | 14 | B2 | 375 | Saflufenacil | 75 | AMATA | 21 | 100 |
| 1 | 14 | B2 | 375 | Saflufenacil | 75 | AMATA | 21 | 100 |
| 1 | 14 | B2 | 375 | Saflufenacil | 75 | AMATA | 21 | 100 |
| 1 | 15 | B2 | 560 | Saflufenacil | 75 | AMATA | 21 | 100 |
| 1 | 15 | B2 | 560 | Saflufenacil | 75 | AMATA | 21 | 100 |
| 1 | 15 | B2 | 560 | Saflufenacil | 75 | AMATA | 21 | 100 |
| 1 | 16 | B2 | 1120 | Saflufenacil | 75 | AMATA | 21 | 100 |
| 1 | 16 | B2 | 1120 | Saflufenacil | 75 | AMATA | 21 | 100 |
| 1 | 16 | B2 | 1120 | Saflufenacil | 75 | AMATA | 21 | 100 |
| 1 | 17 | B2 | 0 | Sulfentrazone | 140 | AMATA | 21 | 98 |
| 1 | 17 | B2 | 0 | Sulfentrazone | 140 | AMATA | 21 | 90 |
| 1 | 17 | B2 | 0 | Sulfentrazone | 140 | AMATA | 21 | 100 |
| 1 | 18 | B2 | 375 | Sulfentrazone | 140 | AMATA | 21 | 95 |
| 1 | 18 | B2 | 375 | Sulfentrazone | 140 | AMATA | 21 | 100 |
| 1 | 18 | B2 | 375 | Sulfentrazone | 140 | AMATA | 21 | 100 |
| 1 | 19 | B2 | 560 | Sulfentrazone | 140 | AMATA | 21 | 100 |
| 1 | 19 | B2 | 560 | Sulfentrazone | 140 | AMATA | 21 | 100 |
| 1 | 19 | B2 | 560 | Sulfentrazone | 140 | AMATA | 21 | 100 |
| 1 | 20 | B2 | 1120 | Sulfentrazone | 140 | AMATA | 21 | 100 |
| 1 | 20 | B2 | 1120 | Sulfentrazone | 140 | AMATA | 21 | 100 |
| 1 | 20 | B2 | 1120 | Sulfentrazone | 140 | AMATA | 21 | 100 |
| 1 | 21 | B2 | 0 | Sulfentrazone | 210 | AMATA | 21 | 98 |
| 1 | 21 | B2 | 0 | Sulfentrazone | 210 | AMATA | 21 | 99 |
| 1 | 21 | B2 | 0 | Sulfentrazone | 210 | AMATA | 21 | 95 |
| 1 | 22 | B2 | 375 | Sulfentrazone | 210 | AMATA | 21 | 100 |
| 1 | 22 | B2 | 375 | Sulfentrazone | 210 | AMATA | 21 | 100 |
| 1 | 22 | B2 | 375 | Sulfentrazone | 210 | AMATA | 21 | 100 |
| 1 | 23 | B2 | 560 | Sulfentrazone | 210 | AMATA | 21 | 100 |
| 1 | 23 | B2 | 560 | Sulfentrazone | 210 | AMATA | 21 | 100 |
| 1 | 23 | B2 | 560 | Sulfentrazone | 210 | AMATA | 21 | 100 |
| 1 | 24 | B2 | 1120 | Sulfentrazone | 210 | AMATA | 21 | 100 |
| 1 | 24 | B2 | 1120 | Sulfentrazone | 210 | AMATA | 21 | 100 |
| 1 | 24 | B2 | 1120 | Sulfentrazone | 210 | AMATA | 21 | 100 |
| 1 | 25 | B2 | 0 | Sulfentrazone | 420 | AMATA | 21 | 95 |
| 1 | 25 | B2 | 0 | Sulfentrazone | 420 | AMATA | 21 | 100 |
| 1 | 25 | B2 | 0 | Sulfentrazone | 420 | AMATA | 21 | 100 |
| 1 | 26 | B2 | 375 | Sulfentrazone | 420 | AMATA | 21 | 100 |
| 1 | 26 | B2 | 375 | Sulfentrazone | 420 | AMATA | 21 | 100 |
| 1 | 26 | B2 | 375 | Sulfentrazone | 420 | AMATA | 21 | 100 |
| 1 | 27 | B2 | 560 | Sulfentrazone | 420 | AMATA | 21 | 78 |
| 1 | 27 | B2 | 560 | Sulfentrazone | 420 | AMATA | 21 | 100 |
| 1 | 27 | B2 | 560 | Sulfentrazone | 420 | AMATA | 21 | 100 |
| 1 | 28 | B2 | 1120 | Sulfentrazone | 420 | AMATA | 21 | 100 |
| 1 | 28 | B2 | 1120 | Sulfentrazone | 420 | AMATA | 21 | 100 |
| 1 | 28 | B2 | 1120 | Sulfentrazone | 420 | AMATA | 21 | 100 |
| 1 | 29 | B2 | 0 | Glyphosate | 375 | AMATA | 21 | 95 |
| 1 | 29 | B2 | 0 | Glyphosate | 375 | AMATA | 21 | 95 |
| 1 | 29 | B2 | 0 | Glyphosate | 375 | AMATA | 21 | 100 |
| 1 | 30 | B2 | 375 | Glyphosate | 375 | AMATA | 21 | 100 |
| 1 | 30 | B2 | 375 | Glyphosate | 375 | AMATA | 21 | 100 |
| 1 | 30 | B2 | 375 | Glyphosate | 375 | AMATA | 21 | 100 |
| 1 | 31 | B2 | 560 | Glyphosate | 375 | AMATA | 21 | 100 |
| 1 | 31 | B2 | 560 | Glyphosate | 375 | AMATA | 21 | 100 |
| 1 | 31 | B2 | 560 | Glyphosate | 375 | AMATA | 21 | 100 |
| 1 | 32 | B2 | 1120 | Glyphosate | 375 | AMATA | 21 | 100 |
| 1 | 32 | B2 | 1120 | Glyphosate | 375 | AMATA | 21 | 100 |
| 1 | 32 | B2 | 1120 | Glyphosate | 375 | AMATA | 21 | 100 |
| 1 | 33 | B2 | 0 | Glyphosate | 1120 | AMATA | 21 | 100 |
| 1 | 33 | B2 | 0 | Glyphosate | 1120 | AMATA | 21 | 100 |
| 1 | 33 | B2 | 0 | Glyphosate | 1120 | AMATA | 21 | 100 |
| 1 | 34 | B2 | 375 | Glyphosate | 1120 | AMATA | 21 | 100 |
| 1 | 34 | B2 | 375 | Glyphosate | 1120 | AMATA | 21 | 100 |
| 1 | 34 | B2 | 375 | Glyphosate | 1120 | AMATA | 21 | 100 |
| 1 | 35 | B2 | 560 | Glyphosate | 1120 | AMATA | 21 | 100 |
| 1 | 35 | B2 | 560 | Glyphosate | 1120 | AMATA | 21 | 100 |
| 1 | 35 | B2 | 560 | Glyphosate | 1120 | AMATA | 21 | 100 |
| 1 | 36 | B2 | 1120 | Glyphosate | 1120 | AMATA | 21 | 100 |
| 1 | 36 | B2 | 1120 | Glyphosate | 1120 | AMATA | 21 | 100 |
| 1 | 36 | B2 | 1120 | Glyphosate | 1120 | AMATA | 21 | 100 |
| 1 | 37 | B2 | 0 | Glyphosate | 1680 | AMATA | 21 | 100 |
| 1 | 37 | B2 | 0 | Glyphosate | 1680 | AMATA | 21 | 100 |
| 1 | 37 | B2 | 0 | Glyphosate | 1680 | AMATA | 21 | 99 |
| 1 | 38 | B2 | 375 | Glyphosate | 1680 | AMATA | 21 | 100 |
| 1 | 38 | B2 | 375 | Glyphosate | 1680 | AMATA | 21 | 100 |
| 1 | 38 | B2 | 375 | Glyphosate | 1680 | AMATA | 21 | 100 |
| 1 | 39 | B2 | 560 | Glyphosate | 1680 | AMATA | 21 | 100 |
| 1 | 39 | B2 | 560 | Glyphosate | 1680 | AMATA | 21 | 100 |
| 1 | 39 | B2 | 560 | Glyphosate | 1680 | AMATA | 21 | 100 |
| 1 | 40 | B2 | 1120 | Glyphosate | 1680 | AMATA | 21 | 100 |
| 1 | 40 | B2 | 1120 | Glyphosate | 1680 | AMATA | 21 | 100 |
| 1 | 40 | B2 | 1120 | Glyphosate | 1680 | AMATA | 21 | 100 |
| 1 | 41 | Untreated | | | | AMATA | 21 | 0 |
| 1 | 41 | Untreated | | | | AMATA | 21 | 0 |
| 1 | 41 | Untreated | | | | AMATA | 21 | 0 |
| 1 | 1 | Solv. Ck | 0 | none | 0 | CHEAL | 21 | |
| | | untreated | | | | | | |
| 1 | 1 | Solv. Ck | 0 | none | 0 | CHEAL | 21 | 0 |
| | | untreated | | | | | | |
| 1 | | Solv. Ck | 0 | none | 0 | CHEAL | 21 | |
| | | untreated | | | | | | |
| 1 | 2 | B2 | 375 | none | 0 | CHEAL | 21 | 75 |
| 1 | 2 | B2 | 375 | none | 0 | CHEAL | 21 | 80 |
| 1 | 2 | B2 | 375 | none | 0 | CHEAL | 21 | 75 |
| 1 | 3 | B2 | 560 | none | 0 | CHEAL | 21 | 80 |
| 1 | 3 | B2 | 560 | none | 0 | CHEAL | 21 | 95 |
| 1 | 3 | B2 | 560 | none | 0 | CHEAL | 21 | 80 |
| 1 | 4 | B2 | 1120 | none | 0 | CHEAL | 21 | 85 |
| 1 | 4 | B2 | 1120 | none | 0 | CHEAL | 21 | 85 |
| 1 | 4 | B2 | 1120 | none | 0 | CHEAL | 21 | 80 |
| 1 | 5 | B2 | 0 | Saflufenacil | 25 | CHEAL | 21 | 95 |
| 1 | 5 | B2 | 0 | Saflufenacil | 25 | CHEAL | 21 | 100 |
| 1 | 5 | B2 | 0 | Saflufenacil | 25 | CHEAL | 21 | 75 |
| 1 | 6 | B2 | 375 | Saflufenacil | 25 | CHEAL | 21 | 99 |
| 1 | 6 | B2 | 375 | Saflufenacil | 25 | CHEAL | 21 | 99 |
| 1 | 6 | B2 | 375 | Saflufenacil | 25 | CHEAL | 21 | 99 |
| 1 | 7 | B2 | 560 | Saflufenacil | 25 | CHEAL | 21 | 100 |
| 1 | 7 | B2 | 560 | Saflufenacil | 25 | CHEAL | 21 | 100 |
| 1 | 7 | B2 | 560 | Saflufenacil | 25 | CHEAL | 21 | 100 |
| 1 | 8 | B2 | 1120 | Saflufenacil | 25 | CHEAL | 21 | 99 |
| 1 | 8 | B2 | 1120 | Saflufenacil | 25 | CHEAL | 21 | 100 |
| 1 | 8 | B2 | 1120 | Saflufenacil | 25 | CHEAL | 21 | 100 |
| 1 | 9 | B2 | 0 | Saflufenacil | 50 | CHEAL | 21 | 95 |
| 1 | 9 | B2 | 0 | Saflufenacil | 50 | CHEAL | 21 | 98 |
| 1 | 9 | B2 | 0 | Saflufenacil | 50 | CHEAL | 21 | 100 |
| 1 | 10 | B2 | 375 | Saflufenacil | 50 | CHEAL | 21 | 100 |
| 1 | 10 | B2 | 375 | Saflufenacil | 50 | CHEAL | 21 | 100 |
| 1 | 10 | B2 | 375 | Saflufenacil | 50 | CHEAL | 21 | 100 |
| 1 | 11 | B2 | 560 | Saflufenacil | 50 | CHEAL | 21 | 100 |
| 1 | 11 | B2 | 560 | Saflufenacil | 50 | CHEAL | 21 | 100 |
| 1 | 11 | B2 | 560 | Saflufenacil | 50 | CHEAL | 21 | 100 |
| 1 | 12 | B2 | 1120 | Saflufenacil | 50 | CHEAL | 21 | 100 |
| 1 | 12 | B2 | 1120 | Saflufenacil | 50 | CHEAL | 21 | 100 |
| 1 | 12 | B2 | 1120 | Saflufenacil | 50 | CHEAL | 21 | 100 |
| 1 | 13 | B2 | 0 | Saflufenacil | 75 | CHEAL | 21 | 98 |
| 1 | 13 | B2 | 0 | Saflufenacil | 75 | CHEAL | 21 | 95 |
| 1 | 13 | B2 | 0 | Saflufenacil | 75 | CHEAL | 21 | 100 |
| 1 | 14 | B2 | 375 | Saflufenacil | 75 | CHEAL | 21 | 100 |
| 1 | 14 | B2 | 375 | Saflufenacil | 75 | CHEAL | 21 | 100 |
| 1 | 14 | B2 | 375 | Saflufenacil | 75 | CHEAL | 21 | 100 |
| 1 | 15 | B2 | 560 | Saflufenacil | 75 | CHEAL | 21 | 100 |
| 1 | 15 | B2 | 560 | Saflufenacil | 75 | CHEAL | 21 | 100 |
| 1 | 15 | B2 | 560 | Saflufenacil | 75 | CHEAL | 21 | 100 |
| 1 | 16 | B2 | 1120 | Saflufenacil | 75 | CHEAL | 21 | 100 |
| 1 | 16 | B2 | 1120 | Saflufenacil | 75 | CHEAL | 21 | 100 |
| 1 | 16 | B2 | 1120 | Saflufenacil | 75 | CHEAL | 21 | 100 |
| 1 | 17 | B2 | 0 | Sulfentrazone | 140 | CHEAL | 21 | 30 |
| 1 | 17 | B2 | 0 | Sulfentrazone | 140 | CHEAL | 21 | 40 |
| 1 | 17 | B2 | 0 | Sulfentrazone | 140 | CHEAL | 21 | 80 |
| 1 | 18 | B2 | 375 | Sulfentrazone | 140 | CHEAL | 21 | 97 |
| 1 | 18 | B2 | 375 | Sulfentrazone | 140 | CHEAL | 21 | 98 |
| 1 | 18 | B2 | 375 | Sulfentrazone | 140 | CHEAL | 21 | 100 |
| 1 | 19 | B2 | 560 | Sulfentrazone | 140 | CHEAL | 21 | 99 |
| 1 | 19 | B2 | 560 | Sulfentrazone | 140 | CHEAL | 21 | 98 |
| 1 | 19 | B2 | 560 | Sulfentrazone | 140 | CHEAL | 21 | 100 |
| 1 | 20 | B2 | 1120 | Sulfentrazone | 140 | CHEAL | 21 | 100 |
| 1 | 20 | B2 | 1120 | Sulfentrazone | 140 | CHEAL | 21 | 100 |
| 1 | 20 | B2 | 1120 | Sulfentrazone | 140 | CHEAL | 21 | 100 |
| 1 | 21 | B2 | 0 | Sulfentrazone | 210 | CHEAL | 21 | 100 |
| 1 | 21 | B2 | 0 | Sulfentrazone | 210 | CHEAL | 21 | 100 |
| 1 | 21 | B2 | 0 | Sulfentrazone | 210 | CHEAL | 21 | 99 |
| 1 | 22 | B2 | 375 | Sulfentrazone | 210 | CHEAL | 21 | 99 |
| 1 | 22 | B2 | 375 | Sulfentrazone | 210 | CHEAL | 21 | 100 |
| 1 | 22 | B2 | 375 | Sulfentrazone | 210 | CHEAL | 21 | 100 |
| 1 | 23 | B2 | 560 | Sulfentrazone | 210 | CHEAL | 21 | 100 |
| 1 | 23 | B2 | 560 | Sulfentrazone | 210 | CHEAL | 21 | 100 |
| 1 | 23 | B2 | 560 | Sulfentrazone | 210 | CHEAL | 21 | 100 |
| 1 | 24 | B2 | 1120 | Sulfentrazone | 210 | CHEAL | 21 | 98 |
| 1 | 24 | B2 | 1120 | Sulfentrazone | 210 | CHEAL | 21 | 100 |
| 1 | 24 | B2 | 1120 | Sulfentrazone | 210 | CHEAL | 21 | 100 |
| 1 | 25 | B2 | 0 | Sulfentrazone | 420 | CHEAL | 21 | 99 |
| 1 | 25 | B2 | 0 | Sulfentrazone | 420 | CHEAL | 21 | 98 |
| 1 | 25 | B2 | 0 | Sulfentrazone | 420 | CHEAL | 21 | 100 |
| 1 | 26 | B2 | 375 | Sulfentrazone | 420 | CHEAL | 21 | 99 |
| 1 | 26 | B2 | 375 | Sulfentrazone | 420 | CHEAL | 21 | 100 |
| 1 | 26 | B2 | 375 | Sulfentrazone | 420 | CHEAL | 21 | 100 |
| 1 | 27 | B2 | 560 | Sulfentrazone | 420 | CHEAL | 21 | 100 |
| 1 | 27 | B2 | 560 | Sulfentrazone | 420 | CHEAL | 21 | 100 |
| 1 | 27 | B2 | 560 | Sulfentrazone | 420 | CHEAL | 21 | 100 |
| 1 | 28 | B2 | 1120 | Sulfentrazone | 420 | CHEAL | 21 | 100 |
| 1 | 28 | B2 | 1120 | Sulfentrazone | 420 | CHEAL | 21 | 100 |
| 1 | 28 | B2 | 1120 | Sulfentrazone | 420 | CHEAL | 21 | 100 |
| 1 | 29 | B2 | 0 | Glyphosate | 375 | CHEAL | 21 | 50 |
| 1 | 29 | B2 | 0 | Glyphosate | 375 | CHEAL | 21 | 55 |
| 1 | 29 | B2 | 0 | Glyphosate | 375 | CHEAL | 21 | 55 |
| 1 | 30 | B2 | 375 | Glyphosate | 375 | CHEAL | 21 | 85 |
| 1 | 30 | B2 | 375 | Glyphosate | 375 | CHEAL | 21 | 100 |
| 1 | 30 | B2 | 375 | Glyphosate | 375 | CHEAL | 21 | 98 |
| 1 | 31 | B2 | 560 | Glyphosate | 375 | CHEAL | 21 | 90 |
| 1 | 31 | B2 | 560 | Glyphosate | 375 | CHEAL | 21 | 97 |
| 1 | 31 | B2 | 560 | Glyphosate | 375 | CHEAL | 21 | 98 |
| 1 | 32 | B2 | 1120 | Glyphosate | 375 | CHEAL | 21 | 90 |
| 1 | 32 | B2 | 1120 | Glyphosate | 375 | CHEAL | 21 | 80 |
| 1 | 32 | B2 | 1120 | Glyphosate | 375 | CHEAL | 21 | 97 |
| 1 | 33 | B2 | 0 | Glyphosate | 1120 | CHEAL | 21 | 65 |
| 1 | 33 | B2 | 0 | Glyphosate | 1120 | CHEAL | 21 | 70 |
| 1 | 33 | B2 | 0 | Glyphosate | 1120 | CHEAL | 21 | 85 |
| 1 | 34 | B2 | 375 | Glyphosate | 1120 | CHEAL | 21 | 95 |
| 1 | 34 | B2 | 375 | Glyphosate | 1120 | CHEAL | 21 | 98 |
| 1 | 34 | B2 | 375 | Glyphosate | 1120 | CHEAL | 21 | 98 |
| 1 | 35 | B2 | 560 | Glyphosate | 1120 | CHEAL | 21 | 95 |
| 1 | 35 | B2 | 560 | Glyphosate | 1120 | CHEAL | 21 | 100 |
| 1 | 35 | B2 | 560 | Glyphosate | 1120 | CHEAL | 21 | 97 |
| 1 | 36 | B2 | 1120 | Glyphosate | 1120 | CHEAL | 21 | 98 |
| 1 | 36 | B2 | 1120 | Glyphosate | 1120 | CHEAL | 21 | 98 |
| 1 | 36 | B2 | 1120 | Glyphosate | 1120 | CHEAL | 21 | 100 |
| 1 | 37 | B2 | 0 | Glyphosate | 1680 | CHEAL | 21 | 85 |
| 1 | 37 | B2 | 0 | Glyphosate | 1680 | CHEAL | 21 | 75 |
| 1 | 37 | B2 | 0 | Glyphosate | 1680 | CHEAL | 21 | 90 |
| 1 | 38 | B2 | 375 | Glyphosate | 1680 | CHEAL | 21 | 90 |
| 1 | 38 | B2 | 375 | Glyphosate | 1680 | CHEAL | 21 | 100 |
| 1 | 38 | B2 | 375 | Glyphosate | 1680 | CHEAL | 21 | 98 |
| 1 | 39 | B2 | 560 | Glyphosate | 1680 | CHEAL | 21 | 95 |
| 1 | 39 | B2 | 560 | Glyphosate | 1680 | CHEAL | 21 | 98 |
| 1 | 39 | B2 | 560 | Glyphosate | 1680 | CHEAL | 21 | 99 |
| 1 | 40 | B2 | 1120 | Glyphosate | 1680 | CHEAL | 21 | 98 |
| 1 | 40 | B2 | 1120 | Glyphosate | 1680 | CHEAL | 21 | 97 |
| 1 | 40 | B2 | 1120 | Glyphosate | 1680 | CHEAL | 21 | 98 |
| 1 | 41 | Untreated | | | | CHEAL | 21 | 0 |
| 1 | 41 | Untreated | | | | CHEAL | 21 | 0 |
| 1 | 41 | Untreated | | | | CHEAL | 21 | 0 |
| 1 | 1 | Solv. Ck | 0 | none | 0 | ERICA | 21 | 0 |
| | | untreated | | | | | | |
| 1 | 1 | Solv. Ck | | none | 0 | ERICA | 21 | 0 |
| | | untreated | | | | | | |
| 1 | 1 | Solv. Ck | 0 | none | 0 | ERICA | 21 | 0 |
| | | untreated | | | | | | |
| 1 | 2 | B2 | 375 | none | 0 | ERICA | 21 | 70 |
| 1 | 2 | B2 | 375 | none | 0 | ERICA | 21 | 95 |
| 1 | 2 | B2 | 375 | none | 0 | ERICA | 21 | 75 |
| 1 | 3 | B2 | 560 | none | 0 | ERICA | 21 | 75 |
| 1 | 3 | B2 | 560 | none | 0 | ERICA | 21 | 70 |
| 1 | 3 | B2 | 560 | none | 0 | ERICA | 21 | 55 |
| 1 | 4 | B2 | 1120 | none | 0 | ERICA | 21 | 85 |
| 1 | 4 | B2 | 1120 | none | 0 | ERICA | 21 | 75 |
| 1 | 4 | B2 | 1120 | none | 0 | ERICA | 21 | 85 |
| 1 | 5 | B2 | 0 | Saflufenacil | 25 | ERICA | 21 | 50 |
| 1 | 5 | B2 | 0 | Saflufenacil | 25 | ERICA | 21 | 25 |
| 1 | 5 | B2 | 0 | Saflufenacil | 25 | ERICA | 21 | 30 |
| 1 | 6 | B2 | 375 | Saflufenacil | 25 | ERICA | 21 | 25 |
| 1 | 6 | B2 | 375 | Saflufenacil | 25 | ERICA | 21 | 30 |
| 1 | 6 | B2 | 375 | Saflufenacil | 25 | ERICA | 21 | 35 |
| 1 | 7 | B2 | 560 | Saflufenacil | 25 | ERICA | 21 | 30 |
| 1 | 7 | B2 | 560 | Saflufenacil | 25 | ERICA | 21 | 35 |
| 1 | 7 | B2 | 560 | Saflufenacil | 25 | ERICA | 21 | 35 |
| 1 | 8 | B2 | 1120 | Saflufenacil | 25 | ERICA | 21 | 25 |
| 1 | 8 | B2 | 1120 | Saflufenacil | 25 | ERICA | 21 | 25 |
| 1 | 8 | B2 | 1120 | Saflufenacil | 25 | ERICA | 21 | 25 |
| 1 | 9 | B2 | 0 | Saflufenacil | 50 | ERICA | 21 | 100 |
| 1 | 9 | B2 | 0 | Saflufenacil | 50 | ERICA | 21 | 30 |
| 1 | 9 | B2 | 0 | Saflufenacil | 50 | ERICA | 21 | 30 |
| 1 | 10 | B2 | 375 | Saflufenacil | 50 | ERICA | 21 | 35 |
| 1 | 10 | B2 | 375 | Saflufenacil | 50 | ERICA | 21 | 100 |
| 1 | 10 | B2 | 375 | Saflufenacil | 50 | ERICA | 21 | 35 |
| 1 | 11 | B2 | 560 | Saflufenacil | 50 | ERICA | 21 | 35 |
| 1 | 11 | B2 | 560 | Saflufenacil | 50 | ERICA | 21 | 55 |
| 1 | 11 | B2 | 560 | Saflufenacil | 50 | ERICA | 21 | 50 |
| 1 | 12 | B2 | 1120 | Saflufenacil | 50 | ERICA | 21 | 50 |
| 1 | 12 | B2 | 1120 | Saflufenacil | 50 | ERICA | 21 | 75 |
| 1 | 12 | B2 | 1120 | Saflufenacil | 50 | ERICA | 21 | 45 |
| 1 | 13 | B2 | 0 | Saflufenacil | 75 | ERICA | 21 | 65 |
| 1 | 13 | B2 | 0 | Saflufenacil | 75 | ERICA | 21 | 50 |
| 1 | 13 | B2 | 0 | Saflufenacil | 75 | ERICA | 21 | 100 |
| 1 | 14 | B2 | 375 | Saflufenacil | 75 | ERICA | 21 | 40 |
| 1 | 14 | B2 | 375 | Saflufenacil | 75 | ERICA | 21 | 100 |
| 1 | 14 | B2 | 375 | Saflufenacil | 75 | ERICA | 21 | 50 |
| 1 | 15 | B2 | 560 | Saflufenacil | 75 | ERICA | 21 | 50 |
| 1 | 15 | B2 | 560 | Saflufenacil | 75 | ERICA | 21 | 50 |
| 1 | 15 | B2 | 560 | Saflufenacil | 75 | ERICA | 21 | 85 |
| 1 | 16 | B2 | 1120 | Saflufenacil | 75 | ERICA | 21 | 50 |
| 1 | 16 | B2 | 1120 | Saflufenacil | 75 | ERICA | 21 | 60 |
| 1 | 16 | B2 | 1120 | Saflufenacil | 75 | ERICA | 21 | 50 |
| 1 | 17 | B2 | 0 | Sulfentrazone | 140 | ERICA | 21 | 10 |
| 1 | 17 | B2 | 0 | Sulfentrazone | 140 | ERICA | 21 | 15 |
| 1 | 17 | B2 | 0 | Sulfentrazone | 140 | ERICA | 21 | 10 |
| 1 | 18 | B2 | 375 | Sulfentrazone | 140 | ERICA | 21 | 85 |
| 1 | 18 | B2 | 375 | Sulfentrazone | 140 | ERICA | 21 | 85 |
| 1 | 18 | B2 | 375 | Sulfentrazone | 140 | ERICA | 21 | 80 |
| 1 | 19 | B2 | 560 | Sulfentrazone | 140 | ERICA | 21 | 90 |
| 1 | 19 | B2 | 560 | Sulfentrazone | 140 | ERICA | 21 | 85 |
| 1 | 19 | B2 | 560 | Sulfentrazone | 140 | ERICA | 21 | 80 |
| 1 | 20 | B2 | 1120 | Sulfentrazone | 140 | ERICA | 21 | 85 |
| 1 | 20 | B2 | 1120 | Sulfentrazone | 140 | ERICA | 21 | 80 |
| 1 | 20 | B2 | 1120 | Sulfentrazone | 140 | ERICA | 21 | 80 |
| 1 | 21 | B2 | 0 | Sulfentrazone | 210 | ERICA | 21 | 15 |
| 1 | 21 | B2 | 0 | Sulfentrazone | 210 | ERICA | 21 | 15 |
| 1 | 21 | B2 | 0 | Sulfentrazone | 210 | ERICA | 21 | 10 |
| 1 | 22 | B2 | 375 | Sulfentrazone | 210 | ERICA | 21 | 85 |
| 1 | 22 | B2 | 375 | Sulfentrazone | 210 | ERICA | 21 | 90 |
| 1 | 22 | B2 | 375 | Sulfentrazone | 210 | ERICA | 21 | 80 |
| 1 | 23 | B2 | 560 | Sulfentrazone | 210 | ERICA | 21 | 90 |
| 1 | 23 | B2 | 560 | Sulfentrazone | 210 | ERICA | 21 | 95 |
| 1 | 23 | B2 | 560 | Sulfentrazone | 210 | ERICA | 21 | 85 |
| 1 | 24 | B2 | 1120 | Sulfentrazone | 210 | ERICA | 21 | 90 |
| 1 | 24 | B2 | 1120 | Sulfentrazone | 210 | ERICA | 21 | 85 |
| 1 | 24 | B2 | 1120 | Sulfentrazone | 210 | ERICA | 21 | 90 |
| 1 | 25 | B2 | 0 | Sulfentrazone | 420 | ERICA | 21 | 95 |
| 1 | 25 | B2 | 0 | Sulfentrazone | 420 | ERICA | 21 | 15 |
| 1 | 25 | B2 | 0 | Sulfentrazone | 420 | ERICA | 21 | 15 |
| 1 | 26 | B2 | 375 | Sulfentrazone | 420 | ERICA | 21 | 100 |
| 1 | 26 | B2 | 375 | Sulfentrazone | 420 | ERICA | 21 | 75 |
| 1 | 26 | B2 | 375 | Sulfentrazone | 420 | ERICA | 21 | 80 |
| 1 | 27 | B2 | 560 | Sulfentrazone | 420 | ERICA | 21 | 99 |
| 1 | 27 | B2 | 560 | Sulfentrazone | 420 | ERICA | 21 | 85 |
| 1 | 27 | B2 | 560 | Sulfentrazone | 420 | ERICA | 21 | 80 |
| 1 | 28 | B2 | 1120 | Sulfentrazone | 420 | ERICA | 21 | 100 |
| 1 | 28 | B2 | 1120 | Sulfentrazone | 420 | ERICA | 21 | 80 |
| 1 | 28 | B2 | 1120 | Sulfentrazone | 420 | ERICA | 21 | 85 |
| 1 | 29 | B2 | 0 | Glyphosate | 375 | ERICA | 21 | 100 |
| 1 | 29 | B2 | 0 | Glyphosate | 375 | ERICA | 21 | 40 |
| 1 | 29 | B2 | 0 | Glyphosate | 375 | ERICA | 21 | 60 |
| 1 | 30 | B2 | 375 | Glyphosate | 375 | ERICA | 21 | 100 |
| 1 | 30 | B2 | 375 | Glyphosate | 375 | ERICA | 21 | 75 |
| 1 | 30 | B2 | 375 | Glyphosate | 375 | ERICA | 21 | 75 |
| 1 | 31 | B2 | 560 | Glyphosate | 375 | ERICA | 21 | 100 |
| 1 | 31 | B2 | 560 | Glyphosate | 375 | ERICA | 21 | 65 |
| 1 | 31 | B2 | 560 | Glyphosate | 375 | ERICA | 21 | 85 |
| 1 | 32 | B2 | 1120 | Glyphosate | 375 | ERICA | 21 | 100 |
| 1 | 32 | B2 | 1120 | Glyphosate | 375 | ERICA | 21 | 65 |
| 1 | 32 | B2 | 1120 | Glyphosate | 375 | ERICA | 21 | 80 |
| 1 | 33 | B2 | 0 | Glyphosate | 1120 | ERICA | 21 | 95 |
| 1 | 33 | B2 | 0 | Glyphosate | 1120 | ERICA | 21 | 100 |
| 1 | 33 | B2 | 0 | Glyphosate | 1120 | ERICA | 21 | 100 |
| 1 | 34 | B2 | 375 | Glyphosate | 1120 | ERICA | 21 | 100 |
| 1 | 34 | B2 | 375 | Glyphosate | 1120 | ERICA | 21 | 100 |
| 1 | 34 | B2 | 375 | Glyphosate | 1120 | ERICA | 21 | 100 |
| 1 | 35 | B2 | 560 | Glyphosate | 1120 | ERICA | 21 | 100 |
| 1 | 35 | B2 | 560 | Glyphosate | 1120 | ERICA | 21 | 98 |
| 1 | 35 | B2 | 560 | Glyphosate | 1120 | ERICA | 21 | 100 |
| 1 | 36 | B2 | 1120 | Glyphosate | 1120 | ERICA | 21 | 100 |
| 1 | 36 | B2 | 1120 | Glyphosate | 1120 | ERICA | 21 | 100 |
| 1 | 36 | B2 | 1120 | Glyphosate | 1120 | ERICA | 21 | 100 |
| 1 | 37 | B2 | 0 | Glyphosate | 1680 | ERICA | 21 | 100 |
| 1 | 37 | B2 | 0 | Glyphosate | 1680 | ERICA | 21 | 100 |
| 1 | 37 | B2 | 0 | Glyphosate | 1680 | ERICA | 21 | 100 |
| 1 | 38 | B2 | 375 | Glyphosate | 1680 | ERICA | 21 | 100 |
| 1 | 38 | B2 | 375 | Glyphosate | 1680 | ERICA | 21 | 100 |
| 1 | 38 | B2 | 375 | Glyphosate | 1680 | ERICA | 21 | 100 |
| 1 | 39 | B2 | 560 | Glyphosate | 1680 | ERICA | 21 | 100 |
| 1 | 39 | B2 | 560 | Glyphosate | 1680 | ERICA | 21 | 100 |
| 1 | 39 | B2 | 560 | Glyphosate | 1680 | ERICA | 21 | 100 |
| 1 | 40 | B2 | 1120 | Glyphosate | 1680 | ERICA | 21 | 100 |
| 1 | 40 | B2 | 1120 | Glyphosate | 1680 | ERICA | 21 | 100 |
| 1 | 40 | B2 | 1120 | Glyphosate | 1680 | ERICA | 21 | 100 |
| 1 | 41 | Untreated | | | | ERICA | 21 | 0 |
| 1 | 41 | Untreated | | | | ERICA | 21 | 0 |
| 1 | 41 | Untreated | | | | ERICA | 21 | 0 |
| 1 | 1 | Solv. Ck | 0 | none | 0 | ZEAMX | 21 | 0 |
| | | untreated | | | | | | |
| 1 | 1 | Solv. Ck | 0 | none | 0 | ZEAMX | 21 | 0 |
| | | untreated | | | | | | |
| 1 | 1 | Solv. Ck | 0 | none | 0 | ZEAMX | 21 | 0 |
| | | untreated | | | | | | |
| 1 | 2 | B2 | 375 | none | 0 | ZEAMX | 21 | 3 |
| 1 | 2 | B2 | 375 | none | 0 | ZEAMX | 21 | 3 |
| 1 | 2 | B2 | 375 | none | 0 | ZEAMX | 21 | 3 |
| 1 | 3 | B2 | 560 | none | 0 | ZEAMX | 21 | 5 |
| 1 | 3 | B2 | 560 | none | 0 | ZEAMX | 21 | 5 |
| 1 | 3 | B2 | 560 | none | 0 | ZEAMX | 21 | 15 |
| 1 | 4 | B2 | 1120 | none | 0 | ZEAMX | 21 | 5 |
| 1 | 4 | B2 | 1120 | none | 0 | ZEAMX | 21 | 5 |
| 1 | 4 | B2 | 1120 | none | 0 | ZEAMX | 21 | 5 |
| 1 | 5 | B2 | 0 | Saflufenacil | 25 | ZEAMX | 21 | 30 |
| 1 | 5 | B2 | 0 | Saflufenacil | 25 | ZEAMX | 21 | 50 |
| 1 | 5 | B2 | 0 | Saflufenacil | 25 | ZEAMX | 21 | 20 |
| 1 | 6 | B2 | 375 | Saflufenacil | 25 | ZEAMX | 21 | 20 |
| 1 | 6 | B2 | 375 | Saflufenacil | 25 | ZEAMX | 21 | 35 |
| 1 | 6 | B2 | 375 | Saflufenacil | 25 | ZEAMX | 21 | 30 |
| 1 | 7 | B2 | 560 | Saflufenacil | 25 | ZEAMX | 21 | 30 |
| 1 | 7 | B2 | 560 | Saflufenacil | 25 | ZEAMX | 21 | 25 |
| 1 | 7 | B2 | 560 | Saflufenacil | 25 | ZEAMX | 21 | 40 |
| 1 | 8 | B2 | 1120 | Saflufenacil | 25 | ZEAMX | 21 | 15 |
| 1 | 8 | B2 | 1120 | Saflufenacil | 25 | ZEAMX | 21 | 20 |
| 1 | 8 | B2 | 1120 | Saflufenacil | 25 | ZEAMX | 21 | 30 |
| 1 | 9 | B2 | 0 | Saflufenacil | 50 | ZEAMX | 21 | 50 |
| 1 | 9 | B2 | 0 | Saflufenacil | 50 | ZEAMX | 21 | 45 |
| 1 | 9 | B2 | 0 | Saflufenacil | 50 | ZEAMX | 21 | 35 |
| 1 | 10 | B2 | 375 | Saflufenacil | 50 | ZEAMX | 21 | 55 |
| 1 | 10 | B2 | 375 | Saflufenacil | 50 | ZEAMX | 21 | 60 |
| 1 | 10 | B2 | 375 | Saflufenacil | 50 | ZEAMX | 21 | 50 |
| 1 | 11 | B2 | $60 | Saflufenacil | 50 | ZEAMX | 21 | 60 |
| 1 | 11 | B2 | 560 | Saflufenacil | 50 | ZEAMX | 21 | 35 |
| 1 | 11 | B2 | 560 | Saflufenacil | 50 | ZEAMX | 21 | 35 |
| 1 | 12 | B2 | 1120 | Saflufenacil | 50 | ZEAMX | 21 | 30 |
| 1 | 12 | B2 | 1120 | Saflufenacil | 50 | ZEAMX | 21 | 60 |
| 1 | 12 | B2 | 1120 | Saflufenacil | 50 | ZEAMX | 21 | 50 |
| 1 | 13 | B2 | 0 | Saflufenacil | 75 | ZEAMX | 21 | 45 |
| 1 | 13 | B2 | 0 | Saflufenacil | 75 | ZEAMX | 21 | 50 |
| 1 | 13 | B2 | 0 | Saflufenacil | 75 | ZEAMX | 21 | 45 |
| 1 | 14 | B2 | 375 | Saflufenacil | 75 | ZEAMX | 21 | 65 |
| 1 | 14 | B2 | 375 | Saflufenacil | 75 | ZEAMX | 21 | 55 |
| 1 | 14 | B2 | 375 | Saflufenacil | 75 | ZEAMX | 21 | 60 |
| 1 | 15 | B2 | 560 | Saflufenacil | 75 | ZEAMX | 21 | 60 |
| 1 | 15 | B2 | 560 | Saflufenacil | 75 | ZEAMX | 21 | 55 |
| 1 | 15 | B2 | 560 | Saflufenacil | 75 | ZEAMX | 21 | 65 |
| 1 | 16 | B2 | 1120 | Saflufenacil | 75 | ZEAMX | 21 | 55 |
| 1 | 16 | B2 | 1120 | Saflufenacil | 75 | ZEAMX | 21 | 45 |
| 1 | 16 | B2 | 1120 | Saflufenacil | 75 | ZEAMX | 21 | 65 |
| 1 | 17 | B2 | 0 | Sulfentrazone | 140 | ZEAMX | 21 | 10 |
| 1 | 17 | B2 | 0 | Sulfentrazone | 140 | ZEAMX | 21 | 5 |
| 1 | 17 | B2 | 0 | Sulfentrazone | 140 | ZEAMX | 21 | 30 |
| 1 | 18 | B2 | 375 | Sulfentrazone | 140 | ZEAMX | 21 | 5 |
| 1 | 18 | B2 | 375 | Sulfentrazone | 140 | ZEAMX | 21 | 10 |
| 1 | 18 | B2 | 375 | Sulfentrazone | 140 | ZEAMX | 21 | 10 |
| 1 | 19 | B2 | 560 | Sulfentrazone | 140 | ZEAMX | 21 | 5 |
| 1 | 19 | B2 | 560 | Sulfentrazone | 140 | ZEAMX | 21 | 10 |
| 1 | 19 | B2 | 560 | Sulfentrazone | 140 | ZEAMX | 21 | 30 |
| 1 | 20 | B2 | 1120 | Sulfentrazone | 140 | ZEAMX | 21 | 25 |
| 1 | 20 | B2 | 1120 | Sulfentrazone | 140 | ZEAMX | 21 | 30 |
| 1 | 20 | B2 | 1120 | Sulfentrazone | 140 | ZEAMX | 21 | 40 |
| 1 | 21 | B2 | 0 | Sulfentrazone | 210 | ZEAMX | 21 | 30 |
| 1 | 21 | B2 | 0 | Sulfentrazone | 210 | ZEAMX | 21 | 20 |
| 1 | 21 | B2 | 0 | Sulfentrazone | 210 | ZEAMX | 21 | 50 |
| 1 | 22 | B2 | 375 | Sulfentrazone | 210 | ZEAMX | 21 | 20 |
| 1 | 22 | B2 | 375 | Sulfentrazone | 210 | ZEAMX | 21 | 35 |
| 1 | 22 | B2 | 375 | Sulfentrazone | 210 | ZEAMX | 21 | 35 |
| 1 | 23 | B2 | 560 | Sulfentrazone | 210 | ZEAMX | 21 | 50 |
| 1 | 23 | B2 | 560 | Sulfentrazone | 210 | ZEAMX | 21 | 15 |
| 1 | 23 | B2 | 560 | Sulfentrazone | 210 | ZEAMX | 21 | 35 |
| 1 | 24 | B2 | 1120 | Sulfentrazone | 210 | ZEAMX | 21 | 25 |
| 1 | 24 | B2 | 1120 | Sulfentrazone | 210 | ZEAMX | 21 | 25 |
| 1 | 24 | B2 | 1120 | Sulfentrazone | 210 | ZEAMX | 21 | 65 |
| 1 | 25 | B2 | 0 | Sulfentrazone | 420 | ZEAMX | 21 | 25 |
| 1 | 25 | B2 | 0 | Sulfentrazone | 420 | ZEAMX | 21 | 30 |
| 1 | 25 | B2 | 0 | Sulfentrazone | 420 | ZEAMX | 21 | 30 |
| 1 | 26 | B2 | 375 | Sulfentrazone | 420 | ZEAMX | 21 | 35 |
| 1 | 26 | B2 | 375 | Sulfentrazone | 420 | ZEAMX | 21 | 30 |
| 1 | 26 | B2 | 375 | Sulfentrazone | 420 | ZEAMX | 21 | 60 |
| 1 | 27 | B2 | 560 | Sulfentrazone | 420 | ZEAMX | 21 | 35 |
| 1 | 27 | B2 | 560 | Sulfentrazone | 420 | ZEAMX | 21 | 55 |
| 1 | 27 | B2 | 560 | Sulfentrazone | 420 | ZEAMX | 21 | 55 |
| 1 | 28 | B2 | 1120 | Sulfentrazone | 420 | ZEAMX | 21 | 50 |
| 1 | 28 | B2 | 1120 | Sulfentrazone | 420 | ZEAMX | 21 | 40 |
| 1 | 28 | B2 | 1120 | Sulfentrazone | 420 | ZEAMX | 21 | 65 |
| 1 | 29 | B2 | 0 | Glyphosate | 375 | ZEAMX | 21 | 70 |
| 1 | 29 | B2 | 0 | Glyphosate | 375 | ZEAMX | 21 | 65 |
| 1 | 29 | B2 | 0 | Glyphosate | 375 | ZEAMX | 21 | 80 |
| 1 | 30 | B2 | 375 | Glyphosate | 375 | ZEAMX | 21 | 55 |
| 1 | 30 | B2 | 375 | Glyphosate | 375 | ZEAMX | 21 | 75 |
| 1 | 30 | B2 | 375 | Glyphosate | 375 | ZEAMX | 21 | 80 |
| 1 | 31 | B2 | 560 | Glyphosate | 375 | ZEAMX | 21 | 20 |
| 1 | 31 | B2 | 560 | Glyphosate | 375 | ZEAMX | 21 | 45 |
| 1 | 31 | B2 | 560 | Glyphosate | 375 | ZEAMX | 21 | 50 |
| 1 | 32 | B2 | 1120 | Glyphosate | 375 | ZEAMX | 21 | 60 |
| 1 | 32 | B2 | 1120 | Glyphosate | 375 | ZEAMX | 21 | 45 |
| 1 | 32 | B2 | 1120 | Glyphosate | 375 | ZEAMX | 21 | 80 |
| 1 | 33 | B2 | 0 | Glyphosate | 1120 | ZEAMX | 21 | 85 |
| 1 | 33 | B2 | 0 | Glyphosate | 1120 | ZEAMX | 21 | 90 |
| 1 | 33 | B2 | 0 | Glyphosate | 1120 | ZEAMX | 21 | 90 |
| 1 | 34 | B2 | 375 | Glyphosate | 1120 | ZEAMX | 21 | 80 |
| 1 | 34 | B2 | 375 | Glyphosate | 1120 | ZEAMX | 21 | 80 |
| 1 | 34 | B2 | 375 | Glyphosate | 1120 | ZEAMX | 21 | 90 |
| 1 | 35 | B2 | 560 | Glyphosate | 1120 | ZEAMX | 21 | 80 |
| 1 | 35 | B2 | 560 | Glyphosate | 1120 | ZEAMX | 21 | 90 |
| 1 | 35 | B2 | 560 | Glyphosate | 1120 | ZEAMX | 21 | 85 |
| 1 | 36 | B2 | 1120 | Glyphosate | 1120 | ZEAMX | 21 | 90 |
| 1 | 36 | B2 | 1120 | Glyphosate | 1120 | ZEAMX | 21 | 95 |
| 1 | 36 | B2 | 1120 | Glyphosate | 1120 | ZEAMX | 21 | 85 |
| 1 | 37 | B2 | 0 | Glyphosate | 1680 | ZEAMX | 21 | 100 |
| 1 | 37 | B2 | 0 | Glyphosate | 1680 | ZEAMX | 21 | 100 |
| 1 | 37 | B2 | 0 | Glyphosate | 1680 | ZEAMX | 21 | 00 |
| 1 | 38 | B2 | 375 | Glyphosate | 1680 | ZEAMX | 21 | 95 |
| 1 | 38 | B2 | 375 | Glyphosate | 1680 | ZEAMX | 21 | 98 |
| 1 | 38 | B2 | 375 | Glyphosate | 1680 | ZEAMX | 21 | 100 |
| 1 | 39 | B2 | 560 | Glyphosate | 1680 | ZEAMX | 21 | 98 |
| 1 | 39 | B2 | 560 | Glyphosate | 1680 | ZEAMX | 21 | 97 |
| 1 | 39 | B2 | 560 | Glyphosate | 1680 | ZEAMX | 21 | 97 |
| 1 | 40 | B2 | 1120 | Glyphosate | 1680 | ZEAMX | 21 | 100 |
| 1 | 40 | B2 | 1120 | Glyphosate | 1680 | ZEAMX | 21 | 100 |
| 1 | 40 | B2 | 1120 | Glyphosate | 1680 | ZEAMX | 21 | 98 |
| 1 | 41 | Untreated | | | | ZEAMX | 21 | 0 |
| 1 | 41 | Untreated | | | | ZEAMX | 21 | 0 |
| 1 | 41 | Untreated | | | | ZEAMX | 21 | 0 |
| 2 | 1 | Solv. Ck | 0 | none | 0 | ABUTH | 21 | 0 |
| | | untreated | | | | | | |
| 2 | 1 | Solv. Ck | 0 | none | 0 | ABUTH | 21 | 0 |
| | | untreated | | | | | | |
| 2 | 1 | Solv. Ck | 0 | none | 0 | ABUTH | 21 | 5 |
| | | untreated | | | | | | |
| 2 | 2 | B2 | 560 | none | | ABUTH | 21 | 80 |
| 2 | 2 | B2 | 560 | none | | ABUTH | 21 | 98 |
| 2 | 2 | B2 | 560 | none | | ABUTH | 21 | 88 |
| 2 | 3 | B2 | 1120 | none | | ABUTH | 21 | 95 |
| 2 | 3 | B2 | 1120 | none | | ABUTH | 21 | 99 |
| 2 | 3 | B2 | 1120 | none | | ABUTH | 21 | 98 |
| 2 | 4 | B2 | 0 | Glufosinate | 200 | ABUTH | 21 | 5 |
| 2 | 4 | B2 | 0 | Glufosinate | 200 | ABUTH | 21 | 10 |
| 2 | 4 | B2 | 0 | Glufosinate | 200 | ABUTH | 21 | 10 |
| 2 | 5 | B2 | 560 | Glufosinate | 200 | ABUTH | 21 | 90 |
| 2 | 5 | B2 | 560 | Glufosinate | 200 | ABUTH | 21 | 85 |
| 2 | 5 | B2 | 560 | Glufosinate | 200 | ABUTH | 21 | 92 |
| 2 | 6 | B2 | 1120 | Glufosinate | 200 | ABUTH | 21 | 90 |
| 2 | 6 | B2 | 1120 | Glufosinate | 200 | ABUTH | 21 | 88 |
| 2 | 6 | B2 | 1120 | Glufosinate | 200 | ABUTH | 21 | 92 |
| 2 | 7 | B2 | 0 | Glufosinate | 600 | ABUTH | 21 | 35 |
| 2 | 7 | B2 | 0 | Glufosinate | 600 | ABUTH | 21 | 65 |
| 2 | 7 | B2 | 0 | Glufosinate | 600 | ABUTH | 21 | 100 |
| 2 | 8 | B2 | 560 | Glufosinate | 600 | ABUTH | 21 | 80 |
| 2 | 8 | B2 | 560 | Glufosinate | 600 | ABUTH | 21 | 98 |
| 2 | 8 | B2 | 560 | Glufosinate | 600 | ABUTH | 21 | 00 |
| 2 | 9 | B2 | 1120 | Glufosinate | 600 | ABUTH | 21 | 90 |
| 2 | 9 | B2 | 1120 | Glufosinate | 600 | ABUTH | 21 | 97 |
| 2 | 9 | B2 | 1120 | Glufosinate | 600 | ABUTH | 21 | 99 |
| 2 | 10 | B2 | 0 | Glufosinate | 900 | ABUTH | 21 | 78 |
| 2 | 10 | B2 | 0 | Glufosinate | 900 | ABUTH | 21 | 55 |
| 2 | 10 | B2 | 0 | Glufosinate | 900 | ABUTH | 21 | 60 |
| 2 | 11 | B2 | 560 | Glufosinate | 900 | ABUTH | 21 | 90 |
| 2 | 11 | B2 | 560 | Glufosinate | 900 | ABUTH | 21 | 99 |
| 2 | 11 | B2 | 560 | Glufosinate | 900 | ABUTH | 21 | 99 |
| 2 | 12 | B2 | 1120 | Glufosinate | 900 | ABUTH | 21 | 99 |
| 2 | 12 | B2 | 1120 | Glufosinate | 900 | ABUTH | 21 | 99 |
| 2 | 12 | B2 | 1120 | Glufosinate | 900 | ABUTH | 21 | 100 |
| 2 | 13 | B2 | 0 | Diclosulam | 25 | ABUTH | 21 | 100 |
| 2 | 13 | B2 | 0 | Diclosulam | 25 | ABUTH | 21 | 100 |
| 2 | 13 | B2 | 0 | Diclosulam | 25 | ABUTH | 21 | 98 |
| 2 | 14 | B2 | 560 | Diclosulam | 25 | ABUTH | 21 | 99 |
| 2 | 14 | B2 | 560 | Diclosulam | 25 | ABUTH | 21 | 97 |
| 2 | 14 | B2 | 560 | Diclosulam | 25 | ABUTH | 21 | 95 |
| 2 | 15 | B2 | 1120 | Diclosulam | 25 | ABUTH | 21 | 99 |
| 2 | 15 | B2 | 1120 | Diclosulam | 25 | ABUTH | 21 | 100 |
| 2 | 15 | B2 | 1120 | Diclosulam | 25 | ABUTH | 21 | 97 |
| 2 | 16 | B2 | 0 | Diclosulam | 35 | ABUTH | 21 | 98 |
| 2 | 16 | B2 | 0 | Diclosulam | 35 | ABUTH | 21 | 100 |
| 2 | 16 | B2 | 0 | Diclosulam | 35 | ABUTH | 21 | 100 |
| 2 | 17 | B2 | 560 | Diclosulam | 35 | ABUTH | 21 | 100 |
| 2 | 17 | B2 | 560 | Diclosulam | 35 | ABUTH | 21 | 100 |
| 2 | 17 | B2 | 560 | Diclosulam | 35 | ABUTH | 21 | 100 |
| 2 | 18 | B2 | 1120 | Diclosulam | 35 | ABUTH | 21 | 99 |
| 2 | 18 | B2 | 1120 | Diclosulam | 35 | ABUTH | 21 | 100 |
| 2 | 18 | B2 | 1120 | Diclosulam | 35 | ABUTH | 21 | 100 |
| 2 | 19 | B2 | 0 | Arylex | 5 | ABUTH | 21 | 100 |
| 2 | 19 | B2 | 0 | Arvlex | 5 | ABUTH | 21 | 100 |
| 2 | 19 | B2 | 0 | Arylex | 5 | ABUTH | 21 | 100 |
| 2 | 20 | B2 | 560 | Arvlex | 5 | ABUTH | 21 | 99 |
| 2 | 20 | B2 | 560 | Arylex | 5 | ABUTH | 21 | 99 |
| 2 | 20 | B2 | 560 | Arylex | 5 | ABUTH | 21 | 100 |
| 2 | 21 | B2 | 1120 | Arylex | 5 | ABUTH | 21 | 100 |
| 2 | 21 | B2 | 1120 | Arvlex | 5 | ABUTH | 21 | 99 |
| 2 | 21 | B2 | 1120 | Arylex | 5 | ABUTH | 21 | 100 |
| 2 | 22 | B2 | 0 | Arvlex | 65 | ABUTH | 21 | 100 |
| 2 | 22 | B2 | 0 | Arylex | 65 | ABUTH | 21 | 100 |
| 2 | 22 | B2 | 0 | Arylex | 65 | ABUTH | 21 | 100 |
| 2 | 23 | B2 | 560 | Arylex | 65 | ABUTH | 21 | 100 |
| 2 | 23 | B2 | 560 | Arvlex | 65 | ABUTH | 21 | 99 |
| 2 | 23 | B2 | 560 | Arylex | 65 | ABUTH | 21 | 100 |
| 2 | 24 | B2 | 1120 | Arylex | 65 | ABUTH | 21 | 99 |
| 2 | 24 | B2 | 1120 | Arylex | 65 | ABUTH | 21 | 100 |
| 2 | 24 | B2 | 1120 | Arylex | 65 | ABUTH | 21 | 100 |
| 2 | 25 | B2 | 0 | Mesotrione | 75 | ABUTH | 21 | 100 |
| 2 | 25 | B2 | 0 | Mesotrione | 75 | ABUTH | 21 | 100 |
| 2 | 25 | B2 | 0 | Mesotrione | 75 | ABUTH | 21 | 100 |
| 2 | 26 | B2 | 560 | Mesotrione | 75 | ABUTH | 21 | 100 |
| 2 | 26 | B2 | 560 | Mesotrione | 75 | ABUTH | 21 | 100 |
| 2 | 26 | B2 | 560 | Mesotrione | 75 | ABUTH | 21 | 100 |
| 2 | 27 | B2 | 1120 | Mesotrione | 75 | ABUTH | 21 | 100 |
| 2 | 27 | B2 | 1120 | Mesotrione | 75 | ABUTH | 21 | 100 |
| 2 | 27 | B2 | 1120 | Mesotrione | 75 | ABUTH | 21 | 100 |
| 2 | 28 | B2 | 0 | Mesotrione | 150 | ABUTH | 21 | 100 |
| 2 | 28 | B2 | 0 | Mesotrione | 150 | ABUTH | 21 | 100 |
| 2 | 28 | B2 | 0 | Mesotrione | 150 | ABUTH | 21 | 100 |
| 2 | 29 | B2 | 560 | Mesotrione | 150 | ABUTH | 21 | 100 |
| 2 | 29 | B2 | 560 | Mesotrione | 150 | ABUTH | 21 | 100 |
| 2 | 29 | B2 | 560 | Mesotrione | 150 | ABUTH | 21 | 100 |
| 2 | 30 | B2 | 1120 | Mesotrione | 150 | ABUTH | 21 | 100 |
| 2 | 30 | B2 | 1120 | Mesotrione | 150 | ABUTH | 21 | 100 |
| 2 | 30 | B2 | 1120 | Mesotrione | 150 | ABUTH | 21 | 100 |
| 2 | 31 | Untreated | | | | ABUTH | 21 | 0 |
| 2 | 31 | Untreated | | | | ABUTH | 21 | 0 |
| 2 | 31 | Untreated | | | | ABUTH | 21 | 0 |
| 2 | 1 | Solv. Ck | 0 | none | 0 | AMATA | 21 | 0 |
| | | untreated | | | | | | |
| 2 | 1 | Solv. Ck | 0 | none | 0 | AMATA | 21 | 0 |
| | | untreated | | | | | | |
| 2 | 1 | Solv. Ck | 0 | none | 0 | AMATA | 21 | 0 |
| | | untreated | | | | | | |
| 2 | 2 | B2 | 560 | none | | AMATA | 21 | 98 |
| 2 | 2 | B2 | 560 | none | | AMATA | 21 | 100 |
| 2 | 2 | B2 | 560 | none | | AMATA | 21 | 95 |
| 2 | 3 | B2 | 1120 | none | | AMATA | 21 | 100 |
| 2 | 3 | B2 | 1120 | none | | AMATA | 21 | 100 |
| 2 | 3 | B2 | 1120 | none | | AMATA | 21 | 100 |
| 2 | 4 | B2 | 0 | Glufosinate | 200 | AMATA | 21 | 70 |
| 2 | 4 | B2 | 0 | Glufosinate | 200 | AMATA | 21 | 50 |
| 2 | 4 | B2 | 0 | Glufosinate | 200 | AMATA | 21 | 100 |
| 2 | 5 | B2 | 560 | Glufosinate | 200 | AMATA | 21 | 95 |
| 2 | 5 | B2 | 560 | Glufosinate | 200 | AMATA | 21 | 50 |
| 2 | 5 | B2 | 560 | Glufosinate | 200 | AMATA | 21 | 100 |
| 2 | 6 | B2 | 1120 | Glufosinate | 200 | AMATA | 21 | 100 |
| 2 | 6 | B2 | 1120 | Glufosinate | 200 | AMATA | 21 | 100 |
| 2 | 6 | B2 | 1120 | Glufosinate | 200 | AMATA | 21 | 65 |
| 2 | 7 | B2 | 0 | Glufosinate | 600 | AMATA | 21 | 100 |
| 2 | 7 | B2 | 0 | Glufosinate | 600 | AMATA | 21 | 50 |
| 2 | 7 | B2 | 0 | Glufosinate | 600 | AMATA | 21 | 100 |
| 2 | 8 | B2 | 560 | Glufosinate | 600 | AMATA | 21 | 100 |
| 2 | 8 | B2 | 560 | Glufosinate | 600 | AMATA | 21 | 100 |
| 2 | 8 | B2 | 560 | Glufosinate | 600 | AMATA | 21 | 100 |
| 2 | 9 | B2 | 1120 | Glufosinate | 600 | AMATA | 21 | 100 |
| 2 | 9 | B2 | 1120 | Glufosinate | 600 | AMATA | 21 | 100 |
| 2 | 9 | B2 | 1120 | Glufosinate | 600 | AMATA | 21 | 100 |
| 2 | 10 | B2 | 0 | Glufosinate | 900 | AMATA | 21 | 100 |
| 2 | 10 | B2 | 0 | Glufosinate | 900 | AMATA | 21 | 100 |
| 2 | 10 | B2 | 0 | Glufosinate | 900 | AMATA | 21 | 100 |
| 2 | 11 | B2 | 560 | Glufosinate | 900 | AMATA | 21 | 100 |
| 2 | 11 | B2 | 560 | Glufosinate | 900 | AMATA | 21 | 100 |
| 2 | 11 | B2 | 560 | Glufosinate | 900 | AMATA | 21 | 100 |
| 2 | 12 | B2 | 1120 | Glufosinate | 900 | AMATA | 21 | 100 |
| 2 | 12 | B2 | 1120 | Glufosinate | 900 | AMATA | 21 | 100 |
| 2 | 12 | B2 | 1120 | Glufosinate | 900 | AMATA | 21 | 100 |
| 2 | 13 | B2 | 0 | Diclosulam | 25 | AMATA | 21 | 0 |
| 2 | 13 | B2 | 0 | Diclosulam | 25 | AMATA | 21 | 5 |
| 2 | 13 | B2 | 0 | Diclosulam | 25 | AMATA | 21 | 0 |
| 2 | 14 | B2 | 560 | Diclosulam | 25 | AMATA | 21 | 98 |
| 2 | 14 | B2 | 560 | Diclosulam | 25 | AMATA | 21 | 100 |
| 2 | 14 | B2 | 560 | Diclosulam | 25 | AMATA | 21 | 100 |
| 2 | 15 | B2 | 1120 | Diclosulam | 25 | AMATA | 21 | 100 |
| 2 | 15 | B2 | 1120 | Diclosulam | 25 | AMATA | 21 | 95 |
| 2 | 15 | B2 | 1120 | Diclosulam | 25 | AMATA | 21 | 100 |
| 2 | 16 | B2 | 0 | Diclosulam | 35 | AMATA | 21 | 30 |
| 2 | 16 | B2 | 0 | Diclosulam | 35 | AMATA | 21 | 20 |
| 2 | 16 | B2 | 0 | Diclosulam | 35 | AMATA | 21 | 5 |
| 2 | 17 | B2 | 560 | Diclosulam | 35 | AMATA | 21 | 97 |
| 2 | 17 | B2 | 560 | Diclosulam | 35 | AMATA | 21 | 92 |
| 2 | 17 | B2 | 560 | Diclosulam | 35 | AMATA | 21 | 100 |
| 2 | 18 | B2 | 1120 | Diclosulam | 35 | AMATA | 21 | 100 |
| 2 | 18 | B2 | 1120 | Diclosulam | 35 | AMATA | 21 | 95 |
| 2 | 18 | B2 | 1120 | Diclosulam | 35 | AMATA | 21 | 98 |
| 2 | 19 | B2 | 0 | Arylex | 5 | AMATA | 21 | 75 |
| 2 | 19 | B2 | 0 | Arvlex | 5 | AMATA | 21 | 92 |
| 2 | 19 | B2 | 0 | Arylex | 5 | AMATA | 21 | 75 |
| 2 | 20 | B2 | 560 | Arylex | 5 | AMATA | 21 | 99 |
| 2 | 20 | B2 | 560 | Arylex | 5 | AMATA | 21 | 100 |
| 2 | 20 | B2 | 560 | Arylex | 5 | AMATA | 21 | 100 |
| 2 | 21 | B2 | 1120 | Arylex | 5 | AMATA | 21 | 95 |
| 2 | 21 | B2 | 1120 | Arylex | 5 | AMATA | 21 | 100 |
| 2 | 21 | B2 | 1120 | Arvlex | 5 | AMATA | 21 | 100 |
| 2 | 22 | B2 | 0 | Arylex | 6.5 | AMATA | 21 | 99 |
| 2 | 22 | B2 | 0 | Arvlex | 6.5 | AMATA | 21 | 92 |
| 2 | 22 | B2 | 0 | Arylex | 6.5 | AMATA | 21 | 70 |
| 2 | 23 | B2 | 560 | Arylex | 6.5 | AMATA | 21 | 98 |
| 2 | 23 | B2 | 560 | Arylex | 6.5 | AMATA | 21 | 100 |
| 2 | 23 | B2 | 560 | Arylex | 6.5 | AMATA | 21 | 100 |
| 2 | 24 | B2 | 1120 | Arylex | 6.5 | AMATA | 21 | 100 |
| 2 | 24 | B2 | 1120 | Arvlex | 6.5 | AMATA | 21 | 100 |
| 2 | 24 | B2 | 1120 | Arylex | 6.5 | AMATA | 21 | 100 |
| 2 | 25 | B2 | 0 | Mesotrione | 75 | AMATA | 21 | 100 |
| 2 | 25 | B2 | 0 | Mesotrione | 75 | AMATA | 21 | 100 |
| 2 | 25 | B2 | 0 | Mesotrione | 75 | AMATA | 21 | 100 |
| 2 | 26 | B2 | 560 | Mesotrione | 75 | AMATA | 21 | 100 |
| 2 | 26 | B2 | 560 | Mesotrione | 75 | AMATA | 21 | 100 |
| 2 | 26 | B2 | 560 | Mesotrione | 75 | AMATA | 21 | 100 |
| 2 | 27 | B2 | 1120 | Mesotrione | 75 | AMATA | 21 | 100 |
| 2 | 27 | B2 | 1120 | Mesotrione | 75 | AMATA | 21 | 100 |
| 2 | 27 | B2 | 1120 | Mesotrione | 75 | AMATA | 21 | 100 |
| 2 | 28 | B2 | 0 | Mesotrione | 150 | AMATA | 21 | 100 |
| 2 | 28 | B2 | 0 | Mesotrione | 150 | AMATA | 21 | 100 |
| 2 | 28 | B2 | 0 | Mesotrione | 150 | AMATA | 21 | 100 |
| 2 | 29 | B2 | 560 | Mesotrione | 150 | AMATA | 21 | 100 |
| 2 | 29 | B2 | 560 | Mesotrione | 150 | AMATA | 21 | 100 |
| 2 | 29 | B2 | 560 | Mesotrione | 150 | AMATA | 21 | 100 |
| 2 | 30 | B2 | 1120 | Mesotrione | 150 | AMATA | 21 | 100 |
| 2 | 30 | B2 | 1120 | Mesotrione | 150 | AMATA | 21 | 100 |
| 2 | 30 | B2 | 1120 | Mesotrione | 150 | AMATA | 21 | 100 |
| 2 | 31 | Untreated | | | | AMATA | 21 | 0 |
| 2 | 31 | Untreated | | | | AMATA | 21 | 0 |
| 2 | 31 | Untreated | | | | AMATA | 21 | 0 |
| 2 | 1 | Solv. Ck | 0 | none | 0 | CHEAL | 21 | 0 |
| | | untreated | | | | | | |
| 2 | 1 | Solv. Ck | 0 | none | 0 | CHEAL | 21 | 0 |
| | | untreated | | | | | | |
| 2 | 1 | Solv. Ck | 0 | none | 0 | CHEAL | 21 | 0 |
| | | untreated | | | | | | |
| 2 | 2 | B2 | 560 | none | | CHEAL | 21 | 98 |
| 2 | 2 | B2 | 560 | none | | CHEAL | 21 | 99 |
| 2 | 2 | B2 | 560 | none | | CHEAL | 21 | 100 |
| 2 | 3 | B2 | 1120 | none | | CHEAL | 21 | 100 |
| 2 | 3 | B2 | 1120 | none | | CHEAL | 21 | 100 |
| 2 | 3 | B2 | 1120 | none | | CHEAL | 21 | 100 |
| 2 | 4 | B2 | 0 | Glufosinate | 200 | CHEAL | 21 | 10 |
| 2 | 4 | B2 | 0 | Glufosinate | 200 | CHEAL | 21 | 15 |
| 2 | 4 | B2 | 0 | Glufosinate | 200 | CHEAL | 21 | 5 |
| 2 | 5 | B2 | 560 | Glufosinate | 200 | CHEAL | 21 | 92 |
| 2 | 5 | B2 | 560 | Glufosinate | 200 | CHEAL | 21 | 90 |
| 2 | 5 | B2 | 560 | Glufosinate | 200 | CHEAL | 21 | 95 |
| 2 | 6 | B2 | 1120 | Glufosinate | 200 | CHEAL | 21 | 95 |
| 2 | 6 | B2 | 1120 | Glufosinate | 200 | CHEAL | 21 | 95 |
| 2 | 6 | B2 | 1120 | Glufosinate | 200 | CHEAL | 21 | 98 |
| 2 | 7 | B2 | 0 | Glufosinate | 600 | CHEAL | 21 | 50 |
| 2 | 7 | B2 | 0 | Glufosinate | 600 | CHEAL | 21 | 40 |
| 2 | 7 | B2 | 0 | Glufosinate | 600 | CHEAL | 21 | 100 |
| 2 | 8 | B2 | 560 | Glufosinate | 600 | CHEAL | 21 | 98 |
| 2 | 8 | B2 | 560 | Glufosinate | 600 | CHEAL | 21 | 98 |
| 2 | 8 | B2 | 560 | Glufosinate | 600 | CHEAL | 21 | 100 |
| 2 | 9 | B2 | 1120 | Glufosinate | 600 | CHEAL | 21 | 95 |
| 2 | 9 | B2 | 1120 | Glufosinate | 600 | CHEAL | 21 | 95 |
| 2 | 9 | B2 | 1120 | Glufosinate | 600 | CHEAL | 21 | 100 |
| 2 | 10 | B2 | 0 | Glufosinate | 900 | CHEAL | 21 | 100 |
| 2 | 10 | B2 | 0 | Glufosinate | 900 | CHEAL | 21 | 50 |
| 2 | 10 | B2 | 0 | Glufosinate | 900 | CHEAL | 21 | 100 |
| 2 | 11 | B2 | 560 | Glufosinate | 900 | CHEAL | 21 | 90 |
| 2 | 11 | B2 | 560 | Glufosinate | 900 | CHEAL | 21 | 75 |
| 2 | 11 | B2 | 560 | Glufosinate | 900 | CHEAL | 21 | 100 |
| 2 | 12 | B2 | 1120 | Glufosinate | 900 | CHEAL | 21 | 100 |
| 2 | 12 | B2 | 1120 | Glufosinate | 900 | CHEAL | 21 | 100 |
| 2 | 12 | B2 | 1120 | Glufosinate | 900 | CHEAL | 21 | 100 |
| 2 | 13 | B2 | 0 | Diclosulam | 25 | CHEAL | 21 | 40 |
| 2 | 13 | B2 | 0 | Diclosulam | 25 | CHEAL | 21 | 30 |
| 2 | 13 | B2 | 0 | Diclosulam | 25 | CHEAL | 21 | 50 |
| 2 | 14 | B2 | 560 | Diclosulam | 25 | CHEAL | 21 | 98 |
| 2 | 14 | B2 | 560 | Diclosulam | 25 | CHEAL | 21 | 95 |
| 2 | 14 | B2 | 560 | Diclosulam | 25 | CHEAL | 21 | 90 |
| 2 | 15 | B2 | 1120 | Diclosulam | 25 | CHEAL | 21 | 98 |
| 2 | 15 | B2 | 1120 | Diclosulam | 25 | CHEAL | 21 | 100 |
| 2 | 15 | B2 | 1120 | Diclosulam | 25 | CHEAL | 21 | 97 |
| 2 | 16 | B2 | 0 | Diclosulam | 35 | CHEAL | 21 | 45 |
| 2 | 16 | B2 | 0 | Diclosulam | 35 | CHEAL | 21 | 35 |
| 2 | 16 | B2 | 0 | Diclosulam | 35 | CHEAL | 21 | 85 |
| 2 | 17 | B2 | 560 | Diclosulam | 35 | CHEAL | 21 | 98 |
| 2 | 17 | B2 | 560 | Diclosulam | 35 | CHEAL | 21 | 100 |
| 2 | 17 | B2 | 560 | Diclosulam | 35 | CHEAL | 21 | 85 |
| 2 | 18 | B2 | 1120 | Diclosulam | 35 | CHEAL | 21 | 100 |
| 2 | 18 | B2 | 1120 | Diclosulam | 35 | CHEAL | 21 | 99 |
| 2 | 18 | B2 | 1120 | Diclosulam | 35 | CHEAL | 21 | 100 |
| 2 | 19 | B2 | 0 | Arylex | 5 | CHEAL | 21 | 100 |
| 2 | 19 | B2 | 0 | Arylex | 5 | CHEAL | 21 | 100 |
| 2 | 19 | B2 | 0 | Arylex | 5 | CHEAL | 21 | 100 |
| 2 | 20 | B2 | 560 | Arvlex | 5 | CHEAL | 21 | 100 |
| 2 | 20 | B2 | 560 | Arvlex | 5 | CHEAL | 21 | 100 |
| 2 | 20 | B2 | 560 | Arylex | 5 | CHEAL | 21 | 100 |
| 2 | 21 | B2 | 1120 | Arylex | 5 | CHEAL | 21 | 100 |
| 2 | 21 | B2 | 1120 | Arylex | 5 | CHEAL | 21 | 100 |
| 2 | 21 | B2 | 1120 | Arylex | 5 | CHEAL | 21 | 99 |
| 2 | 22 | B2 | 0 | Arylex | 6.5 | CHEAL | 21 | 100 |
| 2 | 22 | B2 | 0 | Arylex | 6.5 | CHEAL | 21 | 100 |
| 2 | 22 | B2 | 0 | Arvlex | 6.5 | CHEAL | 21 | 100 |
| 2 | 23 | B2 | 560 | Arylex | 6.5 | CHEAL | 21 | 100 |
| 2 | 23 | B2 | 560 | Arylex | 6.5 | CHEAL | 21 | 100 |
| 2 | 23 | B2 | 560 | Arylex | 6.5 | CHEAL | 21 | 100 |
| 2 | 24 | B2 | 1120 | Arylex | 6.5 | CHEAL | 21 | 100 |
| 2 | 24 | B2 | 1120 | Arylex | 6.5 | CHEAL | 21 | 100 |
| 2 | 24 | B2 | 1120 | Arvlex | 6.5 | CHEAL | 21 | 100 |
| 2 | 25 | B2 | 0 | Mesotrione | 75 | CHEAL | 21 | 100 |
| 2 | 25 | B2 | 0 | Mesotrione | 75 | CHEAL | 21 | 100 |
| 2 | 25 | B2 | 0 | Mesotrione | 75 | CHEAL | 21 | 100 |
| 2 | 26 | B2 | 560 | Mesotrione | 75 | CHEAL | 21 | 100 |
| 2 | 26 | B2 | 560 | Mesotrione | 75 | CHEAL | 21 | 100 |
| 2 | 26 | B2 | 560 | Mesotrione | 75 | CHEAL | 21 | 100 |
| 2 | 27 | B2 | 1120 | Mesotrione | 75 | CHEAL | 21 | 100 |
| 2 | 27 | B2 | 1120 | Mesotrione | 75 | CHEAL | 21 | 100 |
| 2 | 27 | B2 | 1120 | Mesotrione | 75 | CHEAL | 21 | 100 |
| 2 | 28 | B2 | 0 | Mesotrione | 150 | CHEAL | 21 | 100 |
| 2 | 28 | B2 | 0 | Mesotrione | 150 | CHEAL | 21 | 100 |
| 2 | 28 | B2 | 0 | Mesotrione | 150 | CHEAL | 21 | 100 |
| 2 | 29 | B2 | 560 | Mesotrione | 150 | CHEAL | 21 | 100 |
| 2 | 29 | B2 | 560 | Mesotrione | 150 | CHEAL | 21 | 100 |
| 2 | 29 | B2 | 560 | Mesotrione | 150 | CHEAL | 21 | 100 |
| 2 | 30 | B2 | 1120 | Mesotrione | 150 | CHEAL | 21 | 100 |
| 2 | 30 | B2 | 1120 | Mesotrione | 150 | CHEAL | 21 | 100 |
| 2 | 30 | B2 | 1120 | Mesotrione | 150 | CHEAL | 21 | 100 |
| 2 | 31 | Untreated | | | | CHEAL | 21 | 0 |
| 2 | 31 | Untreated | | | | CHEAL | 21 | 0 |
| 2 | 31 | Untreated | | | | CHEAL | 21 | 0 |
| 2 | 1 | Solv. Ck | 0 | none | 0 | ERICA | 21 | 0 |
| | | untreated | | | | | | |
| 2 | 1 | Solv. Ck | 0 | none | 0 | ERICA | 21 | 0 |
| | | untreated | | | | | | |
| 2 | 1 | Solv. Ck | 0 | none | 0 | ERICA | 21 | 0 |
| | | untreated | | | | | | |
| 2 | 2 | B2 | 560 | none | | ERICA | 21 | 95 |
| 2 | 2 | B2 | 560 | none | | ERICA | 21 | 98 |
| 2 | 2 | B2 | 560 | none | | ERICA | 21 | 92 |
| 2 | 3 | B2 | 1120 | none | | ERICA | 21 | 98 |
| 2 | 3 | B2 | 1120 | none | | ERICA | 21 | 97 |
| 2 | 3 | B2 | 1120 | none | | ERICA | 21 | 98 |
| 2 | 4 | B2 | 0 | Glufosinate | 200 | ERICA | 21 | 100 |
| 2 | 4 | B2 | 0 | Glufosinate | 200 | ERICA | 21 | 100 |
| 2 | 4 | B2 | 0 | Glufosinate | 200 | ERICA | 21 | 100 |
| 2 | 5 | B2 | 560 | Glufosinate | 200 | ERICA | 21 | 100 |
| 2 | 5 | B2 | 560 | Glufosinate | 200 | ERICA | 21 | 100 |
| 2 | 5 | B2 | 560 | Glufosinate | 200 | ERICA | 21 | 100 |
| 2 | 6 | B2 | 1120 | Glufosinate | 200 | ERICA | 21 | 100 |
| 2 | 6 | B2 | 1120 | Glufosinate | 200 | ERICA | 21 | 100 |
| 2 | 6 | B2 | 1120 | Glufosinate | 200 | ERICA | 21 | 100 |
| 2 | 7 | B2 | 0 | Glufosinate | 600 | ERICA | 21 | 100 |
| 2 | 7 | B2 | 0 | Glufosinate | 600 | ERICA | 21 | 100 |
| 2 | 7 | B2 | 0 | Glufosinate | 600 | ERICA | 21 | 100 |
| 2 | 8 | B2 | 560 | Glufosinate | 600 | ERICA | 21 | 100 |
| 2 | 8 | B2 | 560 | Glufosinate | 600 | ERICA | 21 | 100 |
| 2 | 8 | B2 | 560 | Glufosinate | 600 | ERICA | 21 | 100 |
| 2 | 9 | B2 | 1120 | Glufosinate | 600 | ERICA | 21 | 100 |
| 2 | 9 | B2 | 1120 | Glufosinate | 600 | ERICA | 21 | 100 |
| 2 | 9 | B2 | 1120 | Glufosinate | 600 | ERICA | 21 | 100 |
| 2 | 10 | B2 | 0 | Glufosinate | 900 | ERICA | 21 | 100 |
| 2 | 10 | B2 | 0 | Glufosinate | 900 | ERICA | 21 | 100 |
| 2 | 10 | B2 | 0 | Glufosinate | 900 | ERICA | 21 | 100 |
| 2 | 11 | B2 | 560 | Glufosinate | 900 | ERICA | 21 | 100 |
| 2 | 11 | B2 | 560 | Glufosinate | 900 | ERICA | 21 | 100 |
| 2 | 11 | B2 | 560 | Glufosinate | 900 | ERICA | 21 | 100 |
| 2 | 12 | B2 | 1120 | Glufosinate | 900 | ERICA | 21 | 100 |
| 2 | 12 | B2 | 1120 | Glufosinate | 900 | ERICA | 21 | 100 |
| 2 | 12 | B2 | 1120 | Glufosinate | 900 | ERICA | 21 | 100 |
| 2 | 13 | B2 | 0 | Diclosulam | 25 | ERICA | 21 | 100 |
| 2 | 13 | B2 | 0 | Diclosulam | 25 | ERICA | 21 | 100 |
| 2 | 13 | B2 | 0 | Diclosulam | 25 | ERICA | 21 | 100 |
| 2 | 14 | B2 | 560 | Diclosulam | 25 | ERICA | 21 | 99 |
| 2 | 14 | B2 | 560 | Diclosulam | 25 | ERICA | 21 | 100 |
| 2 | 14 | B2 | 560 | Diclosulam | 25 | ERICA | 21 | 99 |
| 2 | 15 | B2 | 1120 | Diclosulam | 25 | ERICA | 21 | 98 |
| 2 | 15 | B2 | 1120 | Diclosulam | 25 | ERICA | 21 | 99 |
| 2 | 15 | B2 | 1120 | Diclosulam | 25 | ERICA | 21 | 100 |
| 2 | 16 | B2 | 0 | Diclosulam | 35 | ERICA | 21 | 100 |
| 2 | 16 | B2 | 0 | Diclosulam | 35 | ERICA | 21 | 100 |
| 2 | 16 | B2 | 0 | Diclosulam | 35 | ERICA | 21 | 100 |
| 2 | 17 | B2 | 560 | Diclosulam | 35 | ERICA | 21 | 100 |
| 2 | 17 | B2 | 560 | Diclosulam | 35 | ERICA | 21 | 100 |
| 2 | 17 | B2 | 560 | Diclosulam | 35 | ERICA | 21 | 100 |
| 2 | 18 | B2 | 1120 | Diclosulam | 35 | ERICA | 21 | 100 |
| 2 | 18 | B2 | 1120 | Diclosulam | 35 | ERICA | 21 | 100 |
| 2 | 18 | B2 | 1120 | Diclosulam | 35 | ERICA | 21 | 100 |
| 2 | 19 | B2 | 0 | Arvlex | 5 | ERICA | 21 | 95 |
| 2 | 19 | B2 | 0 | Arylex | 5 | ERICA | 21 | 95 |
| 2 | 19 | B2 | 0 | Arylex | 5 | ERICA | 21 | 95 |
| 2 | 20 | B2 | 560 | Arylex | 5 | ERICA | 21 | 98 |
| 2 | 20 | B2 | 560 | Arylex | 5 | ERICA | 21 | 98 |
| 2 | 20 | B2 | 560 | Arylex | 5 | ERICA | 21 | 98 |
| 2 | 21 | B2 | 1120 | Aryley | 5 | ERICA | 21 | 98 |
| 2 | 21 | B2 | 1120 | Arylex | 5 | ERICA | 21 | 99 |
| 2 | 21 | B2 | 1120 | Arylex | 5 | ERICA | 21 | 99 |
| 2 | 22 | B2 | 0 | Arylex | 6.5 | ERICA | 21 | 99 |
| 2 | 22 | B2 | 0 | Arylex | 6.5 | ERICA | 21 | 95 |
| 2 | 22 | B2 | 0 | Arylex | 6.5 | ERICA | 21 | 97 |
| 2 | 23 | B2 | 560 | Arylex | 6.5 | ERICA | 21 | 95 |
| 2 | 23 | B2 | 560 | Arylex | 6.5 | ERICA | 21 | 95 |
| 2 | 23 | B2 | 560 | Arylex | 6.5 | ERICA | 21 | 98 |
| 2 | 24 | B2 | 1120 | Arylex | 6.5 | ERICA | 21 | 95 |
| 2 | 24 | B2 | 1120 | Arylex | 6.5 | ERICA | 21 | 95 |
| 2 | 24 | B2 | 1120 | Arylex | 6.5 | ERICA | 21 | 97 |
| 2 | 25 | B2 | 0 | Mesotrone | 75 | ERICA | 21 | 92 |
| 2 | 25 | B2 | 0 | Mesotrione | 75 | ERICA | 21 | 92 |
| 2 | 25 | B2 | 0 | Mesotrione | 75 | ERICA | 21 | 92 |
| 2 | 26 | B2 | 560 | Mesotrione | 75 | ERICA | 21 | 100 |
| 2 | 26 | B2 | 560 | Mesotrione | 75 | ERICA | 21 | 100 |
| 2 | 26 | B2 | 560 | Mesotrione | 75 | ERICA | 21 | 100 |
| 2 | 27 | B2 | 1120 | Mesotrione | 75 | ERICA | 21 | 100 |
| 2 | 27 | B2 | 1120 | Mesotrione | 75 | ERICA | 21 | 100 |
| 2 | 27 | B2 | 1120 | Mesotrione | 75 | ERICA | 21 | 100 |
| 2 | 28 | B2 | 0 | Mesotrione | 150 | ERICA | 21 | 95 |
| 2 | 28 | B2 | 0 | Mesotrione | 150 | ERICA | 21 | 100 |
| 2 | 28 | B2 | 0 | Mesotrione | 150 | ERICA | 21 | 100 |
| 2 | 29 | B2 | 560 | Mesotrione | 150 | ERICA | 21 | 100 |
| 2 | 29 | B2 | 560 | Mesotrione | 150 | ERICA | 21 | 100 |
| 2 | 20 | B2 | 560 | Mesotrione | 150 | ERICA | 21 | 100 |
| 2 | 30 | B2 | 1120 | Mesotrione | 150 | ERICA | 21 | 100 |
| 2 | 30 | B2 | 1120 | Mesotrione | 150 | ERICA | 21 | 100 |
| 2 | 30 | B2 | 1120 | Mesotrione | 150 | ERICA | 21 | 100 |
| 2 | 31 | Untreated | | | | ERICA | 21 | 0 |
| 2 | 31 | Untreated | | | | ERICA | 21 | 0 |
| 2 | 31 | Untreated | | | | ERICA | 21 | 0 |
| 2 | 1 | Solv. Ck | 0 | none | 0 | ZEAMX | 21 | 0 |
| | | untreated | | | | | | |
| 2 | 1 | Solv. Ck | 0 | none | 0 | ZEAMX | 21 | 0 |
| | | untreated | | | | | | |
| 2 | 1 | Solv. Ck | 0 | none | 0 | ZEAMX | 21 | 0 |
| | | untreated | | | | | | |
| 2 | 2 | B2 | 560 | none | | ZEAMX | 21 | 3 |
| 2 | 2 | B2 | 560 | none | | ZEAMX | 21 | 3 |
| 2 | 2 | B2 | 560 | none | | ZEAMX | 21 | 5 |
| 2 | 3 | B2 | 1120 | none | | ZEAMX | 21 | 5 |
| 2 | 3 | B2 | 1120 | none | | ZEAMX | 21 | 5 |
| 2 | 3 | B2 | 1120 | none | | ZEAMX | 21 | 3 |
| 2 | 4 | B2 | 0 | Glufosinate | 200 | ZEAMX | 21 | 5 |
| 2 | 4 | B2 | 0 | Glufosinate | 200 | ZEAMX | 21 | 10 |
| 2 | 4 | B2 | 0 | Glufosinate | 200 | ZEAMX | 21 | 5 |
| 2 | 5 | B2 | 560 | Glufosinate | 200 | ZEAMX | 21 | 5 |
| 2 | 5 | B2 | 560 | Glufosinate | 200 | ZEAMX | 21 | 5 |
| 2 | 5 | B2 | 560 | Glufosinate | 200 | ZEAMX | 21 | 5 |
| 2 | 6 | B2 | 1120 | Glufosinate | 200 | ZEAMX | 21 | 5 |
| 2 | 6 | B2 | 1120 | Glufosinate | 200 | ZEAMX | 21 | 10 |
| 2 | 6 | B2 | 1120 | Glufosinate | 200 | ZEAMX | 21 | 10 |
| 2 | 7 | B2 | 0 | Glufosinate | 600 | ZEAMX | 21 | 0 |
| 2 | 7 | B2 | 0 | Glufosinate | 600 | ZEAMX | 21 | 5 |
| 2 | 7 | B2 | 0 | Glufosinate | 600 | ZEAMX | 21 | 10 |
| 2 | 8 | B2 | 560 | Glufosinate | 600 | ZEAMX | 21 | 10 |
| 2 | 8 | B2 | 560 | Glufosinate | 600 | ZEAMX | 21 | 10 |
| 2 | 8 | B2 | 560 | Glufosinate | 600 | ZEAMX | 21 | 10 |
| 2 | 9 | B2 | 1120 | Glufosinate | 600 | ZEAMX | 21 | 15 |
| 2 | 9 | B2 | 1120 | Glufosinate | 600 | ZEAMX | 21 | 15 |
| 2 | 9 | B2 | 1120 | Glufosinate | 600 | ZEAMX | 21 | 10 |
| 2 | 10 | B2 | 0 | Glufosinate | 900 | ZEAMX | 21 | 10 |
| 2 | 10 | B2 | 0 | Glufosinate | 900 | ZEAMX | 21 | 10 |
| 2 | 10 | B2 | 0 | Glufosinate | 900 | ZEAMX | 21 | 20 |
| 2 | 11 | B2 | 560 | Glufosinate | 900 | ZEAMX | 21 | 35 |
| 2 | 11 | B2 | 560 | Glufosinate | 900 | ZEAMX | 21 | 35 |
| 2 | 11 | B2 | 560 | Glufosinate | 900 | ZEAMX | 21 | 65 |
| 2 | 12 | B2 | 1120 | Glufosinate | 900 | ZEAMX | 21 | 15 |
| 2 | 12 | B2 | 1120 | Glufosinate | 900 | ZEAMX | 21 | 65 |
| 2 | 12 | B2 | 1120 | Glufosinate | 900 | ZEAMX | 21 | 70 |
| 2 | 13 | B2 | 0 | Diclosulam | 25 | ZEAMX | 21 | 80 |
| 2 | 13 | B2 | 0 | Diclosulam | 25 | ZEAMX | 21 | 85 |
| 2 | 13 | B2 | 0 | Diclosulam | 25 | ZEAMX | 21 | 80 |
| 2 | 14 | B2 | 560 | Diclosulam | 25 | ZEAMX | 21 | 50 |
| 2 | 14 | B2 | 560 | Diclosulam | 25 | ZEAMX | 21 | 65 |
| 2 | 14 | B2 | 560 | Diclosulam | 25 | ZEAMX | 21 | 65 |
| 2 | 15 | B2 | 1120 | Diclosulam | 25 | ZEAMX | 21 | 50 |
| 2 | 15 | B2 | 1120 | Diclosulam | 25 | ZEAMX | 21 | 75 |
| 2 | 15 | B2 | 1120 | Diclosulam | 25 | ZEAMX | 21 | 55 |
| 2 | 16 | B2 | 0 | Diclosulam | 35 | ZEAMX | 21 | 95 |
| 2 | 16 | B2 | 0 | Diclosulam | 35 | ZEAMX | 21 | 90 |
| 2 | 16 | B2 | 0 | Diclosulam | 35 | ZEAMX | 21 | 85 |
| 2 | 17 | B2 | 560 | Diclosulam | 35 | ZEAMX | 21 | 75 |
| 2 | 17 | B2 | 560 | Diclosulam | 35 | ZEAMX | 21 | 80 |
| 2 | 17 | B2 | 560 | Diclosulam | 35 | ZEAMX | 21 | 80 |
| 2 | 18 | B2 | 1120 | Diclosulam | 35 | ZEAMX | 21 | 70 |
| 2 | 18 | B2 | 1120 | Diclosulam | 35 | ZEAMX | 21 | 65 |
| 2 | 18 | B2 | 1120 | Diclosulam | 35 | ZEAMX | 21 | 70 |
| 2 | 19 | B2 | 0 | Arylex | 5 | ZEAMX | 21 | 70 |
| 2 | 19 | B2 | 0 | Aryles | 5 | ZEAMX | 21 | 65 |
| 2 | 19 | B2 | 0 | Arylex | 5 | ZEAMX | 21 | 65 |
| 2 | 20 | B2 | 560 | Arylex | 5 | ZEAMX | 21 | 5 |
| 2 | 20 | B2 | 560 | Arylex | 5 | ZEAMX | 21 | 5 |
| 2 | 20 | B2 | 560 | Arylex | 5 | ZEAMX | 21 | 5 |
| 2 | 21 | B2 | 1120 | Arylex | 5 | ZEAMX | 21 | 5 |
| 2 | 21 | B2 | 1120 | Arvlex | 5 | ZEAMX | 21 | 10 |
| 2 | 21 | B2 | 1120 | Arylex | 5 | ZEAMX | 21 | 15 |
| 2 | 22 | B2 | 0 | Arylex | 6.5 | ZEAMX | 21 | 75 |
| 2 | 22 | B2 | 0 | Arylex | 6.5 | ZEAMX | 21 | 70 |
| 2 | 22 | B2 | 0 | Arylex | 6.5 | ZEAMX | 21 | 65 |
| 2 | 23 | B2 | 560 | Arylex | 6.5 | ZEAMX | 21 | 3 |
| 2 | 23 | B2 | 560 | Arylex | 6.5 | ZEAMX | 21 | 15 |
| 2 | 23 | B2 | 560 | Arylex | 6.5 | ZEAMX | 21 | 10 |
| 2 | 24 | B2 | 1120 | Arylex | 6.5 | ZEAMX | 21 | 15 |
| 2 | 24 | B2 | 1120 | Arylex | 6.5 | ZEAMX | 21 | 5 |
| 2 | 24 | B2 | 1120 | Arylex | 6.5 | ZEAMX | 21 | 10 |
| 2 | 25 | B2 | 0 | Mesotrione | 75 | ZEAMX | 21 | 3 |
| 2 | 25 | B2 | 0 | Mesotrione | 75 | ZEAMX | 21 | 0 |
| 2 | 25 | B2 | 0 | Mesotrione | 75 | ZEAMX | 21 | 0 |
| 2 | 26 | B2 | 560 | Mesotrione | 75 | ZEAMX | 21 | 3 |
| 2 | 26 | B2 | 560 | Mesotrione | 75 | ZEAMX | 21 | 3 |
| 2 | 26 | B2 | 560 | Mesotrione | 75 | ZEAMX | 21 | 5 |
| 2 | 27 | B2 | 1120 | Mesotrione | 75 | ZEAMX | 21 | 3 |
| 2 | 27 | B2 | 1120 | Mesotrione | 75 | ZEAMX | 21 | 2 |
| 2 | 27 | B2 | 1120 | Mesotrione | 75 | ZEAMX | 21 | 5 |
| 2 | 28 | B2 | 0 | Mesotrione | 150 | ZEAMX | 21 | 0 |
| 2 | 28 | B2 | 0 | Mesotrione | 150 | ZEAMX | 21 | 5 |
| 2 | 28 | B2 | 0 | Mesotrione | 150 | ZEAMX | 21 | 0 |
| 2 | 29 | B2 | 560 | Mesotrione | 150 | ZEAMX | 21 | 10 |
| 2 | 29 | B2 | 560 | Mesotrione | 150 | ZEAMX | 21 | 10 |
| 2 | 29 | B2 | 560 | Mesotrione | 150 | ZEAMX | 21 | 5 |
| 2 | 30 | B2 | 1120 | Mesotrione | 150 | ZEAMX | 21 | 5 |
| 2 | 30 | B2 | 1120 | Mesotrione | 150 | ZEAMX | 21 | 5 |
| 2 | 30 | B2 | 1120 | Mesotrione | 150 | ZEAMX | 21 | 5 |
| 2 | 31 | Untreated | | | | ZEAMX | 21 | 0 |
| 2 | 31 | Untreated | | | | ZEAMX | 21 | 0 |
| 2 | 31 | Untreated | | | | ZEAMX | 21 | 0 |
| Bridge 1 | 1 | Solv. Ck | 0 | 0 | 0 | ABUTH | 21 | 0 |
| | | untreated | | | | | | |
| Bridge 1 | 1 | Solv. Ck | 0 | 0 | 0 | ABUTH | 21 | 0 |
| | | untreated | | | | | | |
| Bridge 1 | 1 | Solv. Ck | 0 | 0 | 0 | ABUTH | 21 | 0 |
| | | untreated | | | | | | |
| Bridge 1 | 2 | D10 | 560 | none | 0 | ABUTH | 21 | 99 |
| Bridge 1 | 2 | D10 | 560 | none | 0 | ABUTH | 21 | 100 |
| Bridge 1 | 2 | D10 | 560 | none | 0 | ABUTH | 21 | 100 |
| Bridge 1 | 3 | D10 | 1120 | none | 0 | ABUTH | 21 | 100 |
| Bridge 1 | 3 | D10 | 1120 | none | 0 | ABUTH | 21 | 100 |
| Bridge 1 | 3 | D10 | 1120 | none | 0 | ABUTH | 21 | 100 |
| Bridge 1 | 4 | D10 | 0 | diclosulam | 35 | ABUTH | 21 | 100 |
| Bridge 1 | 4 | D10 | 0 | diclosulam | 35 | ABUTH | 21 | 100 |
| Bridge 1 | 4 | D10 | 0 | diclosulam | 35 | ABUTH | 21 | 100 |
| Bridge 1 | 5 | D10 | 560 | diclosulam | 35 | ABUTH | 21 | 100 |
| Bridge 1 | 5 | D10 | 560 | diclosulam | 35 | ABUTH | 21 | 100 |
| Bridge 1 | 5 | D10 | 560 | diclosulam | 35 | ABUTH | 21 | 100 |
| Bridge 1 | 6 | D10 | 1120 | diclosulam | 35 | ABUTH | 21 | 100 |
| Bridge 1 | 6 | D10 | 1120 | diclosulam | 35 | ABUTH | 21 | 100 |
| Bridge 1 | 6 | D10 | 1120 | diclosulam | 35 | ABUTH | 21 | 100 |
| Bridge 1 | 7 | D10 | 0 | Arvlex | 5 | ABUTH | 21 | 100 |
| Bridge 1 | 7 | D10 | 0 | Arylex | 5 | ABUTH | 21 | 100 |
| Bridge 1 | 7 | D10 | 0 | Arylex | 5 | ABUTH | 21 | 100 |
| Bridge 1 | 8 | D10 | 560 | Arylex | 5 | ABUTH | 21 | 100 |
| Bridge 1 | 8 | D10 | 560 | Arvlex | 5 | ABUTH | 21 | 100 |
| Bridge 1 | 8 | D10 | 560 | Arylex | 5 | ABUTH | 21 | 100 |
| Bridge 1 | 9 | D10 | 1120 | Arvlex | 5 | ABUTH | 21 | 100 |
| Bridge 1 | 9 | D10 | 1120 | Arylex | 5 | ABUTH | 21 | 100 |
| Bridge 1 | 9 | D10 | 1120 | Arylex | 5 | ABUTH | 21 | 100 |
| Bridge 1 | 10 | D10 | 0 | Mesotrione | 150 | ABUTH | 21 | 100 |
| Bridge 1 | 10 | D10 | 0 | Mesotrione | 150 | ABUTH | 21 | 100 |
| Bridge 1 | 10 | D10 | 0 | Mesotrione | 150 | ABUTH | 21 | 100 |
| Bridge 1 | 11 | D10 | 560 | Mesotrione | 150 | ABUTH | 21 | 100 |
| Bridge 1 | 11 | D10 | 560 | Mesotrione | 150 | ABUTH | 21 | 100 |
| Bridge 1 | 11 | D10 | 560 | Mesotrione | 150 | ABUTH | 21 | 100 |
| Bridge 1 | 12 | D10 | 1120 | Mesotrione | 150 | ABUTH | 21 | 100 |
| Bridge 1 | 12 | D10 | 1120 | Mesotrione | 150 | ABUTH | 21 | 100 |
| Bridge 1 | 12 | D10 | 1120 | Mesotrione | 150 | ABUTH | 21 | 100 |
| Bridge 1 | 13 | D10 | 0 | Glyphosate | 1120 | ABUTH | 21 | 95 |
| Bridge 1 | 13 | D10 | 0 | Glyphosate | 1120 | ABUTH | 21 | 97 |
| Bridge 1 | 13 | D10 | 0 | Glyphosate | 1120 | ABUTH | 21 | 100 |
| Bridge 1 | 14 | D10 | 560 | Glyphosate | 1120 | ABUTH | 21 | 100 |
| Bridge 1 | 14 | D10 | 560 | Glyphosate | 1120 | ABUTH | 21 | 100 |
| Bridge 1 | 14 | D10 | 560 | Glyphosate | 1120 | ABUTH | 21 | 100 |
| Bridge 1 | 15 | D10 | 1120 | Glyphosate | 1120 | ABUTH | 21 | 100 |
| Bridge 1 | 15 | D10 | 1120 | Glyphosate | 1120 | ABUTH | 21 | 100 |
| Bridge 1 | 15 | D10 | 1120 | Glyphosate | 1120 | ABUTH | 21 | 100 |
| Bridge 1 | 16 | D10 | 0 | Glufosinate | 600 | ABUTH | 21 | 100 |
| Bridge 1 | 16 | D10 | 0 | Glufosinate | 600 | ABUTH | 21 | 99 |
| Bridge 1 | 16 | D10 | 0 | Glufosinate | 600 | ABUTH | 21 | 99 |
| Bridge 1 | 17 | D10 | 560 | Glufosinate | 600 | ABUTH | 21 | 100 |
| Bridge 1 | 17 | D10 | 560 | Glufosinate | 600 | ABUTH | 21 | 100 |
| Bridge 1 | 17 | D10 | 560 | Glufosinate | 600 | ABUTH | 21 | 100 |
| Bridge 1 | 18 | D10 | 1120 | Glufosinate | 600 | ABUTH | 21 | 100 |
| Bridge 1 | 18 | D10 | 1120 | Glufosinate | 600 | ABUTH | 21 | 99 |
| Bridge 1 | 18 | D10 | 1120 | Glufosinate | 600 | ABUTH | 21 | 99 |
| Bridge 1 | 19 | D8 | 560 | none | 0 | ABUTH | 21 | 100 |
| Bridge 1 | 19 | D8 | 560 | none | 0 | ABUTH | 21 | 100 |
| Bridge 1 | 19 | D8 | 560 | none | 0 | ABUTH | 21 | 100 |
| Bridge 1 | 20 | D8 | 1120 | none | 0 | ABUTH | 21 | 100 |
| Bridge 1 | 20 | D8 | 1120 | none | 0 | ABUTH | 21 | 100 |
| Bridge 1 | 20 | D8 | 1120 | none | 0 | ABUTH | 21 | 100 |
| Bridge 1 | 21 | D8 | 560 | diclosulam | 35 | ABUTH | 21 | 100 |
| Bridge 1 | 21 | D8 | 560 | diclosulam | 35 | ABUTH | 21 | 100 |
| Bridge 1 | 21 | D8 | 560 | diclosulam | 35 | ABUTH | 21 | 100 |
| Bridge 1 | 22 | D8 | 1120 | diclosulam | 35 | ABUTH | 21 | 100 |
| Bridge 1 | 22 | D8 | 1120 | diclosulam | 35 | ABUTH | 21 | 100 |
| Bridge 1 | 22 | D8 | 1120 | diclosulam | 35 | ABUTH | 21 | 100 |
| Bridge 1 | 23 | D8 | 560 | Arylex | 5 | ABUTH | 21 | 100 |
| Bridge 1 | 23 | D8 | 560 | Arylex | 5 | ABUTH | 21 | 100 |
| Bridge 1 | 23 | D8 | 560 | Arylex | 5 | ABUTH | 21 | 100 |
| Bridge 1 | 24 | D8 | 1120 | Arylex | 5 | ABUTH | 21 | 100 |
| Bridge 1 | 24 | D8 | 1120 | Arylex | 5 | ABUTH | 21 | 100 |
| Bridge 1 | 24 | D8 | 1120 | Arylex | 5 | ABUTH | 21 | 100 |
| Bridge 1 | 25 | D8 | 560 | Mesotrione | 150 | ABUTH | 21 | 100 |
| Bridge 1 | 25 | D8 | 560 | Mesotrione | 150 | ABUTH | 21 | 100 |
| Bridge 1 | 25 | D8 | 560 | Mesotrione | 150 | ABUTH | 21 | 100 |
| Bridge 1 | 26 | D8 | 1120 | Mesotrione | 150 | ABUTH | 21 | 100 |
| Bridge 1 | 26 | D8 | 1120 | Mesotrione | 150 | ABUTH | 21 | 100 |
| Bridge 1 | 26 | D8 | 1120 | Mesotrione | 150 | ABUTH | 21 | 100 |
| Bridge 1 | 27 | D8 | 560 | Glyphosate | 1120 | ABUTH | 21 | 100 |
| Bridge 1 | 27 | D8 | 560 | Glyphosate | 1120 | ABUTH | 21 | 100 |
| Bridge 1 | 27 | D8 | 560 | Glyphosate | 1120 | ABUTH | 21 | 100 |
| Bridge 1 | 28 | D8 | 1120 | Glyphosate | 1120 | ABUTH | 21 | 100 |
| Bridge 1 | 28 | D8 | 1120 | Glyphosate | 1120 | ABUTH | 21 | 100 |
| Bridge 1 | 28 | D8 | 1120 | Glyphosate | 1120 | ABUTH | 21 | 100 |
| Bridge 1 | 29 | D8 | 560 | Glufosinate | 600 | ABUTH | 21 | 100 |
| Bridge 1 | 29 | D8 | 560 | Glufosinate | 600 | ABUTH | 21 | 97 |
| Bridge 1 | 29 | D8 | 560 | Glufosinate | 600 | ABUTH | 21 | 100 |
| Bridge 1 | 30 | D8 | 1120 | Glufosinate | 600 | ABUTH | 21 | 100 |
| Bridge 1 | 30 | D8 | 1120 | Glufosinate | 600 | ABUTH | 21 | 100 |
| Bridge 1 | 30 | D8 | 1120 | Glufosinate | 600 | ABUTH | 21 | 100 |
| Bridge 1 | 31 | D27 | 560 | none | 0 | ABUTH | 21 | 100 |
| Bridge 1 | 31 | D27 | 560 | none | 0 | ABUTH | 21 | 100 |
| Bridge 1 | 31 | D27 | 560 | none | 0 | ABUTH | 21 | 100 |
| Bridge 1 | 32 | D27 | 1120 | none | 0 | ABUTH | 21 | 100 |
| Bridge 1 | 32 | D27 | 1120 | none | 0 | ABUTH | 21 | 100 |
| Bridge 1 | 32 | D27 | 1120 | none | 0 | ABUTH | 21 | 100 |
| Bridge 1 | 33 | D27 | 560 | diclosulam | 35 | ABUTH | 21 | 100 |
| Bridge 1 | 33 | D27 | 560 | diclosulam | 35 | ABUTH | 21 | 100 |
| Bridge 1 | 33 | D27 | 560 | diclosulam | 35 | ABUTH | 21 | 100 |
| Bridge 1 | 34 | D27 | 1120 | diclosulam | 35 | ABUTH | 21 | 100 |
| Bridge 1 | 34 | D27 | 1120 | diclosulam | 35 | ABUTH | 21 | 100 |
| Bridge 1 | 34 | D27 | 1120 | diclosulam | 35 | ABUTH | 21 | 100 |
| Bridge 1 | 35 | D27 | 560 | Arvlex | 5 | ABUTH | 21 | 100 |
| Bridge 1 | 35 | D27 | 560 | Arylex | 5 | ABUTH | 21 | 100 |
| Bridge 1 | 35 | D27 | 560 | Arylex | 5 | ABUTH | 21 | 100 |
| Bridge 1 | 36 | D27 | 1120 | Arylex | 5 | ABUTH | 21 | 100 |
| Bridge 1 | 36 | D27 | 1120 | Arylex | 5 | ABUTH | 21 | 100 |
| Bridge 1 | 36 | D27 | 1120 | Arylex | 5 | ABUTH | 21 | 100 |
| Bridge 1 | 37 | D27 | 560 | Mesotrione | 150 | ABUTH | 21 | 100 |
| Bridge 1 | 37 | D27 | 560 | Mesotrione | 150 | ABUTH | 21 | 100 |
| Bridge 1 | 37 | D27 | 560 | Mesotrione | 150 | ABUTH | 21 | 100 |
| Bridge 1 | 38 | D27 | 1120 | Mesotrione | 150 | ABUTH | 21 | 100 |
| Bridge 1 | 38 | D27 | 1120 | Mesotrione | 150 | ABUTH | 21 | 100 |
| Bridge 1 | 38 | D27 | 1120 | Mesotrione | 150 | ABUTH | 21 | 100 |
| Bridge 1 | 39 | D27 | 560 | Glyphosate | 1120 | ABUTH | 21 | 100 |
| Bridge 1 | 39 | D27 | 560 | Glyphosate | 1120 | ABUTH | 21 | 100 |
| Bridge 1 | 39 | D27 | 560 | Glyphosate | 1120 | ABUTH | 21 | 100 |
| Bridge 1 | 40 | D27 | 1120 | Glyphosate | 1120 | ABUTH | 21 | 100 |
| Bridge 1 | 40 | D27 | 1120 | Glyphosate | 1120 | ABUTH | 21 | 100 |
| Bridge 1 | 40 | D27 | 1120 | Glyphosate | 1120 | ABUTH | 21 | 100 |
| Bridge 1 | 41 | D27 | 560 | Glufosinate | 600 | ABUTH | 21 | 100 |
| Bridge 1 | 41 | D27 | 560 | Glufosinate | 600 | ABUTH | 21 | 100 |
| Bridge 1 | 41 | D27 | 560 | Glufosinate | 600 | ABUTH | 21 | 100 |
| Bridge 1 | 42 | D27 | 1120 | Glufosinate | 600 | ABUTH | 21 | 100 |
| Bridge 1 | 42 | D27 | 1120 | Glufosinate | 600 | ABUTH | 21 | 100 |
| Bridge 1 | 42 | D27 | 1120 | Glufosinate | 600 | ABUTH | 21 | 100 |
| Bridge 1 | 43 | B2 | 1120 | none | | ABUTH | 21 | 100 |
| Bridge 1 | 43 | B2 | 1120 | none | | ABUTH | 21 | 100 |
| Bridge 1 | 43 | B2 | 1120 | none | | ABUTH | 21 | 100 |
| Bridge 1 | 44 | B2 | 1120 | diclosulam | 35 | ABUTH | 21 | 100 |
| Bridge 1 | 44 | B2 | 1120 | diclosulam | 35 | ABUTH | 21 | 100 |
| Bridge 1 | 44 | B2 | 1120 | diclosulam | 35 | ABUTH | 21 | 100 |
| Bridge 1 | 45 | B2 | 1120 | Arylex | 5 | ABUTH | 21 | 100 |
| Bridge 1 | 45 | B2 | 1120 | Arylex | 5 | ABUTH | 21 | 100 |
| Bridge 1 | 45 | B2 | 1120 | Arylex | 5 | ABUTH | 21 | 100 |
| Bridge 1 | 46 | B2 | 1120 | Mesotrione | 150 | ABUTH | 21 | 100 |
| Bridge 1 | 46 | B2 | 1120 | Mesotrione | 150 | ABUTH | 21 | 100 |
| Bridge 1 | 46 | B2 | 1120 | Mesotrione | 150 | ABUTH | 21 | 100 |
| Bridge 1 | 47 | B2 | 1120 | Glyphosate | 1120 | ABUTH | 21 | 100 |
| Bridge 1 | 47 | B2 | 1120 | Glyphosate | 1120 | ABUTH | 21 | 100 |
| Bridge 1 | 47 | B2 | 1120 | Glyphosate | 1120 | ABUTH | 21 | 100 |
| Bridge 1 | 48 | B2 | 1120 | Glufosinate | 600 | ABUTH | 21 | 100 |
| Bridge 1 | 48 | B2 | 1120 | Glufosinate | 600 | ABUTH | 21 | 100 |
| Bridge 1 | 48 | B2 | 1120 | Glufosinate | 600 | ABUTH | 21 | 100 |
| Bridge 1 | 1 | Solv. Ck | 0 | 0 | 0 | AMATA | 21 | 0 |
| | | untreated | | | | | | |
| Bridge 1 | 1 | Solv. Ck | 0 | 0 | 0 | AMATA | 21 | 0 |
| | | untreated | | | | | | |
| Bridge 1 | 1 | Solv. Ck | 0 | 0 | 0 | AMATA | 21 | 0 |
| | | untreated | | | | | | |
| Bridge 1 | 2 | D10 | 560 | none | 0 | AMATA | 21 | 100 |
| Bridge 1 | 2 | D10 | 560 | none | 0 | AMATA | 21 | 100 |
| Bridge 1 | 2 | D10 | 560 | none | 0 | AMATA | 21 | 100 |
| Bridge 1 | 3 | D10 | 1120 | none | 0 | AMATA | 21 | 100 |
| Bridge 1 | 3 | D10 | 1120 | none | 0 | AMATA | 21 | 100 |
| Bridge 1 | 3 | D10 | 1120 | none | 0 | AMATA | 21 | 100 |
| Bridge 1 | 4 | D10 | 0 | diclosulam | 35 | AMATA | 21 | 10 |
| Bridge 1 | 4 | D10 | 0 | diclosulam | 35 | AMATA | 21 | 0 |
| Bridge 1 | 4 | D10 | 0 | diclosulam | 35 | AMATA | 21 | 10 |
| Bridge 1 | 5 | D10 | 560 | diclosulam | 35 | AMATA | 21 | 100 |
| Bridge 1 | 5 | D10 | 560 | diclosulam | 35 | AMATA | 21 | 100 |
| Bridge 1 | 5 | D10 | 560 | diclosulam | 35 | AMATA | 21 | 100 |
| Bridge 1 | 6 | D10 | 1120 | diclosulam | 35 | AMATA | 21 | 100 |
| Bridge 1 | 6 | D10 | 1120 | diclosulam | 35 | AMATA | 21 | 85 |
| Bridge 1 | 6 | D10 | 1120 | diclosulam | 35 | AMATA | 21 | 100 |
| Bridge 1 | 7 | D10 | 0 | Arylex | 5 | AMATA | 21 | 100 |
| Bridge 1 | 7 | D10 | 0 | Arylex | 5 | AMATA | 21 | 65 |
| Bridge 1 | 7 | D10 | 0 | Arylex | 5 | AMATA | 21 | 70 |
| Bridge 1 | 8 | D10 | 560 | Arylex | 5 | AMATA | 21 | 100 |
| Bridge 1 | 8 | D10 | 560 | Arvlex | 5 | AMATA | 21 | 80 |
| Bridge 1 | 8 | D10 | 560 | Arylex | 5 | AMATA | 21 | 100 |
| Bridge 1 | 9 | D10 | 1120 | Arylex | 5 | AMATA | 21 | 100 |
| Bridge 1 | 9 | D10 | 1120 | Arylex | 5 | AMATA | 21 | 100 |
| Bridge 1 | 9 | D10 | 1120 | Arylex | 5 | AMATA | 21 | 100 |
| Bridge 1 | 10 | D10 | 0 | Mesotrione | 150 | AMATA | 21 | 100 |
| Bridge 1 | 10 | D10 | 0 | Mesotrione | 150 | AMATA | 21 | 100 |
| Bridge 1 | 10 | D10 | 0 | Mesotrione | 150 | AMATA | 21 | 100 |
| Bridge 1 | 11 | D10 | 560 | Mesotrione | 150 | AMATA | 21 | 100 |
| Bridge 1 | 11 | D10 | 560 | Mesotrione | 150 | AMATA | 21 | 100 |
| Bridge 1 | 11 | D10 | 560 | Mesotrione | 150 | AMATA | 21 | 100 |
| Bridge 1 | 12 | D10 | 1120 | Mesotrione | 150 | AMATA | 21 | 100 |
| Bridge 1 | 12 | D10 | 1120 | Mesotrione | 150 | AMATA | 21 | 100 |
| Bridge 1 | 12 | D10 | 1120 | Mesotrione | 150 | AMATA | 21 | 100 |
| Bridge 1 | 13 | D10 | 0 | Glyphosate | 1120 | AMATA | 21 | 98 |
| Bridge 1 | 13 | D10 | 0 | Glyphosate | 1120 | AMATA | 21 | 100 |
| Bridge 1 | 13 | D10 | 0 | Glyphosate | 1120 | AMATA | 21 | 100 |
| Bridge 1 | 14 | D10 | 560 | Glyphosate | 1120 | AMATA | 21 | 100 |
| Bridge 1 | 14 | D10 | 560 | Glyphosate | 1120 | AMATA | 21 | 100 |
| Bridge 1 | 14 | D10 | 560 | Glyphosate | 1120 | AMATA | 21 | 100 |
| Bridge 1 | 15 | D10 | 1120 | Glyphosate | 1120 | AMATA | 21 | 100 |
| Bridge 1 | 15 | D10 | 1120 | Glyphosate | 1120 | AMATA | 21 | 97 |
| Bridge 1 | 15 | D10 | 1120 | Glyphosate | 1120 | AMATA | 21 | 100 |
| Bridge 1 | 16 | D10 | 0 | Glufosinate | 600 | AMATA | 21 | 100 |
| Bridge 1 | 16 | D10 | 0 | Glufosinate | 600 | AMATA | 21 | 100 |
| Bridge 1 | 16 | D10 | 0 | Glufosinate | 600 | AMATA | 21 | 100 |
| Bridge 1 | 17 | D10 | 560 | Glufosinate | 600 | AMATA | 21 | 100 |
| Bridge 1 | 17 | D10 | 560 | Glufosinate | 600 | AMATA | 21 | 100 |
| Bridge 1 | 17 | D10 | 560 | Glufosinate | 600 | AMATA | 21 | 100 |
| Bridge 1 | 18 | D10 | 1120 | Glufosinate | 600 | AMATA | 21 | 100 |
| Bridge 1 | 18 | D10 | 1120 | Glufosinate | 600 | AMATA | 21 | 99 |
| Bridge 1 | 18 | D10 | 1120 | Glufosinate | 600 | AMATA | 21 | 100 |
| Bridge 1 | 19 | D8 | 560 | none | 0 | AMATA | 21 | 100 |
| Bridge 1 | 19 | D8 | 560 | none | 0 | AMATA | 21 | 100 |
| Bridge 1 | 19 | D8 | 560 | none | 0 | AMATA | 21 | 100 |
| Bridge 1 | 20 | D8 | 1120 | none | 0 | AMATA | 21 | 100 |
| Bridge 1 | 20 | D8 | 1120 | none | | AMATA | 21 | 100 |
| Bridge 1 | 20 | D8 | 1120 | none | 0 | AMATA | 21 | 100 |
| Bridge 1 | 21 | D8 | 560 | diclosulam | 35 | AMATA | 21 | 100 |
| Bridge 1 | 21 | D8 | 560 | diclosulam | 35 | AMATA | 21 | 65 |
| Bridge 1 | 21 | D8 | 560 | diclosulam | 35 | AMATA | 21 | 98 |
| Bridge 1 | 22 | D8 | 1120 | diclosulam | 35 | AMATA | 21 | 100 |
| Bridge 1 | 22 | D8 | 1120 | diclosulam | 35 | AMATA | 21 | 100 |
| Bridge 1 | 22 | D8 | 1120 | diclosulam | 35 | AMATA | 21 | 100 |
| Bridge 1 | 23 | D8 | 560 | Arvlex | 5 | AMATA | 21 | 100 |
| Bridge 1 | 23 | D8 | 560 | Arylex | 5 | AMATA | 21 | 100 |
| Bridge 1 | 23 | D8 | 560 | Arylex | 5 | AMATA | 21 | 100 |
| Bridge 1 | 24 | D8 | 1120 | Arylex | 5 | AMATA | 21 | 100 |
| Bridge 1 | 24 | D8 | 1120 | Arvlex | 5 | AMATA | 21 | 100 |
| Bridge 1 | 24 | D8 | 1120 | Arylex | 5 | AMATA | 21 | 100 |
| Bridge 1 | 25 | D8 | 560 | Mesotrione | 150 | AMATA | 21 | 100 |
| Bridge 1 | 25 | D8 | 560 | Mesotrione | 150 | AMATA | 21 | 100 |
| Bridge 1 | 25 | D8 | 560 | Mesotrione | 150 | AMATA | 21 | 100 |
| Bridge 1 | 26 | D8 | 1120 | Mesotrione | 150 | AMATA | 21 | 100 |
| Bridge 1 | 26 | D8 | 1120 | Mesotrione | 150 | AMATA | 21 | 100 |
| Bridge 1 | 26 | D8 | 1120 | Mesotrione | 150 | AMATA | 21 | 100 |
| Bridge 1 | 27 | D8 | 560 | Glyphosate | 1120 | AMATA | 21 | 100 |
| Bridge 1 | 27 | D8 | 560 | Glyphosate | 1120 | AMATA | 21 | 100 |
| Bridge 1 | 27 | D8 | 560 | Glyphosate | 1120 | AMATA | 21 | 100 |
| Bridge 1 | 28 | D8 | 1120 | Glyphosate | 1120 | AMATA | 21 | 100 |
| Bridge 1 | 28 | D8 | 1120 | Glyphosate | 1120 | AMATA | 21 | 100 |
| Bridge 1 | 28 | D8 | 1120 | Glyphosate | 1120 | AMATA | 21 | 100 |
| Bridge 1 | 29 | D8 | 560 | Glufosinate | 600 | AMATA | 21 | 100 |
| Bridge 1 | 29 | D8 | 560 | Glufosinate | 600 | AMATA | 21 | 100 |
| Bridge 1 | 29 | D8 | 560 | Glufosinate | 600 | AMATA | 21 | 100 |
| Bridge 1 | 30 | D8 | 1120 | Glufosinate | 600 | AMATA | 21 | 100 |
| Bridge 1 | 30 | D8 | 1120 | Glufosinate | 600 | AMATA | 21 | 100 |
| Bridge 1 | 30 | D8 | 1120 | Glufosinate | 600 | AMATA | 21 | 100 |
| Bridge 1 | 31 | D27 | 560 | none | 0 | AMATA | 21 | 100 |
| Bridge 1 | 31 | D27 | 560 | none | 0 | AMATA | 21 | 100 |
| Bridge 1 | 31 | D27 | 560 | none | 0 | AMATA | 21 | 100 |
| Bridge 1 | 32 | D27 | 1120 | none | 0 | AMATA | 21 | 100 |
| Bridge 1 | 32 | D27 | 1120 | none | 0 | AMATA | 21 | 100 |
| Bridge 1 | 32 | D27 | 1120 | none | 0 | AMATA | 21 | 100 |
| Bridge 1 | 33 | D27 | 560 | diclosulam | 35 | AMATA | 21 | 100 |
| Bridge 1 | 33 | D27 | 560 | diclosulam | 35 | AMATA | 21 | 100 |
| Bridge 1 | 33 | D27 | 560 | diclosulam | 35 | AMATA | 21 | 99 |
| Bridge 1 | 34 | D27 | 1120 | diclosulam | 35 | AMATA | 21 | 100 |
| Bridge 1 | 34 | D27 | 1120 | diclosulam | 35 | AMATA | 21 | 100 |
| Bridge 1 | 34 | D27 | 1120 | diclosulam | 35 | AMATA | 21 | 100 |
| Bridge 1 | 35 | D27 | 560 | Arylex | 5 | AMATA | 21 | 100 |
| Bridge 1 | 35 | D27 | 560 | Arylex | 5 | AMATA | 21 | 100 |
| Bridge 1 | 35 | D27 | 560 | Arvlex | 5 | AMATA | 21 | 100 |
| Bridge 1 | 36 | D27 | 1120 | Arylex | 5 | AMATA | 21 | 95 |
| Bridge 1 | 36 | D27 | 1120 | Arylex | 5 | AMATA | 21 | 100 |
| Bridge 1 | 36 | D27 | 1120 | Arylex | 5 | AMATA | 21 | 100 |
| Bridge 1 | 37 | D27 | 560 | Mesotrione | 150 | AMATA | 21 | 100 |
| Bridge 1 | 37 | D27 | 560 | Mesotrione | 150 | AMATA | 21 | 100 |
| Bridge 1 | 37 | D27 | 560 | Mesotrione | 150 | AMATA | 21 | 100 |
| Bridge 1 | 38 | D27 | 1120 | Mesotrione | 150 | AMATA | 21 | 100 |
| Bridge 1 | 38 | D27 | 1120 | Mesotrione | 150 | AMATA | 21 | 100 |
| Bridge 1 | 38 | D27 | 1120 | Mesotrione | 150 | AMATA | 21 | 100 |
| Bridge 1 | 39 | D27 | 560 | Glyphosate | 1120 | AMATA | 21 | 100 |
| Bridge 1 | 39 | D27 | 560 | Glyphosate | 1120 | AMATA | 21 | 100 |
| Bridge 1 | 39 | D27 | 560 | Glyphosate | 1120 | AMATA | 21 | 100 |
| Bridge 1 | 40 | D27 | 1120 | Glyphosate | 1120 | AMATA | 21 | 100 |
| Bridge 1 | 40 | D27 | 1120 | Glyphosate | 1120 | AMATA | 21 | 80 |
| Bridge 1 | 40 | D27 | 1120 | Glyphosate | 1120 | AMATA | 21 | 100 |
| Bridge 1 | 41 | D27 | 560 | Glufosinate | 600 | AMATA | 21 | 100 |
| Bridge 1 | 41 | D27 | 560 | Glufosinate | 600 | AMATA | 21 | 100 |
| Bridge 1 | 41 | D27 | 560 | Glufosinate | 600 | AMATA | 21 | 100 |
| Bridge 1 | 42 | D27 | 1120 | Glufosinate | 600 | AMATA | 21 | 100 |
| Bridge 1 | 42 | D27 | 1120 | Glufosinate | 600 | AMATA | 21 | 100 |
| Bridge 1 | 42 | D27 | 1120 | Glufosinate | 600 | AMATA | 21 | 100 |
| Bridge 1 | 43 | B2 | 1120 | none | | AMATA | 21 | 100 |
| Bridge 1 | 43 | B2 | 1120 | none | | AMATA | 21 | 100 |
| Bridge 1 | 43 | B2 | 1120 | none | | AMATA | 21 | 100 |
| Bridge 1 | 44 | B2 | 1120 | diclosulam | 35 | AMATA | 21 | 100 |
| Bridge 1 | 44 | B2 | 1120 | diclosulam | 35 | AMATA | 21 | 100 |
| Bridge 1 | 44 | B2 | 1120 | diclosulam | 35 | AMATA | 21 | 100 |
| Bridge 1 | 45 | B2 | 1120 | Arylex | 5 | AMATA | 21 | 100 |
| Bridge 1 | 45 | B2 | 1120 | Arylex | 5 | AMATA | 21 | 100 |
| Bridge 1 | 45 | B2 | 1120 | Arylex | 5 | AMATA | 21 | 100 |
| Bridge 1 | 46 | B2 | 1120 | Mesotrione | 150 | AMATA | 21 | 100 |
| Bridge 1 | 46 | B2 | 1120 | Mesotrione | 150 | AMATA | 21 | 100 |
| Bridge 1 | 46 | B2 | 1120 | Mesotrione | 150 | AMATA | 21 | 100 |
| Bridge 1 | 47 | B2 | 1120 | Glyphosate | 1120 | AMATA | 21 | 100 |
| Bridge 1 | 47 | B2 | 1120 | Glyphosate | 1120 | AMATA | 21 | 100 |
| Bridge 1 | 47 | B2 | 1120 | Glyphosate | 1120 | AMATA | 21 | 100 |
| Bridge 1 | 48 | B2 | 1120 | Glufosinate | 600 | AMATA | 21 | 100 |
| Bridge 1 | 48 | B2 | 1120 | Glufosinate | 600 | AMATA | 21 | 100 |
| Bridge 1 | 48 | B2 | 1120 | Glufosinate | 600 | AMATA | 21 | 100 |
| Bridge 1 | 1 | Solv. Ck | 0 | 0 | 0 | CHEAL | 21 | 0 |
| | | untreated | | | | | | |
| Bridge 1 | 1 | Solv. Ck | 0 | 0 | 0 | CHEAL | 21 | 0 |
| | | untreated | | | | | | |
| Bridge 1 | 1 | Solv. Ck | 0 | 0 | 0 | CHEAL | 21 | 0 |
| | | untreated | | | | | | |
| Bridge 1 | 2 | D10 | 560 | none | 0 | CHEAL | 21 | 95 |
| Bridge 1 | 2 | D10 | 560 | none | 0 | CHEAL | 21 | 100 |
| Bridge 1 | 2 | D10 | 560 | none | 0 | CHEAL | 21 | 97 |
| Bridge 1 | 3 | D10 | 1120 | none | 0 | CHEAL | 21 | 95 |
| Bridge 1 | 3 | D10 | 1120 | none | 0 | CHEAL | 21 | 95 |
| Bridge 1 | 3 | D10 | 1120 | none | 0 | CHEAL | 21 | 95 |
| Bridge 1 | 4 | D10 | 0 | diclosulam | 35 | CHEAL | 21 | 30 |
| Bridge 1 | 4 | D10 | 0 | diclosulam | 35 | CHEAL | 21 | 10 |
| Bridge 1 | 4 | D10 | 0 | diclosulam | 35 | CHEAL | 21 | 35 |
| Bridge 1 | 5 | D10 | 560 | diclosulam | 35 | CHEAL | 21 | 95 |
| Bridge 1 | 5 | D10 | 560 | diclosulam | 35 | CHEAL | 21 | 100 |
| Bridge 1 | 5 | D10 | 560 | diclosulam | 35 | CHEAL | 21 | 98 |
| Bridge 1 | 6 | D10 | 1120 | diclosulam | 35 | CHEAL | 21 | 99 |
| Bridge 1 | 6 | D10 | 1120 | diclosulam | 35 | CHEAL | 21 | 100 |
| Bridge 1 | 6 | D10 | 1120 | diclosulam | 35 | CHEAL | 21 | 95 |
| Bridge 1 | 7 | D10 | 0 | Arvlex | 5 | CHEAL | 21 | 100 |
| Bridge 1 | 7 | D10 | 0 | Arylex | 5 | CHEAL | 21 | 100 |
| Bridge 1 | 7 | D10 | 0 | Arylex | 5 | CHEAL | 21 | 100 |
| Bridge 1 | 8 | D10 | 560 | Arylex | 5 | CHEAL | 21 | 95 |
| Bridge 1 | 8 | D10 | 560 | Arylex | 5 | CHEAL | 21 | 97 |
| Bridge 1 | 8 | D10 | 560 | Arylex | 5 | CHEAL | 21 | 100 |
| Bridge 1 | 9 | D10 | 1120 | Arvlex | 5 | CHEAL | 21 | 95 |
| Bridge 1 | 9 | D10 | 1120 | Arylex | 5 | CHEAL | 21 | 95 |
| Bridge 1 | 9 | D10 | 1120 | Arylex | 5 | CHEAL | 21 | 95 |
| Bridge 1 | 10 | D10 | 0 | Mesotrione | 150 | CHEAL | 21 | 100 |
| Bridge 1 | 10 | D10 | 0 | Mesotrione | 150 | CHEAL | 21 | 100 |
| Bridge 1 | 10 | D10 | 0 | Mesotrione | 150 | CHEAL | 21 | 100 |
| Bridge 1 | 11 | D10 | 560 | Mesotrione | 150 | CHEAL | 21 | 100 |
| Bridge 1 | 11 | D10 | 560 | Mesotrione | 150 | CHEAL | 21 | 100 |
| Bridge 1 | 11 | D10 | 560 | Mesotrione | 150 | CHEAL | 21 | 100 |
| Bridge 1 | 12 | D10 | 1120 | Mesotrione | 150 | CHEAL | 21 | 100 |
| Bridge 1 | 12 | D10 | 1120 | Mesotrione | 150 | CHEAL | 21 | 100 |
| Bridge 1 | 12 | D10 | 1120 | Mesotrione | 150 | CHEAL | 21 | 100 |
| Bridge 1 | 13 | D10 | 0 | Glyphosate | 1120 | CHEAL | 21 | 99 |
| Bridge 1 | 13 | D10 | 0 | Glyphosate | 1120 | CHEAL | 21 | 95 |
| Bridge 1 | 13 | D10 | 0 | Glyphosate | 1120 | CHEAL | 21 | 98 |
| Bridge 1 | 14 | D10 | 560 | Glyphosate | 1120 | CHEAL | 21 | 98 |
| Bridge 1 | 14 | D10 | 560 | Glyphosate | 1120 | CHEAL | 21 | 95 |
| Bridge 1 | 14 | D10 | 560 | Glyphosate | 1120 | CHEAL | 21 | 95 |
| Bridge 1 | 15 | D10 | 1120 | Glyphosate | 1120 | CHEAL | 21 | 98 |
| Bridge 1 | 15 | D10 | 1120 | Glyphosate | 1120 | CHEAL | 21 | 97 |
| Bridge 1 | 15 | D10 | 1120 | Glyphosate | 1120 | CHEAL | 21 | 97 |
| Bridge 1 | 16 | D10 | 0 | Glufosinate | 600 | CHEAL | 21 | 50 |
| Bridge 1 | 16 | D10 | 0 | Glufosinate | 600 | CHEAL | 21 | 100 |
| Bridge 1 | 16 | D10 | 0 | Glufosinate | 600 | CHEAL | 21 | 100 |
| Bridge 1 | 17 | D10 | 560 | Glufosinate | 600 | CHEAL | 21 | 100 |
| Bridge 1 | 17 | D10 | 560 | Glufosinate | 600 | CHEAL | 21 | 100 |
| Bridge 1 | 17 | D10 | 560 | Glufosinate | 600 | CHEAL | 21 | 100 |
| Bridge 1 | 18 | D10 | 1120 | Glufosinate | 600 | CHEAL | 21 | 100 |
| Bridge 1 | 18 | D10 | 1120 | Glufosinate | 600 | CHEAL | 21 | 100 |
| Bridge 1 | 18 | D10 | 1120 | Glufosinate | 600 | CHEAL | 21 | 85 |
| Bridge 1 | 19 | D8 | 560 | none | 0 | CHEAL | 21 | 95 |
| Bridge 1 | 19 | D8 | 560 | none | 0 | CHEAL | 21 | 95 |
| Bridge 1 | 19 | D8 | 560 | none | 0 | CHEAL | 21 | 95 |
| Bridge 1 | 20 | D8 | 1120 | none | 0 | CHEAL | 21 | 100 |
| Bridge 1 | 20 | D8 | 1120 | none | 0 | CHEAL | 21 | 100 |
| Bridge 1 | 20 | D8 | 1120 | none | 0 | CHEAL | 21 | 100 |
| Bridge 1 | 21 | D8 | 560 | diclosulam | 35 | CHEAL | 21 | 90 |
| Bridge 1 | 21 | D8 | 560 | diclosulam | 35 | CHEAL | 21 | 90 |
| Bridge 1 | 21 | D8 | 560 | diclosulam | 35 | CHEAL | 21 | 95 |
| Bridge 1 | 22 | D8 | 1120 | diclosulam | 35 | CHEAL | 21 | 95 |
| Bridge 1 | 22 | D8 | 1120 | diclosulam | 35 | CHEAL | 21 | 100 |
| Bridge 1 | 22 | D8 | 1120 | diclosulam | 35 | CHEAL | 21 | 90 |
| Bridge 1 | 23 | D8 | 560 | Arylex | 5 | CHEAL | 21 | 100 |
| Bridge 1 | 23 | D8 | 560 | Arylex | 5 | CHEAL | 21 | 100 |
| Bridge 1 | 23 | D8 | 560 | Arvlex | 5 | CHEAL | 21 | 100 |
| Bridge 1 | 24 | D8 | 1120 | Arylex | 5 | CHEAL | 21 | 100 |
| Bridge 1 | 24 | D8 | 1120 | Arylex | 5 | CHEAL | 21 | 88 |
| Bridge 1 | 24 | D8 | 1120 | Arylex | 5 | CHEAL | 21 | 100 |
| Bridge 1 | 25 | D8 | 560 | Mesotrione | 150 | CHEAL | 21 | 100 |
| Bridge 1 | 25 | D8 | 560 | Mesotrione | 150 | CHEAL | 21 | 100 |
| Bridge 1 | 25 | D8 | 560 | Mesotrione | 150 | CHEAL | 21 | 100 |
| Bridge 1 | 26 | D8 | 1120 | Mesotrione | 150 | CHEAL | 21 | 100 |
| Bridge 1 | 26 | D8 | 1120 | Mesotrione | 150 | CHEAL | 21 | 100 |
| Bridge 1 | 26 | D8 | 1120 | Mesotrione | 150 | CHEAL | 21 | 100 |
| Bridge 1 | 27 | D8 | 560 | Glyphosate | 1120 | CHEAL | 21 | 100 |
| Bridge 1 | 27 | D8 | 560 | Glyphosate | 1120 | CHEAL | 21 | 98 |
| Bridge 1 | 27 | D8 | 560 | Glyphosate | 1120 | CHEAL | 21 | 95 |
| Bridge 1 | 28 | D8 | 1120 | Glyphosate | 1120 | CHEAL | 21 | 95 |
| Bridge 1 | 28 | D8 | 1120 | Glyphosate | 1120 | CHEAL | 21 | 100 |
| Bridge 1 | 28 | D8 | 1120 | Glyphosate | 1120 | CHEAL | 21 | 97 |
| Bridge 1 | 29 | D8 | 560 | Glufosinate | 600 | CHEAL | 21 | 100 |
| Bridge 1 | 29 | D8 | 560 | Glufosinate | 600 | CHEAL | 21 | 100 |
| Bridge 1 | 29 | D8 | 560 | Glufosinate | 600 | CHEAL | 21 | 99 |
| Bridge 1 | 30 | D8 | 1120 | Glufosinate | 600 | CHEAL | 21 | 100 |
| Bridge 1 | 30 | D8 | 1120 | Glufosinate | 600 | CHEAL | 21 | 100 |
| Bridge 1 | 30 | D8 | 1120 | Glufosinate | 600 | CHEAL | 21 | 100 |
| Bridge 1 | 31 | D27 | 560 | none | 0 | CHEAL | 21 | 98 |
| Bridge 1 | 31 | D27 | 560 | none | 0 | CHEAL | 21 | 98 |
| Bridge 1 | 31 | D27 | 560 | none | 0 | CHEAL | 21 | 90 |
| Bridge 1 | 32 | D27 | 1120 | none | 0 | CHEAL | 21 | 100 |
| Bridge 1 | 32 | D27 | 1120 | none | 0 | CHEAL | 21 | 98 |
| Bridge 1 | 32 | D27 | 1120 | none | 0 | CHEAL | 21 | 98 |
| Bridge 1 | 33 | D27 | 560 | diclosulam | 35 | CHEAL | 21 | 90 |
| Bridge 1 | 33 | D27 | 560 | diclosulam | 35 | CHEAL | 21 | 90 |
| Bridge 1 | 33 | D27 | 560 | diclosulam | 35 | CHEAL | 21 | 95 |
| Bridge 1 | 34 | D27 | 1120 | diclosulam | 35 | CHEAL | 21 | 95 |
| Bridge 1 | 34 | D27 | 1120 | diclosulam | 35 | CHEAL | 21 | 100 |
| Bridge 1 | 34 | D27 | 1120 | diclosulam | 35 | CHEAL | 21 | 95 |
| Bridge 1 | 35 | D27 | 560 | Arylex | 5 | CHEAL | 21 | 100 |
| Bridge 1 | 35 | D27 | 560 | Arylex | 5 | CHEAL | 21 | 100 |
| Bridge 1 | 35 | D27 | 560 | Arylex | 5 | CHEAL | 21 | 100 |
| Bridge 1 | 36 | D27 | 1120 | Arylex | 5 | CHEAL | 21 | 100 |
| Bridge 1 | 36 | D27 | 1120 | Arvlex | 5 | CHEAL | 21 | 100 |
| Bridge 1 | 36 | D27 | 1120 | Arylex | 5 | CHEAL | 21 | 100 |
| Bridge 1 | 37 | D27 | 560 | Mesotrione | 150 | CHEAL | 21 | 100 |
| Bridge 1 | 37 | D27 | 560 | Mesotrione | 150 | CHEAL | 21 | 100 |
| Bridge 1 | 37 | D27 | 560 | Mesotrione | 150 | CHEAL | 21 | 100 |
| Bridge 1 | 38 | D27 | 1120 | Mesotrione | 150 | CHEAL | 21 | 100 |
| Bridge 1 | 38 | D27 | 1120 | Mesotrione | 150 | CHEAL | 21 | 100 |
| Bridge 1 | 38 | D27 | 1120 | Mesotrione | 150 | CHEAL | 21 | 100 |
| Bridge 1 | 39 | D27 | 560 | Glyphosate | 1120 | CHEAL | 21 | 95 |
| Bridge 1 | 39 | D27 | 560 | Glyphosate | 1120 | CHEAL | 21 | 100 |
| Bridge 1 | 39 | D27 | 560 | Glyphosate | 1120 | CHEAL | 21 | 97 |
| Bridge 1 | 40 | D27 | 1120 | Glyphosate | 1120 | CHEAL | 21 | 95 |
| Bridge 1 | 40 | D27 | 1120 | Glyphosate | 1120 | CHEAL | 21 | 99 |
| Bridge 1 | 40 | D27 | 1120 | Glyphosate | 1120 | CHEAL | 21 | 100 |
| Bridge 1 | 41 | D27 | 560 | Glufosinate | 600 | CHEAL | 21 | 100 |
| Bridge 1 | 41 | D27 | 560 | Glufosinate | 600 | CHEAL | 21 | 100 |
| Bridge 1 | 41 | D27 | 560 | Glufosinate | 600 | CHEAL | 21 | 98 |
| Bridge 1 | 42 | D27 | 1120 | Glufosinate | 600 | CHEAL | 21 | 100 |
| Bridge 1 | 42 | D27 | 1120 | Glufosinate | 600 | CHEAL | 21 | 100 |
| Bridge 1 | 42 | D27 | 1120 | Glufosinate | 600 | CHEAL | 21 | 100 |
| Bridge 1 | 43 | B2 | 1120 | none | | CHEAL | 21 | 90 |
| Bridge 1 | 43 | B2 | 1120 | none | | CHEAL | 21 | 100 |
| Bridge 1 | 43 | B2 | 1120 | none | | CHEAL | 21 | 100 |
| Bridge 1 | 44 | B2 | 1120 | diclosulam | 35 | CHEAL | 21 | 95 |
| Bridge 1 | 44 | B2 | 1120 | diclosulam | 35 | CHEAL | 21 | 99 |
| Bridge 1 | 44 | B2 | 1120 | diclosulam | 35 | CHEAL | 21 | 100 |
| Bridge 1 | 45 | B2 | 1120 | Arvlex | 5 | CHEAL | 21 | 100 |
| Bridge 1 | 45 | B2 | 1120 | Arylex | 5 | CHEAL | 21 | 100 |
| Bridge 1 | 45 | B2 | 1120 | Arvlex | 5 | CHEAL | 21 | 100 |
| Bridge 1 | 46 | B2 | 1120 | Mesotrione | 150 | CHEAL | 21 | 100 |
| Bridge 1 | 46 | B2 | 1120 | Mesotrione | 150 | CHEAL | 21 | 100 |
| Bridge 1 | 46 | B2 | 1120 | Mesotrione | 150 | CHEAL | 21 | 100 |
| Bridge 1 | 47 | B2 | 1120 | Glyphosate | 1120 | CHEAL | 21 | 100 |
| Bridge 1 | 47 | B2 | 1120 | Glyphosate | 1120 | CHEAL | 21 | 100 |
| Bridge 1 | 47 | B2 | 1120 | Glyphosate | 1120 | CHEAL | 21 | 95 |
| Bridge 1 | 48 | B2 | 1120 | Glufosinate | 600 | CHEAL | 21 | 100 |
| Bridge 1 | 48 | B2 | 1120 | Glufosinate | 600 | CHEAL | 21 | 100 |
| Bridge 1 | 48 | B2 | 1120 | Glufosinate | 600 | CHEAL | 21 | 100 |
| Bridge 1 | 1 | Solv. Ck | 0 | 0 | 0 | ERICA | 21 | 0 |
| | | untreated | | | | | | |
| Bridge 1 | 1 | Solv. Ck | 0 | 0 | 0 | ERICA | 21 | 0 |
| | | untreated | | | | | | |
| Bridge 1 | 1 | Solv. Ck | 0 | 0 | 0 | ERICA | 21 | 0 |
| | | untreated | | | | | | |
| Bridge 1 | 2 | D10 | 560 | none | 0 | ERICA | 21 | 95 |
| Bridge 1 | 2 | D10 | 560 | none | 0 | ERICA | 21 | 85 |
| Bridge 1 | 2 | D10 | 560 | none | 0 | ERICA | 21 | 90 |
| Bridge 1 | 3 | D10 | 1120 | none | 0 | ERICA | 21 | 98 |
| Bridge 1 | 3 | D10 | 1120 | none | 0 | ERICA | 21 | 90 |
| Bridge 1 | 3 | D10 | 1120 | none | 0 | ERICA | 21 | 90 |
| Bridge 1 | 4 | D10 | 0 | diclosulam | 35 | ERICA | 21 | 99 |
| Bridge 1 | 4 | D10 | 0 | diclosulam | 35 | ERICA | 21 | 100 |
| Bridge 1 | 4 | D10 | 0 | diclosulam | 35 | ERICA | 21 | 100 |
| Bridge 1 | 5 | D10 | 560 | diclosulam | 35 | ERICA | 21 | 100 |
| Bridge 1 | 5 | D10 | 560 | diclosulam | 35 | ERICA | 21 | 100 |
| Bridge 1 | 5 | D10 | 560 | diclosulam | 35 | ERICA | 21 | 95 |
| Bridge 1 | 6 | D10 | 1120 | diclosulam | 35 | ERICA | 21 | 100 |
| Bridge 1 | 6 | D10 | 1120 | diclosulam | 35 | ERICA | 21 | 100 |
| Bridge 1 | 6 | D10 | 1120 | diclosulam | 35 | ERICA | 21 | 100 |
| Bridge 1 | 7 | D10 | 0 | Arylex | 5 | ERICA | 21 | 90 |
| Bridge 1 | 7 | D10 | 0 | Arylex | 5 | ERICA | 21 | 85 |
| Bridge 1 | 7 | D10 | 0 | Arylex | 5 | ERICA | 21 | 85 |
| Bridge 1 | 8 | D10 | 560 | Arylex | 5 | ERICA | 21 | 90 |
| Bridge 1 | 8 | D10 | 560 | Arylex | 5 | ERICA | 21 | 90 |
| Bridge 1 | 8 | D10 | 560 | Arylex | 5 | ERICA | 21 | 90 |
| Bridge 1 | 9 | D10 | 1120 | Arylex | 5 | ERICA | 21 | 95 |
| Bridge 1 | 9 | D10 | 1120 | Arylex | 5 | ERICA | 21 | 95 |
| Bridge 1 | 9 | D10 | 1120 | Arvlex | 5 | ERICA | 21 | 90 |
| Bridge 1 | 10 | D10 | 0 | Mesotrione | 150 | ERICA | 21 | 75 |
| Bridge 1 | 10 | D10 | 0 | Mesotrione | 150 | ERICA | 21 | 75 |
| Bridge 1 | 10 | D10 | 0 | Mesotrione | 150 | ERICA | 21 | 70 |
| Bridge 1 | 11 | D10 | 560 | Mesotrione | 150 | ERICA | 21 | 100 |
| Bridge 1 | 11 | D10 | 560 | Mesotrione | 150 | ERICA | 21 | 98 |
| Bridge 1 | 11 | D10 | 560 | Mesotrione | 150 | ERICA | 21 | 100 |
| Bridge 1 | 12 | D10 | 1120 | Mesotrione | 150 | ERICA | 21 | 100 |
| Bridge 1 | 12 | D10 | 1120 | Mesotrione | 150 | ERICA | 21 | 100 |
| Bridge 1 | 12 | D10 | 1120 | Mesotrione | 150 | ERICA | 21 | 100 |
| Bridge 1 | 13 | D10 | 0 | Glyphosate | 1120 | ERICA | 21 | 100 |
| Bridge 1 | 13 | D10 | 0 | Glyphosate | 1120 | ERICA | 21 | 100 |
| Bridge 1 | 13 | D10 | 0 | Glyphosate | 1120 | ERICA | 21 | |
| Bridge 1 | 14 | D10 | 560 | Glyphosate | 1120 | ERICA | 21 | 99 |
| Bridge 1 | 14 | D10 | 560 | Glyphosate | 1120 | ERICA | 21 | 100 |
| Bridge 1 | 14 | D10 | 560 | Glyphosate | 1120 | ERICA | 21 | |
| Bridge 1 | 15 | D10 | 1120 | Glyphosate | 1120 | ERICA | 21 | 75 |
| Bridge 1 | 15 | D10 | 1120 | Glyphosate | 1120 | ERICA | 21 | 100 |
| Bridge 1 | 15 | D10 | 1120 | Glyphosate | 1120 | ERICA | 21 | |
| Bridge 1 | 16 | D10 | 0 | Glufosinate | 600 | ERICA | 21 | 85 |
| Bridge 1 | 16 | D10 | 0 | Glufosinate | 600 | ERICA | 21 | 100 |
| Bridge 1 | 16 | D10 | 0 | Glufosinate | 600 | ERICA | 21 | 100 |
| Bridge 1 | 17 | D10 | 560 | Glufosinate | 600 | ERICA | 21 | 100 |
| Bridge 1 | 17 | D10 | 560 | Glufosinate | 600 | ERICA | 21 | 100 |
| Bridge 1 | 17 | D10 | 560 | Glufosinate | 600 | ERICA | 21 | 100 |
| Bridge 1 | 18 | D10 | 1120 | Glufosinate | 600 | ERICA | 21 | 100 |
| Bridge 1 | 18 | D10 | 1120 | Glufosinate | 600 | ERICA | 21 | 100 |
| Bridge 1 | 18 | D10 | 1120 | Glufosinate | 600 | ERICA | 21 | 100 |
| Bridge 1 | 19 | D8 | 560 | none | 0 | ERICA | 21 | 95 |
| Bridge 1 | 19 | D8 | 560 | none | 0 | ERICA | 21 | 90 |
| Bridge 1 | 19 | D8 | 560 | none | 0 | ERICA | 21 | 90 |
| Bridge 1 | 20 | D8 | 1120 | none | 0 | ERICA | 21 | 100 |
| Bridge 1 | 20 | D8 | 1120 | none | 0 | ERICA | 21 | 90 |
| Bridge 1 | 20 | D8 | 1120 | none | 0 | ERICA | 21 | 90 |
| Bridge 1 | 21 | D8 | 560 | diclosulam | 35 | ERICA | 21 | 100 |
| Bridge 1 | 21 | D8 | 560 | diclosulam | 35 | ERICA | 21 | 100 |
| Bridge 1 | 21 | D8 | 560 | diclosulam | 35 | ERICA | 21 | 100 |
| Bridge 1 | 22 | D8 | 1120 | diclosulam | 35 | ERICA | 21 | 100 |
| Bridge 1 | 22 | D8 | 1120 | diclosulam | 35 | ERICA | 21 | 100 |
| Bridge 1 | 22 | D8 | 1120 | diclosulam | 35 | ERICA | 21 | 100 |
| Bridge 1 | 23 | D8 | 560 | Arylex | 5 | ERICA | 21 | 90 |
| Bridge 1 | 23 | D8 | 560 | Arylex | 5 | ERICA | 21 | 95 |
| Bridge 1 | 23 | D8 | 560 | Arylex | 5 | ERICA | 21 | 90 |
| Bridge 1 | 24 | D8 | 1120 | Arylex | 5 | ERICA | 21 | 85 |
| Bridge 1 | 24 | D8 | 1120 | Arylex | 5 | ERICA | 21 | 90 |
| Bridge 1 | 24 | D8 | 1120 | Arylex | 5 | ERICA | 21 | 90 |
| Bridge 1 | 25 | D8 | 560 | Mesotrione | 150 | ERICA | 21 | 100 |
| Bridge 1 | 25 | D8 | 560 | Mesotrione | 150 | ERICA | 21 | 99 |
| Bridge 1 | 25 | D8 | 560 | Mesotrione | 150 | ERICA | 21 | 98 |
| Bridge 1 | 26 | D8 | 1120 | Mesotrione | 150 | ERICA | 21 | 99 |
| Bridge 1 | 26 | D8 | 1120 | Mesotrione | 150 | ERICA | 21 | 100 |
| Bridge 1 | 26 | D8 | 1120 | Mesotrione | 150 | ERICA | 21 | 100 |
| Bridge 1 | 27 | D8 | 560 | Glyphosate | 1120 | ERICA | 21 | 80 |
| Bridge 1 | 27 | D8 | 560 | Glyphosate | 1120 | ERICA | 21 | 100 |
| Bridge 1 | 27 | D8 | 560 | Glyphosate | 1120 | ERICA | 21 | |
| Bridge 1 | 28 | D8 | 1120 | Glyphosate | 1120 | ERICA | 21 | 95 |
| Bridge 1 | 28 | D8 | 1120 | Glyphosate | 1120 | ERICA | 21 | 90 |
| Bridge 1 | 28 | D8 | 1120 | Glyphosate | 1120 | ERICA | 21 | |
| Bridge 1 | 29 | D8 | 560 | Glufosinate | 600 | ERICA | 21 | 100 |
| Bridge 1 | 29 | D8 | 560 | Glufosinate | 600 | ERICA | 21 | 90 |
| Bridge 1 | 29 | D8 | 560 | Glufosinate | 600 | ERICA | 21 | 85 |
| Bridge 1 | 30 | D8 | 1120 | Glufosinate | 600 | ERICA | 21 | 100 |
| Bridge 1 | 30 | D8 | 1120 | Glufosinate | 600 | ERICA | 21 | 100 |
| Bridge 1 | 30 | D8 | 1120 | Glufosinate | 600 | ERICA | 21 | 98 |
| Bridge 1 | 31 | D27 | 560 | none | 0 | ERICA | 21 | 85 |
| Bridge 1 | 31 | D27 | 560 | none | 0 | ERICA | 21 | 85 |
| Bridge 1 | 31 | D27 | 560 | none | 0 | ERICA | 21 | 85 |
| Bridge 1 | 32 | D27 | 1120 | none | 0 | ERICA | 21 | 90 |
| Bridge 1 | 32 | D27 | 1120 | none | 0 | ERICA | 21 | 85 |
| Bridge 1 | 32 | D27 | 1120 | none | 0 | ERICA | 21 | 97 |
| Bridge 1 | 33 | D27 | 560 | diclosulam | 35 | ERICA | 21 | 100 |
| Bridge 1 | 33 | D27 | 560 | diclosulam | 35 | ERICA | 21 | 100 |
| Bridge 1 | 33 | D27 | 560 | diclosulam | 35 | ERICA | 21 | 99 |
| Bridge 1 | 34 | D27 | 1120 | diclosulam | 35 | ERICA | 21 | 100 |
| Bridge 1 | 34 | D27 | 1120 | diclosulam | 35 | ERICA | 21 | 100 |
| Bridge 1 | 34 | D27 | 1120 | diclosulam | 35 | ERICA | 21 | 98 |
| Bridge 1 | 35 | D27 | 560 | Arylex | 5 | ERICA | 21 | 85 |
| Bridge 1 | 35 | D27 | 560 | Arylex | 5 | ERICA | 21 | 90 |
| Bridge 1 | 35 | D27 | 560 | Arylex | 5 | ERICA | 21 | 85 |
| Bridge 1 | 36 | D27 | 1120 | Arylex | 5 | ERICA | 21 | 90 |
| Bridge 1 | 36 | D27 | 1120 | Arylex | 5 | ERICA | 21 | 90 |
| Bridge 1 | 36 | D27 | 1120 | Arylex | 5 | ERICA | 21 | 85 |
| Bridge 1 | 37 | D27 | 560 | Mesotrione | 150 | ERICA | 21 | 98 |
| Bridge 1 | 37 | D27 | 560 | Mesotrione | 150 | ERICA | 21 | 100 |
| Bridge 1 | 37 | D27 | 560 | Mesotrione | 150 | ERICA | 21 | 95 |
| Bridge 1 | 38 | D27 | 1120 | Mesotrione | 150 | ERICA | 21 | 100 |
| Bridge 1 | 38 | D27 | 1120 | Mesotrione | 150 | ERICA | 21 | 100 |
| Bridge 1 | 38 | D27 | 1120 | Mesotrione | 150 | ERICA | 21 | 100 |
| Bridge 1 | 39 | D27 | 560 | Glyphosate | 1120 | ERICA | 21 | 100 |
| Bridge 1 | 39 | D27 | 560 | Glyphosate | 1120 | ERICA | 21 | 95 |
| Bridge 1 | 39 | D27 | 560 | Glyphosate | 1120 | ERICA | 21 | |
| Bridge 1 | 40 | D27 | 1120 | Glyphosate | 1120 | ERICA | 21 | 100 |
| Bridge 1 | 40 | D27 | 1120 | Glyphosate | 1120 | ERICA | 21 | 100 |
| Bridge 1 | 40 | D27 | 1120 | Glyphosate | 1120 | ERICA | 21 | |
| Bridge 1 | 41 | D27 | 560 | Glufosinate | 600 | ERICA | 21 | 100 |
| Bridge 1 | 41 | D27 | 560 | Glufosinate | 600 | ERICA | 21 | 100 |
| Bridge 1 | 41 | D27 | 560 | Glufosinate | 600 | ERICA | 21 | 95 |
| Bridge 1 | 42 | D27 | 1120 | Glufosinate | 600 | ERICA | 21 | 100 |
| Bridge 1 | 42 | D27 | 1120 | Glufosinate | 600 | ERICA | 21 | 98 |
| Bridge 1 | 42 | D27 | 1120 | Glufosinate | 600 | ERICA | 21 | 90 |
| Bridge 1 | 43 | B2 | 1120 | none | | ERICA | 21 | 95 |
| Bridge 1 | 43 | B2 | 1120 | none | | ERICA | 21 | 90 |
| Bridge 1 | 43 | B2 | 1120 | none | | ERICA | 21 | 100 |
| Bridge 1 | 44 | B2 | 1120 | diclosulam | 35 | ERICA | 21 | 100 |
| Bridge 1 | 44 | B2 | 1120 | diclosulam | 35 | ERICA | 21 | 100 |
| Bridge 1 | 44 | B2 | 1120 | diclosulam | 35 | ERICA | 21 | 98 |
| Bridge 1 | 45 | B2 | 1120 | Arylex | 5 | ERICA | 21 | 90 |
| Bridge 1 | 45 | B2 | 1120 | Arylex | 5 | ERICA | 21 | 90 |
| Bridge 1 | 45 | B2 | 1120 | Arylex | 5 | ERICA | 21 | 95 |
| Bridge 1 | 46 | B2 | 1120 | Mesotrione | 150 | ERICA | 21 | 100 |
| Bridge 1 | 46 | B2 | 1120 | Mesotrione | 150 | ERICA | 21 | 100 |
| Bridge 1 | 46 | B2 | 1120 | Mesotrione | 150 | ERICA | 21 | 98 |
| Bridge 1 | 47 | B2 | 1120 | Glyphosate | 1120 | ERICA | 21 | 98 |
| Bridge 1 | 47 | B2 | 1120 | Glyphosate | 1120 | ERICA | 21 | 98 |
| Bridge 1 | 47 | B2 | 1120 | Glyphosate | 1120 | ERICA | 21 | |
| Bridge 1 | 48 | B2 | 1120 | Glufosinate | 600 | ERICA | 21 | 90 |
| Bridge 1 | 48 | B2 | 1120 | Glufosinate | 600 | ERICA | 21 | 100 |
| Bridge 1 | 48 | B2 | 1120 | Glufosinate | 600 | ERICA | 21 | 98 |
| Bridge 1 | 1 | Solv. Ck | 0 | 0 | 0 | ZEAMX | 21 | 0 |
| | | untreated | | | | | | |
| Bridge 1 | 1 | Solv. Ck | 0 | 0 | 0 | ZEAMX | 21 | 0 |
| | | untreated | | | | | | |
| Bridge 1 | 1 | Solv. Ck | 0 | 0 | 0 | ZEAMX | 21 | 0 |
| | | untreated | | | | | | |
| Bridge 1 | 2 | D10 | 560 | none | 0 | ZEAMX | 21 | 5 |
| Bridge 1 | 2 | D10 | 560 | none | 0 | ZEAMX | 21 | 0 |
| Bridge 1 | 2 | D10 | 560 | none | 0 | ZEAMX | 21 | 5 |
| Bridge 1 | 3 | D10 | 1120 | none | 0 | ZEAMX | 21 | 10 |
| Bridge 1 | 3 | D10 | 1120 | none | 0 | ZEAMX | 21 | 10 |
| Bridge 1 | 3 | D10 | 1120 | none | 0 | ZEAMX | 21 | 10 |
| Bridge 1 | 4 | D10 | 0 | diclosulam | 35 | ZEAMX | 21 | 70 |
| Bridge 1 | 4 | D10 | 0 | diclosulam | 35 | ZEAMX | 21 | 70 |
| Bridge 1 | 4 | D10 | 0 | diclosulam | 35 | ZEAMX | 21 | 70 |
| Bridge 1 | 5 | D10 | 560 | diclosulam | 35 | ZEAMX | 21 | 50 |
| Bridge 1 | 5 | D10 | 560 | diclosulam | 35 | ZEAMX | 21 | 65 |
| Bridge 1 | 5 | D10 | 560 | diclosulam | 35 | ZEAMX | 21 | 65 |
| Bridge 1 | 6 | D10 | 1120 | diclosulam | 35 | ZEAMX | 21 | 50 |
| Bridge 1 | 6 | D10 | 1120 | diclosulam | 35 | ZEAMX | 21 | 65 |
| Bridge 1 | 6 | D10 | 1120 | diclosulam | 35 | ZEAMX | 21 | 60 |
| Bridge 1 | 7 | D10 | 0 | Arylex | 5 | ZEAMX | 21 | 65 |
| Bridge 1 | 7 | D10 | 0 | Arylex | 5 | ZEAMX | 21 | 65 |
| Bridge 1 | 7 | D10 | 0 | Arvlex | 5 | ZEAMX | 21 | 60 |
| Bridge 1 | 8 | D10 | 560 | Arylex | 5 | ZEAMX | 21 | 5 |
| Bridge 1 | 8 | D10 | 560 | Arvlex | 5 | ZEAMX | 21 | 5 |
| Bridge 1 | 8 | D10 | 560 | Arylex | 5 | ZEAMX | 21 | 10 |
| Bridge 1 | 9 | D10 | 1120 | Arylex | 5 | ZEAMX | 21 | 10 |
| Bridge 1 | 9 | D10 | 1120 | Arylex | 5 | ZEAMX | 21 | 0 |
| Bridge 1 | 9 | D10 | 1120 | Arylex | 5 | ZEAMX | 21 | 20 |
| Bridge 1 | 10 | D10 | 0 | Mesotrione | 150 | ZEAMX | 21 | 0 |
| Bridge 1 | 10 | D10 | 0 | Mesotrione | 150 | ZEAMX | 21 | 5 |
| Bridge 1 | 10 | D10 | 0 | Mesotrione | 150 | ZEAMX | 21 | 0 |
| Bridge 1 | 11 | D10 | 560 | Mesotrione | 150 | ZEAMX | 21 | 5 |
| Bridge 1 | 11 | D10 | 560 | Mesotrione | 150 | ZEAMX | 21 | 5 |
| Bridge 1 | 11 | D10 | 560 | Mesotrione | 150 | ZEAMX | 21 | 10 |
| Bridge 1 | 12 | D10 | 1120 | Mesotrione | 150 | ZEAMX | 21 | 5 |
| Bridge 1 | 12 | D10 | 1120 | Mesotrione | 150 | ZEAMX | 21 | 15 |
| Bridge 1 | 12 | D10 | 1120 | Mesotrione | 150 | ZEAMX | 21 | 10 |
| Bridge 1 | 13 | D10 | 0 | Glyphosate | 1120 | ZEAMX | 21 | 75 |
| Bridge 1 | 13 | D10 | 0 | Glyphosate | 1120 | ZEAMX | 21 | 65 |
| Bridge 1 | 13 | D10 | 0 | Glyphosate | 1120 | ZEAMX | 21 | 70 |
| Bridge 1 | 14 | D10 | 560 | Glyphosate | 1120 | ZEAMX | 21 | 70 |
| Bridge 1 | 14 | D10 | 560 | Glyphosate | 1120 | ZEAMX | 21 | 70 |
| Bridge 1 | 14 | D10 | 560 | Glyphosate | 1120 | ZEAMX | 21 | 75 |
| Bridge 1 | 15 | D10 | 1120 | Glyphosate | 1120 | ZEAMX | 21 | 75 |
| Bridge 1 | 15 | D10 | 1120 | Glyphosate | 1120 | ZEAMX | 21 | 70 |
| Bridge 1 | 15 | D10 | 1120 | Glyphosate | 1120 | ZEAMX | 21 | 75 |
| Bridge 1 | 16 | D10 | 0 | Glufosinate | 600 | ZEAMX | 21 | 15 |
| Bridge 1 | 16 | D10 | 0 | Glufosinate | 600 | ZEAMX | 21 | 15 |
| Bridge 1 | 16 | D10 | 0 | Glufosinate | 600 | ZEAMX | 21 | 5 |
| Bridge 1 | 17 | D10 | 560 | Glufosinate | 600 | ZEAMX | 21 | 60 |
| Bridge 1 | 17 | D10 | 560 | Glufosinate | 600 | ZEAMX | 21 | 55 |
| Bridge 1 | 17 | D10 | 560 | Glufosinate | 600 | ZEAMX | 21 | 15 |
| Bridge 1 | 18 | D10 | 1120 | Glufosinate | 600 | ZEAMX | 21 | 65 |
| Bridge 1 | 18 | D10 | 1120 | Glufosinate | 600 | ZEAMX | 21 | 20 |
| Bridge 1 | 18 | D10 | 1120 | Glufosinate | 600 | ZEAMX | 21 | 10 |
| Bridge 1 | 19 | D8 | 560 | none | 0 | ZEAMX | 21 | 5 |
| Bridge 1 | 19 | D8 | 560 | none | 0 | ZEAMX | 21 | 5 |
| Bridge 1 | 19 | D8 | 560 | none | 0 | ZEAMX | 21 | 10 |
| Bridge 1 | 20 | D8 | 1120 | none | 0 | ZEAMX | 21 | 5 |
| Bridge 1 | 20 | D8 | 1120 | none | 0 | ZEAMX | 21 | 5 |
| Bridge 1 | 20 | D8 | 1120 | none | 0 | ZEAMX | 21 | 10 |
| Bridge 1 | 21 | D8 | 560 | diclosulam | 35 | ZEAMX | 21 | 60 |
| Bridge 1 | 21 | D8 | 560 | diclosulam | 35 | ZEAMX | 21 | 55 |
| Bridge 1 | 21 | D8 | 560 | diclosulam | 35 | ZEAMX | 21 | 50 |
| Bridge 1 | 22 | D8 | 1120 | diclosulam | 35 | ZEAMX | 21 | 55 |
| Bridge 1 | 22 | D8 | 1120 | diclosulam | 35 | ZEAMX | 21 | 55 |
| Bridge 1 | 22 | D8 | 1120 | diclosulam | 35 | ZEAMX | 21 | 60 |
| Bridge 1 | 23 | D8 | 560 | Arylex | 5 | ZEAMX | 21 | 5 |
| Bridge 1 | 23 | D8 | 560 | Arylex | 5 | ZEAMX | 21 | 15 |
| Bridge 1 | 23 | D8 | 560 | Arylex | 5 | ZEAMX | 21 | 5 |
| Bridge 1 | 24 | D8 | 1120 | Arylex | 5 | ZEAMX | 21 | 5 |
| Bridge 1 | 24 | D8 | 1120 | Arylex | 5 | ZEAMX | 21 | 5 |
| Bridge 1 | 24 | D8 | 1120 | Arylex | 5 | ZEAMX | 21 | 20 |
| Bridge 1 | 25 | D8 | 560 | Mesotrione | 150 | ZEAMX | 21 | 5 |
| Bridge 1 | 25 | D8 | 560 | Mesotrione | 150 | ZEAMX | 21 | 10 |
| Bridge 1 | 25 | D8 | 560 | Mesotrione | 150 | ZEAMX | 21 | 5 |
| Bridge 1 | 26 | D8 | 1120 | Mesotrione | 150 | ZEAMX | 21 | 35 |
| Bridge 1 | 26 | D8 | 1120 | Mesotrione | 150 | ZEAMX | 21 | 10 |
| Bridge 1 | 26 | D8 | 1120 | Mesotrione | 150 | ZEAMX | 21 | 10 |
| Bridge 1 | 27 | D8 | 560 | Glyphosate | 1120 | ZEAMX | 21 | 70 |
| Bridge 1 | 27 | D8 | 560 | Glyphosate | 1120 | ZEAMX | 21 | 65 |
| Bridge 1 | 27 | D8 | 560 | Glyphosate | 1120 | ZEAMX | 21 | 70 |
| Bridge 1 | 28 | D8 | 1120 | Glyphosate | 1120 | ZEAMX | 21 | 70 |
| Bridge 1 | 28 | D8 | 1120 | Glyphosate | 1120 | ZEAMX | 21 | 70 |
| Bridge 1 | 28 | D8 | 1120 | Glyphosate | 1120 | ZEAMX | 21 | 65 |
| Bridge 1 | 29 | D8 | 560 | Glufosinate | 600 | ZEAMX | 21 | 30 |
| Bridge 1 | 29 | D8 | 560 | Glufosinate | 600 | ZEAMX | 21 | 30 |
| Bridge 1 | 29 | D8 | 560 | Glufosinate | 600 | ZEAM> | 21 | 15 |
| Bridge 1 | 30 | D8 | 1120 | Glufosinate | 600 | ZEAMX | 21 | 70 |
| Bridge 1 | 30 | D8 | 1120 | Glufosinate | 600 | ZEAMX | 21 | 40 |
| Bridge 1 | 30 | D8 | 1120 | Glufosinate | 600 | ZEAMX | 21 | 40 |
| Bridge 1 | 31 | D27 | 560 | none | 0 | ZEAMX | 21 | 5 |
| Bridge 1 | 31 | D27 | 560 | none | 0 | ZEAMX | 21 | 5 |
| Bridge 1 | 31 | D27 | 560 | none | 0 | ZEAMX | 21 | 5 |
| Bridge 1 | 32 | D27 | 1120 | none | 0 | ZEAMX | 21 | 10 |
| Bridge 1 | 32 | D27 | 1120 | none | 0 | ZEAMX | 21 | 15 |
| Bridge 1 | 32 | D27 | 1120 | none | 0 | ZEAMX | 21 | 5 |
| Bridge 1 | 33 | D27 | 560 | diclosulam | 35 | ZEAMX | 21 | 65 |
| Bridge 1 | 33 | D27 | 560 | diclosulam | 35 | ZEAMX | 21 | 65 |
| Bridge 1 | 33 | D27 | 560 | diclosulam | 35 | ZEAMX | 21 | 70 |
| Bridge 1 | 34 | D27 | 1120 | diclosulam | 35 | ZEAMX | 21 | 65 |
| Bridge 1 | 34 | D27 | 1120 | diclosulam | 35 | ZEAMX | 21 | 60 |
| Bridge 1 | 34 | D27 | 1120 | diclosulam | 35 | ZEAMX | 21 | 65 |
| Bridge 1 | 35 | D27 | 560 | Arylex | 5 | ZEAMX | 21 | 25 |
| Bridge 1 | 35 | D27 | 560 | Arylex | 5 | ZEAMX | 21 | 5 |
| Bridge 1 | 35 | D27 | 560 | Arvlex | 5 | ZEAMX | 21 | 5 |
| Bridge 1 | 36 | D27 | 1120 | Arylex | 5 | ZEAMX | 21 | 10 |
| Bridge 1 | 36 | D27 | 1120 | Arylex | 5 | ZEAMX | 21 | 15 |
| Bridge 1 | 36 | D27 | 1120 | Arylex | 5 | ZEAMX | 21 | 10 |
| Bridge 1 | 37 | D27 | 560 | Mesotrione | 150 | ZEAMX | 21 | 5 |
| Bridge 1 | 37 | D27 | 560 | Mesotrione | 150 | ZEAMX | 21 | 5 |
| Bridge 1 | 37 | D27 | 560 | Mesotrione | 150 | ZEAMX | 21 | 10 |
| Bridge 1 | 38 | D27 | 1120 | Mesotrione | 150 | ZEAMX | 21 | 5 |
| Bridge 1 | 38 | D27 | 1120 | Mesotrione | 150 | ZEAMX | 21 | 10 |
| Bridge 1 | 38 | D27 | 1120 | Mesotrione | 150 | ZEAMX | 21 | 15 |
| Bridge 1 | 39 | D27 | 560 | Glyphosate | 1120 | ZEAMX | 21 | 70 |
| Bridge 1 | 39 | D27 | 560 | Glyphosate | 1120 | ZEAMX | 21 | 65 |
| Bridge 1 | 39 | D27 | 560 | Glyphosate | 1120 | ZEAMX | 21 | 70 |
| Bridge 1 | 40 | D27 | 1120 | Glyphosate | 1120 | ZEAMX | 21 | 70 |
| Bridge 1 | 40 | D27 | 1120 | Glyphosate | 1120 | ZEAMX | 21 | 99 |
| Bridge 1 | 40 | D27 | 1120 | Glyphosate | 1120 | ZEAMX | 21 | 70 |
| Bridge 1 | 41 | D27 | 560 | Glufosinate | 600 | ZEAMX | 21 | 15 |
| Bridge 1 | 41 | D27 | 560 | Glufosinate | 600 | ZEAMX | 21 | 5 |
| Bridge 1 | 41 | D27 | 560 | Glufosinate | 600 | ZEAMX | 21 | 5 |
| Bridge 1 | 42 | D27 | 1120 | Glufosinate | 600 | ZEAMX | 21 | 40 |
| Bridge 1 | 42 | D27 | 1120 | Glufosinate | 600 | ZEAMX | 21 | 35 |
| Bridge 1 | 42 | D2 | 1120 | Glufosinate | 600 | ZEAMX | 21 | 5 |
| Bridge 1 | 43 | B2 | 1120 | none | | ZEAMX | 21 | 35 |
| Bridge 1 | 43 | B2 | 1120 | none | | ZEAMX | 21 | 5 |
| Bridge 1 | 43 | B2 | 1120 | none | | ZEAMX | 21 | 5 |
| Bridge 1 | 44 | B2 | 1120 | diclosulam | 35 | ZEAMX | 21 | 50 |
| Bridge 1 | 44 | B2 | 1120 | diclosulam | 35 | ZEAMX | 21 | 65 |
| Bridge 1 | 44 | B2 | 1120 | diclosulam | 35 | ZEAMX | 21 | 70 |
| Bridge 1 | 45 | B2 | 1120 | Arylex | 5 | ZEAMX | 21 | 5 |
| Bridge 1 | 45 | B2 | 1120 | Arylex | 5 | ZEAMX | 21 | 5 |
| Bridge 1 | 45 | B2 | 1120 | Aryle | 5 | ZEAMX | 21 | 10 |
| Bridge 1 | 46 | B2 | 1120 | Mesotrione | 150 | ZEAMX | 21 | 15 |
| Bridge 1 | 46 | B2 | 1120 | Mesotrione | 150 | ZEAMX | 21 | 5 |
| Bridge 1 | 46 | B2 | 1120 | Mesotrione | 150 | ZEAMX | 21 | 10 |
| Bridge 1 | 47 | B2 | 1120 | Glyphosate | 1120 | ZEAMX | 21 | 75 |
| Bridge 1 | 47 | B2 | 1120 | Glyphosate | 1120 | ZEAMX | 21 | 70 |
| Bridge 1 | 47 | B2 | 1120 | Glyphosate | 1120 | ZEAMX | 21 | 95 |
| Bridge 1 | 48 | B2 | 1120 | Glufosinate | 600 | ZEAMX | 21 | 50 |
| Bridge 1 | 48 | B2 | 1120 | Glufosinate | 600 | ZEAMX | 21 | 5 |
| Bridge 1 | 48 | B2 | 1120 | Glufosinate | 600 | ZEAMX | 21 | 65 |
| Bridge 2 | 1 | Solv. Ck | 0 | 0 | 0 | ABUTH | 21 | 0 |
| | | untreated | | | | | | |
| Bridge 2 | 1 | Solv. Ck | 0 | 0 | 0 | ABUTH | 21 | 0 |
| | | untreated | | | | | | |
| Bridge 2 | 1 | Solv. Ck | 0 | 0 | 0 | ABUTH | 21 | 0 |
| | | untreated | | | | | | |
| Bridge 2 | 2 | B2 | 375 | none | | ABUTH | 21 | 100 |
| Bridge 2 | 2 | B2 | 375 | none | | ABUTH | 21 | 95 |
| Bridge 2 | 2 | B2 | 375 | none | | ABUTH | 21 | 100 |
| Bridge 2 | 3 | B2 | 560 | none | | ABUTH | 21 | 100 |
| Bridge 2 | 3 | B2 | 560 | none | | ABUTH | 21 | 100 |
| Bridge 2 | 3 | B2 | 560 | none | | ABUTH | 21 | 95 |
| Bridge 2 | 4 | B2 | 1120 | none | | ABUTH | 21 | 100 |
| Bridge 2 | 4 | B2 | 1120 | none | | ABUTH | 21 | 100 |
| Bridge 2 | 4 | B2 | 1120 | none | | ABUTH | 21 | 99 |
| Bridge 2 | 5 | B2 | 0 | Trifludimoxazin | 12.5 | ABUTH | 21 | 100 |
| Bridge 2 | 5 | B2 | 0 | Trifludimoxazin | 12.5 | ABUTH | 21 | 100 |
| Bridge 2 | 5 | B2 | 0 | Trifludimoxazin | 12.5 | ABUTH | 21 | 100 |
| Bridge 2 | 6 | B2 | 375 | Trifludimoxazin | 12.5 | ABUTH | 21 | 99 |
| Bridge 2 | 6 | B2 | 375 | Trifludimoxazin | 12.5 | ABUTH | 21 | 90 |
| Bridge 2 | 6 | B2 | 375 | Trifludimoxazin | 12.5 | ABUTH | 21 | 100 |
| Bridge 2 | 7 | B2 | 560 | Trifludimoxazin | 12.5 | ABUTH | 21 | 99 |
| Bridge 2 | 7 | B2 | 560 | Trifludimoxazin | 12.5 | ABUTH | 21 | 99 |
| Bridge 2 | 7 | B2 | 560 | Trifludimoxazin | 12.5 | ABUTH | 21 | 100 |
| Bridge 2 | 8 | B2 | 1120 | Trifludimoxazin | 12.5 | ABUTH | 21 | 100 |
| Bridge 2 | 8 | B2 | 1120 | Trifludimoxazin | 12.5 | ABUTH | 21 | 100 |
| Bridge 2 | 8 | B2 | 1120 | Trifludimoxazin | 12.5 | ABUTH | 21 | 100 |
| Bridge 2 | 9 | B2 | 0 | Trifludimoxazin | 25 | ABUTH | 21 | 100 |
| Bridge 2 | 9 | B2 | 0 | Trifludimoxazin | 25 | ABUTH | 21 | 100 |
| Bridge 2 | 9 | B2 | 0 | Trifludimoxazin | 25 | ABUTH | 21 | 100 |
| Bridge 2 | 10 | B2 | 375 | Trifludimoxazin | 25 | ABUTH | 21 | 100 |
| Bridge 2 | 10 | B2 | 375 | Trifludimoxazin | 25 | ABUTH | 21 | 100 |
| Bridge 2 | 10 | B2 | 375 | Trifludimoxazin | 25 | ABUTH | 21 | 100 |
| Bridge 2 | 11 | B2 | 560 | Trifludimoxazin | 25 | ABUTH | 21 | 100 |
| Bridge 2 | 11 | B2 | 560 | Trifludimoxazin | 25 | ABUTH | 21 | 100 |
| Bridge 2 | 11 | B2 | 560 | Trifludimovazin | 25 | ABUTH | 21 | 100 |
| Bridge 2 | 12 | B2 | 1120 | Trifludimoxazin | 25 | ABUTH | 21 | 100 |
| Bridge 2 | 12 | B2 | 1120 | Trifludimoxazin | 25 | ABUTH | 21 | 100 |
| Bridge 2 | 12 | B2 | 1120 | Trifludimoxazin | 25 | ABUTH | 21 | 100 |
| Bridge 2 | 13 | B2 | 0 | Trifludimoxazin | 50 | ABUTH | 21 | 100 |
| Bridge 2 | 13 | B2 | 0 | Trifludimoxazin | 50 | ABUTH | 21 | 100 |
| Bridge 2 | 13 | B2 | 0 | Trifludimoxazin | 50 | ABUTH | 21 | 100 |
| Bridge 2 | 14 | B2 | 375 | Trifludimoxazin | 50 | ABUTH | 21 | 100 |
| Bridge 2 | 14 | B2 | 375 | Trifludimoxazin | 50 | ABUTH | 21 | 100 |
| Bridge 2 | 14 | B2 | 375 | Trifludimoxazin | 50 | ABUTH | 21 | 100 |
| Bridge 2 | 15 | B2 | 560 | Trifludimoxazin | 50 | ABUTH | 21 | 100 |
| Bridge 2 | 15 | B2 | 560 | Trifludimoxazin | 50 | ABUTH | 21 | 100 |
| Bridge 2 | 15 | B2 | 560 | Trifludimoxazin | 50 | ABUTH | 21 | 100 |
| Bridge 2 | 16 | B2 | 1120 | Trifludimoxazin | 50 | ABUTH | 21 | 100 |
| Bridge 2 | 16 | B2 | 1120 | Trifludimoxazin | 50 | ABUTH | 21 | 100 |
| Bridge 2 | 16 | B2 | 1120 | Trifludimoxazin | 50 | ABUTH | 21 | 100 |
| Bridge 2 | 17 | D10 | 560 | none | 0 | ABUTH | 21 | 100 |
| Bridge 2 | 17 | D10 | 560 | none | 0 | ABUTH | 21 | 100 |
| Bridge 2 | 17 | D10 | 560 | none | 0 | ABUTH | 21 | 99 |
| Bridge 2 | 18 | D10 | 1120 | none | 0 | ABUTH | 21 | 100 |
| Bridge 2 | 18 | D10 | 1120 | none | 0 | ABUTH | 21 | 100 |
| Bridge 2 | 18 | D10 | 1120 | none | 0 | ABUTH | 21 | 99 |
| Bridge 2 | 19 | D10 | 560 | Trifludimoxazin | 12.5 | ABUTH | 21 | 100 |
| Bridge 2 | 19 | D10 | 560 | Trifludimoxazin | 12.5 | ABUTH | 21 | 100 |
| Bridge 2 | 19 | D10 | 560 | Trifludimoxazin | 12.5 | ABUTH | 21 | 100 |
| Bridge 2 | 20 | D10 | 1120 | Trifludimoxazin | 12.5 | ABUTH | 21 | 99 |
| Bridge 2 | 20 | D10 | 1120 | Trifludimoxazin | 12.5 | ABUTH | 21 | 100 |
| Bridge 2 | 20 | D10 | 1120 | Trifludimoxazin | 12.5 | ABUTH | 21 | 100 |
| Bridge 2 | 21 | D10 | 560 | Trifludimoxazin | 50 | ABUTH | 21 | 100 |
| Bridge 2 | 21 | D10 | 560 | Trifludimoxazin | 50 | ABUTH | 21 | 100 |
| Bridge 2 | 21 | D10 | 560 | Trifludimoxazin | 50 | ABUTH | 21 | 100 |
| Bridge 2 | 22 | D10 | 1120 | Trifludimoxazin | 50 | ABUTH | 21 | 100 |
| Bridge 2 | 22 | D10 | 1120 | Trifludimoxazin | 50 | ABUTH | 21 | 100 |
| Bridge 2 | 22 | D10 | 1120 | Trifludimoxazin | 50 | ABUTH | 21 | 100 |
| Bridge 2 | 23 | D10 | 560 | Saflufenacil | 25 | ABUTH | 21 | 100 |
| Bridge 2 | 23 | D10 | 560 | Saflufenacil | 25 | ABUTH | 21 | 100 |
| Bridge 2 | 23 | D10 | 560 | Saflufenacil | 25 | ABUTH | 21 | 100 |
| Bridge 2 | 24 | D10 | 1120 | Saflufenacil | 25 | ABUTH | 21 | 100 |
| Bridge 2 | 24 | D10 | 1120 | Saflufenacil | 25 | ABUTH | 21 | 100 |
| Bridge 2 | 24 | D10 | 1120 | Saflufenacil | 25 | ABUTH | 21 | 100 |
| Bridge 2 | 25 | D10 | 560 | Saflufenacil | 75 | ABUTH | 21 | 100 |
| Bridge 2 | 25 | D10 | 560 | Saflufenacil | 75 | ABUTH | 21 | 100 |
| Bridge 2 | 25 | D10 | 560 | Saflufenacil | 75 | ABUTH | 21 | 100 |
| Bridge 2 | 26 | D10 | 1120 | Saflufenacil | 75 | ABUTH | 21 | 100 |
| Bridge 2 | 26 | D10 | 1120 | Saflufenacil | 75 | ABUTH | 21 | 100 |
| Bridge 2 | 26 | D10 | 1120 | Saflufenacil | 75 | ABUTH | 21 | 100 |
| Bridge 2 | 27 | D8 | 560 | none | 0 | ABUTH | 21 | 100 |
| Bridge 2 | 27 | D8 | 560 | none | 0 | ABUTH | 21 | 99 |
| Bridge 2 | 27 | D8 | 560 | none | 0 | ABUTH | 21 | 100 |
| Bridge 2 | 28 | D8 | 1120 | none | 0 | ABUTH | 21 | 100 |
| Bridge 2 | 28 | D8 | 1120 | none | 0 | ABUTH | 21 | 100 |
| Bridge 2 | 28 | D8 | 1120 | none | 0 | ABUTH | 21 | 100 |
| Bridge 2 | 29 | D8 | 560 | Trifludimoxazin | 12.5 | ABUTH | 21 | 100 |
| Bridge 2 | 29 | D8 | 560 | Trifludimoxazin | 12.5 | ABUTH | 21 | 100 |
| Bridge 2 | 29 | D8 | 560 | Trifludimoxazin | 12.5 | ABUTH | 21 | 99 |
| Bridge 2 | 30 | D8 | 1120 | Trifludimoxazin | 12.5 | ABUTH | 21 | 100 |
| Bridge 2 | 30 | D8 | 1120 | Trifludimoxazin | 12.5 | ABUTH | 21 | 100 |
| Bridge 2 | 30 | D8 | 1120 | Trifludimoxazin | 12.5 | ABUTH | 21 | 100 |
| Bridge 2 | 31 | D8 | 560 | Trifludimoxazin | 50 | ABUTH | 21 | 100 |
| Bridge 2 | 31 | D8 | 560 | Trifludimoxazin | 50 | ABUTH | 21 | 100 |
| Bridge 2 | 31 | D8 | 560 | Trifludimoxazin | 50 | ABUTH | 21 | 100 |
| Bridge 2 | 32 | D8 | 1120 | Trifludimoxazin | 50 | ABUTH | 21 | 100 |
| Bridge 2 | 32 | D8 | 1120 | Trifludimoxazin | 50 | ABUTH | 21 | 100 |
| Bridge 2 | 32 | D8 | 1120 | Trifludimoxazin | 50 | ABUTH | 21 | 100 |
| Bridge 2 | 33 | D8 | 560 | Saflufenacil | 25 | ABUTH | 21 | 100 |
| Bridge 2 | 33 | D8 | 560 | Saflufenacil | 25 | ABUTH | 21 | 100 |
| Bridge 2 | 33 | D8 | 560 | Saflufenacil | 25 | ABUTH | 21 | 100 |
| Bridge 2 | 34 | D8 | 1120 | Saflufenacil | 25 | ABUTH | 21 | 100 |
| Bridge 2 | 34 | D8 | 1120 | Saflufenacil | 25 | ABUTH | 21 | 100 |
| Bridge 2 | 34 | D8 | 1120 | Saflufenacil | 25 | ABUTH | 21 | 100 |
| Bridge 2 | 35 | D8 | 560 | Saflufenacil | 75 | ABUTH | 21 | 100 |
| Bridge 2 | 35 | D8 | 560 | Saflufenacil | 75 | ABUTH | 21 | 100 |
| Bridge 2 | 35 | D8 | 560 | Saflufenacil | 75 | ABUTH | 21 | 100 |
| Bridge 2 | 36 | D8 | 1120 | Saflufenacil | 75 | ABUTH | 21 | 100 |
| Bridge 2 | 36 | D8 | 1120 | Saflufenacil | 75 | ABUTH | 21 | 100 |
| Bridge 2 | 36 | D8 | 1120 | Saflufenacil | 75 | ABUTH | 21 | 100 |
| Bridge 2 | 37 | D27 | 560 | none | 0 | ABUTH | 21 | 100 |
| Bridge 2 | 37 | D27 | 560 | none | 0 | ABUTH | 21 | 100 |
| Bridge 2 | 37 | D27 | 560 | none | 0 | ABUTH | 21 | 100 |
| Bridge 2 | 38 | D27 | 1120 | none | 0 | ABUTH | 21 | 100 |
| Bridge 2 | 38 | D27 | 1120 | none | 0 | ABUTH | 21 | 100 |
| Bridge 2 | 38 | D27 | 1120 | none | 0 | ABUTH | 21 | 100 |
| Bridge 2 | 39 | D27 | 560 | Trifludimoxazin | 12.5 | ABUTH | 21 | 100 |
| Bridge 2 | 39 | D27 | 560 | Trifludimoxazin | 12.5 | ABUTH | 21 | 100 |
| Bridge 2 | 39 | D27 | 560 | Trifludimoxazin | 12.5 | ABUTH | 21 | 100 |
| Bridge 2 | 40 | D27 | 1120 | Trifludimoxazin | 12.5 | ABUTH | 21 | 100 |
| Bridge 2 | 40 | D27 | 1120 | Trifludimoxazin | 12.5 | ABUTH | 21 | 100 |
| Bridge 2 | 40 | D27 | 1120 | Trifludimoxazin | 12.5 | ABUTH | 21 | 100 |
| Bridge 2 | 41 | D27 | 560 | Trifludimoxazin | 50 | ABUTH | 21 | 100 |
| Bridge 2 | 41 | D27 | 560 | Trifludimoxazin | 50 | ABUTH | 21 | 100 |
| Bridge 2 | 41 | D27 | 560 | Trifludimoxazin | 50 | ABUTH | 21 | 100 |
| Bridge 2 | 42 | D27 | 1120 | Trifludimoxazin | 50 | ABUTH | 21 | 100 |
| Bridge 2 | 42 | D27 | 1120 | Trifludimoxazin | 50 | ABUTH | 21 | 100 |
| Bridge 2 | 42 | D27 | 1120 | Trifludimoxazin | 50 | ABUTH | 21 | 100 |
| Bridge 2 | 43 | D27 | 560 | Saflufenacil | 25 | ABUTH | 21 | 100 |
| Bridge 2 | 43 | D27 | 560 | Saflufenacil | 25 | ABUTH | 21 | 100 |
| Bridge 2 | 43 | D27 | 560 | Saflufenacil | 25 | ABUTH | 21 | 100 |
| Bridge 2 | 44 | D27 | 1120 | Saflufenacil | 25 | ABUTH | 21 | 100 |
| Bridge 2 | 44 | D27 | 1120 | Saflufenacil | 25 | ABUTH | 21 | 100 |
| Bridge 2 | 44 | D27 | 1120 | Saflufenacil | 25 | ABUTH | 21 | 100 |
| Bridge 2 | 45 | D27 | 560 | Saflufenacil | 75 | ABUTH | 21 | 100 |
| Bridge 2 | 45 | D27 | 560 | Saflufenacil | 75 | ABUTH | 21 | 100 |
| Bridge 2 | 45 | D27 | 560 | Saflufenacil | 75 | ABUTH | 21 | 100 |
| Bridge 2 | 46 | D27 | 1120 | Saflufenacil | 75 | ABUTH | 21 | 100 |
| Bridge 2 | 46 | D27 | 1120 | Saflufenacil | 75 | ABUTH | 21 | 100 |
| Bridge 2 | 46 | D27 | 1120 | Saflufenacil | 75 | ABUTH | 21 | 100 |
| Bridge 2 | 1 | Solv. Ck | 0 | 0 | 0 | AMATA | 21 | 0 |
| | | untreated | | | | | | |
| Bridge 2 | 1 | Solv. Ck | 0 | 0 | 0 | AMATA | 21 | 0 |
| | | untreated | | | | | | |
| Bridge 2 | 1 | Solv. Ck | 0 | 0 | 0 | AMATA | 121 | 0 |
| | | untreated | | | | | | |
| Bridge 2 | 2 | B2 | 375 | none | | AMATA | 21 | 100 |
| Bridge 2 | 2 | B2 | 375 | none | | AMATA | 21 | 100 |
| Bridge 2 | 2 | B2 | 375 | none | | AMATA | 21 | 70 |
| Bridge 2 | 3 | B2 | 560 | none | | AMATA | 21 | 100 |
| Bridge 2 | 3 | B2 | 560 | none | | AMATA | 21 | 100 |
| Bridge 2 | 3 | B2 | 560 | none | | AMATA | 21 | 100 |
| Bridge 2 | 4 | B2 | 1120 | none | | AMATA | 21 | 100 |
| Bridge 2 | 4 | B2 | 1120 | none | | AMATA | 21 | 100 |
| Bridge 2 | 4 | B2 | 1120 | none | | AMATA | 21 | 90 |
| Bridge 2 | 5 | B2 | 0 | Trifludimoxazin | 12.5 | AMATA | 21 | 100 |
| Bridge 2 | 5 | B2 | 0 | Trifludimoxazin | 12.5 | AMATA | 21 | 100 |
| Bridge 2 | 5 | B2 | 0 | Trifludimoxazin | 12.5 | AMATA | 21 | 100 |
| Bridge 2 | 6 | B2 | 375 | Trifludimoxazin | 12.5 | AMATA | 21 | 100 |
| Bridge 2 | 6 | B2 | 375 | Trifludimoxazin | 12.5 | AMATA | 21 | 100 |
| Bridge 2 | 6 | B2 | 375 | Trifludimoxazin | 12.5 | AMATA | 21 | 100 |
| Bridge 2 | 7 | B2 | 560 | Trifludimoxazin | 12.5 | AMATA | 21 | 100 |
| Bridge 2 | 7 | B2 | 560 | Trifludimoxazin | 12.5 | AMATA | 21 | 100 |
| Bridge 2 | 7 | B2 | 560 | Trifludimoxazin | 12.5 | AMATA | 21 | 100 |
| Bridge 2 | 8 | B2 | 1120 | Trifludimoxazin | 12.5 | AMATA | 21 | 100 |
| Bridge 2 | 8 | B2 | 1120 | Trifludimoxazin | 12.5 | AMATA | 21 | 100 |
| Bridge 2 | 8 | B2 | 1120 | Trifludimoxazin | 12.5 | AMATA | 21 | 100 |
| Bridge 2 | 9 | B2 | 0 | Trifludimoxazin | 25 | AMATA | 21 | 100 |
| Bridge 2 | 9 | B2 | 0 | Trifludimoxazin | 25 | AMATA | 21 | 100 |
| Bridge 2 | 9 | B2 | 0 | Trifludimoxazin | 25 | AMATA | 21 | 100 |
| Bridge 2 | 10 | B2 | 375 | Trifludimoxazin | 25 | AMATA | 21 | 100 |
| Bridge 2 | 10 | B2 | 375 | Trifludimoxazin | 25 | AMATA | 21 | 100 |
| Bridge 2 | 10 | B2 | 375 | Trifludimoxazin | 25 | AMATA | 21 | 100 |
| Bridge 2 | 11 | B2 | 560 | Trifludimoxazin | 25 | AMATA | 21 | 100 |
| Bridge 2 | 11 | B2 | 560 | Trifludimoxazin | 25 | AMATA | 21 | 100 |
| Bridge 2 | 11 | B2 | 560 | Trifludimoxazin | 25 | AMATA | 21 | 100 |
| Bridge 2 | 12 | B2 | 1120 | Trifludimoxazin | 25 | AMATA | 21 | 100 |
| Bridge 2 | 12 | B2 | 1120 | Trifludimoxazin | 25 | AMATA | 21 | 100 |
| Bridge 2 | 12 | B2 | 1120 | Trifludimoxazin | 25 | AMATA | 21 | 100 |
| Bridge 2 | 13 | B2 | 0 | Trifludimoxazin | 50 | AMATA | 21 | 100 |
| Bridge 2 | 13 | B2 | 0 | Trifludimoxazin | 50 | AMATA | 21 | 100 |
| Bridge 2 | 13 | B2 | 0 | Trifludimoxazin | 50 | AMATA | 21 | 100 |
| Bridge 2 | 14 | B2 | 375 | Trifludimoxazin | 50 | AMATA | 21 | 100 |
| Bridge 2 | 14 | B2 | 375 | Trifludimoxazin | 50 | AMATA | 21 | 100 |
| Bridge 2 | 14 | B2 | 375 | Trifludimoxazin | 50 | AMATA | 21 | 100 |
| Bridge 2 | 15 | B2 | 560 | Trifludimoxazin | 50 | AMATA | 21 | 100 |
| Bridge 2 | 15 | B2 | 560 | Trifludimoxazin | 50 | AMATA | 21 | 100 |
| Bridge 2 | 15 | B2 | 560 | Trifludimoxazin | 50 | AMATA | 21 | 100 |
| Bridge 2 | 16 | B2 | 1120 | Trifludimoxazin | 50 | AMATA | 21 | 100 |
| Bridge 2 | 16 | B2 | 1120 | Trifludimoxazin | 50 | AMATA | 21 | 100 |
| Bridge 2 | 16 | B2 | 1120 | Trifludimoxazin | 50 | AMATA | 21 | 100 |
| Bridge 2 | 17 | D10 | 560 | none | 0 | AMATA | 21 | 100 |
| Bridge 2 | 17 | D10 | 560 | none | 0 | AMATA | 21 | 100 |
| Bridge 2 | 17 | D10 | 560 | none | 0 | AMATA | 21 | 85 |
| Bridge 2 | 18 | D10 | 1120 | none | 0 | AMATA | 21 | 100 |
| Bridge 2 | 18 | D10 | 1120 | none | 0 | AMATA | 21 | 100 |
| Bridge 2 | 18 | D10 | 1120 | none | 0 | AMATA | 21 | 100 |
| Bridge 2 | 19 | D10 | 560 | Trifludimoxazin | 12.5 | AMATA | 21 | 100 |
| Bridge 2 | 19 | D10 | 560 | Trifludimoxazin | 12.5 | AMATA | 21 | 100 |
| Bridge 2 | 19 | D10 | 560 | Trifludimoxazin | 12.5 | AMATA | 21 | 100 |
| Bridge 2 | 20 | D10 | 1120 | Trifludimoxazin | 12.5 | AMATA | 21 | 100 |
| Bridge 2 | 20 | D10 | 1120 | Trifludimoxazin | 12.5 | AMATA | 21 | 100 |
| Bridge 2 | 20 | D10 | 1120 | Trifludimoxazin | 12.5 | AMATA | 21 | 100 |
| Bridge 2 | 21 | D10 | 560 | Trifludimoxazin | 50 | AMATA | 21 | 100 |
| Bridge 2 | 21 | D10 | 560 | Trifludimoxazin | 50 | AMATA | 21 | 100 |
| Bridge 2 | 21 | D10 | 560 | Trifludimoxazin | 50 | AMATA | 21 | 100 |
| Bridge 2 | 22 | D10 | 1120 | Trifludimoxazin | 50 | AMATA | 21 | 100 |
| Bridge 2 | 22 | D10 | 1120 | Trifludimoxazin | 50 | AMATA | 21 | 100 |
| Bridge 2 | 22 | D10 | 1120 | Trifludimoxazin | 50 | AMATA | 21 | 100 |
| Bridge 2 | 23 | D10 | 560 | Saflufenacil | 25 | AMATA | 21 | 100 |
| Bridge 2 | 23 | D10 | 560 | Saflufenacil | 25 | AMATA | 21 | 100 |
| Bridge 2 | 23 | D10 | 560 | Saflufenacil | 25 | AMATA | 21 | 100 |
| Bridge 2 | 24 | D10 | 1120 | Saflufenacil | 25 | AMATA | 21 | 100 |
| Bridge 2 | 24 | D10 | 1120 | Saflufenacil | 25 | AMATA | 21 | 100 |
| Bridge 2 | 24 | D10 | 1120 | Saflufenacil | 25 | AMATA | 21 | 100 |
| Bridge 2 | 25 | D10 | 560 | Saflufenacil | 75 | AMATA | 21 | 100 |
| Bridge 2 | 25 | D10 | 560 | Saflufenacil | 75 | AMATA | 21 | 100 |
| Bridge 2 | 25 | D10 | 560 | Saflufenacil | 75 | AMATA | 21 | 100 |
| Bridge 2 | 26 | D10 | 1120 | Saflufenacil | 75 | AMATA | 21 | 100 |
| Bridge 2 | 26 | D10 | 1120 | Saflufenacil | 75 | AMATA | 21 | 100 |
| Bridge 2 | 26 | D10 | 1120 | Saflufenacil | 75 | AMATA | 21 | 100 |
| Bridge 2 | 27 | D8 | 560 | none | 0 | AMATA | 21 | 100 |
| Bridge 2 | 27 | D8 | 560 | none | 0 | AMATA | 21 | 100 |
| Bridge 2 | 27 | D8 | 560 | none | 0 | AMATA | 21 | 90 |
| Bridge 2 | 28 | D8 | 1120 | none | 0 | AMATA | 21 | 100 |
| Bridge 2 | 28 | D8 | 1120 | none | 0 | AMATA | 21 | 100 |
| Bridge 2 | 28 | D8 | 1120 | none | 0 | AMATA | 21 | 100 |
| Bridge 2 | 29 | D8 | 560 | Trifludimoxazin | 12.5 | AMATA | 21 | 100 |
| Bridge 2 | 29 | D8 | 560 | Trifludimoxazin | 12.5 | AMATA | 21 | 100 |
| Bridge 2 | 29 | D8 | 560 | Trifludimoxazin | 12.5 | AMATA | 21 | 100 |
| Bridge 2 | 30 | D8 | 1120 | Trifludimoxazin | 12.5 | AMATA | 21 | 100 |
| Bridge 2 | 30 | D8 | 1120 | Trifludimoxazin | 12.5 | AMATA | 21 | 100 |
| Bridge 2 | 30 | D8 | 1120 | Trifludimoxazin | 12.5 | AMATA | 21 | 100 |
| Bridge 2 | 31 | D8 | 560 | Trifludimoxazin | 50 | AMATA | 21 | 100 |
| Bridge 2 | 31 | D8 | 560 | Trifludimoxazin | 50 | AMATA | 21 | 100 |
| Bridge 2 | 31 | D8 | 560 | Trifludimoxazin | 50 | AMATA | 21 | 100 |
| Bridge 2 | 32 | D8 | 1120 | Trifludimoxazin | 50 | AMATA | 21 | 100 |
| Bridge 2 | 32 | D8 | 1120 | Trifludimoxazin | 50 | AMATA | 21 | 100 |
| Bridge 2 | 32 | D8 | 1120 | Trifludimoxazin | 50 | AMATA | 21 | 100 |
| Bridge 2 | 33 | D8 | 560 | Saflufenacil | 25 | AMATA | 21 | 100 |
| Bridge 2 | 33 | D8 | 560 | Saflufenacil | 25 | AMATA | 21 | 100 |
| Bridge 2 | 33 | D8 | 560 | Saflufenacil | 25 | AMATA | 21 | 100 |
| Bridge 2 | 34 | D8 | 1120 | Saflufenacil | 25 | AMATA | 21 | 100 |
| Bridge 2 | 34 | D8 | 1120 | Saflufenacil | 25 | AMATA | 21 | 100 |
| Bridge 2 | 34 | D8 | 1120 | Saflufenacil | 25 | AMATA | 21 | 100 |
| Bridge 2 | 35 | D8 | 560 | Saflufenacil | 75 | AMATA | 21 | 100 |
| Bridge 2 | 35 | D8 | 560 | Saflufenacil | 75 | AMATA | 21 | 100 |
| Bridge 2 | 35 | D8 | 560 | Saflufenacil | 75 | AMATA | 21 | 100 |
| Bridge 2 | 36 | D8 | 1120 | Saflufenacil | 75 | AMATA | 21 | 100 |
| Bridge 2 | 36 | D8 | 1120 | Saflufenacil | 75 | AMATA | 21 | 100 |
| Bridge 2 | 36 | D8 | 1120 | Saflufenacil | 75 | AMATA | 21 | 100 |
| Bridge 2 | 37 | D27 | 560 | none | 0 | AMATA | 21 | 100 |
| Bridge 2 | 37 | D27 | 560 | none | 0 | AMATA | 21 | 100 |
| Bridge 2 | 37 | D27 | 560 | none | 0 | AMATA | 21 | 92 |
| Bridge 2 | 38 | D27 | 1120 | none | 0 | AMATA | 21 | 100 |
| Bridge 2 | 38 | D27 | 1120 | none | 0 | AMATA | 21 | 95 |
| Bridge 2 | 38 | D27 | 1120 | none | 0 | AMATA | 21 | 100 |
| Bridge 2 | 39 | D27 | 560 | Trifludimoxazin | 12.5 | AMATA | 21 | 100 |
| Bridge 2 | 39 | D27 | 560 | Trifludimoxazin | 12.5 | AMATA | 21 | 100 |
| Bridge 2 | 39 | D27 | $60 | Trifludimoxazin | 12.5 | AMATA | 21 | 100 |
| Bridge 2 | 40 | D27 | 1120 | Trifludimoxazin | 12.5 | AMATA | 21 | 100 |
| Bridge 2 | 40 | D27 | 1120 | Trifludimovazin | 12.5 | AMATA | 21 | 100 |
| Bridge 2 | 40 | D27 | 1120 | Trifludimoxazin | 12.5 | AMATA | 21 | 100 |
| Bridge 2 | 41 | D27 | 560 | Trifludimoxazin | 50 | AMATA | 21 | 100 |
| Bridge 2 | 41 | D27 | 560 | Trifludimoxazin | 50 | AMATA | 21 | 100 |
| Bridge 2 | 41 | D27 | 560 | Trifludimoxazin | 50 | AMATA | 21 | 100 |
| Bridge 2 | 42 | D27 | 1120 | Trifludimoxazin | 50 | AMATA | 21 | 100 |
| Bridge 2 | 42 | D27 | 1120 | Trifludimoxazin | 50 | AMATA | 21 | 100 |
| Bridge 2 | 42 | D27 | 1120 | Trifludimoxazin | 50 | AMATA | 21 | 95 |
| Bridge 2 | 43 | D27 | 560 | Saflufenacil | 25 | AMATA | 21 | 100 |
| Bridge 2 | 43 | D27 | 560 | Saflufenacil | 25 | AMATA | 21 | 100 |
| Bridge 2 | 43 | D27 | 560 | Saflufenacil | 25 | AMATA | 21 | 100 |
| Bridge 2 | 44 | D27 | 1120 | Saflufenacil | 25 | AMATA | 21 | 100 |
| Bridge 2 | 44 | D27 | 1120 | Saflufenacil | 25 | AMATA | 21 | 100 |
| Bridge 2 | 44 | D27 | 1120 | Saflufenacil | 25 | AMATA | 21 | 100 |
| Bridge 2 | 45 | D27 | 560 | Saflufenacil | 75 | AMATA | 21 | 100 |
| Bridge 2 | 45 | D27 | 560 | Saflufenacil | 75 | AMATA | 21 | 100 |
| Bridge 2 | 45 | D27 | 560 | Saflufenacil | 75 | AMATA | 21 | 100 |
| Bridge 2 | 46 | D27 | 1120 | Saflufenacil | 75 | AMATA | 21 | 100 |
| Bridge 2 | 46 | D27 | 1120 | Saflufenacil | 75 | AMATA | 21 | 100 |
| Bridge 2 | 46 | D27 | 1120 | Saflufenacil | 75 | AMATA | 21 | 100 |
| Bridge 2 | 1 | Solv. Ck | 0 | 0 | 0 | CHEAL | 21 | 0 |
| | | untreated | | | | | | |
| Bridge 2 | 1 | Solv. Ck | 0 | 0 | 0 | CHEAL | 21 | 0 |
| | | untreated | | | | | | |
| Bridge 2 | 1 | Solv. Ck | 0 | 0 | 0 | CHEAL | 21 | 0 |
| | | untreated | | | | | | |
| Bridge 2 | 2 | B2 | 375 | none | | CHEAL | 21 | 10 |
| Bridge 2 | 2 | B2 | 375 | none | | CHEAL | 21 | 95 |
| Bridge 2 | 2 | B2 | 375 | none | | CHEAL | 21 | 100 |
| Bridge 2 | 3 | B2 | 560 | none | | CHEAL | 21 | 100 |
| Bridge 2 | 3 | B2 | 560 | none | | CHEAL | 21 | 100 |
| Bridge 2 | 3 | B2 | 560 | none | | CHEAL | 21 | 100 |
| Bridge 2 | 4 | B2 | 1120 | none | | CHEAL | 21 | 100 |
| Bridge 2 | 4 | B2 | 1120 | none | | CHEAL | 21 | 100 |
| Bridge 2 | 4 | B2 | 1120 | none | | CHEAL | 21 | 100 |
| Bridge 2 | 5 | B2 | 0 | Trifludimoxazin | 12.5 | CHEAL | 21 | 100 |
| Bridge 2 | 5 | B2 | 0 | Trifludimoxazin | 12.5 | CHEAL | 21 | 100 |
| Bridge 2 | 5 | B2 | 0 | Trifludimoxazin | 12.5 | CHEAL | 21 | 100 |
| Bridge 2 | 6 | B2 | 375 | Trifludimoxazin | 12.5 | CHEAL | 21 | 100 |
| Bridge 2 | 6 | B2 | 375 | Trifludimoxazin | 12.5 | CHEAL | 21 | 100 |
| Bridge 2 | 6 | B2 | 375 | Trifludimoxazin | 12.5 | CHEAL | 21 | 100 |
| Bridge 2 | 7 | B2 | 560 | Trifludimoxazin | 12.5 | CHEAL | 21 | 100 |
| Bridge 2 | 7 | B2 | 560 | Trifludimoxazin | 12.5 | CHEAL | 21 | 100 |
| Bridge 2 | 7 | B2 | 560 | Trifludimoxazin | 12.5 | CHEAL | 21 | 100 |
| Bridge 2 | 8 | B2 | 1120 | Trifludimoxazin | 12.5 | CHEAL | 21 | 100 |
| Bridge 2 | 8 | B2 | 1120 | Trifludimoxazin | 12.5 | CHEAL | 21 | 100 |
| Bridge 2 | 8 | B2 | 1120 | Trifludimoxazin | 12.5 | CHEAL | 21 | 100 |
| Bridge 2 | 9 | B2 | 0 | Trifludimoxazin | 25 | CHEAL | 21 | 100 |
| Bridge 2 | 9 | B2 | 0 | Trifludimoxazin | 25 | CHEAL | 21 | 100 |
| Bridge 2 | 9 | B2 | 0 | Trifludimoxazin | 25 | CHEAL | 21 | 100 |
| Bridge 2 | 10 | B2 | 375 | Trifludimoxazin | 25 | CHEAL | 21 | 100 |
| Bridge 2 | 10 | B2 | 375 | Trifludimoxazin | 25 | CHEAL | 21 | 100 |
| Bridge 2 | 10 | B2 | 375 | Trifludimoxazin | 25 | CHEAL | 21 | 100 |
| Bridge 2 | 11 | B2 | 560 | Trifludimoxazin | 25 | CHEAL | 21 | 100 |
| Bridge 2 | 11 | B2 | 560 | Trifludimoxazin | 25 | CHEAL | 21 | 100 |
| Bridge 2 | 11 | B2 | 560 | Trifludimoxazin | 25 | CHEAL | 21 | 100 |
| Bridge 2 | 12 | B2 | 1120 | Trifludimoxazin | 25 | CHEAL | 21 | 100 |
| Bridge 2 | 12 | B2 | 1120 | Trifludimoxazin | 25 | CHEAL | 21 | 100 |
| Bridge 2 | 12 | B2 | 1120 | Trifludimoxazin | 25 | CHEAL | 21 | 100 |
| Bridge 2 | 13 | B2 | 0 | Trifludimoxazin | 50 | CHEAL | 21 | 100 |
| Bridge 2 | 13 | B2 | 0 | Trifludimoxazin | 50 | CHEAL | 21 | 100 |
| Bridge 2 | 13 | B2 | 0 | Trifludimovazin | 50 | CHEAL | 21 | 100 |
| Bridge 2 | 14 | B2 | 375 | Trifludimoxazin | 50 | CHEAL | 21 | 100 |
| Bridge 2 | 14 | B2 | 375 | Trifludimoxazin | 50 | CHEAL | 21 | 100 |
| Bridge 2 | 14 | B2 | 375 | Trifludimoxazin | 50 | CHEAL | 21 | 100 |
| Bridge 2 | 15 | B2 | 560 | Trifludimoxazin | 50 | CHEAL | 21 | 100 |
| Bridge 2 | 15 | B2 | 560 | Trifludimoxazin | 50 | CHEAL | 21 | 100 |
| Bridge 2 | 15 | B2 | 560 | Trifludimoxazin | 50 | CHEAL | 21 | 100 |
| Bridge 2 | 16 | B2 | 1120 | Trifludimoxazin | 50 | CHEAL | 21 | 100 |
| Bridge 2 | 16 | B2 | 1120 | Trifludimoxazin | 50 | CHEAL | 21 | 100 |
| Bridge 2 | 16 | B2 | 1120 | Trifludimoxazin | 50 | CHEAL | 21 | 100 |
| Bridge 2 | 17 | D10 | 560 | none | 0 | CHEAL | 21 | 95 |
| Bridge 2 | 17 | D10 | 560 | none | 0 | CHEAL | 21 | 100 |
| Bridge 2 | 17 | D10 | 560 | none | 0 | CHEAL | 21 | 100 |
| Bridge 2 | 18 | D10 | 1120 | none | 0 | CHEAL | 21 | 95 |
| Bridge 2 | 18 | D10 | 1120 | none | 0 | CHEAL | 21 | 92 |
| Bridge 2 | 18 | D10 | 1120 | none | 0 | CHEAL | 21 | 100 |
| Bridge 2 | 19 | D10 | 560 | Trifludimoxazin | 12.5 | CHEAL | 21 | 100 |
| Bridge 2 | 19 | D10 | 560 | Trifludimoxazin | 12.5 | CHEAL | 21 | 100 |
| Bridge 2 | 19 | D10 | 560 | Trifludimoxazin | 12.5 | CHEAL | 21 | 100 |
| Bridge 2 | 20 | D10 | 1120 | Trifludimoxazin | 12.5 | CHEAL | 21 | 100 |
| Bridge 2 | 20 | D10 | 1120 | Trifludimoxazin | 12.5 | CHEAL | 21 | 100 |
| Bridge 2 | 20 | D10 | 1120 | Trifludimoxazin | 12.5 | CHEAL | 21 | 100 |
| Bridge 2 | 21 | D10 | 560 | Trifludimoxazin | 50 | CHEAL | 21 | 100 |
| Bridge 2 | 21 | D10 | 560 | Trifludimoxazin | 50 | CHEAL | 21 | 100 |
| Bridge 2 | 21 | D10 | 560 | Trifludimovazin | 50 | CHEAL | 21 | 100 |
| Bridge 2 | 22 | D10 | 1120 | Trifludimoxazin | 50 | CHEAL | 21 | 100 |
| Bridge 2 | 22 | D10 | 1120 | Trifludimoxazin | 50 | CHEAL | 21 | 100 |
| Bridge 2 | 22 | D10 | 1120 | Trifludimoxazin | 50 | CHEAL | 21 | 100 |
| Bridge 2 | 23 | D10 | 560 | Saflufenacil | 25 | CHEAL | 21 | 100 |
| Bridge 2 | 23 | D10 | 560 | Saflufenacil | 25 | CHEAL | 21 | 100 |
| Bridge 2 | 23 | D10 | 560 | Saflufenacil | 25 | CHEAL | 21 | 100 |
| Bridge 2 | 24 | D10 | 1120 | Saflufenacil | 25 | CHEAL | 21 | 100 |
| Bridge 2 | 24 | D10 | 1120 | Saflufenacil | 25 | CHEAL | 21 | 100 |
| Bridge 2 | 24 | D10 | 1120 | Saflufenacil | 25 | CHEAL | 21 | 100 |
| Bridge 2 | 25 | D10 | 560 | Saflufenacil | 75 | CHEAL | 21 | 100 |
| Bridge 2 | 25 | D10 | 560 | Saflufenacil | 75 | CHEAL | 21 | 100 |
| Bridge 2 | 25 | D10 | 560 | Saflufenacil | 75 | CHEAL | 21 | 100 |
| Bridge 2 | 26 | D10 | 1120 | Saflufenacil | 75 | CHEAL | 21 | 100 |
| Bridge 2 | 26 | D10 | 1120 | Saflufenacil | 75 | CHEAL | 21 | 100 |
| Bridge 2 | 26 | D10 | 1120 | Saflufenacil | 75 | CHEAL | 21 | 100 |
| Bridge 2 | 27 | D8 | 560 | none | 0 | CHEAL | 21 | 98 |
| Bridge 2 | 27 | D8 | 560 | none | 0 | CHEAL | 21 | 95 |
| Bridge 2 | 27 | D8 | 560 | none | 0 | CHEAL | 21 | 100 |
| Bridge 2 | 28 | D8 | 1120 | none | 0 | CHEAL | 21 | 100 |
| Bridge 2 | 28 | D8 | 1120 | none | 0 | CHEAL | 21 | 100 |
| Bridge 2 | 28 | D8 | 1120 | none | 0 | CHEAL | 21 | 97 |
| Bridge 2 | 29 | D8 | 560 | Trifludimoxazin | 12.5 | CHEAL | 21 | 100 |
| Bridge 2 | 29 | D8 | 560 | Trifludimoxazin | 12.5 | CHEAL | 21 | 100 |
| Bridge 2 | 29 | D8 | 560 | Trifludimoxazin | 12.5 | CHEAL | 21 | 100 |
| Bridge 2 | 30 | D8 | 1120 | Trifludimoxazin | 12.5 | CHEAL | 21 | 100 |
| Bridge 2 | 30 | D8 | 1120 | Trifludimoxazin | 12.5 | CHEAL | 21 | 100 |
| Bridge 2 | 30 | D8 | 1120 | Trifludimoxazin | 12.5 | CHEAL | 21 | 100 |
| Bridge 2 | 31 | D8 | 560 | Trifludimoxazin | 50 | CHEAL | 21 | 100 |
| Bridge 2 | 31 | D8 | 560 | Trifludimoxazin | 50 | CHEAL | 21 | 100 |
| Bridge 2 | 31 | D8 | 560 | Trifludimoxazin | 50 | CHEAL | 21 | 100 |
| Bridge 2 | 32 | D8 | 1120 | Trifludimoxazin | 50 | CHEAL | 21 | 100 |
| Bridge 2 | 32 | D8 | 1120 | Trifludimoxazin | 50 | CHEAL | 21 | 100 |
| Bridge 2 | 32 | D8 | 1120 | Trifludimoxazin | 50 | CHEAL | 21 | 100 |
| Bridge 2 | 33 | D8 | 560 | Saflufenacil | 25 | CHEAL | 21 | 100 |
| Bridge 2 | 33 | D8 | 560 | Saflufenacil | 25 | CHEAL | 21 | 100 |
| Bridge 2 | 33 | D8 | 560 | Saflufenacil | 25 | CHEAL | 21 | 100 |
| Bridge 2 | 34 | D8 | 1120 | Saflufenacil | 25 | CHEAL | 21 | 100 |
| Bridge 2 | 34 | D8 | 1120 | Saflufenacil | 25 | CHEAL | 21 | 100 |
| Bridge 2 | 34 | D8 | 1120 | Saflufenacil | 25 | CHEAL | 21 | 100 |
| Bridge 2 | 35 | D8 | 560 | Saflufenacil | 75 | CHEAL | 21 | 100 |
| Bridge 2 | 35 | D8 | 560 | Saflufenacil | 75 | CHEAL | 21 | 100 |
| Bridge 2 | 35 | D8 | 560 | Saflufenacil | 75 | CHEAL | 21 | 100 |
| Bridge 2 | 36 | D8 | 1120 | Saflufenacil | 75 | CHEAL | 21 | 100 |
| Bridge 2 | 36 | D8 | 1120 | Saflufenacil | 75 | CHEAL | 21 | 100 |
| Bridge 2 | 36 | D8 | 1120 | Saflufenacil | 75 | CHEAL | 21 | 100 |
| Bridge 2 | 37 | D27 | 560 | none | 0 | CHEAL | 21 | 95 |
| Bridge 2 | 37 | D27 | 560 | none | 0 | CHEAL | 21 | 100 |
| Bridge 2 | 37 | D27 | 560 | none | 0 | CHEAL | 21 | 100 |
| Bridge 2 | 38 | D27 | 1120 | none | 0 | CHEAL | 21 | 98 |
| Bridge 2 | 38 | D27 | 1120 | none | 0 | CHEAL | 21 | 100 |
| Bridge 2 | 38 | D27 | 1120 | none | 0 | CHEAL | 21 | 100 |
| Bridge 2 | 39 | D27 | 560 | Trifludimoxazin | 12.5 | CHEAL | 21 | 100 |
| Bridge 2 | 39 | D27 | 560 | Trifludimoxazin | 12.5 | CHEAL | 21 | 100 |
| Bridge 2 | 39 | D27 | 560 | Trifludimoxazin | 12.5 | CHEAL | 21 | 100 |
| Bridge 2 | 40 | D27 | 1120 | Trifludimoxazin | 12.5 | CHEAL | 21 | 100 |
| Bridge 2 | 40 | D27 | 1120 | Trifludimoxazin | 12.5 | CHEAL | 21 | 100 |
| Bridge 2 | 40 | D27 | 1120 | Trifludimoxazin | 12.5 | CHEAL | 21 | 100 |
| Bridge 2 | 41 | D27 | 560 | Trifludimoxazin | 50 | CHEAL | 21 | 100 |
| Bridge 2 | 41 | D27 | 560 | Trifludimoxazin | 50 | CHEAL | 21 | 100 |
| Bridge 2 | 41 | D27 | 560 | Trifludimovazin | 50 | CHEAL | 21 | 100 |
| Bridge 2 | 42 | D27 | 1120 | Trifludimoxazin | 50 | CHEAL | 21 | 100 |
| Bridge 2 | 42 | D27 | 1120 | Trifludimoxazin | 50 | CHEAL | 21 | 100 |
| Bridge 2 | 42 | D27 | 1120 | Trifludimoxazin | 50 | CHEAL | 21 | 100 |
| Bridge 2 | 43 | D27 | 560 | Saflufenacil | 25 | CHEAL | 21 | 100 |
| Bridge 2 | 43 | D27 | 560 | Saflufenacil | 25 | CHEAL | 21 | 100 |
| Bridge 2 | 43 | D27 | 560 | Saflufenacil | 25 | CHEAL | 21 | 100 |
| Bridge 2 | 44 | D27 | 1120 | Saflufenacil | 25 | CHEAL | 21 | 100 |
| Bridge 2 | 44 | D27 | 1120 | Saflufenacil | 25 | CHEAL | 21 | 100 |
| Bridge 2 | 44 | D27 | 1120 | Saflufenacil | 25 | CHEAL | 21 | 100 |
| Bridge 2 | 45 | D27 | 560 | Saflufenacil | 75 | CHEAL | 21 | 100 |
| Bridge 2 | 45 | D27 | 560 | Saflufenacil | 75 | CHEAL | 21 | 100 |
| Bridge 2 | 45 | D2 | 560 | Saflufenacil | 75 | CHEAL | 21 | 100 |
| Bridge 2 | 46 | D27 | 1120 | Saflufenacil | 75 | CHEAL | 21 | 100 |
| Bridge 2 | 46 | D27 | 1120 | Saflufenacil | 75 | CHEAL | 21 | 100 |
| Bridge 2 | 46 | D27 | 1120 | Saflufenacil | 75 | CHEAL | 21 | 100 |
| Bridge 2 | 1 | Solv. Ck | | 0 | 0 | ERICA | 21 | 0 |
| | | untreated | | | | | | |
| Bridge 2 | 1 | Solv. Ck | 0 | 0 | 0 | ERICA | 21 | 0 |
| | | untreated | | | | | | |
| Bridge 2 | 2 | B2 | 375 | none | | ERICA | 21 | 97 |
| Bridge 2 | 2 | B2 | 375 | none | | ERICA | 21 | 99 |
| Bridge 2 | 3 | B2 | 560 | none | | ERICA | 21 | 99 |
| Bridge 2 | 3 | B2 | 560 | none | | ERICA | 21 | 100 |
| Bridge 2 | 4 | B2 | 1120 | none | | ERICA | 21 | 95 |
| Bridge 2 | 4 | B2 | 1120 | none | | ERICA | 21 | 95 |
| Bridge 2 | 5 | B2 | 0 | Trifludimoxazin | 12.5 | ERICA | 21 | 30 |
| Bridge 2 | 5 | B2 | 0 | Trifludimoxazin | 12.5 | ERICA | 21 | 10 |
| Bridge 2 | 6 | B2 | 375 | Trifludimoxazin | 12.5 | ERICA | 21 | 70 |
| Bridge 2 | 6 | B2 | 375 | Trifludimoxazin | 12.5 | ERICA | 21 | 70 |
| Bridge 2 | 7 | B2 | 560 | Trifludimoxazin | 12.5 | ERICA | 21 | 75 |
| Bridge 2 | 7 | B2 | 560 | Trifludimoxazin | 12.5 | ERICA | 21 | 95 |
| Bridge 2 | 8 | B2 | 1120 | Trifludimoxazin | 12.5 | ERICA | 21 | 90 |
| Bridge 2 | 8 | B2 | 1120 | Trifludimoxazin | 12.5 | ERICA | 21 | 70 |
| Bridge 2 | 9 | B2 | 0 | Trifludimoxazin | 25 | ERICA | 21 | 25 |
| Bridge 2 | 9 | B2 | 0 | Trifludimoxazin | 25 | ERICA | 21 | 30 |
| Bridge 2 | 10 | B2 | 375 | Trifludimoxazin | 25 | ERICA | 21 | 65 |
| Bridge 2 | 10 | B2 | 375 | Trifludimoxazin | 25 | ERICA | 21 | 65 |
| Bridge 2 | 11 | B2 | 560 | Trifludimoxazin | 25 | ERICA | 21 | 80 |
| Bridge 2 | 11 | B2 | 560 | Trifludimoxazin | 25 | ERICA | 21 | 60 |
| Bridge 2 | 12 | B2 | 1120 | Trifludimoxazin | 25 | ERICA | 21 | 60 |
| Bridge 2 | 12 | B2 | 1120 | Trifludimoxazin | 25 | ERICA | 21 | 65 |
| Bridge 2 | 13 | B2 | 0 | Trifludimoxazin | 50 | ERICA | 21 | 30 |
| Bridge 2 | 13 | B2 | 0 | Trifludimoxazin | 50 | ERICA | 21 | 25 |
| Bridge 2 | 14 | B2 | 375 | Trifludimoxazin | 50 | ERICA | 21 | 75 |
| Bridge 2 | 14 | B2 | 375 | Trifludimoxazin | 50 | ERICA | 21 | 65 |
| Bridge 2 | 15 | B2 | 560 | Trifludimoxazin | 50 | ERICA | 21 | 70 |
| Bridge 2 | 15 | B2 | 560 | Trifludimoxazin | 50 | ERICA | 21 | 75 |
| Bridge 2 | 16 | B2 | 1120 | Trifludimoxazin | 50 | ERICA | 21 | 80 |
| Bridge 2 | 16 | B2 | 1120 | Trifludimoxazin | 50 | ERICA | 21 | 80 |
| Bridge 2 | 17 | D10 | 560 | none | 0 | ERICA | 21 | 85 |
| Bridge 2 | 17 | D10 | 560 | none | 0 | ERICA | 21 | |
| Bridge 2 | 18 | D10 | 1120 | none | 0 | ERICA | 21 | 95 |
| Bridge 2 | 18 | D10 | 1120 | none | 0 | ERICA | 21 | 95 |
| Bridge 2 | 19 | D10 | 560 | Trifludimoxazin | 12.5 | ERICA | 21 | 95 |
| Bridge 2 | 19 | D10 | 560 | Trifludimoxazin | 12.5 | ERICA | 21 | 98 |
| Bridge 2 | 20 | D10 | 1120 | Trifludimoxazin | 12.5 | ERICA | 21 | 95 |
| Bridge 2 | 20 | D10 | 1120 | Trifludimoxazin | 12.5 | ERICA | 21 | 97 |
| Bridge 2 | 21 | D10 | 560 | Trifludimoxazin | 50 | ERICA | 21 | 90 |
| Bridge 2 | 21 | D10 | 560 | Trifludimoxazin | 50 | ERICA | 21 | 98 |
| Bridge 2 | 22 | D10 | 1120 | Trifludimoxazin | 50 | ERICA | 21 | 85 |
| Bridge 2 | 22 | D10 | 1120 | Trifludimoxazin | 50 | ERICA | 21 | 90 |
| Bridge 2 | 23 | D10 | 560 | Saflufenacil | 25 | ERICA | 21 | 100 |
| Bridge 2 | 23 | D10 | 560 | Saflufenacil | 25 | ERICA | 21 | 100 |
| Bridge 2 | 24 | D10 | 1120 | Saflufenacil | 25 | ERICA | 21 | 100 |
| Bridge 2 | 24 | D10 | 1120 | Saflufenacil | 25 | ERICA | 21 | 88 |
| Bridge 2 | 25 | D10 | 560 | Saflufenacil | 75 | ERICA | 21 | 100 |
| Bridge 2 | 25 | D10 | 560 | Saflufenacil | 75 | ERICA | 21 | 100 |
| Bridge 2 | 26 | D10 | 1120 | Saflufenacil | 75 | ERICA | 21 | 100 |
| Bridge 2 | 26 | D10 | 1120 | Saflufenacil | 75 | ERICA | 21 | 100 |
| Bridge 2 | 27 | D8 | 560 | none | 0 | ERICA | 21 | 95 |
| Bridge 2 | 27 | D8 | 560 | none | 0 | ERICA | 21 | |
| Bridge 2 | 28 | D8 | 1120 | none | 0 | ERICA | 21 | 97 |
| Bridge 2 | 28 | D8 | 1120 | none | 0 | ERICA | 21 | 100 |
| Bridge 2 | 29 | D8 | 560 | Trifludimoxazin | 12.5 | ERICA | 21 | 80 |
| Bridge 2 | 29 | D8 | 560 | Trifludimoxazin | 12.5 | ERICA | 21 | 97 |
| Bridge 2 | 30 | D8 | 1120 | Trifludimoxazin | 12.5 | ERICA | 21 | 85 |
| Bridge 2 | 30 | D8 | 1120 | Trifludimoxazin | 12.5 | ERICA | 21 | 92 |
| Bridge 2 | 31 | D8 | 560 | Trifludimoxazin | 50 | ERICA | 21 | 95 |
| Bridge 2 | 31 | D8 | 560 | Trifludimoxazin | 50 | ERICA | 21 | 90 |
| Bridge 2 | 32 | D8 | 1120 | Trifludimoxazin | 50 | ERICA | 21 | 60 |
| Bridge 2 | 32 | D8 | 1120 | Trifludimoxazin | 50 | ERICA | 21 | 80 |
| Bridge 2 | 33 | D8 | 560 | Saflufenacil | 25 | ERICA | 21 | 100 |
| Bridge 2 | 33 | D8 | 560 | Saflufenacil | 25 | ERICA | 21 | 100 |
| Bridge 2 | 34 | D8 | 1120 | Saflufenacil | 25 | ERICA | 21 | 100 |
| Bridge 2 | 34 | D8 | 1120 | Saflufenacil | 25 | ERICA | 21 | 95 |
| Bridge 2 | 35 | D8 | 560 | Saflufenacil | 75 | ERICA | 21 | 100 |
| Bridge 2 | 35 | D8 | 560 | Saflufenacil | 75 | ERICA | 21 | 100 |
| Bridge 2 | 36 | D8 | 1120 | Saflufenacil | 75 | ERICA | 21 | 100 |
| Bridge 2 | 36 | D8 | 1120 | Saflufenacil | 75 | ERICA | 21 | 100 |
| Bridge 2 | 37 | D27 | 560 | none | 0 | ERICA | 21 | 95 |
| Bridge 2 | 37 | D27 | 560 | none | 0 | ERICA | 21 | |
| Bridge 2 | 38 | D27 | 1120 | none | 0 | ERICA | 21 | 95 |
| Bridge 2 | 38 | D27 | 1120 | none | 0 | ERICA | 21 | 90 |
| Bridge 2 | 39 | D27 | 560 | Trifludimoxazin | 12.5 | ERICA | 21 | 80 |
| Bridge 2 | 39 | D27 | 560 | Trifludimoxazin | 12.5 | ERICA | 21 | 70 |
| Bridge 2 | 40 | D27 | 1120 | Trifludimoxazin | 12.5 | ERICA | 21 | 85 |
| Bridge 2 | 40 | D27 | 1120 | Trifludimoxazin | 12.5 | ERICA | 21 | 90 |
| Bridge 2 | 41 | D27 | 560 | Trifludimoxazin | 50 | ERICA | 21 | 95 |
| Bridge 2 | 41 | D27 | 560 | Trifludimoxazin | 50 | ERICA | 21 | 99 |
| Bridge 2 | 42 | D27 | 1120 | Trifludimoxazin | 50 | ERICA | 21 | 95 |
| Bridge 2 | 42 | D27 | 1120 | Trifludimoxazin | 50 | ERICA | 21 | 95 |
| Bridge 2 | 43 | D27 | 560 | Saflufenacil | 25 | ERICA | 21 | 80 |
| Bridge 2 | 43 | D27 | 560 | Saflufenacil | 25 | ERICA | 21 | 70 |
| Bridge 2 | 44 | D27 | 1120 | Saflufenacil | 25 | ERICA | 21 | 50 |
| Bridge 2 | 44 | D27 | 1120 | Saflufenacil | 25 | ERICA | 21 | 75 |
| Bridge 2 | 45 | D27 | 560 | Saflufenacil | 75 | ERICA | 21 | 100 |
| Bridge 2 | 45 | D27 | 560 | Saflufenacil | 75 | ERICA | 21 | 100 |
| Bridge 2 | 46 | D27 | 1120 | Saflufenacil | 75 | ERICA | 21 | 75 |
| Bridge 2 | 46 | D27 | 1120 | Saflufenacil | 75 | ERICA | 21 | 100 |
| Bridge 2 | 1 | Solv. Ck | 0 | 0 | 0 | ZEAMX | 21 | 0 |
| | | untreated | | | | | | |
| Bridge 2 | 1 | Solv. Ck | 0 | 0 | 0 | ZEAMX | 21 | 0 |
| | | untreated | | | | | | |
| Bridge 2 | 1 | Solv. Ck | 0 | 0 | 0 | ZEAMX | 21 | 0 |
| | | untreated | | | | | | |
| Bridge 2 | 2 | B2 | 375 | none | | ZEAMX | 21 | 5 |
| Bridge 2 | 2 | B2 | 375 | none | | ZEAMX | 21 | 5 |
| Bridge 2 | 2 | B2 | 375 | none | | ZEAMX | 21 | 5 |
| Bridge 2 | 3 | B2 | 560 | none | | ZEAMX | 21 | 15 |
| Bridge 2 | 3 | B2 | 560 | none | | ZEAMX | 21 | 5 |
| Bridge 2 | 3 | B2 | 560 | none | | ZEAMX | 21 | 5 |
| Bridge 2 | 4 | B2 | 1120 | none | | ZEAMX | 21 | 5 |
| Bridge 2 | 4 | B2 | 1120 | none | | ZEAMX | 21 | 5 |
| Bridge 2 | 4 | B2 | 1120 | none | | ZEAMX | 21 | 5 |
| Bridge 2 | 5 | B2 | 0 | Trifludimoxazin | 12.5 | ZEAMX | 21 | 5 |
| Bridge 2 | 5 | B2 | 0 | Trifludimoxazin | 12.5 | ZEAMX | 21 | 10 |
| Bridge 2 | 5 | B2 | 0 | Trifludimoxazin | 12.5 | ZEAMX | 21 | 5 |
| Bridge 2 | 6 | B2 | 375 | Trifludimoxazin | 12.5 | ZEAMX | 21 | 10 |
| Bridge 2 | 6 | B2 | 375 | Trifludimoxazin | 12.5 | ZEAMX | 21 | 25 |
| Bridge 2 | 6 | B2 | 375 | Trifludimoxazin | 12.5 | ZEAMX | 21 | 20 |
| Bridge 2 | 7 | B2 | 560 | Trifludimoxazin | 12.5 | ZEAMX | 21 | 85 |
| Bridge 2 | 7 | B2 | 560 | Trifludimoxazin | 12.5 | ZEAMX | 21 | 15 |
| Bridge 2 | 7 | B2 | 560 | Trifludimoxazin | 12.5 | ZEAMX | 21 | 30 |
| Bridge 2 | 8 | B2 | 1120 | Trifludimoxazin | 12.5 | ZEAMX | 21 | 25 |
| Bridge 2 | 8 | B2 | 1120 | Trifludimoxazin | 12.5 | ZEAMX | 21 | 30 |
| Bridge 2 | 8 | B2 | 1120 | Trifludimoxazin | 12.5 | ZEAMX | 21 | 15 |
| Bridge 2 | 9 | B2 | 0 | Trifludimoxazin | 25 | ZEAMX | 21 | 15 |
| Bridge 2 | 9 | B2 | 0 | Trifludimoxazin | 25 | ZEAMX | 21 | 5 |
| Bridge 2 | 9 | B2 | 0 | Trifludimoxazin | 25 | ZEAMX | 21 | 10 |
| Bridge 2 | 10 | B2 | 375 | Trifludimoxazin | 25 | ZEAMX | 21 | 30 |
| Bridge 2 | 10 | B2 | 375 | Trifludimoxazin | 25 | ZEAMX | 21 | 15 |
| Bridge 2 | 10 | B2 | 375 | Trifludimoxazin | 25 | ZEAMX | 21 | 15 |
| Bridge 2 | 11 | B2 | 560 | Trifludimoxazin | 25 | ZEAMX | 21 | 20 |
| Bridge 2 | 11 | B2 | 560 | Trifludimoxazin | 25 | ZEAMX | 21 | 20 |
| Bridge 2 | 11 | B2 | 560 | Trifludimoxazin | 25 | ZEAMX | 21 | 10 |
| Bridge 2 | 12 | B2 | 1120 | Trifludimoxazin | 25 | ZEAMX | 21 | 20 |
| Bridge 2 | 12 | B2 | 1120 | Trifludimoxazin | 25 | ZEAMX | 21 | 20 |
| Bridge 2 | 12 | B2 | 1120 | Trifludimoxazin | 25 | ZEAMX | 21 | 30 |
| Bridge 2 | 13 | B2 | 0 | Trifludimoxazin | 50 | ZEAMX | 21 | 15 |
| Bridge 2 | 13 | B2 | 0 | Trifludimoxazin | 50 | ZEAMX | 21 | 20 |
| Bridge 2 | 13 | B2 | 0 | Trifludimoxazin | 50 | ZEAMX | 21 | 10 |
| Bridge 2 | 14 | B2 | 375 | Trifludimoxazin | 50 | ZEAMX | 21 | 30 |
| Bridge 2 | 14 | B2 | 375 | Trifludimoxazin | 50 | ZEAMX | 21 | 20 |
| Bridge 2 | 14 | B2 | 375 | Trifludimoxazin | 50 | ZEAMX | 21 | 15 |
| Bridge 2 | 15 | B2 | 560 | Trifludimoxazin | 50 | ZEAMX | 21 | 25 |
| Bridge 2 | 15 | B2 | 560 | Trifludimoxazin | 50 | ZEAMX | 21 | 35 |
| Bridge 2 | 15 | B2 | 560 | Trifludimoxazin | 50 | ZEAMX | 21 | 55 |
| Bridge 2 | 16 | B2 | 1120 | Trifludimoxazin | 50 | ZEAMX | 21 | 35 |
| Bridge 2 | 16 | B2 | 1120 | Trifludimoxazin | 50 | ZEAMX | 21 | 20 |
| Bridge 2 | 16 | B2 | 1120 | Trifludimoxazin | 50 | ZEAMX | 21 | 15 |
| Bridge 2 | 17 | D10 | 560 | none | 0 | ZEAMX | 21 | 40 |
| Bridge 2 | 17 | D10 | 560 | none | 0 | ZEAMX | 21 | 5 |
| Bridge 2 | 17 | D10 | 560 | none | 0 | ZEAMX | 21 | 10 |
| Bridge 2 | 18 | D10 | 1120 | none | 0 | ZEAMX | 21 | 35 |
| Bridge 2 | 18 | D10 | 1120 | none | 0 | ZEAMX | 21 | 5 |
| Bridge 2 | 18 | D10 | 1120 | none | 0 | ZEAMX | 21 | 10 |
| Bridge 2 | 19 | D10 | 560 | Trifludimoxazin | 12.5 | ZEAMX | 21 | 20 |
| Bridge 2 | 19 | D10 | 560 | Trifludimoxazin | 12.5 | ZEAMX | 21 | 35 |
| Bridge 2 | 19 | D10 | 560 | Trifludimoxazin | 12.5 | ZEAMX | 21 | 5 |
| Bridge 2 | 20 | D10 | 1120 | Trifludimoxazin | 12.5 | ZEAMX | 21 | 15 |
| Bridge 2 | 20 | D10 | 1120 | Trifludimoxazin | 12.5 | ZEAMX | 21 | 15 |
| Bridge 2 | 20 | D10 | 1120 | Trifludimoxazin | 12.5 | ZEAMX | 21 | 10 |
| Bridge 2 | 21 | D10 | 560 | Trifludimoxazin | 50 | ZEAMX | 21 | 15 |
| Bridge 2 | 21 | D10 | 560 | Trifludimoxazin | 50 | ZEAMX | 21 | 10 |
| Bridge 2 | 21 | D10 | 560 | Trifludimoxazin | 50 | ZEAMX | 21 | 20 |
| Bridge 2 | 22 | D10 | 1120 | Trifludimoxazin | 50 | ZEAMX | 21 | 30 |
| Bridge 2 | 22 | D10 | 1120 | Trifludimoxazin | 50 | ZEAMX | 21 | 15 |
| Bridge 2 | 22 | D10 | 1120 | Trifludimoxazin | 50 | ZEAMX | 21 | 25 |
| Bridge 2 | 23 | D10 | 560 | Saflufenacil | 25 | ZEAMX | 21 | 35 |
| Bridge 2 | 23 | D10 | 560 | Saflufenacil | 25 | ZEAMX | 21 | 10 |
| Bridge 2 | 23 | D10 | 560 | Saflufenacil | 25 | ZEAMX | 21 | 20 |
| Bridge 2 | 24 | D10 | 1120 | Saflufenacil | 25 | ZEAMX | 21 | 25 |
| Bridge 2 | 24 | D10 | 1120 | Saflufenacil | 25 | ZEAMX | 21 | 15 |
| Bridge 2 | 24 | D10 | 1120 | Saflufenacil | 25 | ZEAMX | 21 | 10 |
| Bridge 2 | 25 | D10 | 560 | Saflufenacil | 75 | ZEAMX | 21 | 20 |
| Bridge 2 | 25 | D10 | 560 | Saflufenacil | 75 | ZEAMX | 21 | 40 |
| Bridge 2 | 25 | D10 | 560 | Saflufenacil | 75 | ZEAMX | 21 | 15 |
| Bridge 2 | 26 | D10 | 1120 | Saflufenacil | 75 | ZEAMX | 21 | 50 |
| Bridge 2 | 26 | D10 | 1120 | Saflufenacil | 75 | ZEAMX | 21 | 40 |
| Bridge 2 | 26 | D10 | 1120 | Saflufenacil | 75 | ZEAMX | 21 | 35 |
| Bridge 2 | 27 | D8 | 560 | none | 0 | ZEAMX | 21 | 5 |
| Bridge 2 | 27 | D8 | 560 | none | 0 | ZEAMX | 21 | 10 |
| Bridge 2 | 27 | D8 | 560 | none | 0 | ZEAMX | 21 | 5 |
| Bridge 2 | 28 | D8 | 1120 | none | 0 | ZEAMX | 21 | 25 |
| Bridge 2 | 28 | D8 | 1120 | none | 0 | ZEAMX | 21 | 10 |
| Bridge 2 | 28 | D8 | 1120 | none | 0 | ZEAMX | 21 | 5 |
| Bridge 2 | 29 | D8 | 560 | Trifludimoxazin | 12.5 | ZEAMX | 21 | 20 |
| Bridge 2 | 29 | D8 | 560 | Trifludimoxazin | 12.5 | ZEAMX | 21 | 20 |
| Bridge 2 | 29 | D8 | 560 | Trifludimoxazin | 12.5 | ZEAMX | 21 | 15 |
| Bridge 2 | 30 | D8 | 1120 | Trifludimoxazin | 12.5 | ZEAMX | 21 | 20 |
| Bridge 2 | 30 | D8 | 1120 | Trifludimoxazin | 12.5 | ZEAMX | 21 | 15 |
| Bridge 2 | 30 | D8 | 1120 | Trifludimoxazin | 12.5 | ZEAMX | 21 | 20 |
| Bridge 2 | 31 | D8 | 560 | Trifludimoxazin | 50 | ZEAMX | 21 | 50 |
| Bridge 2 | 31 | D8 | 560 | Trifludimoxazin | 50 | ZEAMX | 21 | 25 |
| Bridge 2 | 31 | D8 | 560 | Trifludimoxazin | 50 | ZEAMX | 21 | 30 |
| Bridge 2 | 32 | D8 | 1120 | Trifludimoxazin | 50 | ZEAMX | 21 | 35 |
| Bridge 2 | 32 | D8 | 1120 | Trifludimoxazin | 50 | ZEAMX | 21 | 40 |
| Bridge 2 | 32 | D8 | 1120 | Trifludimoxazin | 50 | ZEAMX | 21 | 20 |
| Bridge 2 | 33 | D8 | 560 | Saflufenacil | 25 | ZEAMX | 21 | 70 |
| Bridge 2 | 33 | D8 | 560 | Saflufenacil | 25 | ZEAMX | 21 | 40 |
| Bridge 2 | 33 | D8 | 560 | Saflufenacil | 25 | ZEAMX | 21 | 55 |
| Bridge 2 | 34 | D8 | 1120 | Saflufenacil | 25 | ZEAMX | 21 | 80 |
| Bridge 2 | 34 | D8 | 1120 | Saflufenacil | 25 | ZEAMX | 21 | 70 |
| Bridge 2 | 34 | D8 | 1120 | Saflufenacil | 25 | ZEAMX | 21 | 65 |
| Bridge 2 | 35 | D8 | 560 | Saflufenacil | 75 | ZEAMX | 21 | 65 |
| Bridge 2 | 35 | D8 | 560 | Saflufenacil | 75 | ZEAMX | 21 | 60 |
| Bridge 2 | 35 | D8 | 560 | Saflufenacil | 75 | ZEAMX | 21 | 50 |
| Bridge 2 | 36 | D8 | 1120 | Saflufenacil | 75 | ZEAMX | 21 | 70 |
| Bridge 2 | 36 | D8 | 1120 | Saflufenacil | 75 | ZEAMX | 21 | 65 |
| Bridge 2 | 36 | D8 | 1120 | Saflufenacil | 75 | ZEAMX | 21 | 85 |
| Bridge 2 | 37 | D27 | 560 | none | 0 | ZEAMX | 21 | 15 |
| Bridge 2 | 37 | D27 | 560 | none | 0 | ZEAMX | 21 | 5 |
| Bridge 2 | 37 | D27 | 560 | none | 0 | ZEAMX | 21 | 5 |
| Bridge 2 | 38 | D27 | 1120 | none | 0 | ZEAMX | 21 | 30 |
| Bridge 2 | 38 | D27 | 1120 | none | 0 | ZEAMX | 21 | 15 |
| Bridge 2 | 38 | D27 | 1120 | none | 0 | ZEAMX | 21 | 5 |
| Bridge 2 | 39 | D27 | 560 | Trifludimoxazin | 12.5 | ZEAMX | 21 | 40 |
| Bridge 2 | 39 | D27 | 560 | Trifludimoxazin | 12.5 | ZEAMX | 21 | 20 |
| Bridge 2 | 39 | D27 | 560 | Trifludimoxazin | 12.5 | ZEAMX | 21 | 25 |
| Bridge 2 | 40 | D27 | 1120 | Trifludimoxazin | 12.5 | ZEAMX | 21 | 20 |
| Bridge 2 | 40 | D27 | 1120 | Trifludimoxazin | 12.5 | ZEAMX | 21 | 40 |
| Bridge 2 | 40 | D27 | 1120 | Trifludimoxazin | 12.5 | ZEAMX | 21 | 30 |
| Bridge 2 | 41 | D27 | 560 | Trifludimoxazin | 50 | ZEAMX | 21 | 35 |
| Bridge 2 | 41 | D27 | 560 | Trifludimoxazin | 50 | ZEAMX | 21 | 10 |
| Bridge 2 | 41 | D27 | 560 | Trifludimoxazin | 50 | ZEAMX | 21 | 5 |
| Bridge 2 | 42 | D27 | 1120 | Trifludimoxazin | 50 | ZEAMX | 21 | 25 |
| Bridge 2 | 42 | D27 | 1120 | Trifludimoxazin | 50 | ZEAMX | 21 | 5 |
| Bridge 2 | 42 | D27 | 1120 | Trifludimoxazin | 50 | ZEAMX | 21 | 10 |
| Bridge 2 | 43 | D27 | 560 | Saflufenacil | 25 | ZEAMX | 21 | 40 |
| Bridge 2 | 43 | D27 | 560 | Saflufenacil | 25 | ZEAMX | 21 | 60 |
| Bridge 2 | 43 | D27 | 560 | Saflufenacil | 25 | ZEAMX | 21 | 50 |
| Bridge 2 | 44 | D27 | 1120 | Saflufenacil | 25 | ZEAMX | 21 | 60 |
| Bridge 2 | 44 | D27 | 1120 | Saflufenacil | 25 | ZEAMX | 21 | 25 |
| Bridge 2 | 44 | D27 | 1120 | Saflufenacil | 25 | ZEAMX | 21 | 55 |
| Bridge 2 | 45 | D27 | 560 | Saflufenacil | 75 | ZEAMX | 21 | 75 |
| Bridge 2 | 45 | D27 | 560 | Saflufenacil | 75 | ZEAMX | 21 | 50 |
| Bridge 2 | 45 | D27 | 560 | Saflufenacil | 75 | ZEAMX | 21 | 80 |
| Bridge 2 | 46 | D27 | 1120 | Saflufenacil | 75 | ZEAMX | 21 | 90 |
| Bridge 2 | 46 | D27 | 1120 | Saflufenacil | 75 | ZEAMX | 21 | 60 |
| Bridge 2 | 46 | D27 | 1120 | Saflufenacil | 75 | ZEAMX | 21 | 75 |
| Bridge 3 | 1 | Solv. Ck | 0 | 0 | 0 | ABUTH | 21 | 0 |
| | | untreated | | | | | | |
| Bridge 3 | 1 | Solv. Ck | 0 | 0 | 0 | ABUTH | 21 | 0 |
| | | untreated | | | | | | |
| Bridge 3 | 1 | Solv. Ck | 0 | 0 | 0 | ABUTH | 21 | 0 |
| | | untreated | | | | | | |
| Bridge 3 | 2 | B2 | 375 | none | | ABUTH | 21 | 80 |
| Bridge 3 | 2 | B2 | 375 | none | | ABUTH | 21 | 95 |
| Bridge 3 | 2 | B2 | 375 | none | | ABUTH | 21 | 90 |
| Bridge 3 | 3 | B2 | 560 | none | | ABUTH | 21 | 85 |
| Bridge 3 | 3 | B2 | 560 | none | | ABUTH | 21 | 85 |
| Bridge 3 | 3 | B2 | 560 | none | | ABUTH | 21 | 90 |
| Bridge 3 | 4 | B2 | 1120 | none | | ABUTH | 21 | 85 |
| Bridge 3 | 4 | B2 | 1120 | none | | ABUTH | 21 | 97 |
| Bridge 3 | 4 | B2 | 1120 | none | | ABUTH | 21 | 97 |
| Bridge 3 | 5 | B2 | 0 | Sulfentrazone | 140 | ABUTH | 21 | 98 |
| Bridge 3 | 5 | B2 | 0 | Sulfentrazone | 140 | ABUTH | 21 | 100 |
| Bridge 3 | 5 | B2 | 0 | Sulfentrazone | 140 | ABUTH | 21 | 100 |
| Bridge 3 | 6 | B2 | 375 | Sulfentrazone | 140 | ABUTH | 21 | 75 |
| Bridge 3 | 6 | B2 | 375 | Sulfentrazone | 140 | ABUTH | 21 | 99 |
| Bridge 3 | 6 | B2 | 375 | Sulfentrazone | 140 | ABUTH | 21 | 100 |
| Bridge 3 | 7 | B2 | 560 | Sulfentrazone | 140 | ABUTH | 21 | 100 |
| Bridge 3 | 7 | B2 | 560 | Sulfentrazone | 140 | ABUTH | 21 | 99 |
| Bridge 3 | 7 | B2 | 560 | Sulfentrazone | 140 | ABUTH | 21 | 92 |
| Bridge 3 | 8 | B2 | 1120 | Sulfentrazone | 140 | ABUTH | 21 | 100 |
| Bridge 3 | 8 | B2 | 1120 | Sulfentrazone | 140 | ABUTH | 21 | 100 |
| Bridge 3 | 8 | B2 | 1120 | Sulfentrazone | 140 | ABUTH | 21 | 100 |
| Bridge 3 | 9 | B2 | 0 | Sulfentrazone | 216 | ABUTH | 21 | 08 |
| Bridge 3 | 9 | B2 | 0 | Sulfentrazone | 210 | ABUTH | 21 | 100 |
| Bridge 3 | 9 | B2 | 0 | Sulfentrazone | 210 | ABUTH | 21 | 100 |
| Bridge 3 | 10 | B2 | 375 | Sulfentrazone | 210 | ABUTH | 21 | 100 |
| Bridge 3 | 10 | B2 | 375 | Sulfentrazone | 210 | ABUTH | 21 | 100 |
| Bridge 3 | 10 | B2 | 375 | Sulfentrazone | 210 | ABUTH | 21 | 100 |
| Bridge 3 | 11 | B2 | 560 | Sulfentrazone | 210 | ABUTH | 21 | 100 |
| Bridge 3 | 11 | B2 | 560 | Sulfentrazone | 210 | ABUTH | 21 | 100 |
| Bridge 3 | 11 | B2 | 560 | Sulfentrazone | 210 | ABUTH | 21 | 100 |
| Bridge 3 | 12 | B2 | 1120 | Sulfentrazone | 210 | ABUTH | 21 | 100 |
| Bridge 3 | 12 | B2 | 1120 | Sulfentrazone | 210 | ABUTH | 21 | 100 |
| Bridge 3 | 12 | B2 | 1120 | Sulfentrazone | 210 | ABUTH | 21 | 100 |
| Bridge 3 | 13 | B2 | 0 | Sulfentrazone | 420 | ABUTH | 21 | 70 |
| Bridge 3 | 13 | B2 | 0 | Sulfentrazone | 420 | ABUTH | 21 | 100 |
| Bridge 3 | 13 | B2 | 0 | Sulfentrazone | 420 | ABUTH | 21 | 100 |
| Bridge 3 | 14 | B2 | 375 | Sulfentrazone | 420 | ABUTH | 21 | 100 |
| Bridge 3 | 14 | B2 | 375 | Sulfentrazone | 420 | ABUTH | 21 | 100 |
| Bridge 3 | 14 | B2 | 375 | Sulfentrazone | 420 | ABUTH | 21 | 100 |
| Bridge 3 | 15 | B2 | 560 | Sulfentrazone | 420 | ABUTH | 21 | 100 |
| Bridge 3 | 15 | B2 | 560 | Sulfentrazone | 420 | ABUTH | 21 | 100 |
| Bridge 3 | 15 | B2 | 560 | Sulfentrazone | 420 | ABUTH | 21 | 100 |
| Bridge 3 | 16 | B2 | 1120 | Sulfentrazone | 420 | ABUTH | 21 | 100 |
| Bridge 3 | 16 | B2 | 1120 | Sulfentrazone | 420 | ABUTH | 21 | 100 |
| Bridge 3 | 16 | B2 | 1120 | Sulfentrazone | 420 | ABUTH | 21 | 100 |
| Bridge 3 | 17 | D10 | 560 | none | 0 | ABUTH | 21 | 85 |
| Bridge 3 | 17 | D10 | 560 | none | 0 | ABUTH | 21 | 90 |
| Bridge 3 | 17 | D10 | 560 | none | 0 | ABUTH | 21 | 97 |
| Bridge 3 | 18 | D10 | 1120 | none | 0 | ABUTH | 21 | 95 |
| Bridge 3 | 18 | D10 | 1120 | none | 0 | ABUTH | 21 | 97 |
| Bridge 3 | 18 | D10 | 1120 | none | 0 | ABUTH | 21 | 99 |
| Bridge 3 | 19 | D10 | 560 | Sulfentrazone | 130 | ABUTH | 21 | 99 |
| Bridge 3 | 19 | D10 | 560 | Sulfentrazone | 130 | ABUTH | 21 | 100 |
| Bridge 3 | 19 | D10 | 560 | Sulfentrazone | 130 | ABUTH | 21 | 100 |
| Bridge 3 | 20 | D10 | 1120 | Sulfentrazone | 130 | ABUTH | 21 | 100 |
| Bridge 3 | 20 | D10 | 1120 | Sulfentrazone | 130 | ABUTH | 21 | 100 |
| Bridge 3 | 20 | D10 | 1120 | Sulfentrazone | 130 | ABUTH | 21 | 100 |
| Bridge 3 | 21 | D10 | 560 | Sulfentrazone | 347 | ABUTH | 21 | 90 |
| Bridge 3 | 21 | D10 | 560 | Sulfentrazone | 347 | ABUTH | 21 | 99 |
| Bridge 3 | 21 | D10 | 560 | Sulfentrazone | 347 | ABUTH | 21 | 90 |
| Bridge 3 | 22 | D10 | 1120 | Sulfentrazone | 347 | ABUTH | 21 | 100 |
| Bridge 3 | 22 | D10 | 1120 | Sulfentrazone | 347 | ABUTH | 21 | 100 |
| Bridge 3 | 22 | D10 | 1120 | Sulfentrazone | 347 | ABUTH | 21 | 100 |
| Bridge 3 | 23 | D8 | 560 | none | 0 | ABUTH | 21 | 90 |
| Bridge 3 | 23 | D8 | 560 | none | 0 | ABUTH | 21 | 95 |
| Bridge 3 | 23 | D8 | 560 | none | 0 | ABUTH | 21 | 100 |
| Bridge 3 | 24 | D8 | 1120 | none | 0 | ABUTH | 21 | 95 |
| Bridge 3 | 24 | D8 | 1120 | none | 0 | ABUTH | 21 | 95 |
| Bridge 3 | 24 | D8 | 1120 | none | 0 | ABUTH | 21 | 97 |
| Bridge 3 | 25 | D8 | 560 | Sulfentrazone | 130 | ABUTH | 21 | 100 |
| Bridge 3 | 25 | D8 | 560 | Sulfentrazone | 130 | ABUTH | 21 | 100 |
| Bridge 3 | 25 | D8 | 560 | Sulfentrazone | 130 | ABUTH | 21 | 100 |
| Bridge 3 | 26 | D8 | 1120 | Sulfentrazone | 130 | ABUTH | 21 | 100 |
| Bridge 3 | 26 | D8 | 1120 | Sulfentrazone | 130 | ABUTH | 21 | 100 |
| Bridge 3 | 26 | D8 | 1120 | Sulfentrazone | 130 | ABUTH | 21 | 100 |
| Bridge 3 | 27 | D8 | 560 | Sulfentrazone | 347 | ABUTH | 21 | 99 |
| Bridge 3 | 27 | D8 | 560 | Sulfentrazone | 347 | ABUTH | 21 | 100 |
| Bridge 3 | 27 | D8 | 560 | Sulfentrazone | 347 | ABUTH | 21 | 100 |
| Bridge 3 | 28 | D8 | 1120 | Sulfentrazone | 347 | ABUTH | 21 | 100 |
| Bridge 3 | 28 | D8 | 1120 | Sulfentrazone | 347 | ABUTH | 21 | 100 |
| Bridge 3 | 28 | D8 | 1120 | Sulfentrazone | 347 | ABUTH | 21 | 100 |
| Bridge 3 | 29 | D27 | 560 | none | 0 | ABUTH | 21 | 100 |
| Bridge 3 | 29 | D27 | 560 | none | 0 | ABUTH | 21 | 99 |
| Bridge 3 | 29 | D27 | 560 | none | 0 | ABUTH | 21 | 95 |
| Bridge 3 | 30 | D27 | 1120 | none | 0 | ABUTH | 21 | 99 |
| Bridge 3 | 30 | D27 | 1120 | none | 0 | ABUTH | 21 | 97 |
| Bridge 3 | 30 | D27 | 1120 | none | 0 | ABUTH | 21 | 95 |
| Bridge 3 | 31 | D27 | 560 | Sulfentrazone | 130 | ABUTH | 21 | 99 |
| Bridge 3 | 31 | D27 | 560 | Sulfentrazone | 130 | ABUTH | 21 | 100 |
| Bridge 3 | 31 | D27 | 560 | Sulfentrazone | 130 | ABUTH | 21 | 100 |
| Bridge 3 | 32 | D27 | 1120 | Sulfentrazone | 130 | ABUTH | 21 | 100 |
| Bridge 3 | 32 | D27 | 1120 | Sulfentrazone | 130 | ABUTH | 21 | 100 |
| Bridge 3 | 32 | D27 | 1120 | Sulfentrazone | 130 | ABUTH | 21 | 100 |
| Bridge 3 | 33 | D27 | 560 | Sulfentrazone | 347 | ABUTH | 21 | 100 |
| Bridge 3 | 33 | D27 | 560 | Sulfentrazone | 347 | ABUTH | 21 | 100 |
| Bridge 3 | 33 | D27 | 560 | Sulfentrazone | 347 | ABUTH | 21 | 100 |
| Bridge 3 | 34 | D27 | 1120 | Sulfentrazone | 347 | ABUTH | 21 | 100 |
| Bridge 3 | 34 | D27 | 1120 | Sulfentrazone | 347 | ABUTH | 21 | 100 |
| Bridge 3 | 34 | D27 | 1120 | Sulfentrazone | 347 | ABUTH | 21 | 100 |
| Bridge 3 | 35 | Untreated | | | | ABUTH | 21 | 0 |
| Bridge 3 | 35 | Untreated | | | | ABUTH | 21 | 0 |
| Bridge 3 | 35 | Untreated | | | | ABUTH | 21 | 0 |
| Bridge 3 | 1 | Solv. Ck | 0 | 0 | 0 | AMATA | 21 | 0 |
| | | untreated | | | | | | |
| Bridge 3 | 1 | Solv. Ck | 0 | 0 | 0 | AMATA | 21 | 0 |
| | | untreated | | | | | | |
| Bridge 3 | 1 | Solv. Ck | 0 | 0 | 0 | AMATA | 21 | 0 |
| | | untreated | | | | | | |
| Bridge 3 | 2 | B2 | 375 | none | | AMATA | 21 | 100 |
| Bridge 3 | 2 | B2 | 375 | none | | AMATA | 21 | 100 |
| Bridge 3 | 2 | B2 | 375 | none | | AMATA | 21 | 100 |
| Bridge 3 | 3 | B2 | 560 | none | | AMATA | 21 | 100 |
| Bridge 3 | 3 | B2 | 560 | none | | AMATA | 21 | 100 |
| Bridge 3 | 3 | B2 | 560 | none | | AMATA | 21 | 100 |
| Bridge 3 | 4 | B2 | 1120 | none | | AMATA | 21 | 100 |
| Bridge 3 | 4 | B2 | 1120 | none | | AMATA | 21 | 100 |
| Bridge 3 | 4 | B2 | 1120 | none | | AMATA | 21 | 100 |
| Bridge 3 | 5 | B2 | 0 | Sulfentrazone | 140 | AMATA | 21 | 100 |
| Bridge 3 | 5 | B2 | 0 | Sulfentrazone | 140 | AMATA | 21 | 100 |
| Bridge 3 | 5 | B2 | 0 | Sulfentrazone | 140 | AMATA | 21 | 100 |
| Bridge 3 | 6 | B2 | 375 | Sulfentrazone | 140 | AMATA | 21 | 100 |
| Bridge 3 | 6 | B2 | 375 | Sulfentrazone | 140 | AMATA | 21 | 100 |
| Bridge 3 | 6 | B2 | 375 | Sulfentrazone | 140 | AMATA | 21 | 100 |
| Bridge 3 | 7 | B2 | 560 | Sulfentrazone | 140 | AMATA | 21 | 100 |
| Bridge 3 | 7 | B2 | 560 | Sulfentrazone | 140 | AMATA | 21 | 100 |
| Bridge 3 | 7 | B2 | 560 | Sulfentrazone | 140 | AMATA | 21 | 100 |
| Bridge 3 | 8 | B2 | 1120 | Sulfentrazone | 140 | AMATA | 21 | 100 |
| Bridge 3 | 8 | B2 | 1120 | Sulfentrazone | 140 | AMATA | 21 | 100 |
| Bridge 3 | 8 | B2 | 1120 | Sulfentrazone | 140 | AMATA | 21 | 100 |
| Bridge 3 | 9 | B2 | 0 | Sulfentrazone | 210 | AMATA | 21 | 100 |
| Bridge 3 | 9 | B2 | 0 | Sulfentrazone | 210 | AMATA | 21 | 100 |
| Bridge 3 | 9 | B2 | 0 | Sulfentrazone | 210 | AMATA | 21 | 100 |
| Bridge 3 | 10 | B2 | 375 | Sulfentrazone | 210 | AMATA | 21 | 100 |
| Bridge 3 | 10 | B2 | 375 | Sulfentrazone | 210 | AMATA | 21 | 100 |
| Bridge 3 | 10 | B2 | 375 | Sulfentrazone | 210 | AMATA | 21 | 100 |
| Bridge 3 | 11 | B2 | 560 | Sulfentrazone | 210 | AMATA | 21 | 100 |
| Bridge 3 | 11 | B2 | 560 | Sulfentrazone | 210 | AMATA | 21 | 100 |
| Bridge 3 | 11 | B2 | 560 | Sulfentrazone | 210 | AMATA | 21 | 100 |
| Bridge 3 | 12 | B2 | 1120 | Sulfentrazone | 210 | AMATA | 21 | 100 |
| Bridge 3 | 12 | B2 | 1120 | Sulfentrazone | 210 | AMATA | 21 | 100 |
| Bridge 3 | 12 | B2 | 1120 | Sulfentrazone | 210 | AMATA | 21 | 100 |
| Bridge 3 | 13 | B2 | 0 | Sulfentrazone | 420 | AMATA | 21 | 100 |
| Bridge 3 | 13 | B2 | 0 | Sulfentrazone | 420 | AMATA | 21 | 100 |
| Bridge 3 | 13 | B2 | 0 | Sulfentrazone | 420 | AMATA | 21 | 100 |
| Bridge 3 | 14 | B2 | 375 | Sulfentrazone | 420 | AMATA | 21 | 100 |
| Bridge 3 | 14 | B2 | 375 | Sulfentrazone | 420 | AMATA | 21 | 100 |
| Bridge 3 | 14 | B2 | 375 | Sulfentrazone | 420 | AMATA | 21 | 100 |
| Bridge 3 | 15 | B2 | 560 | Sulfentrazone | 420 | AMATA | 21 | 100 |
| Bridge 3 | 15 | B2 | 560 | Sulfentrazone | 420 | AMATA | 21 | 100 |
| Bridge 3 | 15 | B2 | 560 | Sulfentrazone | 420 | AMATA | 21 | 100 |
| Bridge 3 | 16 | B2 | 1120 | Sulfentrazone | 420 | AMATA | 21 | 100 |
| Bridge 3 | 16 | B2 | 1120 | Sulfentrazone | 420 | AMATA | 21 | 100 |
| Bridge 3 | 16 | B2 | 1120 | Sulfentrazone | 420 | AMATA | 21 | 100 |
| Bridge 3 | 17 | D10 | 560 | none | 0 | AMATA | 21 | 100 |
| Bridge 3 | 17 | D10 | 560 | none | 0 | AMATA | 21 | 99 |
| Bridge 3 | 17 | D10 | 560 | none | 0 | AMATA | 21 | 100 |
| Bridge 3 | 18 | D10 | 1120 | none | 0 | AMATA | 21 | 100 |
| Bridge 3 | 18 | D10 | 1120 | none | 0 | AMATA | 21 | 100 |
| Bridge 3 | 18 | D10 | 1120 | none | 0 | AMATA | 21 | 100 |
| Bridge 3 | 19 | D10 | 560 | Sulfentrazone | 130 | AMATA | 21 | 100 |
| Bridge 3 | 19 | D10 | 560 | Sulfentrazone | 130 | AMATA | 21 | 100 |
| Bridge 3 | 19 | D10 | 560 | Sulfentrazone | 130 | AMATA | 21 | 100 |
| Bridge 3 | 20 | D10 | 1120 | Sulfentrazone | 130 | AMATA | 21 | 100 |
| Bridge 3 | 20 | D10 | 1120 | Sulfentrazone | 130 | AMATA | 21 | 100 |
| Bridge 3 | 20 | D10 | 1120 | Sulfentrazone | 130 | AMATA | 21 | 100 |
| Bridge 3 | 21 | D10 | 560 | Sulfentrazone | 347 | AMATA | 21 | 100 |
| Bridge 3 | 21 | D10 | 560 | Sulfentrazone | 347 | AMATA | 21 | 100 |
| Bridge 3 | 21 | D10 | 560 | Sulfentrazone | 347 | AMATA | 21 | 100 |
| Bridge 3 | 22 | D10 | 1120 | Sulfentrazone | 347 | AMATA | 21 | 100 |
| Bridge 3 | 22 | D10 | 1120 | Sulfentrazone | 347 | AMATA | 21 | 100 |
| Bridge 3 | 22 | D10 | 1120 | Sulfentrazone | 347 | AMATA | 21 | 100 |
| Bridge 3 | 23 | D8 | 560 | none | 0 | AMATA | 21 | 100 |
| Bridge 3 | 23 | D8 | 560 | none | 0 | AMATA | 21 | 100 |
| Bridge 3 | 23 | D8 | 560 | none | 0 | AMATA | 21 | 90 |
| Bridge 3 | 24 | D8 | 1120 | none | 0 | AMATA | 21 | 100 |
| Bridge 3 | 24 | D8 | 1120 | none | 0 | AMATA | 21 | 100 |
| Bridge 3 | 24 | D8 | 1120 | none | 0 | AMATA | 21 | 100 |
| Bridge 3 | 25 | D8 | 560 | Sulfentrazone | 130 | AMATA | 21 | 100 |
| Bridge 3 | 25 | D8 | 560 | Sulfentrazone | 130 | AMATA | 21 | 100 |
| Bridge 3 | 25 | D8 | 560 | Sulfentrazone | 130 | AMATA | 21 | 100 |
| Bridge 3 | 26 | D8 | 1120 | Sulfentrazone | 130 | AMATA | 21 | 100 |
| Bridge 3 | 26 | D8 | 1120 | Sulfentrazone | 130 | AMATA | 21 | 100 |
| Bridge 3 | 26 | D8 | 1120 | Sulfentrazone | 130 | AMATA | 21 | 100 |
| Bridge 3 | 27 | D8 | 560 | Sulfentrazone | 347 | AMATA | 21 | 100 |
| Bridge 3 | 27 | D8 | 560 | Sulfentrazone | 347 | AMATA | 21 | 100 |
| Bridge 3 | 27 | D8 | 560 | Sulfentrazone | 347 | AMATA | 21 | 100 |
| Bridge 3 | 28 | D8 | 1120 | Sulfentrazone | 347 | AMATA | 21 | 100 |
| Bridge 3 | 28 | D8 | 1120 | Sulfentrazone | 347 | AMATA | 21 | 100 |
| Bridge 3 | 28 | D8 | 1120 | Sulfentrazone | 347 | AMATA | 21 | 100 |
| Bridge 3 | 29 | D27 | 560 | none | 0 | AMATA | 21 | 100 |
| Bridge 3 | 29 | D27 | 560 | none | 0 | AMATA | 21 | 95 |
| Bridge 3 | 29 | D27 | 560 | none | 0 | AMATA | 21 | 100 |
| Bridge 3 | 30 | D27 | 1120 | none | 0 | AMATA | 21 | 100 |
| Bridge 3 | 30 | D27 | 1120 | none | 0 | AMATA | 21 | 100 |
| Bridge 3 | 30 | D27 | 1120 | none | 0 | AMATA | 21 | 100 |
| Bridge 3 | 31 | D27 | 560 | Sulfentrazone | 130 | AMATA | 21 | 100 |
| Bridge 3 | 31 | D27 | 560 | Sulfentrazone | 130 | AMATA | 21 | 100 |
| Bridge 3 | 31 | D27 | 560 | Sulfentrazone | 130 | AMATA | 21 | 100 |
| Bridge 3 | 32 | D27 | 1120 | Sulfentrazone | 130 | AMATA | 21 | 100 |
| Bridge 3 | 32 | D27 | 1120 | Sulfentrazone | 130 | AMATA | 21 | 100 |
| Bridge 3 | 32 | D27 | 1120 | Sulfentrazone | 130 | AMATA | 21 | 100 |
| Bridge 3 | 33 | D27 | 560 | Sulfentrazone | 347 | AMATA | 21 | 100 |
| Bridge 3 | 33 | D27 | 560 | Sulfentrazone | 347 | AMATA | 21 | 100 |
| Bridge 3 | 33 | D27 | 560 | Sulfentrazone | 347 | AMATA | 21 | 100 |
| Bridge 3 | 34 | D27 | 1120 | Sulfentrazone | 347 | AMATA | 21 | 100 |
| Bridge 3 | 34 | D27 | 1120 | Sulfentrazone | 347 | AMATA | 21 | 100 |
| Bridge 3 | 34 | D27 | 1120 | Sulfentrazone | 347 | AMATA | 21 | 100 |
| Bridge 3 | 35 | Untreated | | | | AMATA | 21 | 0 |
| Bridge 3 | 35 | Untreated | | | | AMATA | 21 | 0 |
| Bridge 3 | 35 | Untreated | | | | AMATA | 21 | 0 |
| Bridge 3 | 1 | Solv. Ck | 0 | 0 | 0 | CHEAL | 21 | 0 |
| | | untreated | | | | | | |
| Bridge 3 | 1 | Solv. Ck | 0 | 0 | 0 | CHEAL | 21 | 0 |
| | | untreated | | | | | | |
| Bridge 3 | 1 | Solv. Ck | 0 | 0 | 0 | CHEAL | 21 | 0 |
| | | untreated | | | | | | |
| Bridge 3 | 2 | B2 | 375 | none | | CHEAL | 21 | 95 |
| Bridge 3 | 2 | B2 | 375 | none | | CHEAL | 21 | 95 |
| Bridge 3 | 2 | B2 | 375 | none | | CHEAL | 21 | 100 |
| Bridge 3 | 3 | B2 | 560 | none | | CHEAL | 21 | 95 |
| Bridge 3 | 3 | B2 | 560 | none | | CHEAL | 21 | 95 |
| Bridge 3 | 3 | B2 | 560 | none | | CHEAL | 21 | 95 |
| Bridge 3 | 4 | B2 | 1120 | none | | CHEAL | 21 | 98 |
| Bridge 3 | 4 | B2 | 1120 | none | | CHEAL | 21 | 100 |
| Bridge 3 | 4 | B2 | 1120 | none | | CHEAL | 21 | 100 |
| Bridge 3 | 5 | B2 | 0 | Sulfentrazone | 140 | CHEAL | 21 | 99 |
| Bridge 3 | 5 | B2 | 0 | Sulfentrazone | 140 | CHEAL | 21 | 100 |
| Bridge 3 | 5 | B2 | 0 | Sulfentrazone | 140 | CHEAL | 21 | 100 |
| Bridge 3 | 6 | B2 | 375 | Sulfentrazone | 140 | CHEAL | 21 | 100 |
| Bridge 3 | 6 | B2 | 375 | Sulfentrazone | 140 | CHEAL | 21 | 100 |
| Bridge 3 | 6 | B2 | 375 | Sulfentrazone | 140 | CHEAL | 21 | 100 |
| Bridge 3 | 7 | B2 | 560 | Sulfentrazone | 140 | CHEAL | 21 | 100 |
| Bridge 3 | 7 | B2 | 560 | Sulfentrazone | 140 | CHEAL | 21 | 100 |
| Bridge 3 | 7 | B2 | 560 | Sulfentrazone | 140 | CHEAL | 21 | 100 |
| Bridge 3 | 8 | B2 | 1120 | Sulfentrazone | 140 | CHEAL | 21 | 100 |
| Bridge 3 | 8 | B2 | 1120 | Sulfentrazone | 140 | CHEAL | 21 | 100 |
| Bridge 3 | 8 | B2 | 1120 | Sulfentrazone | 140 | CHEAL | 21 | 100 |
| Bridge 3 | 9 | B2 | 0 | Sulfentrazone | 210 | CHEAL | 21 | 88 |
| Bridge 3 | 9 | B2 | 0 | Sulfentrazone | 210 | CHEAL | 21 | 100 |
| Bridge 3 | 9 | B2 | 0 | Sulfentrazone | 210 | CHEAL | 21 | 100 |
| Bridge 3 | 10 | B2 | 375 | Sulfentrazone | 210 | CHEAL | 21 | 100 |
| Bridge 3 | 10 | B2 | 375 | Sulfentrazone | 210 | CHEAL | 21 | 100 |
| Bridge 3 | 10 | B2 | 375 | Sulfentrazone | 210 | CHEAL | 21 | 100 |
| Bridge 3 | 11 | B2 | 560 | Sulfentrazone | 210 | CHEAL | 21 | 100 |
| Bridge 3 | 11 | B2 | 560 | Sulfentrazone | 210 | CHEAL | 21 | 100 |
| Bridge 3 | 11 | B2 | 560 | Sulfentrazone | 210 | CHEAL | 21 | 100 |
| Bridge 3 | 12 | B2 | 1120 | Sulfentrazone | 210 | CHEAL | 21 | 100 |
| Bridge 3 | 12 | B2 | 1120 | Sulfentrazone | 210 | CHEAL | 21 | 100 |
| Bridge 3 | 12 | B2 | 1120 | Sulfentrazone | 210 | CHEAL | 21 | 100 |
| Bridge 3 | 13 | B2 | 0 | Sulfentrazone | 420 | CHEAL | 21 | 100 |
| Bridge 3 | 13 | B2 | 0 | Sulfentrazone | 420 | CHEAL | 21 | 100 |
| Bridge 3 | 13 | B2 | 0 | Sulfentrazone | 420 | CHEAL | 21 | 100 |
| Bridge 3 | 14 | B2 | 375 | Sulfentrazone | 420 | CHEAL | 21 | 100 |
| Bridge 3 | 14 | B2 | 375 | Sulfentrazone | 420 | CHEAL | 21 | 100 |
| Bridge 3 | 14 | B2 | 375 | Sulfentrazone | 420 | CHEAL | 21 | 100 |
| Bridge 3 | 15 | B2 | 560 | Sulfentrazone | 420 | CHEAL | 21 | 100 |
| Bridge 3 | 15 | B2 | 560 | Sulfentrazone | 420 | CHEAL | 21 | 100 |
| Bridge 3 | 15 | B2 | 560 | Sulfentrazone | 420 | CHEAL | 21 | 100 |
| Bridge 3 | 16 | B2 | 1120 | Sulfentrazone | 420 | CHEAL | 21 | 100 |
| Bridge 3 | 16 | B2 | 1120 | Sulfentrazone | 420 | CHEAL | 21 | 100 |
| Bridge 3 | 16 | B2 | 1120 | Sulfentrazone | 420 | CHEAL | 21 | 100 |
| Bridge 3 | 17 | D10 | 560 | none | 0 | CHEAL | 21 | 95 |
| Bridge 3 | 17 | D10 | 560 | none | 0 | CHEAL | 21 | 95 |
| Bridge 3 | 17 | D10 | 560 | none | 0 | CHEAL | 21 | 100 |
| Bridge 3 | 18 | D10 | 1120 | none | 0 | CHEAL | 21 | 100 |
| Bridge 3 | 18 | D10 | 1120 | none | 0 | CHEAL | 21 | 97 |
| Bridge 3 | 18 | D10 | 1120 | none | 0 | CHEAL | 21 | 100 |
| Bridge 3 | 19 | D10 | 560 | Sulfentrazone | 130 | CHEAL | 21 | 100 |
| Bridge 3 | 19 | D10 | 560 | Sulfentrazone | 130 | CHEAL | 21 | 100 |
| Bridge 3 | 19 | D10 | 560 | Sulfentrazone | 130 | CHEAL | 21 | 100 |
| Bridge 3 | 20 | D10 | 1120 | Sulfentrazone | 130 | CHEAL | 21 | 100 |
| Bridge 3 | 20 | D10 | 1120 | Sulfentrazone | 130 | CHEAL | 21 | 100 |
| Bridge 3 | 20 | D10 | 1120 | Sulfentrazone | 130 | CHEAL | 21 | 100 |
| Bridge 3 | 21 | D10 | 560 | Sulfentrazone | 347 | CHEAL | 21 | 100 |
| Bridge 3 | 21 | D10 | 560 | Sulfentrazone | 347 | CHEAL | 21 | 100 |
| Bridge 3 | 21 | D10 | 560 | Sulfentrazone | 347 | CHEAL | 21 | 100 |
| Bridge 3 | 22 | D10 | 1120 | Sulfentrazone | 347 | CHEAL | 21 | 100 |
| Bridge 3 | 22 | D10 | 1120 | Sulfentrazone | 347 | CHEAL | 21 | 100 |
| Bridge 3 | 22 | D10 | 1120 | Sulfentrazone | 347 | CHEAL | 21 | 100 |
| Bridge 3 | 23 | D8 | 560 | none | 0 | CHEAL | 21 | 100 |
| Bridge 3 | 23 | D8 | 560 | none | 0 | CHEAL | 21 | 95 |
| Bridge 3 | 23 | D8 | 560 | none | 0 | CHEAL | 21 | 95 |
| Bridge 3 | 24 | D8 | 1120 | none | 0 | CHEAL | 21 | 100 |
| Bridge 3 | 24 | D8 | 1120 | none | 0 | CHEAL | 21 | 100 |
| Bridge 3 | 24 | D8 | 1120 | none | 0 | CHEAL | 21 | 100 |
| Bridge 3 | 25 | D8 | 560 | Sulfentrazone | 130 | CHEAL | 21 | 100 |
| Bridge 3 | 25 | D8 | 560 | Sulfentrazone | 130 | CHEAL | 21 | 100 |
| Bridge 3 | 25 | D8 | 560 | Sulfentrazone | 130 | CHEAL | 21 | 100 |
| Bridge 3 | 26 | D8 | 1120 | Sulfentrazone | 130 | CHEAL | 21 | 100 |
| Bridge 3 | 26 | D8 | 1120 | Sulfentrazone | 130 | CHEAL | 21 | 100 |
| Bridge 3 | 26 | D8 | 1120 | Sulfentrazone | 130 | CHEAL | 21 | 100 |
| Bridge 3 | 27 | D8 | 560 | Sulfentrazone | 347 | CHEAL | 21 | 100 |
| Bridge 3 | 27 | D8 | 560 | Sulfentrazone | 347 | CHEAL | 21 | 100 |
| Bridge 3 | 27 | D8 | 560 | Sulfentrazone | 347 | CHEAL | 21 | 100 |
| Bridge 3 | 28 | D8 | 1120 | Sulfentrazone | 347 | CHEAL | 21 | 97 |
| Bridge 3 | 28 | D8 | 1120 | Sulfentrazone | 347 | CHEAL | 21 | 100 |
| Bridge 3 | 28 | D8 | 1120 | Sulfentrazone | 347 | CHEAL | 21 | 100 |
| Bridge 3 | 29 | D27 | 560 | none | 0 | CHEAL | 21 | 95 |
| Bridge 3 | 29 | D27 | 560 | none | 0 | CHEAL | 21 | 92 |
| Bridge 3 | 29 | D27 | 560 | none | 0 | CHEAL | 21 | 100 |
| Bridge 3 | 30 | D27 | 1120 | none | 0 | CHEAL | 21 | 100 |
| Bridge 3 | 30 | D27 | 1120 | none | 0 | CHEAL | 21 | 95 |
| Bridge 3 | 30 | D27 | 1120 | none | 0 | CHEAL | 21 | 97 |
| Bridge 3 | 31 | D27 | 560 | Sulfentrazone | 130 | CHEAL | 21 | 100 |
| Bridge 3 | 31 | D27 | 560 | Sulfentrazone | 130 | CHEAL | 21 | 100 |
| Bridge 3 | 31 | D27 | 560 | Sulfentrazone | 130 | CHEAL | 21 | 100 |
| Bridge 3 | 32 | D27 | 1120 | Sulfentrazone | 130 | CHEAL | 21 | 100 |
| Bridge 3 | 32 | D27 | 1120 | Sulfentrazone | 130 | CHEAL | 21 | 100 |
| Bridge 3 | 32 | D27 | 1120 | Sulfentrazone | 130 | CHEAL | 21 | 100 |
| Bridge 3 | 33 | D27 | 560 | Sulfentrazone | 347 | CHEAL | 21 | 100 |
| Bridge 3 | 33 | D27 | 560 | Sulfentrazone | 347 | CHEAL | 21 | 100 |
| Bridge 3 | 33 | D27 | 560 | Sulfentrazone | 347 | CHEAL | 21 | 100 |
| Bridge 3 | 34 | D27 | 1120 | Sulfentrazone | 347 | CHEAL | 21 | 100 |
| Bridge 3 | 34 | D27 | 1120 | Sulfentrazone | 347 | CHEAL | 21 | 100 |
| Bridge 3 | 34 | D27 | 1120 | Sulfentrazone | 347 | CHEAL | 21 | 100 |
| Bridge 3 | 35 | Untreated | | | | CHEAL | 21 | 0 |
| Bridge 3 | 35 | Untreated | | | | CHEAL | 21 | 0 |
| Bridge 3 | 35 | Untreated | | | | CHEAL | 21 | 0 |
| Bridge 3 | 1 | Solv. Ck | 0 | 0 | 0 | ERICA | 21 | 0 |
| | | untreated | | | | | | |
| Bridge 3 | 1 | Solv. Ck | 0 | 0 | 0 | ERICA | 21 | 0 |
| | | untreated | | | | | | |
| Bridge 3 | 1 | Solv. Ck | 0 | 0 | 0 | ERICA | 21 | 0 |
| | | untreated | | | | | | |
| Bridge 3 | 2 | B2 | 375 | none | | ERICA | 21 | 80 |
| Bridge 3 | 2 | B2 | 375 | none | | ERICA | 21 | 75 |
| Bridge 3 | 2 | B2 | 375 | none | | ERICA | 21 | 97 |
| Bridge 3 | 3 | B2 | 560 | none | | ERICA | 21 | 75 |
| Bridge 3 | 3 | B2 | 560 | none | | ERICA | 21 | 85 |
| Bridge 3 | 3 | B2 | 560 | none | | ERICA | 21 | 97 |
| Bridge 3 | 4 | B2 | 1120 | none | | ERICA | 21 | 90 |
| Bridge 3 | 4 | B2 | 1120 | none | | ERICA | 21 | 90 |
| Bridge 3 | 4 | B2 | 1120 | none | | ERICA | 21 | 98 |
| Bridge 3 | 5 | B2 | 0 | Sulfentrazone | 140 | ERICA | 21 | 20 |
| Bridge 3 | 5 | B2 | 0 | Sulfentrazone | 140 | ERICA | 21 | 15 |
| Bridge 3 | 5 | B2 | 0 | Sulfentrazone | 140 | ERICA | 21 | 10 |
| Bridge 3 | 6 | B2 | 375 | Sulfentrazone | 140 | ERICA | 21 | 98 |
| Bridge 3 | 6 | B2 | 375 | Sulfentrazone | 140 | ERICA | 21 | 80 |
| Bridge 3 | 6 | B2 | 375 | Sulfentrazone | 140 | ERICA | 21 | 97 |
| Bridge 3 | 7 | B2 | 560 | Sulfentrazone | 140 | ERICA | 21 | 90 |
| Bridge 3 | 7 | B2 | 560 | Sulfentrazone | 140 | ERICA | 21 | 85 |
| Bridge 3 | 7 | B2 | 560 | Sulfentrazone | 140 | ERICA | 21 | 95 |
| Bridge 3 | 8 | B2 | 1120 | Sulfentrazone | 140 | ERICA | 21 | 85 |
| Bridge 3 | 8 | B2 | 1120 | Sulfentrazone | 140 | ERICA | 21 | 97 |
| Bridge 3 | 8 | B2 | 1120 | Sulfentrazone | 140 | ERICA | 21 | 97 |
| Bridge 3 | 9 | B2 | 0 | Sulfentrazone | 210 | ERICA | 21 | 15 |
| Bridge 3 | 9 | B2 | 0 | Sulfentrazone | 210 | ERICA | 21 | 20 |
| Bridge 3 | 9 | B2 | 0 | Sulfentrazone | 210 | ERICA | 21 | 20 |
| Bridge 3 | 10 | B2 | 375 | Sulfentrazone | 210 | ERICA | 21 | 90 |
| Bridge 3 | 10 | B2 | 375 | Sulfentrazone | 210 | ERICA | 21 | 80 |
| Bridge 3 | 10 | B2 | 375 | Sulfentrazone | 210 | ERICA | 21 | 75 |
| Bridge 3 | 11 | B2 | 560 | Sulfentrazone | 210 | ERICA | 21 | 95 |
| Bridge 3 | 11 | B2 | 560 | Sulfentrazone | 210 | ERICA | 21 | 85 |
| Bridge 3 | 11 | B2 | 560 | Sulfentrazone | 210 | ERICA | 21 | 95 |
| Bridge 3 | 12 | B2 | 1120 | Sulfentrazone | 210 | ERICA | 21 | 97 |
| Bridge 3 | 12 | B2 | 1120 | Sulfentrazone | 210 | ERICA | 21 | 95 |
| Bridge 3 | 12 | B2 | 1120 | Sulfentrazone | 210 | ERICA | 21 | 99 |
| Bridge 3 | 13 | B2 | 0 | Sulfentrazone | 420 | ERICA | 21 | 30 |
| Bridge 3 | 13 | B2 | 0 | Sulfentrazone | 420 | ERICA | 21 | 20 |
| Bridge 3 | 13 | B2 | 0 | Sulfentrazone | 420 | ERICA | 21 | 20 |
| Bridge 3 | 14 | B2 | 375 | Sulfentrazone | 420 | ERICA | 21 | 75 |
| Bridge 3 | 14 | B2 | 375 | Sulfentrazone | 420 | ERICA | 21 | 75 |
| Bridge 3 | 14 | B2 | 375 | Sulfentrazone | 420 | ERICA | 21 | 85 |
| Bridge 3 | 15 | B2 | 560 | Sulfentrazone | 420 | ERICA | 21 | 70 |
| Bridge 3 | 15 | B2 | 560 | Sulfentrazone | 420 | ERICA | 21 | 90 |
| Bridge 3 | 15 | B2 | 560 | Sulfentrazone | 420 | ERICA | 21 | 95 |
| Bridge 3 | 16 | B2 | 1120 | Sulfentrazone | 420 | ERICA | 21 | 95 |
| Bridge 3 | 16 | B2 | 1120 | Sulfentrazone | 420 | ERICA | 21 | 90 |
| Bridge 3 | 16 | B2 | 1120 | Sulfentrazone | 420 | ERICA | 21 | 95 |
| Bridge 3 | 17 | D10 | 560 | none | 0 | ERICA | 21 | 80 |
| Bridge 3 | 17 | D10 | 560 | none | 0 | ERICA | 21 | 80 |
| Bridge 3 | 17 | D10 | 560 | none | 0 | ERICA | 21 | 95 |
| Bridge 3 | 18 | D10 | 1120 | none | 0 | ERICA | 21 | 75 |
| Bridge 3 | 18 | D10 | 1120 | none | 0 | ERICA | 21 | 90 |
| Bridge 3 | 18 | D10 | 1120 | none | 0 | ERICA | 21 | 95 |
| Bridge 3 | 19 | D10 | 560 | Sulfentrazone | 130 | ERICA | 21 | 85 |
| Bridge 3 | 19 | D10 | 560 | Sulfentrazone | 130 | ERICA | 21 | 80 |
| Bridge 3 | 19 | D10 | 560 | Sulfentrazone | 130 | ERICA | 21 | 90 |
| Bridge 3 | 20 | D10 | 1120 | Sulfentrazone | 130 | ERICA | 21 | 90 |
| Bridge 3 | 20 | D10 | 1120 | Sulfentrazone | 130 | ERICA | 21 | 90 |
| Bridge 3 | 20 | D10 | 1120 | Sulfentrazone | 130 | ERICA | 21 | 95 |
| Bridge 3 | 21 | D10 | 560 | Sulfentrazone | 347 | ERICA | 21 | 90 |
| Bridge 3 | 21 | D10 | 560 | Sulfentrazone | 347 | ERICA | 21 | 97 |
| Bridge 3 | 21 | D10 | 560 | Sulfentrazone | 347 | ERICA | 21 | 98 |
| Bridge 3 | 22 | D10 | 1120 | Sulfentrazone | 347 | ERICA | 21 | 95 |
| Bridge 3 | 22 | D10 | 1120 | Sulfentrazone | 347 | ERICA | 21 | 97 |
| Bridge 3 | 22 | D10 | 1120 | Sulfentrazone | 347 | ERICA | 21 | 98 |
| Bridge 3 | 23 | D8 | 560 | none | 0 | ERICA | 21 | 95 |
| Bridge 3 | 23 | D8 | 560 | none | 0 | ERICA | 21 | 90 |
| Bridge 3 | 23 | D8 | 560 | none | 0 | ERICA | 21 | 80 |
| Bridge 3 | 24 | D8 | 1120 | none | 0 | ERICA | 21 | 95 |
| Bridge 3 | 24 | D8 | 1120 | none | 0 | ERICA | 21 | 90 |
| Bridge 3 | 24 | D8 | 1120 | none | 0 | ERICA | 21 | 95 |
| Bridge 3 | 25 | D8 | 560 | Sulfentrazone | 130 | ERICA | 21 | 97 |
| Bridge 3 | 25 | D8 | 560 | Sulfentrazone | 130 | ERICA | 21 | 85 |
| Bridge 3 | 25 | D8 | 560 | Sulfentrazone | 130 | ERICA | 21 | 95 |
| Bridge 3 | 26 | D8 | 1120 | Sulfentrazone | 130 | ERICA | 21 | 95 |
| Bridge 3 | 26 | D8 | 1120 | Sulfentrazone | 130 | ERICA | 21 | 95 |
| Bridge 3 | 26 | D8 | 1120 | Sulfentrazone | 130 | ERICA | 21 | 99 |
| Bridge 3 | 27 | D8 | 560 | Sulfentrazone | 347 | ERICA | 21 | 100 |
| Bridge 3 | 27 | D8 | 560 | Sulfentrazone | 347 | ERICA | 21 | 98 |
| Bridge 3 | 27 | D8 | 560 | Sulfentrazone | 347 | ERICA | 21 | 97 |
| Bridge 3 | 28 | D8 | 1120 | Sulfentrazone | 347 | ERICA | 21 | 95 |
| Bridge 3 | 28 | D8 | 1120 | Sulfentrazone | 347 | ERICA | 21 | 85 |
| Bridge 3 | 28 | D8 | 1120 | Sulfentrazone | 347 | ERICA | 21 | 98 |
| Bridge 3 | 29 | D27 | 560 | none | 0 | ERICA | 21 | 85 |
| Bridge 3 | 29 | D27 | 560 | none | 0 | ERICA | 21 | 85 |
| Bridge 3 | 29 | D27 | 560 | none | 0 | ERICA | 21 | 80 |
| Bridge 3 | 30 | D27 | 1120 | none | 0 | ERICA | 21 | 95 |
| Bridge 3 | 30 | D27 | 1120 | none | 0 | ERICA | 21 | 85 |
| Bridge 3 | 30 | D27 | 1120 | none | 0 | ERICA | 21 | 95 |
| Bridge 3 | 31 | D27 | 560 | Sulfentrazone | 130 | ERICA | 21 | 80 |
| Bridge 3 | 31 | D27 | 560 | Sulfentrazone | 130 | ERICA | 21 | 95 |
| Bridge 3 | 31 | D27 | 560 | Sulfentrazone | 130 | ERICA | 21 | 75 |
| Bridge 3 | 32 | D27 | 1120 | Sulfentrazone | 130 | ERICA | 21 | 95 |
| Bridge 3 | 32 | D27 | 1120 | Sulfentrazone | 130 | ERICA | 21 | 90 |
| Bridge 3 | 32 | D27 | 1120 | Sulfentrazone | 130 | ERICA | 21 | 95 |
| Bridge 3 | 33 | D27 | 560 | Sulfentrazone | 347 | ERICA | 21 | 75 |
| Bridge 3 | 33 | D27 | 560 | Sulfentrazone | 347 | ERICA | 21 | 85 |
| Bridge 3 | 33 | D27 | 560 | Sulfentrazone | 347 | ERICA | 21 | 90 |
| Bridge 3 | 34 | D27 | 1120 | Sulfentrazone | 347 | ERICA | 21 | 90 |
| Bridge 3 | 34 | D27 | 1120 | Sulfentrazone | 347 | ERICA | 21 | 90 |
| Bridge 3 | 34 | D27 | 1120 | Sulfentrazone | 347 | ERICA | 21 | 85 |
| Bridge 3 | 35 | Untreated | | | | ERICA | 21 | 0 |
| Bridge 3 | 35 | Untreated | | | | ERICA | 21 | 0 |
| Bridge 3 | 35 | Untreated | | | | ERICA | 21 | 0 |
| Bridge 3 | 1 | Solv. Ck | 0 | 0 | 0 | ZEAMX | 21 | 0 |
| | | untreated | | | | | | |
| Bridge 3 | 1 | Solv. Ck | 0 | 0 | 0 | ZEAMX | 21 | 0 |
| | | untreated | | | | | | |
| Bridge 3 | 1 | Solv. Ck | 0 | 0 | 0 | ZEAMX | 21 | 0 |
| | | untreated | | | | | | |
| Bridge 3 | 2 | B2 | 375 | none | | ZEAMX | 21 | 5 |
| Bridge 3 | 2 | B2 | 375 | none | | ZEAMX | 21 | 5 |
| Bridge 3 | 2 | B2 | 375 | none | | ZEAMX | 21 | 5 |
| Bridge 3 | 3 | B2 | 560 | none | | ZEAMX | 21 | 5 |
| Bridge 3 | 3 | B2 | 560 | none | | ZEAMX | 21 | 10 |
| Bridge 3 | 3 | B2 | 560 | none | | ZEAMX | 21 | 5 |
| Bridge 3 | 4 | B2 | 1120 | none | | ZEAMX | 21 | 5 |
| Bridge 3 | 4 | B2 | 1120 | none | | ZEAMX | 21 | 5 |
| Bridge 3 | 4 | B2 | 1120 | none | | ZEAMX | 21 | 5 |
| Bridge 3 | 5 | B2 | 0 | Sulfentrazone | 140 | ZEAMX | 21 | 40 |
| Bridge 3 | 5 | B2 | 0 | Sulfentrazone | 140 | ZEAMX | 21 | 40 |
| Bridge 3 | 5 | B2 | 0 | Sulfentrazone | 140 | ZEAMX | 21 | 50 |
| Bridge 3 | 6 | B2 | 375 | Sulfentrazone | 140 | ZEAMX | 21 | 50 |
| Bridge 3 | 6 | B2 | 375 | Sulfentrazone | 140 | ZEAMX | 21 | 85 |
| Bridge 3 | 6 | B2 | 375 | Sulfentrazone | 140 | ZEAMX | 21 | 60 |
| Bridge 3 | 7 | B2 | 560 | Sulfentrazone | 140 | ZEAMX | 21 | 70 |
| Bridge 3 | 7 | B2 | 560 | Sulfentrazone | 140 | ZEAMX | 21 | 60 |
| Bridge 3 | 7 | B2 | 560 | Sulfentrazone | 140 | ZEAMX | 21 | 65 |
| Bridge 3 | 8 | B2 | 1120 | Sulfentrazone | 140 | ZEAMX | 21 | 45 |
| Bridge 3 | 8 | B2 | 1120 | Sulfentrazone | 140 | ZEAMX | 21 | 65 |
| Bridge 3 | 8 | B2 | 1120 | Sulfentrazone | 140 | ZEAMX | 21 | 50 |
| Bridge 3 | 9 | B2 | 0 | Sulfentrazone | 210 | ZEAMX | 21 | 40 |
| Bridge 3 | 9 | B2 | 0 | Sulfentrazone | 210 | ZEAMX | 21 | 60 |
| Bridge 3 | 9 | B2 | 0 | Sulfentrazone | 210 | ZEAMX | 21 | 50 |
| Bridge 3 | 10 | B2 | 375 | Sulfentrazone | 210 | ZEAMX | 21 | 70 |
| Bridge 3 | 10 | B2 | 375 | Sulfentrazone | 210 | ZEAMX | 21 | 80 |
| Bridge 3 | 10 | B2 | 375 | Sulfentrazone | 210 | ZEAMX | 21 | 75 |
| Bridge 3 | 11 | B2 | 560 | Sulfentrazone | 210 | ZEAMX | 21 | 50 |
| Bridge 3 | 11 | B2 | 560 | Sulfentrazone | 210 | ZEAMX | 21 | 55 |
| Bridge 3 | 11 | B2 | 560 | Sulfentrazone | 210 | ZEAMX | 21 | 70 |
| Bridge 3 | 12 | B2 | 1120 | Sulfentrazone | 210 | ZEAMX | 21 | 90 |
| Bridge 3 | 12 | B2 | 1120 | Sulfentrazone | 210 | ZEAMX | 21 | 50 |
| Bridge 3 | 12 | B2 | 1120 | Sulfentrazone | 210 | ZEAMX | 21 | 75 |
| Bridge 3 | 13 | B2 | 0 | Sulfentrazone | 420 | ZEAMX | 21 | 75 |
| Bridge 3 | 13 | B2 | 0 | Sulfentrazone | 420 | ZEAMX | 21 | 40 |
| Bridge 3 | 13 | B2 | 0 | Sulfentrazone | 420 | ZEAMX | 21 | 60 |
| Bridge 3 | 14 | B2 | 375 | Sulfentrazone | 420 | ZEAMX | 21 | 60 |
| Bridge 3 | 14 | B2 | 375 | Sulfentrazone | 420 | ZEAMX | 21 | 65 |
| Bridge 3 | 14 | B2 | 375 | Sulfentrazone | 420 | ZEAMX | 21 | 90 |
| Bridge 3 | 15 | B2 | 560 | Sulfentrazone | 420 | ZEAMX | 21 | 60 |
| Bridge 3 | 15 | B2 | 560 | Sulfentrazone | 420 | ZEAMX | 21 | 70 |
| Bridge 3 | 15 | B2 | 560 | Sulfentrazone | 420 | ZEAMX | 21 | 65 |
| Bridge 3 | 16 | B2 | 1120 | Sulfentrazone | 420 | ZEAMX | 21 | 85 |
| Bridge 3 | 16 | B2 | 1120 | Sulfentrazone | 420 | ZEAMX | 21 | 75 |
| Bridge 3 | 16 | B2 | 1120 | Sulfentrazone | 420 | ZEAMX | 21 | 50 |
| Bridge 3 | 17 | D10 | 560 | none | 0 | ZEAMX | 21 | 5 |
| Bridge 3 | 17 | D10 | 560 | none | 0 | ZEAMX | 21 | 10 |
| Bridge 3 | 17 | D10 | 560 | none | 0 | ZEAMX | 21 | 5 |
| Bridge 3 | 18 | D10 | 1120 | none | 0 | ZEAMX | 21 | 5 |
| Bridge 3 | 18 | D10 | 1120 | none | 0 | ZEAMX | 21 | 15 |
| Bridge 3 | 18 | D10 | 1120 | none | 0 | ZEAMX | 21 | 5 |
| Bridge 3 | 19 | D10 | 560 | Sulfentrazone | 130 | ZEAMX | 21 | 30 |
| Bridge 3 | 19 | D10 | 560 | Sulfentrazone | 130 | ZEAMX | 21 | 20 |
| Bridge 3 | 19 | D10 | 560 | Sulfentrazone | 130 | ZEAMX | 21 | 60 |
| Bridge 3 | 20 | D10 | 1120 | Sulfentrazone | 130 | ZEAMX | 21 | 50 |
| Bridge 3 | 20 | D10 | 1120 | Sulfentrazone | 130 | ZEAMX | 21 | 60 |
| Bridge 3 | 20 | D10 | 1120 | Sulfentrazone | 130 | ZEAMX | 21 | 70 |
| Bridge 3 | 21 | D10 | 560 | Sulfentrazone | 347 | ZEAMX | 21 | 40 |
| Bridge 3 | 21 | D10 | 560 | Sulfentrazone | 347 | ZEAMX | 21 | 50 |
| Bridge 3 | 21 | D10 | 560 | Sulfentrazone | 347 | ZEAMX | 21 | 50 |
| Bridge 3 | 22 | D10 | 1120 | Sulfentrazone | 347 | ZEAMX | 21 | 65 |
| Bridge 3 | 22 | D10 | 1120 | Sulfentrazone | 347 | ZEAMX | 21 | 55 |
| Bridge 3 | 22 | D10 | 1120 | Sulfentrazone | 347 | ZEAMX | 21 | 65 |
| Bridge 3 | 23 | D8 | 560 | none | 0 | ZEAMX | 21 | 10 |
| Bridge 3 | 23 | D8 | 560 | none | 0 | ZEAMX | 21 | 5 |
| Bridge 3 | 23 | D8 | 560 | none | 0 | ZEAMX | 21 | 10 |
| Bridge 3 | 24 | D8 | 1120 | none | 0 | ZEAMX | 21 | 5 |
| Bridge 3 | 24 | D8 | 1120 | none | 0 | ZEAMX | 21 | 5 |
| Bridge 3 | 24 | D8 | 1120 | none | 0 | ZEAMX | 21 | 5 |
| Bridge 3 | 25 | D8 | 560 | Sulfentrazone | 130 | ZEAMX | 21 | 75 |
| Bridge 3 | 25 | D8 | 560 | Sulfentrazone | 130 | ZEAMX | 21 | 65 |
| Bridge 3 | 25 | D8 | 560 | Sulfentrazone | 130 | ZEAMX | 21 | 65 |
| Bridge 3 | 26 | D8 | 1120 | Sulfentrazone | 130 | ZEAMX | 21 | 50 |
| Bridge 3 | 26 | D8 | 1120 | Sulfentrazone | 130 | ZEAMX | 21 | 75 |
| Bridge 3 | 26 | D8 | 1120 | Sulfentrazone | 130 | ZEAMX | 21 | 85 |
| Bridge 3 | 27 | D8 | 560 | Sulfentrazone | 347 | ZEAMX | 21 | 55 |
| Bridge 3 | 27 | D8 | 560 | Sulfentrazone | 347 | ZEAMX | 21 | 75 |
| Bridge 3 | 27 | D8 | 560 | Sulfentrazone | 347 | ZEAMX | 21 | 60 |
| Bridge 3 | 28 | D8 | 1120 | Sulfentrazone | 347 | ZEAMX | 21 | 85 |
| Bridge 3 | 28 | D8 | 1120 | Sulfentrazone | 347 | ZEAMX | 21 | 65 |
| Bridge 3 | 28 | D8 | 1120 | Sulfentrazone | 347 | ZEAMX | 21 | 75 |
| Bridge 3 | 29 | D2 | 560 | none | 0 | ZEAMX | 21 | 5 |
| Bridge 3 | 29 | D27 | 560 | none | 0 | ZEAMX | 21 | 5 |
| Bridge 3 | 29 | D27 | 560 | none | 0 | ZEAMX | 21 | 5 |
| Bridge 3 | 30 | D27 | 1120 | none | 0 | ZEAMX | 21 | 5 |
| Bridge 3 | 30 | D27 | 1120 | none | 0 | ZEAMX | 21 | 5 |
| Bridge 3 | 30 | D27 | 1120 | none | 0 | ZEAMX | 21 | 10 |
| Bridge 3 | 31 | D27 | 560 | Sulfentrazone | 130 | ZEAMX | 21 | 85 |
| Bridge 3 | 31 | D27 | 560 | Sulfentrazone | 130 | ZEAMX | 21 | 85 |
| Bridge 3 | 31 | D27 | 560 | Sulfentrazone | 130 | ZEAMX | 21 | 60 |
| Bridge 3 | 32 | D27 | 1120 | Sulfentrazone | 130 | ZEAMX | 21 | 80 |
| Bridge 3 | 32 | D27 | 1120 | Sulfentrazone | 130 | ZEAMX | 21 | 80 |
| Bridge 3 | 32 | D27 | 1120 | Sulfentrazone | 130 | ZEAMX | 21 | 75 |
| Bridge 3 | 33 | D27 | 560 | Sulfentrazone | 347 | ZEAMX | 21 | 60 |
| Bridge 3 | 33 | D27 | 560 | Sulfentrazone | 347 | ZEAMX | 21 | 40 |
| Bridge 3 | 33 | D27 | 560 | Sulfentrazone | 347 | ZEAMX | 21 | 75 |
| Bridge 3 | 34 | D27 | 1120 | Sulfentrazone | 347 | ZEAMX | 21 | 90 |
| Bridge 3 | 34 | D27 | 1120 | Sulfentrazone | 347 | ZEAMX | 21 | 70 |
| Bridge 3 | 34 | D27 | 1120 | Sulfentrazone | 347 | ZEAMX | 21 | 75 |
| Bridge 3 | 35 | Untreated | | | | ZEAMX | 21 | 0 |
| Bridge 3 | 35 | Untreated | | | | ZEAMX | 21 | 0 |

While this disclosure has been described using exemplary details, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

The benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.”

In the detailed description herein, references to “one aspect,” “an aspect,” “an example aspect,” “detail” etc., indicate that the aspect described may include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it is submitted that it is within the knowledge of one skilled in the art with the benefit of the present disclosure to affect such feature, structure, or characteristic in connection with other aspects whether or not explicitly described. After reading the description, it will be apparent to one skilled in the relevant art(s) how to implement the disclosure in alternative aspects.

Furthermore, no element, component, or method step in the present disclosure is intended to be dedicated to the public regardless of whether the element, component, or method step is explicitly recited in the claims.

1. A compound according to Formula I: 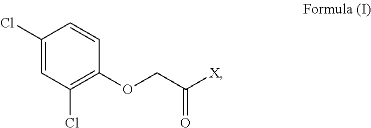
wherein X is: 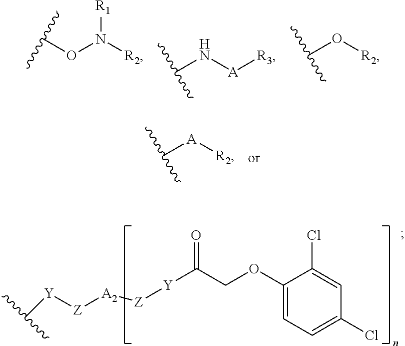
wherein
R₁ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, or heteroarylthiocarbonyl;
R₂ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl;
R₃ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl;
R₁ and R₂ together form a substituted double bond, a 3-membered ring, a 4-membered, a 5-membered ring, a 6-membered or a 7-membered ring;
A is independently O, Si, NR, and S(O)ₘ;
A₂ is independently O, N, S(O)ₘ, substituted alkyl, heteroalkyl, alkenyl, alkynyl, aryl, heteroaryl, alkylaryl, alkylheteroaryl;
Y is independently O, N, S;
Z is independently O, N, S(O)ₘ, substituted alkyl, alkenyl, alkynyl, aryl, heteroaryl, alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl, —N═R₄R₅,
R₄ and R₅ are independently substituted amino, alkoxy, alkyl, heteroalkyl, aryl, heteroaryl;
n is 1, 2, or 3; and
m is 0, 1, or 2; wherein
the compound exhibits pesticidal activity with the proviso that the compound is not 2,4-D isooctyl ester.

2. The compound according to claim 1, wherein X is: 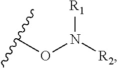
where
R₁ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, or heteroarylthiocarbonyl; and
R₂ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl.

3. The compound according to claim 1, wherein X is: 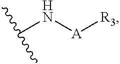
where
R₃ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl;
A is independently O, Si, NR, and S(O)ₘ, substituted alkyl, alkenyl, alkynyl, aryl, heteroaryl, alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl, —N═R₄R₅,
R₄ and R₅ are independently substituted amino, alkoxy, alkyl, heteroalkyl, aryl, heteroaryl; and
m is 0, 1, or 2.

4. The compound according to claim 1, wherein X is: 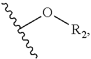
where
R₂ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl.

5. The compound according to claim 1, wherein X is:

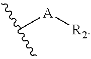

6. The compound according to claim 1, wherein X is: 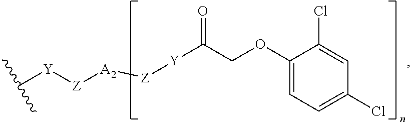
where
A₂ is independently O, N, S(O)ₘ, substituted alkyl, heteroalkyl, alkenyl, alkynyl, aryl, heteroaryl, alkylaryl, alkylheteroaryl;
Y is independently O, N, S;
Z is independently O, N, S(O)ₘ, substituted alkyl, alkenyl, alkynyl, aryl, heteroaryl, alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl, —N═R₄R₅,
R₄ and R₅ are independently substituted amino, alkoxy, alkyl, heteroalkyl, aryl, heteroaryl;
n is 1, 2, or 3; and
m is 0, 1, or 2.

7. The compound according to claim 2, wherein the compound is according to the following Formula A1: 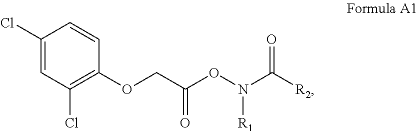
wherein
R₁ and R₂ are independently hydrogen, C₁-C₁₆ alkyl, C₁-C₁₆ heteroalkyl, C₅-C₁₀ aryl, C₅-C₁₀ heteroaryl, alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl, mono- or disubstituted alkylamino, mono- or disubstituted arylamino, mono- or disubstituted heteroarylamino, alkoxy, arylalkoxy, heteroarylalkoxy, alkylthio, arylthio, heterarythio.

8. The compound according to claim 2, wherein the compound is according to the following Formula A2: 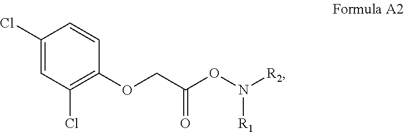
wherein
R₁ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, or heteroarylthiocarbonyl; and
R₂ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl.

9. The compound according to claim 2, wherein the compound is according to the following Formula A3: 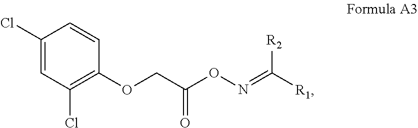
wherein
R₁ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, or heteroarylthiocarbonyl; and
R₂ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl.

10. The compound according to claim 2, wherein the compound is according to the following Formula A4: 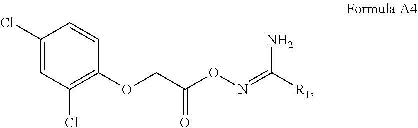
wherein
R₁ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, or heteroarylthiocarbonyl.

11. The compound according to claim 2, wherein the compound is selected from the group consisting of A1, A2, A3, A4, A5, A6, A7, A8, A9, A10, A11, A12, A13, A14, A15, A16, A17, A18, A19, A20, A21, A22, A23, A24, A25, A26, A27, A28, A29, A30, A31, A32, A33, A34, A35, A36, A37, A38, A39, A40, and A41.

12. The compound according to claim 6, wherein the compound is according to the following Formula B1:

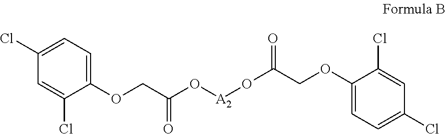

13. The compound according to claim 6, selected from the group consisting of compounds B1, B2, B3, B4, B5, B6, B7, B8, B9, B10, B11, B12, B13, B14, and B15.

14. The compound according to claim 13, wherein the compound is selected from the group consisting of:

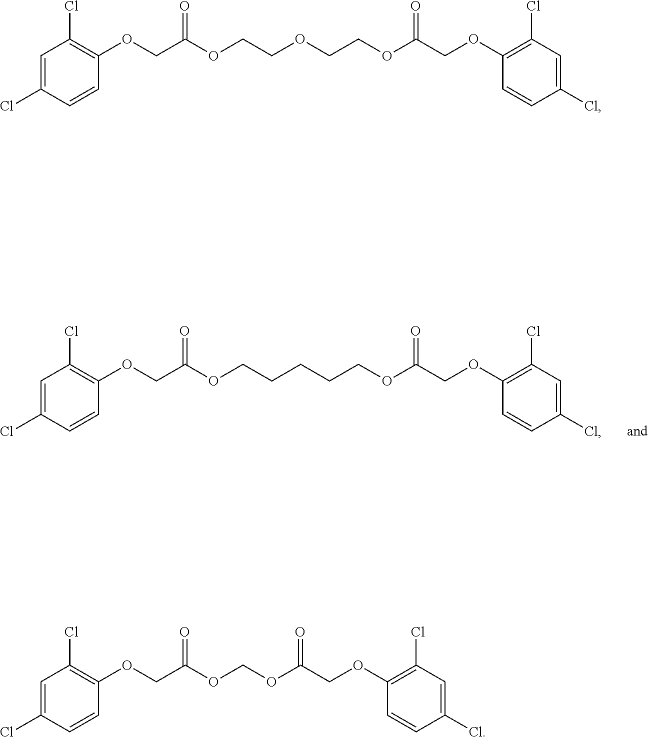 [SMILES: O=C(COc1ccc(Cl)cc1Cl)OCCOCCOC(=O)COc1ccc(Cl)cc1Cl ; O=C(COc1ccc(Cl)cc1Cl)OCCCCCOC(=O)COc1ccc(Cl)cc1Cl ; O=C(COc1ccc(Cl)cc1Cl)OCOC(=O)COc1ccc(Cl)cc1Cl]

15. The compound according to claim 5, wherein the compound is according to the following Formula C: 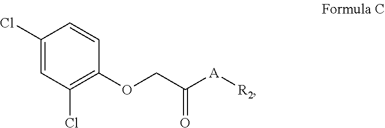
where
A is independently C;
R₂ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl.

16. The compound according to claim 2, wherein the compound is according to the following Formula D: 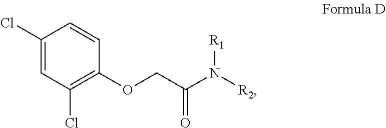
where
R₁ and R₂ are independently hydrogen, C₁-C₁₆ alkyl, C₁-C₁₆ heteroalkyl, C₅-C₁₀ aryl, C₅-C₁₀ heteroaryl, alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl, alkylsulfonyl, arylsulfonyl, heteroarylsulfonyl, alkoxysulfonyl, alkylaminosulfonyl, arylaminosulfonyl, heteroarylaminosulfonyl.

17. The compound according to claim 3, wherein the compound is: 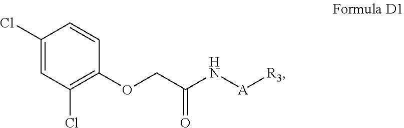
where
R₃ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl; and
A is independently O, Si, or NR₁, S(O)n where n=0, 1, 2, and R₃ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl.

18. The compound according to claim 2, wherein the compound is: 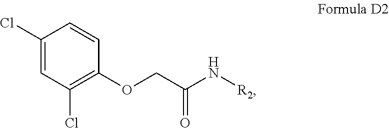
where R₂ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl, alkylsulfonyl, arylsulfonyl, heteroarylsulfonyl, alkoxysulfonyl, alkylaminosulfonyl, arylaminosulfonyl, heteroarylaminosulfonyl.

19. The compound according to claim 16, wherein the compound is:

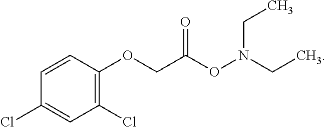 [SMILES: CCN(CC)OC(=O)COc1ccc(Cl)cc1Cl]

20. The compound according to claim 1, wherein the compound is selected from the group consisting of D1, D2, D3, D4, D5, D6, D7, D8, D9, D10, D11, D12, D13, D14, D15, D16, D17, D18, D19, D20, D21, D22, D23, D24, D25, D26, D27, D28, D29, D30, D31, D32, and D33.

21. The compound according to claim 4, wherein the compound is according to the following Formula E: 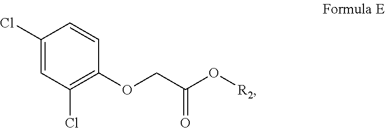
where
R₂ is independently hydrogen, C₁-C₁₆ alkyl, C₁-C₁₆ heteroalkyl, C₅-C₁₀ aryl, C₅-C₁₀ heteroaryl; alkylcarbonyl, alkoxycabonyl, aryloxycabonyl, heteroaryloxycabonyl, mono- or disubstituted alkylaminocarbonyl, mono- or disubstituted arylaminocarbonyl, mono- or disubstituted heteroarylaminocarbonyl, alkylthiocarbonyl, arylthiocarbonyl, heteroarylthiocarbonyl.

22. The compound according to claim 18, wherein the compound is selected from the group consisting of:

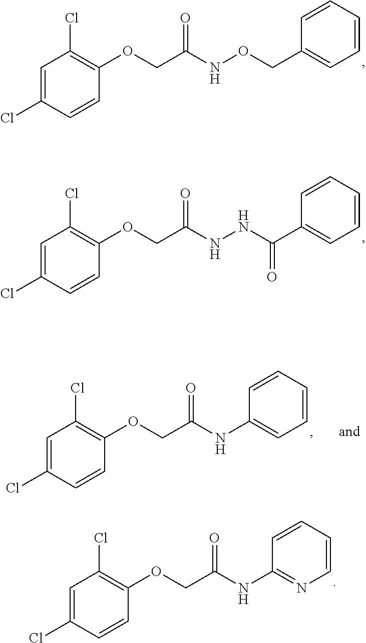 [SMILES: O=C(COc1ccc(Cl)cc1Cl)NOCc1ccccc1 ; O=C(COc1ccc(Cl)cc1Cl)NNC(=O)c1ccccc1 ; O=C(COc1ccc(Cl)cc1Cl)Nc1ccccc1 ; O=C(COc1ccc(Cl)cc1Cl)Nc1ccccn1]

23. The compound according to claim 1, wherein the compound is a compound listed in Table 1: Pesticidal Compounds.

24-42. (canceled)
